(19) United States
(12) Patent Application Publication
(10) Pub. No.: US 2023/0057899 A1
(43) Pub. Date: Feb. 23, 2023

(54) ANTI-PVRIG AND ANTI-TIGIT ANTIBODIES FOR ENHANCED NK-CELL BASED TUMOR KILLING

(71) Applicants: Compugen Ltd., Holon (IL); Peter MacCallum Cancer Centre, Melbourne (AU)

(72) Inventors: Maya Kotturi, Holon (IL); Sarah Whelan, Holon (IL); Paul Neeson, East Ivanhoe (AU); Jessica Li, Southbank (AU); Joseph A. Trapani, Camberwell (AU); Eran Ophir, Even Yehuda (IL); Moran Galperin, Ashdod (IL)

(21) Appl. No.: 17/782,635

(22) PCT Filed: Dec. 7, 2020
(86) PCT No.: PCT/US20/63643

§ 371 Date: Jun. 3, 2022

Related U.S. Application Data
(60) Provisional application No. 63/106,739, filed on Oct. 28, 2020.
(60) Provisional application No. 63/079,368, filed on Sep. 16, 2020.
(60) Provisional application No. 63/023,176, filed on May 11, 2020.
(60) Provisional application No. 62/944,096, filed on Dec. 5, 2019.

(51) Int. Cl. C07K 16/28; A61P 7/00; A61P 35/02

(52) U.S. Cl. CPC C07K 16/2803; C07K 16/28; A61P 7/00; A61P 35/02; A61K 2039/505

(57) ABSTRACT

Anti-PVRIG and anti-TIGIT for use in methods of treatment based on the enhanced NK-cell using the antibodies for the treatment of cancer.

### CROSS REFERENCE TO RELATED APPLICATIONS

This application claims priority to U.S. Patent Application No. 63/106,739 filed Oct. 28, 2020, U.S. Patent Application No. 63/079,368 filed Sep. 16, 2020, U.S. Patent Application No. 63/023,176 filed May 11, 2020, and U.S. Patent Application No. 62/944,096 filed Dec. 5, 2019, which are hereby incorporated by reference in their entireties.

### I. BACKGROUND OF THE INVENTION

Naïve T cells must receive two independent signals from antigen-presenting cells (APC) in order to become productively activated. The first, Signal 1, is antigen-specific and occurs when T cell antigen receptors encounter the appropriate antigen-MHC complex on the APC. The fate of the immune response is determined by a second, antigen-independent signal (Signal 2) which is delivered through a T cell costimulatory molecule that engages its APC-expressed ligand. This second signal could be either stimulatory (positive costimulation) or inhibitory (negative costimulation or coinhibition). In the absence of a costimulatory signal, or in the presence of a coinhibitory signal, T-cell activation is impaired or aborted, which may lead to a state of antigen-specific unresponsiveness (known as T-cell anergy), or may result in T-cell apoptotic death.

Costimulatory molecule pairs usually consist of ligands expressed on APCs and their cognate receptors expressed on T cells. The prototype ligand/receptor pairs of costimulatory molecules are B7/CD28 and CD40/CD40L. The B7 family consists of structurally related, cell-surface protein ligands, which may provide stimulatory or inhibitory input to an immune response. Members of the B7 family are structurally related, with the extracellular domain containing at least one variable or constant immunoglobulin domain.

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

Manipulation of the signals delivered by B7 ligands has shown potential in the treatment of autoimmunity, inflammatory diseases, and transplant rejection. Therapeutic strategies include blocking of costimulation using monoclonal antibodies to the ligand or to the receptor of a costimulatory pair, or using soluble fusion proteins composed of the costimulatory receptor that may bind and block its appropriate ligand. Another approach is induction of co-inhibition using soluble fusion protein of an inhibitory ligand. These approaches rely, at least partially, on the eventual deletion of auto- or allo-reactive T cells (which are responsible for the pathogenic processes in autoimmune diseases or transplantation, respectively), presumably because in the absence of costimulation (which induces cell survival genes) T cells become highly susceptible to induction of apoptosis. Thus, novel agents that are capable of modulating costimulatory signals, without compromising the immune system's ability to defend against pathogens, are highly advantageous for treatment and prevention of such pathological conditions.

Costimulatory pathways play an important role in tumor development. Interestingly, tumors have been shown to evade immune destruction by impeding T cell activation through inhibition of co-stimulatory factors in the B7-CD28 and TNF families, as well as by attracting regulatory T cells, which inhibit anti-tumor T cell responses (see Wang (2006), “Immune Suppression by Tumor Specific CD4⁺ Regulatory T cells in Cancer”, Semin. Cancer. Biol. 16:73-79; Greenwald, et al. (2005), “The B7 Family Revisited”, Ann. Rev. Immunol. 23:515-48; Watts (2005), “TNF/TNFR Family Members in Co-stimulation of T Cell Responses”, Ann. Rev. Immunol. 23:23-68; Sadum, et al., (2007) “Immune Signatures of Murine and Human Cancers Reveal Unique Mechanisms of Tumor Escape and New Targets for Cancer Immunotherapy”, Clin. Canc. Res. 13(13): 4016-4025). Such tumor expressed co-stimulatory molecules have become attractive cancer biomarkers and may serve as tumor-associated antigens (TAAs). Furthermore, costimulatory pathways have been identified as immunologic checkpoints that attenuate T cell dependent immune responses, both at the level of initiation and effector function within tumor metastases.

However, while monotherapy with anti-checkpoint inhibitor antibodies have shown promise, a number of studies (Ahmadzadeh et al., Blood 114:1537 (2009), Matsuzaki et al., PNAS 107(17):7875-7880 (2010), Fourcade et al., Cancer Res. 72(4):887-896 (2012) and Gros et al., J. Clinical Invest. 124(5):2246 (2014)) examining tumor-infiltrating lymphocytes (TILs) have shown that TILs commonly express multiple checkpoint receptors. Moreover, it is likely that TILs that express multiple checkpoints are in fact the most tumor-reactive. In contrast, non-tumor reactive T cells in the periphery are more likely to express a single checkpoint. Checkpoint blockade with monospecific full-length antibodies is likely nondiscriminatory with regards to de-repression of tumor-reactive TILs versus autoantigen-reactive single expressing T cells that are assumed to contribute to autoimmune toxicities.

One target of interest is PVRIG. PVRIG, also called Poliovirus Receptor Related Immunoglobulin Domain Containing Protein, Q6DKI7 or C7orf15, is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). PVRIG binds to Poliovirus receptor-related 2 protein (PVLR2, also known as nectin-2, CD112 or herpesvirus entry mediator B, (HVEB) a human plasma membrane glycoprotein), the binding partner of PVRIG.

Another target of interest is TIGIT. TIGIT is a coinhibitory receptor that is highly expressed on effector & regulatory (Treg) CD4+ T cells, effector CD8+ T cells, and NK cells. TIGIT has been shown to attenuate immune response by (1) direct signaling, (2) inducing ligand signaling, and (3) competition with and disruption of signaling by the costimulatory receptor CD226 (also known as DNAM-1). TIGIT signaling has been the most well-studied in NK cells, where it has been demonstrated that engagement with its cognate ligand, poliovirus receptor (PVR, also known as CD155) directly suppresses NK cell cytotoxicity through its cytoplasmic ITIM domain. Knockout of the TIGIT gene or antibody blockade of the TIGIT/PVR interaction has shown to enhance NK cell killing in vitro, as well as to exacerbate autoimmune diseases in vivo. In addition to its direct effects on T- and NK cells, TIGIT can induce PVR-mediated signaling in dendritic or tumor cells, leading to the increase in production of anti-inflammatory cytokines such as IL10. In T-cells TIGIT can also inhibit lymphocyte responses by disrupting homodimerization of the costimulatory receptor CD226, and by competing with it for binding to PVR.

TIGIT is highly expressed on lymphocytes, including Tumor Infiltrating Lymphocytes (TILs) and Tregs, that infiltrate different types of tumors. PVR is also broadly expressed in tumors, suggesting that the TIGIT-PVR signaling axis may be a dominant immune escape mechanism for cancer. Notably, TIGIT expression is tightly correlated with the expression of another important coinhibitory receptor, PD1. TIGIT and PD1 are co-expressed on the TILs of numerous human and murine tumors. Unlike TIGIT and CTLA4, PD1 inhibition of T cell responses does not involve competition for ligand binding with a costimulatory receptor.

Accordingly, combination therapies utilizing anti-PVRIG antibodies and anti-TIGIT antibodies, which when combined are capable of targeting both pathways, are an attractive combination for antibody combination therapies. Such antibodies will allow for targeting of multiple checkpoint receptors and provide therapeutic importance in the treatment of cancer. As such, anti-PVRIG antibodies and anti-TIGIT antibodies are provide for such combined use as described herein.

### II. BRIEF SUMMARY OF THE INVENTION

Accordingly, the present invention provides an anti-PVRIG and.or anti-TIGIT antibodies that monovalently binds a human PVRIG and monovalently binds TIGIT for use in activating NK cells for the treatment of cancer.

The present invention provides a method of activating NK-cells comprising administering an anti-PVRIG and anti-TIGIT antibody, wherein administering the combination of an anti-PVRIG and anti-TIGIT antibody results in increased activation of NK-cells, optionally as compared to the level of NK-cell activation exhibited for individual administration of an anti-PVRIG or an anti-TIGIT antibody and/or as compared to a control or standard level of NK-cell activation and/or as compared to unactivated NK-cells level.

In some embodiments, the NK-cell activation finds use for the treatment of cancer.

In some embodiments, the anti-PVRIG antibody binds a human PVRIG, and wherein the anti-TIGIT antibody binds human TIGIT.

In some embodiments, the NK-cell activation when both an anti-PVRIG and anti-TIGIT antibody are administered is one-fold, two-fold, three-fold, four-fold, five-fold, or more as compared to the level of NK-cell activation exhibited for individual administration of an anti-PVRIG or an anti-TIGIT antibody, and/or as compared to a control or standard level of NK-cell activation, and/or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG or an anti-TIGIT antibody, and/or as compared to a control or standard level of NK-cell activation, and/or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody, and/or as compared to a control or standard level of NK-cell activation, and/or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody, and/or as compared to a control or standard level of NK-cell activation, and/or as compared to unactivated NK-cells level, wherein PVRL2 is expressed on the cancer cells of the individual to which the anti-PVRIG and anti-TIGIT antibodies are being administered.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-TIGIT antibody, and/or as compared to a control or standard level of NK-cell activation, and/or as compared to unactivated NK-cells level.

9 In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-TIGIT antibody, and/or as compared to a control or standard level of NK-cell activation, and/or as compared to unactivated NK-cells level, wherein PVR is expressed on the cancer cells of the individual to which the anti-PVRIG and anti-TIGIT antibodies are being administered.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody, and/or as compared to a control or standard level of NK-cell activation, and/or as compared to unactivated NK-cells level, wherein PVRL2 is expressed on the cancer cells of the individual to which the anti-PVRIG and anti-TIGIT antibodies are being administered.

In some embodiments, the NK-cells exhibit increased cytotoxicity when both an anti-PVRIG and anti-TIGIT antibody are administered.

In some embodiments, the NK-cell increased cytotoxicity when both an anti-PVRIG and anti-TIGIT antibody are administered is one-fold, two-fold, three-fold, four-fold, five-fold, or more as compared to the level of NK-cell cytotoxicity exhibited for individual administration of an anti-PVRIG or an anti-TIGIT antibody.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG or an anti-TIGIT antibody.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG antibody.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG antibody, wherein PVRL2 is expressed on the cancer cells of the individual to which the anti-PVRIG and anti-TIGIT antibodies are being administered.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-TIGIT antibody.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-TIGIT antibody, wherein PVR is expressed on the cancer cells of the individual to which the anti-PVRIG and anti-TIGIT antibodies are being administered.

In some embodiments, the NK-cell activation is measured based on an increase in proliferation of at least a subset of NK-cells.

In some embodiments, the NK-cell activation is measured by increase in expression of activation markers.

In some embodiments, the activation markers include CD69, CD107a, granzyme, and/or perforin.

In some embodiments, the NK-cell activation is measured based on an increase in immunostimulatory activity.

In some embodiments, the NK-cell activation is measured based on an increase in cytokine secretion.

In some embodiments, the cytokines include IFNγ and/or TNF.

In some embodiments, the NK-cell activation is measured based on an increase in direct killing of target cells by NK-cells in vitro.

In some embodiments, the NK-cell activation is measured based on an increase in direct killing of target cells by NK-cells in vivo.

In some embodiments, the NK-cell activation is measured based on cell surface receptor expression of CD25.

In some embodiments, the anti-PVRIG antibody comprises:
a. a heavy chain variable domain comprising a vhCDR1, vhCDR2, and vhCDR3 from an anti-PVRIG antibody; and
b. a light chain variable domain comprising a vlCDR1, vlCDR2, and vlCDR3 from an anti-PVRIG antibody;
wherein the anti-PVRIG antibody in a) and b) is selected from the group consisting of CHA.7.518.4, CHA.7.518.1, CHA.7.518, CHA.7.524 CHA.7.530, CHA.7.538_1, CHA.7.538_2, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549,CHA.7.550, CHA7.538.1.2, CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019,CPA.7.022, CPA.7.023, CPA.7.024, CPA.7.033, CPA.7.034, CPA.7.036, CPA.7.040, CPA.7.046, CPA.7.047, CPA.7.049, CPA.7.050, CHA.7.518, and the antibodies as depicted in FIGS. 24A-24D and 61A-61P.

In some embodiments, the anti-TIGIT antibody comprises:
a. a heavy chain variable domain comprising a vhCDR1, vhCDR2, and vhCDR3 from an anti-TIGIT antibody; and
b. a light chain variable domain comprising a vlCDR1, vlCDR2, and vlCDR3 from an anti-TIGIT antibody;
wherein the anti-TIGIT antibody in a) and b) is selected from the group consisting of CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8, the antibodies as depicted in FIGS. 23A-23EE and 62A-62FI.

In some embodiments, the PVRIG antibody comprises the vlCDR1, vlCDR2, vlCDR3, vhCDR1, vhCDR2, and vhCDR3 from CHA.7.518.1.H4(S241P) and the TIGIT antibody comprises the vlCDR1, vlCDR2, vlCDR3, vhCDR1, vhCDR2, and vhCDR3 from CPA.9.086.H4(S241P).

In some embodiments, the PVRIG antibody is CHA.7.518.1.H4(S241P) and the TIGIT antibody is CPA.9.086.H4(S241P).

In some embodiments, the anti-PVRIG antibody and/or the anti-TIGIT comprises:
a. a heavy chain comprising VH-CH1-hinge-CH2-CH3; and
b. a light chain comprising VL-CL, wherein the CL is the constant domain of either a kappa or lambda antibody.

In some embodiments, the CL is kappa.

In some embodiments, the CL is lambda.

In some embodiments, the anti-PVRIG antibody and/or the anti-TIGIT antibody is a humanized antibody.

In some embodiments, the cancer is selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS).

In some embodiments, the cancer is selected from the group consisting of triple negative breast cancer, stomach (gastric) cancer, Acute myeloid leukemia (AML), lung cancer (small cell lung, non-small cell lung), Merkel Cells cancer, MSI-high cancer, KRAS mutant tumors, adult T-cell leukemia/lymphoma, myeloma, and Myelodysplastic syndromes (MDS).

In some embodiments, the cancer is selected from the group consisting of advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

In some embodiments, the cancer is AML.

In some embodiments, the individual has AML cancer cells that are PVRL2^{hi}PVR^{low} and/or PVRL2⁺PVR^{low}.

In some embodiments, the AML cancer cells are PVRL2^{hi}PVR^{low} and/or PVRL2⁺PVR^{low}.

In some embodiments, the AML cancer cells are AML blasts.

In some embodiments, the AML is selected from the group consisting of AML with minimal differentiation (M0), AML without maturation (M1), AML with maturation (M2), Acute Promeyelocitic Leukemia (M3), Acute myelomonocytic leukemia (M4), Acute monoblastic/monocytic leukemia (M5a/b), Acute Erythroleukemia (M6), Acute Megakaryocytic Leukemia (M7), Acute basophilic leukemia, Acute panmyelosis with myelofibrosis, therapy related AML (Alkylating agent related AML or Topoisomerase II inhibitor related), AML with myelodysplasia related changes (AMLMRC), AML with myelodysplasia related changes, myeloid sarcoma, myeloid proliferations related to Down syndrome (transient abnormal myelopoeisis or myeloid leukemia associated with Down syndrome), blastic plasmacytoid dentritic cell neoplasm, acute leukemia of ambiguous lineage, and AML with recurrent genetic abnormalities.

In some embodiments, the acute leukemia of ambiguous lineage is selected from the group consisting of acute undifferentiated leukemia, mixed phenotype acute leukemia with t(9;22)(q34;q11.2) (BCR-ABL1), mixed phenotype acute leukemia with t(v;11q23) (MLL rearranged), mixed phenotype acute leukemia (B/myeloid, NOS), mixed phenotype acute leukemia (T/myeloid, NOS), mixed phenotype acute leukemia (NOS, rare types), and other acute leukemia of ambiguous lineage.

In some embodiments, the AML with recurrent genetic abnormalities is selected from the group consisting of AML with t(8;21)(q22;q22) (RUNX1-RUNX1T1), AML with inv(16)(p13.1;q22) or t(16;16)(p13.1;q22) (CBF&beta-MYH11), Acute promyelocytic leukemia with t(15;17)(q22;q12) (PML/RAR&alpha and variants), AML with t(9;11)(p22;q23) (MLLT3-MLL), AML with t(6;9)(p23;q34) (DEK-NUP214), AML with inv(3)(q21q26.2) or t(3;3)(q21;q26.2) (RPN1-EVI1), AML (megakaryoblastic) with t(1;22)(p13;q13) (RBM15-MKL1), AML with mutated NPM1, and AML with mutated CEBPA.

In some embodiments, the AML is related to specific mutations in one or more genes that are selected from the group consisting of FLT3, NPM1, IDH1/2, DNMT3A, KMT2A, RUNX1, ASXL, and TP53.

### III. BRIEF DESCRIPTION OF THE DRAWINGS

FIG. 1 depicts the sequences of human PVRIG (showing two different methionine starting points as well as the full length sequence). The signal peptide is underlined, the ECD is double underlined. PVRIG, also called Poliovirus Receptor Related Immunoglobulin Domain Containing Protein, Q6DKI7 or C7orf15, relates to amino acid and nucleic acid sequences shown in RefSeq accession identifier NP_076975, shown in FIG. 1.

FIG. 2 depicts the sequence of the human Poliovirus receptor-related 2 protein (PVLR2, also known as nectin-2, CD112 or herpesvirus entry mediator B, (HVEB)), the binding partner of PVRIG. PVLR2 is a human plasma membrane glycoprotein.

FIG. 3A-3C shows the CDR sequences for Fabs that were determined to successfully block interaction of the PVRIG with its counterpart PVRL2.

FIGS. 4A-4AA shows the amino acid sequences of the variable heavy and light domains, the full length heavy and light chains, and the variable heavy and variable light CDRs for the enumerated human CPA anti-PVRIG sequences of the invention that both bind PVRIG and block binding of PVRIG and PVLR2.

FIGS. 5A-5H depicts the amino acid sequences of the variable heavy and light domains, the full length heavy and light chains, and the variable heavy and variable light CDRs for eight human CPA anti-PVRIG sequences of the invention that bind PVRIG and but do not block binding of PVRIG and PVLR2.

FIGS. 6A-6G depicts the CDRs for all CPA anti-PVRIG antibody sequences that were generated that bind PVRIG, including those that do not block binding of PVRIG and PVLR2.

FIGS. 7A-7AF depicts the variable heavy and light chains as well as the vhCDR1, vhCDR2, vhCDR3, vlCDR1, vlCDR2 and vlCDR3 sequences of each of the enumerated CHA antibodies for use with the invention, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549,CHA.7.550, and CHA.7.518.4 (these include the variable heavy and light sequences from mouse sequences (from Hybridomas).

FIGS. 8A-8D depicts the vhCDR1, vhCDR2, vhCDR3, vlCDR1, vlCDR2 and vlCDR3 sequences of each of the enumerated CPA antibodies for use with the invention, CPA.7.001 to CPA.7.050 are human sequences (from Phage display).

FIGS. 9A-9C depicts the sequences of human IgG1, IgG2, IgG3 and IgG4.

FIG. 10 depicts a number of human PVRIG ECD fragments.

FIGS. 11A-11I depicts a collation of the humanized sequences of five CHA antibodies.

FIGS. 12A-12E depicts a collation of the humanized sequences of five CHA antibodies.

FIG. 13 depicts schemes for combining the humanized VH and VL CHA antibodies of FIGS. 11A-11I and FIGS. 12A-12E. The “chimVH” and “chimVL” are the mouse variable heavy and light sequences attached to a human IgG constant domain.

FIGS. 14A and 14B depict the variable heavy and light chains as well as the vhCDR1, vhCDR2, vhCDR3, vlCDR1, vlCDR2 and vlCDR3 sequences of each of the enumerated CHA antibodies for use with the invention, CHA.7.518.1.H4(S241P), and CHA.7.538.1.2.H4(S241P).

FIG. 15A-15E depict four humanized sequences for each of CHA.7.518, CHA.7.524, CHA.7.530, CHA.7.538_1 and CHA.7.538_2. All humanized antibodies comprise the H4(S241P) substitution. Note that the light chain for CHA.7.538_2 is the same as for CHA.7.538_1. The “H1” of each is a “CDR swap” with no changes to the human framework. Subsequent sequences alter framework changes shown in larger bold font. CDR sequences are noted in bold. CDR definitions are AbM from website www.bioinf.org.uk/abs/. Human germline and joining sequences from IMGT® the international ImMunoGeneTics® information system www.imgt.org (founder and director: Marie-Paule Lefranc, Montpellier, France). Residue numbering shown as sequential (seq) or according to Chothia from website www.bioinf.org.uk/abs/(AbM). “b” notes buried sidechain; “p” notes partially buried; “i” notes sidechain at interface between VH and VL domains. Sequence differences between human and murine germlines noted by asterisk (*). Potential additional mutations in frameworks are noted below sequence. Potential changes in CDR sequences noted below each CDR sequence as noted on the figure (# deamidation substitutions: Q/S/A; these may prevent asparagine (N) deamidation. @ tryptophan oxidation substitutions: Y/F/H; these may prevent tryptophan oxidation; @ methionine oxidation substitutions: L/F/A).

FIG. 16A to 16C depicts a collation of the humanized sequences of three CHA antibodies: CHA.7.518, CHA.7.538.1, and CHA.7.538.2.

FIG. 17 depicts schemes for combining the humanized VH and VL CHA antibodies. The “chimVH” and “chimVL” are the mouse variable heavy and light sequences attached to a human IgG constant domain.

FIG. 18. Sequence alignment of PVRIG orthologs. Aligned sequences of the human, cynomolgus, marmoset, and rhesus PVRIG extra-cellular domain. The differences between human and cynomolgus are highlighted in yellow.

FIG. 19A-19D depict the amino acid sequences and the nucleic acid sequence for the variable heavy chain (A and B, respectfully) and the amino acid sequences and the nucleic acid sequence for the variable light chain (C and D, respectfully) for AB-407 (BOJ-5 G4-F4).

FIGS. 20A and 20B depicts the amino acid sequences of the constant domains of human IgG1 (with some useful amino acid substitutions), IgG2, IgG3, IgG4, IgG4 with a hinge variant that finds particular use in the present invention, and the constant domains of the kappa and lambda light chains.

FIG. 21 depicts the sequences of human and cynomolgus macaque (referred to as cyno) TIGIT ECD and of the human PVR ECD proteins.

FIG. 22A-22D depict the sequences of anti-TIGIT antibodies. Unless otherwise noted, the CDRs utilize the IMGT numbering (including the antibodies of the sequence listing).

FIG. 23A-23EE depict the sequences of numerous anti-TIGIT antibodies for use in the present invention.

FIG. 24A-24D provides additional anti-PVRIG antibodies for use in the the present invention.

FIG. 25. Receptor-ligand interactions in the DNAM-1/TIGIT/PVRIG axis.

FIG. 26. A-F) Healthy donor PBMCs were co-cultured with A-C) SKBR3 or D-F) KG1a in the presence of the indicated blocking antibodies. A,D) Lysis of target cells and expression of B,E) CD69 and C,F) CD107a on NK cells was assessed after 4 hr. G) Expression of PVRL2 or PVR (red) on SKBR3 and KG1a cells compared with isotype control stain (grey).

FIG. 27. A-C) Expression of A) PVRL2 B) PVR or C) PVRIG on blasts or immune cell types in the bone marrow of AML patients (n=19-20) or healthy donors (n=13). D-F) Representative histograms of D) PVRL2 on AML blasts E) PVR on AML blasts or F) PVRIG on NK cells in the bone marrow of an AML patient. Histograms of test (red) and isotype control stains (blue) are shown.

FIG. 28. Expression of A-C) PVRIG D-F) TIGIT or G-I) DNAM-1 on isolated NK cells after 24 hr co-culture with tumour cells, or 24 hr stimulation with the indicated cytokines or agonistic antibodies. Percentage change in MFI relative to NK alone is shown.

FIG. 29. Expression of A,C,D) PVRIG and B) CD69 on isolated NK cells incubated alone, with K562 cells, or with plate-bound α-CD16 antibody at 37° C. for the indicated time points, in the presence or absence of monensin (mon) or brefeldin A (BFA).

FIG. 30. Modulation of DNAM-1/TIGIT/PVRIG on NK cells upon activation.

FIG. 31. Blockade of PVRIG enhanced NK cell killing of tumour cell lines. (A) Percentage lysis of KG1a cells after 4 hr co-culture with PBMCs in the presence of anti-PVRIG, anti-TIGIT, anti-PVRIG+anti-TIGIT, or isotype antibodies, measured by ⁵¹Cr release assay. Representative data (mean±SD of triplicates) of 4 experiments shown. (B) NK:target ratio required to achieve 10% lysis, determined by non-linear regression of curves plotted as in A. Each symbol represents an individual donor, n=4. NK cell expression of (C) CD69 and (D) CD107a after 4 hr co-culture of PBMCs with KG1a (8:1 E:T ratio) in the presence of the indicated blocking antibodies or isotype control antibody. Representative data (mean±SD of triplicates) of 2 experiments is shown. (E) Percentage lysis of KG1a cells after 4 hr co-culture with PBMCs in the presence of anti-PVRIG or isotype antibodies, with or without 4 mM EGTA. Representative data (mean±SD of triplicates) of 2 experiments is shown. (F) Percentage lysis of SKBR3 cells after 4 hr co-culture with PBMCs in the presence of anti-PVRIG, anti-TIGIT, anti-PVRIG+anti-TIGIT, or isotype antibodies, measured by ⁵¹Cr release assay. Representative data (mean±SD of triplicates) of 3 experiments is shown. (G) NK:target ratio required to achieve 10% lysis, determined by non-linear regression of curves plotted as in F. Each symbol represents an individual donor (n=3). NK cell expression of (H) CD69 and (I) CD107a after 4 hr co-culture of PBMCs with SKBR3 (2:1 E:T ratio) in the presence of the indicated blocking antibodies or isotype control antibody. Representative data (mean±SD of triplicates) of 2 experiments is shown. (J) Expression of PVRL2 and PVR (red histograms) on SKBR3 and KG1a cells compared with isotype control stain (grey histograms). NK:target ratios in (A, E, F) were calculated using % of NK cells in PBMC determined by flow cytometry. Significance was determined by repeated measures one-way ANOVA (A, B, F, G) or one-way ANOVA (C, D, H, I) with Holm-Sidak's multiple comparisons test, ns p>0.05, * p<0.05, ** p<0.01, *** p<0.001, **** p<0.0001.

FIG. 32. PVRIG and its ligand PVRL2 are expressed in AML patient bone marrow. Expression of (A) PVRL2 (B) PVR or (C) PVRIG on blasts or immune cell types in the bone marrow of AML patients (n=19-20) or healthy donors (n=13). Open triangles mark the patient shown in D-F, blue triangles mark the patient used for G-H. Representative histograms of (D) PVRL2 and (E) PVR on AML blasts, or (F) PVRIG on NK cells in the bone marrow of an AML patient. Overlay histograms of test (red) and isotype control stains (grey) are shown. NK cell expression of (G, I, K) CD69 and (H, J, L) CD107a after 4 hr co-culture of healthy donor PBMCs with AML patient bone marrow (8:1 E:T ratio) in the presence of the indicated blocking antibodies or isotype control antibody (mean±SD of triplicates, n=3 patients). Significance was determined by one-way ANOVA with Holm-Sidak's multiple comparisons test, ns p>0.05, * p<0.05, ** p<0.01, ***p<0.001, **** p<0.0001.

FIG. 33. PVRIG expression on NK cells is decreased upon activation. (A) PVRIG expression on isolated NK cells after 24 hr cultured alone, or co-cultured with K562 or KG1a cells (1:1 ratio). Shown is percentage change in PVRIG MFI relative to NK alone, each point represents an individual donor (n=3), bars represent mean±SD. (B) PVRIG expression on isolated NK cells after 24 hr incubation alone or in the presence of 100 U/ml IL-2 and 10 ng/ml IL-12. Shown is percentage change in PVRIG MFI relative to NK alone, each point represents an individual donor (n=3), bars represent mean±SD. (C) Expression of PVRIG and CD69 on isolated NK cells after 24 hr incubation with 25 U/ml IL-2, 100 U/ml IL-2, or combinations of IL-2 (100 U/ml), IL-12 (10 ng/ml), IL-15 (50 ng/ml) and IL-18 (50 ng/ml), as indicated. Representative data of 2 independent experiments is shown. Expression of (D) CD69 or (E) PVRIG on isolated NK cells after 24 hr incubation with the indicated plate-bound antibodies. Shown is percentage change in PVRIG MFI relative to isotype. Each donor is represented by a distinct symbol (n=3), bars represent mean±SD. (F) Expression of PVRIG vs CD69 on isolated NK cells after 24 hr incubation with indicated stimuli, as in (C) and (E). Significance determined by one-way ANOVA with Holm-Sidak's multiple comparisons test (A, D, E) or Student's t-test (B), ns p>0.05, * p<0.05, ** p<0.01, **** p<0.0001.

FIG. 34. Modulation of DNAM-1 and TIGIT expression on NK cells upon activation. (A-C) TIGIT or (D-F) DNAM-1 expression on isolated NK cells after 24 hr culture with (A, D) K562 or KG1a cells (1:1 ratio); (B, E) 100 U/ml IL-2 and 10 ng/ml IL-12; or (C, F) with the indicated plate-bound antibodies. Percentage change in MFI relative to NK alone is shown, each point represents an individual donor (n=3), bars represent mean±SD. Significance determined by one-way ANOVA with Holm-Sidak's multiple comparisons test (A, C, D, F) or Student's t-test (B, E), ns p>0.05, * p<0.05, ** p<0.01, *** p<0.001, **** p<0.0001. (G) Model of PVRIG-TIGIT-DNAM-1 modulation upon NK cell activation. Left: Upon recognition of tumour cells expressing PVR and PVRL2, NK cells increase TIGIT but lose expression of both PVRIG and DNAM-1. This loss of DNAM-1 may result from a tumour-intrinsic mechanism of immune escape, whereby tumour cells expressing DNAM-1 ligands induce loss of DNAM-1 expression on immune cells. Right: Upon activation via cytokines such as IL-2 and IL-12, or by stimulation of activating receptors such as CD16, NK cells decrease expression of PVRIG while increasing expression of TIGIT and DNAM-1. The increased expression of DNAM-1 relative to the decreased expression of PVRIG may serve to push the balance within the cell towards more activating signalling. In this way, PVRIG may act to constitutively dampen NK responsiveness in the steady state and is lost upon activation to lower the activation threshold of the NK cell.

FIG. 35. Differential regulation of surface and intracellular PVRIG by CD56^{dim} and CD56^{bright} NK cells. (A) PVRIG expression on isolated NK cells (gated on CD56^{dim} or CD56^{bright} subsets) after 24 hr stimulation with 100 U/ml IL-2 and 10 ng/ml IL-12, measured by surface or total (intracellular+surface) staining. Representative of 2-3 experiments. (B, C) Expression of surface PVRIG on isolated NK cells (gated on CD56^{dim} or CD56^{bright}) after 24 hr culture with (B) 100 U/ml IL-2 and 10 ng/ml IL-12; or (C) with K562 or KG1a cells (1:1 ratio). Shown is percentage change in MFI relative to NK alone, bars represent mean±SD of 3 experiments. PVRIG expression on NK cells gated on (D) CD56^{dim} or (E) CD56^{bright} subsets, measured by surface or total (intracellular+surface) staining after 24 hr stimulation with 100 U/ml IL-2 and 10 ng/ml IL-12. Shown is percentage change in PVRIG MFI relative to NK alone, bars represent mean±SD of 2-3 experiments. Significance determined by Student's t-test, ns p>0.05, * p<0.05, ** p<0.01.

FIG. 36. PVRIG is constitutively trafficked to the NK cell surface. (A) PVRIG and (B) CD69 expression on isolated NK cells incubated alone, with K562 cells (1:1 ratio), or with plate-bound anti-CD16 antibody at 37° C. for the indicated timepoints. Representative data (mean±SD of duplicates) of 2 experiments is shown. (C, D) PVRIG expression on isolated NK cells incubated either (C) alone or (D) with plate-bound anti-CD16 antibody at 37° C. for the indicated timepoints, in the presence or absence of monensin (mon) or brefeldin A (BFA). Representative data (mean±SD of duplicates) of 2 experiments is shown. Significance determined by multiple t-tests with Holm-Sidak's correction, * p<0.05 compared with NK alone (A, B) or untreated (C, D).

FIG. 37. Antibodies used for flow cytometry.

FIG. 38. AML patient clinical characteristics. NA: data not available; M0: AML with minimal differentiation; M1: AML without maturation; M2: AML with maturation; M4: Acute myelomonocytic leukemia; M5a/b: Acute monoblastic/monocytic leukemia; AMLT: therapy related AML; AMLMRC: AML with myelodysplasia related changes.

FIG. 39. Expression of PVRIG and TIGIT on NK cells from healthy donors. (A) PVRIG and (B) TIGIT expression on NK cells from healthy donor PBMCs. Histograms of test (red) and isotype control stains (grey) from four representative donors are shown (each row is one donor).

FIG. 40. Relative capacity of PVRIG or TIGIT blockade to enhance NK cell killing is related to target cell PVR expression. (A) Expression of PVRL2 and PVR (red histograms) on AML-193, Kasumi-1, ML-2 and THP-1 cells compared with isotype control stain (grey histograms). Percentage lysis of (B) AML-193, (C) Kasumi-1, (D) ML-2 or (E) THP-1 cells after 4 hr co-culture with PBMCs in the presence of anti-PVRIG, anti-TIGIT, or isotype antibodies, measured by 51Cr release assay. Each graph shows mean±SD of triplicates for an individual healthy donor (donor ID labelled above each graph), with NK:target ratios calculated using % of NK cells in PBMC as determined by flow cytometry. Significance determined by Student's t-test, * p<0.05, ** p<0.01, *** p<0.01; red=anti-PVRIG vs iso, green=anti-TIGIT vs iso.

FIG. 41. Gating strategy for AML bone marrow. AML blasts were identified as CD45loSSCint, along with various combinations of the markers CD33, CD34, CD117, depending on the patient. Mature myeloid cells (CD45hiSSChi) could be further subdivided into CD14+CD11b+ or CD14-CD11b+ cells. The CD14-CD11b+ subset was not present in all patients, and was absent in all healthy donors, therefore was not included in further analysis. Within the lymphoid population (CD45hiSSClo), NK cells (CD56+CD3-), NKT cells (CD56+CD3+), CD8+ T cells (CD3+CD56-CD8+) and CD8− T cells (CD3+CD56-CD8-) could be identified. For healthy donor BM, the same gating strategy was employed, with CD45loSSCint immature myeloid cells used as an analogous population to compare with AML blasts.

FIG. 42. No correlation between expression of PVRIG or PVRL2 in AML patient bone marrow and AML subtype or blast percentage. Expression of (A) PVRIG on NK cells or (B) PVRL2 on blasts in AML patients coloured by disease subtype (WHO classification; AMLT: therapy related acute myeloid leukemia, AMLMRC: acute myeloid leukemia with myelodysplasia related changes). Pearson correlation between (C) NK cell PVRIG expression or (D) blast PVRL2 expression with bone marrow blast percentage, or (E) NK cell PVRIG expression with blast PVRL2 expression.

FIG. 43. NK cells are not strongly activated by IL-2 and IL-12 stimulation within 4 hours. (A) PVRIG and (B) CD69 expression on isolated NK cells incubated alone or with 100 U/ml IL-2 and 10 ng/ml IL-12 at 37° C. for the indicated timepoints. Mean±SD of duplicates from 1 experiment is shown. Significance determined by multiple t-tests with Holm-Sidak's correction, * p<0.05 compared with NK alone.

FIG. 44. Expression of TIGIT on cells isolated from dissociated tumors and NATs. A) TIGIT expression is shown on CD8+T, non-Treg CD4+T, Treg CD4+T, and NK cells for each tumor type. Each dot within a column represents an individual sample. Mean±SEM is presented. B) Across all tumor samples examined, the expression of TIGIT on the different cell types is shown. The median is depicted by the middle line and the upper and lower quartiles are depicted by the boxes above and below the median line. The whiskers depict 1.5 times the interquartile range. C) Representative histograms for TIGIT expression (gray) compared to an isotype control (white) are shown for four lymphocyte subsets isolated from an endometrial and a head and neck tumors. D) Matched tumor and NAT samples were assessed for TIGIT expression on CD8⁺ T cells and CD4⁺ Tregs. Each line represents a matched donor.

FIG. 45. Expression of PVR in the TME. A) Representative images from digital image analysis of PVR IHC staining in lung tumor. Staining of Pan-CK and PVR was carried out on serial sections from FFPE tumor samples. Top left: Pan-CK tumor marker staining. Top right: Tumor/stroma boundary classifier. Bottom left: PVR staining within the tumor and adjacent stroma. Bottom right: Cell identification and membrane expression classifier. B) Summary of PVR H-score values from the tumor and stroma of 10 NSCLC, SCLC, TNBC, HCC, and prostate FFPE tumor samples. Each dot represents an individual sample, with tumor expression in black and stroma expression in gray. Mean±SEM is presented.

FIG. 46. CPA.9.086.H4(S241P) binds specifically to TIGIT and blocks TIGIT:PVR binding. A) Binding of CPA.9.086.H4(S241P) to human, cynomolgus, and mouse TIGIT over-expressing cells and non-expressing parental cells. B) Assessment of the ability of CPA.9.086.H4(S241P) and chimeric CPA.9.086.H4(S241P) to block the interaction of TIGIT and PVR. Concentrations shown on x axis are total binding site concentration in nM.

FIG. 47. CPA.9.086.H4(S241P) does not induce ADCC or CDC activity. A) ADCC activity assessment for CPA.9.086.H4(S241P). B) CDC activity assessment for CPA.9.086.H4(S241P). Campath (anti-CD52) hIgG1 was used as a positive control. Representative data from n≥2 is shown for each graph.

FIG. 48. CPA.9.086.H4(S241P) enhances human T cell function. A) CPA.9.086.H4(S241P) increases IL-2 signaling. Representative data (n≥2) shows the RLU (mean±SD) of the luciferase signal from a 6-hour co-culture of Jurkat IL-2-RE luciferase human TIGIT cells and CHO-K1 human PVR cells. A 20 point, 1.5-fold dilution series starting at 133 nM was used for each antibody. B) The expression of PD-1, PVRIG and TIGIT on CMV-reactive CD8⁺ T cells (top row, n=3 donors), and of PVR, PVRL2, PD-L1, and HLA-A2 on Mel-624-pp65 was assessed. White histograms represent the isotype control staining, and gray represent the expression of the target of interest. C) CMV-reactive CD8⁺ T cells were co-cultured with Mel-624-pp65 cells for 18 hours in the presence of 10 μg/mL CPA.9.086.H4(S241P) or hIgG4 isotype control antibody. Percent change in IFN-γ for each condition relative to isotype control is depicted by the number above each bar. The bar graphs show the average±SD. Data were analyzed by paired Student t—test; * p<0.05; ** p<0.01; *** p<0.001. Data shown is representative of n≥3 experiments (n=3 donors). D) CMV-reactive CD8⁺ T cells were co-cultured with CMV peptide-pulsed Mel-624 cells engineered to express hPVR, hPVRL2, and luciferase in the presence of CPA.9.086.H4(S241P) in 2-fold dose-titration range from 6.6-0.006 nM. Percent specific cytotoxicity was calculated by (1−(RLU_{(target cells+ T cells+antibody)}/RLU_{(target cells+ T cells+media alone)}))×100.

FIG. 49. CPA.9.086.H4(S241P) combined with CHA.7.518.1.H4(S241P) or anti-PD1 enhances human lymphocyte function. A) Expression of PVR, PVRL2, PD-L1, and HLA-A2 on Mel-624-pp65 parental and PD-L1 overexpressing cells. B) CPA.9.086.H4(S241P) was added in a 19 point, 2-fold dilution series (66-0.0002 nM) in combination with either a fixed concentration of 10 μg/mL anti-PVRIG, 10 μg/mL anti-PD-1 or 10 μg/mL hIgG4 isotype control to co-culture of CMV-reactive CD8⁺ T cells and Mel pp65-hPD-L1 cells. Percent change in IFN-γ relative to isotype control is shown for each condition. Graphs show representative data from two independent experiments (n=3 donors). C) The expression of TIGIT, PVRIG and CD16 on rhIL-15-primed NK cells (representative example from a healthy donor). White histograms represent the isotype control staining, and gray represent the expression of the target of interest. D) CPA.9.086.H4(S241P), CHA.7.518.1.H4(S241P), CPA.9.086.H4(S241P)+CHA.7.518.1.H4(S241P) or hIgG4 isotype control mAbs were added to NK:CAL-27 cell co-cultures at 10 μg/mL. Following 4 hours, CD107a cell surface expression on NK cells was analyzed by flow cytometry. A representative donor is shown in the left panel. The bar graphs show the average±SD. NK cytotoxicity with four donors are summarized in the right panel. Data were analyzed by paired student t—test.

FIG. 50. Chimeric CPA.9.086.H4(S241P) enhances in-vitro anti-tumor activity of mouse T cells. A) The expression of TIGIT and PD-1 on OT-1 splenocytes activated for 3 days with OVA₍₂₅₇₋₂₆₄₎ peptide and rhIL-2, as well as of H-2K^{b}, PVR, and PD-L1 on MC38 target cells was assessed. Grey histograms represent the target staining and white histograms represent staining with the matched isotype control antibodies. Plots shown are from a representative experiment (n=2). B) Mouse OVA-specific CD8⁺ T cells were co-cultured with OVA-peptide-pulsed MC38 cells in the presence of the chimeric CPA.9.086.H4(S241P) as a monotherapy or in combination with PD-L1 blockade. The secretion of IFN-γ relative to isotype control is shown for each condition. Mean±SD are shown by the hash marks. C). TIGIT expression on infiltrating lymphocytes isolated from s.c. CT26 tumors is shown as fold expression relative to the isotype control antibody (MFIr) for each cell subset. Each dot represents an individual mouse (n=3) and mean±SEM. D) Representative TIGIT expression on indicated cell populations from CT26 tumors. Blue, red, and grey histograms represent staining with CPA.9.086.H4S241P), chimeric CPA.9.086.H4(S241P), and the mIgG1 isotype control, respectively. E) PVR, PVRL2, and P1)-L1 expression on CD45⁻ cells isolated from CT26 tumors. Grey histogram represents mIgG1 isotype control. Representative MFIr values are reported to the right of each histogram.

FIG. 51, Anti-tumor effect of chimeric CPA.9.086.H4(S241P) in the CT26 and Renca tumor models as a single agent and in combination with anti-PVRIG or anti-PD-L1. BALB/c mice were s.c. injected with C126 or Renca cells and dosed with the indicated antibodies starting from day 8 for combination therapies. Tumor volumes represented as the mean volume±SEM and Kaplan-Meier survival curves are shown for the chimeric CPA.9.086.H4(S241P) and anti-PVRIG combination in the CT26 model (A), the chimeric CPA.9.086.H4(S241P) and anti-PD-L1 combination in the C126 model (B), or the chimeric CPA.9.086114(S241P) and anti-PVRIG combination in the Renca model (C). One representative experiment of n≥2 for each combination is shown.

FIG. 52. The expression of PVR on various cell subsets from dissociated bladder, breast, colorectal, endometrial, head and neck, lung, kidney, ovarian, prostate, stomach, and uterine human tumors is shown. Cancer type was sorted by mean PVR expression and depicted from highest (left) to lowest (right). Each dot represents an individual sample. The mean and standard error are depicted by blue and black marks. A) PVR expression on CD14+ macrophages. B) PVR expression on the non-immune CD45− cell subset. C) Average PVR expression on all cell subsets. The median is depicted by the middle line and the upper and lower quartiles are depicted by the boxes above and below the median line. The whiskers depict 1.5 times the interquartile range. D) Representative histograms for PVR expression (blue) compared to an isotype control (red) shown for non-immune CD45− cells and TAMs isolated from an ovarian and lung tumor. Data are generated from panel 2. E) The expression of PVR was scored by pathologists as described in the protocols section using the 0, 1, 2, and 3 scoring system. Representative tumor types shown are breast for Score 0, lymphoma for Score 1, lung for Score 2, and endometrium for Score 3. F) Description of scoring scale for human PVR expression by IHC in FFPE tissues.

FIG. 53. The expression of PVR in 15 different types of tumor tissues (n=8-24 patients per indication) and in normal healthy tissues (n=1-4) is shown.

FIG. 54. A) Cell-based binding of CPA.9.086.H4(S241P) to human, cynomolgus and mouse TIGIT overexpressing cells and non-expressing parental cells. B) Six kinetic exclusion assay (KinExA) replicates of CPA.9.086.H4(S241P) antibody titrated with recombinant hTIGIT protein globally fit to a simple 1:1 equilibrium model to estimate the KD. KD=626 fM (95% CI KD high=988 fM, KD low=350 fM).

FIG. 55. Groups of 10 BALB/c or C57Bl/6 mice were s.c. injected with CT26 (A) or Renca cells (B), respectively. For combination groups, tumors were measured on day 8, and mice were randomized based on tumor size. Mice were then treated with the indicated antibodies (10 mg/kg isotype control mIgG1, 3 mg/kg anti-PD-L1 mIgG1, 10 mg/kg anti-PVRIG mIgG1, or 10 mg/kg chimeric CPA.9.086.H4(S241P) mIgG1) followed by additional doses on days 10, 12, 14, 16, and 18. Individual tumors measurements for each mouse are depicted (n=10 per group).

FIGS. 56A-56F. Human solid and hematological tumor sample histological type and panel information.

FIGS. 57A-57D. Human Tumor Panel 1-4. Antibodies to lineage markers were used according to the manufacturer's recommendation. All isotype control antibodies and target specific antibodies were used at 5 μg/mL final concentration. 1×10⁶ dissociated tumor cells per sample were seeded into a 96-well V-bottomed plate for staining. Samples were first stained with Aqua Live Dead (Life Technologies) to distinguish live cells from dead cells and with a cocktail of anti-CD16 (BioLegend, 3G8), anti-CD32 (Thermofisher, FCGR2), anti-CD64 (BioLegend, 10.1) antibodies to block un-specific binding to Fcγ receptors. Samples were washed twice with FACS buffer (1% BSA, 0.1% sodium azide, in 1×PBS) and stained with a target antibody or control isotype cocktail as above. All staining was carried out for 30 minutes at 4° C. Samples were then washed twice and acquired on a Fortessa X-20 flow cytometer (BD Biosciences). Analysis was completed using FlowJo (TreeStar LLC), with gating on specific populations as specified below (all gated on live cells).

FIG. 58. Definition of specific cell subsets based on lineage marker staining for human tumor panels 1-4.

FIGS. 59A-59B. Human CMV cell phenotyping and human Mel-624-pp65 target cell cocktails. CMV specific human CD8+ T cells were defined as CD14-CD19-CD56-CD3+CD8+CMV tetramer+.

FIGS. 60A-60B. Mouse in-vitro T and target cell phenotyping and mouse ex-vivo TIL cocktails. Ex-vivo TILs were defined as CD45+CD3+CD8+.

FIG. 61A-61P provides additional anti-PVRIG antibodies for use in the the present invention.

FIGS. 62A-62FI provide additional anti-TIGIT antibodies for use in the present invention.

### IV. DETAILED DESCRIPTION OF THE INVENTION

### A. Overview

The present invention provides a number of useful anti-PVRIG and anti-TIGIT, for use in particular in the treatment of cancer based on their ability to enhance NK-cell activation and thus tumor killing. Cancer can be considered as an inability of the patient to recognize and eliminate cancerous cells. In many instances, these transformed (e.g. cancerous) cells counteract immunosurveillance. There are natural control mechanisms that limit T-cell activation in the body to prevent unrestrained T-cell activity, which can be exploited by cancerous cells to evade or suppress the immune response. Restoring the capacity of immune effector cells-especially T cells-to recognize and eliminate cancer is the goal of immunotherapy. The field of immuno-oncology, sometimes referred to as “immunotherapy” is rapidly evolving, with several recent approvals of T cell checkpoint inhibitory antibodies such as Yervoy, Keytruda and Opdivo. These antibodies are generally referred to as “checkpoint inhibitors” because they block normally negative regulators of T cell immunity. It is generally understood that a variety of immunomodulatory signals, both costimulatory and coinhibitory, can be used to orchestrate an optimal antigen-specific immune response. Generally, these antibodies bind to checkpoint inhibitor proteins such as CTLA-4 or PD-1, which under normal circumstances prevent or suppress activation of cytotoxic T cells (CTLs). By inhibiting the checkpoint protein, for example through the use of antibodies that bind these proteins, an increased NK-cell response and/or an increased T cell response against tumors can be achieved. That is, these cancer checkpoint proteins suppress the immune response; when the proteins are blocked, for example using antibodies to the checkpoint protein, the immune system is activated, leading to immune stimulation, resulting in treatment of conditions such as cancer and infectious disease.

The present invention is directed to the use of antibodies to additional checkpoint proteins, PVRIG and TIGIT. PVRIG is expressed on the cell surface of NK and T-cells and shares several similarities to other known immune checkpoints. The identification and methods used to show that PVRIG is a checkpoint receptor are discussed in WO2016/134333, expressly incorporated herein by reference. Anti-PVRIG antibodies to human PVRIG that block the interaction and/or binding of PVLR2 are provided herein. When PVRIG is bound by its ligand (PVRL2), an inhibitory signal is elicited which acts to attenuate the immune response of NK and T-cells against a target cell (i.e. analogous to PD-1/PDL1). Blocking the binding of PVRL2 to PVRIG shuts-off this inhibitory signal of PVRIG and as a result modulates the immune response of NK and T-cells. Utilizing an antibody against PVRIG that blocks binding to PVRL2 is a therapeutic approach that enhances the killing of cancer cells by NK and T-cells. Blocking antibodies have been generated which bind PVRIG and block the binding of its ligand, PVRL2.

Similarly, TIGIT has been shown to also have attributes of a checkpoint receptor, and the present invention provides anti-TIGIT antibodies that block the interaction and/or binding of TIGIT to PVR are provided. When TIGIT is bound by its ligand (PVR), an inhibitory signal is elicited which acts to attenuate the immune response of NK and T-cells against a target cell (i.e. analogous to PD-1/PDL1). Blocking the binding of PVR to TIGIT shuts-off this inhibitory signal of TIGIT and as a result modulates the immune response of NK and T-cells. Utilizing an antibody against TIGIT that blocks binding to PVR is a therapeutic approach that enhances the killing of cancer cells by NK and T-cells. Blocking antibodies have been generated which bind TIGIT and block the binding of its ligand, PVR.

Additionally, the invention provides anti-PVRIG and anti-TIGIT antibodies which can be combined for use in the treatment of cancer.

### B. Definitions

In order that the application may be more completely understood, several definitions are set forth below. Such definitions are meant to encompass grammatical equivalents.

IgG domain definitions used herein are in accordance with IMGT reference sequences (www.IMGT.org)

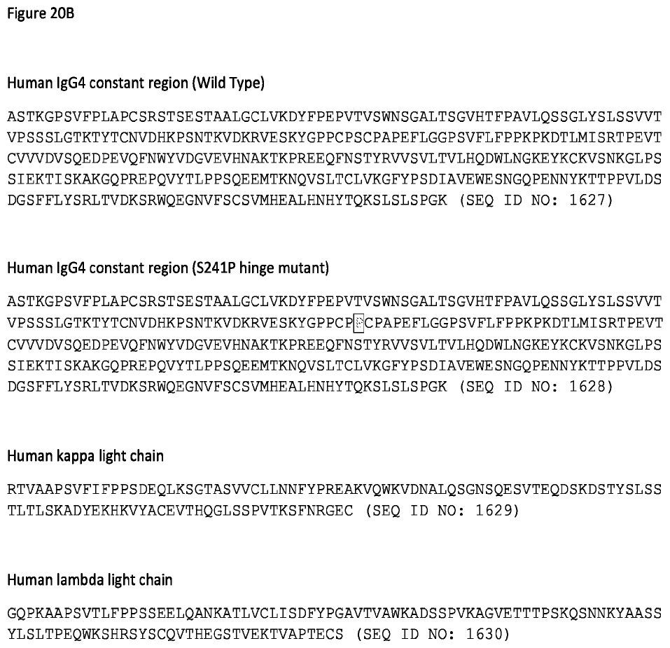

By “ablation” herein is meant a decrease or removal of activity. In some embodiments, it is useful to remove activity from the constant domains of the antibodies. Thus, for example, “ablating FcγR binding” means the Fc region amino acid variant has less than 50% starting binding as compared to an Fc region not containing the specific variant, with less than 70-80-90-95-98% loss of activity being preferred, and in general, with the activity being below the level of detectable binding in a Biacore assay. As shown in FIG. 20A-20B, one ablation variant in the IgG1 constant region is the N297A variant, which removes the native glycosylation site and significantly reduces the FcγRIIIa binding and thus reduces the antibody dependent cell-mediated cytotoxicity (ADCC).

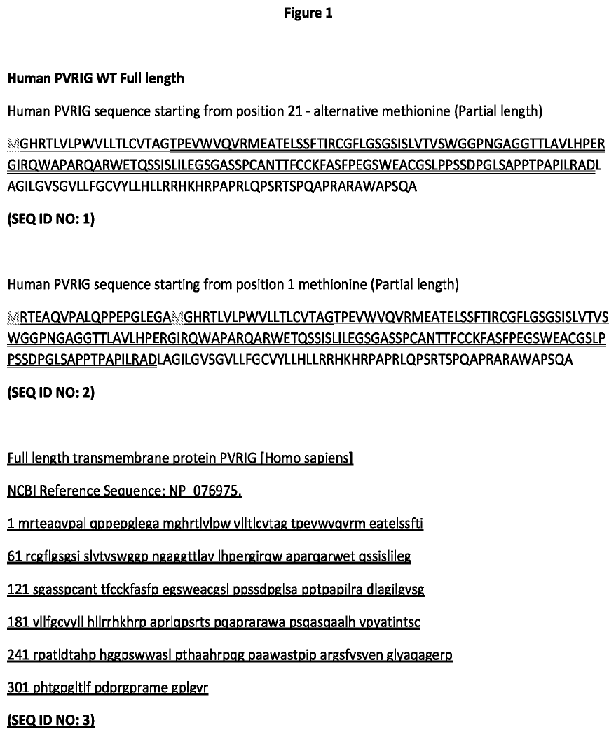

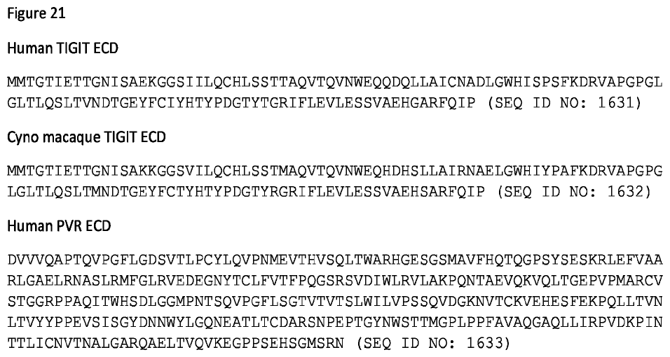

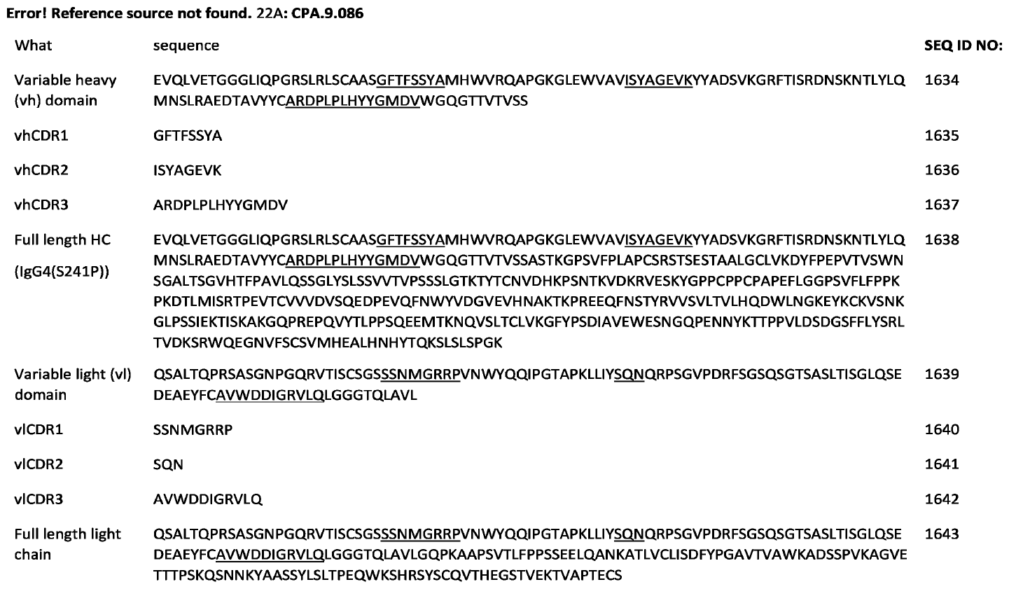

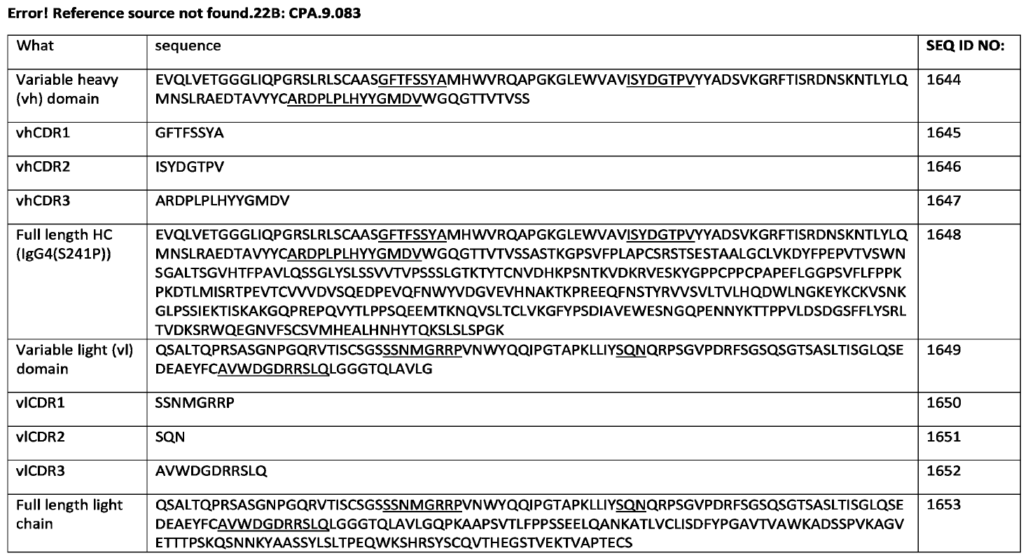

By “antigen binding domain” or “ABD” herein is meant a set of six Complementary Determining Regions (CDRs) that, when present as part of a polypeptide sequence, specifically binds a target antigen as discussed herein. Thus, a “TIGIT antigen binding domain” binds TIGIT antigen (the sequence of which is shown in FIG. 21) as outlined herein. Similarly, a “PVRIG antibody binding domain” binds PVRIG antigen (the sequence of which is shown in FIG. 1) as outlined herein. As is known in the art, these CDRs are generally present as a first set of variable heavy CDRs (vhCDRs or V_{H}CDRs) and a second set of variable light CDRs (vhCDRs or V_{L}CDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and variable light chain. In a “Fab” format, the set of 6 CDRs are contributed by two different polypeptide sequences, the variable heavy domain (vh or V_{H}; containing the vhCDR1, vhCDR2 and vhCDR3) and the variable light domain (vl or V_{L}; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). The phrase “antigen binding portion” can comprise an ABD or be synonymous with ABD.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution N297A refers to a variant polypeptide, in this case an Fc variant, in which the asparagine at position 297 is replaced with alanine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233− or E233 #, E233( ) or E233del designates a deletion of glutamic acid at position 233. Additionally, EDA233- or EDA233 # designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example, S241P or S228P is a hinge variant with the substitution proline at position 228 relative to the parent IgG4 hinge polypeptide, wherein the numbering S228P is according to the EU index and the S241P is the Kabat numbering. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, e.g., “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. For example, homo-phenylalanine, citrulline, ornithine and noreleucine are considered synthetic amino acids for the purposes of the invention, and both D- and L- (R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CH1, VL, and CL immunoglobulin domains. Fab may refer to this region in isolation, or this region in the context of a full length antibody or antibody fragment.

By “Fv” or “Fv fragment” or “Fv region” as used herein is meant a polypeptide that comprises the VL and VH domains of a single antibody. As will be appreciated by those in the art, these generally are made up of two chains.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In general, the linker is a scFv linker as is generally known in the art, with the linker peptide predominantly including the following amino acid residues: Gly, Ser, Ala, or Thr.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification. Similarly, because IgG1 has a proline at position 241 and IgG4 has a serine there, an IgG4 molecule with a S241P is considered an IgG subclass modification. Note that subclass modifications are considered amino acid substitutions herein.

By “non-naturally occurring modification” as used herein is meant an amino acid modification that is not isotypic. For example, because none of the IgGs comprise AN asparagine at position 297, the substitution N297A in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

By “heavy constant region” herein is meant the CH1-hinge-CH2-CH3 portion of an antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. In the present case, one target antigen of interest herein is TIGIT, usually human TIGIT and optionally cyno TIGIT, as defined below. Another target antigen of interest is PVRIG, usually human PVRIG and optionally cyno PVRIG, as defined below.

By “target cell” as used herein is meant a cell that expresses a target antigen.

By “variable region” as used herein is meant the region of an immunoglobulin that comprises one or more Ig domains substantially encoded by any of the Vκ (V.kappa), Vλ (V.lamda), and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁹ M, at least about 10⁻¹⁰ M, at least about 10⁻¹¹ M, at least about 10⁻¹² M, at least about 10⁻¹³ M, at least about 10⁻¹⁴ M, at least about 10⁻¹⁵ M, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using surface plasmon resonance (e.g. Biacore assay) and flow cytometry with antigen-expressing cells.

### C. Sequences

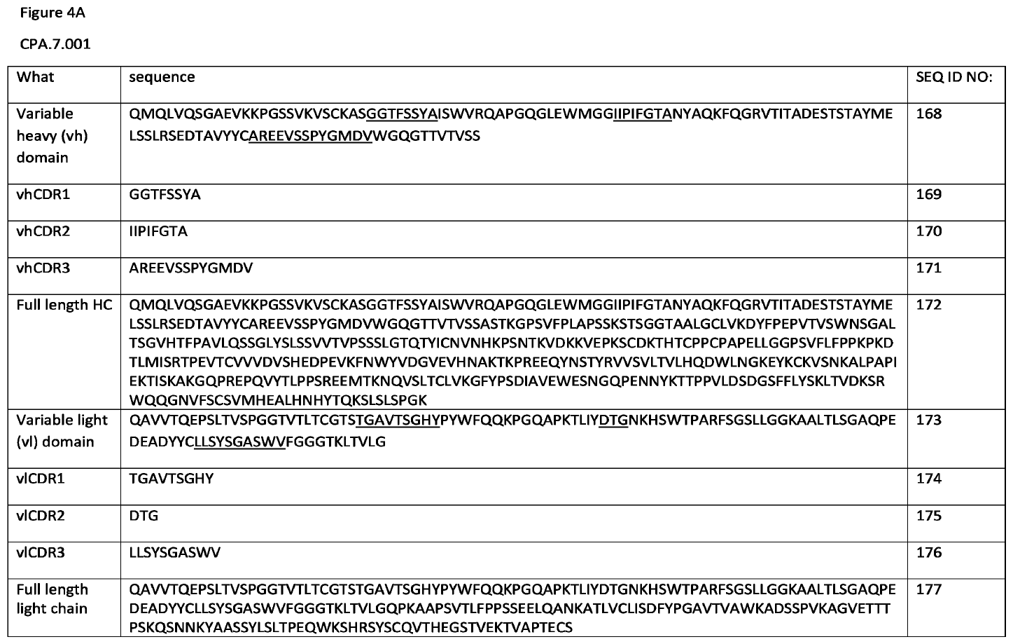

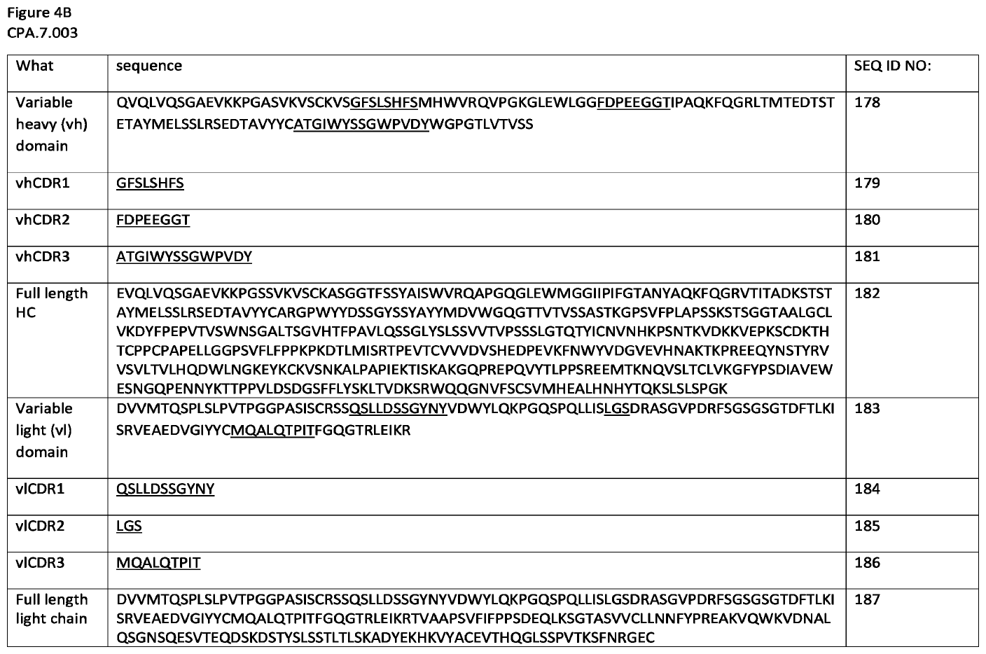

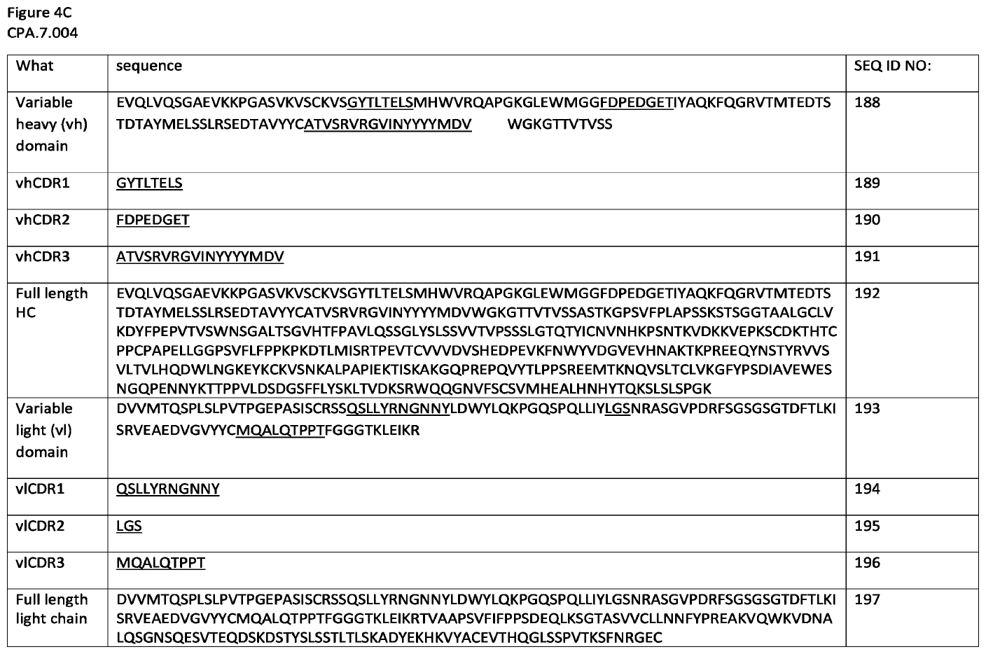

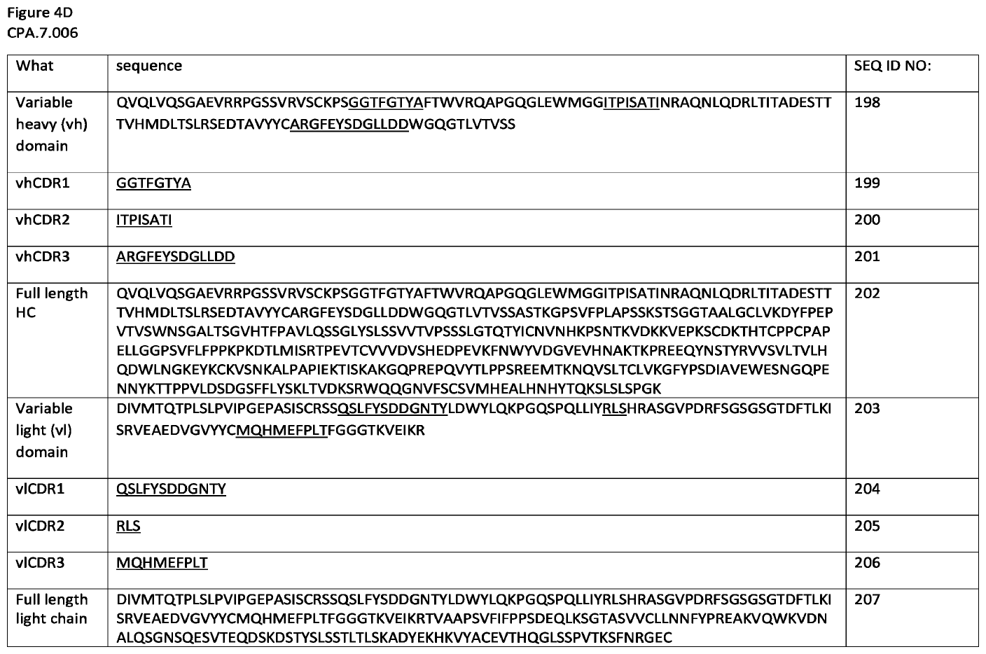

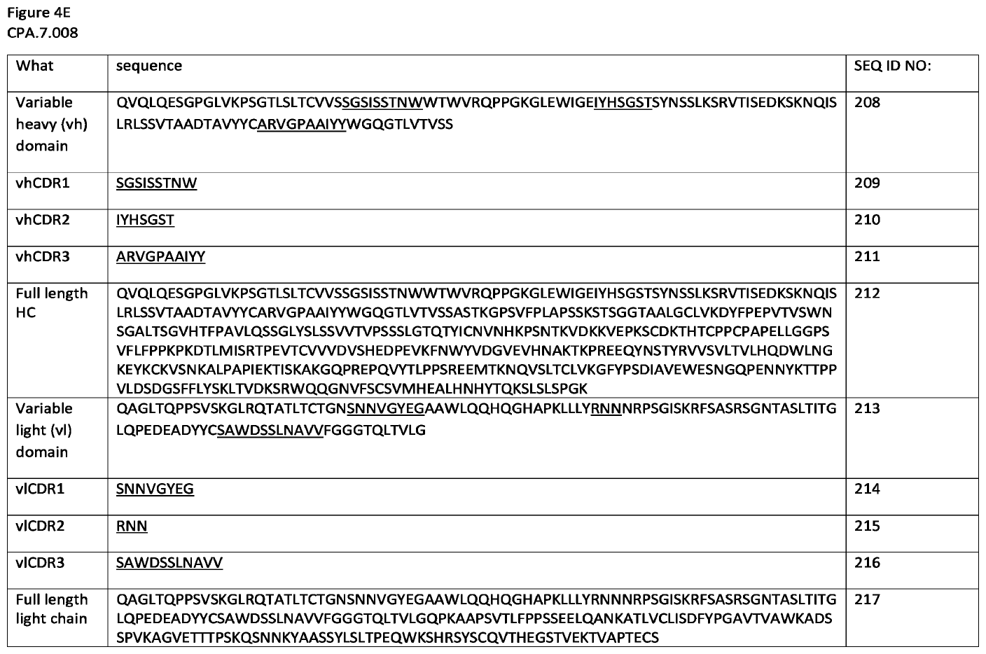

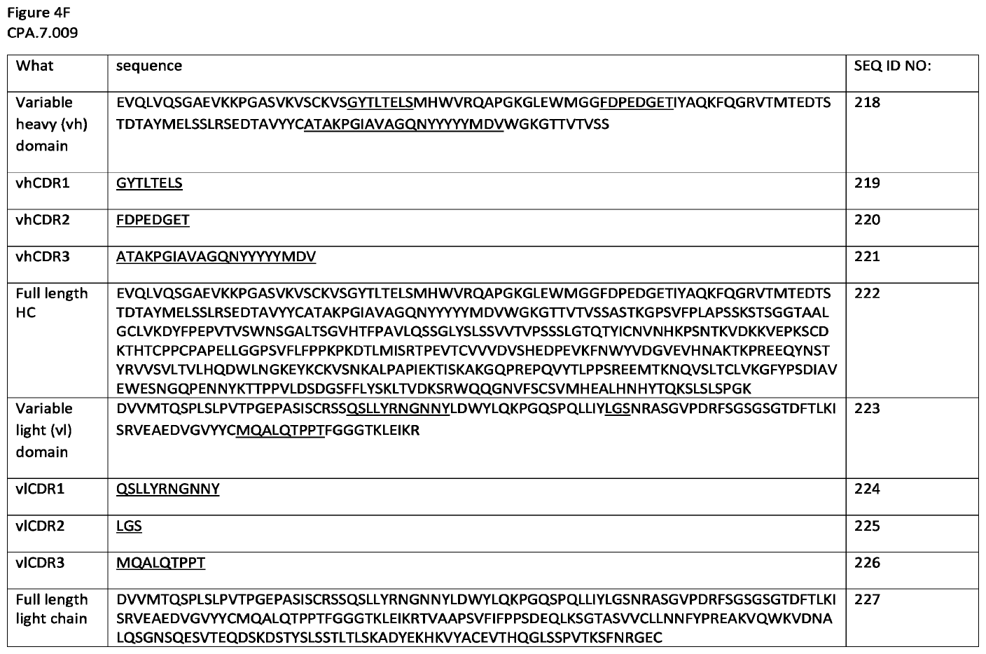

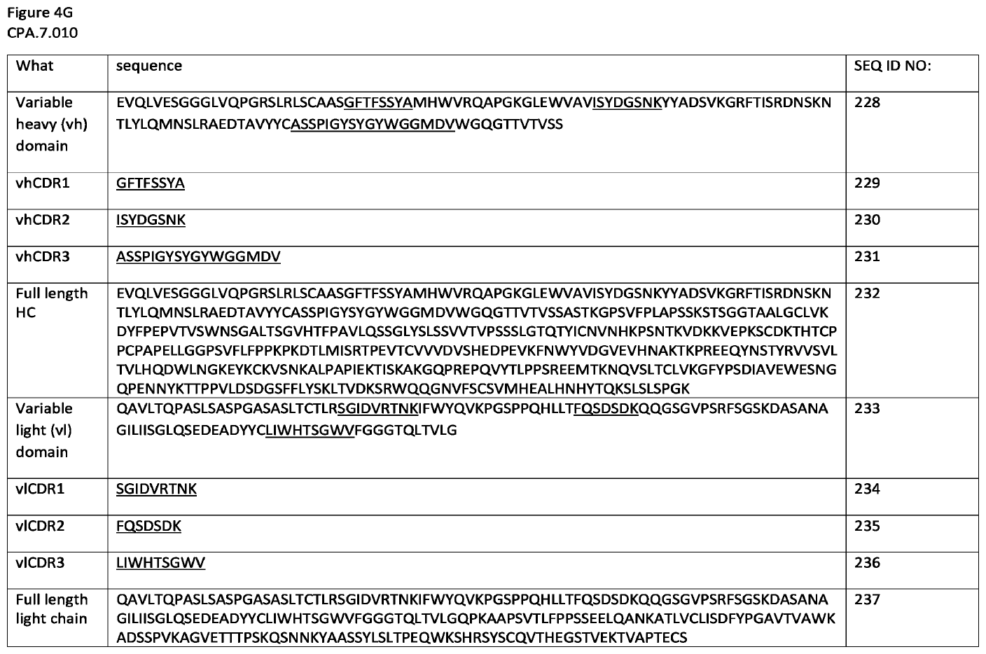

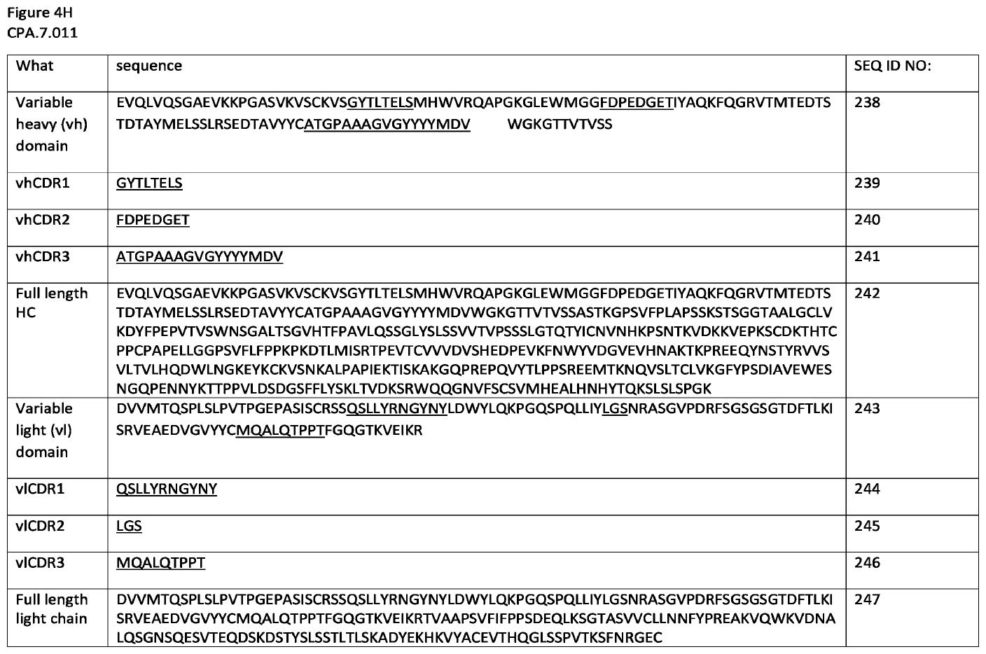

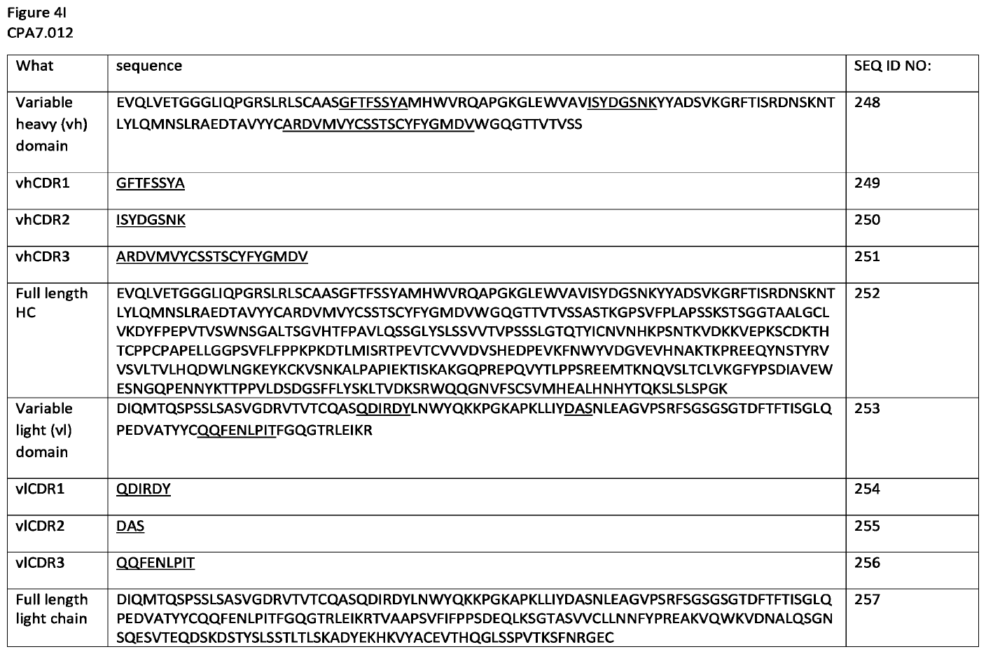

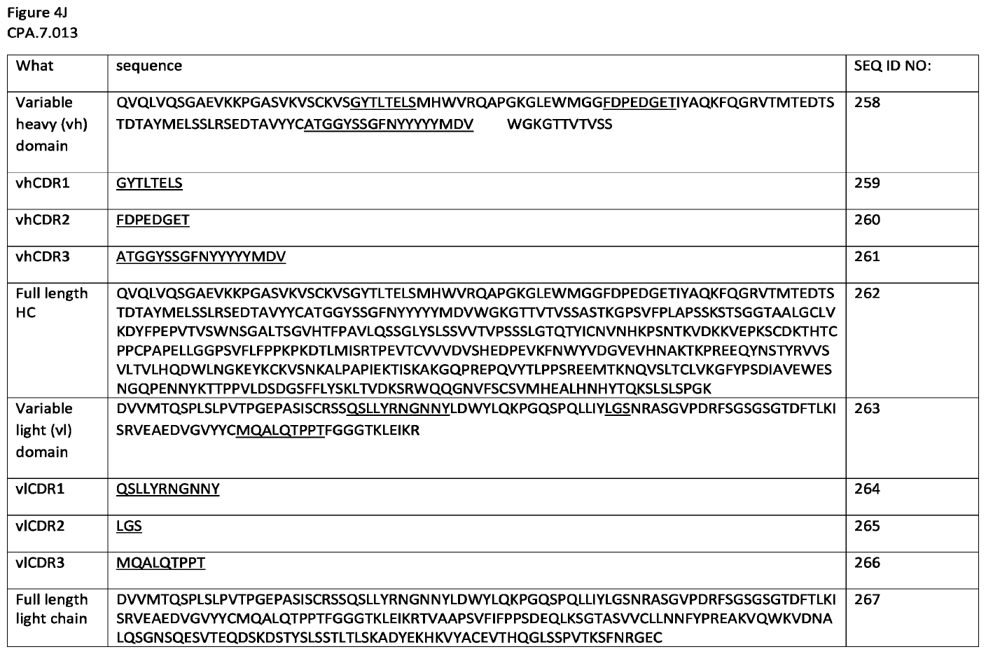

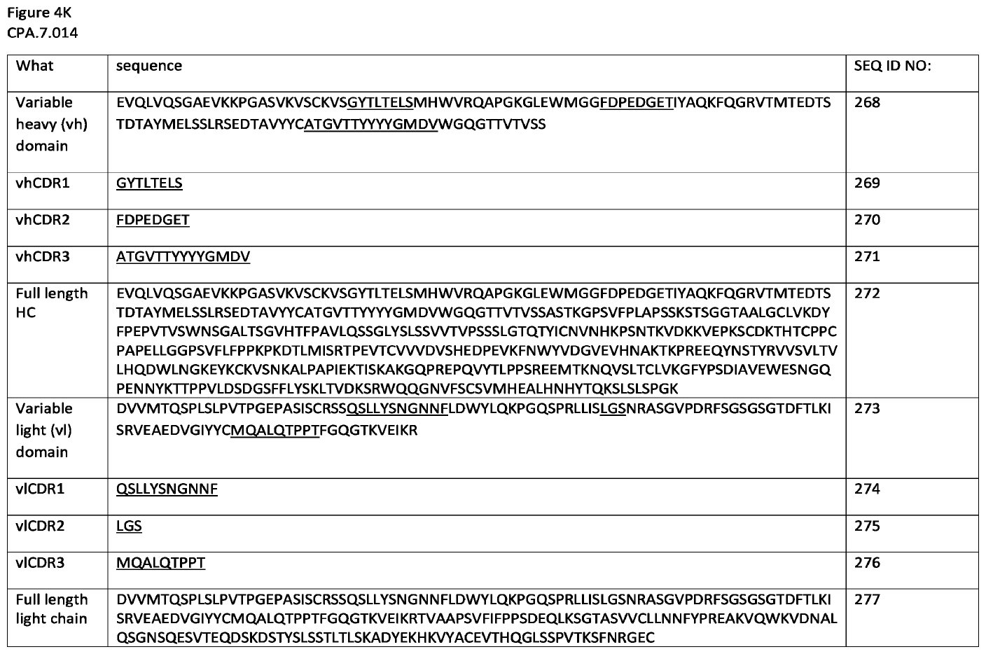

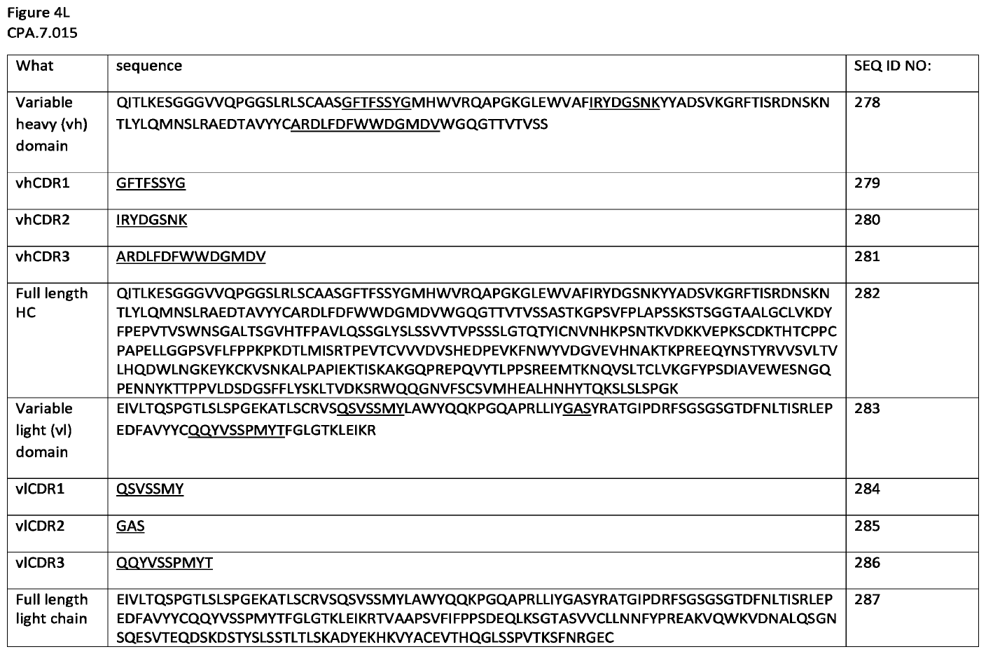

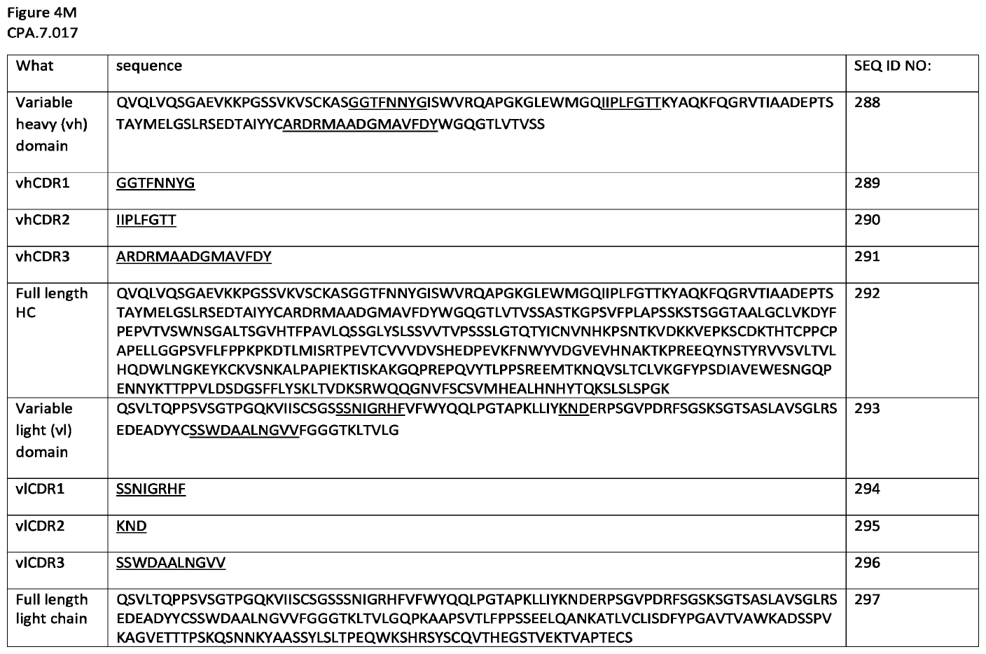

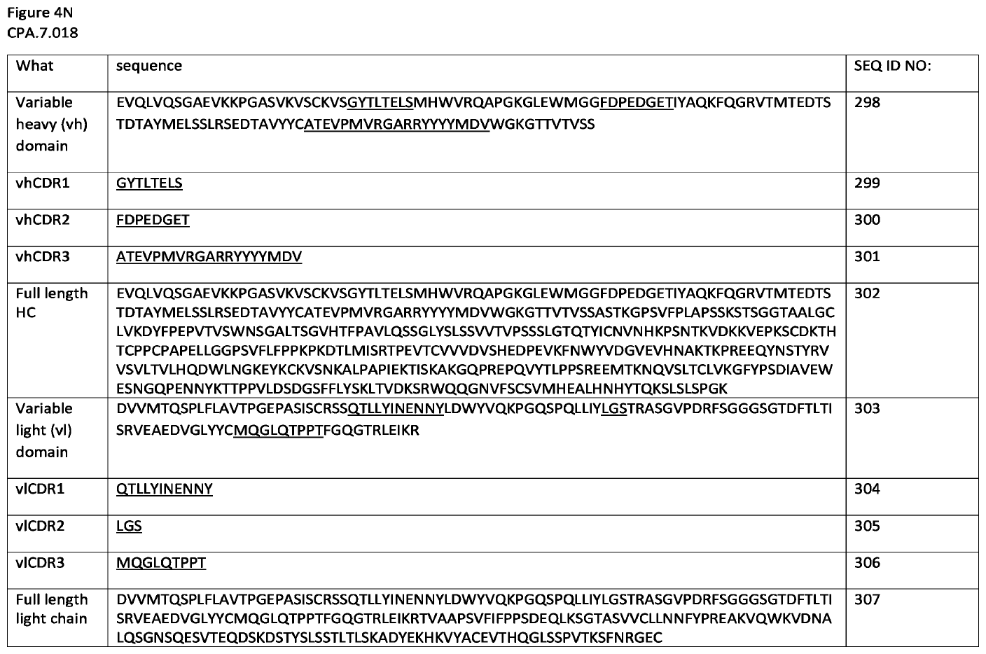

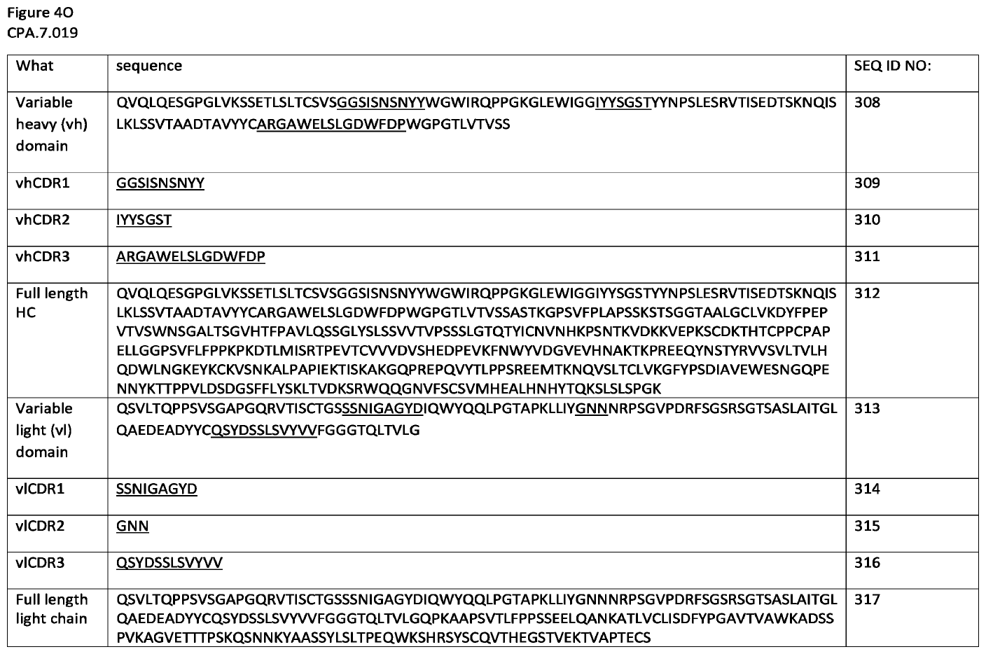

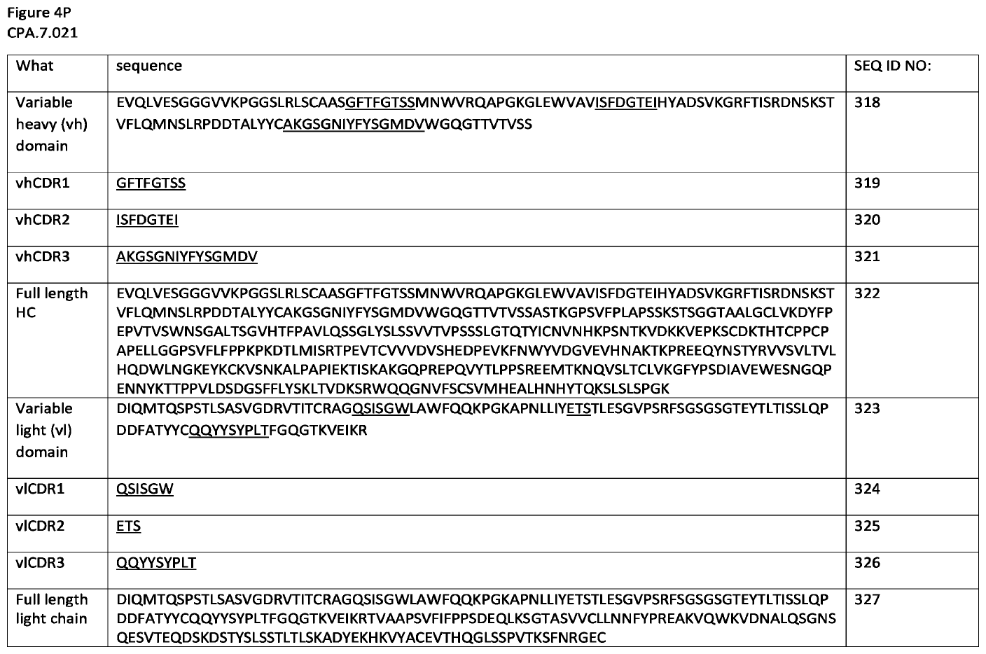

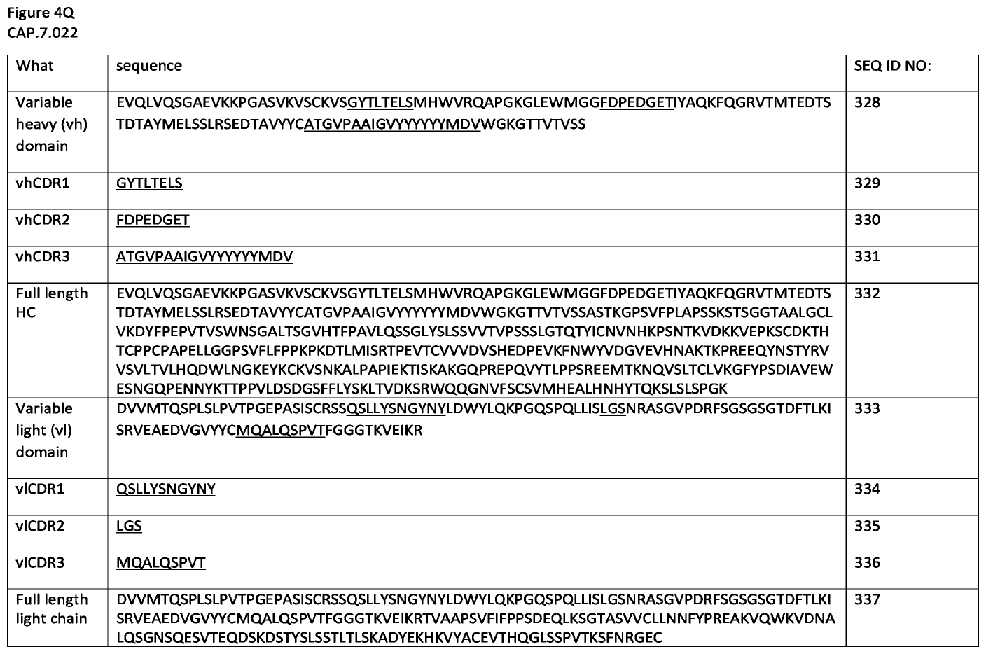

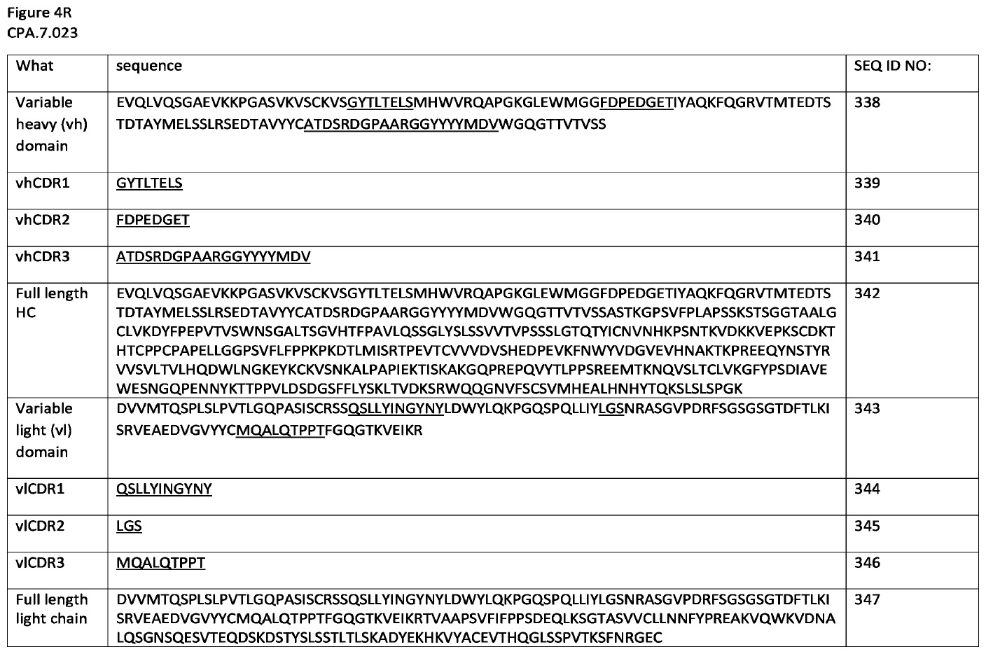

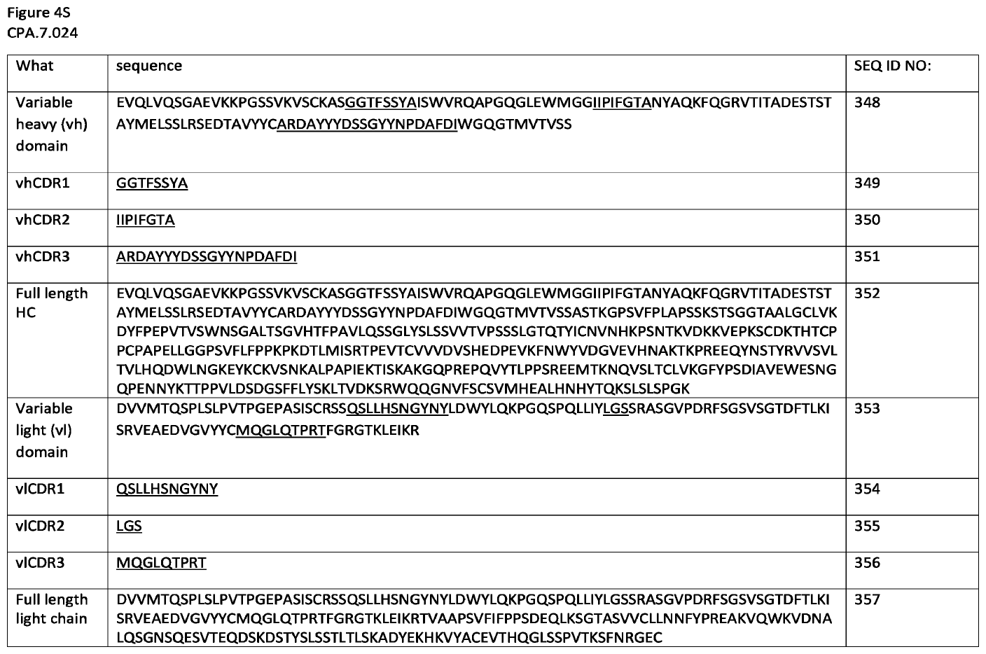

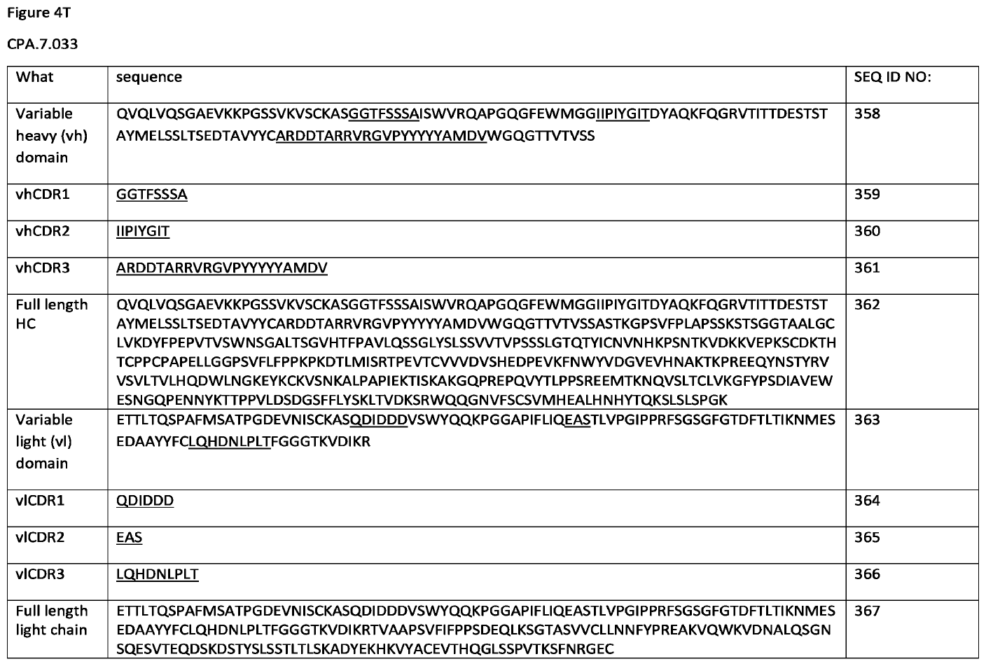

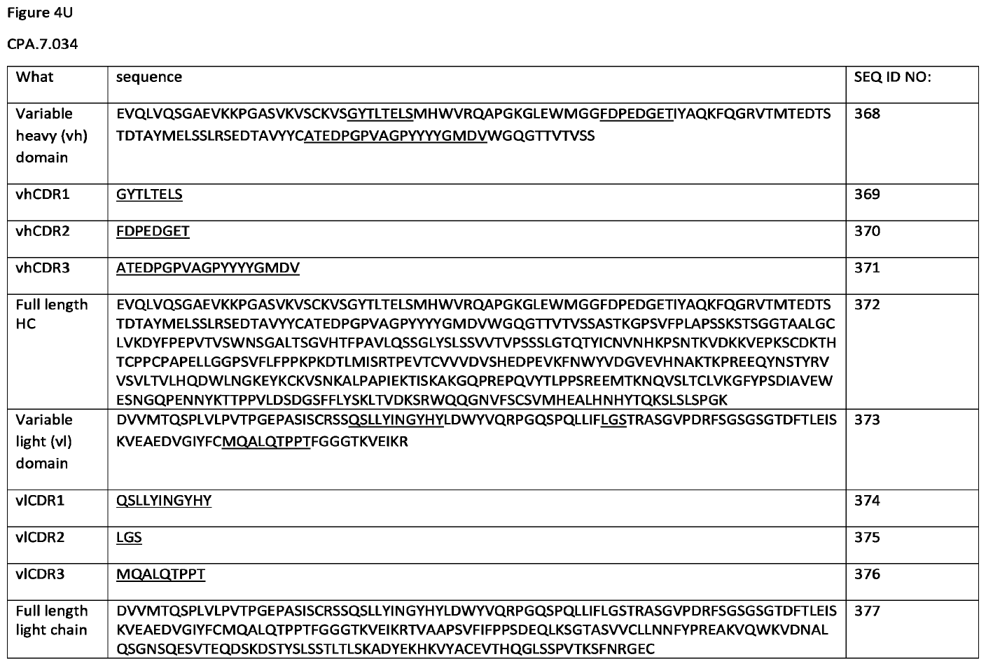

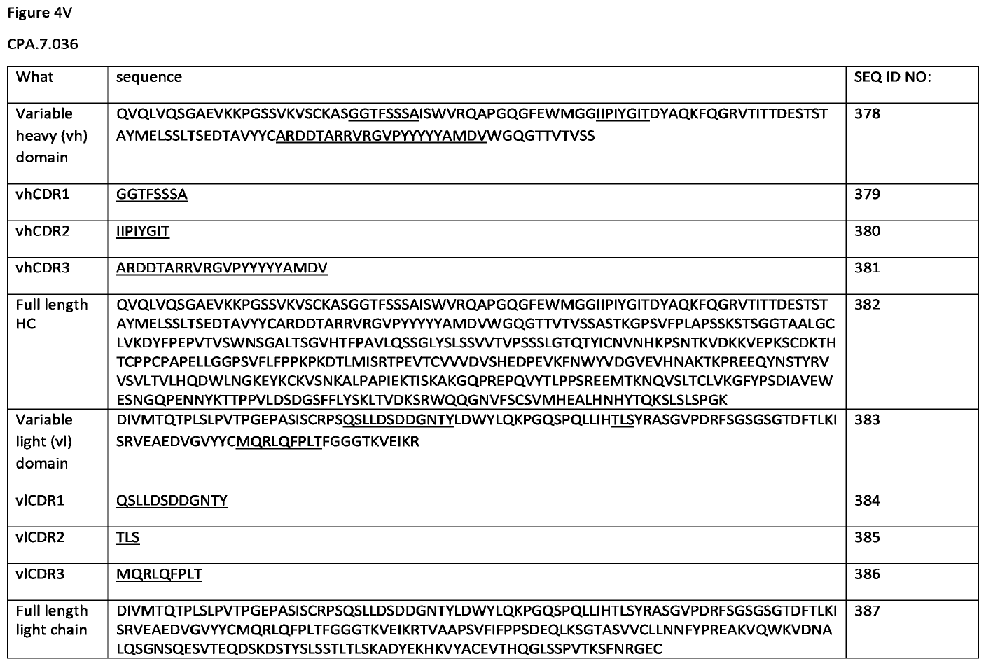

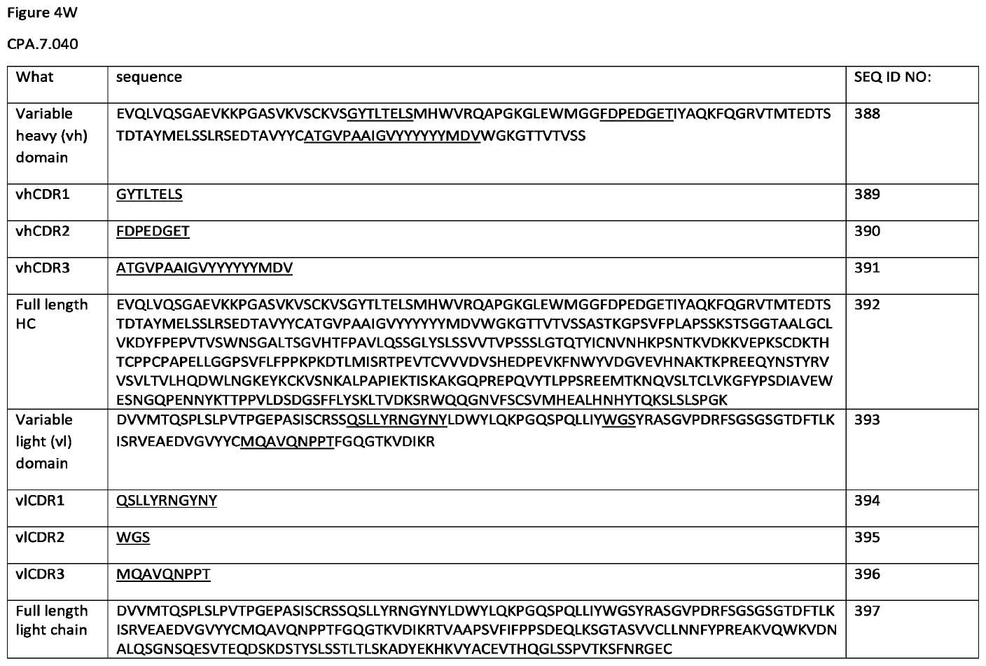

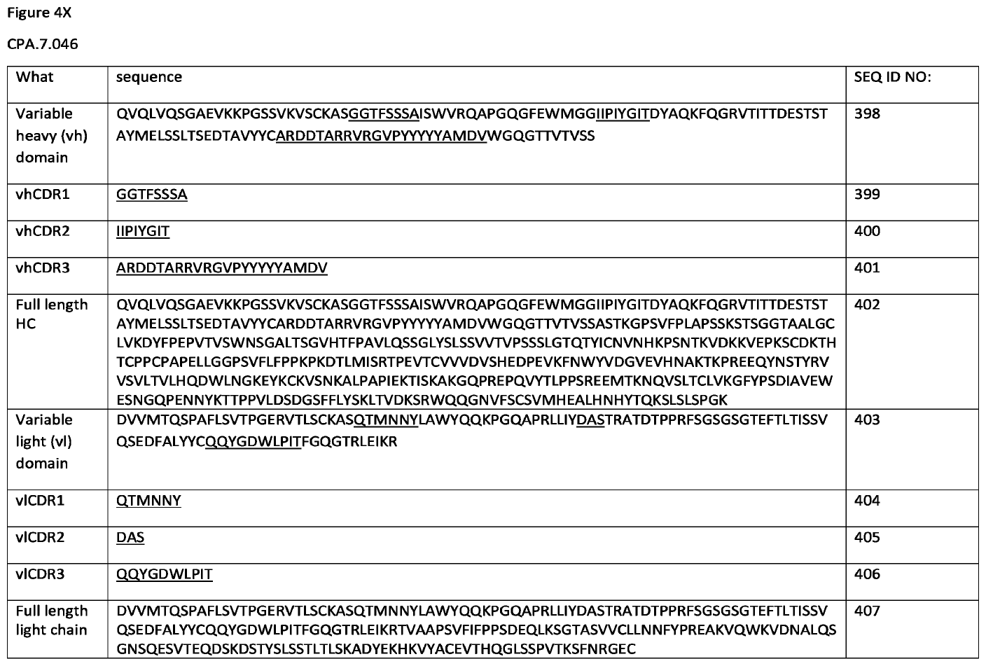

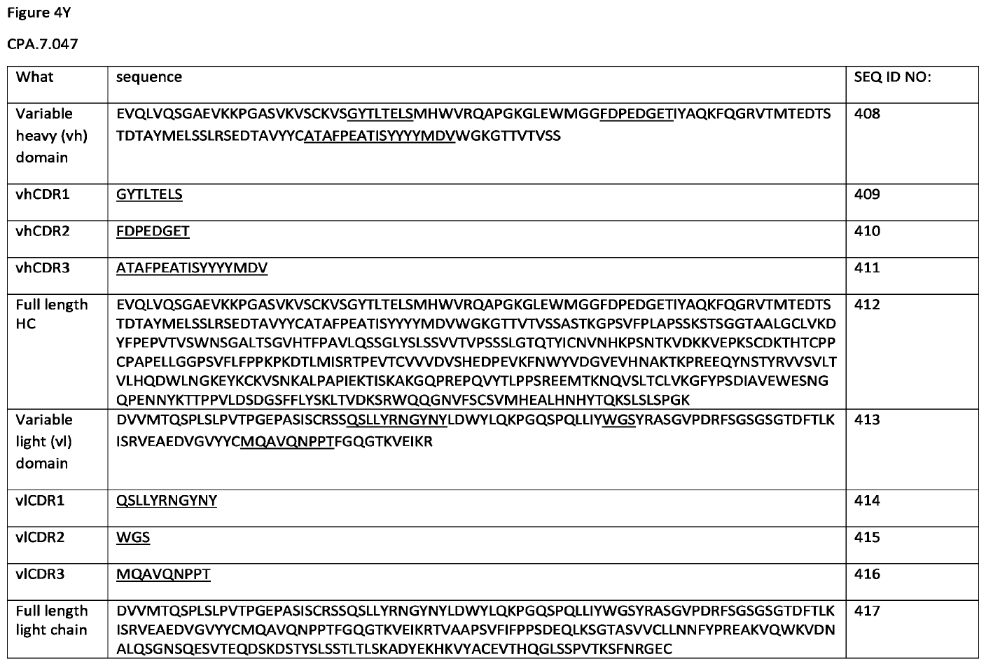

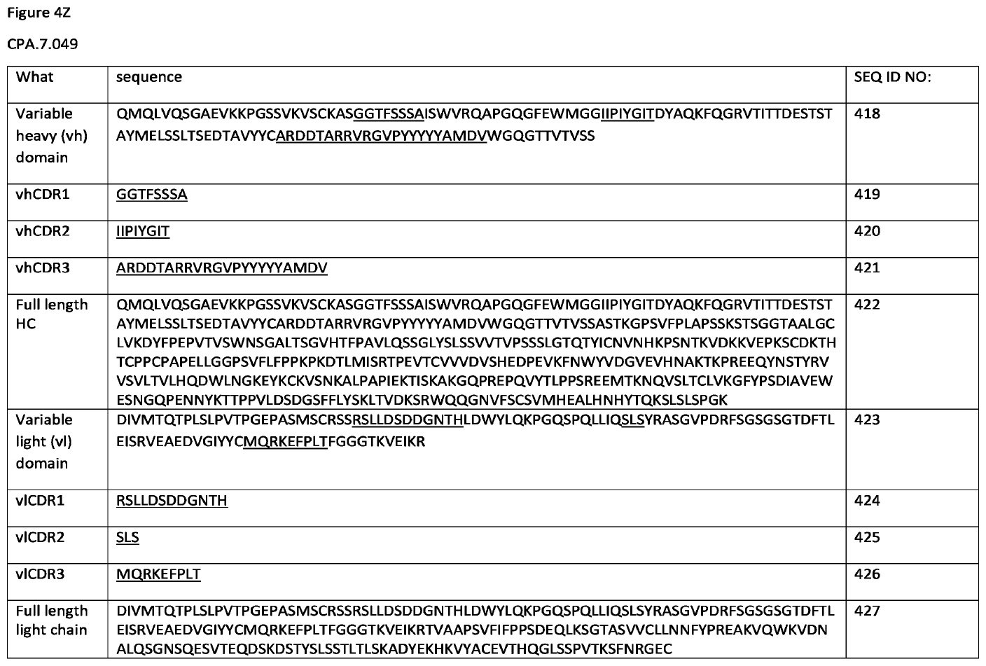

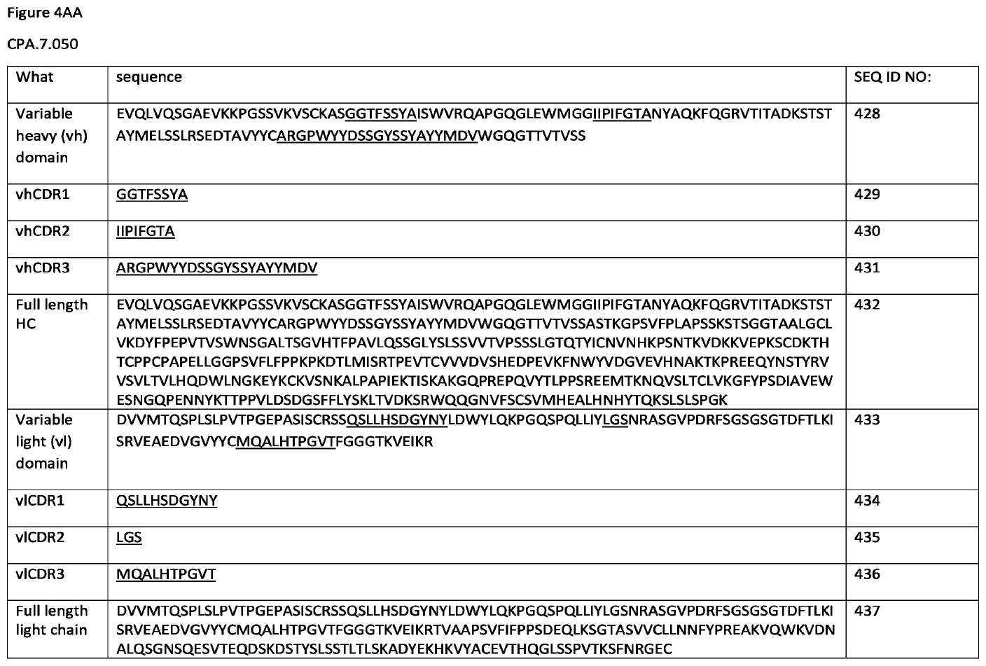

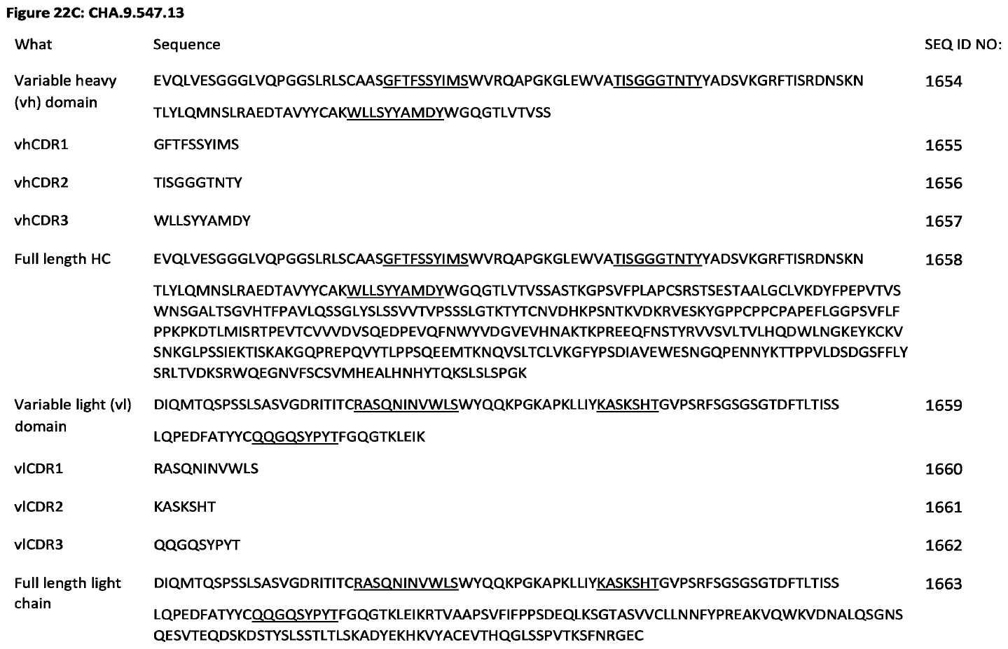

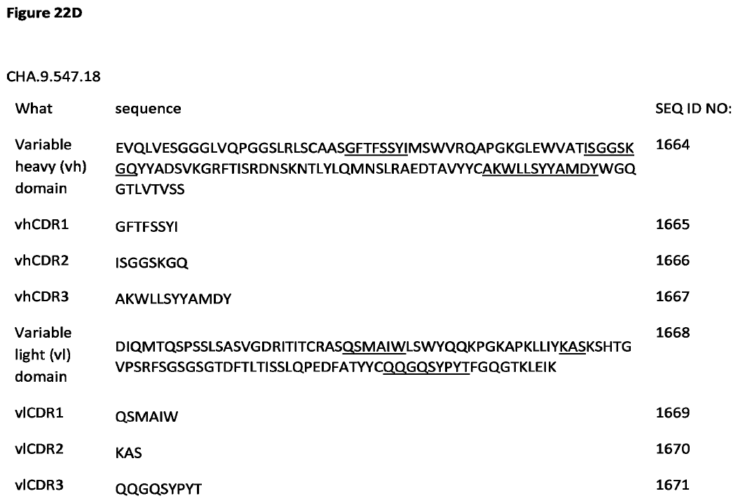

The sequence listing provides a number of sequences based on the Format of FIG. 22A-22D; reference is made to FIG. 4 of U.S. Ser. No. 62/513,916 (hereby expressly incorporated by reference) as a guide to the labeling of the sequences. The variable heavy domain is labeled with the identifier (e.g., “CPA.9.086”), with the next sequence following the format of FIG. 22A-22D of the present specification (identical to the format of FIG. 4, referenced above), in that the next sequence identifier is to the vhCDR1, the next to vhCDR2, with vhCDR3, the full length heavy chain, the variable light domain, vlCDR1, vlCDR2, vlCDR3 and the full length light chain. Thus an individual antibody has 10 associated sequence identifiers). Included in the sequence listing are the sequences of BM26 mouse IgG1 (BM26-M1) (WO2016/028656A1, Clone 3106) and BM29 mouse IgG1 (BM29-M1) (US2016/0176963A1, Clone 22G2). Unless noted, the full length HC sequences of the PVRIG and/or TIGIT antibodies are in the H4(S241P) format.

### D. PVRIG Proteins

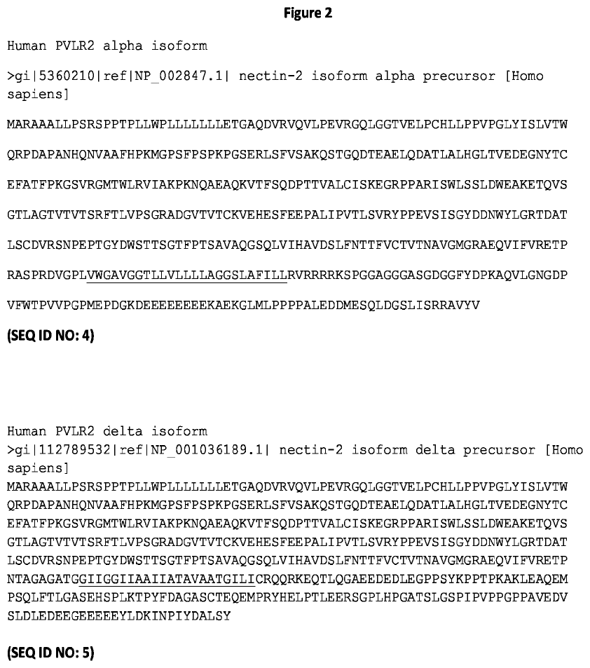

The present invention provides anti-PVRIG antibodies that specifically bind to PVRIG proteins and prevent activation by its ligand protein, PVRL2, a human plasma membrane glycoprotein. PVRIG, also called Poliovirus Receptor Related Immunoglobulin Domain Containing Protein, Q6DKI7 or C7orf15, relates to amino acid and nucleic acid sequences shown in RefSeq accession identifier NP_076975, shown in FIG. 1. The sequence of human Poliovirus receptor-related 2 protein (PVLR2, also known as nectin-2, CD112 or herpesvirus entry mediator B, (HVEB)), the binding partner of PVRIG (as shown in Example 5 of US Publication 2016/0244521), is shown in FIG. 2. The anti-PVRIG antibodies for use with the invention are specific for the PVRIG extracellular domain such that the binding of PVRIG and PVLR2 is blocked.

PVRIG is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). There are two methionines that can be start codons, but the mature proteins are identical.

Accordingly, as used herein, the term “PVRIG” or “PVRIG protein” or “PVRIG polypeptide” may optionally include any such protein, or variants, conjugates, or fragments thereof, including but not limited to known or wild type PVRIG, as described herein, as well as any naturally occurring splice variants, amino acid variants or isoforms, and in particular the ECD fragment of PVRIG.

As noted herein and more fully described below, anti-PVRIG antibodies that both bind to PVRIG and prevent activation by PVRL2 (e.g. most commonly by blocking the interaction of PVRIG and PVLR2), are used to enhance T cell and/or NK-cell activation and be used in treating diseases such as cancer and pathogen infection.

### E. TIGIT Proteins

The present invention provides anti-TIGIT antibodiesthat specifically bind to TIGIT proteins and prevent activation by its ligand protein, PVR, poliovirus receptor (aka CD155) a human plasma membrane glycoprotein. TIGIT, or T cell immunoreceptor with Ig and ITIM domains, is a co-inhibitory receptor protein also known as WUCAM, Vstm3 or Vsig9. TIGIT has an immunoglobulin variable domain, a transmembrane domain, and an immunoreceptor tyrosine-based inhibitory motif (ITIM) and contains signature sequence elements of the PVR protein family. The extracellular domain (ECD) sequences of TIGIT and of PVR are shown in FIG. 21. The antibodies for use with the invention are specific for the TIGIT ECD such that the binding of TIGIT and PVR is blocked

Accordingly, as used herein, the term “TIGIT” or “TIGIT protein” or “TIGIT polypeptide” may optionally include any such protein, or variants, conjugates, or fragments thereof, including but not limited to known or wild type TIGIT, as described herein, as well as any naturally occurring splice variants, amino acid variants or isoforms, and in particular the ECD fragment of TIGIT.

As noted herein and more fully described below, anti-TIGIT antibodies (including antigen-binding fragments) that both bind to TIGIT and prevent activation by PVR (e.g., most commonly by blocking the interaction of TIGIT and PVR), are used to enhance T cell and/or NK cell activation and be used in treating diseases such as cancer and pathogen infection.

### V. ANTIBODIES

As is discussed below, the term “antibody” is used generally. Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to monoclonal antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M). The sequences depicted herein use the 356D/358M allotype, however the other allotype is included herein. That is, any sequence inclusive of an IgG1 Fc domain included herein can have 356E/358L replacing the 356D/358M allotype.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vhCDRs (e.g. vlCDR1, vlCDR2 and vlCDR3). A useful comparison of CDR numbering is as below, see Lafranc et al., Dev. Comp. Immunol. 27(1):55-77 (2003):

| | Kabat + | | | | | |
|---|---|---|---|---|---|---|
| | Clothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 53-55 | 47-58 |
| vhCDR3 | 95-102 | 105-117 | 95-102 | 95-102 | 96-101 | 93-101 |
| vlCDR1 | 24-34 | 27-38 | 24-34 | 24-34 | 26-32 | 30-36 |
| vlCDR2 | 50-56 | 56-65 | 50-56 | 50-56 | 50-52 | 46-55 |
| vlCDR3 | 89-97 | 105-117 | 89-97 | 89-97 | 91-96 | 89-96 |

Throughout the present specification, the Kabat numbering system is generally used when referring to a residue in the variable domain (approximately, residues 1-107 of the light chain variable region and residues 1-113 of the heavy chain variable region) and the hinge and the EU numbering system for Fc regions (e.g, Kabat et al., supra (1991)).

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used, or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat.

The light chain generally comprises two domains, the variable light domain (containing the light chain CDRs and together with the variable heavy domains forming the Fv region), and a constant light chain region (often referred to as CL or Cκ). In general, either the constant lambda or constant kappa domain can be used, with lambda generally finding use in the invention.

Another region of interest for additional substitutions, outlined below, is the Fc region.

### A. Chimeric and Humanized Antibodies

In some embodiments, the anti-PVRIG antibodies and/or anti-TIGIT antibodies herein can be derived from a mixture from different species, e.g. a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, all entirely incorporated by reference). The humanized antibody optimally also will comprise at least a portion, and usually all, of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference.

Thus, the vhCDRs and vhCDRs from any of the enumerated antibodies herein may be humanized (or “rehumanized”, for those that were already humanized).

In certain embodiments, the antibodies for use with the invention comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene excluding the CDRs. That is, the CDRs may be murine, but the framework regions of the variable region (either heavy or light) can be at least 96%, 97%, 98%, or 99% identical in amino acid sequence to the framework amino acids encoded by one human germline immunoglobulin gene.

Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene (again, prior to the introduction of any variants herein; that is, the number of variants is generally low).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### B. Optional Antibody Engineering

The anti-PVRIG and/or anti-TIGIT antibodies for use with the invention can be modified, or engineered, to alter the amino acid sequences by amino acid substitutions. As discussed herein, amino acid substitutions can be made to alter the affinity of the CDRs for the protein (e.g., TIGIT or PVRIG, including both increasing and decreasing binding), as well as to alter additional functional properties of the antibodies. For example, the antibodies may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of Cm is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In still another embodiment, the anti-PVRIG antibodies and/or anti-TIGIT antibodies can be modified to abrogate in vivo Fab arm exchange, in particular when IgG4 constant domains are used. Specifically, this process involves the exchange of IgG4 half-molecules (one heavy chain plus one light chain) between other IgG4 antibodies that effectively results in antibodies which are functionally monovalent. Mutations to the hinge region and constant domains of the heavy chain can abrogate this exchange (see Aalberse, R C, Schuurman J., 2002, Immunology 105:9-19). As outlined herein, a mutation that finds particular use in the present invention is the S241P in the context of an IgG4 constant domain. IgG4 finds use in the present invention as it has no significant effector function, and is thus used to block the receptor binding to its ligand without cell depletion (e.g. PVRIG to PVRL2 or TIGIT to PVR).

In some embodiments, amino acid substitutions can be made in the Fc region, in general for altering binding to FcγR receptors. By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII-1 (CD16), and FcγRIII-2 (CD16-2), as well as any undiscovered mouse FcγRs or FcγR isoforms or allotypes.

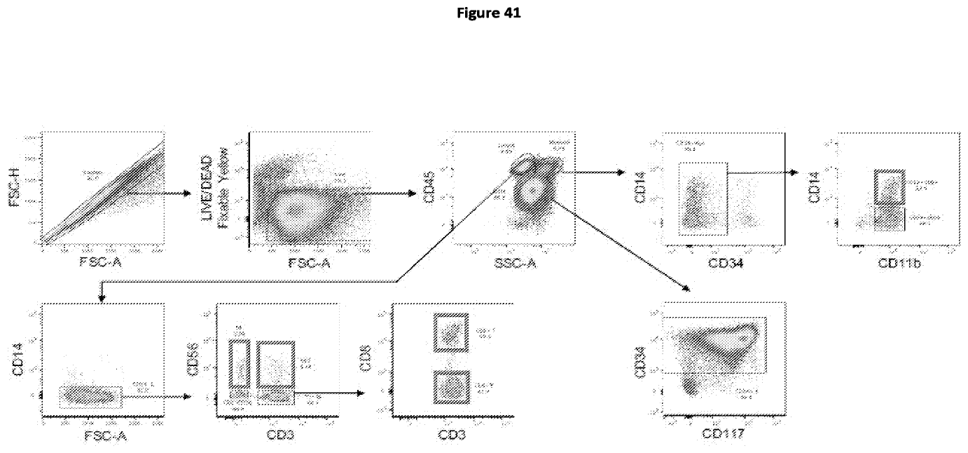

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell. Similarly, decreased binding to FcγRIIb (an inhibitory receptor) can be beneficial as well in some circumstances. Amino acid substitutions that find use in the present invention include those listed in U.S. Ser. No. 11/124,620 (particularly FIG. 41) and U.S. Pat. No. 6,737,056, both of which are expressly incorporated herein by reference in their entirety and specifically for the variants disclosed therein.

In yet another example, the Fc region is modified to increase the ability of the anti-PVRIG antibodies and/or anti-TIGIT antibodies to mediate antibody dependent cellular cytotoxicity (ADCC) and/or to increase the affinity of the antibody for an Fcγ receptor, and/or increase FcRn binding, by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (see Chan C A and Carter P J (2010) Nature Rev Immunol 10:301-316).

In addition, the anti-PVRIG antibodies and/or anti-TIGIT antibodies for use with the invention are modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the Cm or CL region to contain a salvage receptor binding epitope taken from two loops of a CH2 domain of an Fc region of an IgG, as described in U.S. Pat. Nos. 5,869,046 and 6,121,022 by Presta et al. Additional mutations to increase serum half-life are disclosed in U.S. Pat. Nos. 8,883,973, 6,737,056 and 7,371,826 and include 428L, 434A, 434S, and 428L/434S.

In still another embodiment, the glycosylation of anti-PVRIG antibodies and/or anti-TIGIT antibodies can be modified. For example, an aglycosylated antibody can be made (e.g., the antibody lacks glycosylation). Glycosylation can be altered to, for example, increase the affinity of the antibody for antigen or reduce effector function such as ADCC. Such carbohydrate modifications can be accomplished by, for example, altering one or more sites of glycosylation within the antibody sequence, for example N297. For example, one or more amino acid substitutions can be made that result in elimination of one or more variable region framework glycosylation sites to thereby eliminate glycosylation at that site, with an alanine replacement finding use in some embodiments.

Additionally or alternatively, an anti-PVRIG antibody and/or anti-TIGIT antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. See for example, U.S. Patent Publication No. 20040110704 and WO 2003/035835.

Another modification of the anti-PVRIG antibodies and/or anti-TIGIT antibodies herein that is contemplated by the invention is PEGylation or the addition of other water soluble moieties, typically polymers, e.g., in order to enhance half-life. An antibody can be PEGylated to, for example, increase the biological (e.g., serum) half-life of the antibody as is known in the art.

In addition to substitutions made to alter binding affinity to FcγRs and/or FcRn and/or increase in vivo serum half-life, additional antibody modifications can be made, as described in further detail below.

In some cases, affinity maturation is done. Amino acid modifications in the CDRs are sometimes referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, it may be desirable to decrease the affinity of an antibody to its antigen.

In some embodiments, one or more amino acid modifications are made in one or more of the CDRs of the anti-PVRIG antibodies and/or anti-TIGIT antibodies for use with the invention (for example, to the PVRIG CDRs or the TIGIT CDRs). In general, only 1 or 2 or 3-amino acids are substituted in any single CDR, and generally no more than from 1, 2, 3. 4, 5, 6, 7, 8, 9, or 10 changes are made within a set of 6 CDRs (e.g., vhCDR1-3 and vlCDR1-3). However, it should be appreciated that any combination of no substitutions, 1, 2 or 3 substitutions in any CDR can be independently and optionally combined with any other substitution.

Affinity maturation can be done to increase the binding affinity of the antibody for the antigen by at least about 10% to 50-100-150% or more, or from 1 to 5 fold as compared to the “parent” antibody. In some embodiments, affinity matured antibodies will have nanomolar or even picomolar affinities for the antigen. Affinity matured antibodies are produced by known procedures. The correlation of affinity and efficacy is discussed below.

Alternatively, amino acid modifications can be made in one or more of the CDRs of the antibodies for use with the invention that are “silent”, e.g., that do not significantly alter the affinity of the antibody for the antigen. These can be made for a number of reasons, including optimizing expression (as can be done for the nucleic acids encoding the antibodies for use with the invention).

Thus, included within the definition of the CDRs and anti-PVRIG antibodies and/or anti-TIGIT antibodies for use with the invention are variant CDRs and anti-PVRIG antibodies and/or anti-TIGIT antibodies; that is, the anti-PVRIG antibodies and/or anti-TIGIT antibodies for use with the invention can include amino acid modifications in one or more of the CDRs of the enumerated antibodies for use with the invention. In addition, as outlined below, amino acid modifications can also independently and optionally be made in any region outside the CDRs, including framework and constant regions.

### VI. ANTI-PVRIG ANTIBODIES

Specific binding for PVRIG or a PVRIG epitope can be exhibited, for example, by an antibody having a KD of at least about 10⁻⁴M, at least about 10⁻⁵ M, at least about 10⁻⁶ M, at least about 10⁻⁷ M, at least about 10⁻⁸M, at least about 10⁻⁹M, alternatively at least about 10⁻¹⁰ M, at least about 10⁻¹¹ M, at least about 10⁻¹²M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the PVRIG antigen or epitope.

Generally, for optimal binding to PVRIG expressed on the surface of NK and T-cells, the antibodies preferably have a KD less 50 nM and most preferably less than 1 nM, with less than 0.1 nM and less than 1 pM and 0.1 pM finding use in the methods of the invention.

Also, specific binding for a particular antigen or an epitope can be exhibited, for example, by an antibody having a KA or Ka for a PVRIG antigen or epitope of at least 20-, 50-, 100-, 500-, 1000-, 5,000-, 10,000- or more times greater for the epitope relative to a control, where KA or Ka refers to an association rate of a particular antibody-antigen interaction.

In some embodiments, the anti-PVRIG antibodies for use with the invention bind to human PVRIG with a K_{D} of 100 nM or less, 50 nM or less, 10 nM or less, or 1 nM or less (that is, higher binding affinity), or 1 pM or less, wherein K_{D} is determined by known methods, e.g. surface plasmon resonance (SPR, e.g. Biacore assays), ELISA, KINEXA, and most typically SPR at 25° or 37° C.

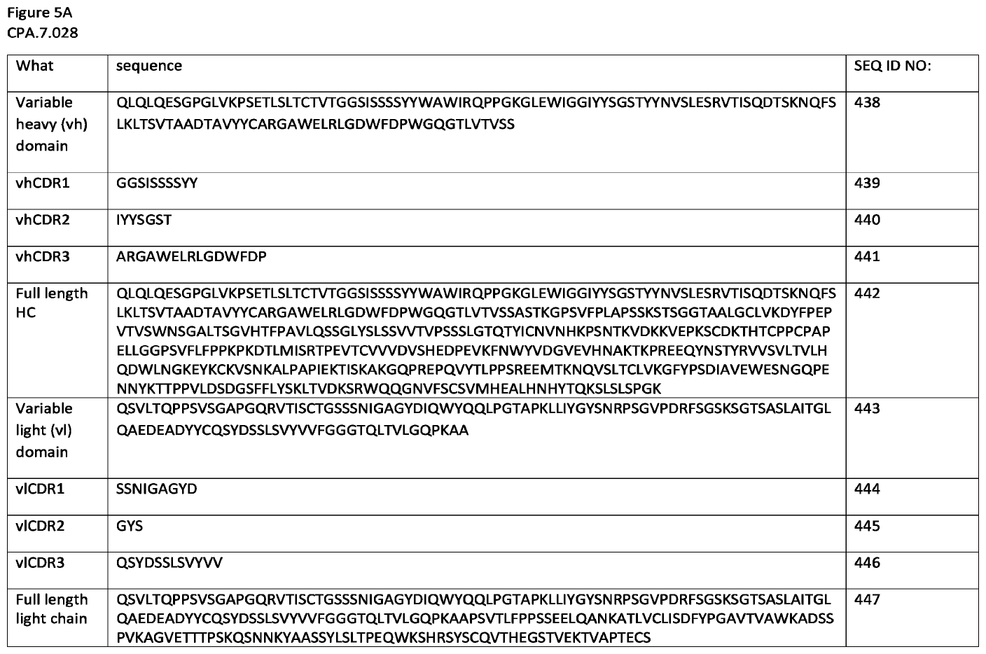

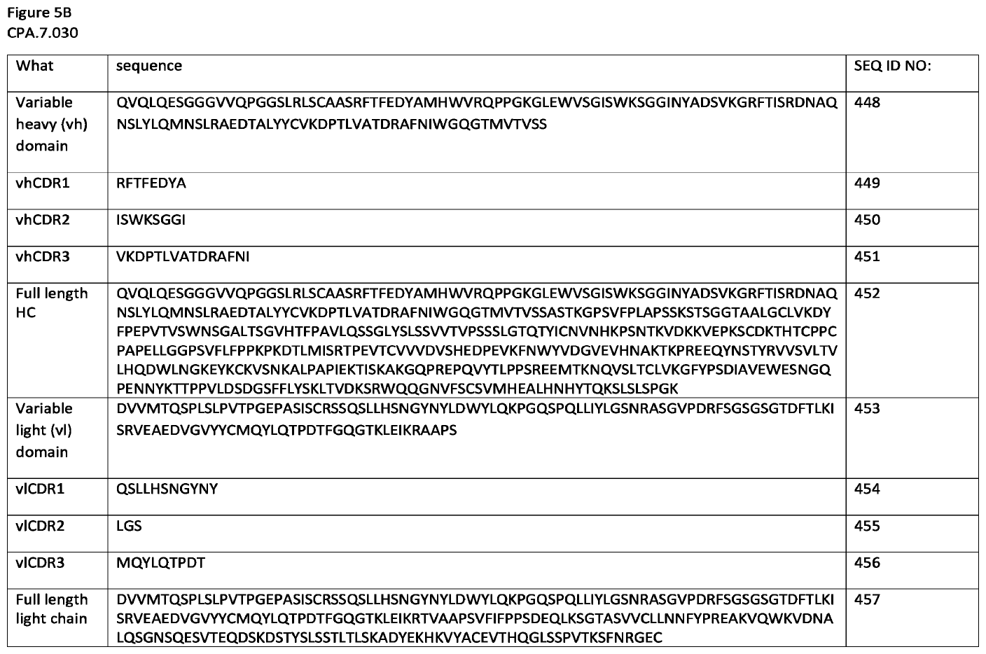

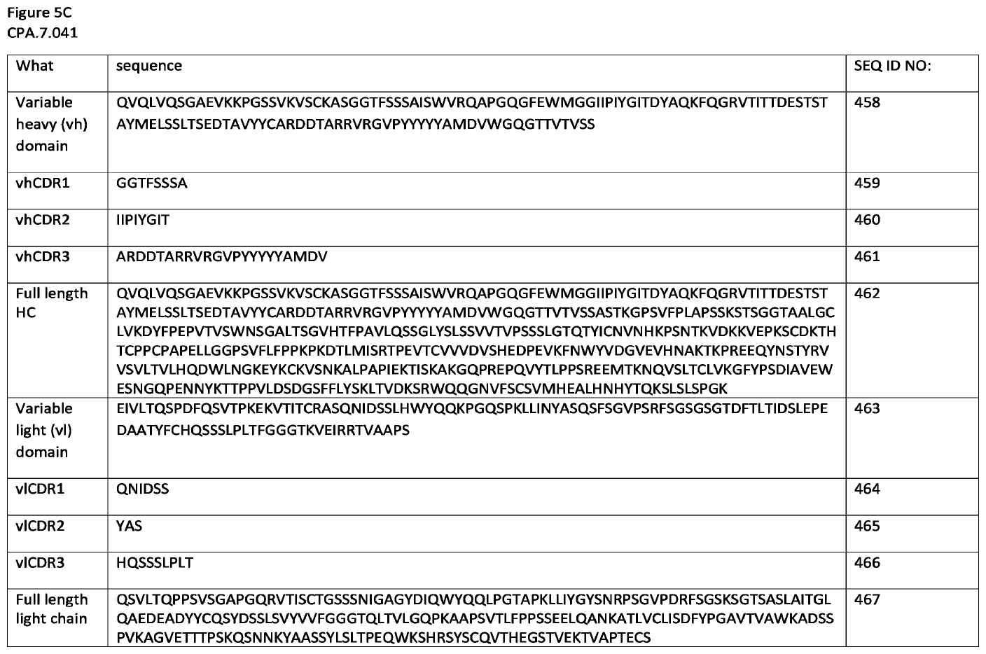

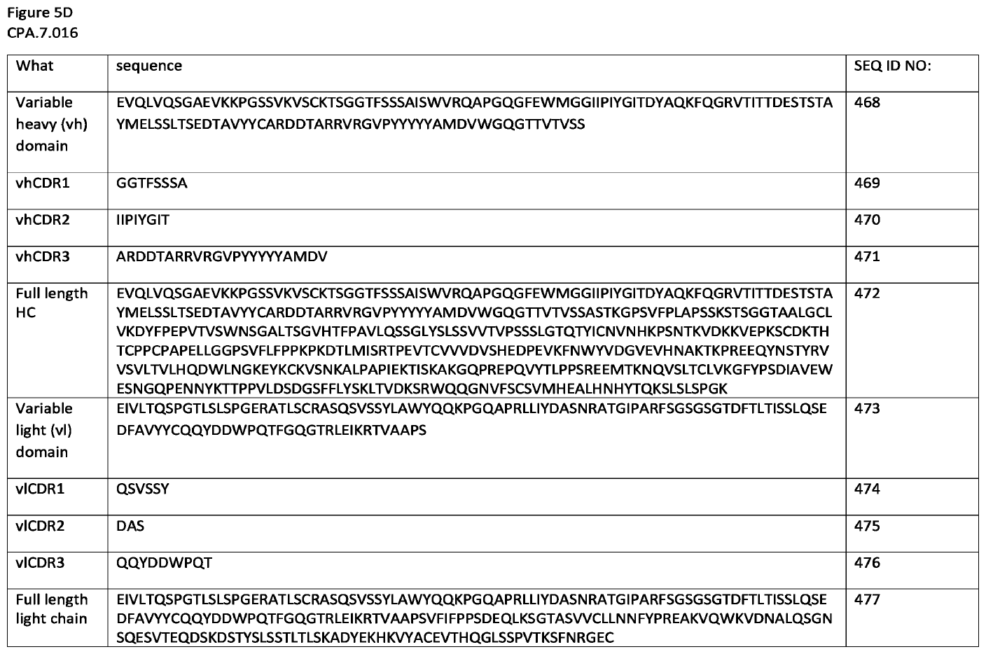

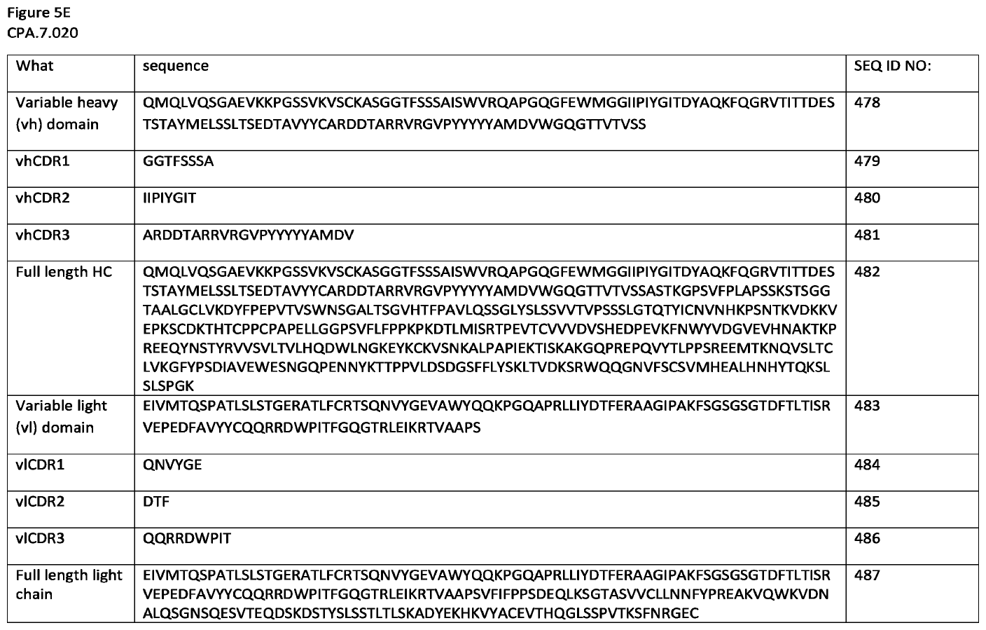

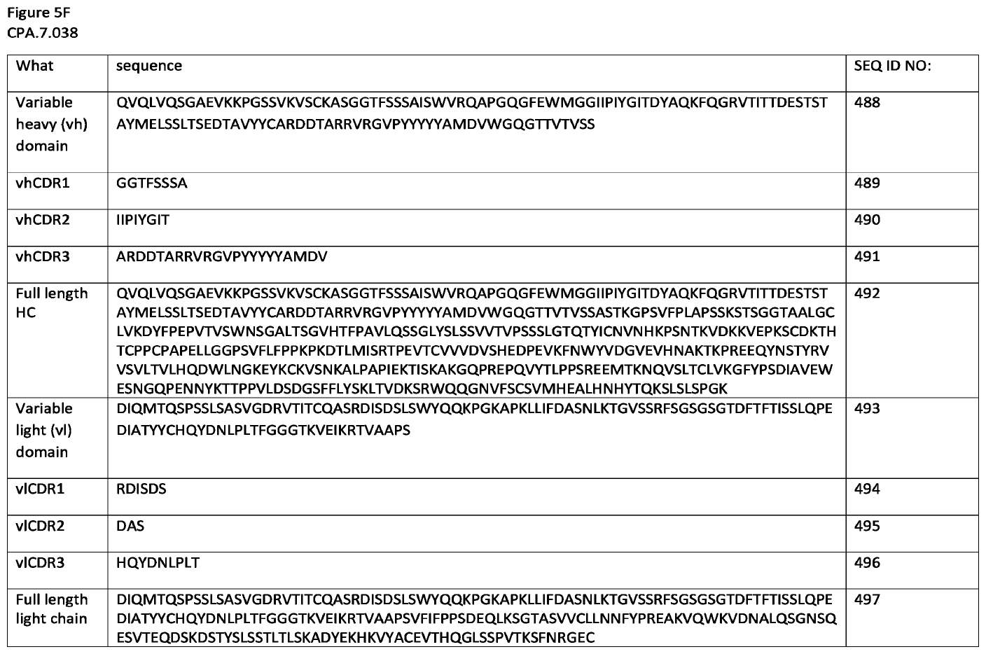

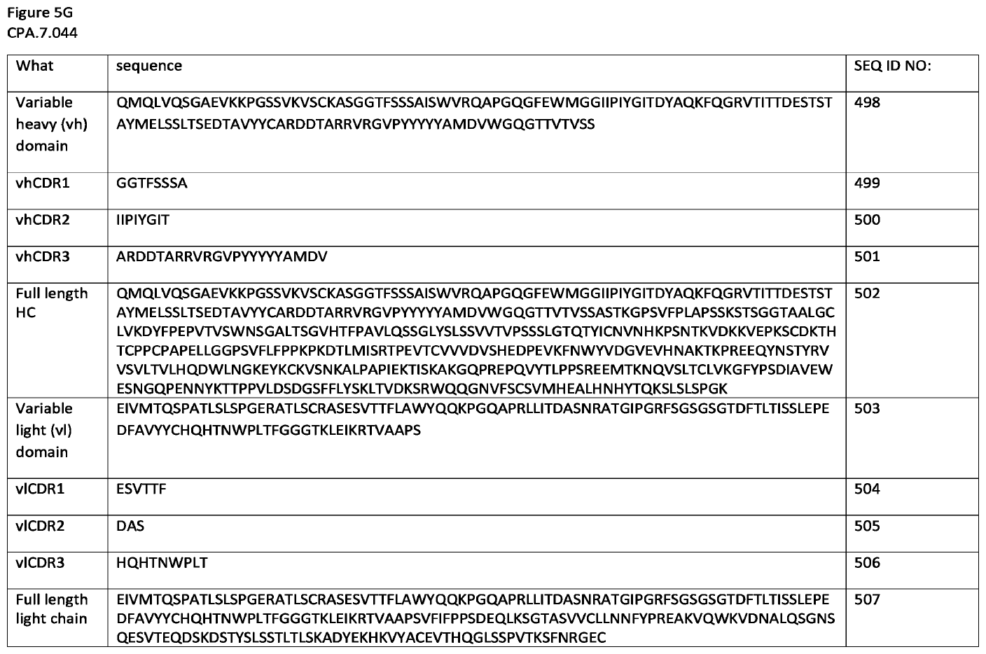

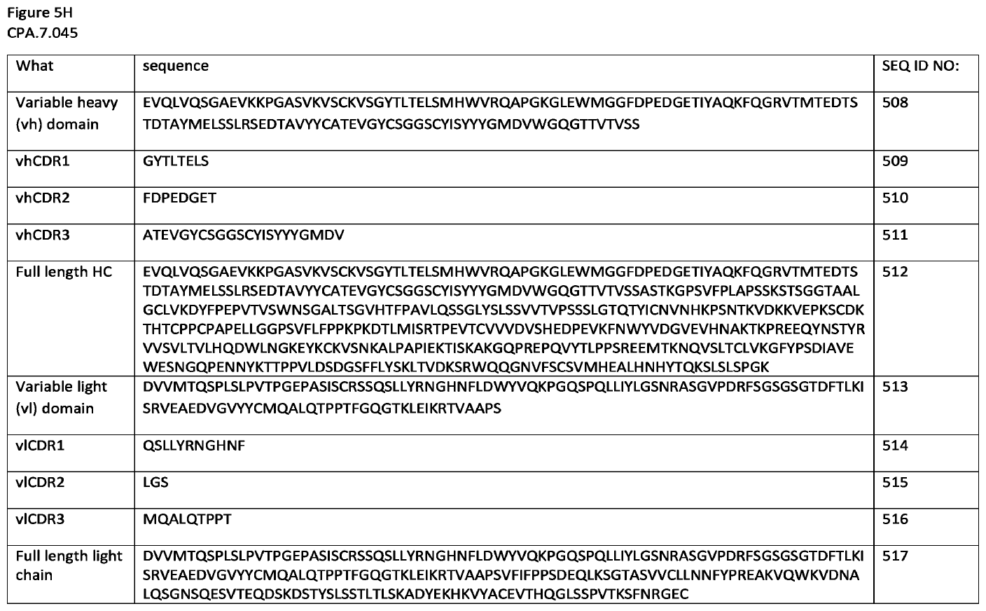

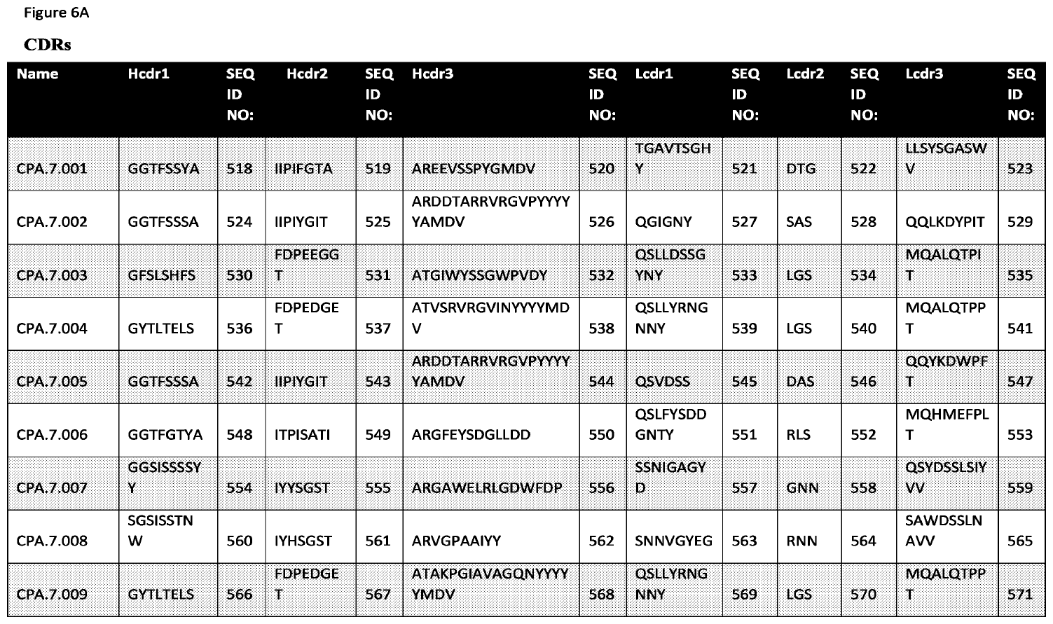

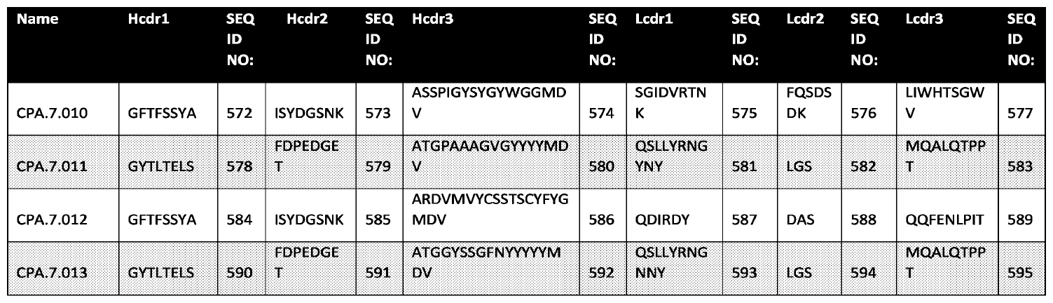

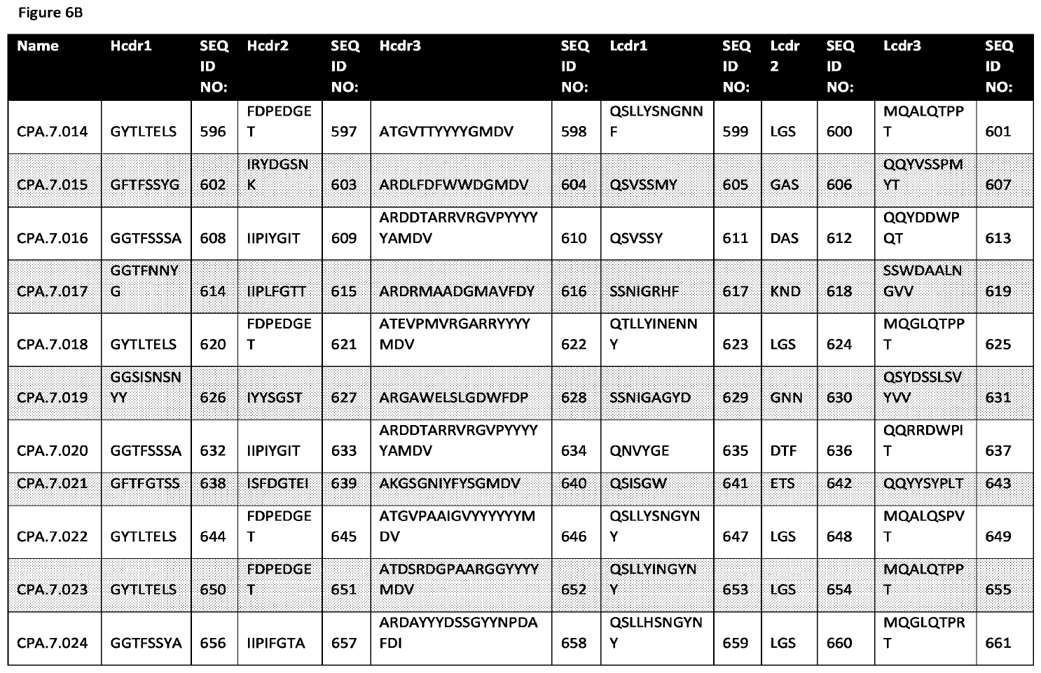

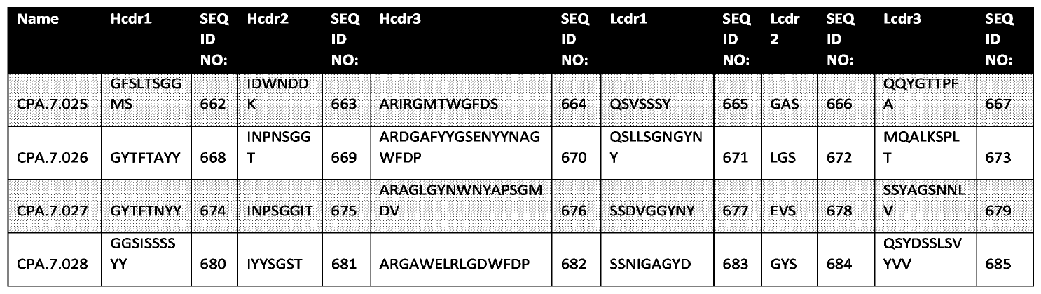

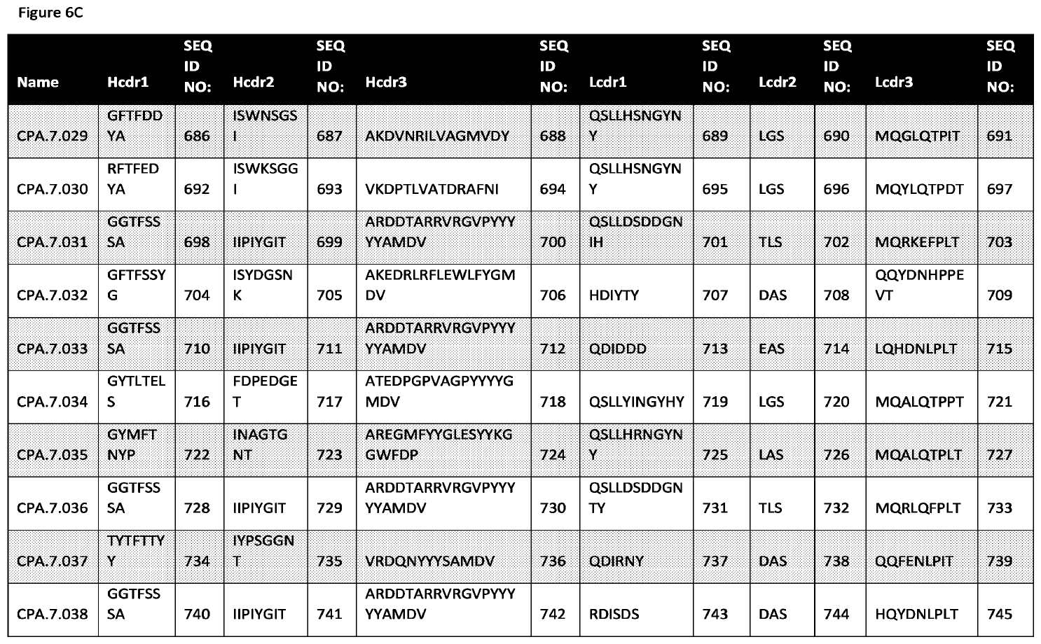

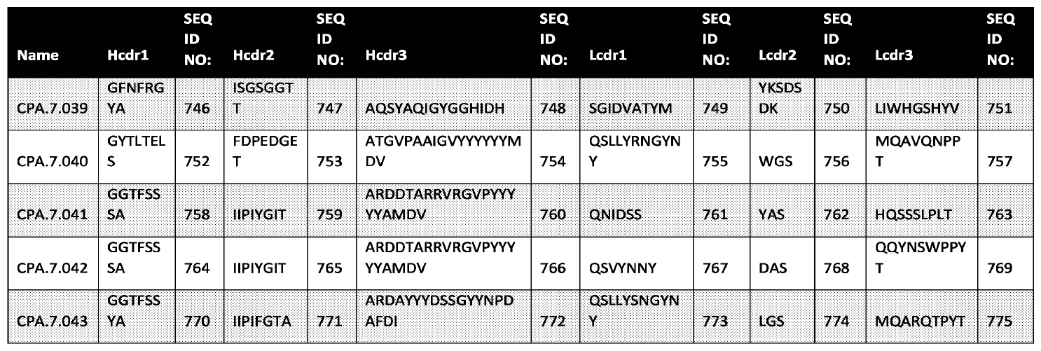

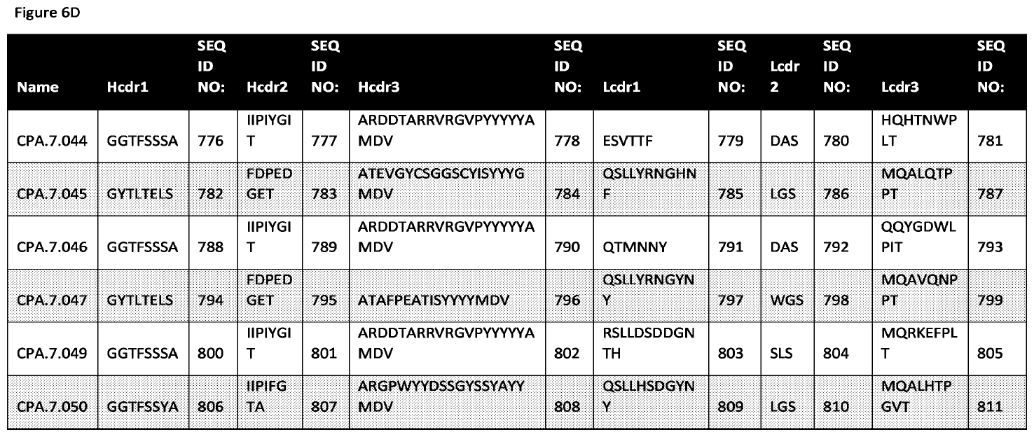

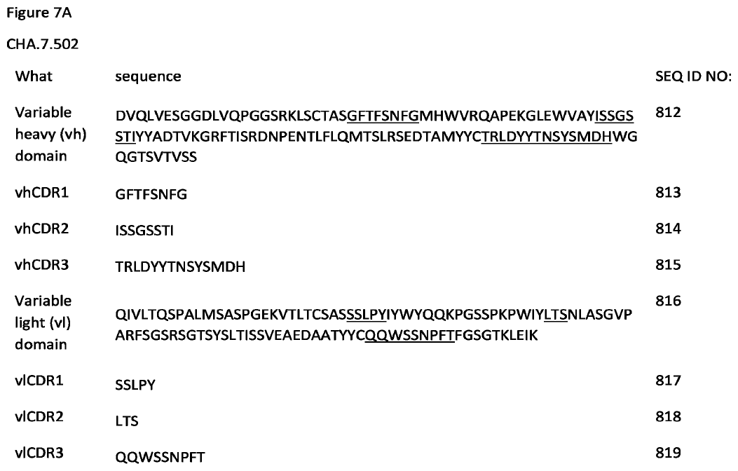

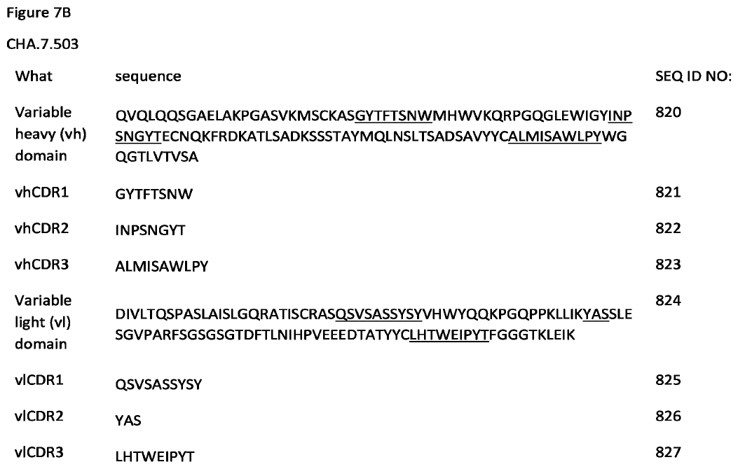

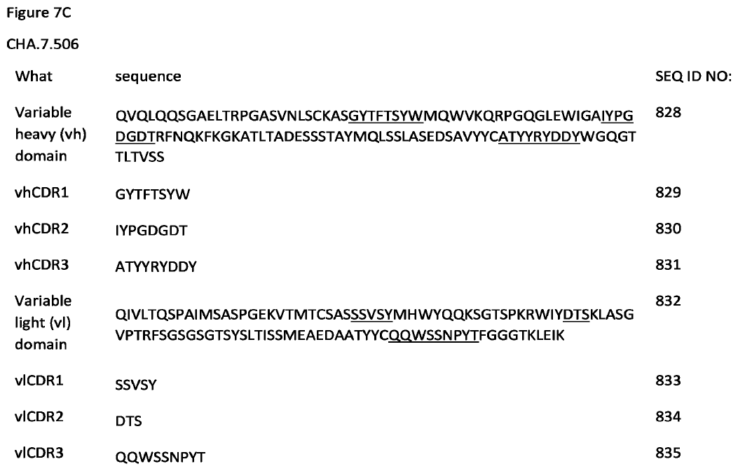

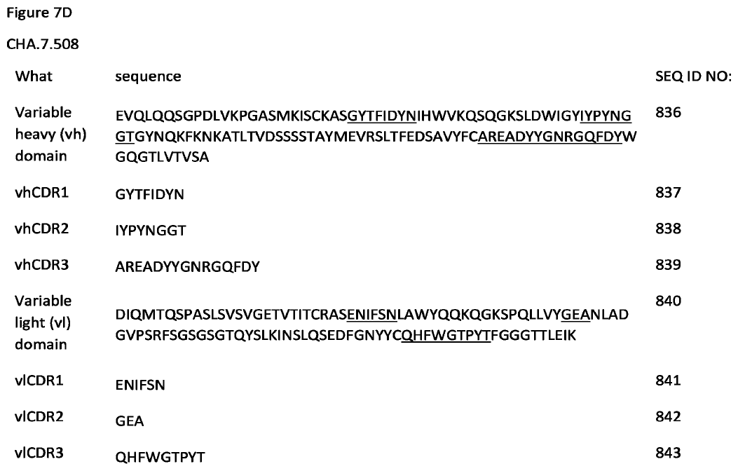

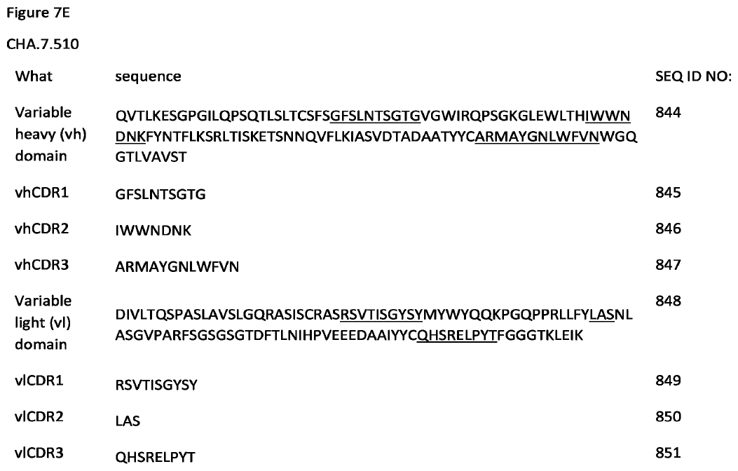

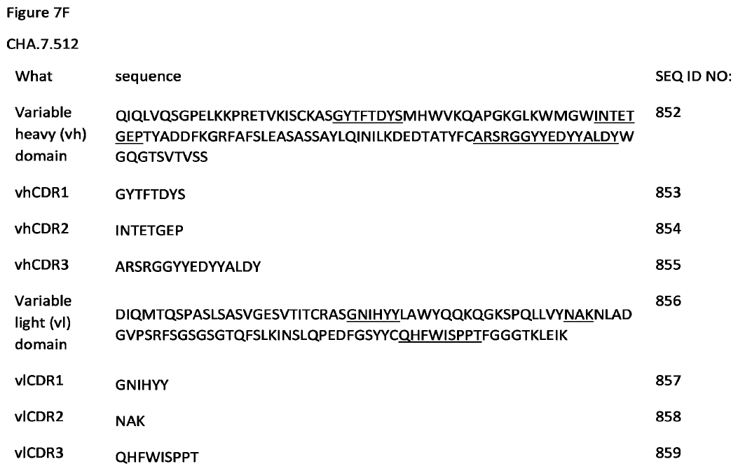

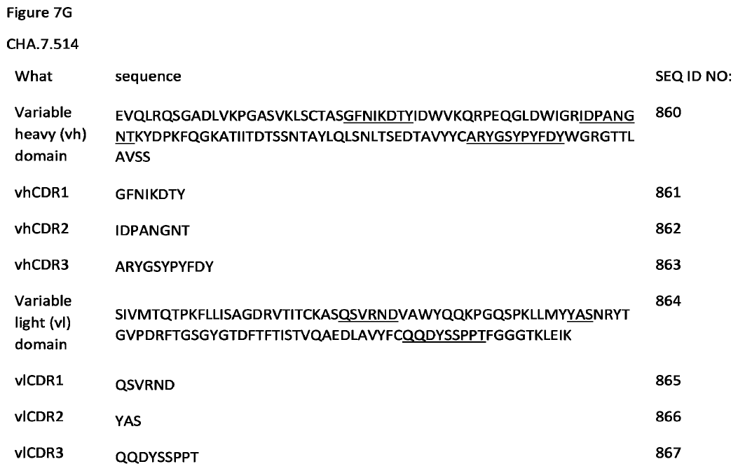

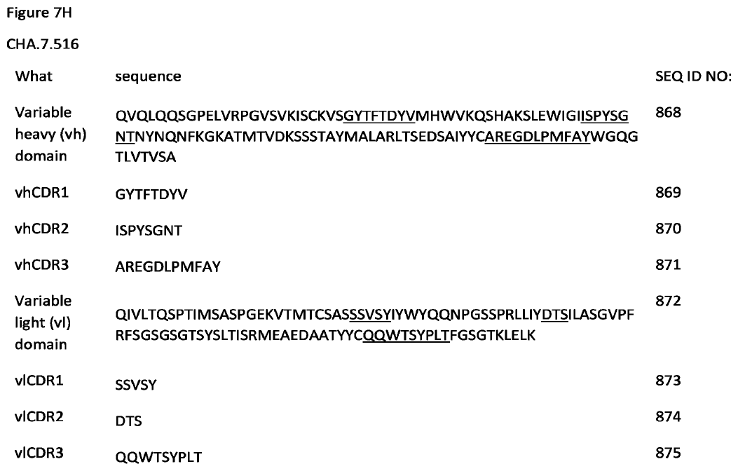

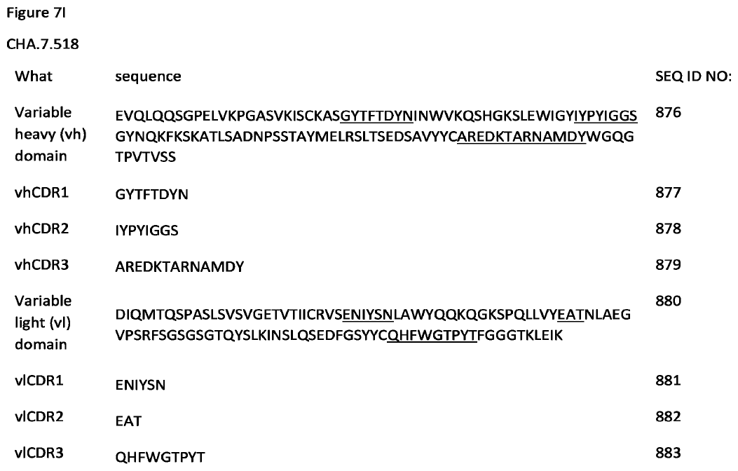

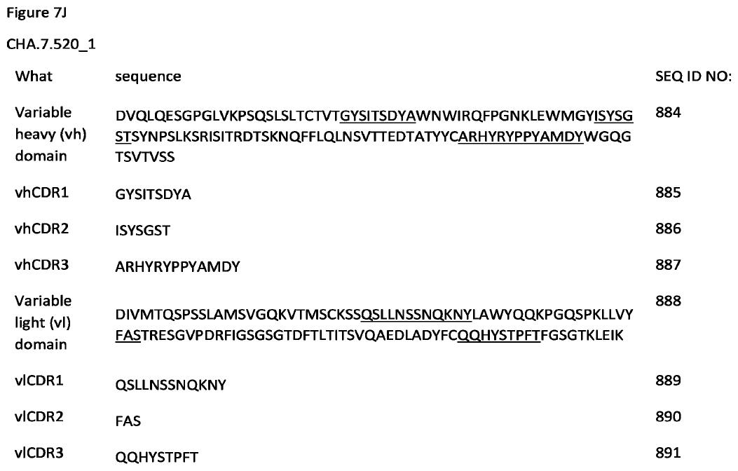

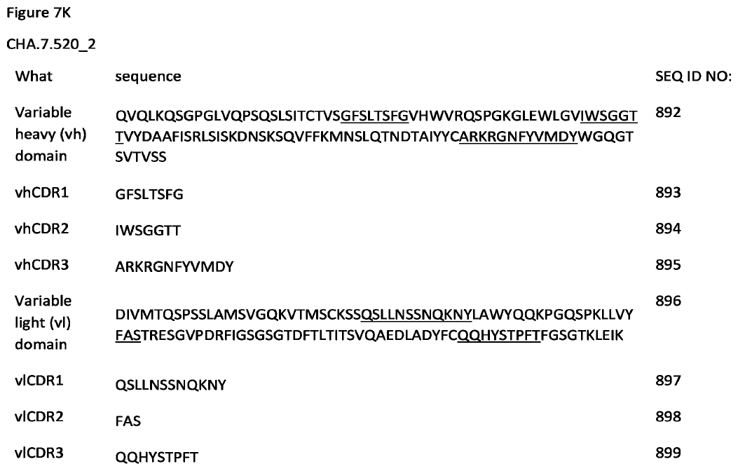

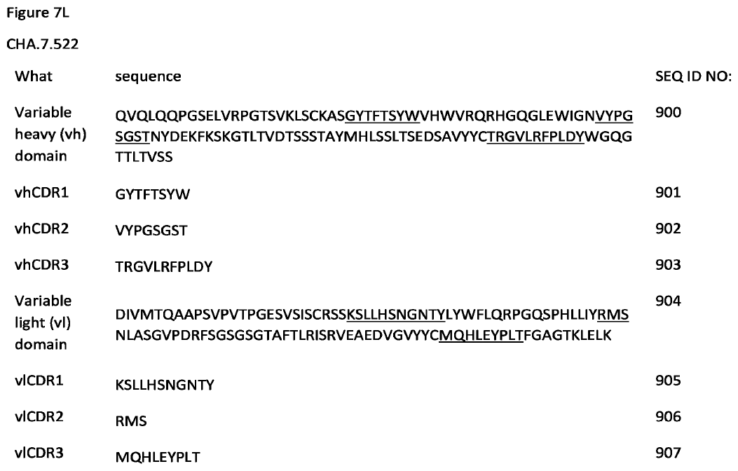

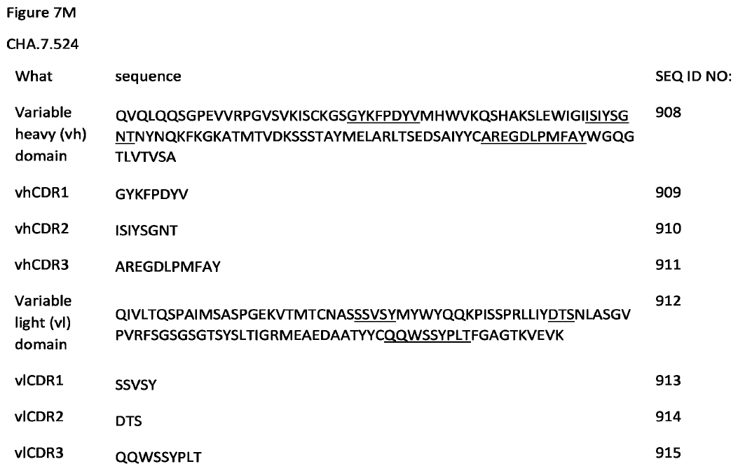

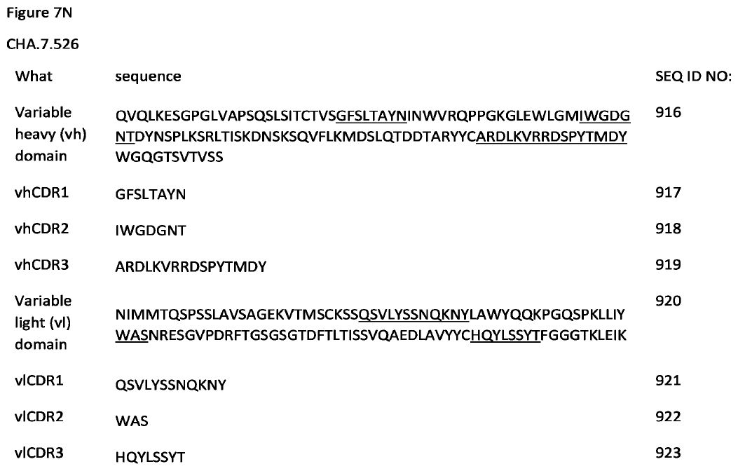

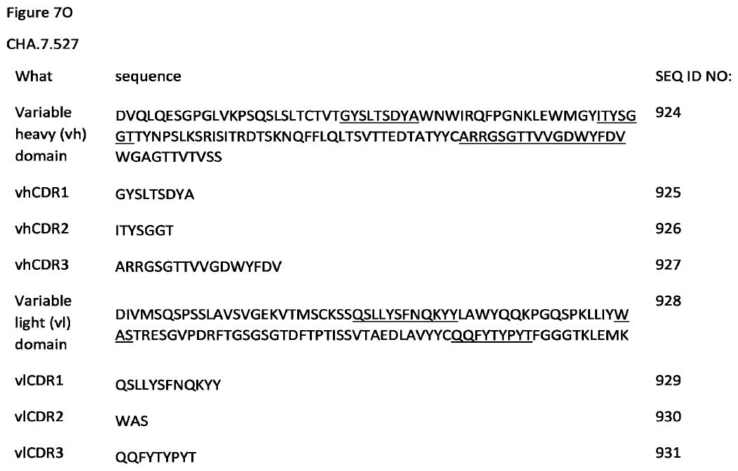

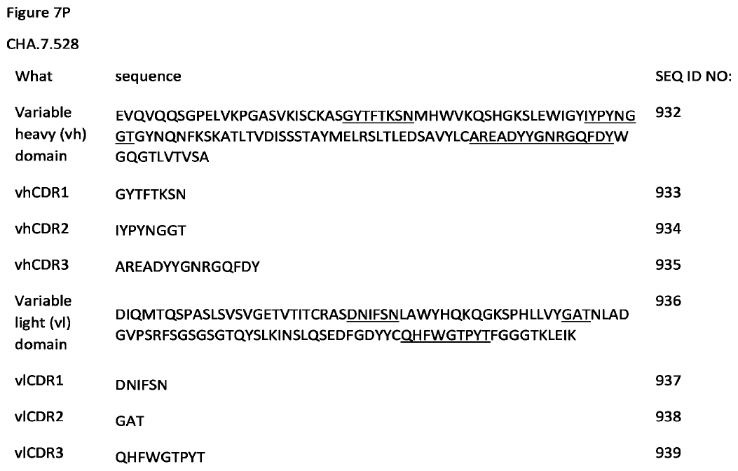

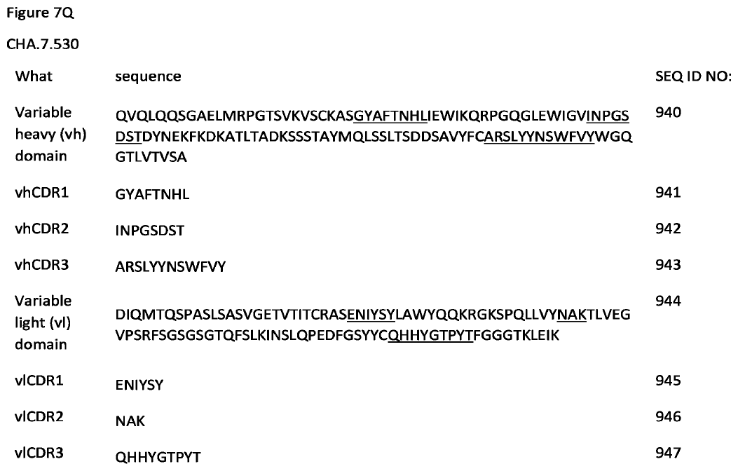

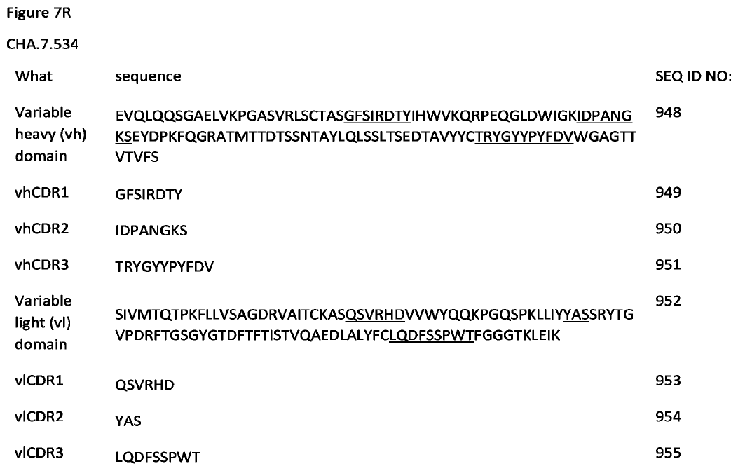

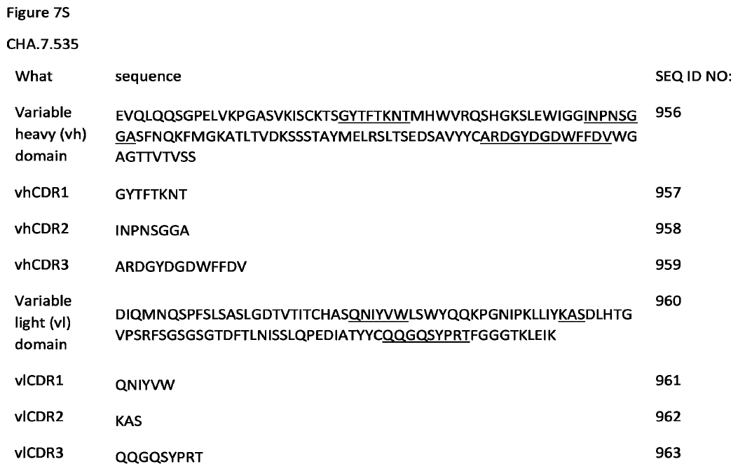

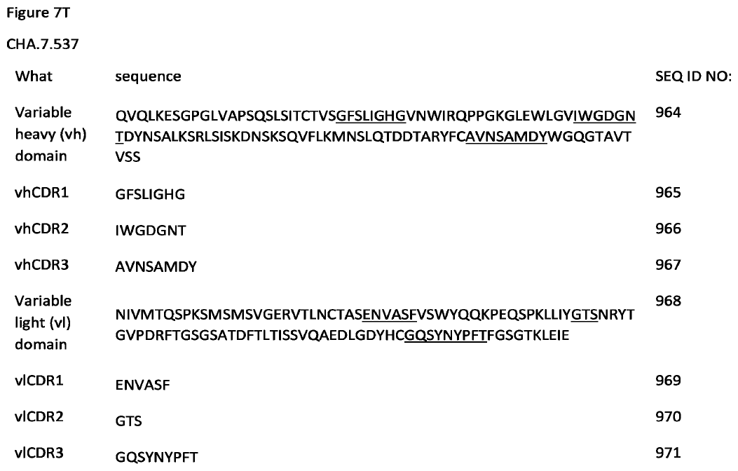

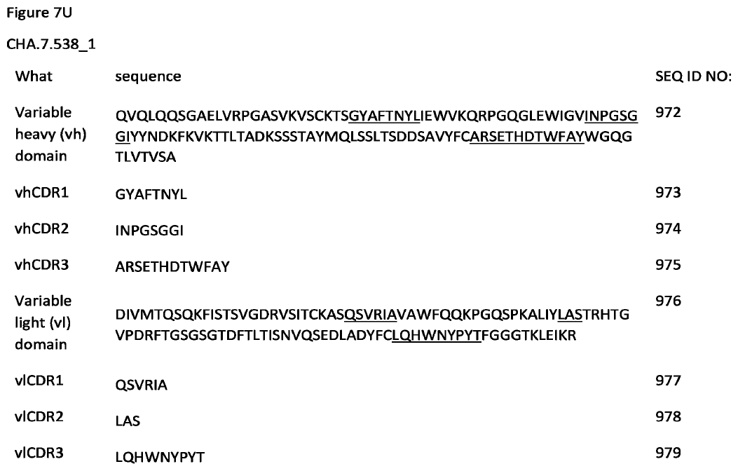

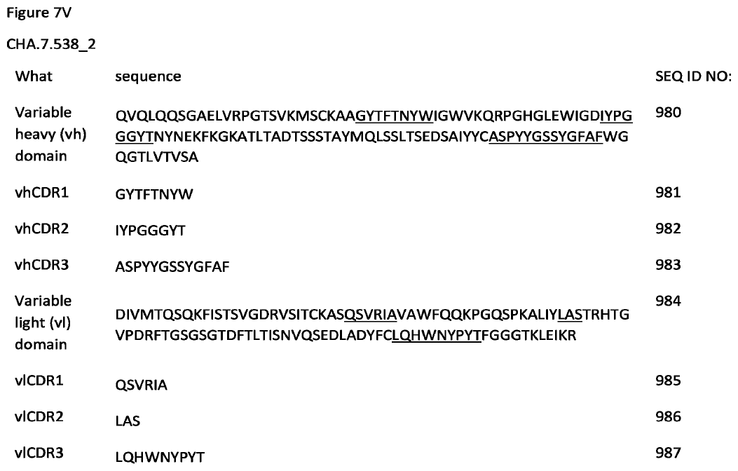

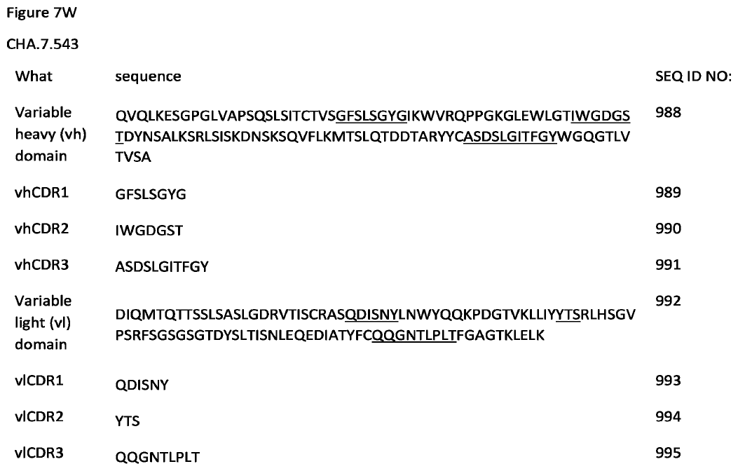

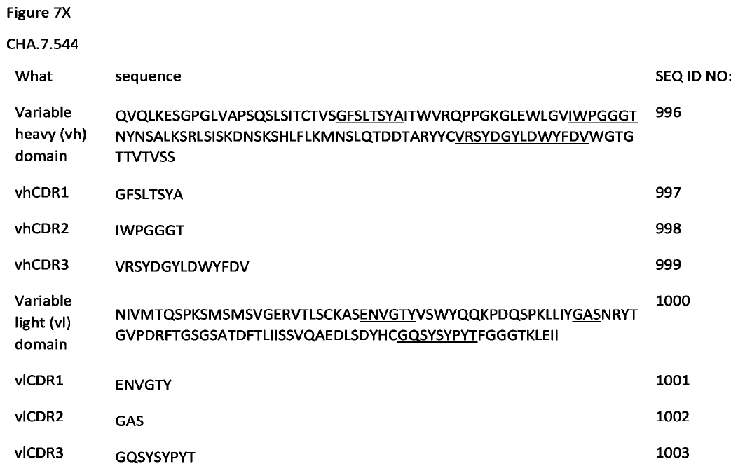

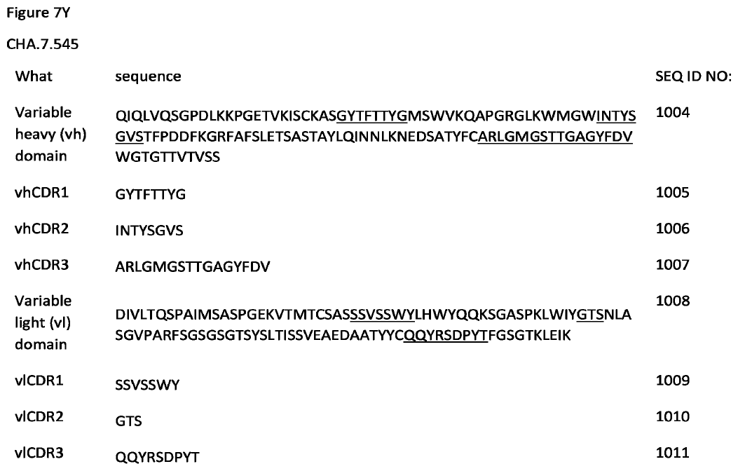

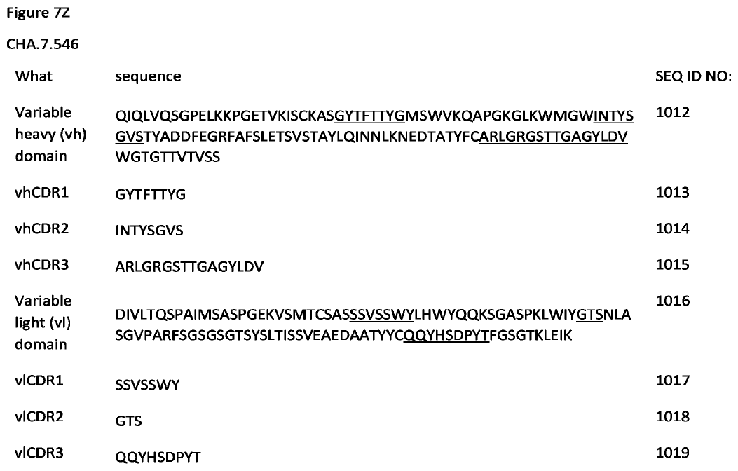

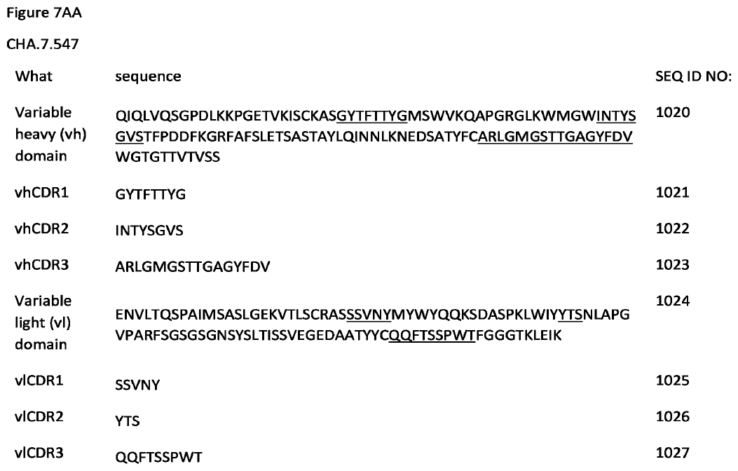

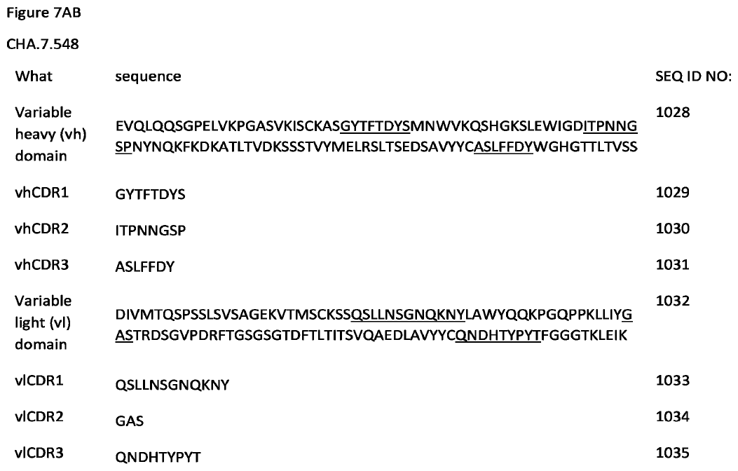

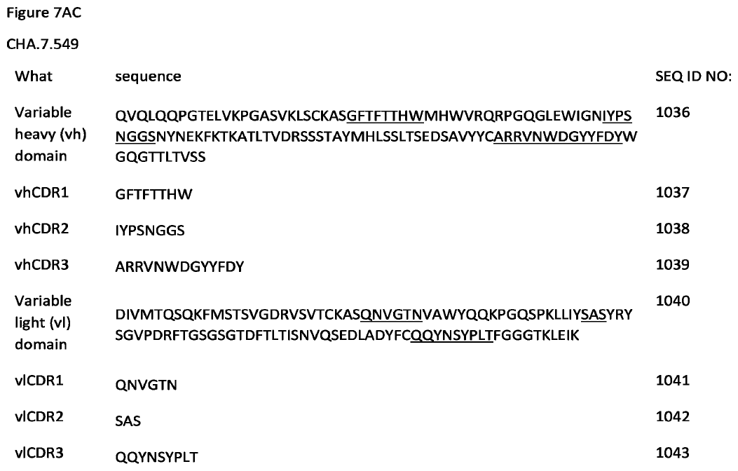

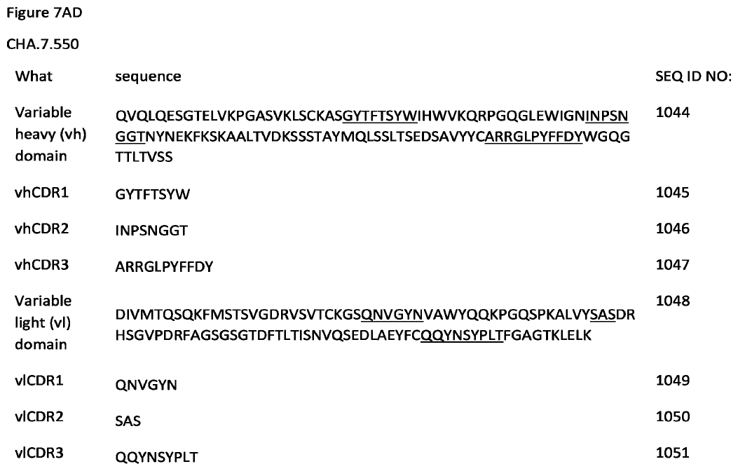

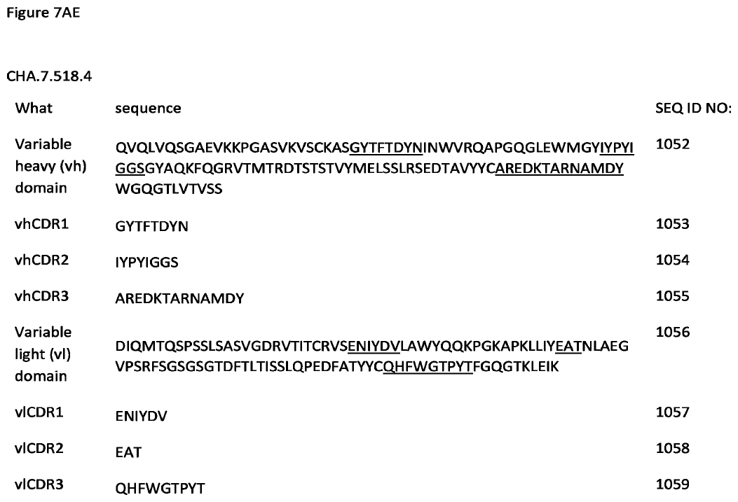

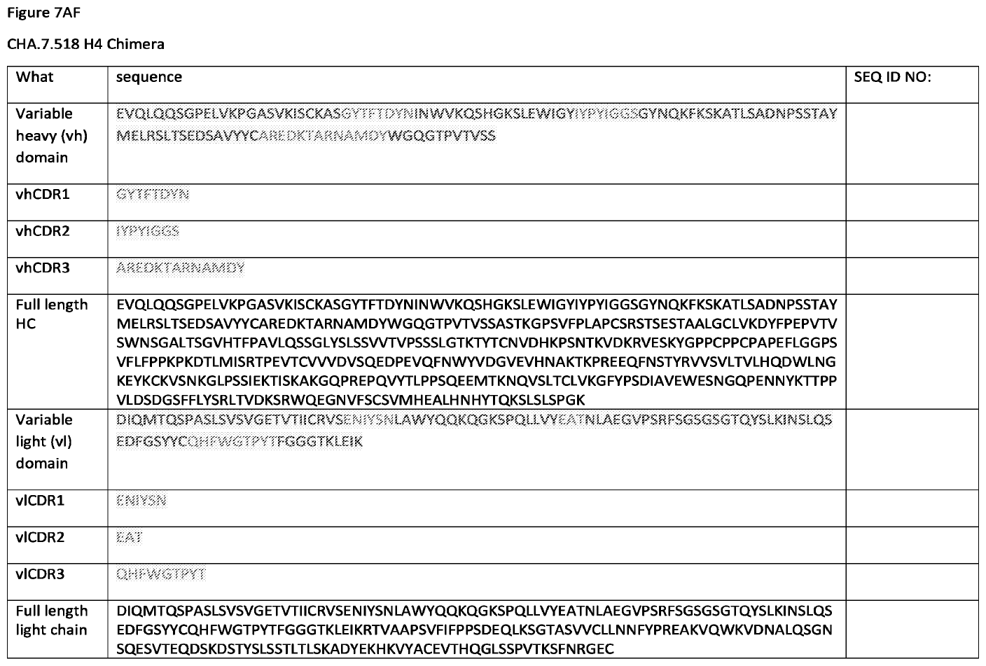

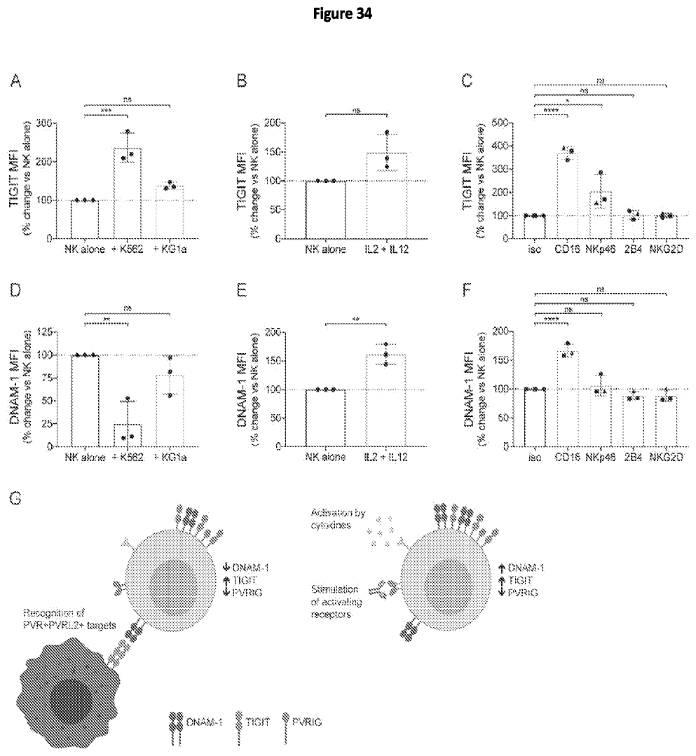

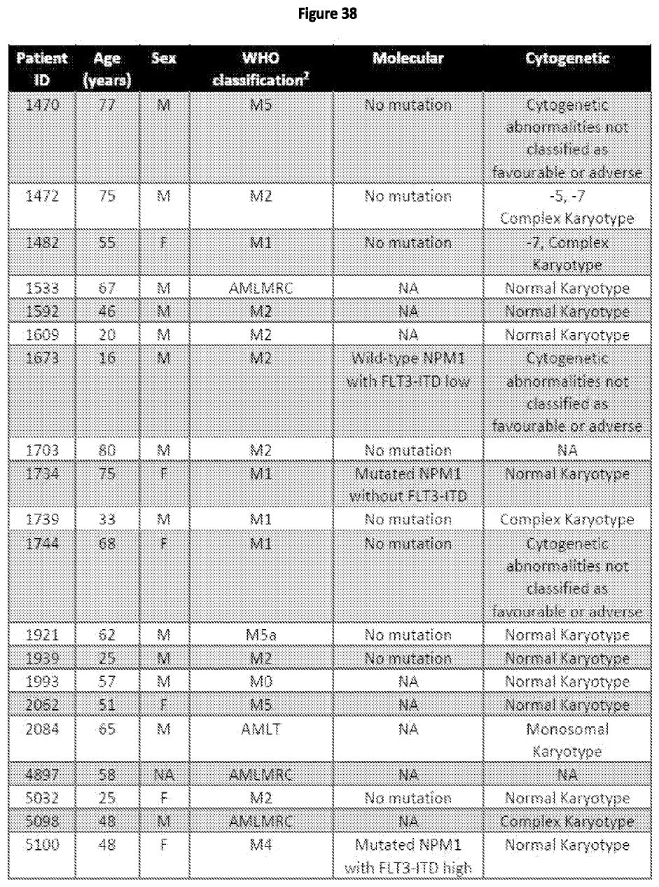

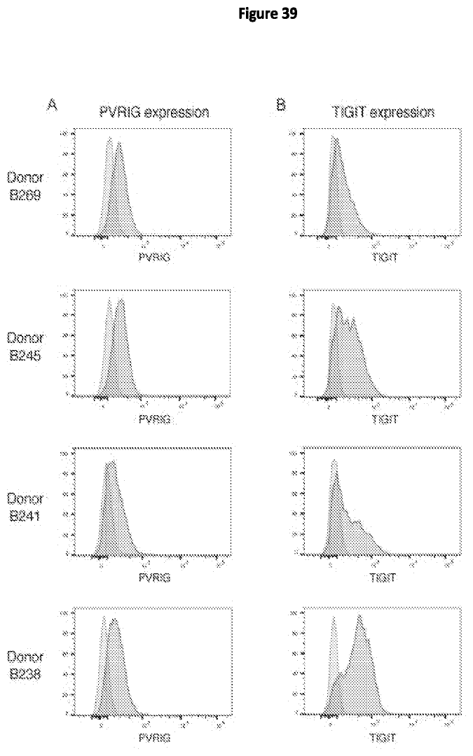

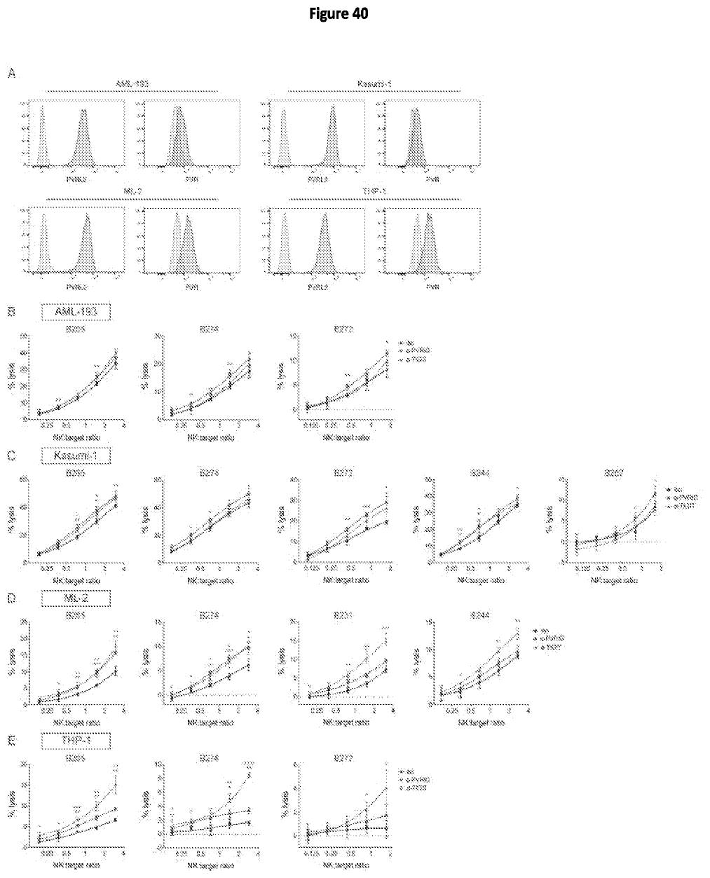

In some embodiments, binding affinity for the anti-PVRIG can be correlated with activity. Antibodies that exhibit the highest maximum signal on T cells can correlate with affinities in the picomolar range. In some embodiments, the anti-PVRIG antibodies and/or the anti-TIGIT antibodies can be useful for T cell-based immunotherapy, which is based in part on their affinity. In some embodiments, the anti-PVRIG antibodies and/or anti-TIGIT antibodies can be useful for NK cell-based immunotherapy, which is based in part on their affinity. Reference is made to antibody sequences from WO2016/134333, hereby incorporated by reference and in particular for the anti-PVRIG antigen binding domains outlined in FIG. 38 (depicting sequences that bind PVRIG and block the interaction of PVRIG and PVRL2; include herein as FIG. 4A-4AA), FIG. 39 (depicting sequences that bind PVRIG and do not block the interaction of PVRIG and PVRL2; included herein as FIG. 5A-5H), FIG. 40A-40T (depicting CDRs and data from these antibodies; included herein as FIG. 6A-6G), and FIG. 41A-41T (depicting CDRs from hybridomas that bind and block; included herein as FIG. 7A-7AE), as well as FIG. 34A-34D providing additional PVRIG binding portions for use in the anti-PVRIG. That is, the Figures and Legends as well as the particular sequences and SEQ ID NO:s from all CPA.7 and CHA.7 antibodies (including CDRs, VH and VL and full length sequences) from WO2016/134333 are expressly incorporated herein.

The anti-PVRIG antibodies for use with the invention have binding affinities (as measured using techniques outlined herein) in the picomolar range, e.g. from 0.1 to 9 pM, with from about 0.2 to about 2 being preferred, and from about 0.2 to about 0.5 being of particular use.

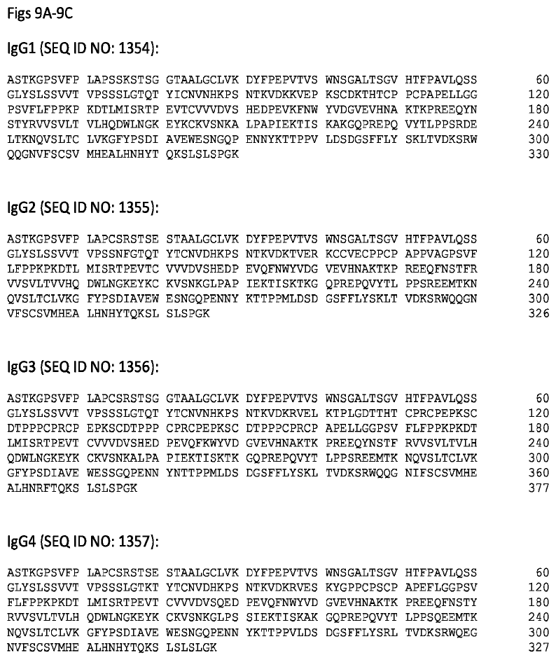

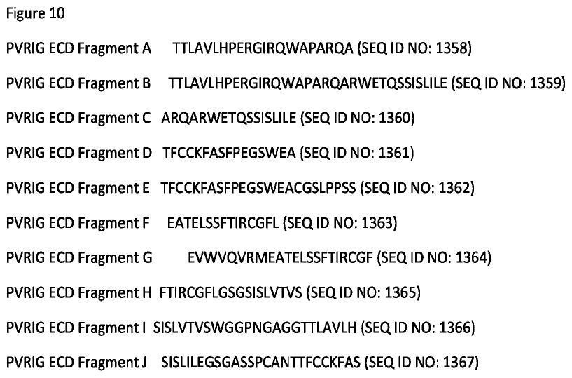

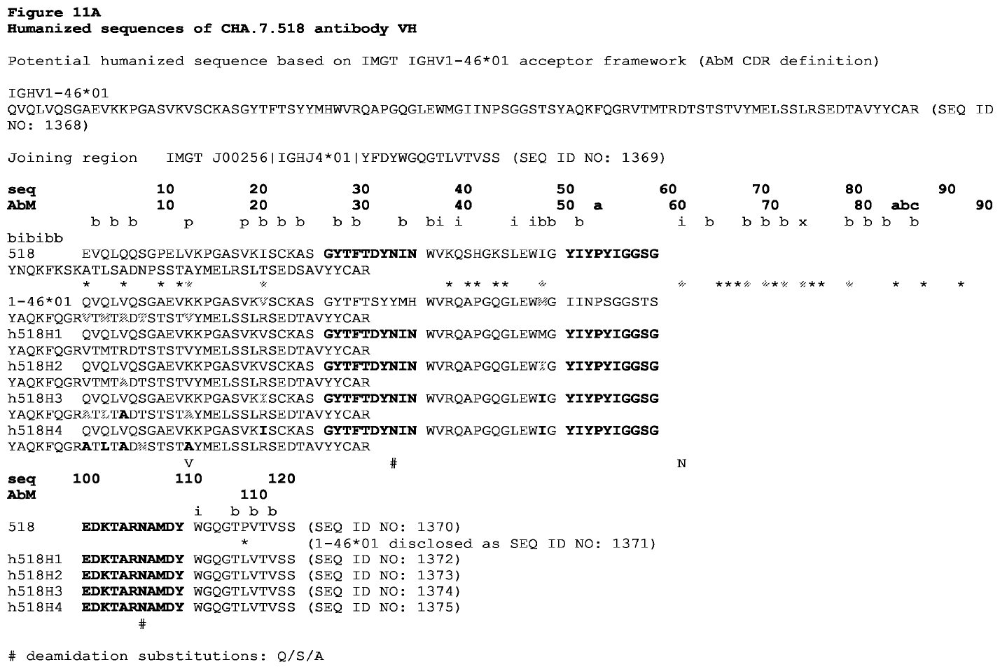

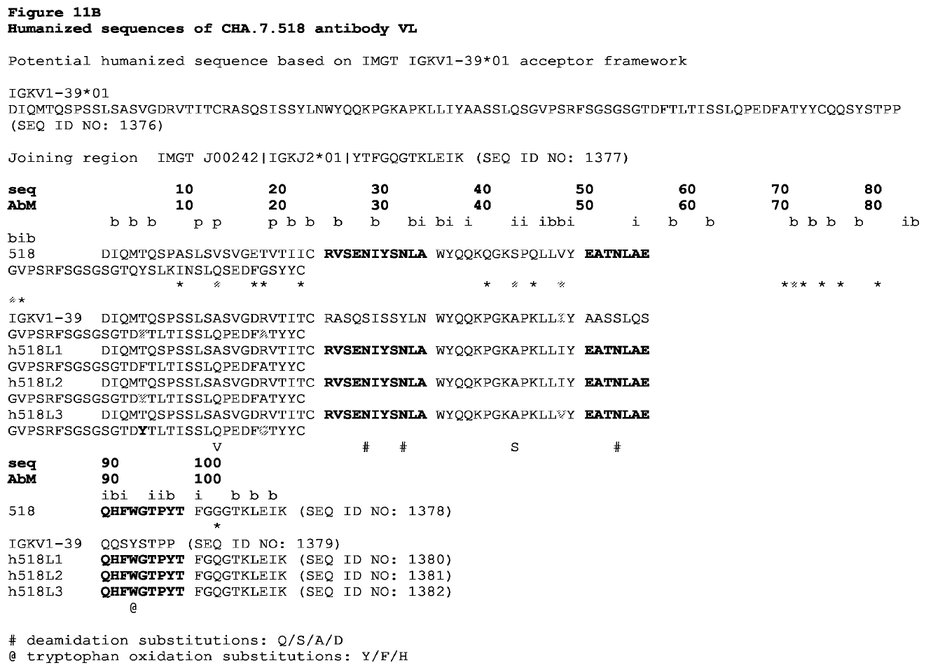

The PVRIG antibodies which can find use in the antibodies of the present invention are labeled as follows. These PVRIG antibodies described herein are labeled as follows. The PVRIG antibodies have reference numbers, for example “CPA.7.013”. This represents the combination of the variable heavy and variable light chains, as depicted in FIG. 4A-4AA and FIGS. 5A-5H for example. “CPA.7.013.VH” refers to the variable heavy portion of CPA.7.013, while “CPA.7.013.VL” is the variable light chain. “CPA.7.013.vhCDR1”, “CPA.7.013.vhCDR2”, “CPA.7.013.vhCDR3”, “CPA.7.013.vlCDR1”, “CPA.7.013.vlCDR2”, and “CPA.7.013.vlCDR3”, refers to the CDRs are indicated. “CPA.7.013.HC” refers to the entire heavy chain (e.g. variable and constant domain) of this molecule, and “CPA.7.013.LC” refers to the entire light light chain (e.g. variable and constant domain) of the same molecule. “CPA.7.013.H1” refers to a full length antibody comprising the variable heavy and light domains, including the constant domain of Human IgG1 (hence, the H1; IgG1, IgG2, IgG3 and IgG4 sequences are shown in FIGS. 9A-9C and 20A-20B). Accordingly, “CPA.7.013.H2” would be the CPA.7.013 variable domains linked to a Human IgG2. “CPA.7.013.H3” would be the CPA.7.013 variable domains linked to a Human IgG3, and “CPA.7.013.H4” would be the CPA.7.013 variable domains linked to a Human IgG4. The anti-PVRIG antibodies and/or anti-TIGIT antibodies for use with the invention can comprise any of the PVRIG antibody sequences and/or PVRIG antigen binding domain sequences as the PVRIG binding portion of the anti-PVRIG antibodies and/or anti-TIGIT antibodies.

The PVRIG antibodies which can find use in the antibodies for use with the invention are labeled as follows. The antibodies have reference numbers, for example “CHA.7.518.1”. This represents the combination of the variable heavy and variable light chains, as depicted in FIG. 7A-7AE, for example, with the understanding that these antibodies include two heavy chains and two light chains. “CPA. 7.518.1.VH” refers to the variable heavy portion of CPA. 7.518.1, while “CPA.7.518.1.VL” is the variable light chain. “CPA. 7.518.1.vhCDR1”, “CPA.7.518.1.vhCDR2”, “CPA. 7.518.1.vhCDR3”, “CPA. 7.518.1.vlCDR1”, “CPA. 7.518.1.vlCDR2”, and “CPA. 7.518.1.vlCDR3”, refers to the CDRs are indicated. “CPA. 7.518.1.HC” refers to the entire heavy chain (e.g. variable and constant domain) of this molecule, and “CPA. 7.518.1.LC” refers to the entire light chain (e.g. variable and constant domain) of the same molecule. In general, the human kappa light chain is used for the constant domain of each phage (or humanized hybridoma) antibody herein, although in some embodiments the lambda light constant domain is used. “CPA. 7.518.1.H1” refers to a full-length antibody comprising the variable heavy and light domains, including the constant domain of Human IgG1 (hence, the H1; IgG1, IgG2, IgG3 and IgG4 sequences are shown in FIG. 20A-20B). Accordingly, “CPA. 7.518.1.H2” would be the CPA. 7.518.1 variable domains linked to a Human IgG2. “CPA. 7.518.1.H3” would be the CPA. 7.518.1 variable domains linked to a Human IgG3, and “CPA. 7.518.1.H4” would be the CPA. 7.518.1 variable domains linked to a Human IgG4. Note that in some cases, the human IgGs may have additional mutations, such are described below, and this can be annotated. For example, in many embodiments, there may be a S241P mutation in the human IgG4, and this can be annotated as “CPA. 7.518.1.H4(S241P)” for example. The human IgG4 sequence with this S241P hinge variant is shown in FIG. 20A-20B. Other potential variants are IgG1(N297A), (or other variants that ablate glycosylation at this site and thus many of the effector functions associated with FcγRIIIa binding), and IgG1(D265A), which reduces binding to FcγR receptors. The anti-PVRIG antibodies for use with the invention can comprise any of the PVRIG antibody sequences as the PVRIG binding portion. The anti-PVRIG antibodies for use with the invention can comprise any of the PVRIG antigen binding domain sequences as the PVRIG binding portion.

The invention further provides variable heavy and light domains as well as full length heavy and light chains, any of which can be employed as part of the PVRIG binding portion of the anti-PVRIG antibodies.

In some embodiments, the invention provides scFvs that bind to PVRIG comprising a variable heavy domain and a variable light domain linked by an scFv linker as outlined above. The VL and VH domains can be in either orientation, e.g. from N- to C-terminus “VH-linker-VL” or “VL-linker-VH”. These are named by their component parts; for example, “scFv-CHA.7.518.1VH-linker-VL” or “scFv-CPA. 7.518.1.VL-linker-VH.” Thus, “scFv-CPA. 7.518.1” can be in either orientation. The anti-PVRIG antibodies and/or anti-TIGIT antibodies for use with the invention can comprise an scFv.

The invention provides antigen binding domains, including full length antibodies, which contain a number of specific, enumerated sets of 6 CDRs. The anti-PVRIG antibodies for use with the invention can comprise any of the sets of 6 CDRs from the PVRIG antibody sequences provided herein in the PVRIG binding portion.

The invention further provides variable heavy and light domains as well as full length heavy and light chains.

In many embodiments, the anti-PVRIG antibodies for use with the invention are human (derived from phage) and block binding of PVRIG and PVLR2. The anti-PVRIG antibodies for use with the invention can comprise a PVRIG antibody and/or antigen binding domain sequence capable of both binding and blocking the receptor-ligand interaction as the PVRIG binding portion. The anti-PVRIG antibodies for use with the invention can comprise the CDRs from a PVRIG antibody sequence capable of both binding and blocking the receptor-ligand interaction as the PVRIG binding portion. The CPA antibodies, as well as the CDR sequences, that both bind and block the receptor-ligand interaction are as below, with their components outlined as well, the sequences for which are shown in FIG. 4A-4AA:

CPA.7.001, CPA.7.001.VH, CPA.7.001.VL, CPA.7.001.HC, CPA.7.001.LC and CPA.7.001.H1, CPA.7.001.H2, CPA.7.001.H3, CPA.7.001.H4; CPA.7.001.vhCDR1, CPA.7.001.vhCDR2, CPA.7.001.vhCDR3, CPA.7.001.vlCDR1, CPA.7.001.vlCDR2, and CPA.7.001.vlCDR3; CPA.7.003, CPA.7.003.VH, CPA.7.003.VL, CPA.7.003.HC, CPA.7.003.LC, CPA.7.003.H1, CPA.7.003.H2, CPA.7.003.H3, CPA.7.003.H4; CPA.7.003.vhCDR1, CPA.7.003.vhCDR2, CPA.7.003.vhCDR3, CPA.7.003.vlCDR1, CPA.7.003.vlCDR2, and CPA.7.003.vlCDR3;

CPA.7.004, CPA.7.004.VH, CPA.7.004.VL, CPA.7.004.HC, CPA.7.004.LC, CPA.7.004.H1, CPA.7.004.H2, CPA.7.004.H3 CPA.7.004.H4; CPA.7.004.vhCDR1, CPA.7.004.vhCDR2, CPA.7.004.vhCDR3, CPA.7.004.vlCDR1, CPA.7.004.vlCDR2, and CPA.7.004.vlCDR3;

CPA.7.006, CPA.7.006.VH, CPA.7.006.VL, CPA.7.006.HC, CPA.7.006.LC, CPA.7.006.H1, CPA.7.006.H2, CPA.7.006.H3 CPA.7.006.H4; CPA.7.006.vhCDR1, CPA.7.006.vhCDR2, CPA.7.006.vhCDR3, CPA.7.006.vlCDR1, CPA.7.006.vlCDR2, and CPA.7.006.vlCDR3;

CPA.7.008, CPA.7.008.VH, CPA.7.008.VL, CPA.7.008.HC, CPA.7.008.LC, CPA.7.008.H1, CPA.7.008.H2, CPA.7.008.H3 CPA.7.008.H4; CPA.7.008.vhCDR1, CPA.7.008.vhCDR2, CPA.7.008.vhCDR3, CPA.7.008.vlCDR1, CPA.7.008.vlCDR2, and CPA.7.008.vlCDR3;

CPA.7.009, CPA.7.009.VH, CPA.7.009.VL, CPA.7.009.HC, CPA.7.009.LC, CPA.7.009.H1, CPA.7.009.H2, CPA.7.009.H3 CPA.7.009.H4; CPA.7.009.vhCDR1, CPA.7.009.vhCDR2, CPA.7.009.vhCDR3, CPA.7.009.vlCDR1, CPA.7.009.vlCDR2, and CPA.7.009.vlCDR3;

CPA.7.010, CPA.7.010.VH, CPA.7.010.VL, CPA.7.010.HC, CPA.7.010.LC, CPA.7.010.H1, CPA.7.010.H2, CPA.7.010.H3 CPA.7.010.H4; CPA.7.010.vhCDR1, CPA.7.010.vhCDR2, CPA.7.010.vhCDR3, CPA.7.010.vlCDR1, CPA.7.010.vlCDR2, and CPA.7.010.vlCDR3;

CPA.7.011, CPA.7.011.VH, CPA.7.011.VL, CPA.7.011.HC, CPA.7.011.LC, CPA.7.011.H1, CPA.7.011.H2, CPA.7.011.H3 CPA.7.011.H4; CPA.7.011.vhCDR1, CPA.7.011.vhCDR2, CPA.7.011.vhCDR3, CPA.7.011.vlCDR1, CPA.7.011.vlCDR2, and CPA.7.011.vlCDR3;

CPA.7.012, CPA.7.012.VH, CPA.7.012.VL, CPA.7.012.HC, CPA.7.012.LC, CPA.7.012.H1, CPA.7.012.H2, CPA.7.012.H3 CPA.7.012.H4; CPA.7.012.vhCDR1, CPA.7.012.vhCDR2, CPA.7.012.vhCDR3, CPA.7.012.vlCDR1, CPA.7.012.vlCDR2, and CPA.7.012.vlCDR3;

CPA.7.013, CPA.7.013.VH, CPA.7.013.VL, CPA.7.013.HC, CPA.7.013.LC, CPA.7.013.H1, CPA.7.013.H2, CPA.7.013.H3 CPA.7.013.H4; CPA.7.013.vhCDR1, CPA.7.013.vhCDR2, CPA.7.013.vhCDR3, CPA.7.013.vlCDR1, CPA.7.013.vlCDR2, and CPA.7.013.vlCDR3;

CPA.7.014, CPA.7.014.VH, CPA.7.014.VL, CPA.7.014.HC, CPA.7.014.LC, CPA.7.014.H1, CPA.7.014.H2, CPA.7.014.H3 CPA.7.014.H4; CPA.7.014.vhCDR1, CPA.7.014.vhCDR2, CPA.7.014.vhCDR3, CPA.7.014.vlCDR1, CPA.7.014.vlCDR2, and CPA.7.014.vlCDR3;

CPA.7.015, CPA.7.015.VH, CPA.7.015.VL, CPA.7.015.HC, CPA.7.015.LC, CPA.7.015.H1, CPA.7.015.H2, CPA.7.015.H3 CPA.7.015.H4; CPA.7.015.vhCDR1, CPA.7.015.vhCDR2, CPA.7.015.vhCDR3, CPA.7.015.vlCDR1, CPA.7.015.vlCDR2, and CPA.7.015.vlCDR3;

CPA.7.017, CPA.7.017.VH, CPA.7.017.VL, CPA.7.017.HC, CPA.7.017.LC, CPA.7.017H1, CPA.7.017.H2, CPA.7.017.H3 CPA.7.017.H4; CPA.7.017.vhCDR1, CPA.7.000171.vhCDR2, CPA.7.017.vhCDR3, CPA.7.017.vlCDR1, CPA.7.017.vlCDR2, and CPA.7.017.vlCDR3;

CPA.7.018, CPA.7.018.VH, CPA.7.018.VL, CPA.7.018.HC, CPA.7.018.LC, CPA.7.018.H1, CPA.7.018.H2, CPA.7.018.H3 CPA.7.018.H4; CPA.7.017.vhCDR1, CPA.7.017.vhCDR2, CPA.7.017.vhCDR3, CPA.7.017.vlCDR1, CPA.7.017.vlCDR2, and CPA.7.017.vlCDR3;

CPA.7.019, CPA.7.019.VH, CPA.7.019.VL, CPA.7.019.HC, CPA.7.019.LC, CPA.7.019.H1, CPA.7.019.H2, CPA.7.019.H3 CPA.7.019.H4; CPA.7.019.vhCDR1, CPA.7.019.vhCDR2, CPA.7.019.vhCDR3, CPA.7.019.vlCDR1, CPA.7.019.vlCDR2, and CPA.7.019.vlCDR3;

CPA.7.021, CPA.7.021.VH, CPA.7.021.VL, CPA.7.021.HC, CPA.7.021.LC, CPA.7.021.H1, CPA.7.021.H2, CPA.7.021.H3 CPA.7.021.H4; CPA.7.021.vhCDR1, CPA.7.021.vhCDR2, CPA.7.021.vhCDR3, CPA.7.021.vlCDR1, CPA.7.021.vlCDR2, and CPA.7.021.vlCDR3;

CPA.7.022, CPA.7.022.VH, CPA.7.022.VL, CPA.7.022.HC, CPA.7.022.LC, CPA.7.022.H1, CPA.7.022.H2, CPA.7.022.H3 CPA.7.022.H4; CPA.7.022.vhCDR1, CPA.7.022.vhCDR2, CPA. 7.002201.vhCDR3, CPA.7.022.vlCDR1, CPA.7.022.vlCDR2, and CPA.7.022.vlCDR3;

CPA.7.023, CPA.7.023.VH, CPA.7.023.VL, CPA.7.023.HC, CPA.7.023.LC, CPA.7.023.H1, CPA.7.023.H2, CPA.7.023.H3 CPA.7.023.H4; CPA.7.023.vhCDR1, CPA.7.023.vhCDR2, CPA.7.023.vhCDR3, CPA.7.023.vlCDR1, CPA.7.023.vlCDR2, and CPA.7.023.vlCDR3;

CPA.7.024, CPA.7.024.VH, CPA.7.024.VL, CPA.7.024.HC, CPA.7.024.LC, CPA.7.024.H1, CPA.7.024.H2, CPA.7.024.H3 CPA.7.024.H4; CPA.7.024.vhCDR1, CPA.7.024.vhCDR2, CPA.7.024.vhCDR3, CPA.7.024.vlCDR1, CPA.7.024.vlCDR2, and CPA.7.024.vlCDR3;

CPA.7.033, CPA.7.033.VH, CPA.7.033.VL, CPA.7.033.HC, CPA.7.033.LC, CPA.7.033.H1, CPA.7.033.H2, CPA.7.033.H3 CPA.7.033.H4; CPA.7.033.vhCDR1, CPA.7.033.vhCDR2, CPA.7.033.vhCDR3, CPA.7.033.vlCDR1, CPA.7.033.vlCDR2, and CPA.7.033.vlCDR3;

CPA.7.034, CPA.7.034.VH, CPA.7.034.VL, CPA.7.034.HC, CPA.7.034.LC, CPA.7.034.H1, CPA.7.034.H2, CPA.7.034.H3 CPA.7.034.H4; CPA.7.034.vhCDR1, CPA.7.034.vhCDR2, CPA.7.034.vhCDR3, CPA.7.034.vlCDR1, CPA.7.034.vlCDR2, and CPA.7.034.vlCDR3;

CPA.7.036, CPA.7.036.VH, CPA.7.036.VL, CPA.7.036.HC, CPA.7.036.LC, CPA.7.036.H1, CPA.7.036.H2, CPA.7.036.H3 CPA.7.036.H4; CPA.7.036.vhCDR1, CPA.7.036.vhCDR2, CPA.7.036.vhCDR3, CPA.7.036.vlCDR1, CPA.7.036.vlCDR2, and CPA.7.036.vlCDR3;

CPA.7.040, CPA.7.040.VH, CPA.7.040.VL, CPA.7.040.HC, CPA.7.040.LC, CPA.7.040.H1, CPA.7.040.H2, CPA.7.040.H3 and CPA.7.040.H4; CPA.7.040.vhCDR1, CPA.7.040.vhCDR2, CPA.7.040.vhCDR3, CPA.7.040.vlCDR1, CPA.7.040.vlCDR2, and CPA.7.040.vlCDR3;

CPA.7.046, CPA.7.046.VH, CPA.7.046.VL, CPA.7.046.HC, CPA.7.046.LC, CPA.7.046.H1, CPA.7.046.H2, CPA.7.046.H3 CPA.7.046.H4; CPA.7.046.vhCDR1, CPA.7.046.vhCDR2, CPA.7.046.vhCDR3, CPA.7.046.vlCDR1, CPA.7.046.vlCDR2, and CPA.7.046.vlCDR3;

CPA.7.047, CPA.7.047.VH, CPA.7.047.VL, CPA.7.047.HC, CPA.7.047.LC, CPA.7.047.H1, CPA.7.047.H2, CPA.7.047.H3 CPA.7.047.H4; CPA.7.047.vhCDR1, CPA.7.047.vhCDR2, CPA.7.047.vhCDR3, CPA.7.047.vlCDR1, CPA. 7.004701.vlCDR2, and CPA.7.047.vlCDR3;

CPA.7.049, CPA.7.049.VH, CPA.7.049.VL, CPA.7.049.HC, CPA.7.049.LC, CPA.7.049.H1, CPA.7.049.H2, CPA.7.049.H3 CPA.7.049.H4; CPA.7.049.vhCDR1, CPA.7.049.vhCDR2, CPA.7.049.vhCDR3, CPA.7.049.vlCDR1, CPA.7.049.vlCDR2, and CPA.7.049.vlCDR3; and

CPA.7.050, CPA.7.050.VH, CPA.7.050.VL, CPA.7.050.HC, CPA.7.050.LC, CPA.7.050.H1, CPA.7.050.H2, CPA.7.050.H3 CPA.7.050.H4, CPA.7.050.vhCDR1, CPA.7.050.vhCDR2, CPA.7.050.vhCDR3, CPA.7.050.vlCDR1, CPA.7.050.vlCDR2, and CPA.7.050.vlCDR3.

In addition, there are a number of CPA antibodies generated herein that bound to PVRIG but did not block the interaction of PVRIG and PVLR2. The anti-PVRIG antibodies for use with the invention can comprise a PVRIG antibody and/or antigen binding domain sequence capable of binding but not blocking the receptor-ligand interaction as the PVRIG binding portion. The anti-PVRIG antibodies for use with the invention can comprise the CDRs from a PVRIG antibody sequence capable of sequence capable of binding but not blocking the receptor-ligand interaction as the PVRIG binding portion.

The CPA antibodies, as well as the CDR sequences, that bind but do not block the receptor-ligand interaction are as below, with their components outlined as well, the sequences for which are shown in FIG. 4A-4AA:

CPA.7.028, CPA.7.028.VH, CPA.7.028.VL, CPA.7.028.HC, CPA.7.028.LC, CPA.7.028.H1, CPA.7.028.H2, CPA.7.028.H3 and CPA.7.028.H4; CPA.7.028.vhCDR1, CPA.7.028.vhCDR2, CPA.7.028.vhCDR3, CPA.7.028.vlCDR1, CPA.7.028.vlCDR2, and CPA.7.028.vlCDR3.

CPA.7.030, CPA.7.030.VH, CPA.7.030.VL, CPA.7.030.HC, CPA.7.030.LC, CPA.7.030.H1, CPA.7.030.H2, CPA.7.030.H3 and CPA.7.030.H4; CPA.7.030.vhCDR1, CPA.7.030.vhCDR2, CPA.7.030.vhCDR3, CPA.7.030.vlCDR1, CPA.7.030.vlCDR2, and CPA.7.030.vlCDR3.

CPA.7.041, CPA.7.041.VH, CPA.7.041.VL, CPA.7.041.HC, CPA.7.041.LC, CPA.7.041.H1, CPA.7.041.H2, CPA.7.041.H3 and CPA.7.041.H4; CPA.7.041.vhCDR1, CPA.7.041.vhCDR2, CPA.7.041.vhCDR3, CPA.7.041.vlCDR1, CPA.7.041.vlCDR2, and CPA.7.041.vlCDR3.

CPA.7.016, CPA.7.016.VH, CPA.7.016.VL, CPA.7.016.HC, CPA.7.016.LC, CPA.7.016.H1, CPA.7.016.H2, CPA.7.016.H3 and CPA.7.016.H4; CPA.7.016.vhCDR1, CPA.7.016.vhCDR2, CPA.7.016.vhCDR3, CPA.7.016.vlCDR1, CPA.7.016.vlCDR2, and CPA.7.016.vlCDR3.

CPA.7.020, CPA.7.020.VH, CPA.7.020.VL, CPA.7.020.HC, CPA.7.020.LC, CPA.7.020.H1, CPA.7.020.H2, CPA.7.020.H3 and CPA.7.020.H4; CPA.7.020.vhCDR1, CPA.7.020.vhCDR2, CPA.7.020.vhCDR3, CPA.7.020.vlCDR1, CPA.7.020.vlCDR2, and CPA.7.020.vlCDR3.

CPA.7.038, CPA.7.038.VH, CPA.7.038.VL, CPA.7.038.HC, CPA.7.038.LC, CPA.7.038.H1, CPA.7.038.H2, CPA.7.038.H3 and CPA.7.038.H4; CPA.7.038.vhCDR1, CPA.7.038.vhCDR2, CPA.7.038.vhCDR3, CPA.7.038.vlCDR1, CPA.7.038.vlCDR2, and CPA.7.038.vlCDR3.

CPA.7.044, CPA.7.044.VH, CPA.7.044.VL, CPA.7.044.HC, CPA.7.044.LC, CPA.7.044.H1, CPA.7.044.H2, CPA.7.044.H3 and CPA.7.044.H4; CPA.7.044.vhCDR1, CPA.7.044.vhCDR2, CPA.7.044.vhCDR3, CPA.7.044.vlCDR1, CPA.7.044.vlCDR2, and CPA.7.044.vlCDR3.

CPA.7.045, CPA.7.045.VH, CPA.7.045.VL, CPA.7.045.HC, CPA.7.045.LC, CPA.7.045.H1, CPA.7.045.H2, CPA.7.045.H3 and CPA.7.045.H4; CPA.7.045.vhCDR1, CPA.7.045.vhCDR2, CPA.7.045.vhCDR3, CPA.7.045.vlCDR1, CPA.7.045.vlCDR2, and CPA.7.045.vlCDR3.

As discussed herein, the invention further provides variants of the above components, including variants in the CDRs, as outlined above. In addition, variable heavy chains can be 80%, 90%, 95%, 98% or 99% identical to the “VH” sequences herein, and/or contain from 1, 2, 3, 4, 5, 6, 7, 8, 9, 10 amino acid changes, or more, when Fc variants are used. Variable light chains are provided that can be 80%, 90%, 95%, 98% or 99% identical to the “VL” sequences herein, and/or contain from 1, 2, 3, 4, 5, 6, 7, 8, 9, 10 amino acid changes, or more, when Fc variants are used. Similarly, heavy and light chains are provided that are 80%, 90%, 95%, 98% or 99% identical to the “HC” and “LC” sequences herein, and/or contain from 1, 2, 3, 4, 5, 6, 7, 8, 9, 10 amino acid changes, or more, when Fc variants are used. The anti-PVRIG antibodies for use with the invention can comprise any of these PVRIG antibody and/or antigen binding domain sequences described herein.

Furthermore, the present invention provides a number of CHA antibodies, which are murine antibodies generated from hybridomas. As is well known the art, the six CDRs are useful when put into either human framework variable heavy and variable light regions or when the variable heavy and light domains are humanized.

The anti-PVRIG antibodies for use with the invention can comprise any of the following CHA sets of CDRs from PVRIG antibody sequences as part of the PVRIG binding portion. Accordingly, the present invention provides anti-PVRIG antibodies, that comprise the following CHA sets of CDRs as part of the PVRIG binding portion of the anti-PVRIG antibody, the sequences of which are shown in FIG. 7A-7AE:

CHA.7.502.vhCDR1, CHA.7.502.vhCDR2, CHA.7.502.vhCDR3, CHA.7.502.vlCDR1, CHA.7.502.vlCDR2, and CHA.7.502.vlCDR3.

CHA.7.503.vhCDR1, CHA.7.503.vhCDR2, CHA.7.503.vhCDR3, CHA.7.503.vlCDR1, CHA.7.503.vlCDR2, and CHA.7.503.vlCDR3.

CHA.7.506.vhCDR1, CHA.7.506.vhCDR2, CHA.7.506.vhCDR3, CHA.7.506.vlCDR1, CHA.7.506.vlCDR2, and CHA.7.506.vlCDR3.

CHA.7.508.vhCDR1, CHA.7.508.vhCDR2, CHA.7.508.vhCDR3, CHA.7.508.vlCDR1, CHA.7.508.vlCDR2, and CHA.7.508.vlCDR3.

CHA.7.510.vhCDR1, CHA.7.510.vhCDR2, CHA.7.510.vhCDR3, CHA.7.510.vlCDR1, CHA.7.510.vlCDR2, and CHA.7.510.vlCDR3.

CHA.7.512.vhCDR1, CHA.7.512.vhCDR2, CHA.7.512.vhCDR3, CHA.7.512.vlCDR1, CHA.7.512.vlCDR2, and CHA.7.512.vlCDR3.

CHA.7.514.vhCDR1, CHA.7.514.vhCDR2, CHA.7.514.vhCDR3, CHA.7.514.vlCDR1, CHA.7.514.vlCDR2, and CHA.7.514.vlCDR3.

CHA.7.516.vhCDR1, CHA.7.516.vhCDR2, CHA.7.516.vhCDR3, CHA.7.516.vlCDR1, CHA.7.516.vlCDR2, and CHA.7.516.vlCDR3.

CHA.7.518.vhCDR1, CHA.7.518.vhCDR2, CHA.7.518.vhCDR3, CHA.7.518.vlCDR1, CHA.7.518.vlCDR2, and CHA.7.518.vlCDR3.

CHA.7.520_1.vhCDR1, CHA.7.520_1.vhCDR2, CHA.7.520_1.vhCDR3, CHA.7.520_1.vlCDR1, CHA.7.520_1.vlCDR2, and CHA.7.520_1.vlCDR3.

CHA.7.520_2.vhCDR1, CHA.7.520_2.vhCDR2, CHA.7.520_2.vhCDR3, CHA.7.520_2.vlCDR1, CHA.7.520_2.vlCDR2, and CHA.7.520_2.vlCDR3.

CHA.7.522.vhCDR1, CHA.7.522.vhCDR2, CHA.7.522.vhCDR3, CHA.7.522.vlCDR1, CHA.7.522.vlCDR2, and CHA.7.522.vlCDR3.

CHA.7.524.vhCDR1, CHA.7.524.vhCDR2, CHA.7.524.vhCDR3, CHA.7.524.vlCDR1, CHA.7.524.vlCDR2, and CHA.7.524.vlCDR3.

CHA.7.526.vhCDR1, CHA.7.526.vhCDR2, CHA.7.526.vhCDR3, CHA.7.526.vlCDR1, CHA.7.526.vlCDR2, and CHA.7.526.vlCDR3.

CHA.7.527.vhCDR1, CHA.7.527.vhCDR2, CHA.7.527.vhCDR3, CHA.7.527.vlCDR1, CHA.7.527.vlCDR2, and CHA.7.527.vlCDR3.

CHA.7.528.vhCDR1, CHA.7.528.vhCDR2, CHA.7.528.vhCDR3, CHA.7.528.vlCDR1, CHA.7.528.vlCDR2, and CHA.7.528.vlCDR3.

CHA.7.530.vhCDR1, CHA.7.530.vhCDR2, CHA.7.530.vhCDR3, CHA.7.530.vlCDR1, CHA.7.530.vlCDR2, and CHA.7.530.vlCDR3.

CHA.7.534.vhCDR1, CHA.7.534.vhCDR2, CHA.7.534.vhCDR3, CHA.7.534.vlCDR1, CHA.7.534.vlCDR2, and CHA.7.534.vlCDR3.

CHA.7.535.vhCDR1, CHA.7.535.vhCDR2, CHA.7.535.vhCDR3, CHA.7.535.vlCDR1, CHA.7.535.vlCDR2, and CHA.7.535.vlCDR3.

CHA.7.537.vhCDR1, CHA.7.537.vhCDR2, CHA.7.537.vhCDR3, CHA.7.537.vlCDR1, CHA.7.537.vlCDR2, and CHA.7.537.vlCDR3.

CHA.7.538_1.vhCDR1, CHA.7.538_1.vhCDR2, CHA.7.538_1.vhCDR3, CHA.7.538_1.vlCDR1, CHA.7.538_1.vlCDR2, and CHA.7.538_1.vlCDR3.

CHA.7.538_2.vhCDR1, CHA.7.538_2.vhCDR2, CHA.7.538_2.vhCDR3, CHA.7.538_2.vlCDR1, CHA.7.538_2.vlCDR2, and CHA.7.538_2.vlCDR3.

CHA.7.543.vhCDR1, CHA.7.543.vhCDR2, CHA.7.543.vhCDR3, CHA.7.543.vlCDR1, CHA.7.543.vlCDR2, and CHA.7.543.vlCDR3.

CHA.7.544.vhCDR1, CHA.7.544.vhCDR2, CHA.7.544.vhCDR3, CHA.7.544.vlCDR1, CHA.7.544.vlCDR2, and CHA.7.544.vlCDR3.

CHA.7.545.vhCDR1, CHA.7.545.vhCDR2, CHA.7.545.vhCDR3, CHA.7.545.vlCDR1, CHA.7.545.vlCDR2, and CHA.7.545.vlCDR3.

CHA.7.546.vhCDR1, CHA.7.546.vhCDR2, CHA.7.546.vhCDR3, CHA.7.546.vlCDR1, CHA.7.546.vlCDR2, and CHA.7.546.vlCDR3.

CHA.7.547.vhCDR1, CHA.7.547.vhCDR2, CHA.7.547.vhCDR3, CHA.7.547.vlCDR1, CHA.7.547.vlCDR2, and CHA.7.547.vlCDR3.

CHA.7.548.vhCDR1, CHA.7.548.vhCDR2, CHA.7.548.vhCDR3, CHA.7.548.vlCDR1, CHA.7.548.vlCDR2, and CHA.7.548.vlCDR3.

CHA.7.549.vhCDR1, CHA.7.549.vhCDR2, CHA.7.549.vhCDR3, CHA.7.549.vlCDR1, CHA.7.549.vlCDR2, and CHA.7.549.vlCDR3.

CHA.7.550.vhCDR1, CHA.7.550.vhCDR2, CHA.7.550.vhCDR3, CHA.7.550.vlCDR1, CHA.7.550.vlCDR2, and CHA.7.550.vlCDR3.

CHA.7.518.4.vhCDR1, CHA.7.518.4.vhCDR2, CHA.7.518.4.vhCDR3, CHA.7.518.4.vlCDR1, CHA.7.518.4.vlCDR2, and CHA.7.518.4.vlCDR3.

As above, these sets of CDRs may also be amino acid variants as described above.

In addition, the framework regions of the variable heavy and variable light chains can be humanized as is known in the art (with occasional variants generated in the CDRs as needed), and thus humanized variants of the VH and VL chains of FIGS. 7A-7DD can be generated. Furthermore, the humanized variable heavy and light domains can then be fused with human constant regions, such as the constant regions from IgG1, IgG2, IgG3 and IgG4.

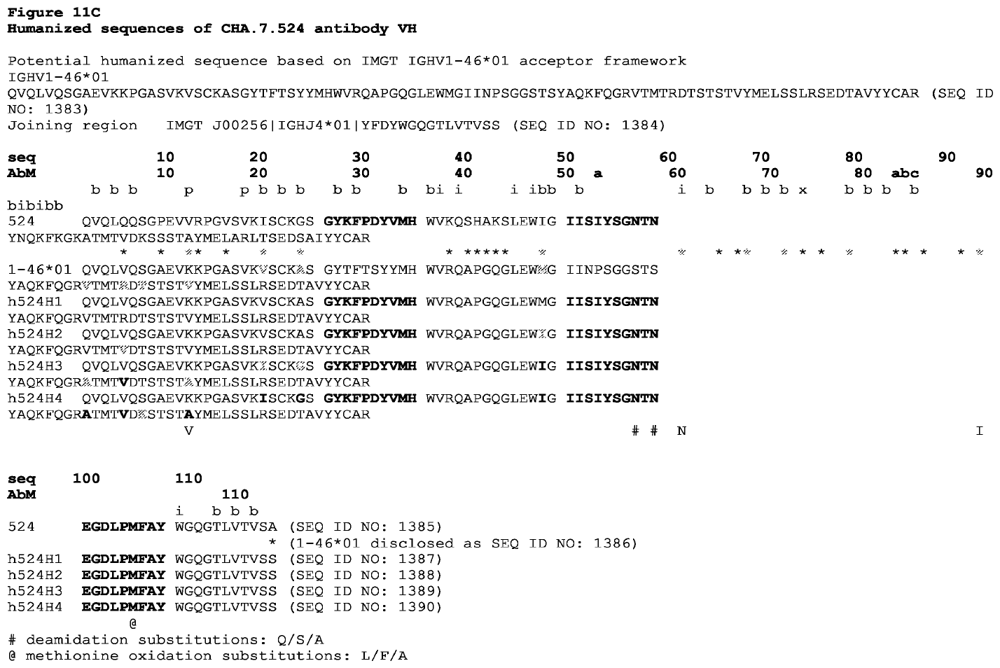

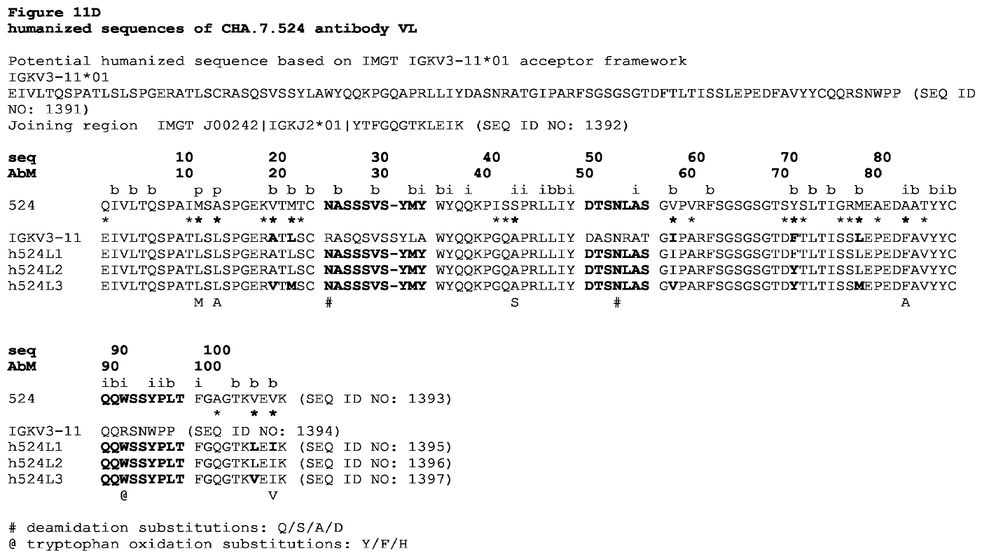

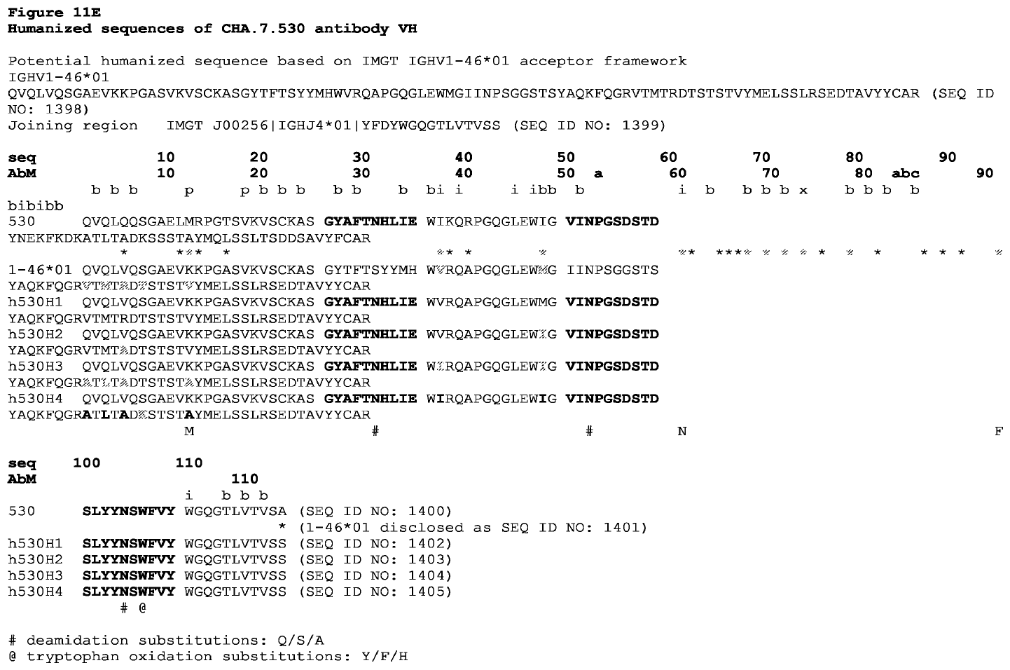

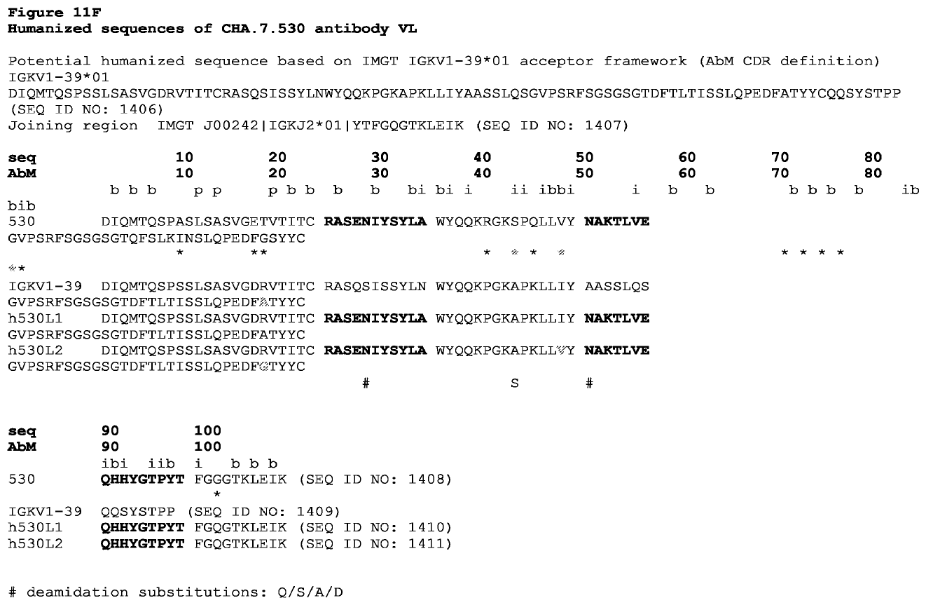

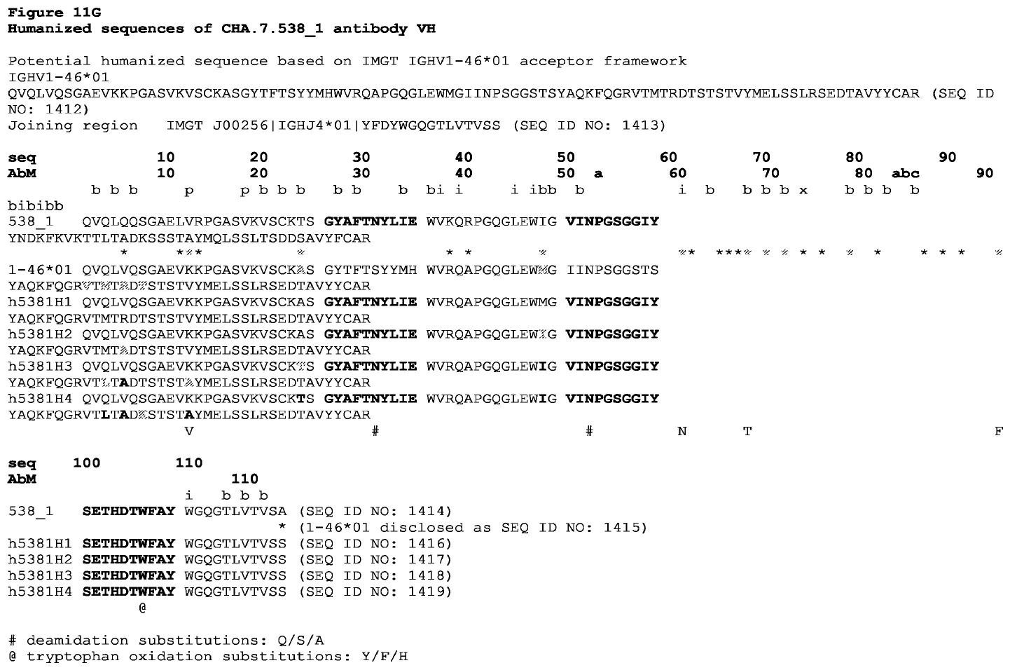

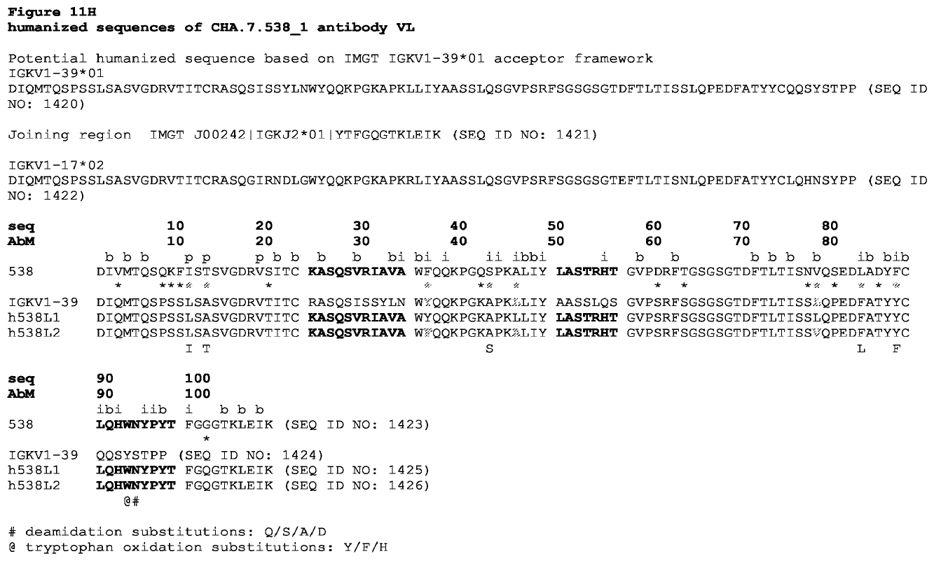

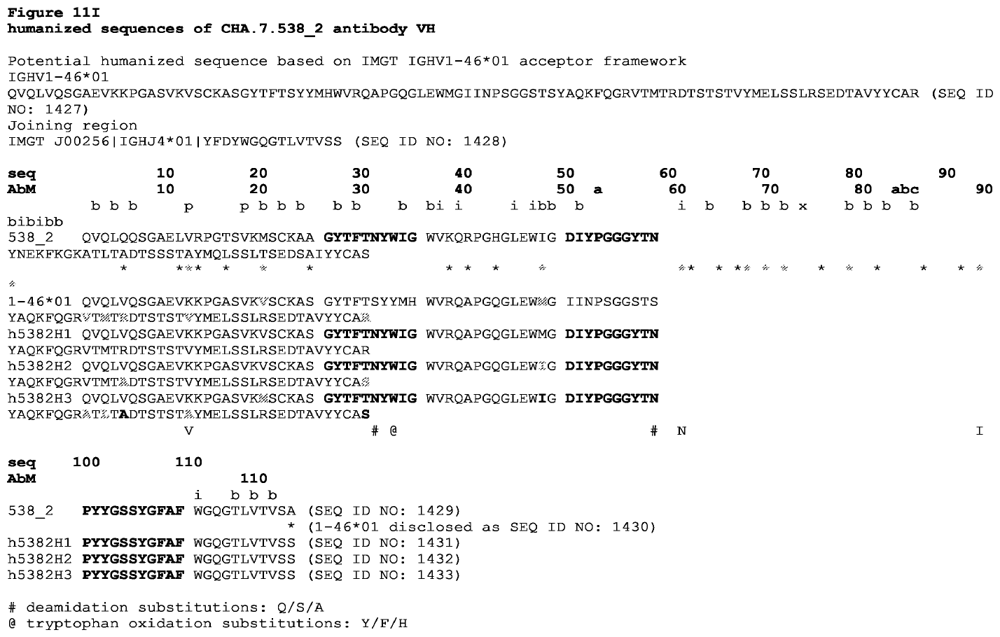

In particular, as is known in the art, murine VH and VL chains can be humanized as is known in the art, for example, using the IgBLAST program of the NCBI website, as outlined in Ye et al. Nucleic Acids Res. 41:W34-W40 (2013), herein incorporated by reference in its entirety for the humanization methods. IgBLAST takes a murine VH and/or VL sequence and compares it to a library of known human germline sequences. As shown herein, for the humanized sequences generated herein, the databases used were IMGT human VH genes (F+ORF, 273 germline sequences) and IMGT human VL kappa genes (F+ORF, 74 germline sequences). An exemplary five CHA sequences were chosen: CHA.7.518, CHA.7.530, CHA.7.538_1, CHA.7.538_2 and CHA.7.524 (see FIGS. 7A-7DD for the V_{H} and V_{L} sequences). For this embodiment of the humanization, human germline IGHV1-46(allele1) was chosen for all 5 as the acceptor sequence and the human heavy chain IGHJ4(allele1) joining region (J gene). For three of four (CHA.7.518, CHA.7.530, CHA.7.538_1 and CHA.7.538_2), human germline IGKV1-39(allele 1) was chosen as the acceptor sequence and human light chain IGKJ2(allele1) (J gene) was chosen. The J gene was chosen from human joining region sequences compiled at IMGT® the international ImMunoGeneTics information system as www.imgt.org. CDRs were defined according to the AbM definition (see www.bioinfo.org.uk/abs/). FIG. 11A-11I depict humanized sequences as well as some potential changes to optimize binding to PVRIG. The anti-PVRIG antibodies for use with the invention can comprise any of these humanized PVRIG antibody or antigen binding domain sequences as the PVRIG binding portion of the anti-PVRIG antibodies. The anti-PVRIG antibodies for use with the invention can comprise CHA.7.518 PVRIG antibody sequences as the PVRIG binding portion of the anti-PVRIG antibodies. The anti-PVRIG antibodies for use with the invention can comprise CHA.7.530 PVRIG antibody sequences as the PVRIG binding portion. The anti-PVRIG antibodies for use with the invention can comprise CHA.7.538_1 PVRIG antibody sequences as the PVRIG binding portion of the anti-PVRIG antibodies. The anti-PVRIG antibodies for use with the invention can comprise CHA.7.538_2 PVRIG antibody sequences as the PVRIG binding portion of the anti-PVRIG antibodies. The anti-PVRIG antibodies for use with the invention can comprise CHA.7.518.4 PVRIG antibody sequences as the PVRIG binding portion of the anti-PVRIG antibodies

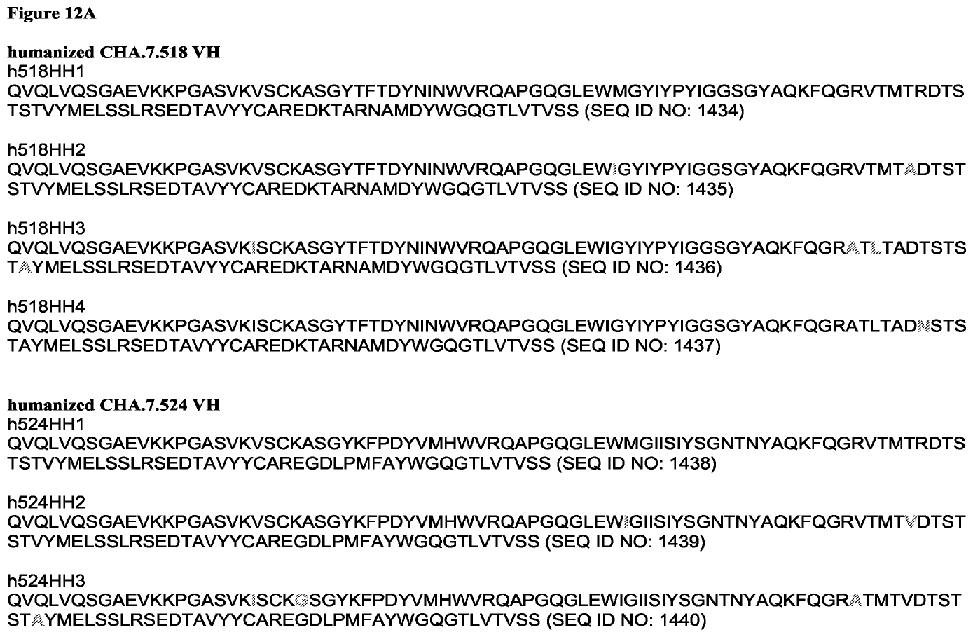

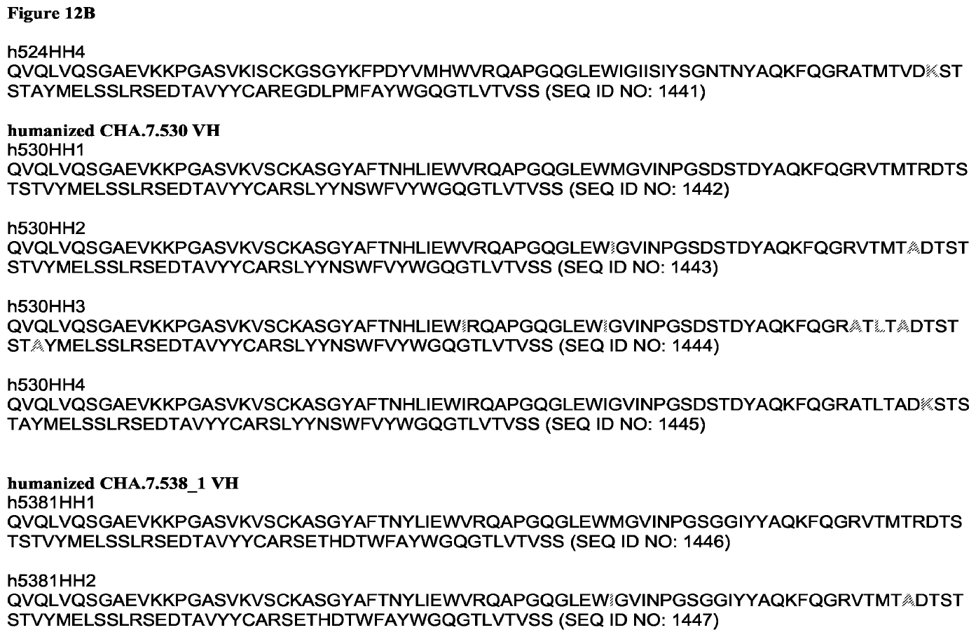

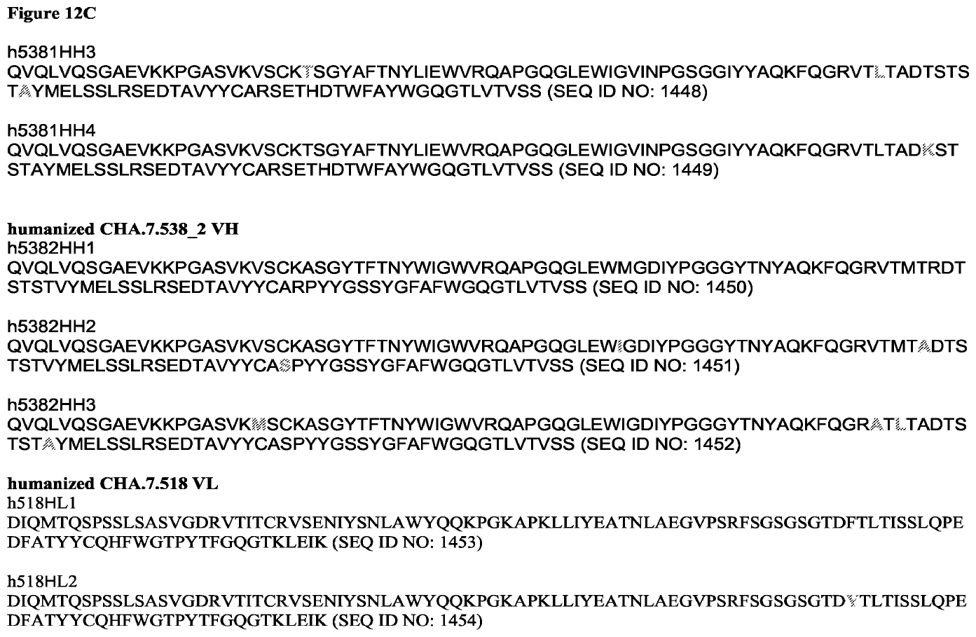

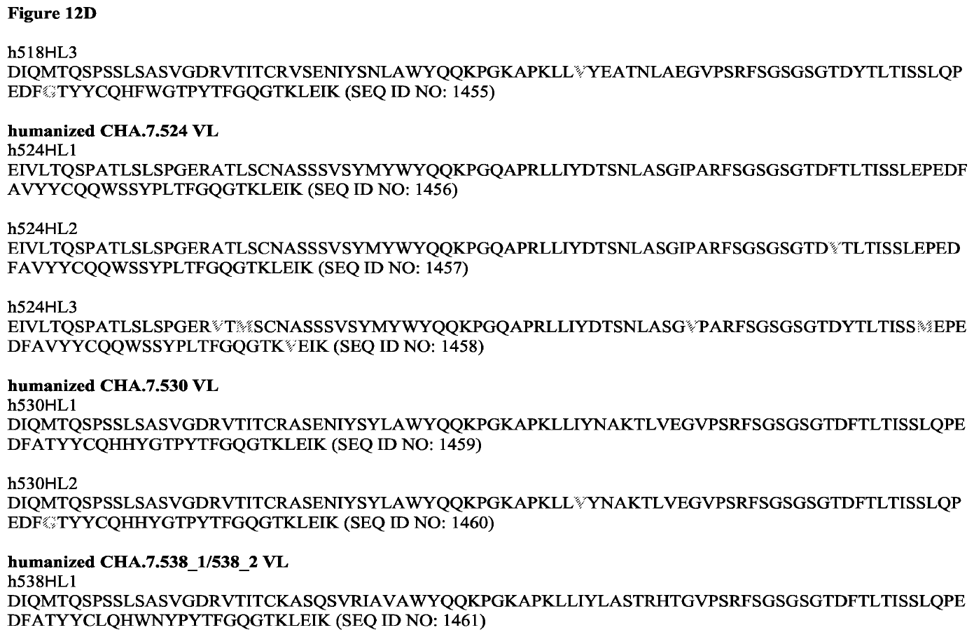

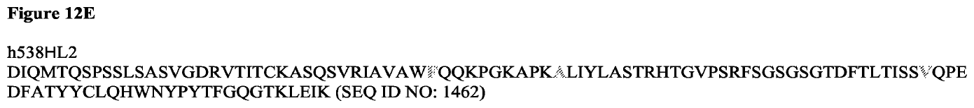

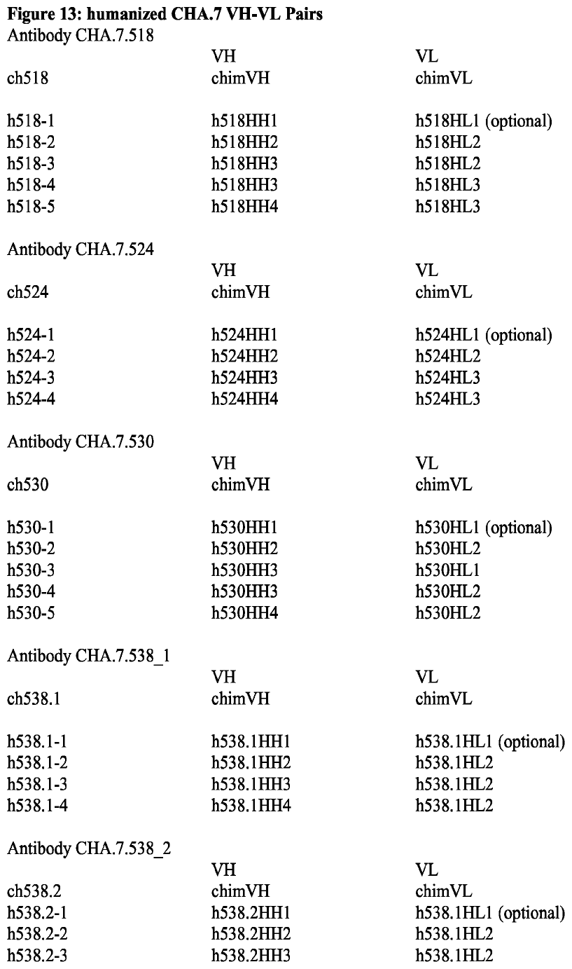

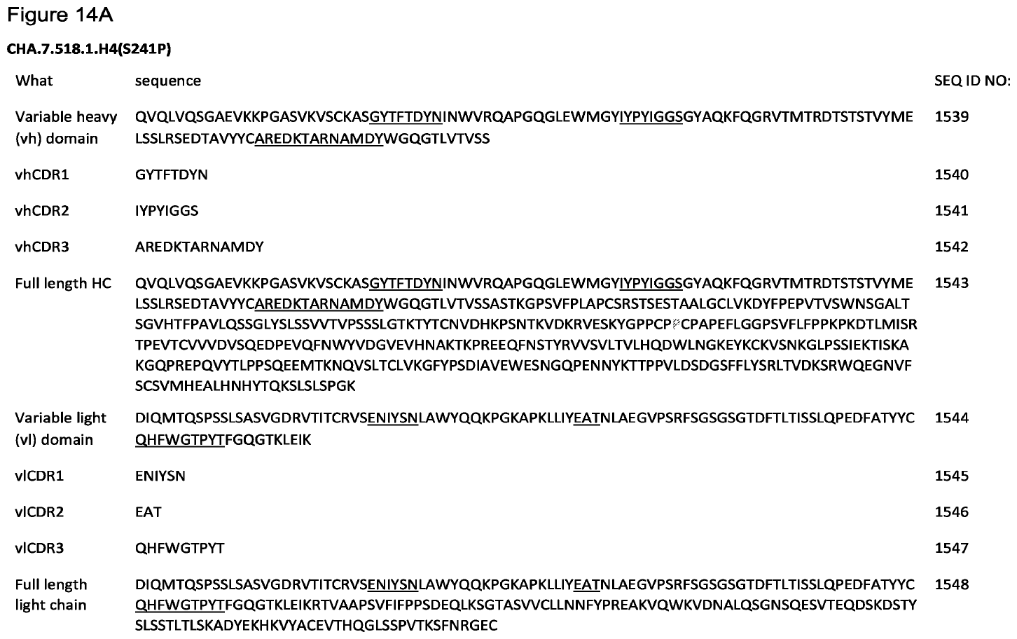

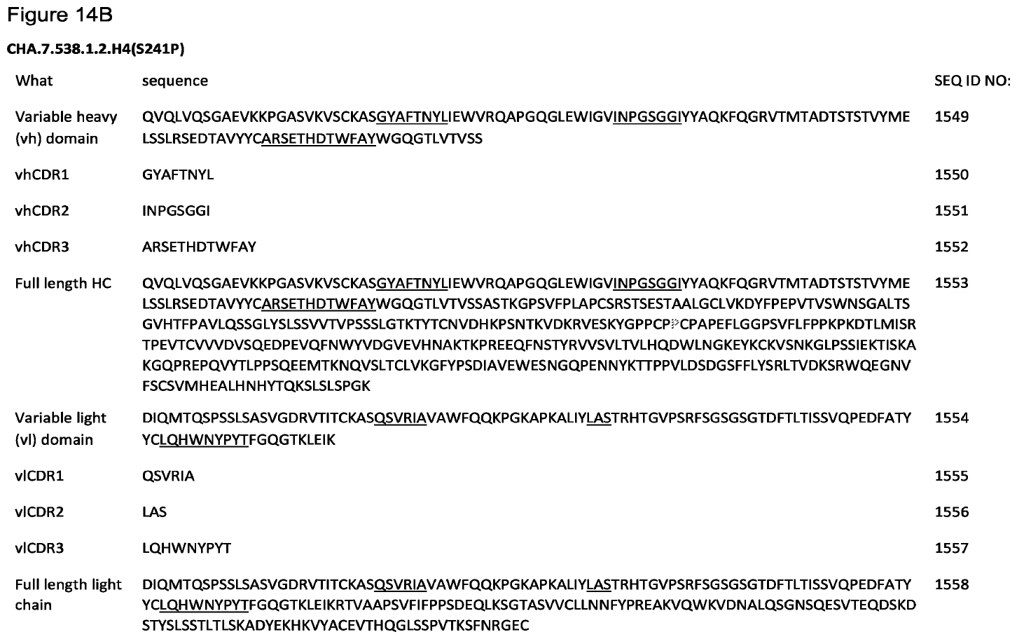

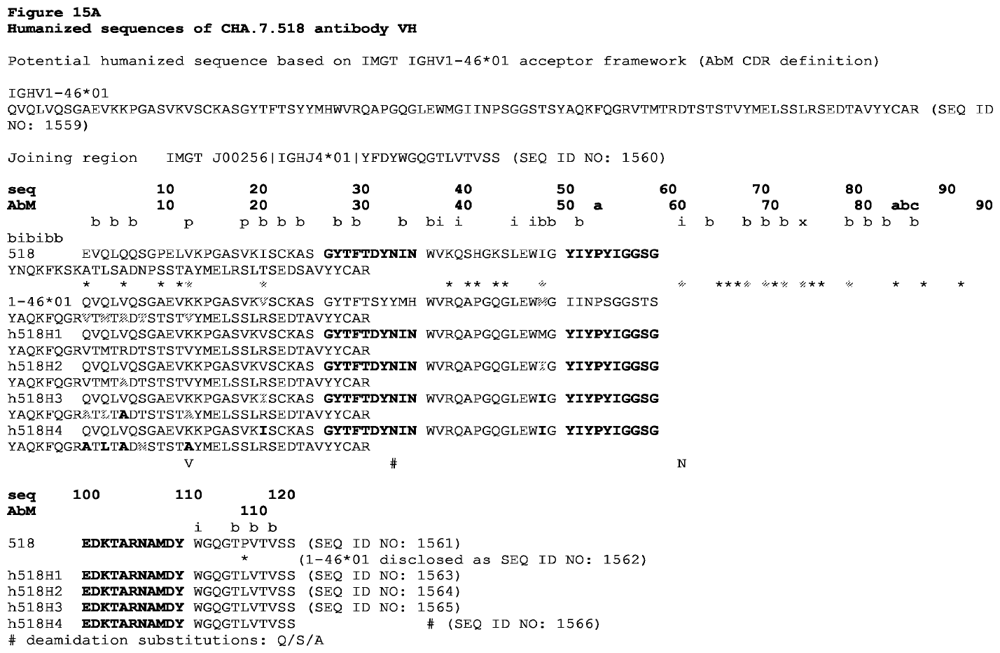

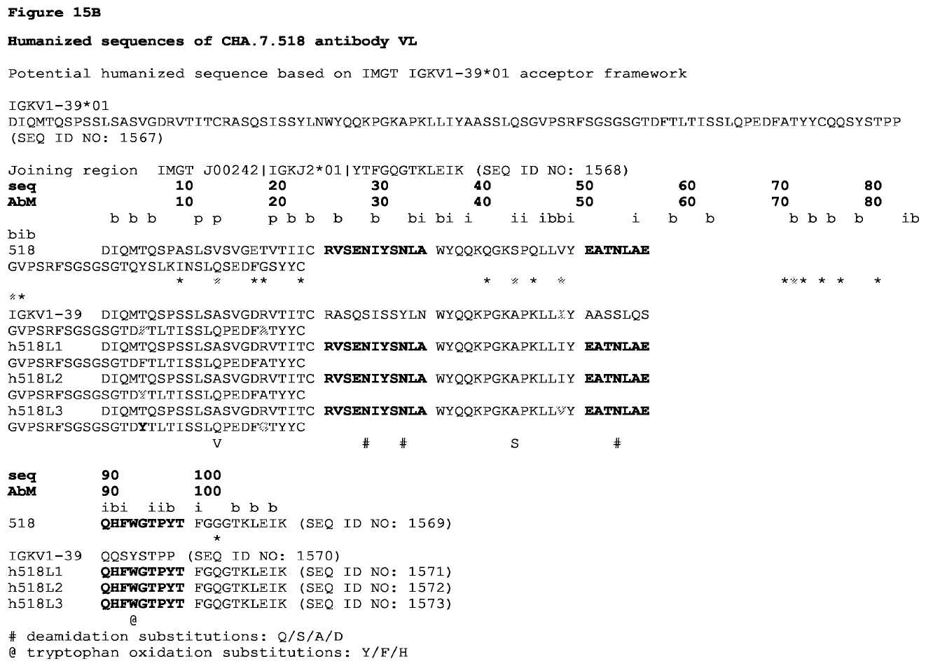

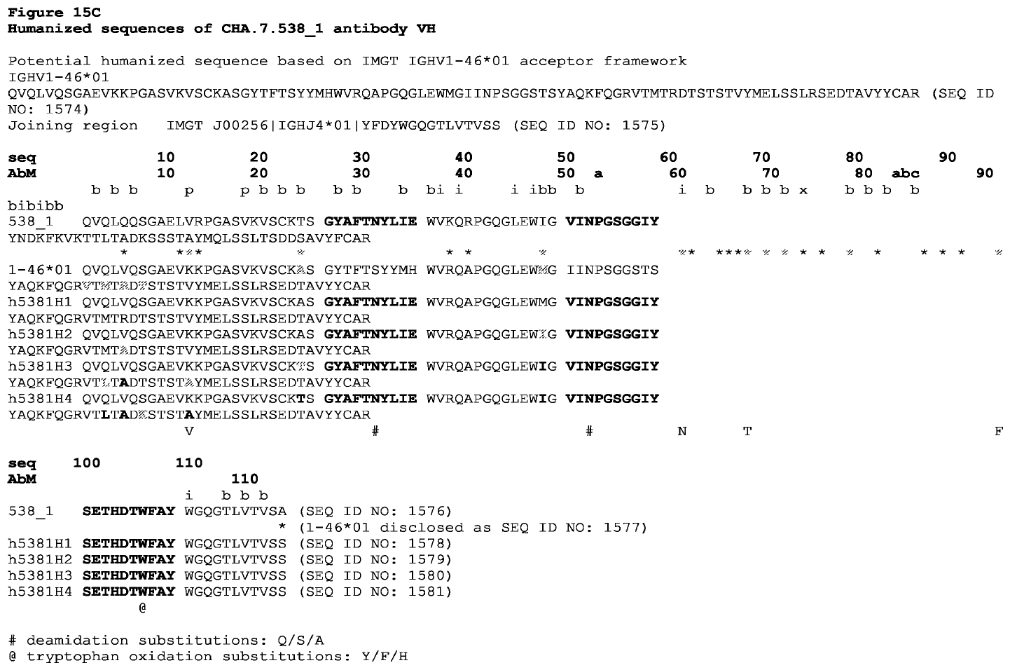

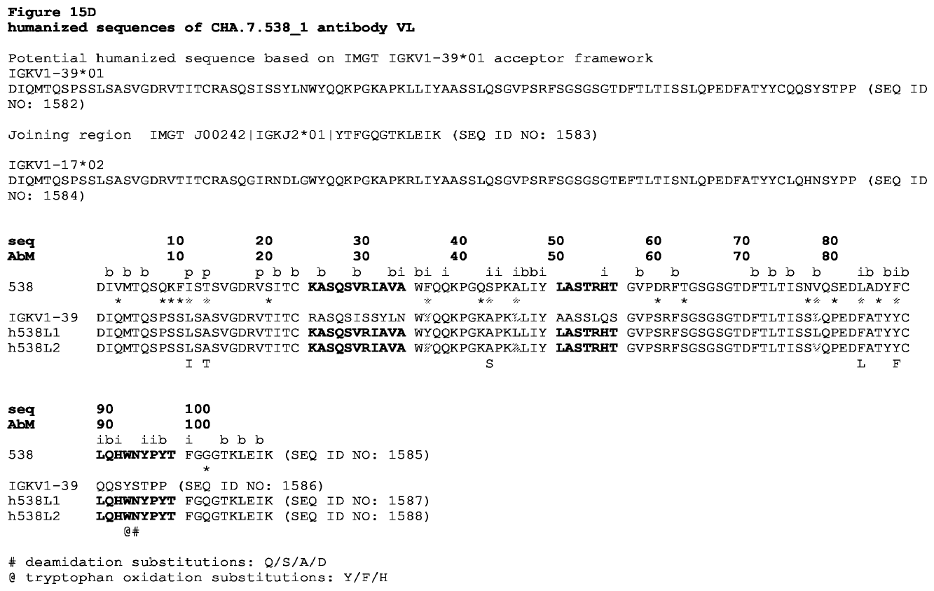

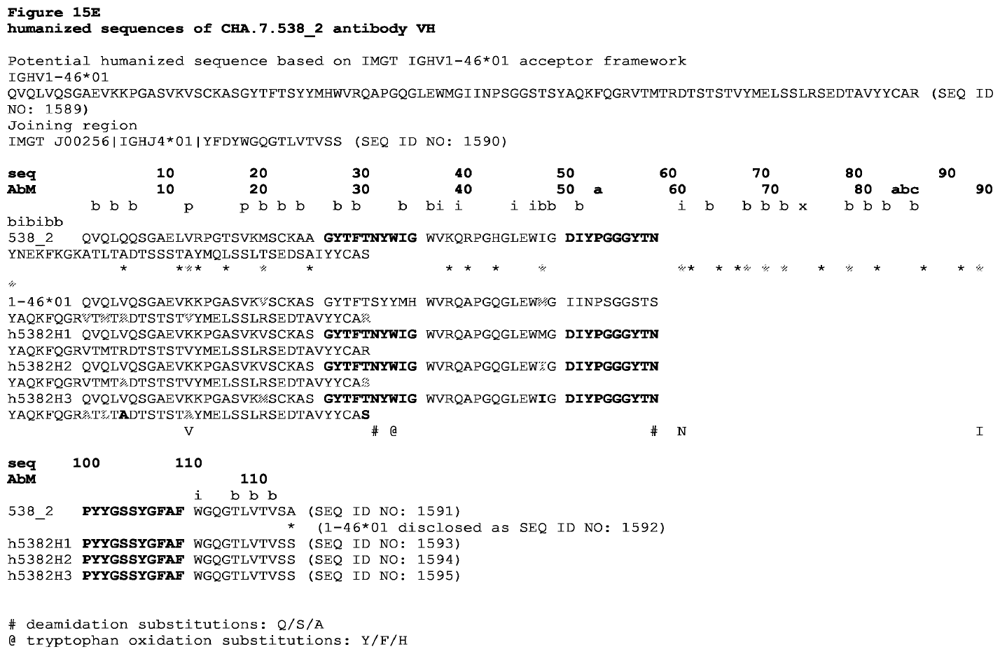

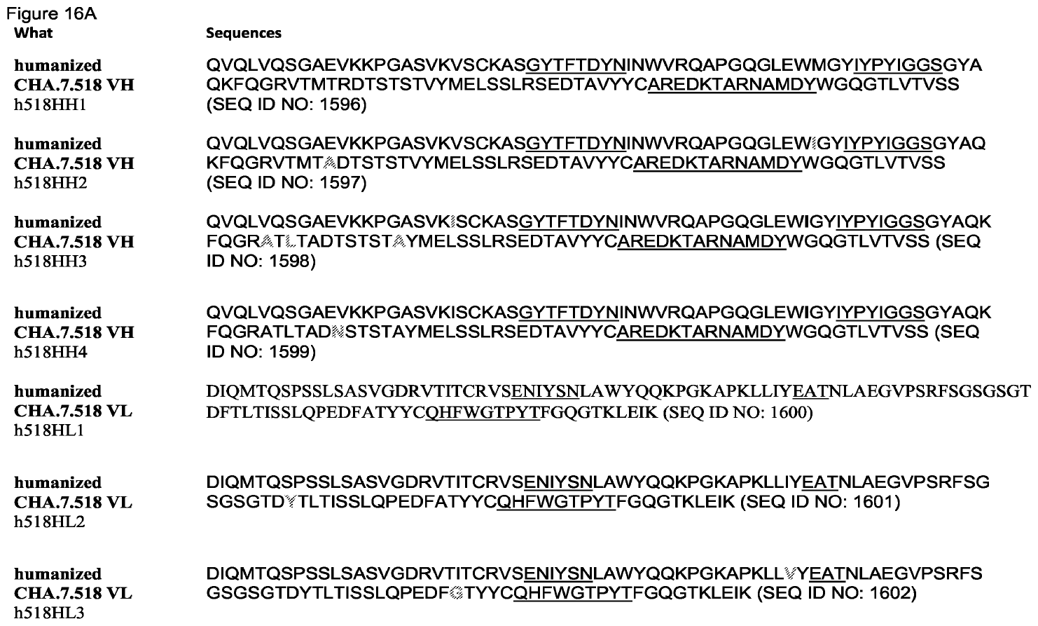

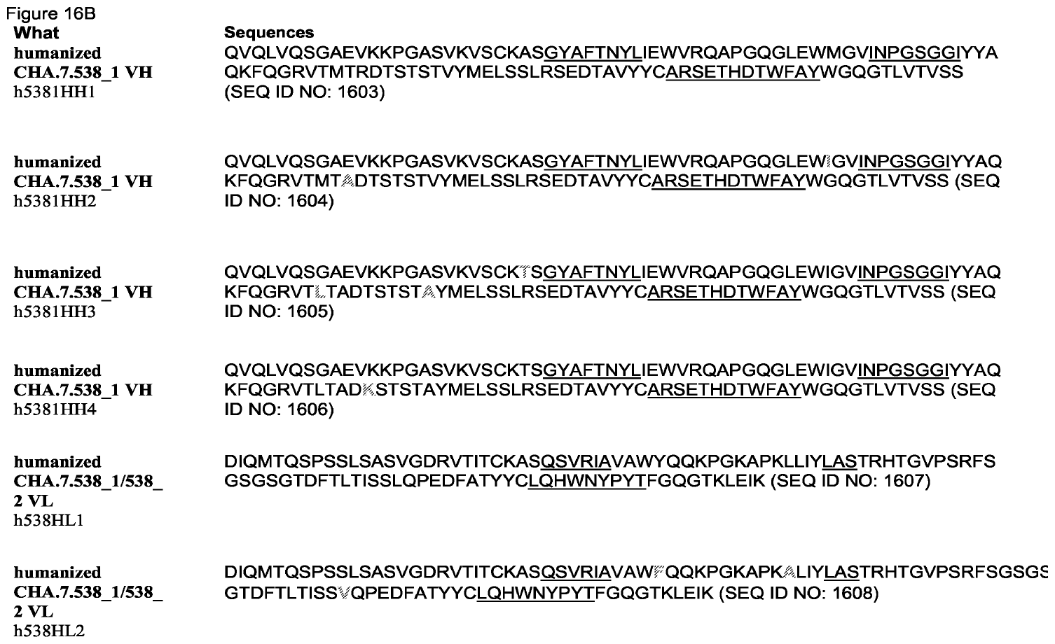

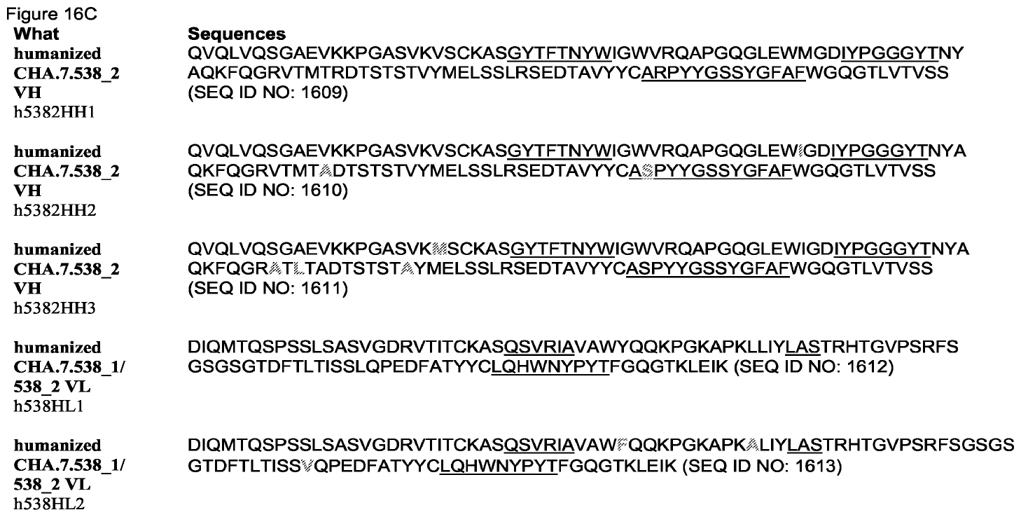

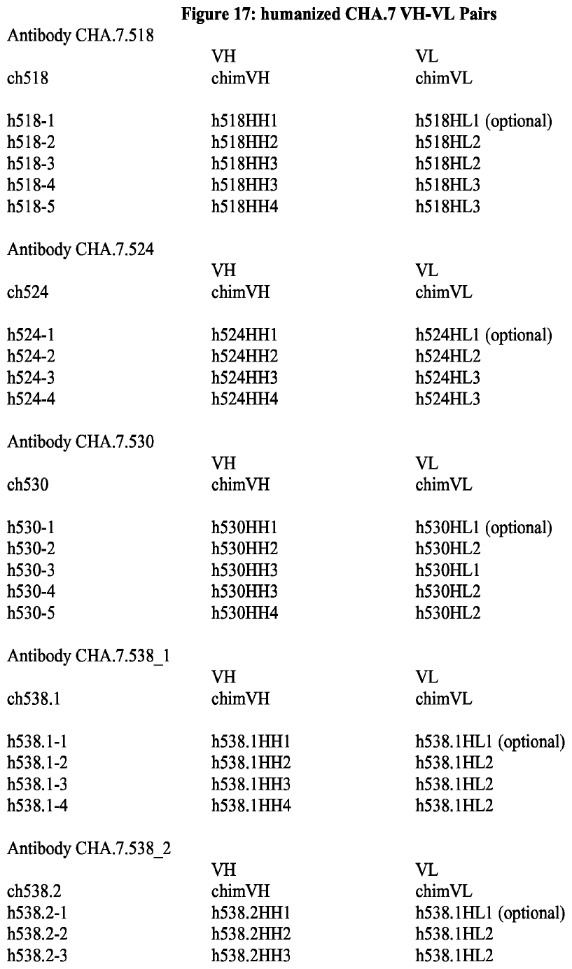

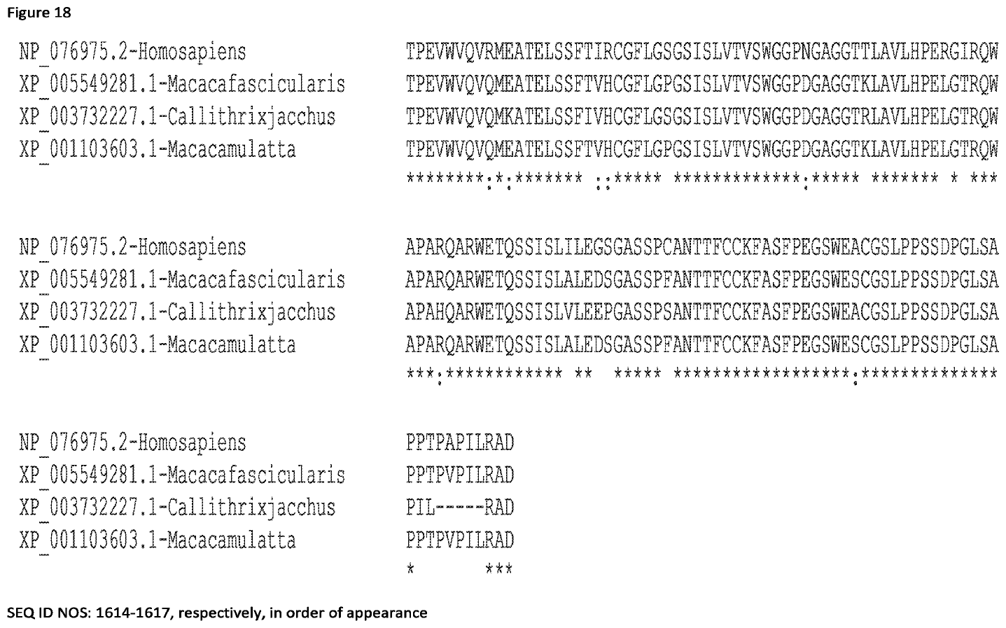

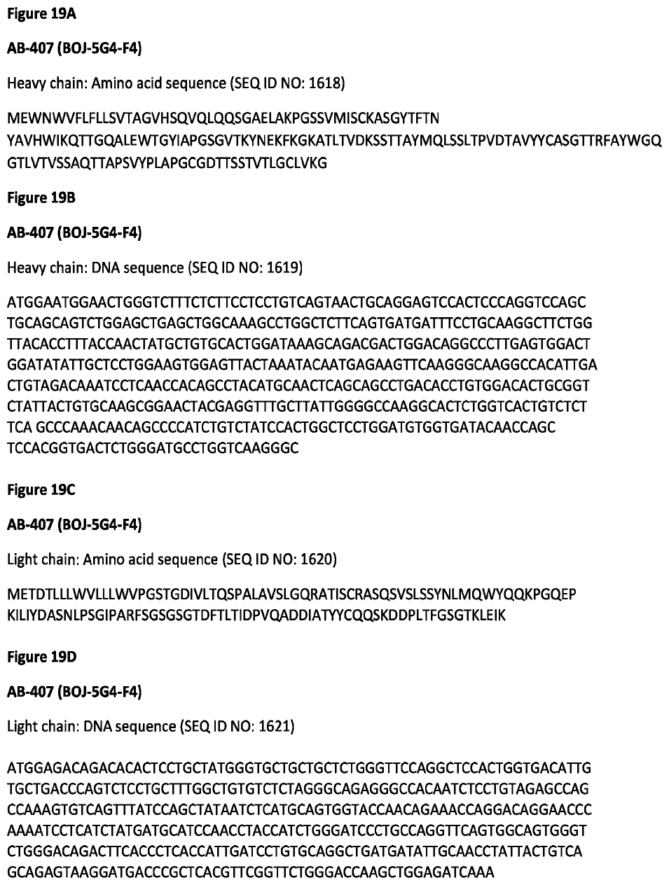

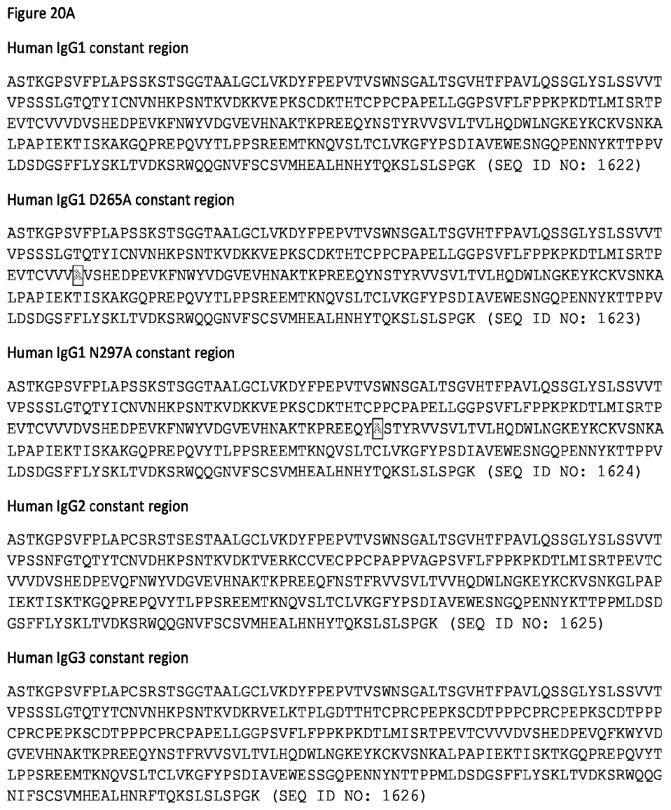

Specific humanized antibodies of CHA antibodies include those shown in FIGS. 11A-11I, FIGS. 12A-12E and FIG. 13. The anti-PVRIG antibodies for use with the invention can comprise CHA PVRIG antibody sequences as shown in in FIGS. 11A-11I, FIGS. 12A-12E and FIG. 13 as the PVRIG binding portion of the anti-PVRIG antibodies. As will be appreciated by those in the art, each humanized variable heavy (Humanized Heavy; HH) and variable light (Humanized Light, HL) sequence can be combined with the constant regions of human IgG1, IgG2, IgG3 and IgG4. That is, CHA.7.518.HH1 is the first humanized variable heavy chain, and CHA.7.518.HH1.1 is the full length heavy chain, comprising the “HH1” humanized sequence with a IgG1 constant region (CHA.7.518.HH1.2 is CHA.7.518.HH1 with IgG2, etc.). In some embodiments, anti-PVRIG antibody comprises the PVRIG sequences provided in FIGS. 4A-4AA, 5A-5H, 7A-7AE, 11A-11I, 12A-12E, 13, 14A-14BX, 15A-15B, and 16A-16E, and 17A-17C, as the PVRIG binding portion.

In some embodiments, the anti-PVRIG antibodies of the present invention include anti-PVRIG antibodies wherein the V_{H} and V_{L} sequences of different anti-PVRIG antibodies can be “mixed and matched” to create other anti-PVRIG antibodies. PVRIG binding of such “mixed and matched” antibodies can be tested using the binding assays described above. e.g., ELISAs). In some embodiments, when V_{H} and V_{L} chains are mixed and matched, a V_{H} sequence from a particular V_{H}/V_{L} pairing is replaced with a structurally similar V_{H} sequence. Likewise, in some embodiments, a V_{L} sequence from a particular V_{H}/V_{L} pairing is replaced with a structurally similar V_{L} sequence. For example, the V_{H} and V_{L} sequences of homologous antibodies are particularly amenable for mixing and matching. The anti-PVRIG antibodies for use with the invention can comprise PVRIG V_{H} and V_{L} sequences from different anti-PVRIG antibodies that have been “mixed and matched” as the PVRIG binding portion.

Accordingly, the antibodies for use with the invention comprise CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more amino acid substitutions; (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein. The anti-PVRIG antibodies for use with the invention can comprise PVRIG variant CDR sequences as part of the PVRIG binding portion.

Additionally included in the definition of PVRIG antibodies are antibodies that share identity to the PVRIG antibodies enumerated herein. That is, in certain embodiments, an anti-PVRIG antibody according to the invention comprises heavy and light chain variable regions comprising amino acid sequences that are homologous to isolated anti-PVRIG amino acid sequences of preferred anti-PVRIG immune molecules, respectively, wherein the antibodies retain the desired functional properties of the parent anti-PVRIG antibodies. The percent identity between the two sequences is a function of the number of identical positions shared by the sequences (e.g., % homology=# of identical positions/total # of positions X 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below. The anti-PVRIG antibodies for use with the invention can comprise heavy and light chain variable regions comprising amino acid sequences that are homologous to isolated anti-PVRIG amino acid sequences as described herein.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between PVRIG antibodies is at least 75%, at least 80%, at least 90%, with at least about 95%, 96%, 97%, 98%, or 99% percent identity being preferred. The percentage identity may be along the whole amino acid sequence, for example the entire heavy or light chain or along a portion of the chains. For example, included within the definition of the anti-PVRIG antibodies for use with the invention are those that share identity along the entire variable region (for example, where the identity is 95% or 98% identical along the variable regions), or along the entire constant region, or along just the Fc domain. In particular, the invention provides anti-PVRIG antibodies that have PVRIG binding portions or antigen binding domains with at least 75%, at least 80%, at least 90%, with at least about 95%, 96%, 97%, 98%, or 99% percent identity being preferred, with the CHA.7.518.4 antibody.

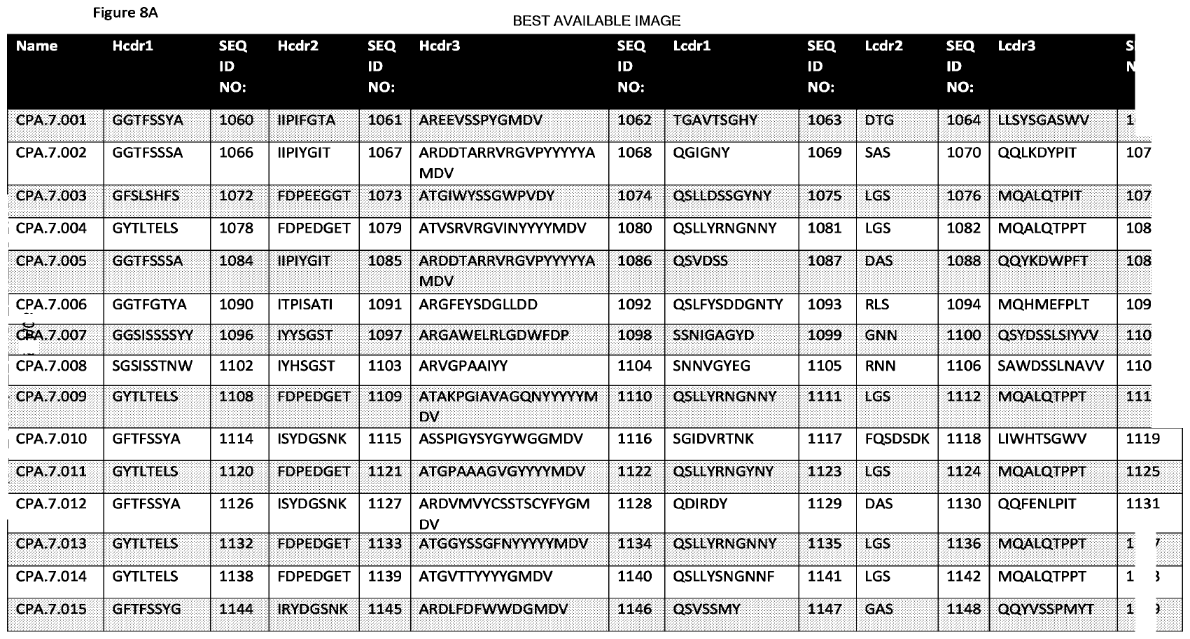

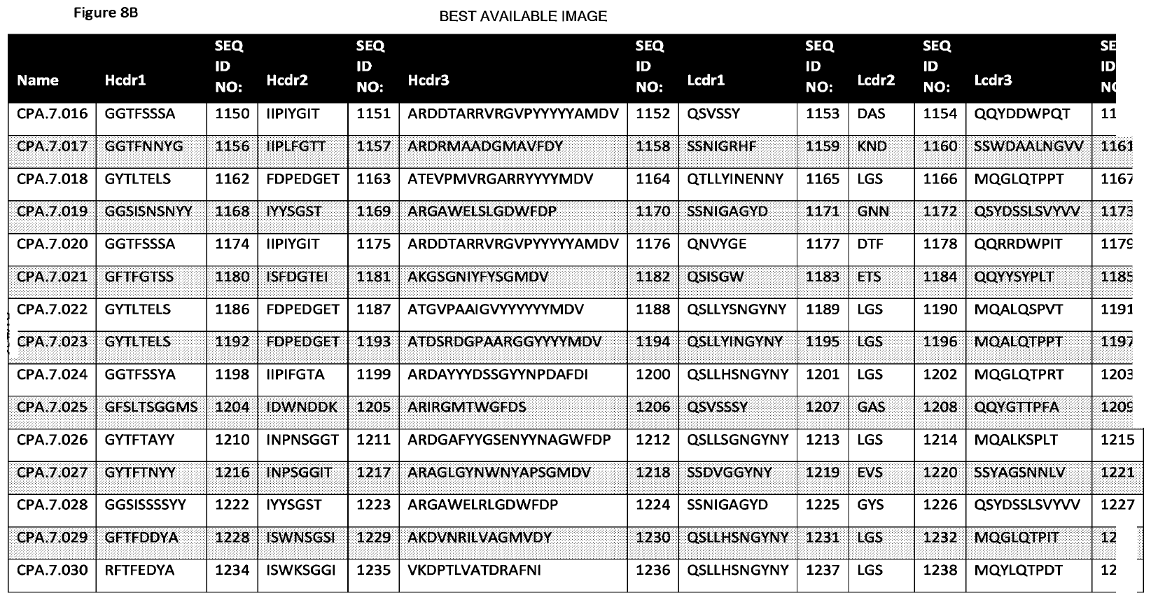

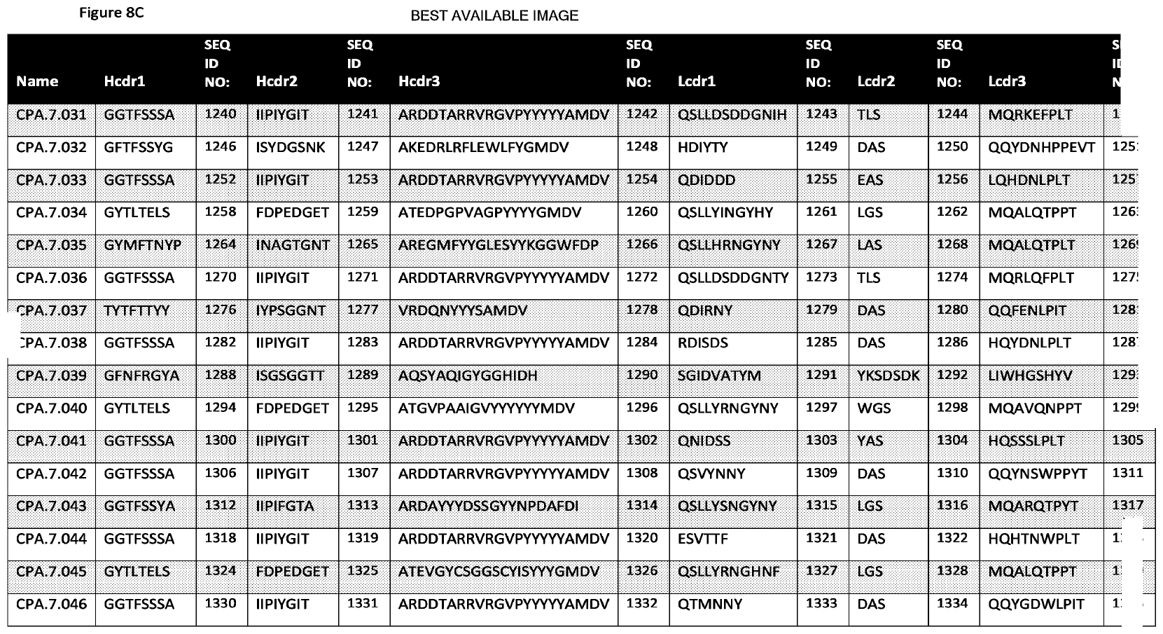

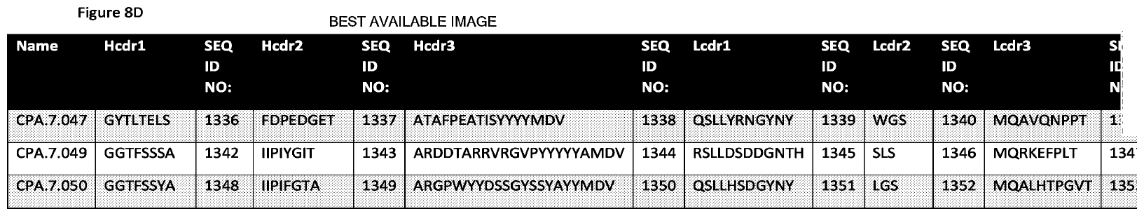

In addition, also included are sequences that may have the identical CDRs but changes in the variable domain (or entire heavy or light chain). For example, PVRIG antibodies include those with CDRs identical to those shown in FIGS. 8A-8D but whose identity along the variable region can be lower, for example 95 or 98% percent identical. In particular, the invention provides anti-PVRIG antibodies that have PVRIG binding portions or antigen binding domains with identical CDRs to CHA.7.518.4 but with framework regions that are 95% or 98% identical to CHA.7.518.4.

The anti-PVRIG antibodies for use with the invention can comprise CDRs identical to those shown in FIGS. 8A-8D as part of the PVRIG binding portion.

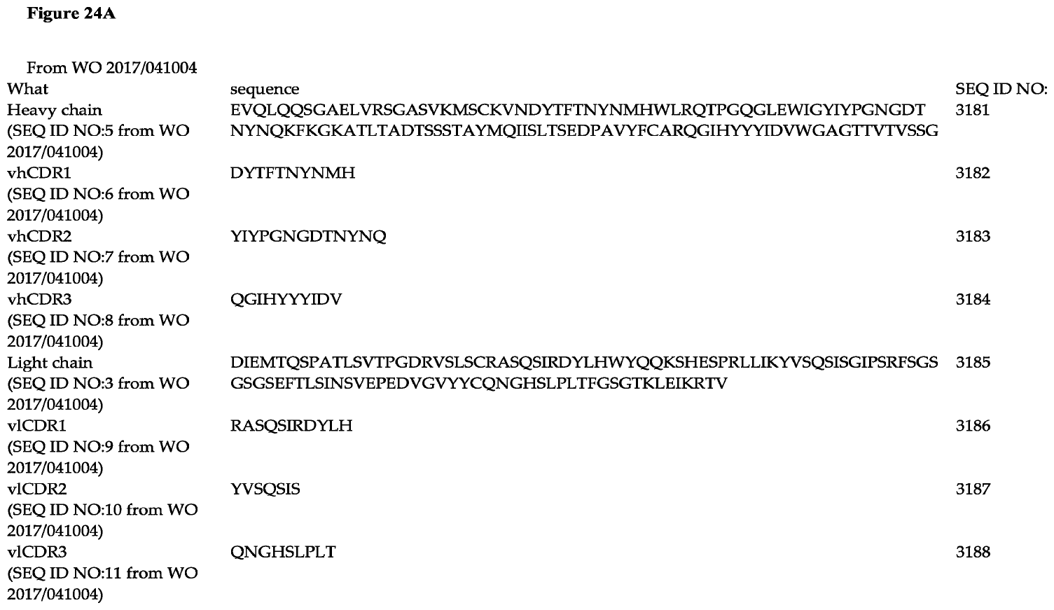

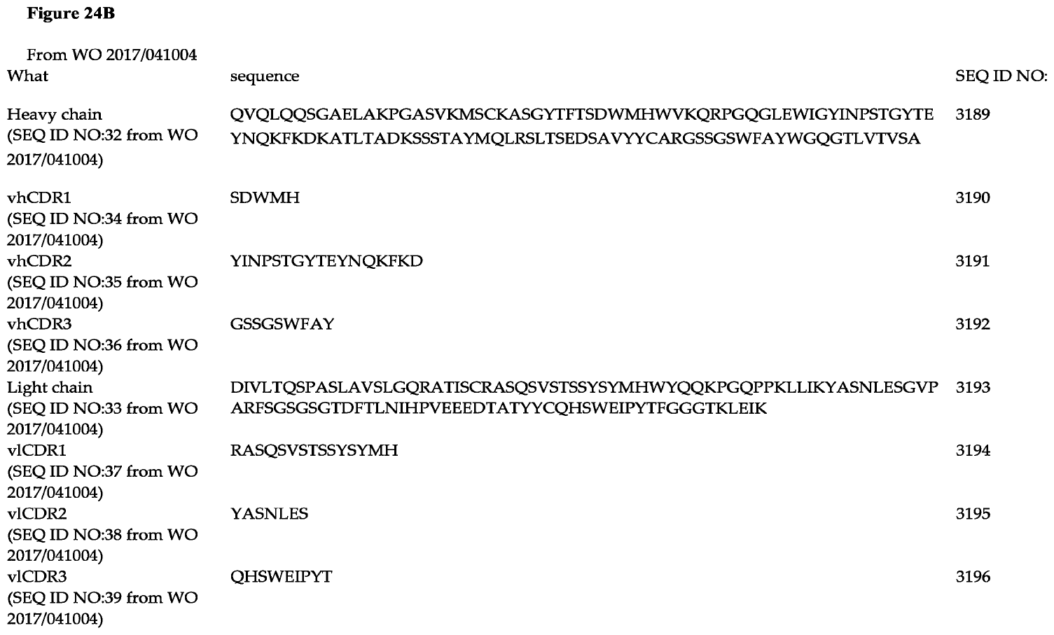

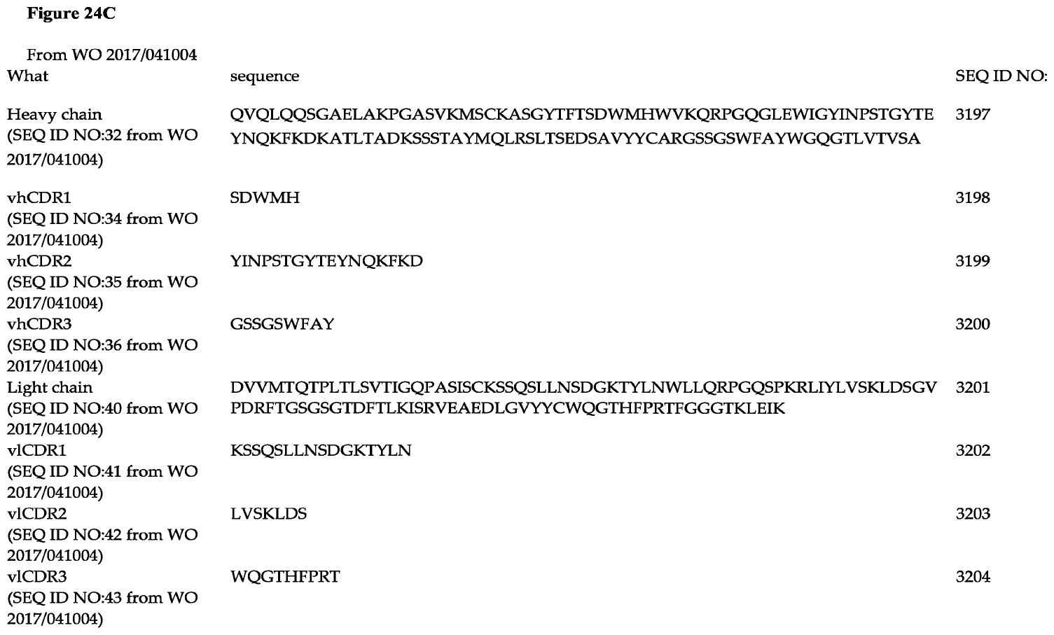

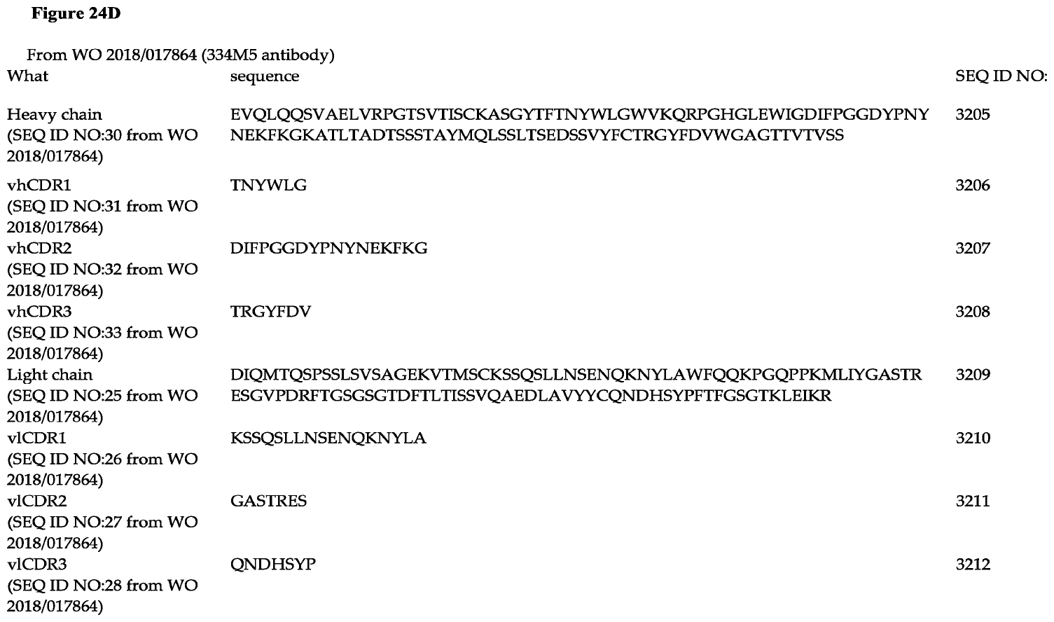

In addition, also included are sequences that may have the identical CDRs but changes in the variable domain (or entire heavy or light chain). For example, PVRIG antibodies include those with CDRs identical to those shown in FIGS. 24A-24D but whose identity along the variable region can be lower, for example 95 or 98% percent identical.

The anti-PVRIG antibodies for use with the invention can comprise CDRs identical to those shown in FIGS. 24A-24D as part of the PVRIG binding portion.

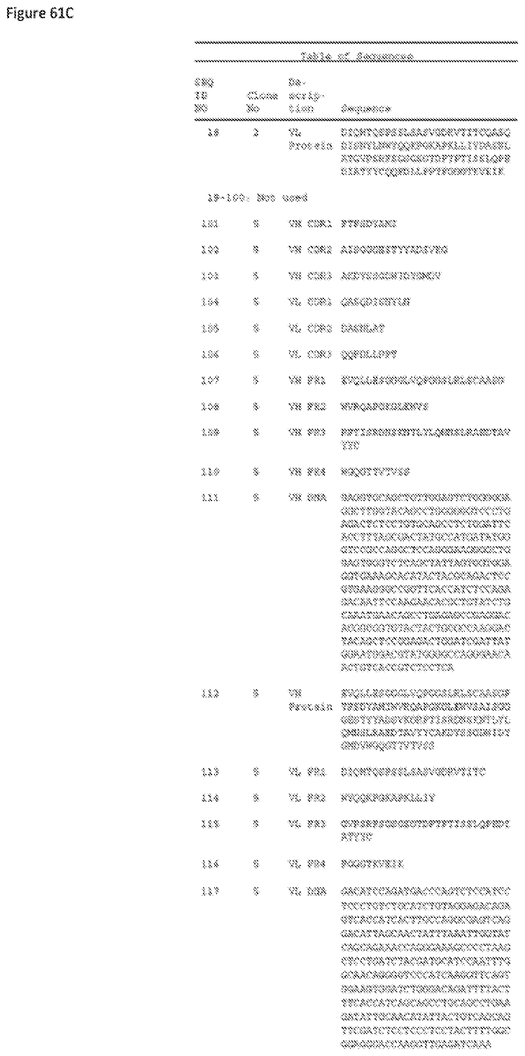

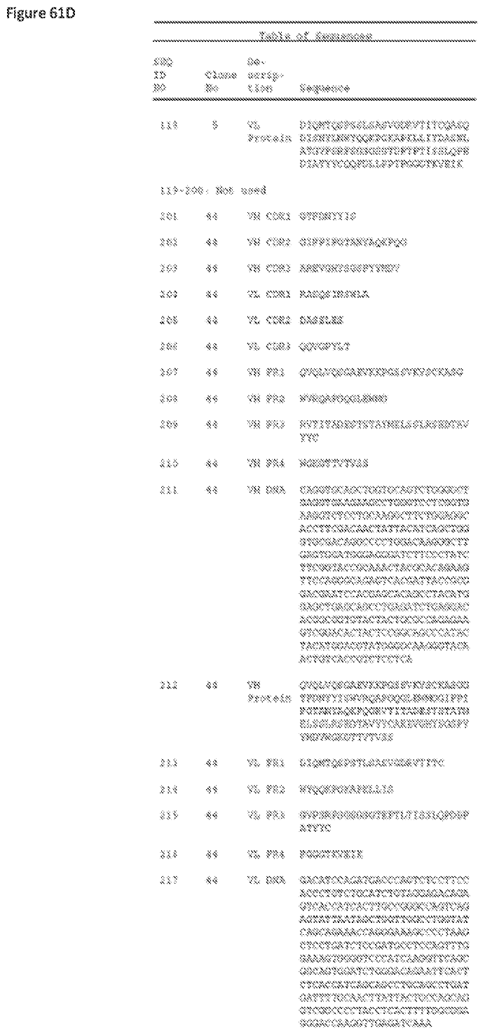

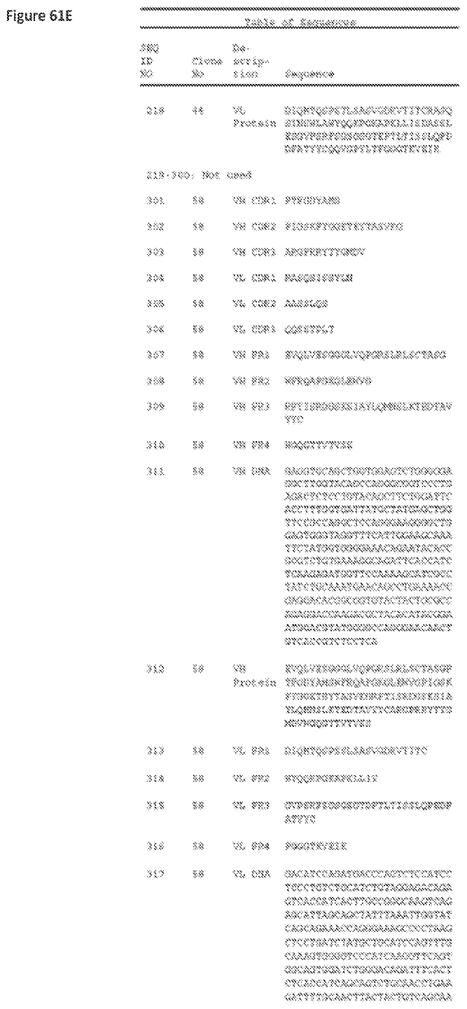

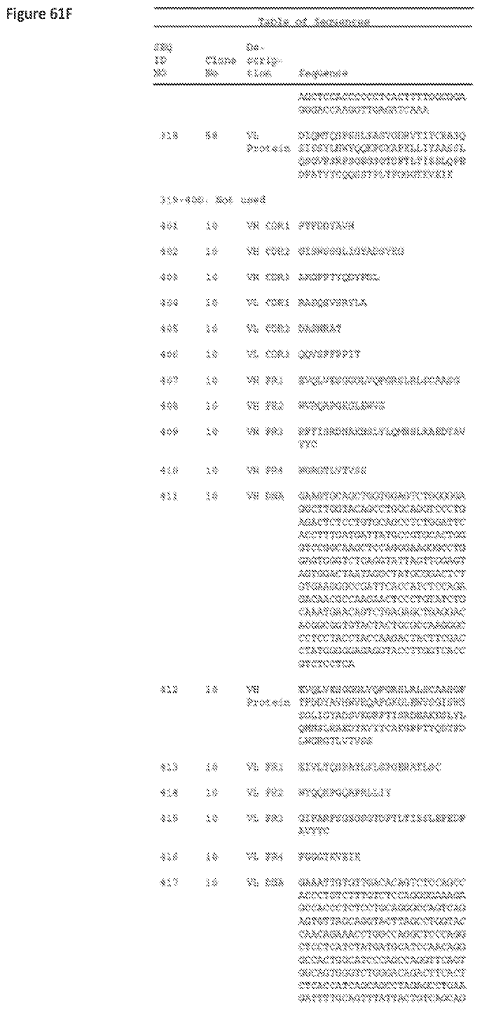

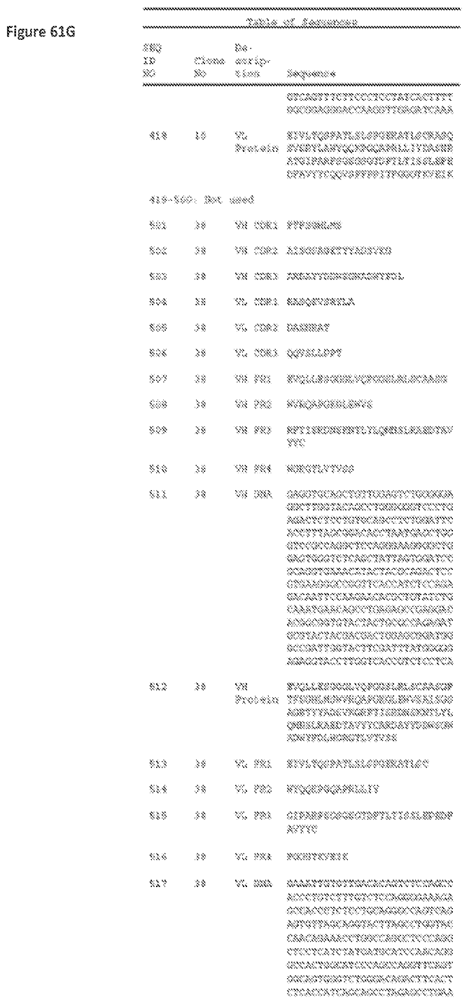

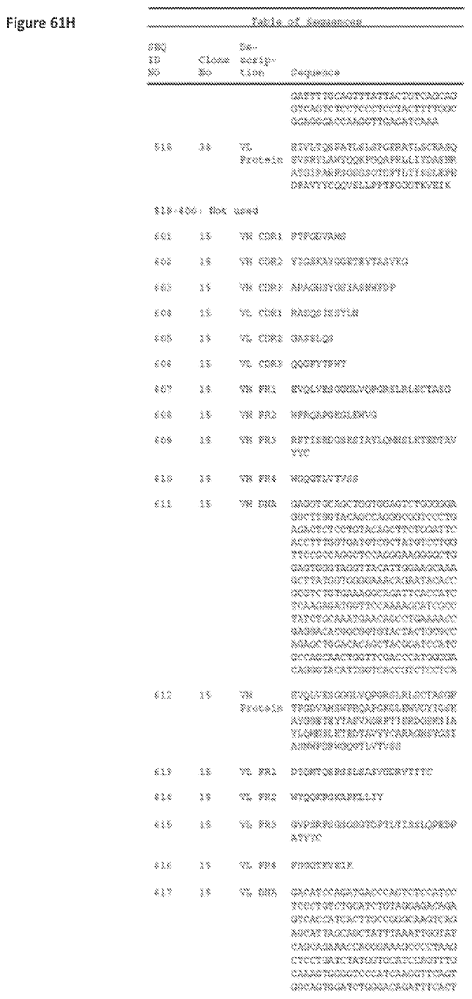

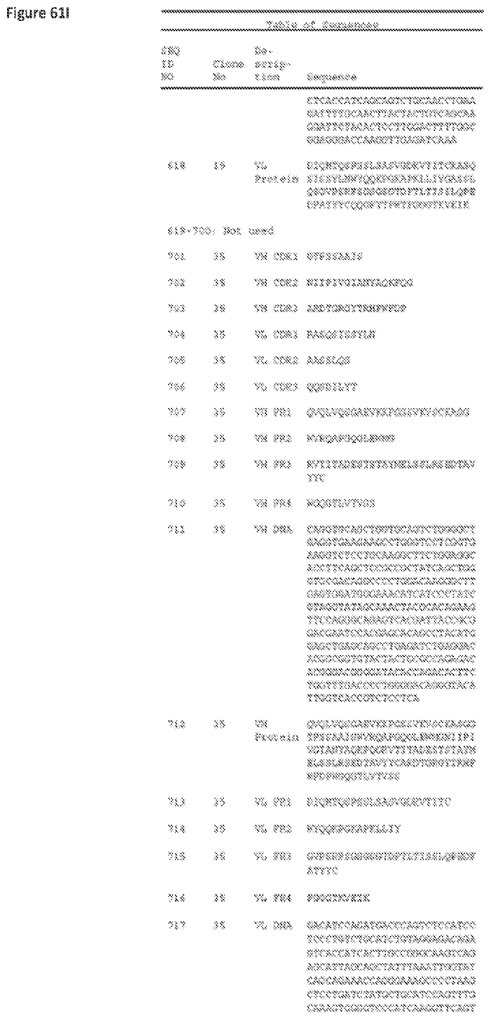

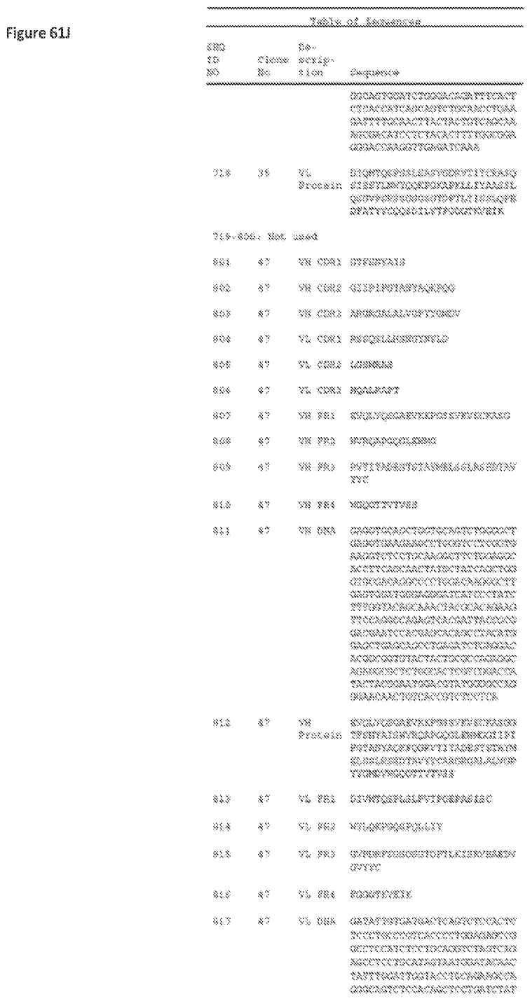

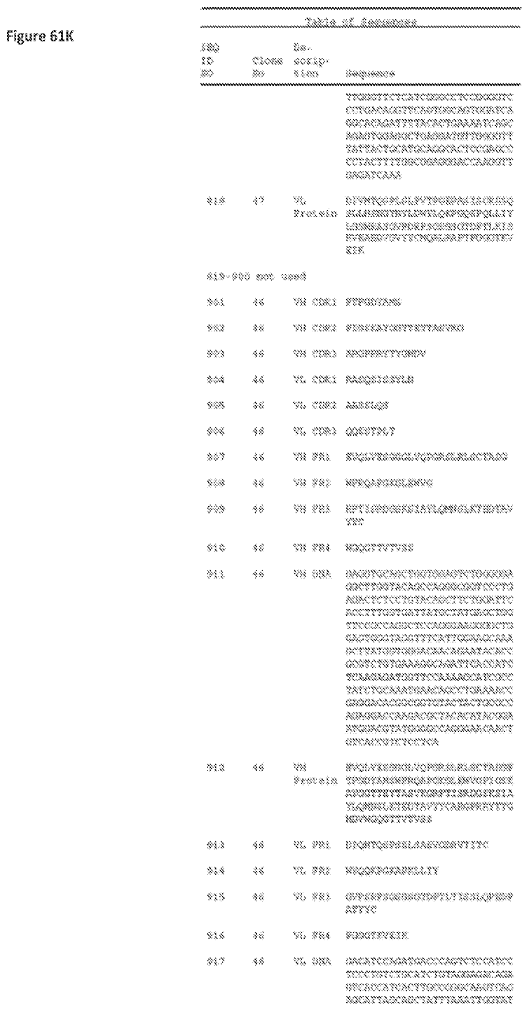

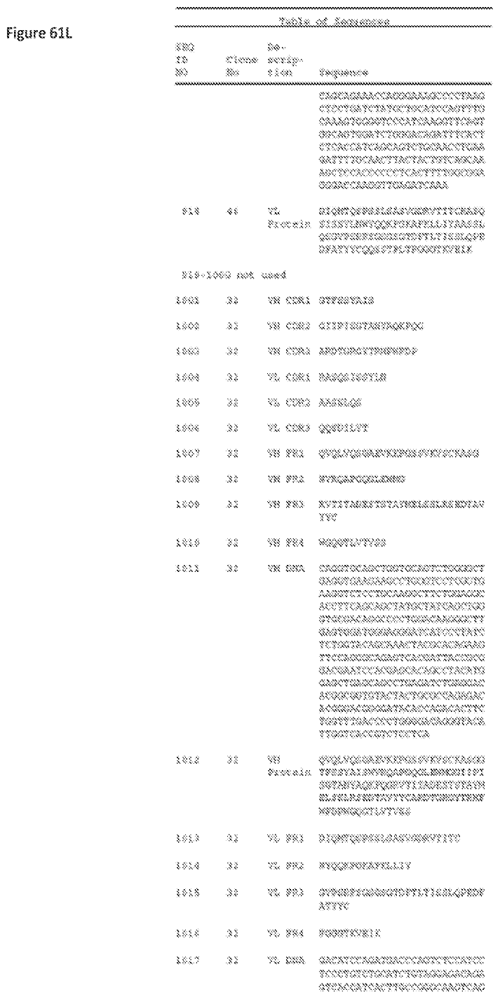

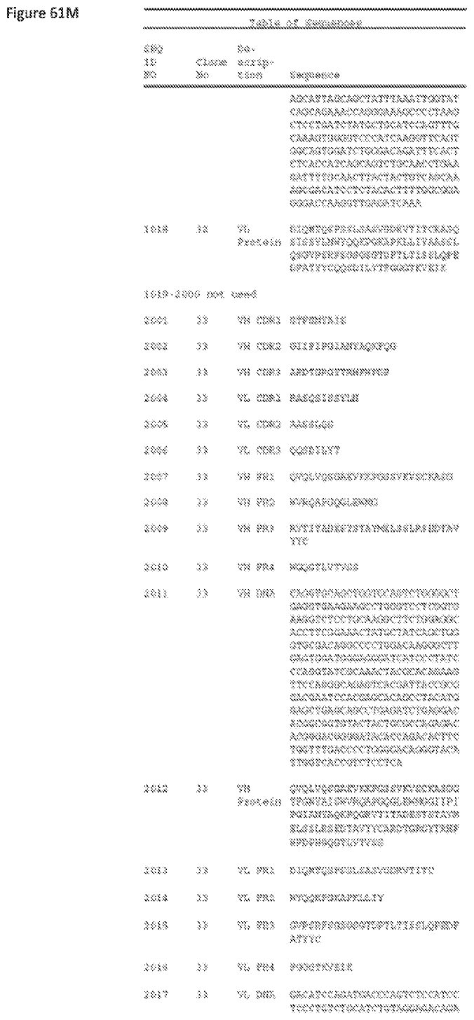

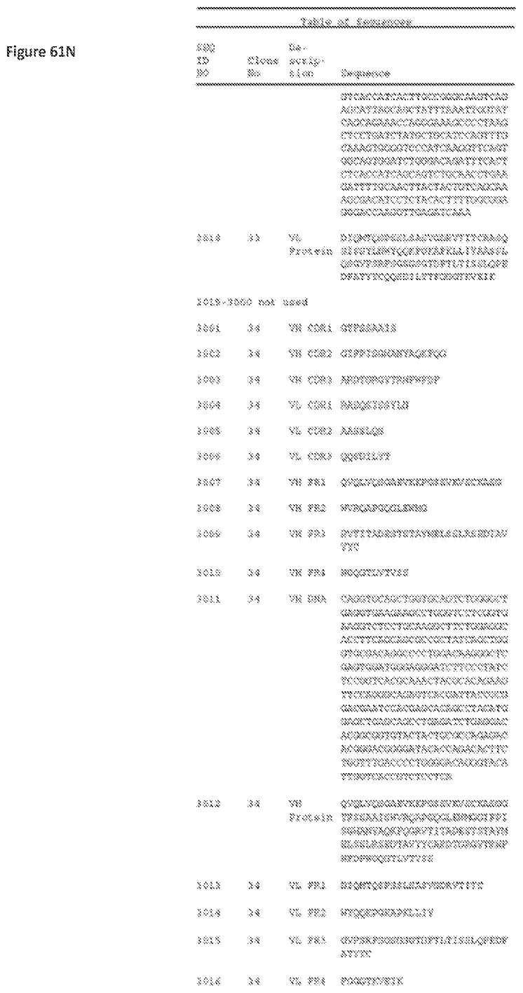

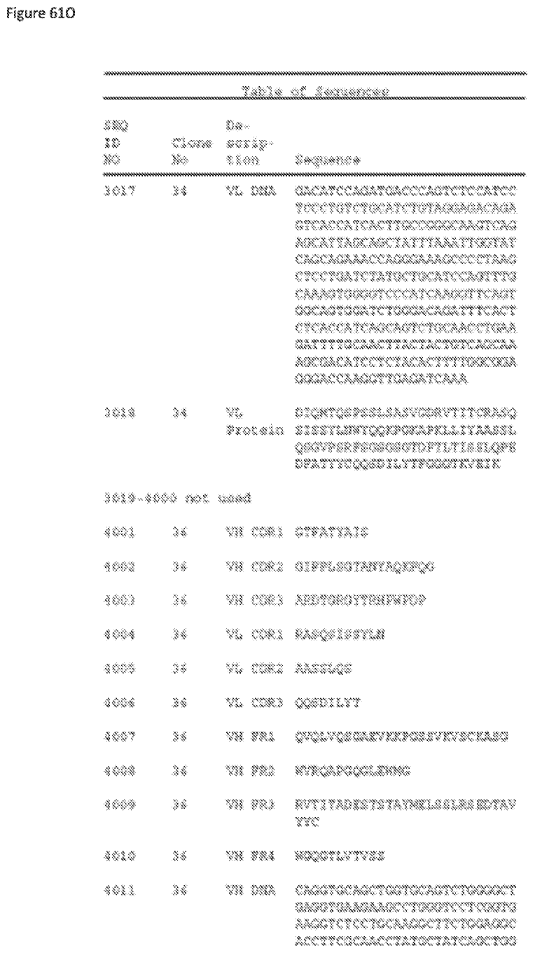

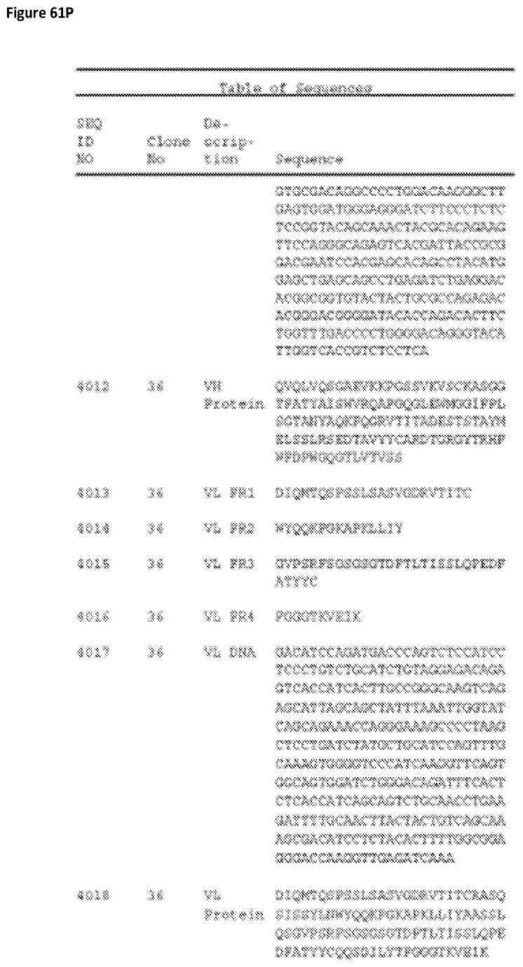

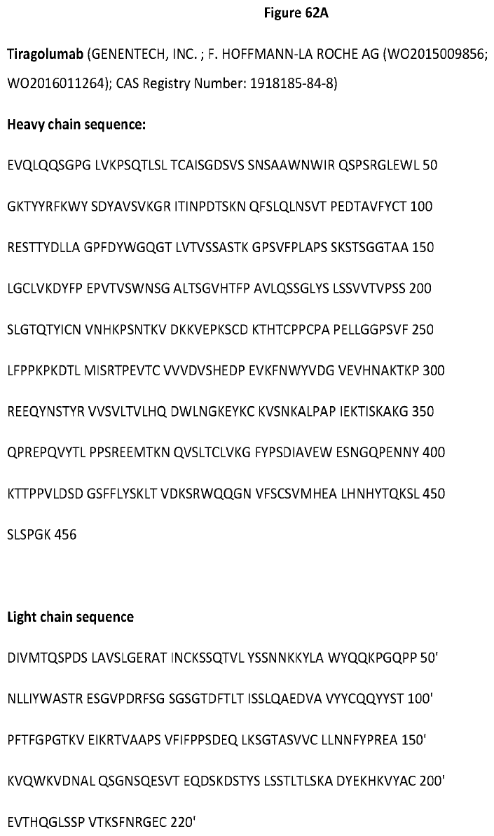

In some embodiments, the anti-PVRIG antibodies for use with the invention can comprise CDRs identical to those shown in FIGS. 61A-61P as part of the PVRIG binding portion. In some embodiments, the anti-PVRIG antibodies for use with the invention comprise the antibody sequences provided in FIGS. 61A-61P, also as provided in US 2020/040081 (incorporated herein by reference in its entirety)

In some embodiments, the anti-PVRIG antibodies for use with the invention can comprise CDRs identical to those shown in FIG. 24. In some embodiments, the anti-PVRIG antibody or binding portion thereof is as provided in WO 2017/041004 (incorporated herein by reference in its entirety). In some embodiments, the anti-PVRIG antibody or binding portion thereof is as provided in WO 2018/017864 (incorporated herein by reference in its entirety).

1. PVRIG Antibodies that Compete for Binding with Enumerated Antibodies

The present invention provides not only the enumerated antibodies but additional antibodies that compete with the enumerated antibodies (the CPA and CHA numbers enumerated herein that specifically bind to PVRIG) to specifically bind to the PVRIG molecule. The PVRIG antibodies for use with the invention “bin” into different epitope bins. There are four separate bins outlined herein; 1) the epitope bin into which CPA.7.002, CPA.7.003, CPA.7.005, CPA.7.007, CPA.7.010, CPA.7.012, CPA.7.015, CPA.7.016, CPA.7.017, CPA.7.019, CPA.7.020, CPA.7.021, CPA.7.024, CPA.7.028, CPA.7.032, CPA.7.033, CPA.7.036, CPA.7.037, CPA.7.038, CPA.7.043, CPA.7.046 and CPA.7.041 all fall into; 2) the epitope bin into which CPA.7.004, CPA.7.009, CPA.7.011, CPA.7.014, CPA.7.018, CPA.7.022, CPA.7.023, CPA.7.034, CPA.7.040, CPA.7.045 and CPA.7.047 all fall into; 3) CPA.7.039, which defines the distinction between bin 1 and bin 2, in that bin 1 blocks CPA.7.039 binding and bin 2 sandwiches the ligand with CPA.7.039, and bin 4) with CPA.7.050. The anti-PVRIG antibodies for use with the invention can comprise PVRIG antibodies and/or antigen binding domains sequences that are capable of competing with the enumerated antibodies (the CPA and CHA numbers enumerated herein that specifically bind to PVRIG) as part of the PVRIG binding portion.

Thus, the invention provides anti-PVRIG antibodies, where the PVRIG binding portion of the anti-PVRIG antibodies is capable of competing for binding with antibodies that are in bin 1, with antibodies that are in bin 2, with antibodies that are inbin 3 and/or with antibodies that are in bin 4.

Additional anti-PVRIG antibodies that compete with the enumerated antibodies are generated, as is known in the art and generally outlined below. Competitive binding studies can be done as is known in the art, generally using SPR/Biacore® binding assays, as well as ELISA and cell-based assays.

### B. Anti-TIGIT Antibodies

The anti-TIGIT antibodies described herein can comprise a TIGIT antibody and/or antigen binding domain sequence as part of the TIGIT binding portion, where the TIGIT antibodies are labeled as follows. Such TIGIT antibodies have reference numbers, for example “CPA.9.086”. This represents the combination of the variable heavy and variable light chains, as depicted in FIG. 22A-22D, for example, with the understanding that these antibodies include two heavy chains and two light chains. “CPA.9.086.VH” refers to the variable heavy portion of CPA.9.086, while “CPA.9.086.VL” is the variable light chain. “CPA.9.086.vhCDR1”, “CPA.9.086.vhCDR2”, “CPA.9.086.vhCDR3”, “CPA.9.086.vlCDR1”, “CPA.9.086.vlCDR2”, and “CPA.9.086.vlCDR3”, refers to the CDRs are indicated. “CPA.9.086.HC” refers to the entire heavy chain (e.g. variable and constant domain) of this molecule, and “CPA.9.086.LC” refers to the entire light chain (e.g. variable and constant domain) of the same molecule. In general, the human kappa light chain is used for the constant domain of each phage (or humanized hybridoma) antibody herein, although in some embodiments the lambda light constant domain is used. “CPA.9.086.H1” refers to a full length antibody comprising the variable heavy and light domains, including the constant domain of Human IgG1 (hence, the H1; IgG1, IgG2, IgG3 and IgG4 sequences are shown in FIGS. 9A-9C and 21A-21B). Accordingly, “CPA.9.086.H2” would be the CPA.9.086 variable domains linked to a Human IgG2. “CPA.9.086.H3” would be the CPA.9.086 variable domains linked to a Human IgG3, and “CPA.9.086.H4” would be the CPA.9.086 variable domains linked to a Human IgG4. Note that in some cases, the human IgGs may have additional mutations, such are described below, and this can be annotated. For example, in many embodiments, there may be a S241P mutation in the human IgG4, and this can be annotated as “CPA. 9.086.H4(S241P)” for example. The human IgG4 sequence with this S241P hinge variant is shown in FIG. 20A-20B. Other potential variants are IgG1(N297A), (or other variants that ablate glycosylation at this site and thus many of the effector functions associated with FcγRIIIa binding), and IgG1(D265A), which reduces binding to FcγR receptors. The anti-TIGIT antibodies for use with the invention can comprise any of the TIGIT antibody domain sequences as the TIGIT binding portion of the anti-TIGIT antibodies. The anti-TIGIT antibodies for use with the invention can comprise any of the TIGIT antigen binding domains as the TIGIT binding portion of the anti-TIGIT antibodies.

The invention further provides variable heavy and light domains as well as full length heavy and light chains.

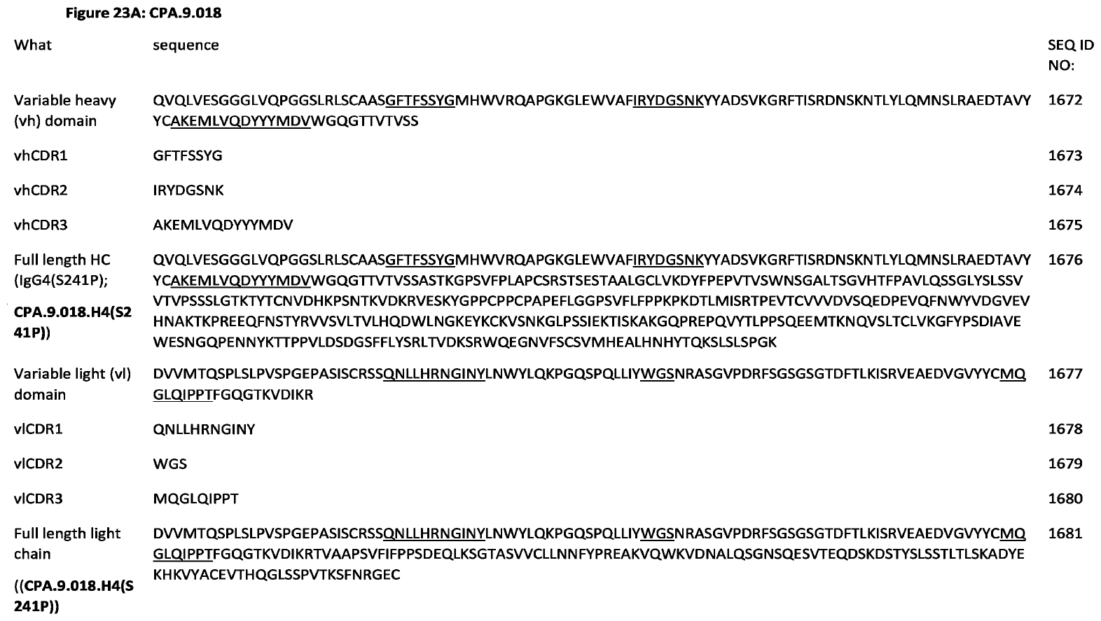

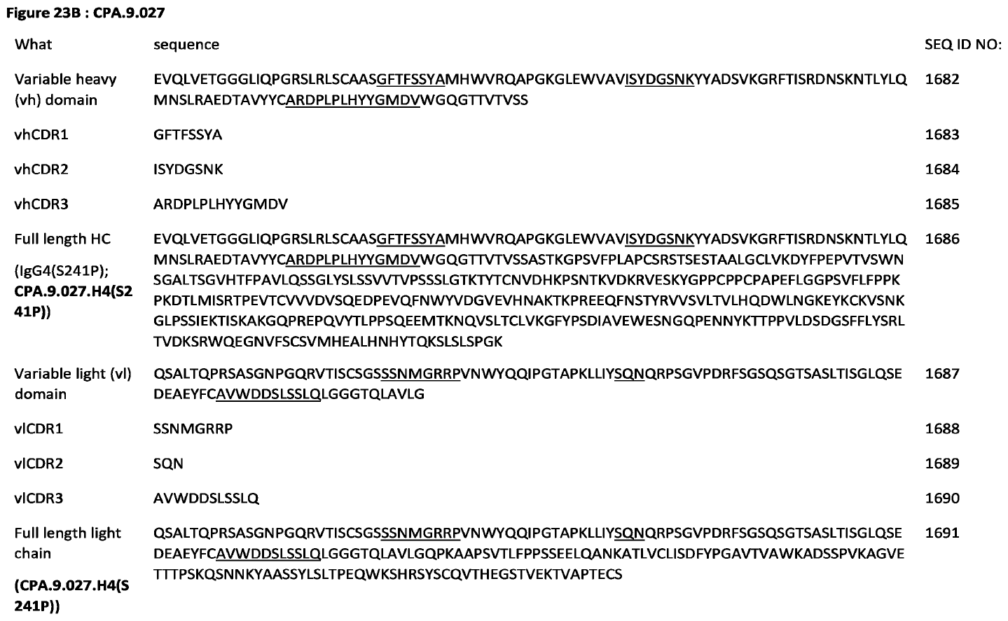

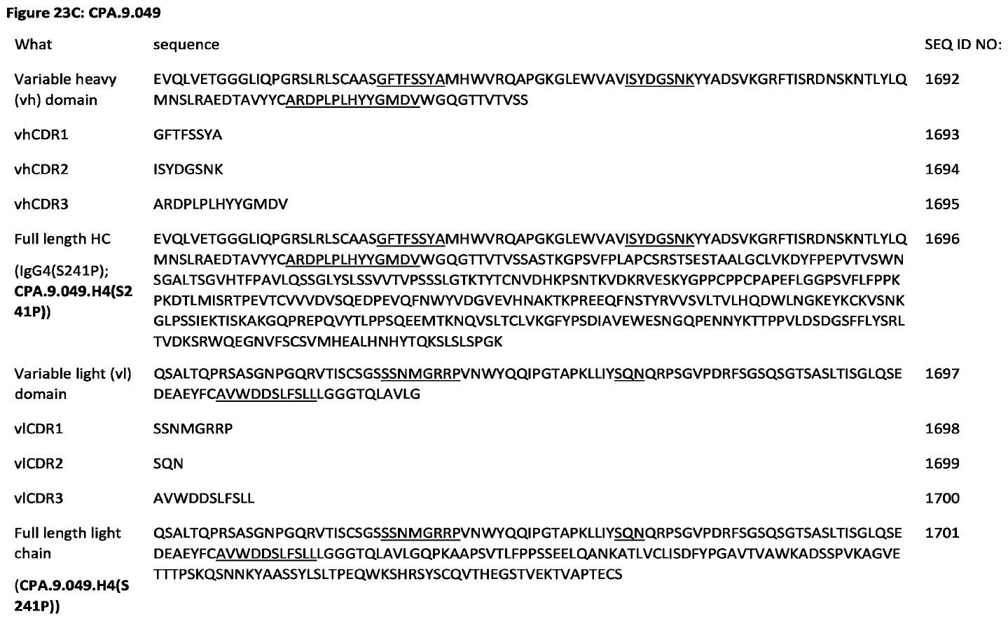

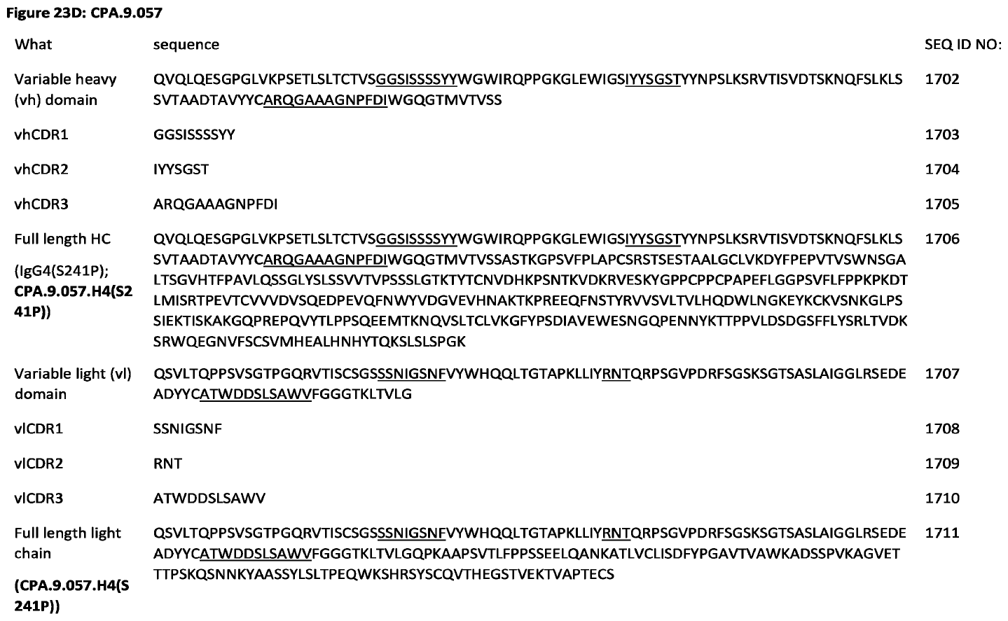

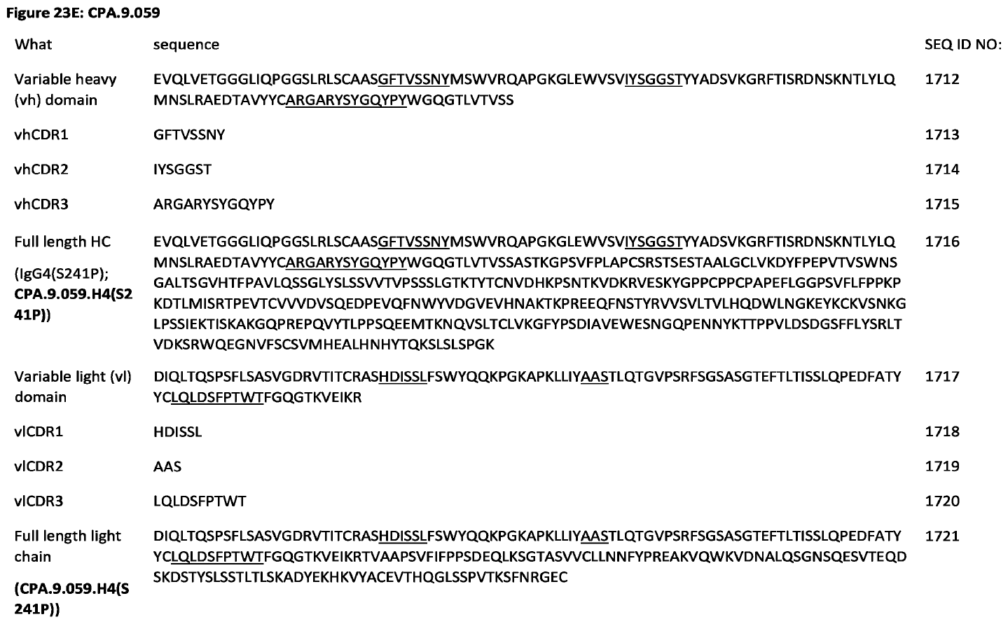

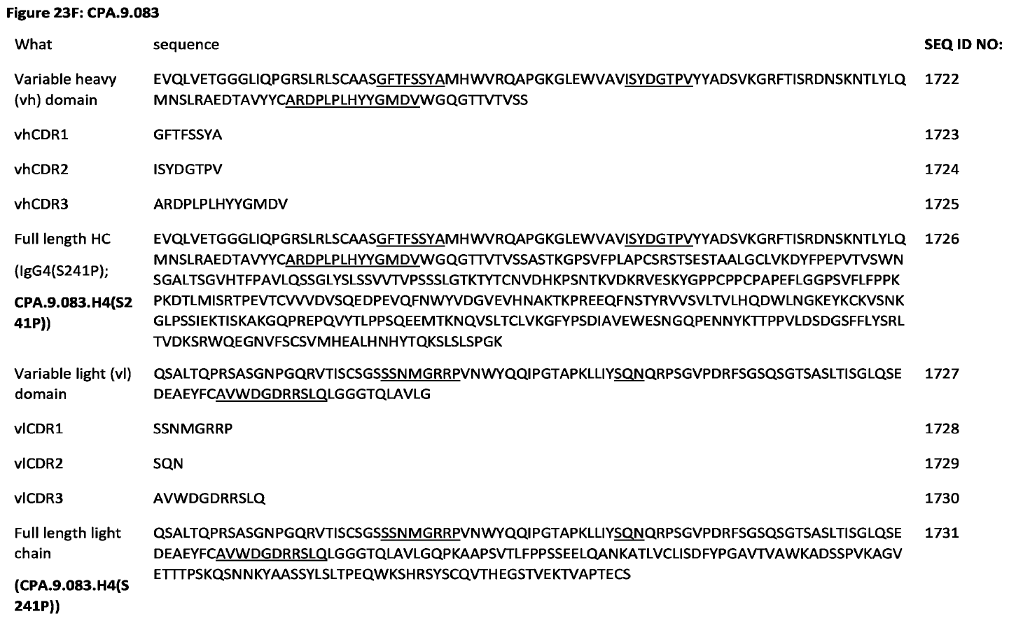

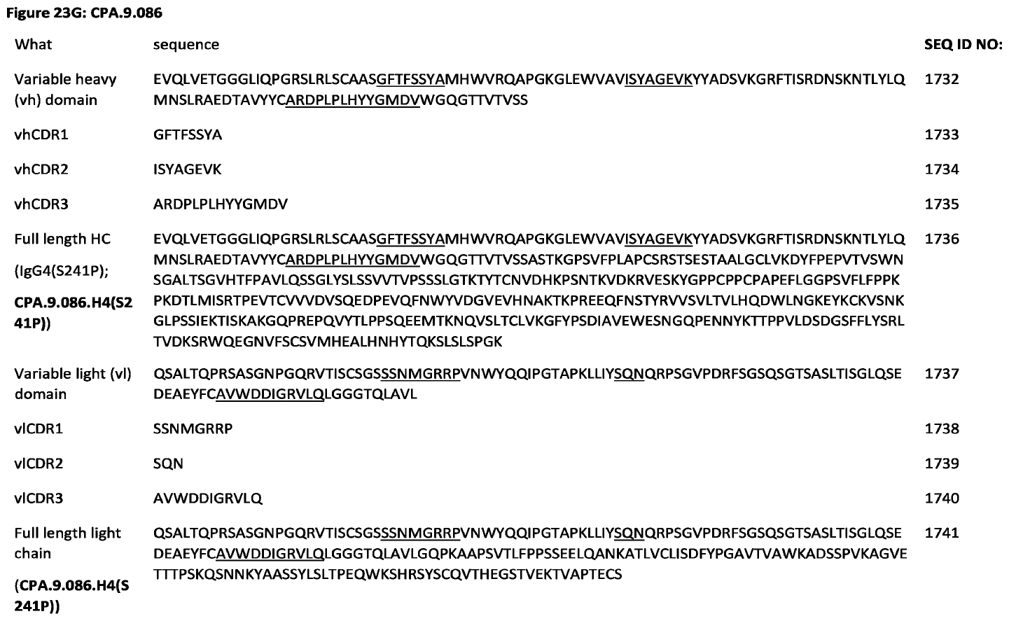

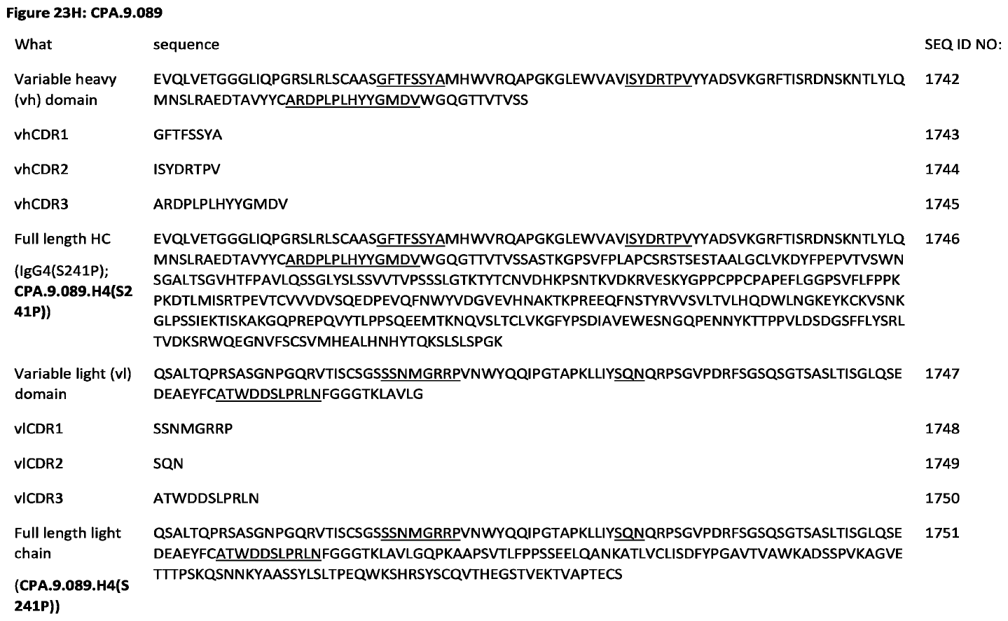

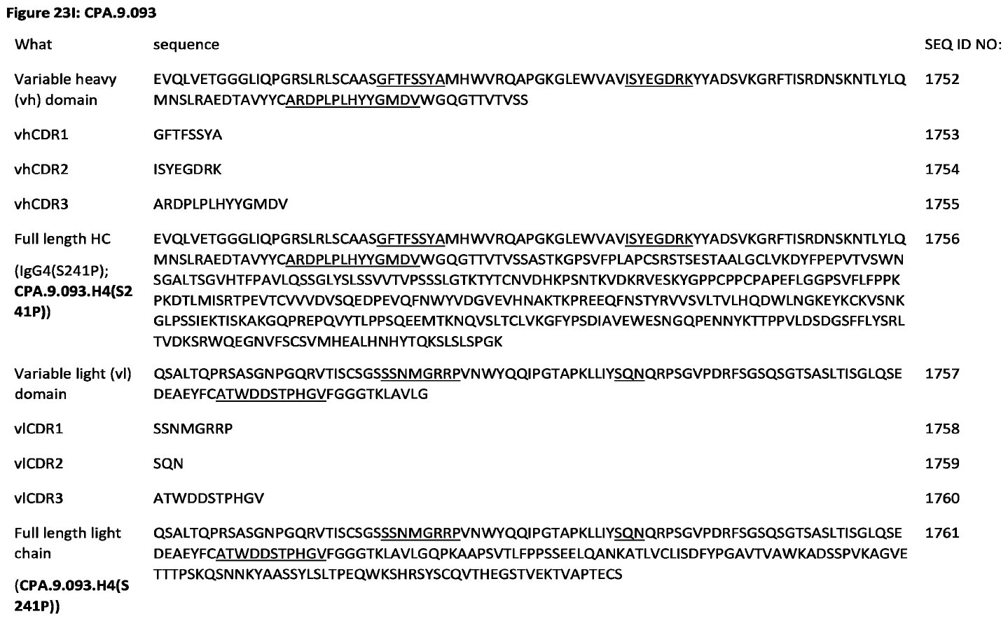

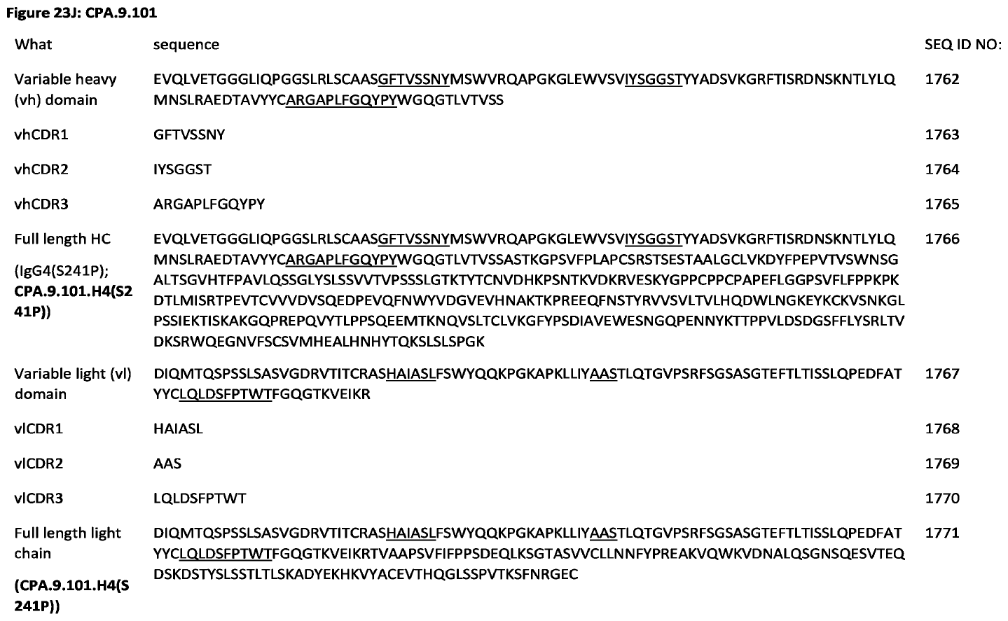

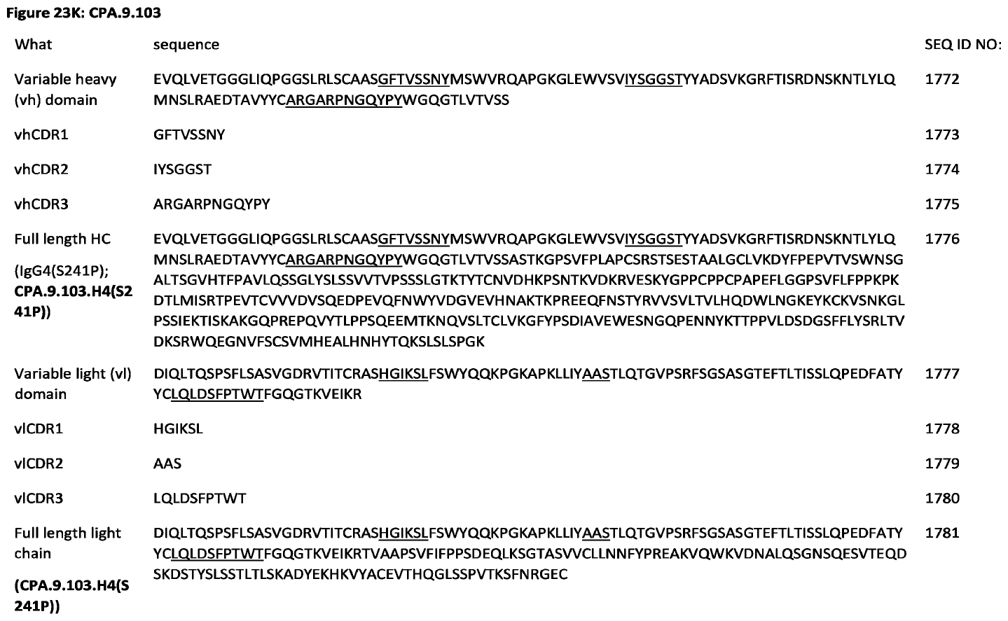

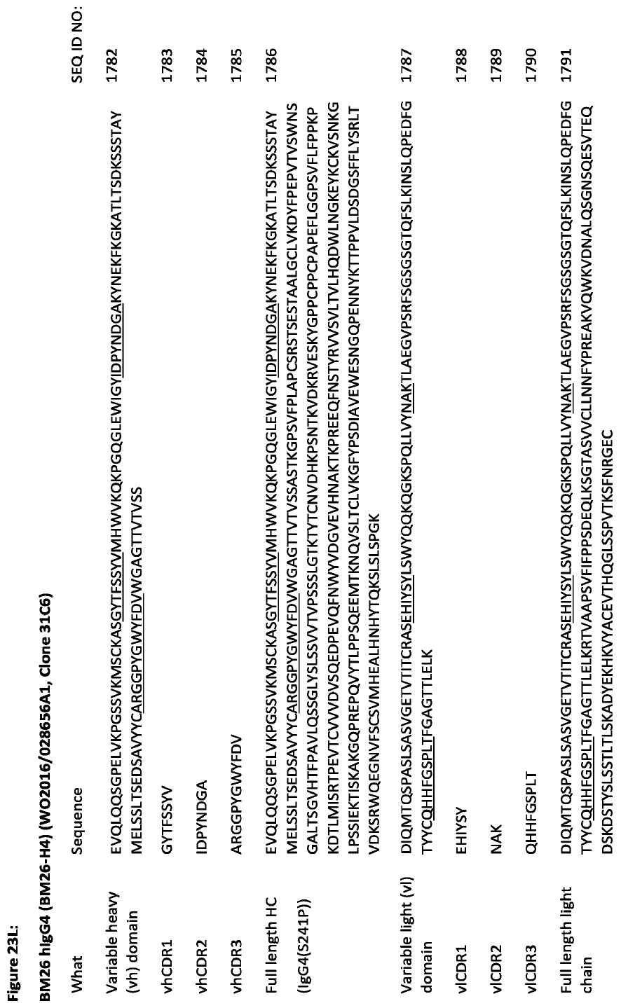

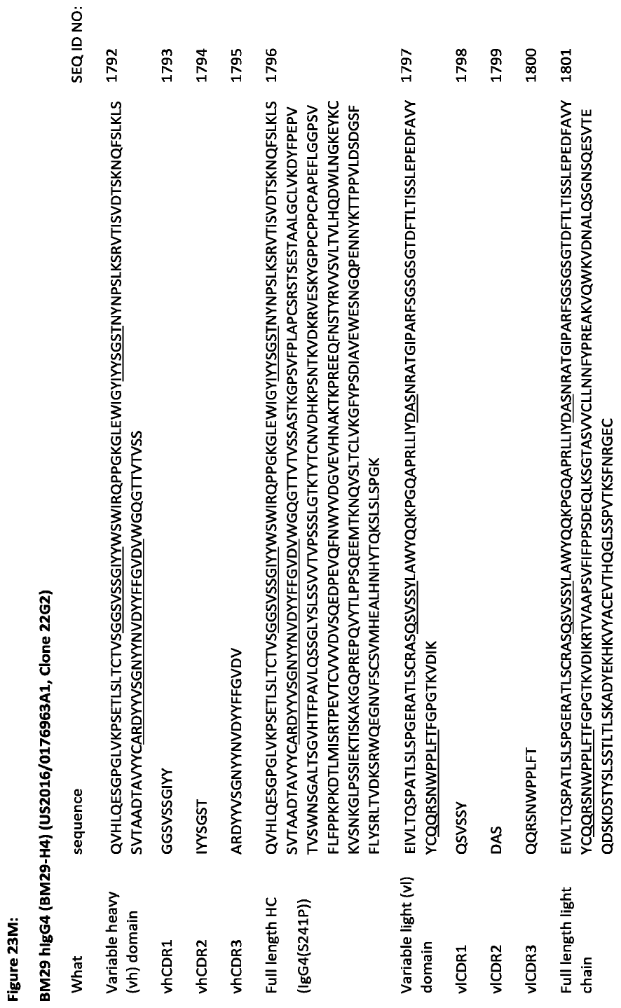

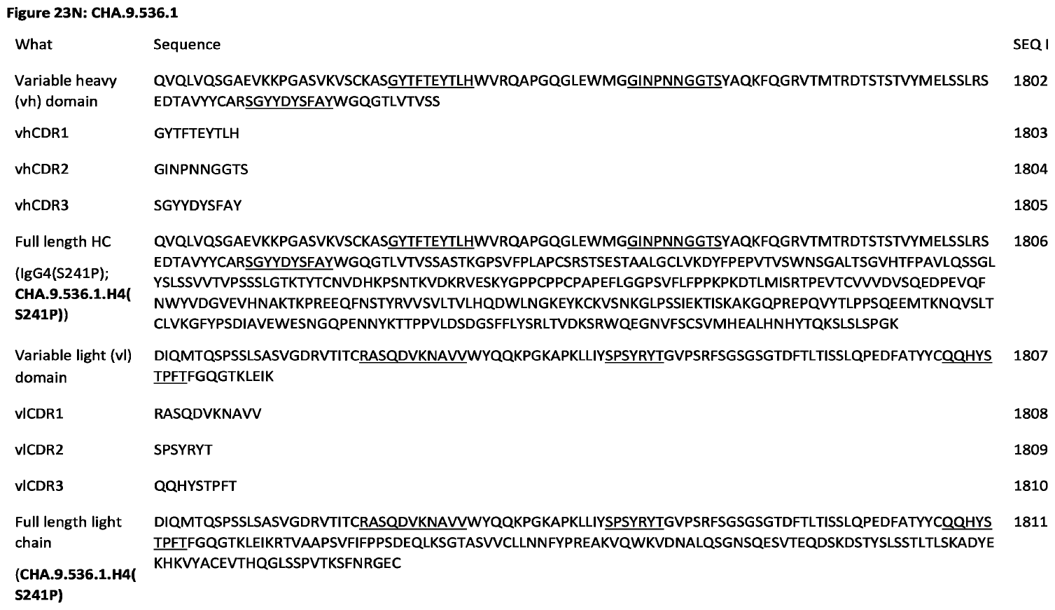

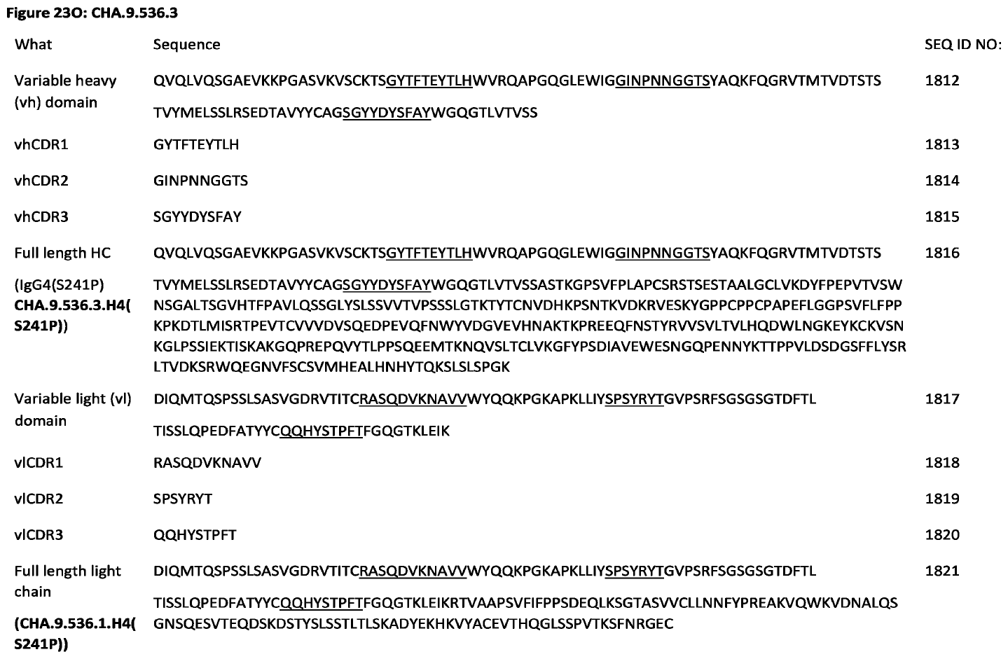

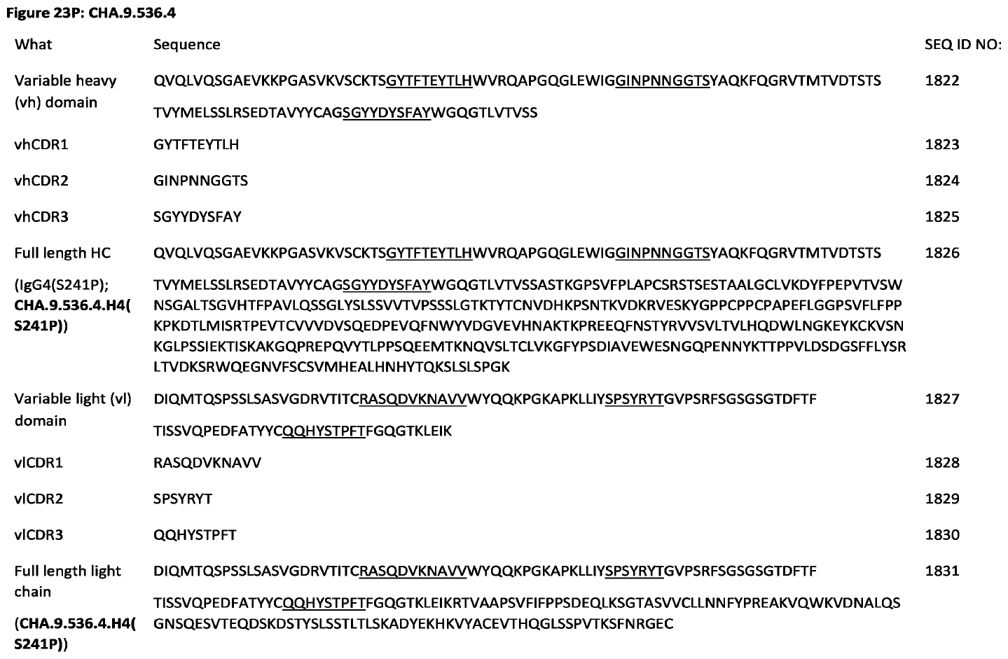

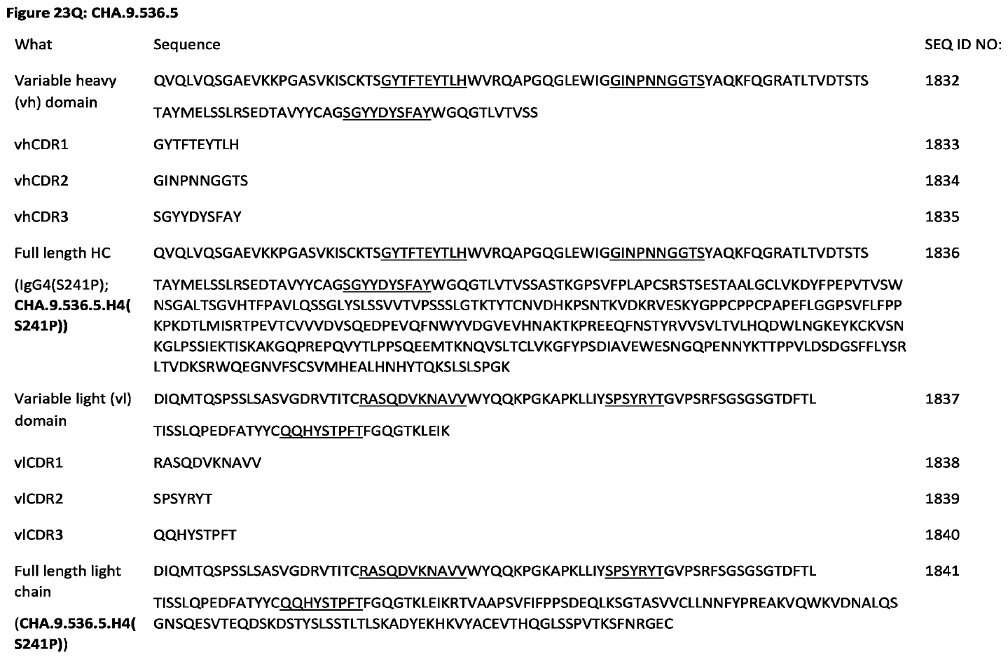

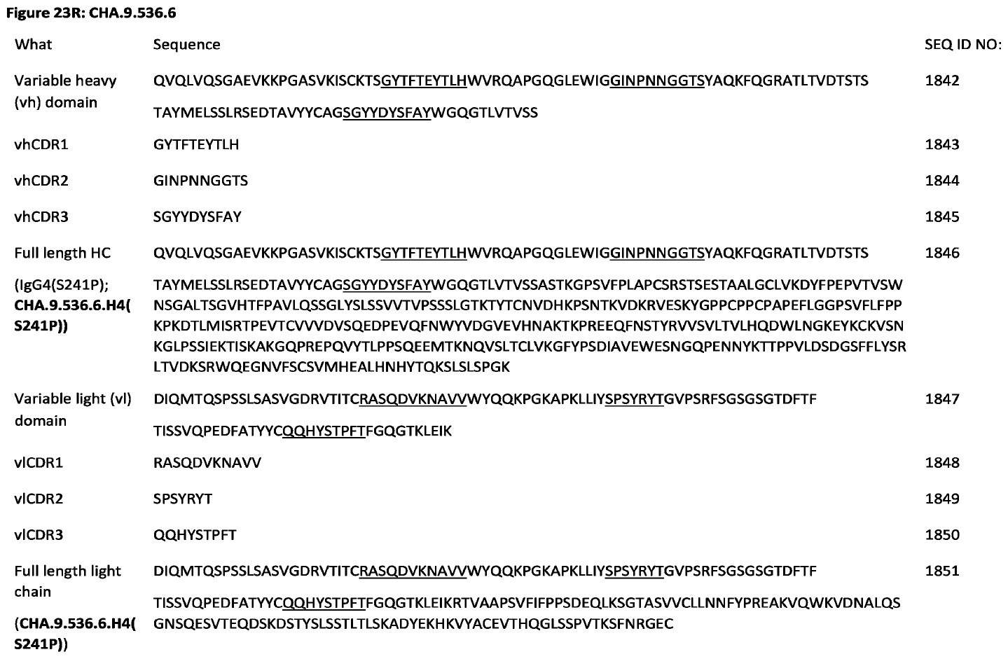

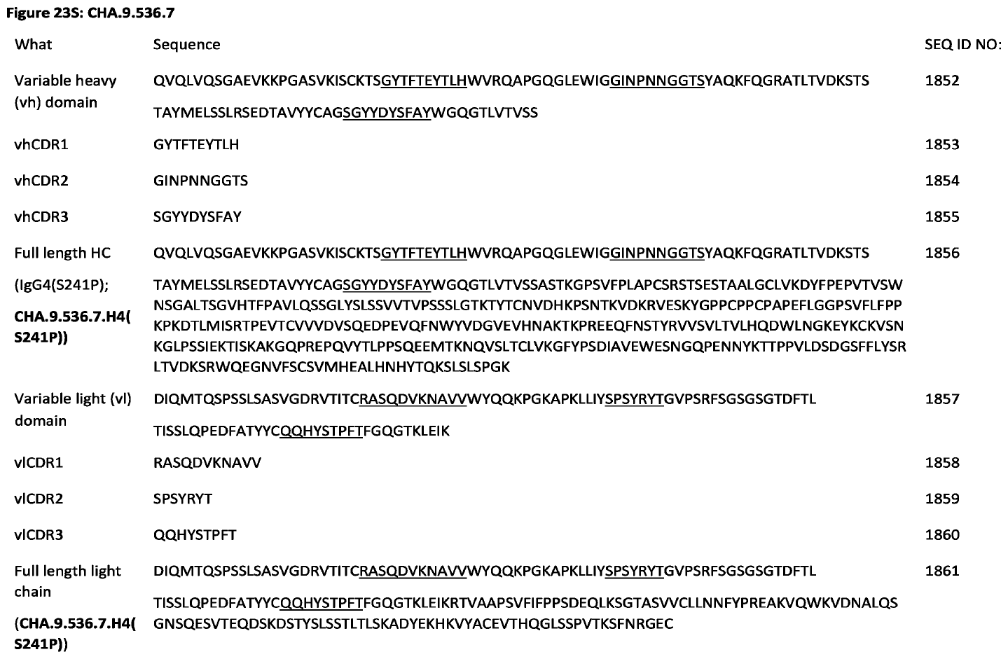

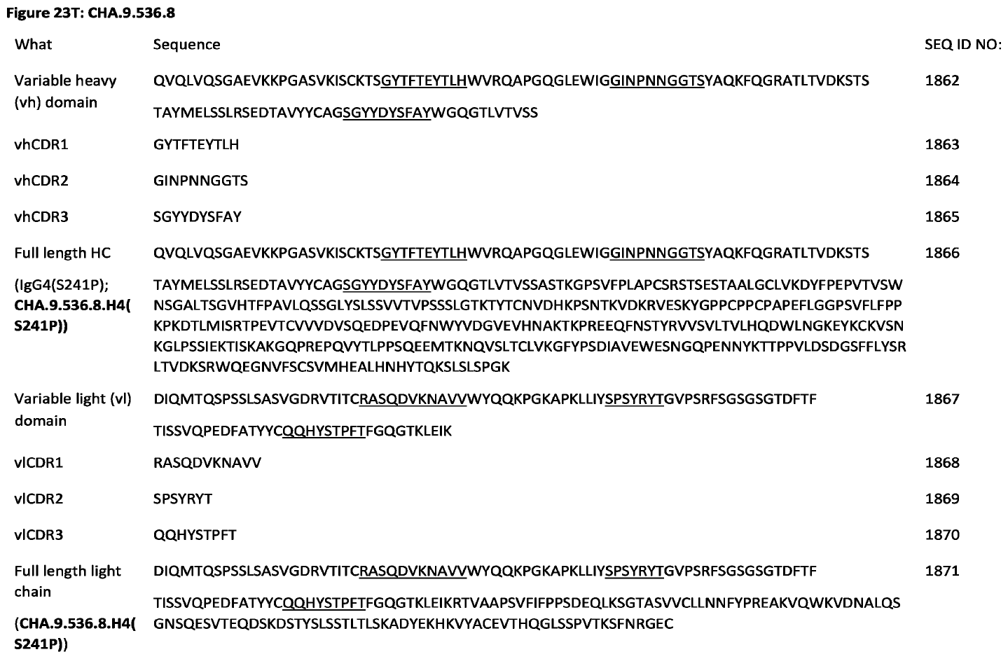

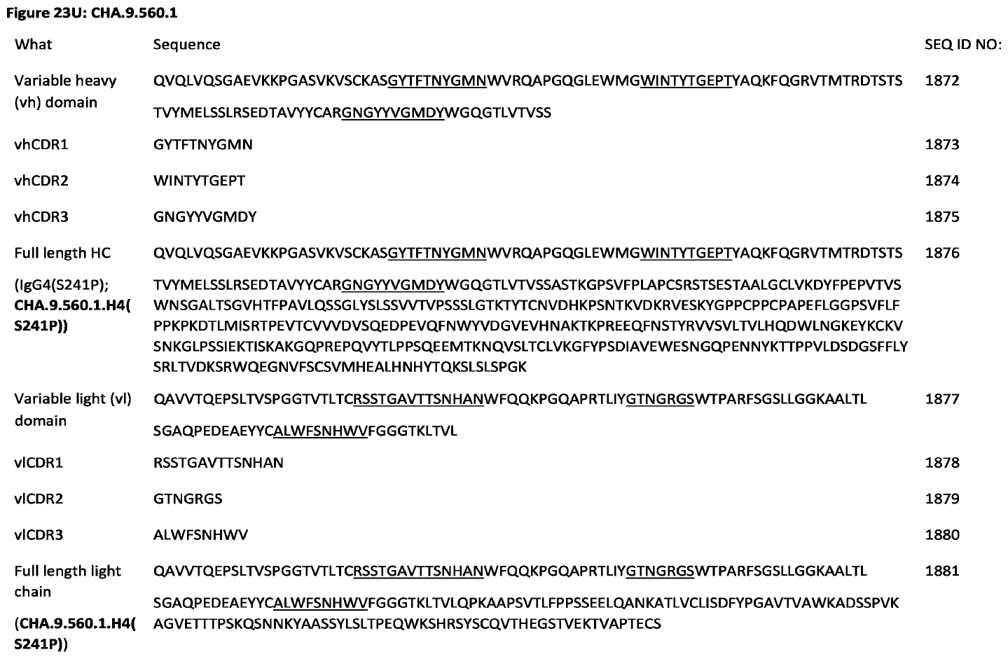

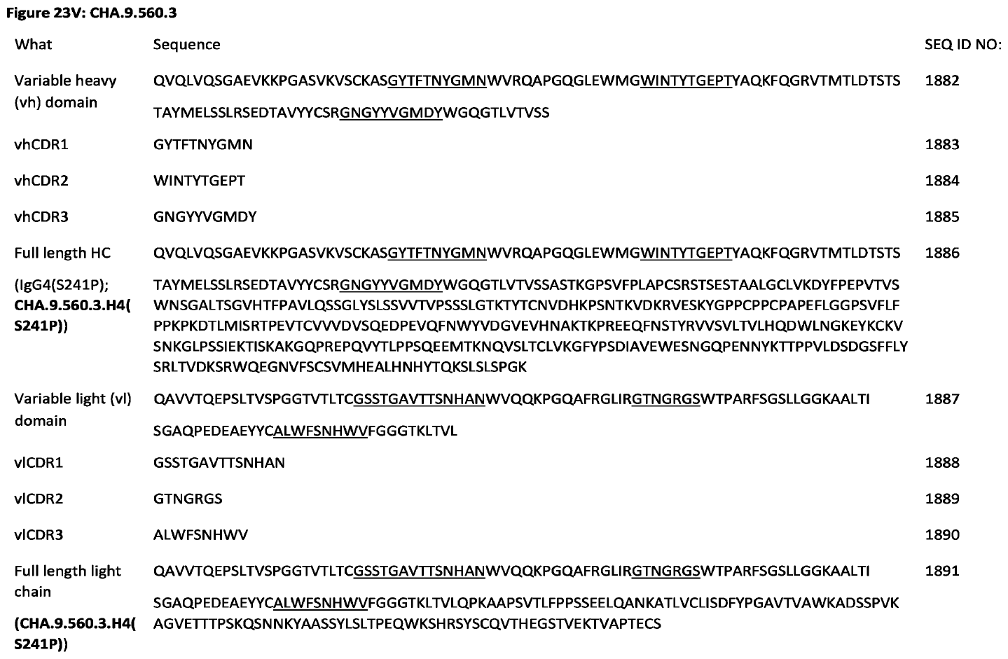

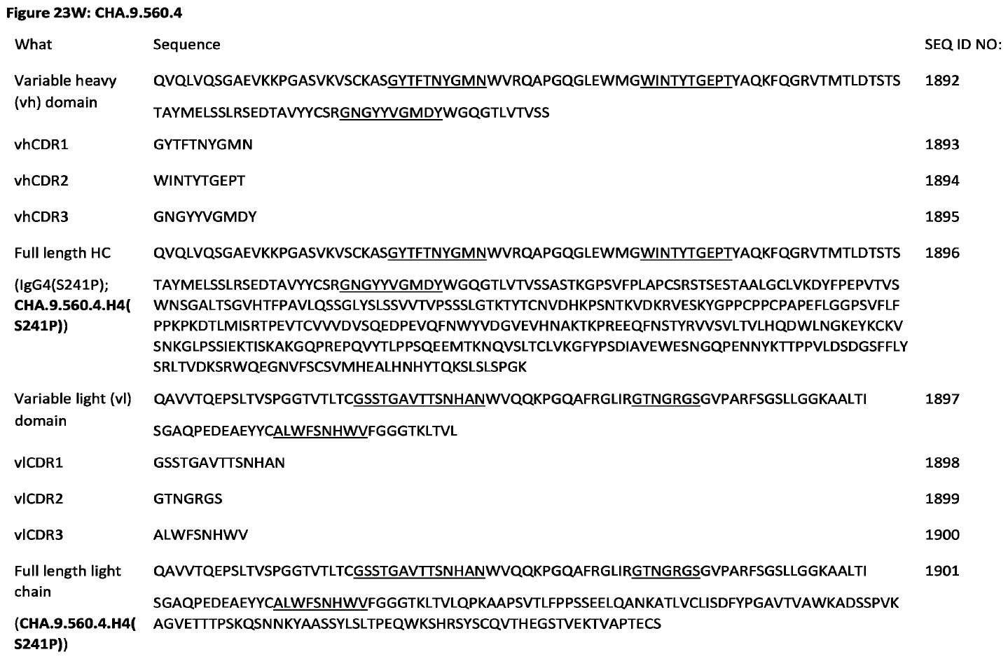

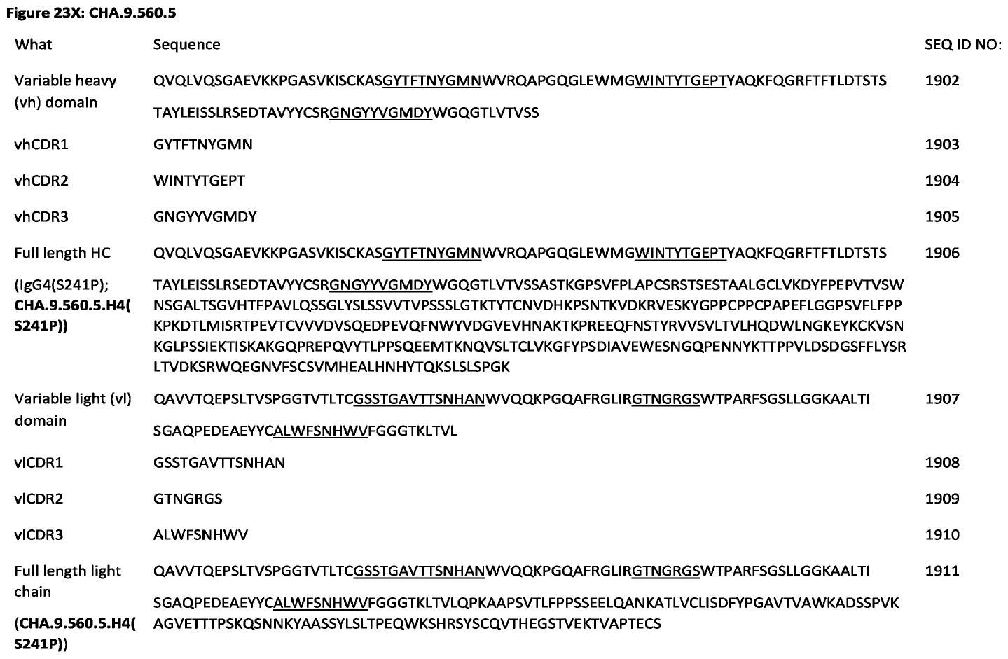

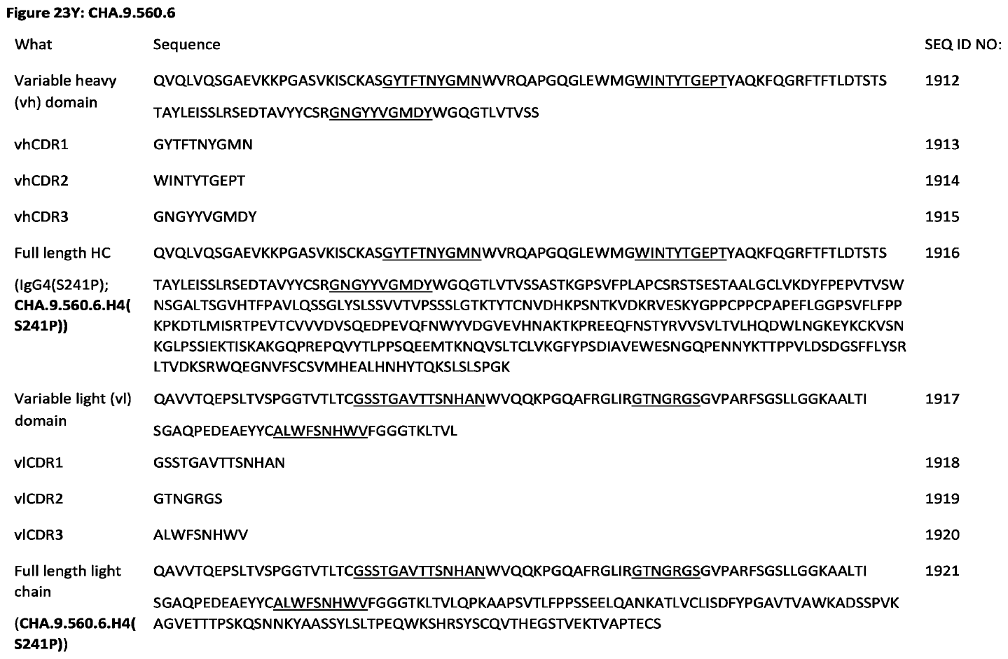

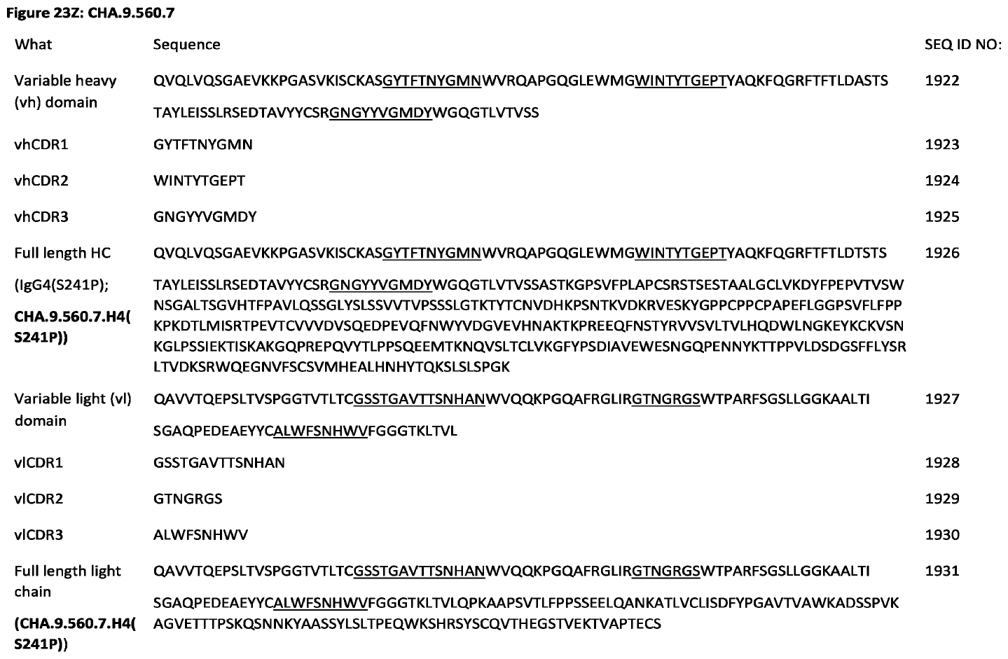

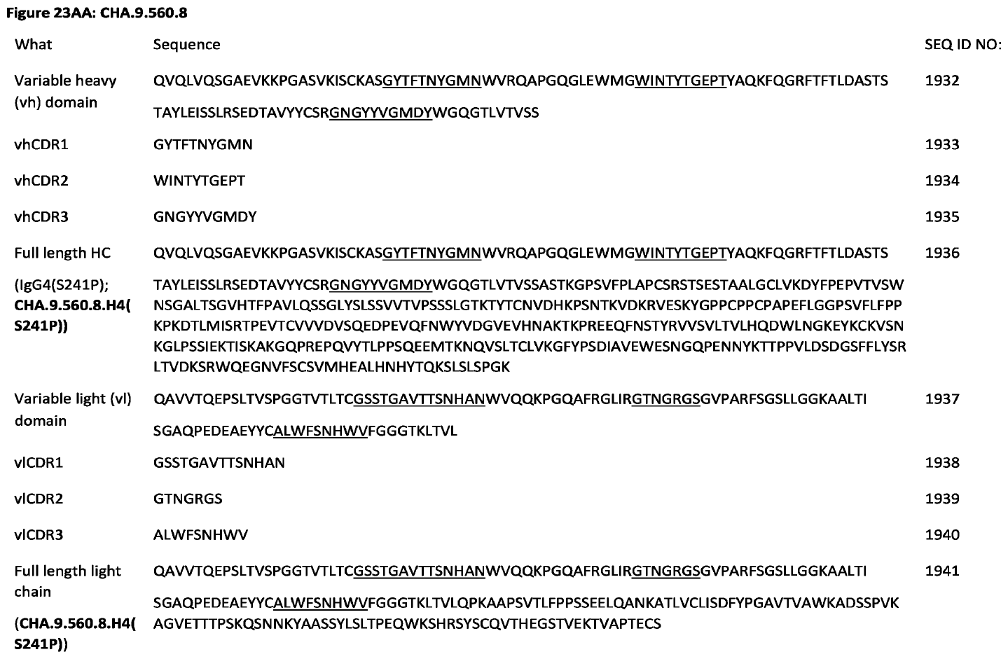

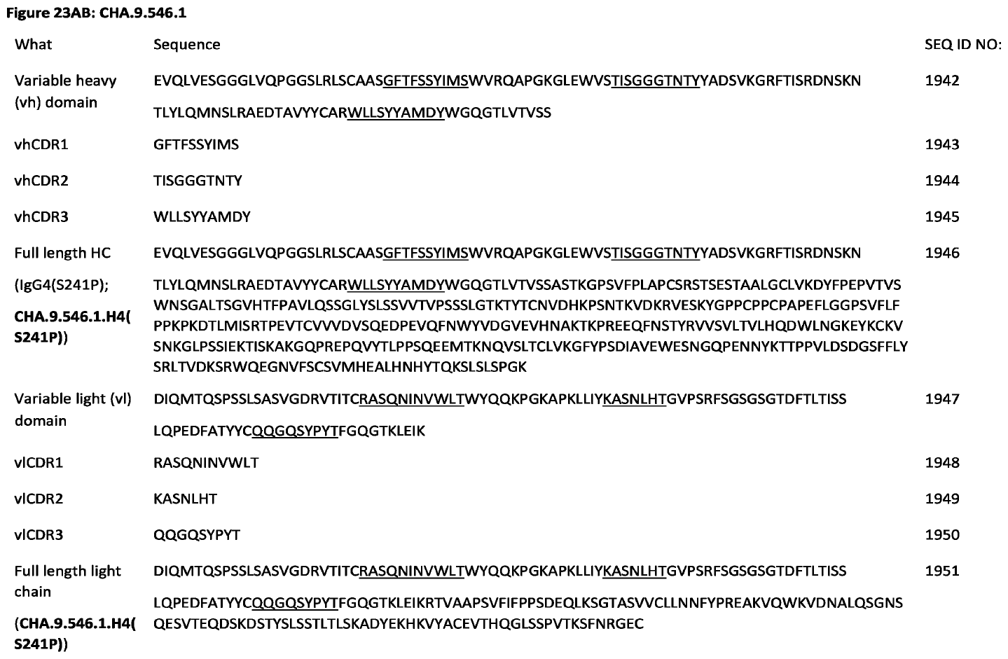

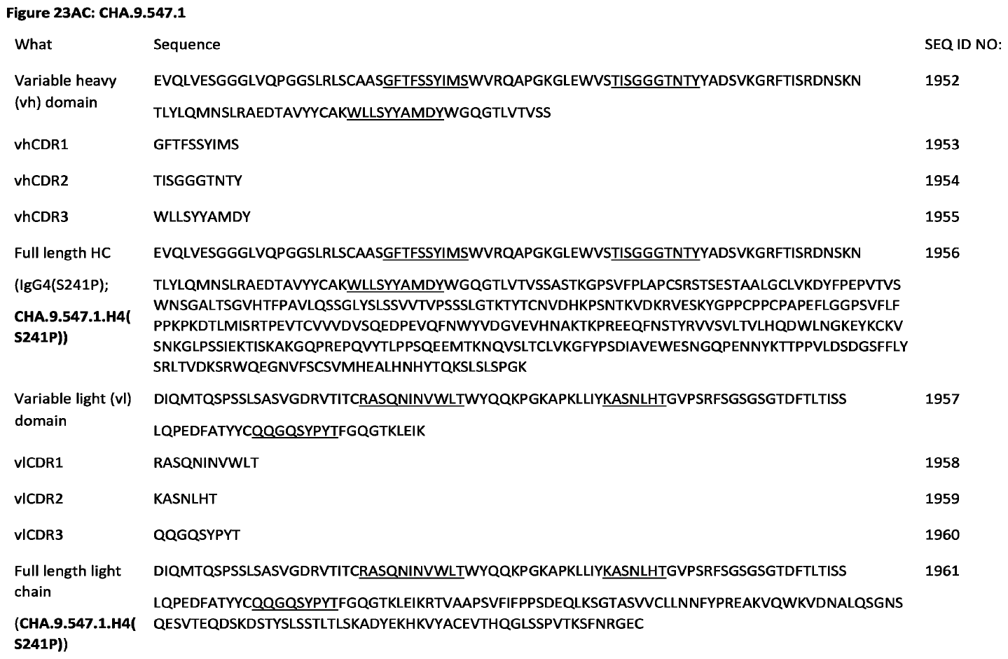

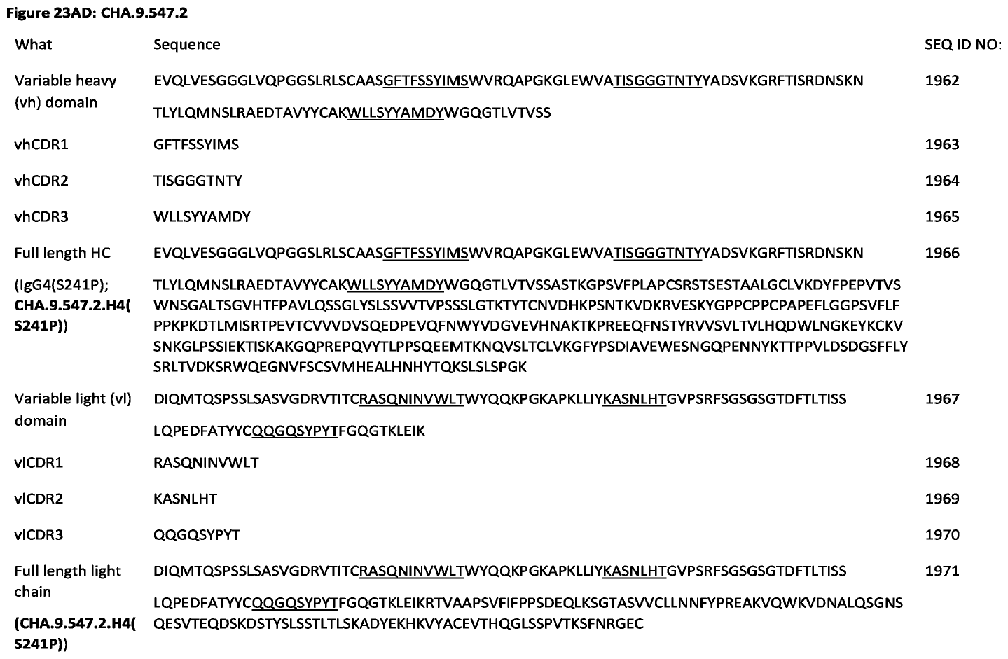

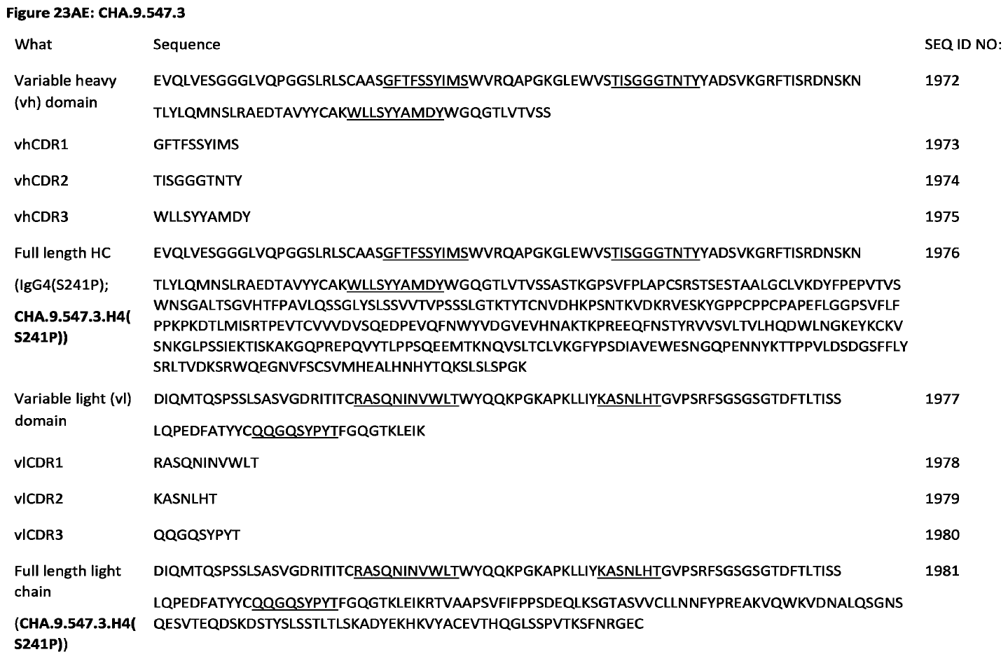

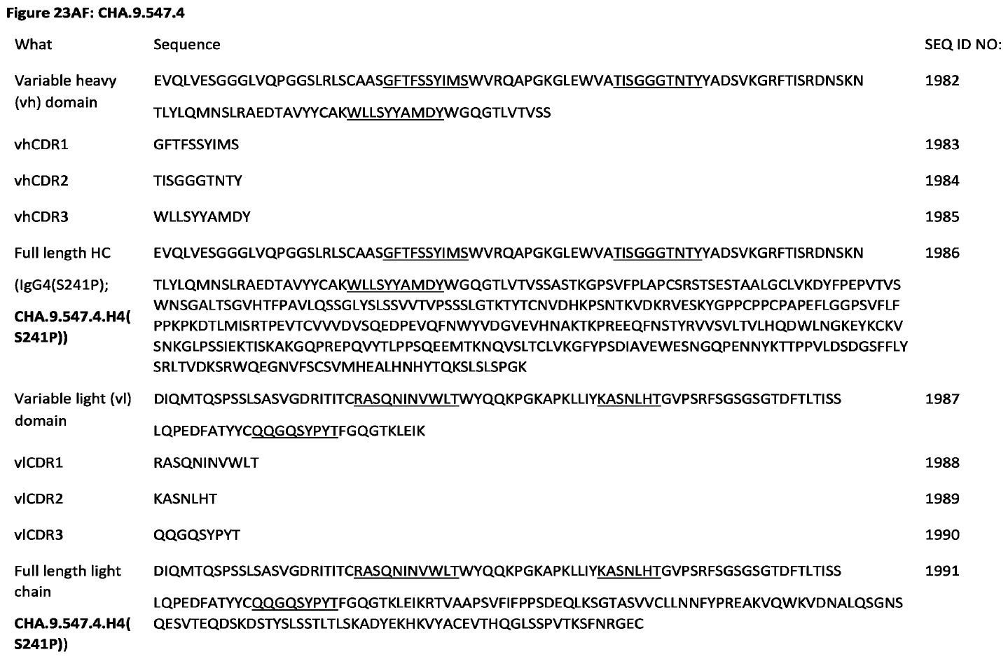

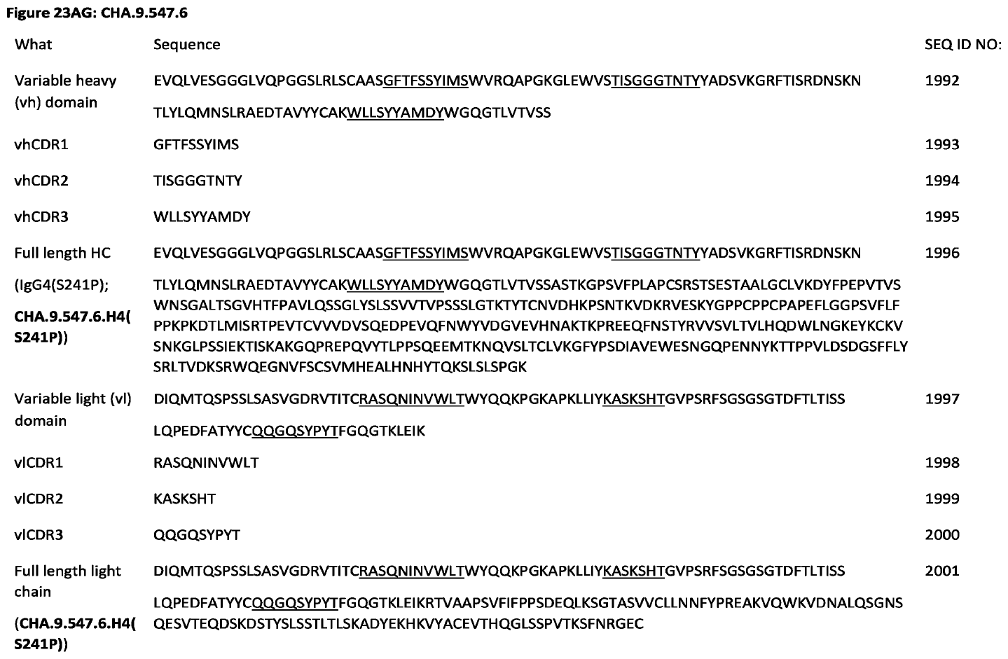

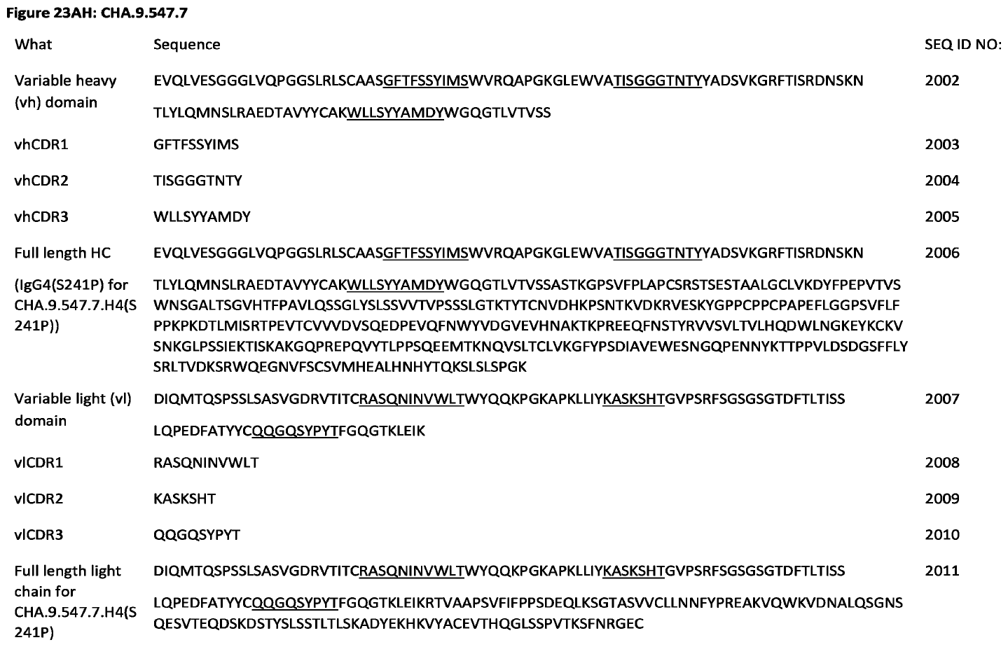

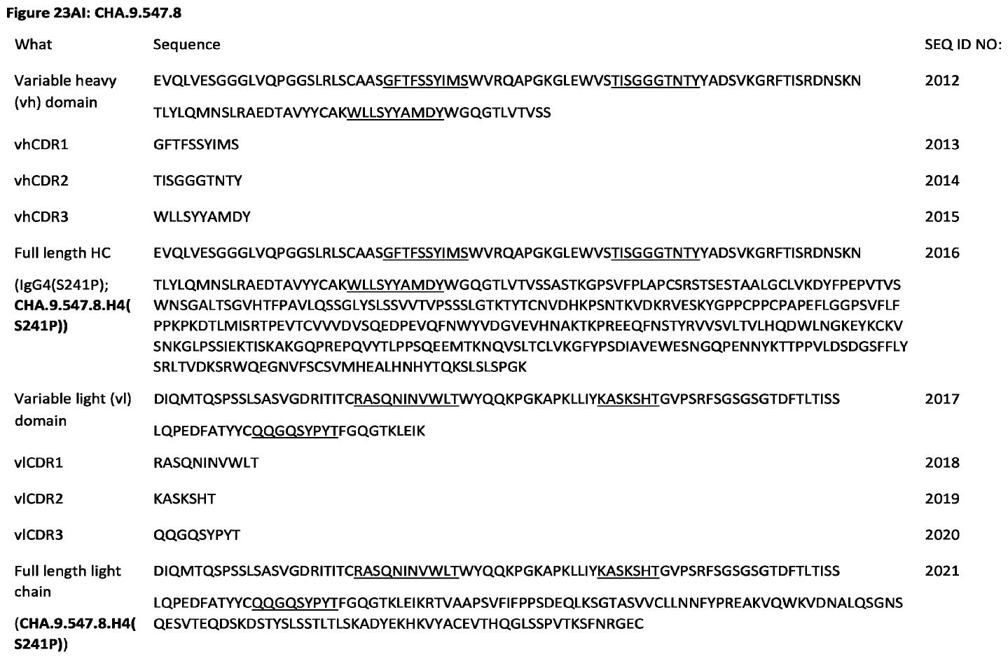

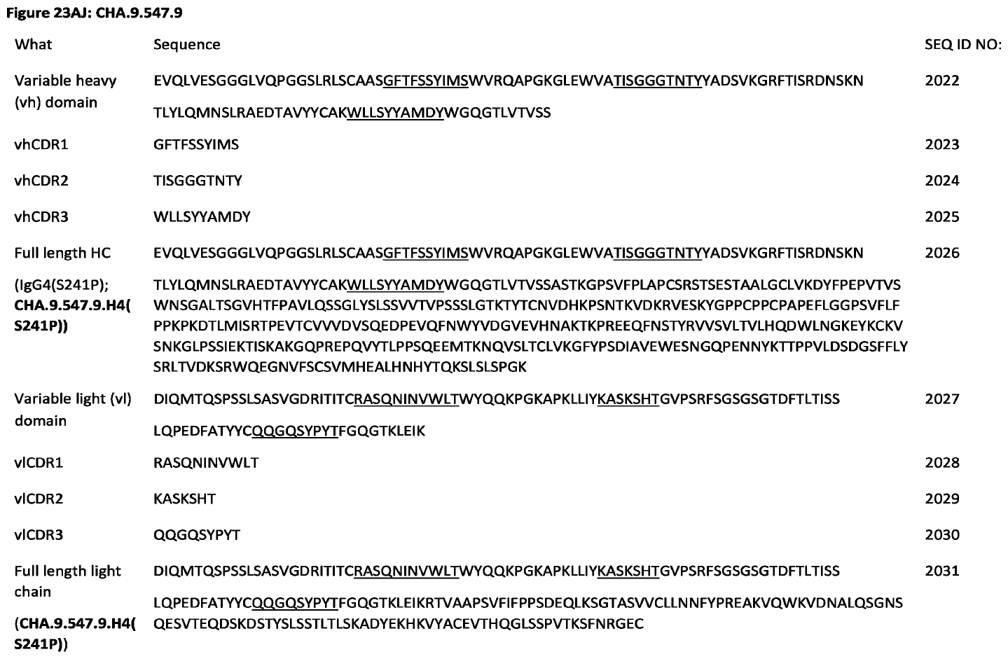

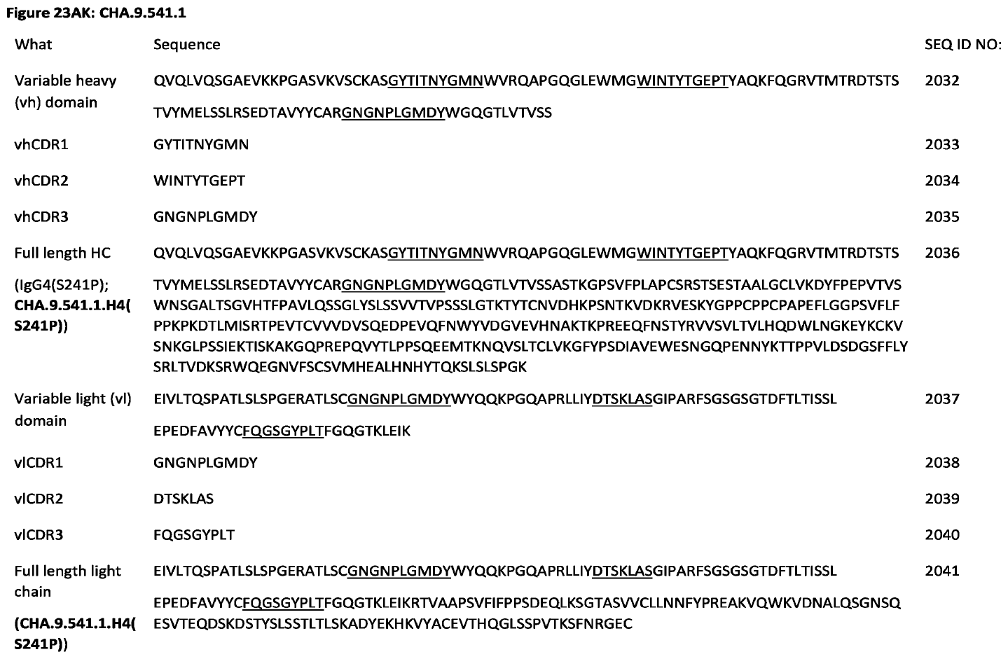

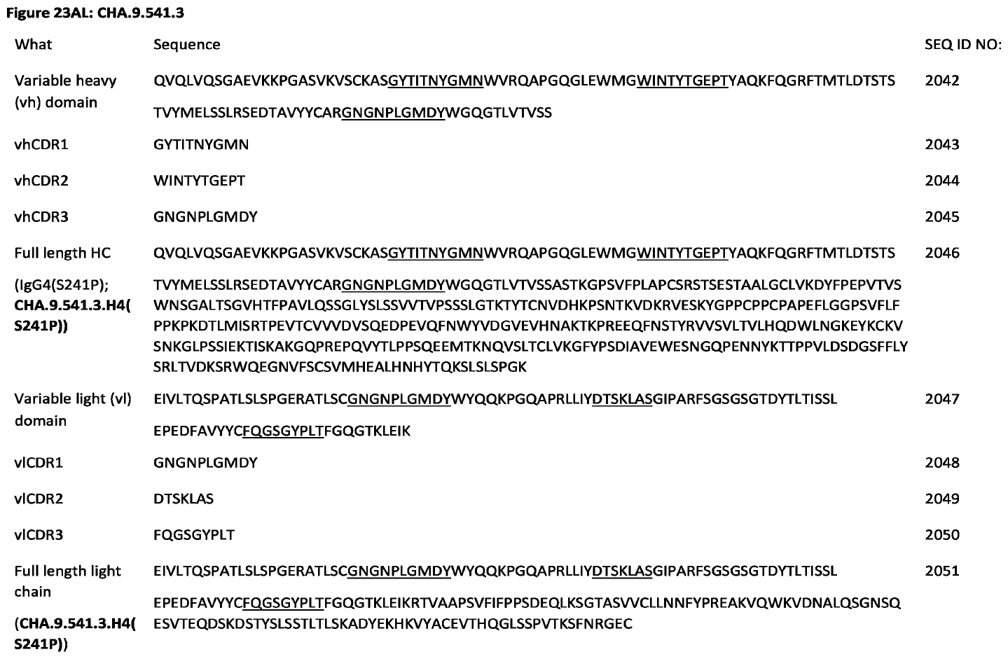

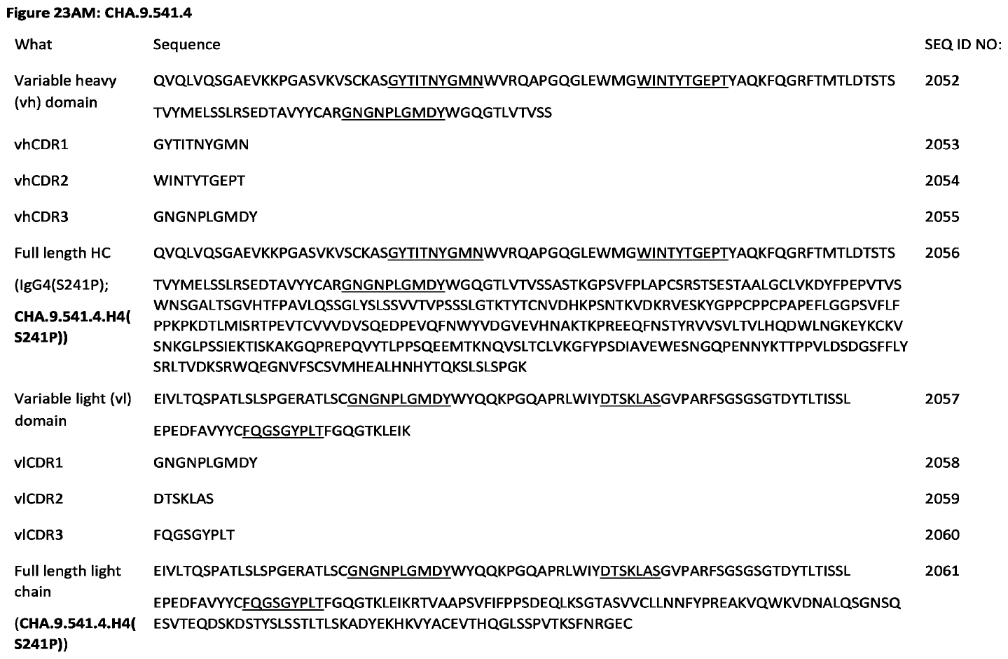

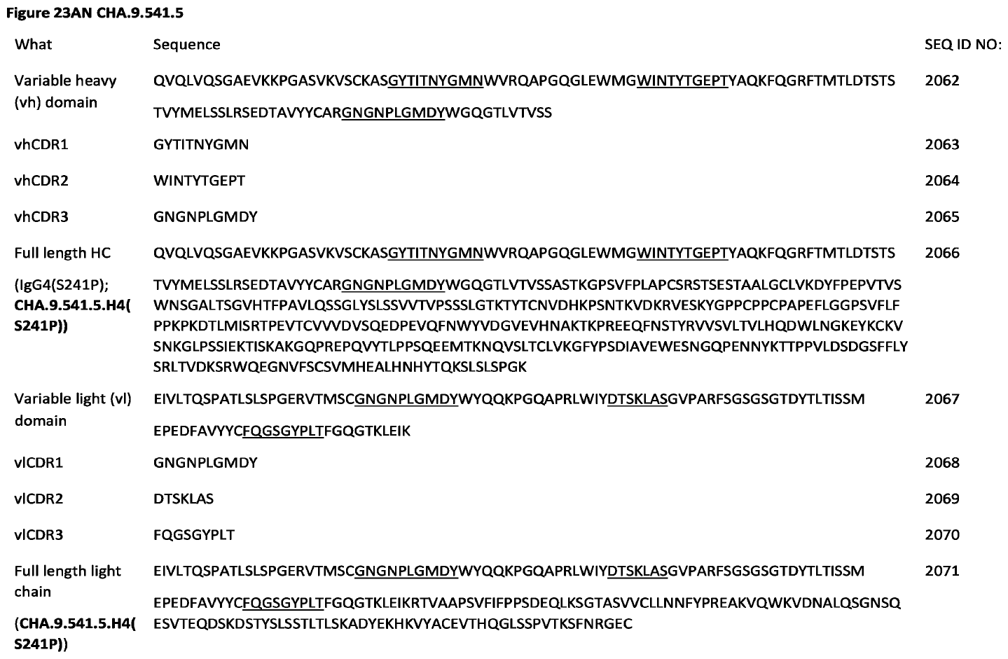

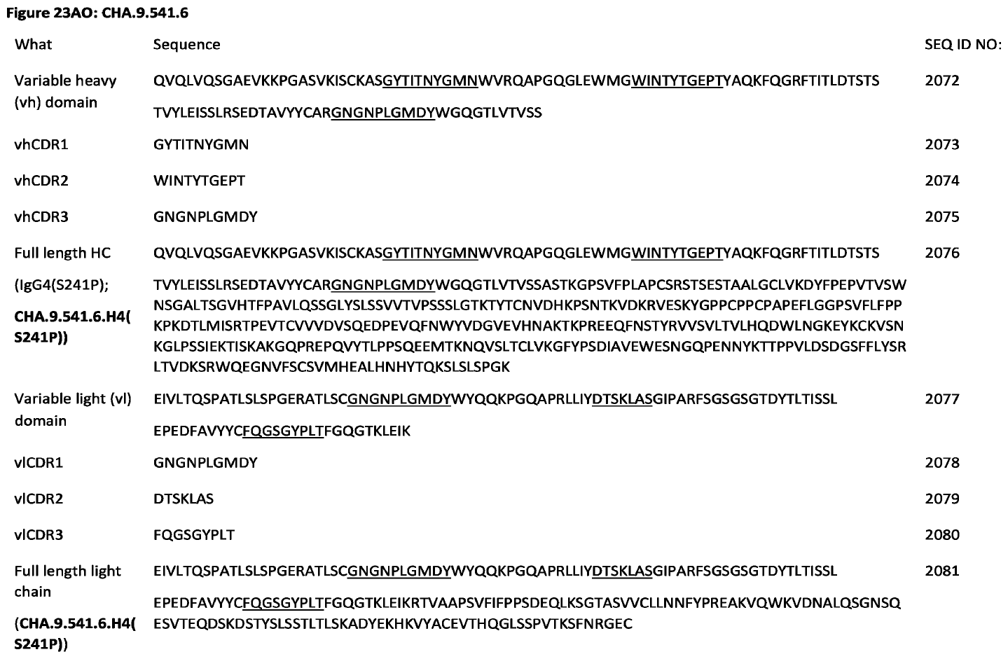

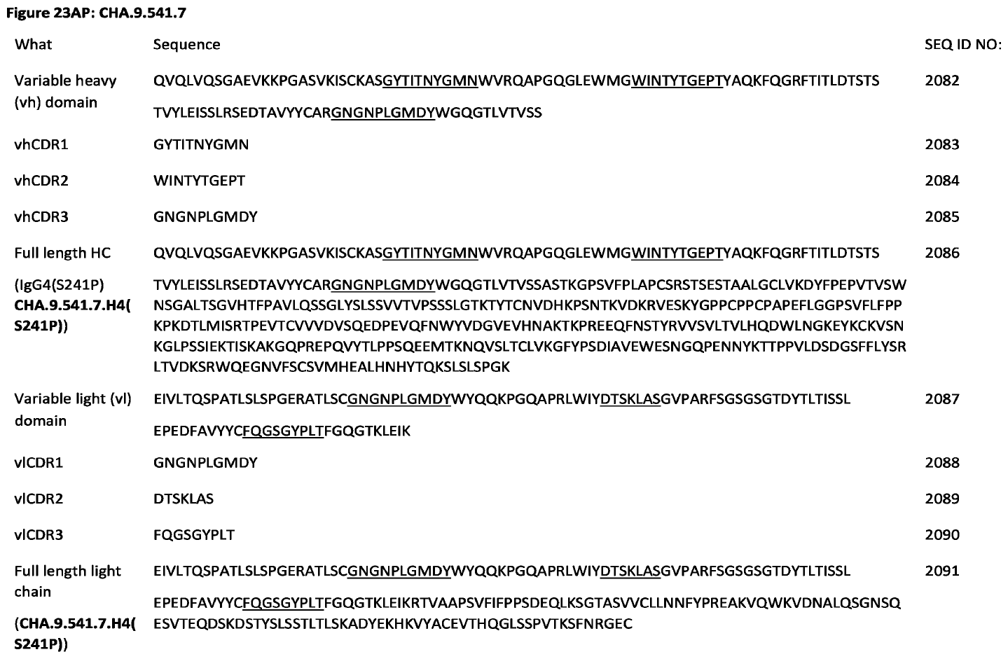

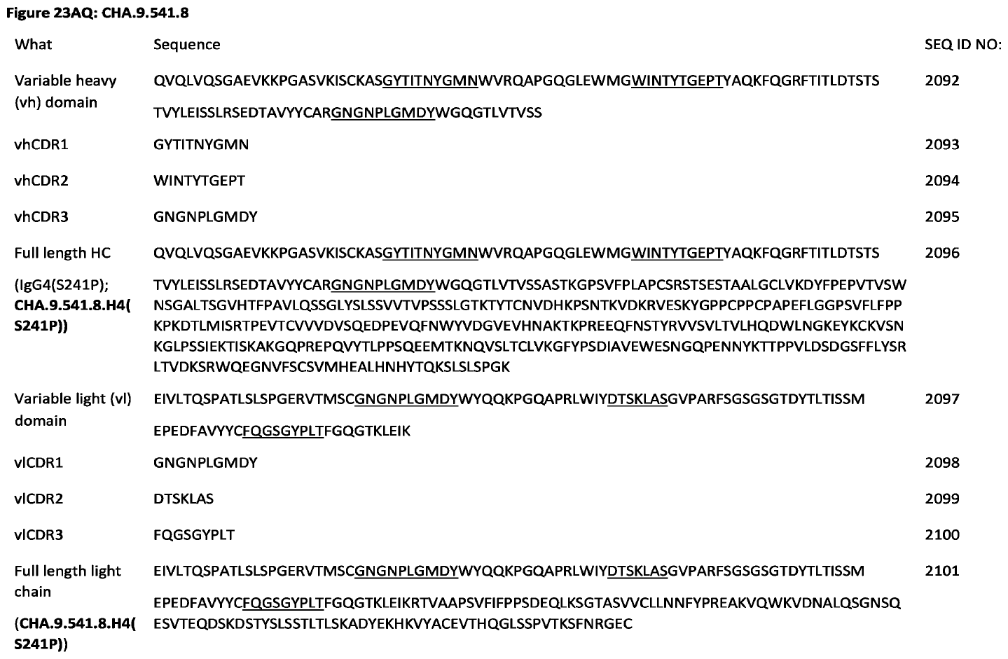

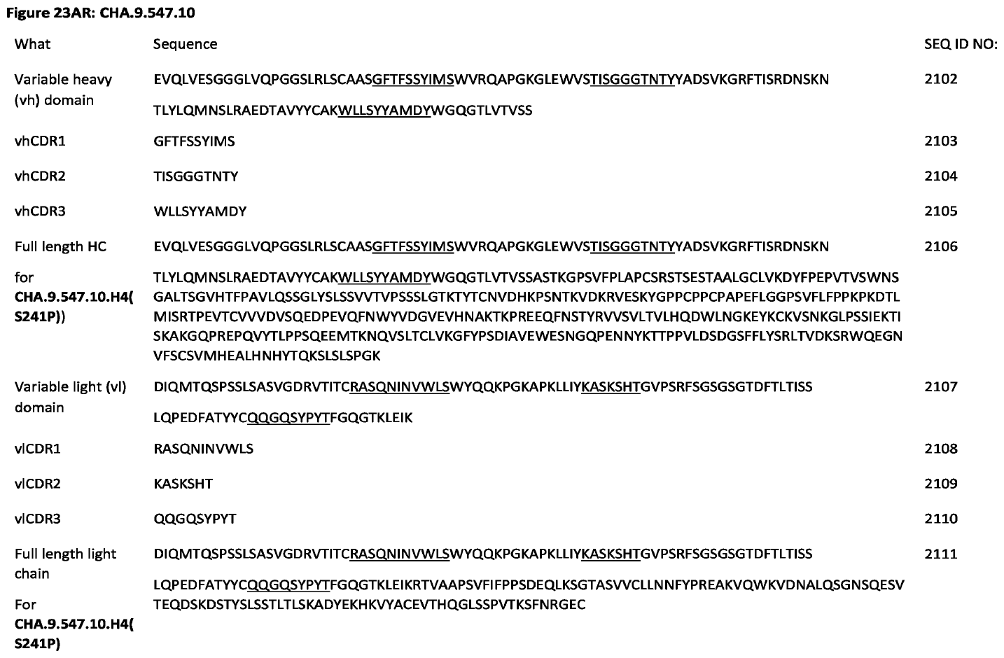

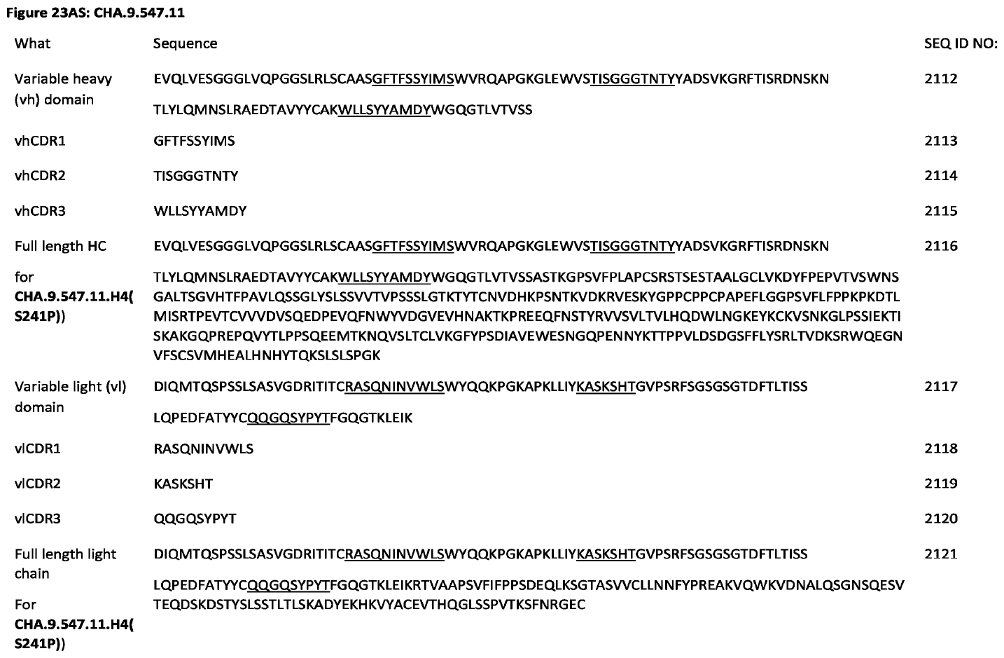

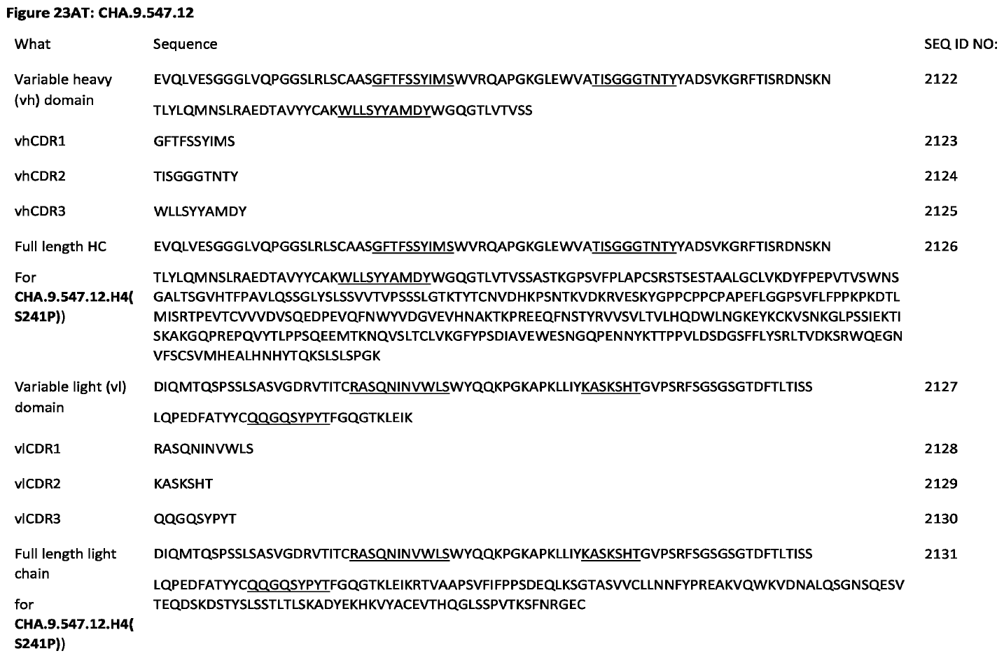

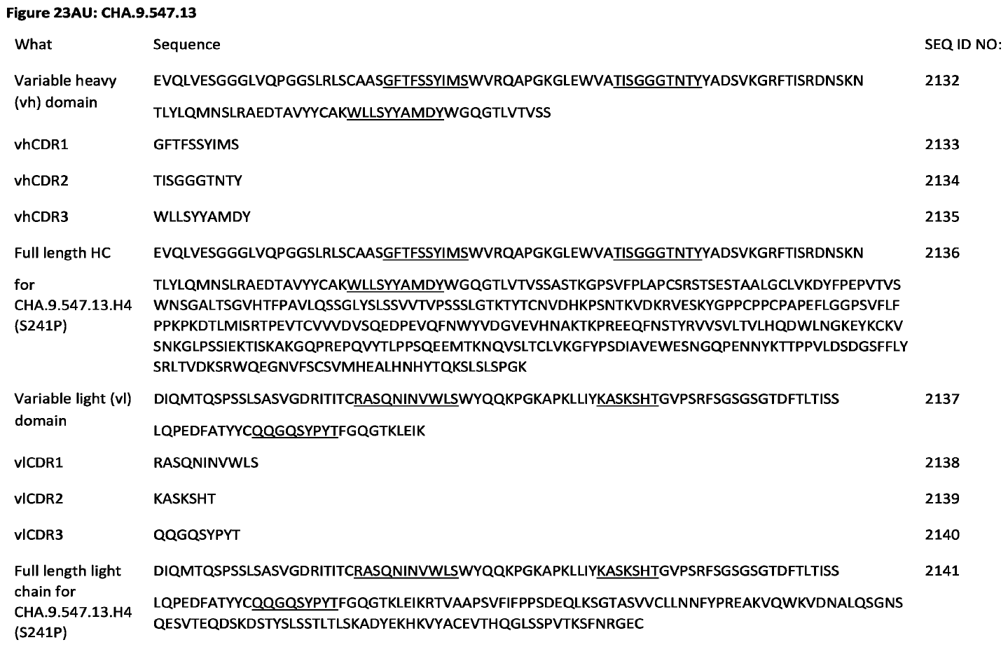

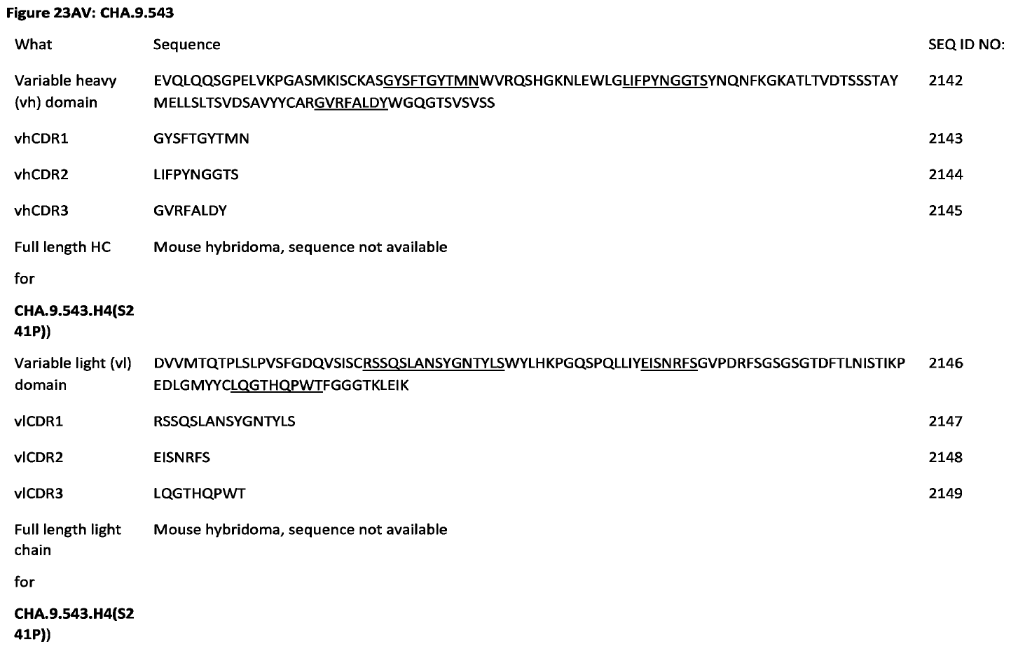

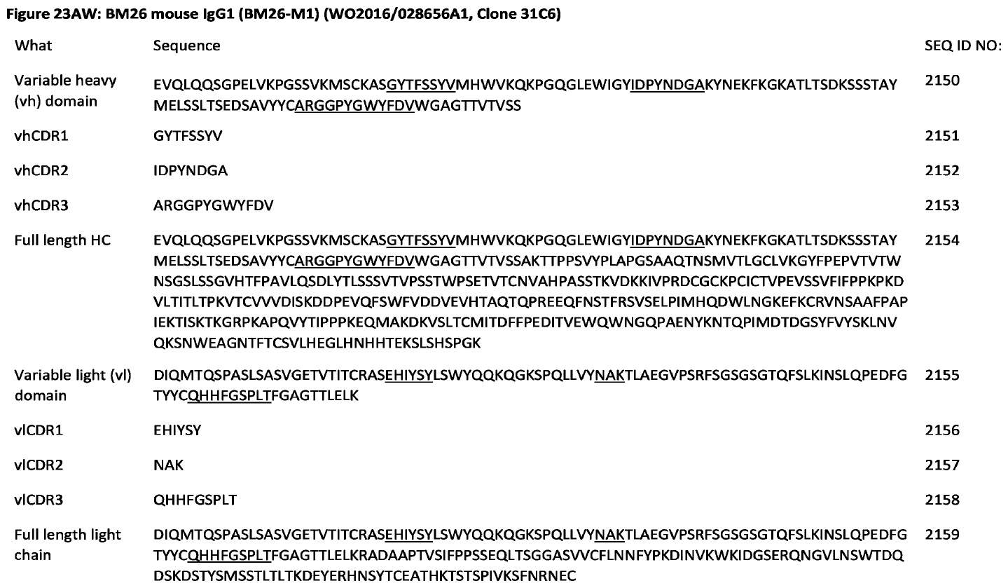

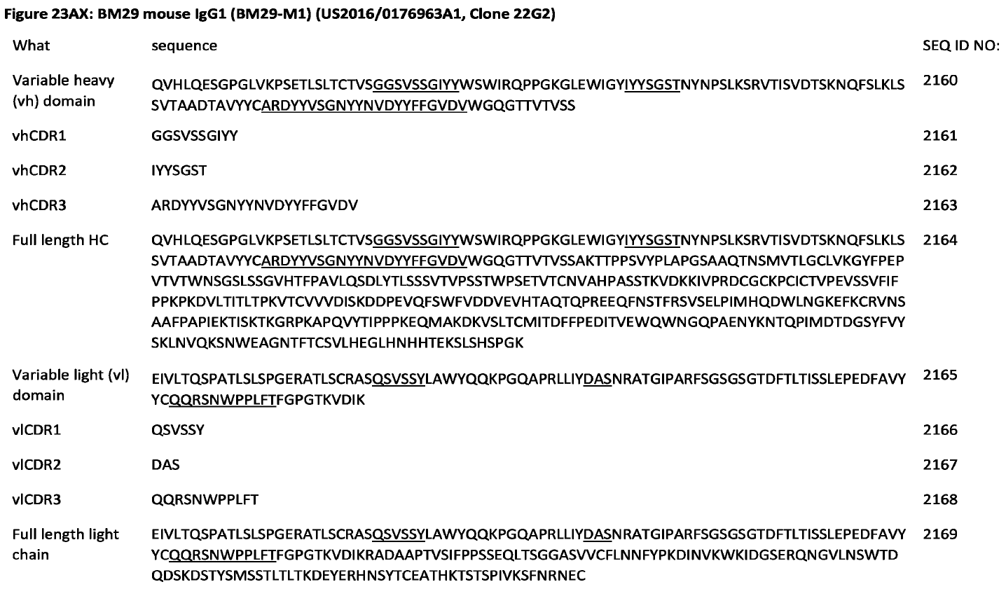

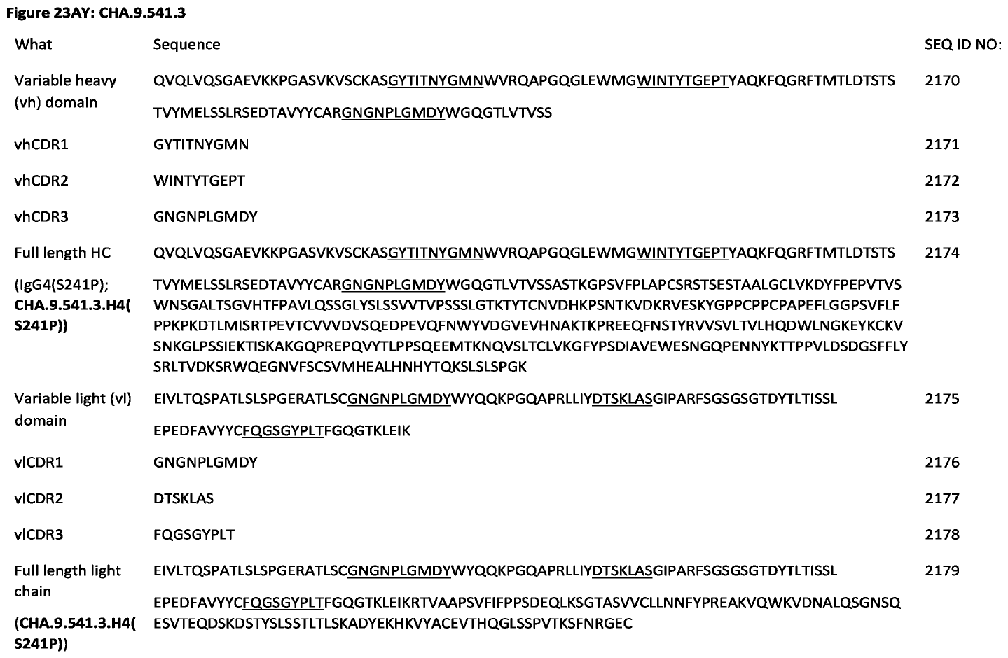

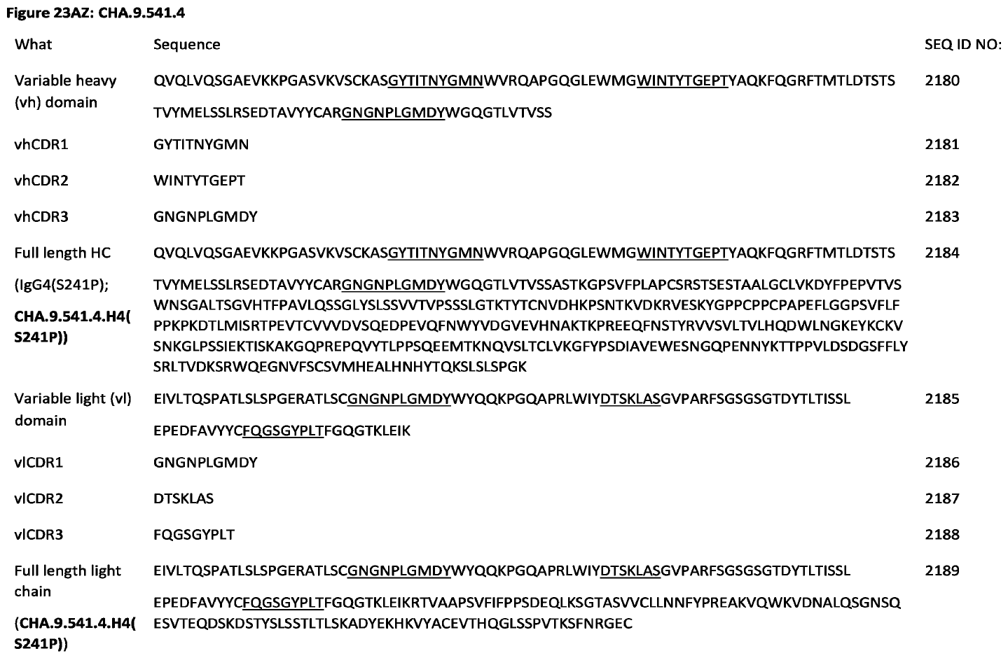

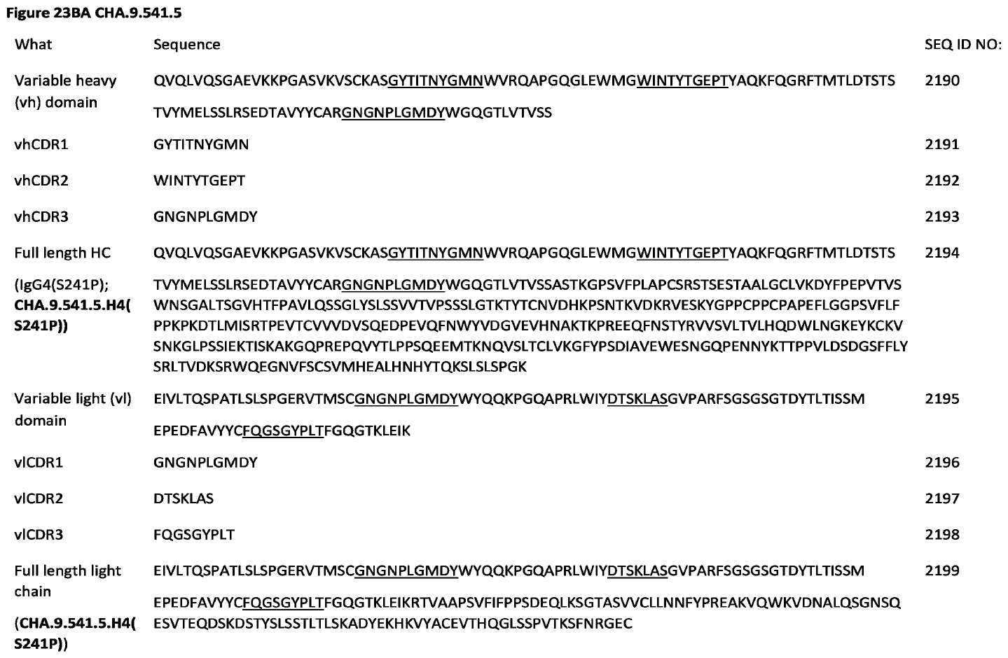

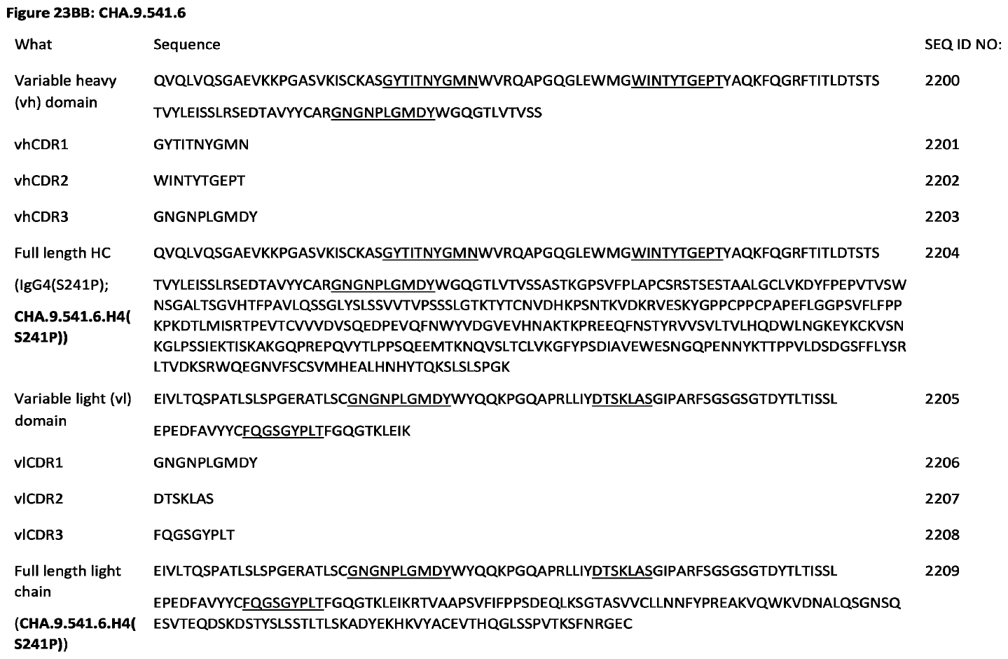

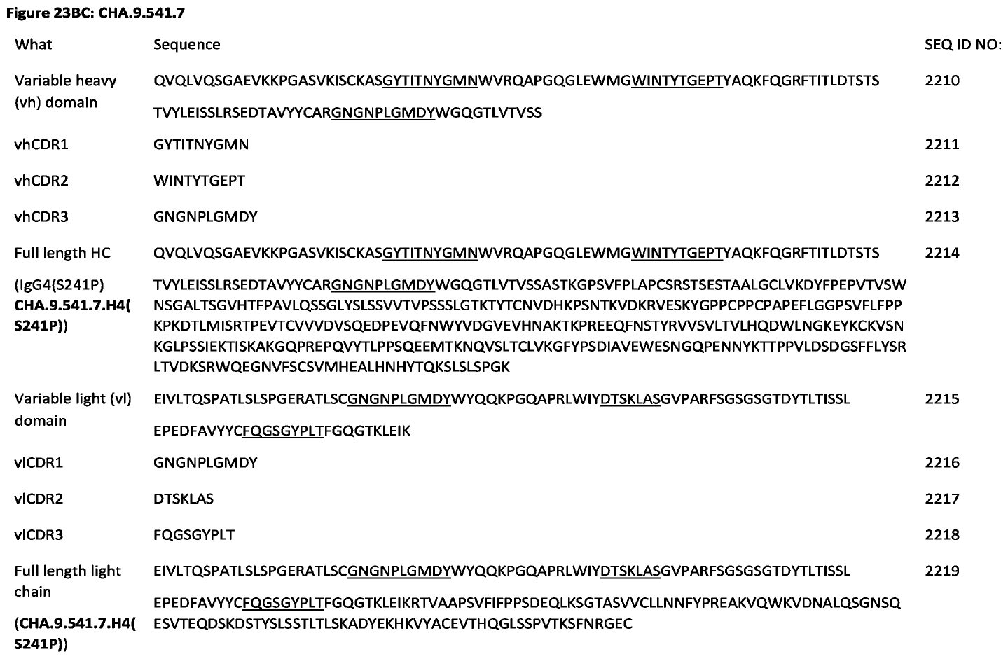

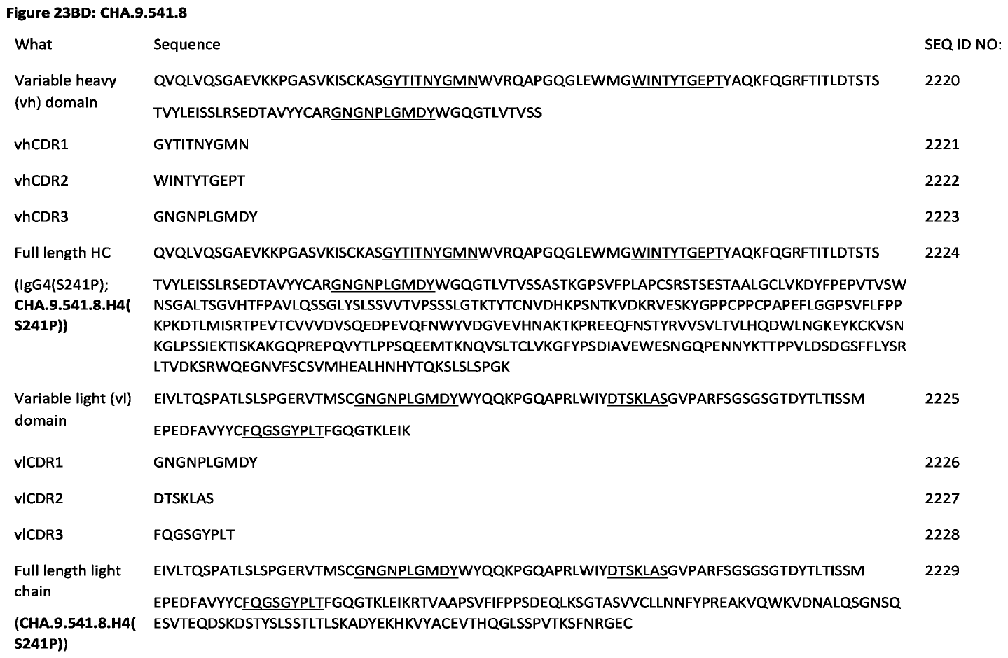

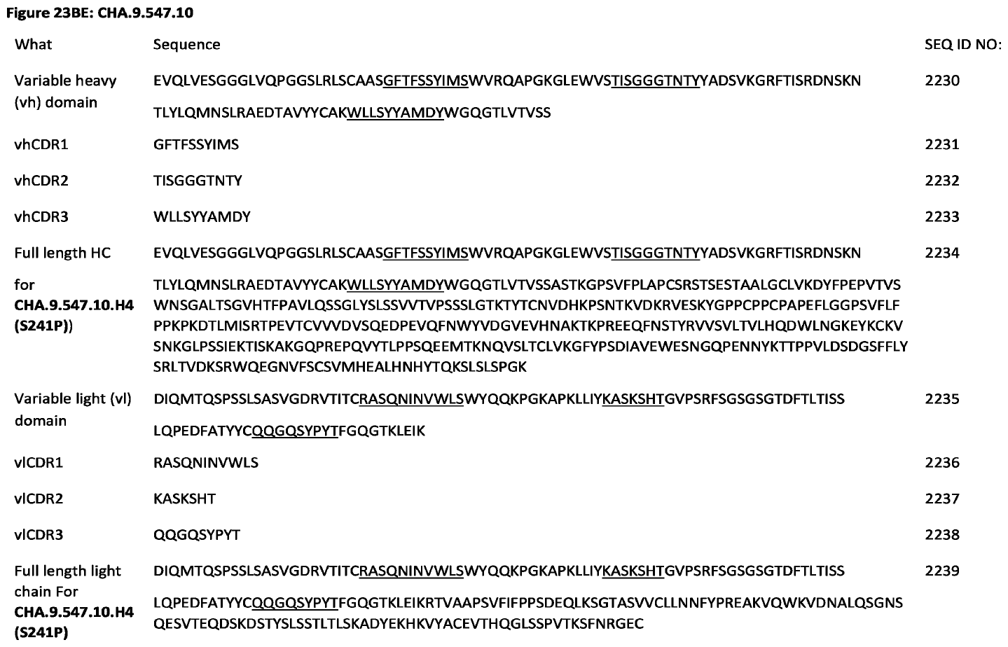

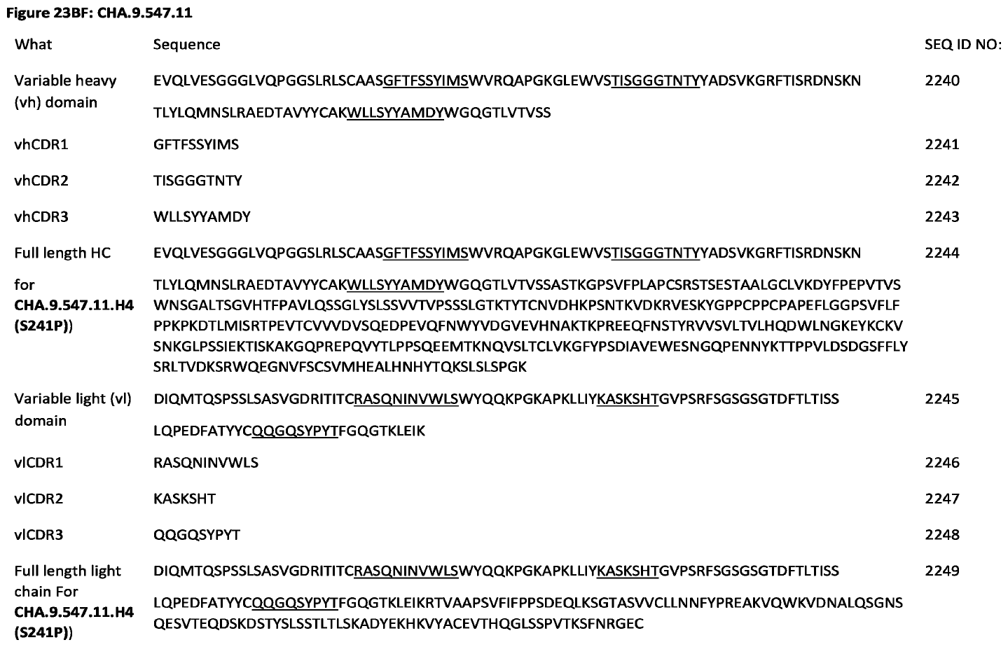

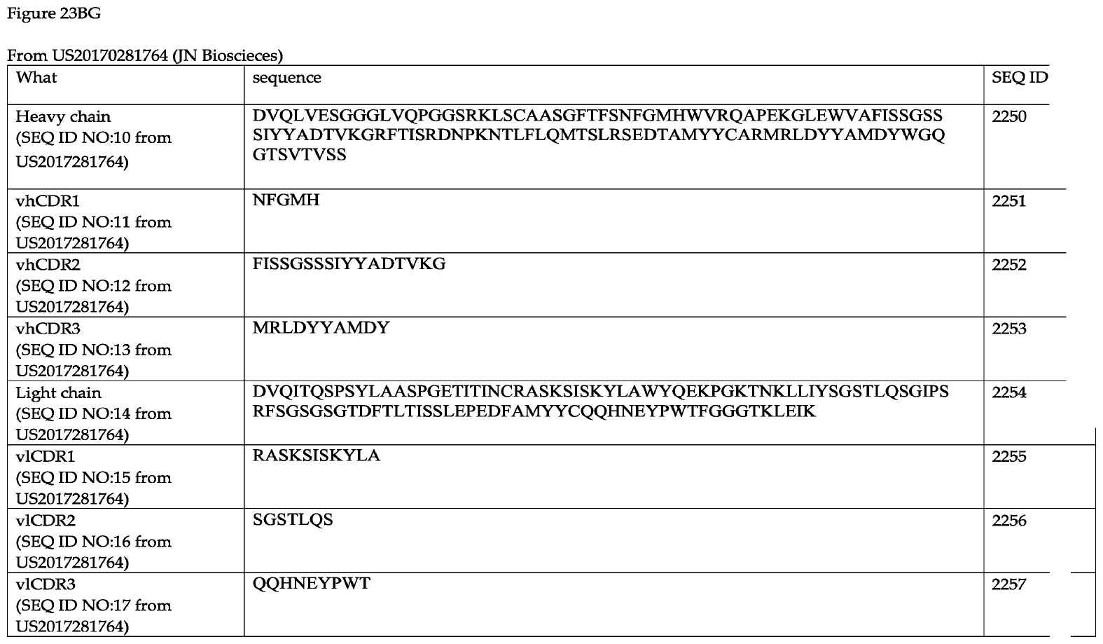

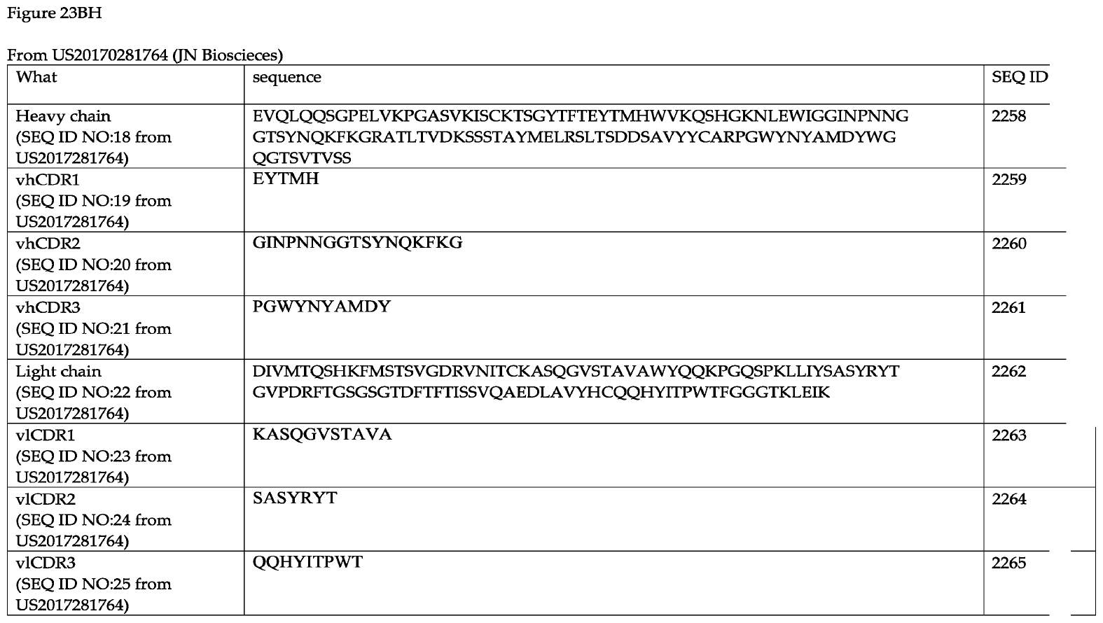

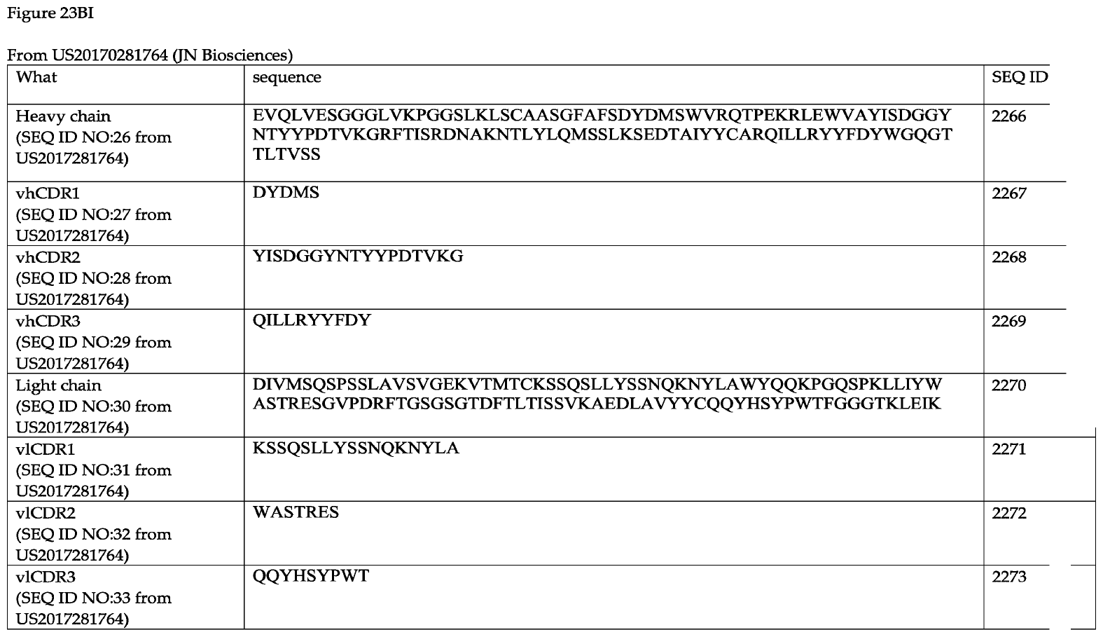

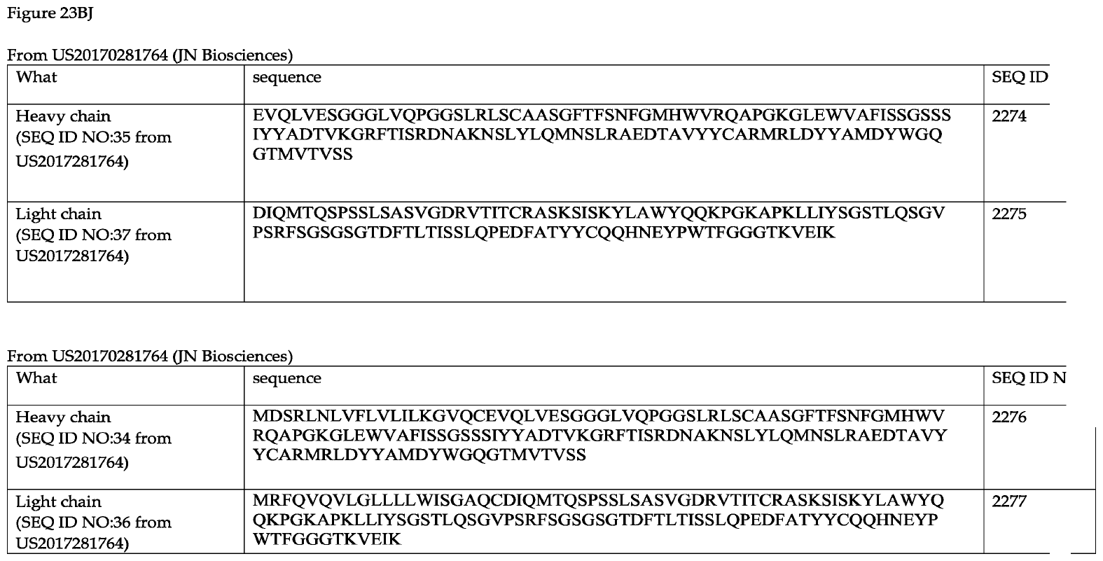

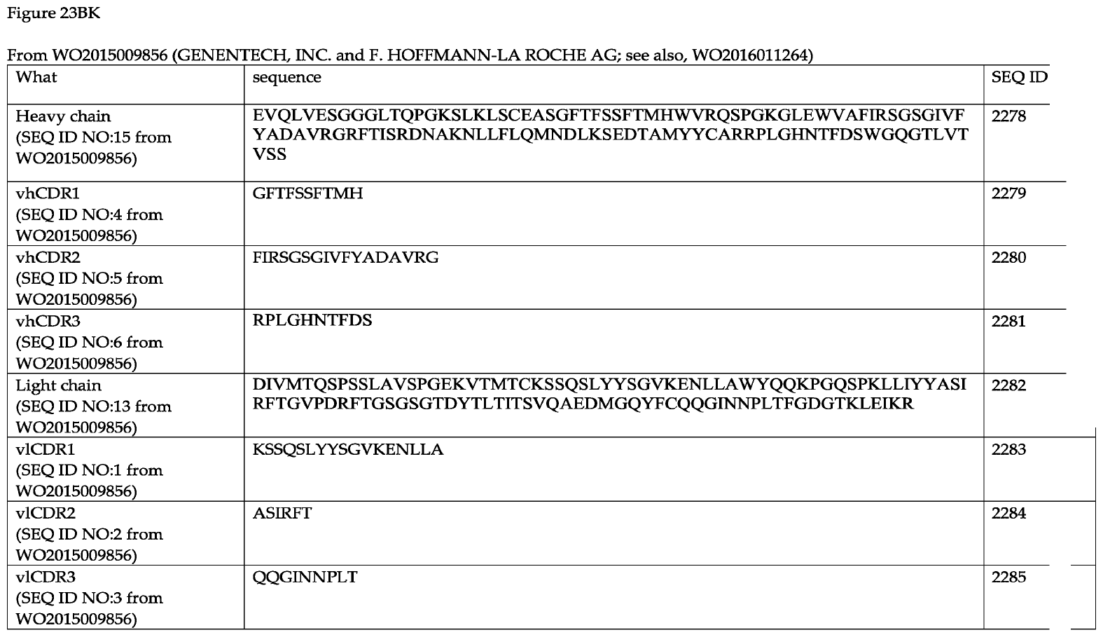

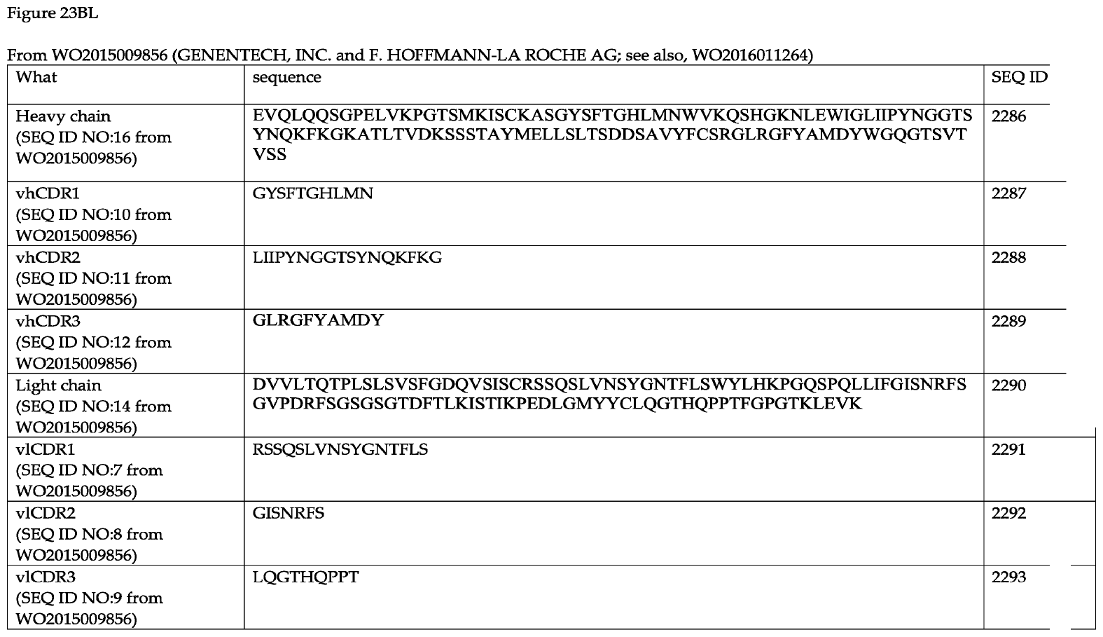

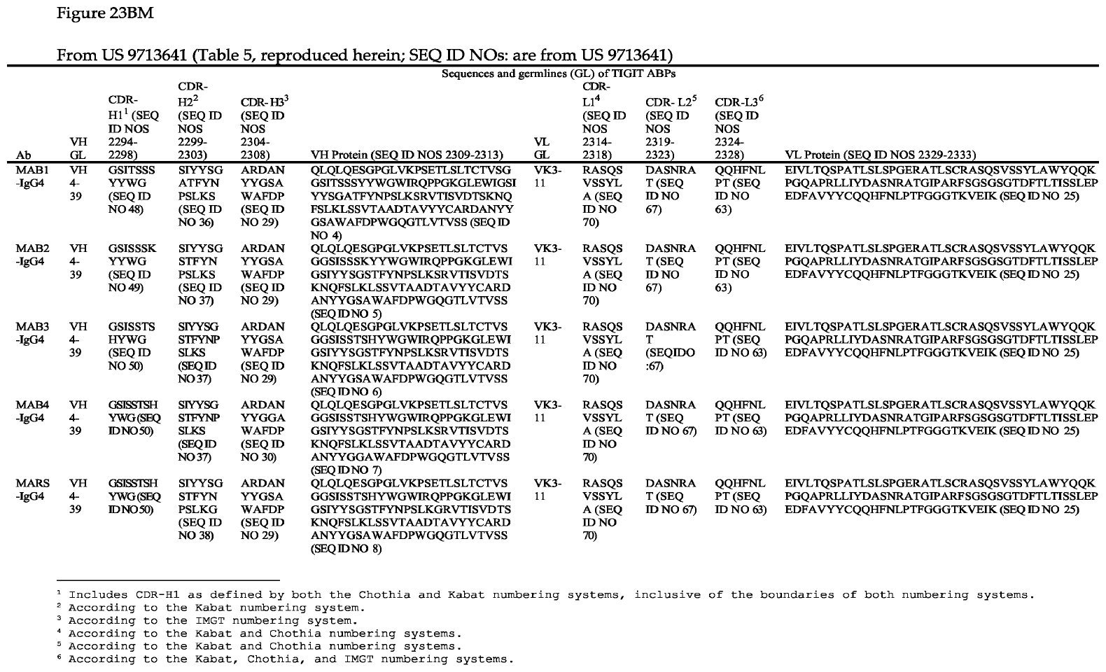

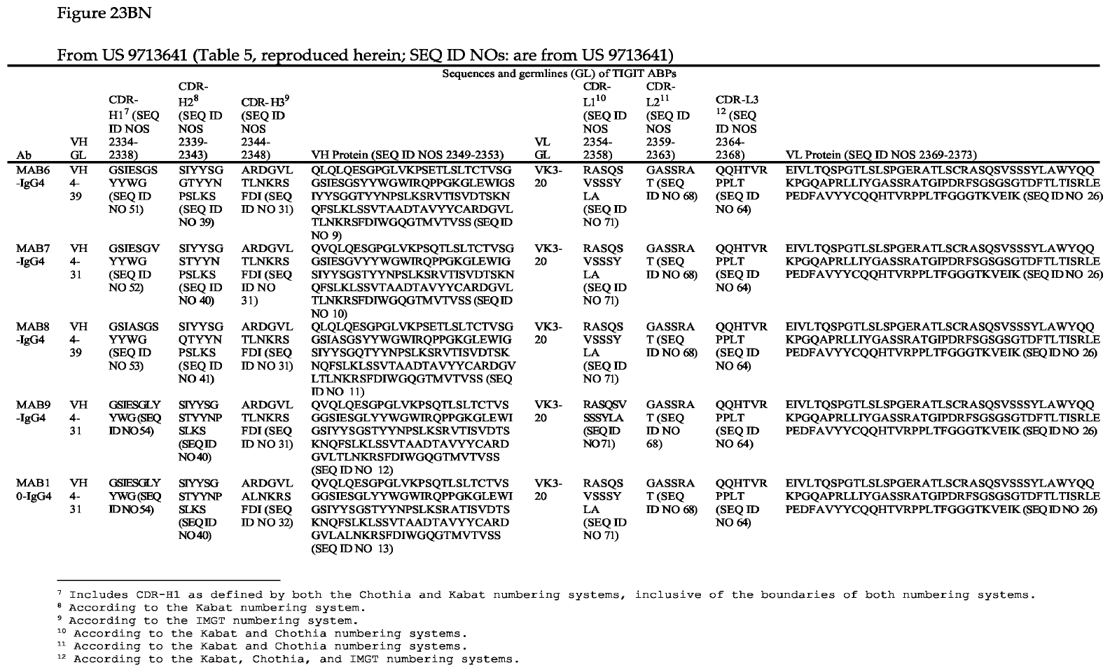

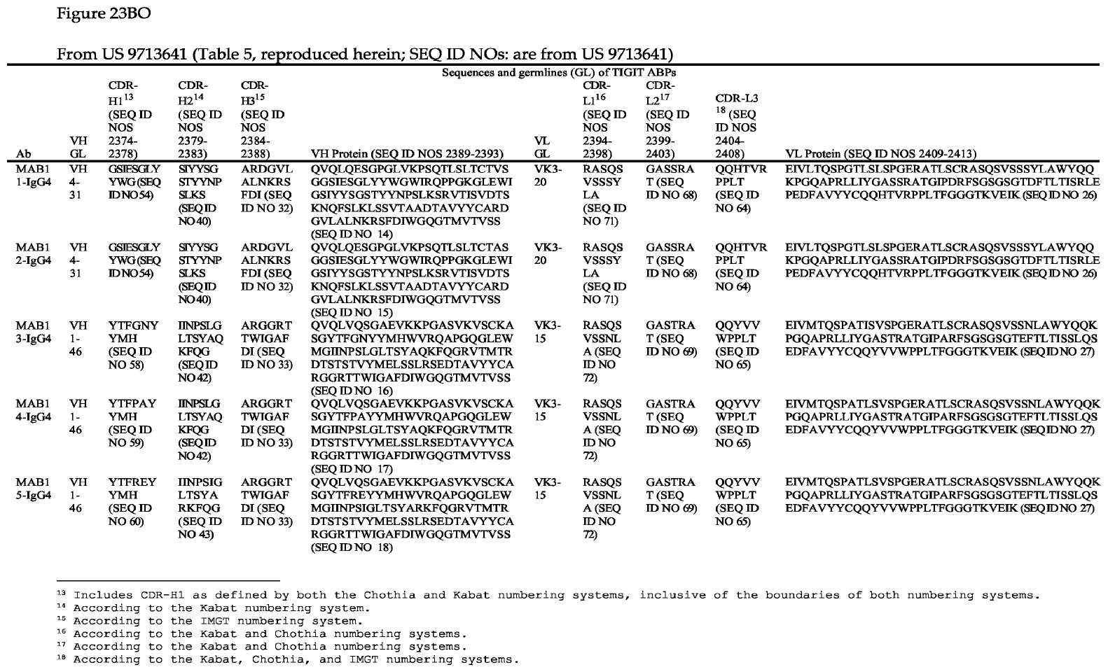

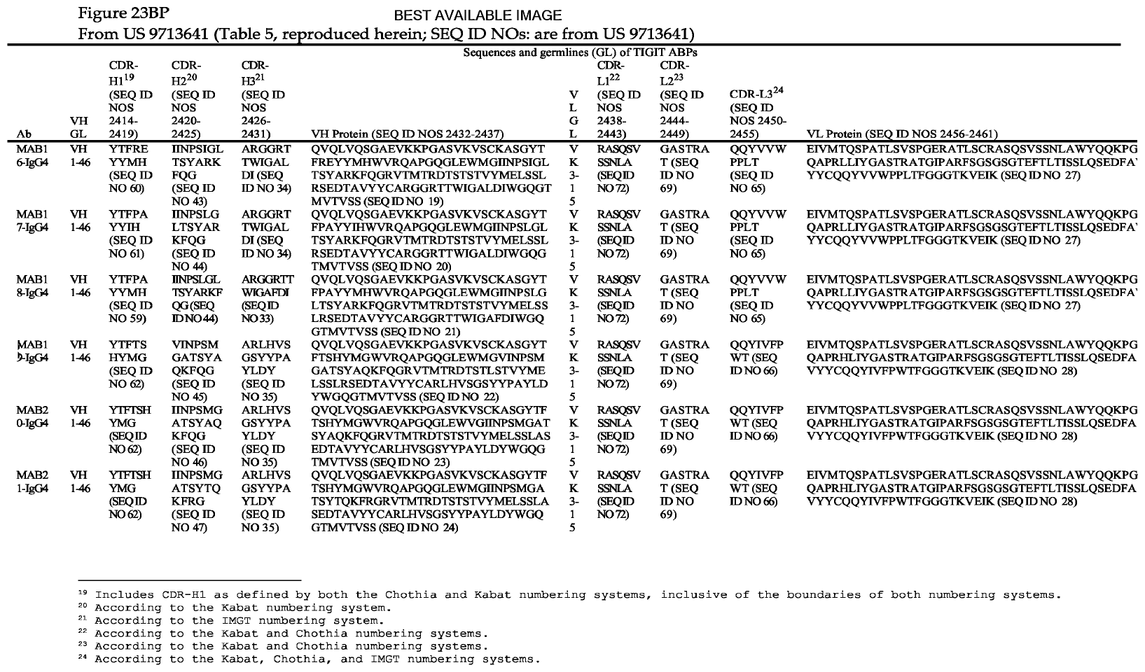

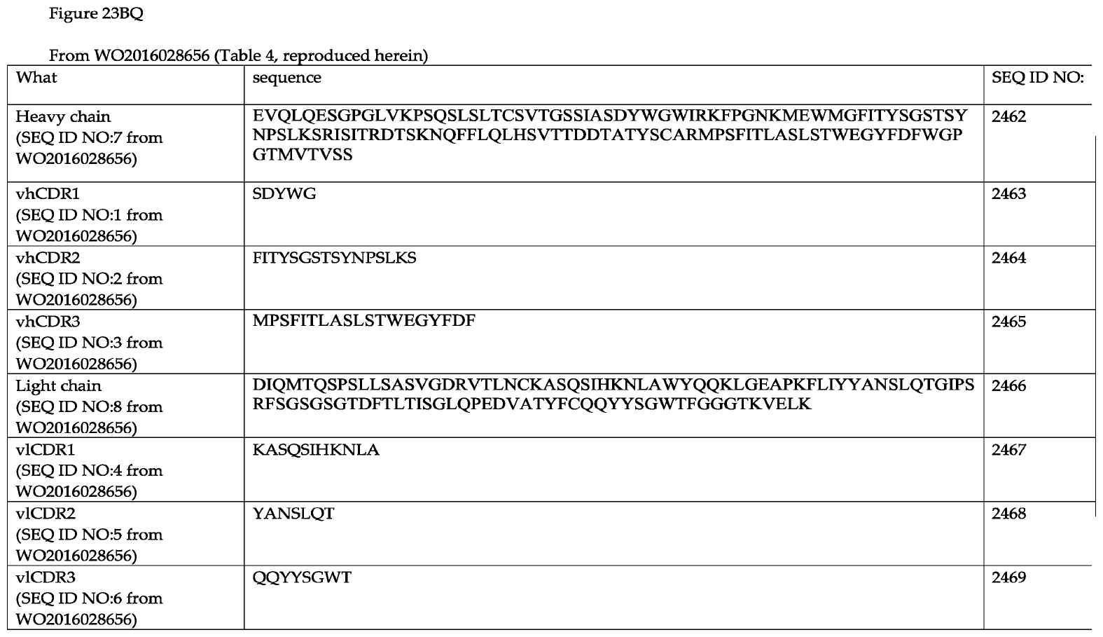

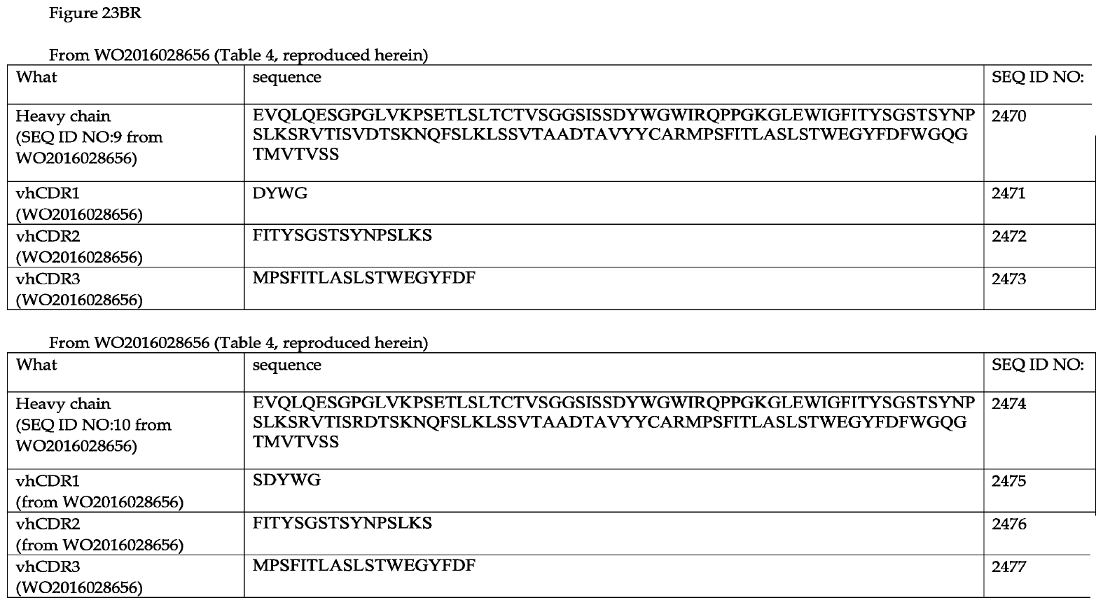

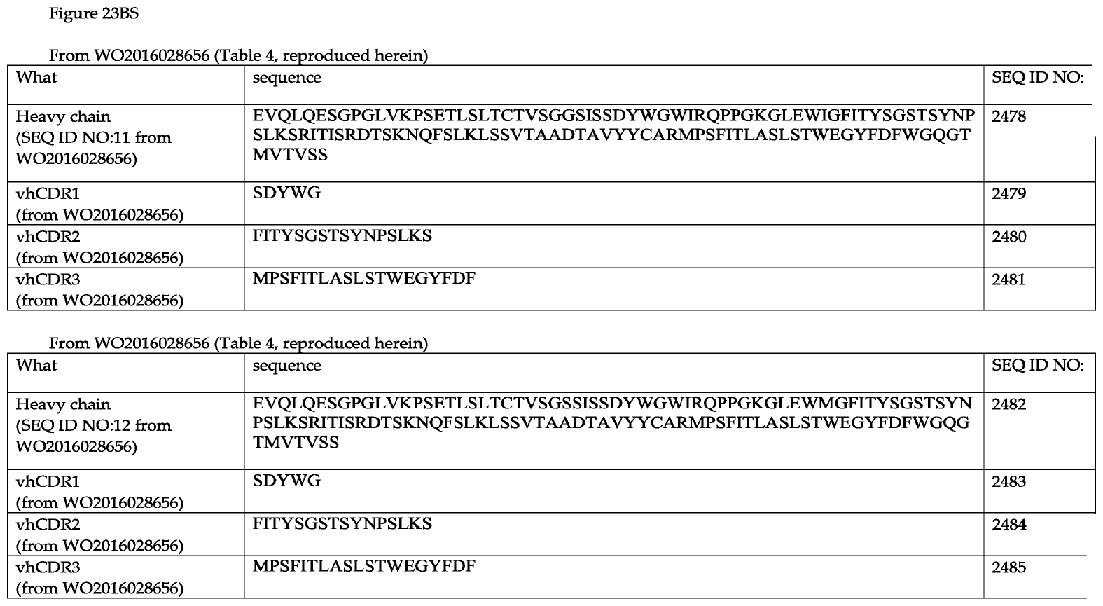

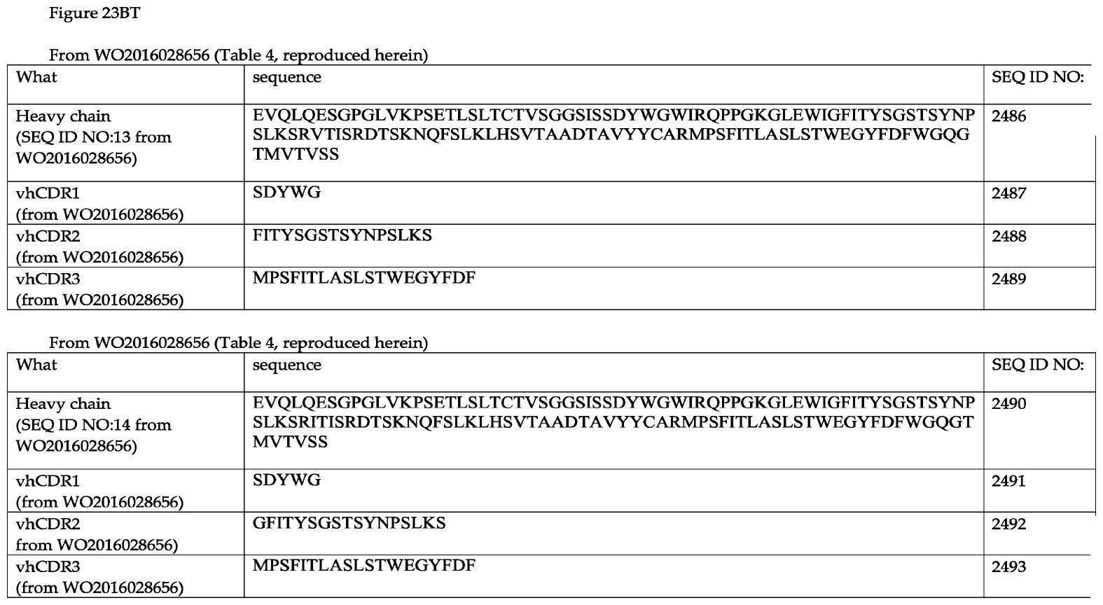

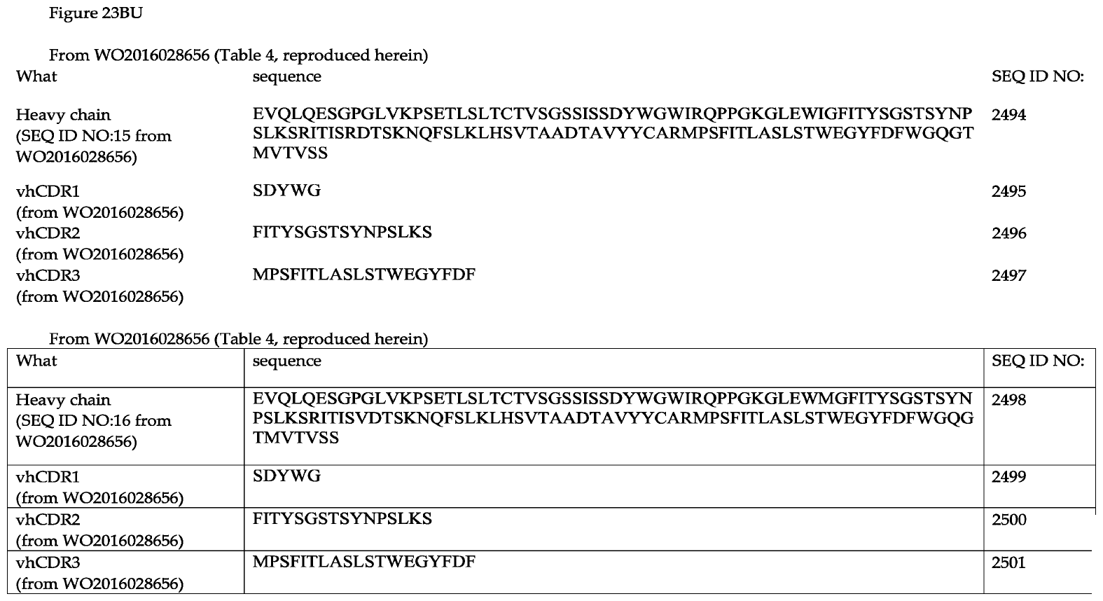

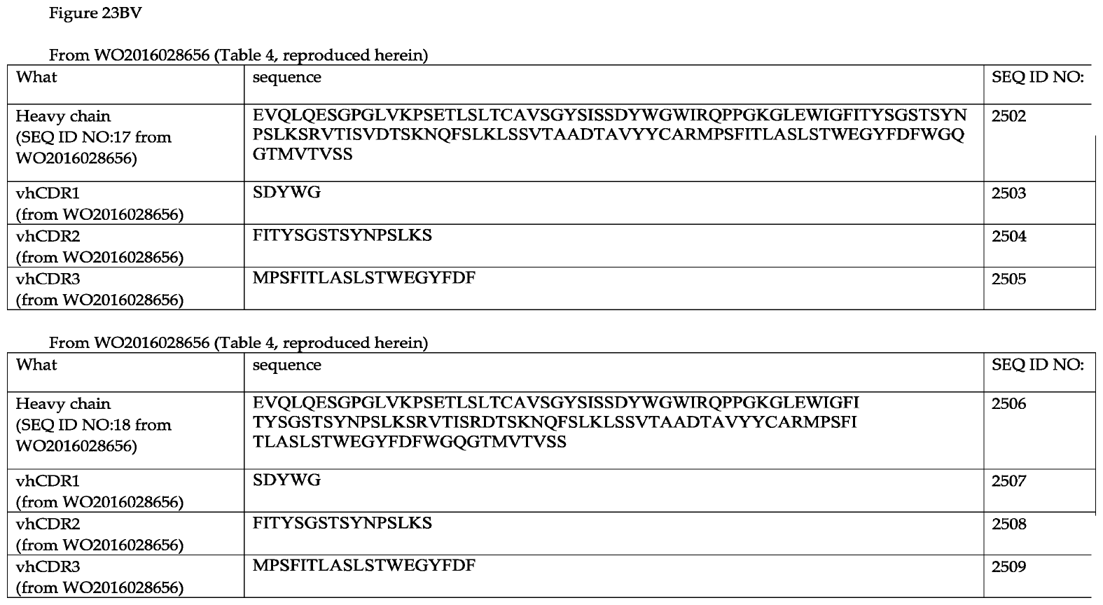

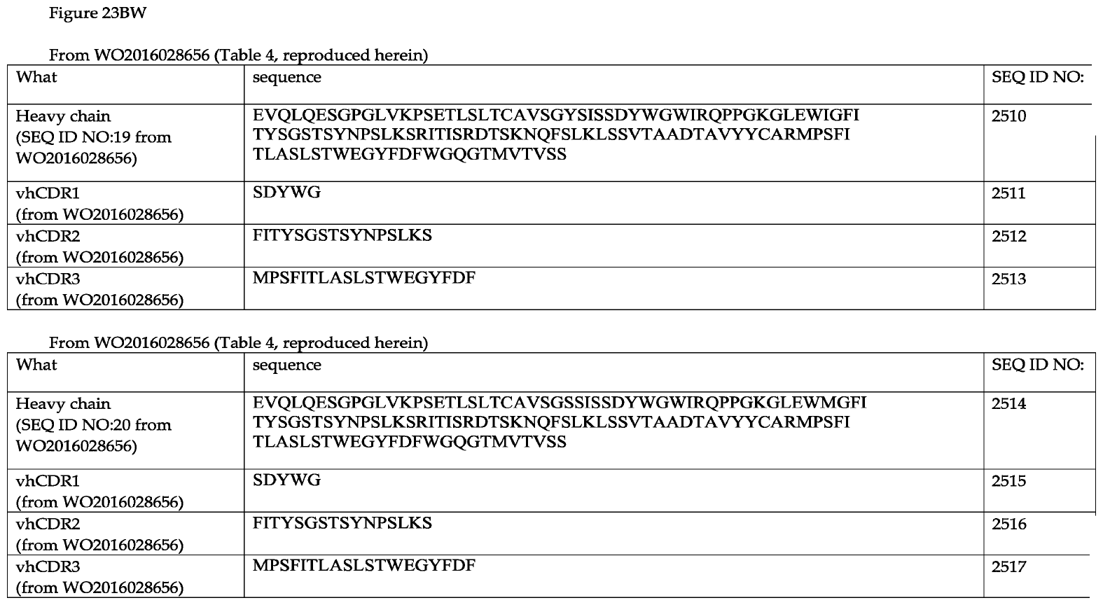

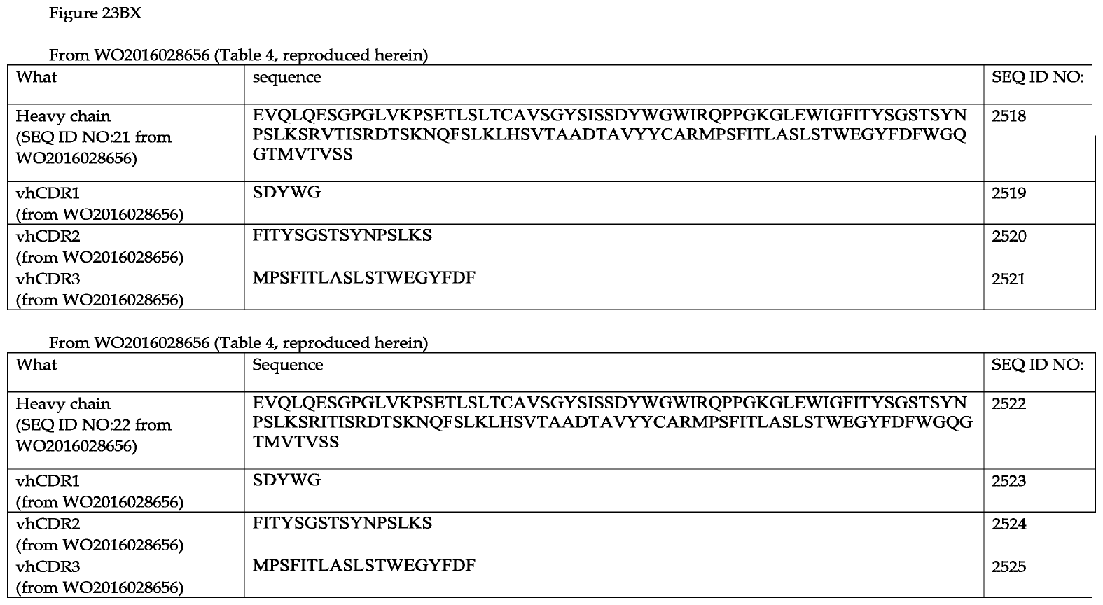

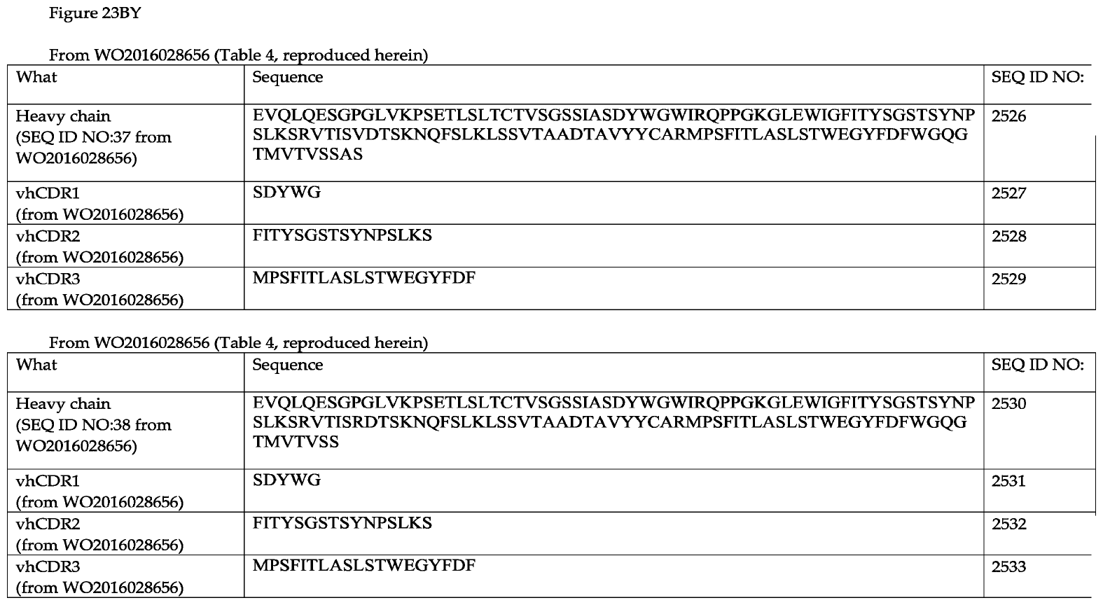

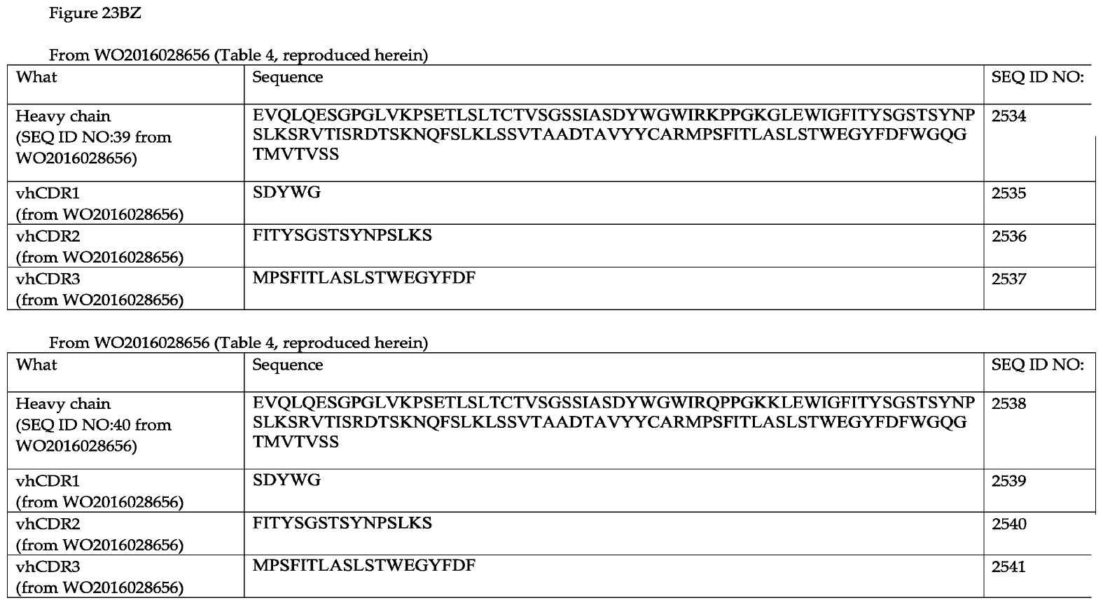

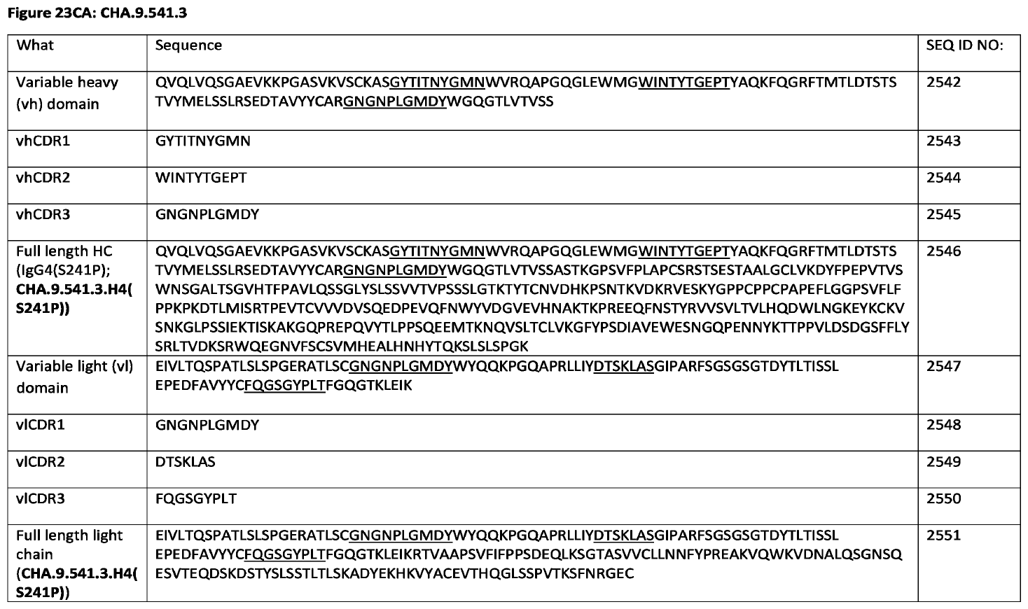

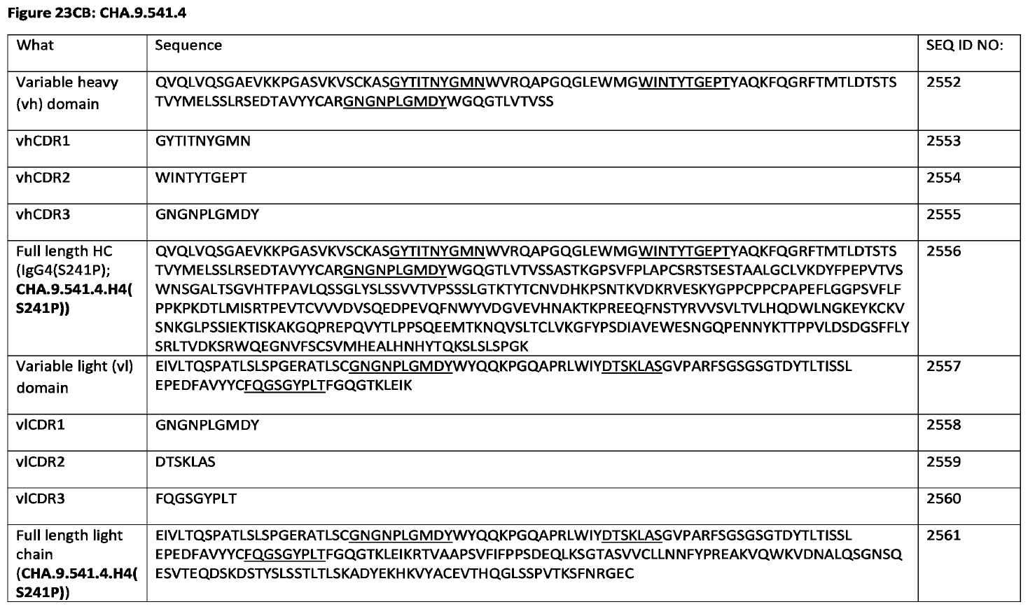

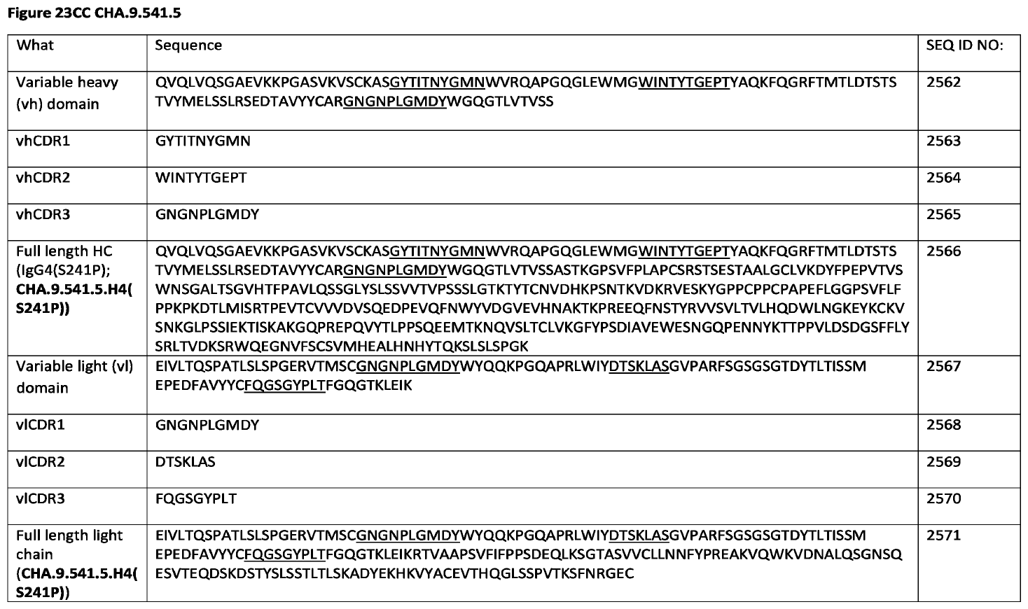

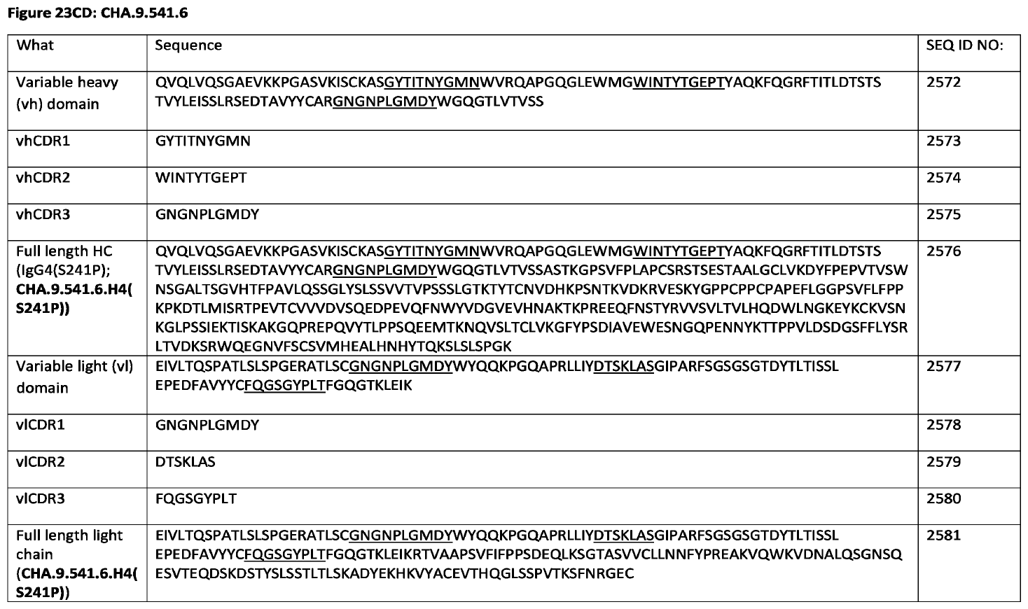

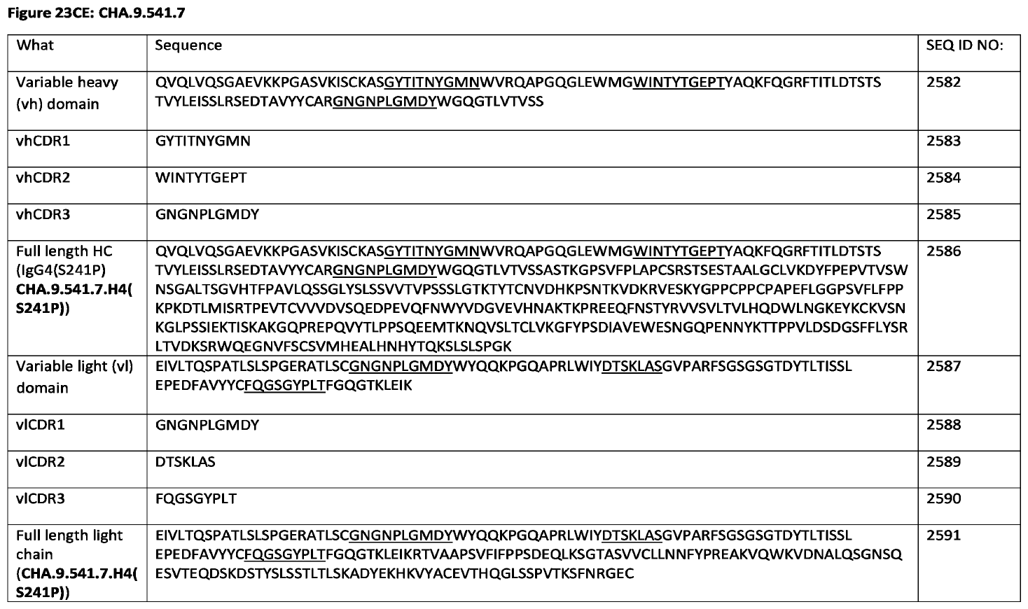

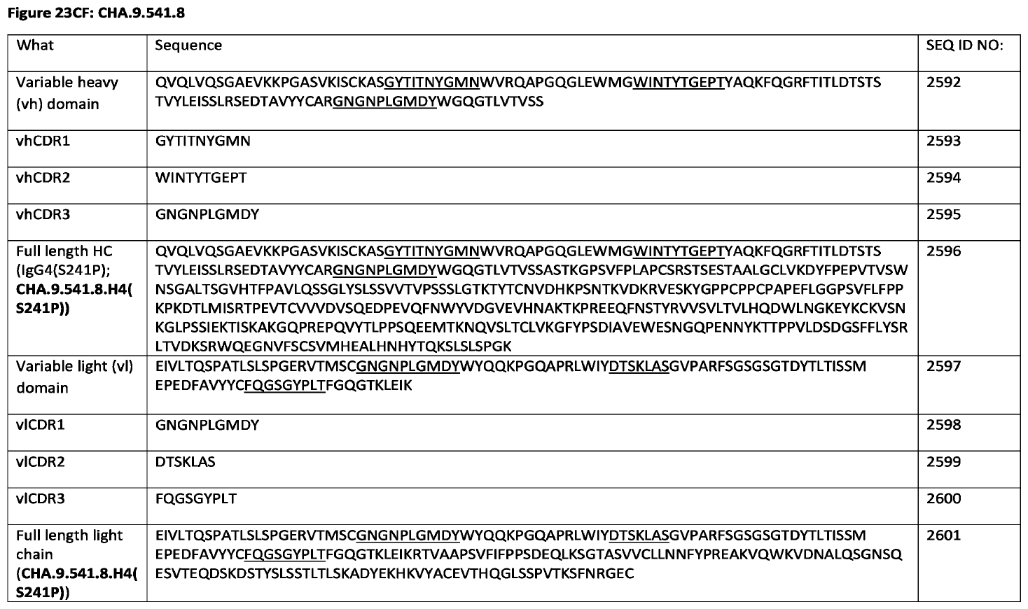

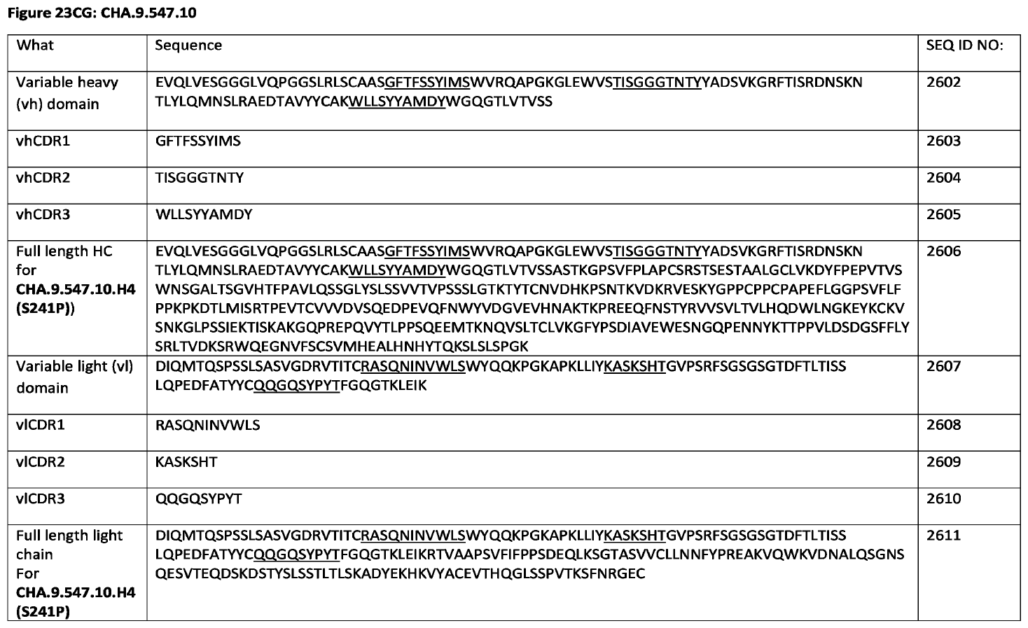

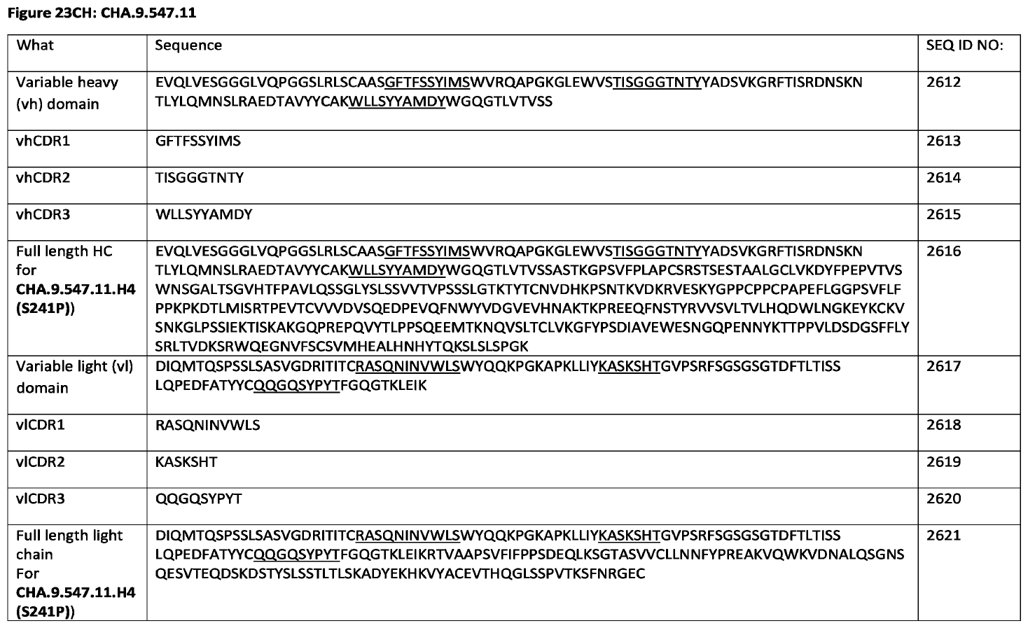

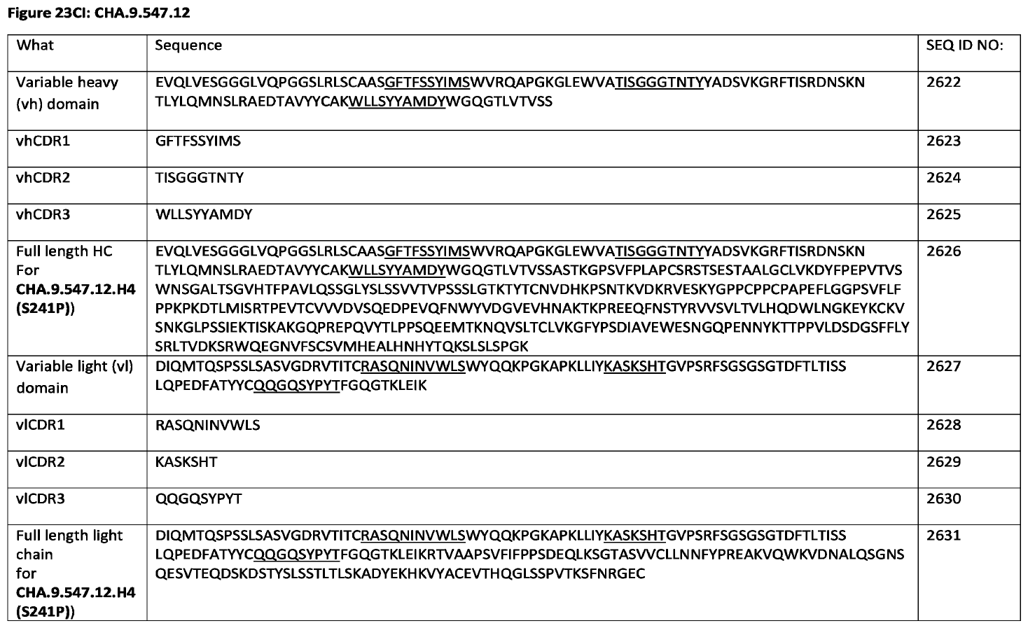

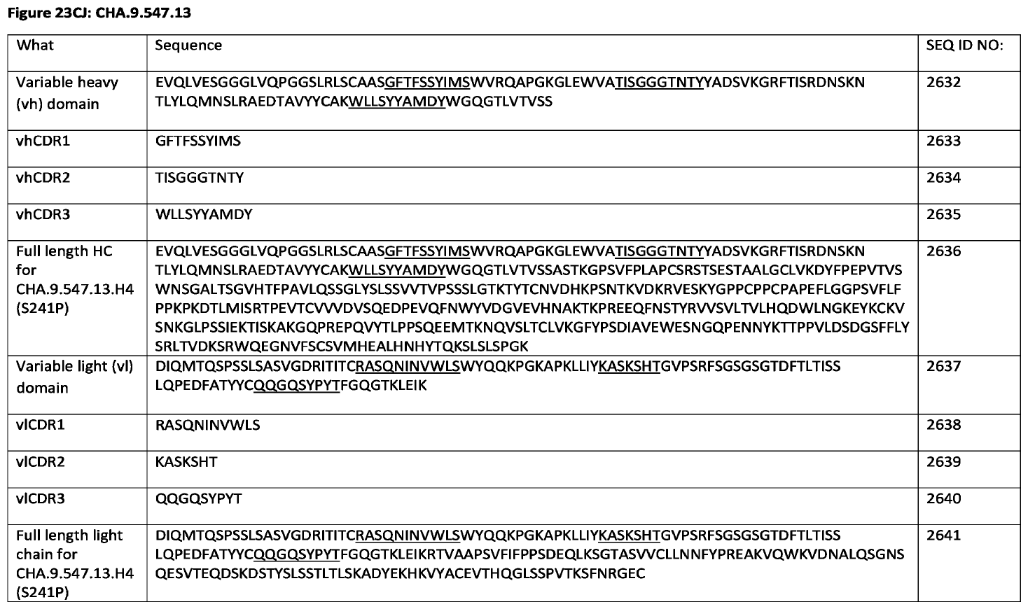

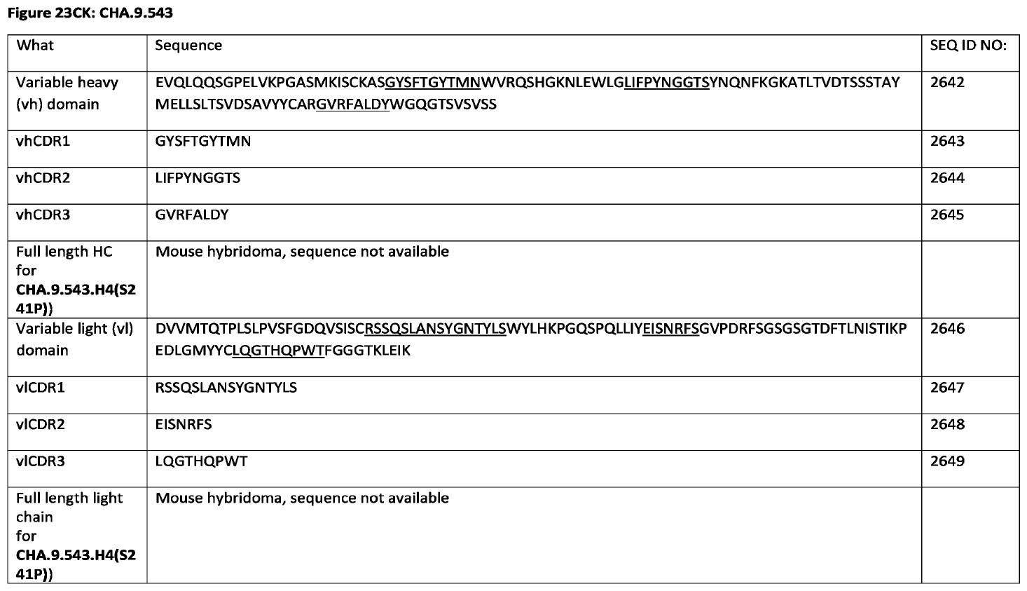

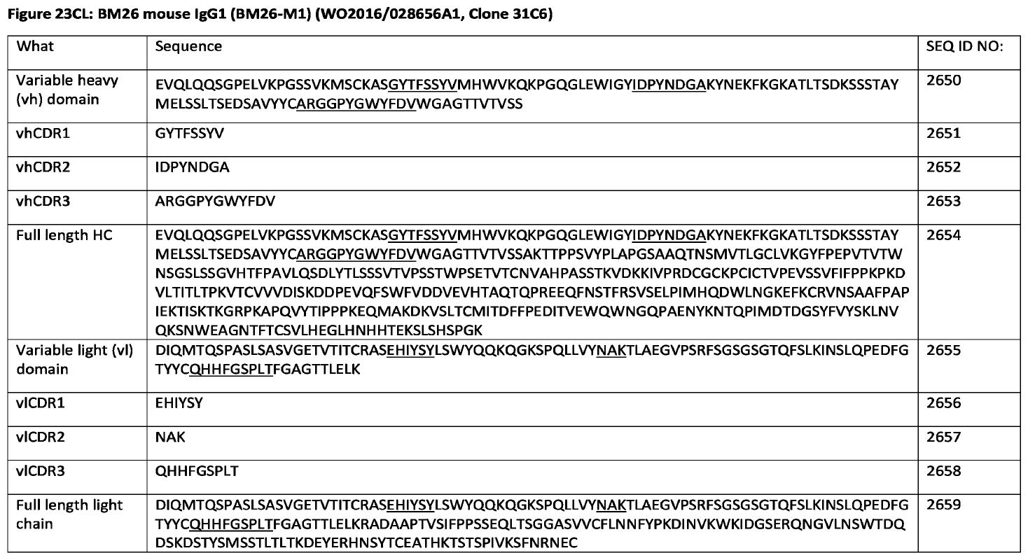

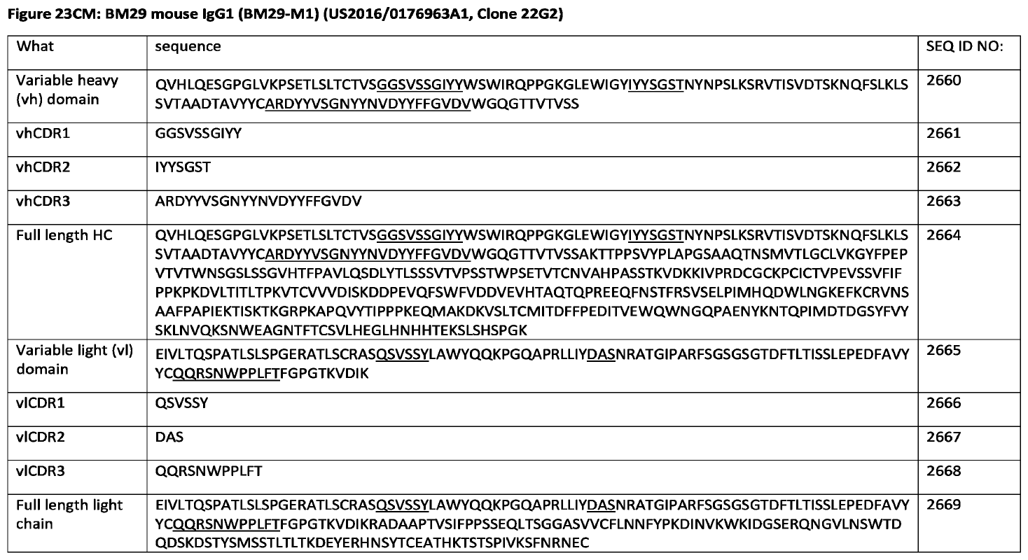

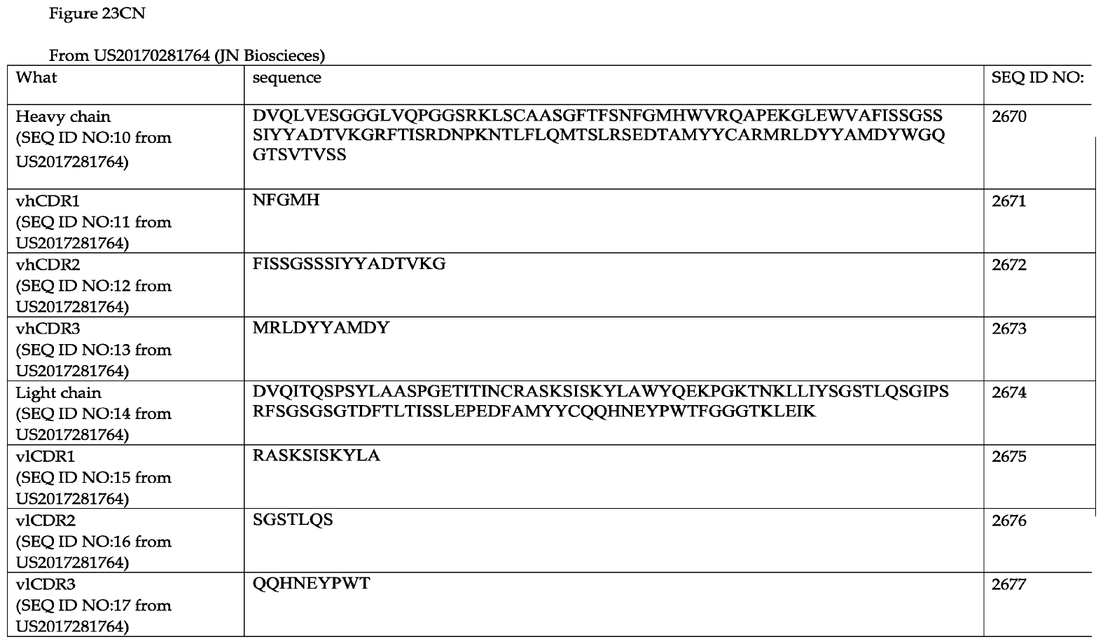

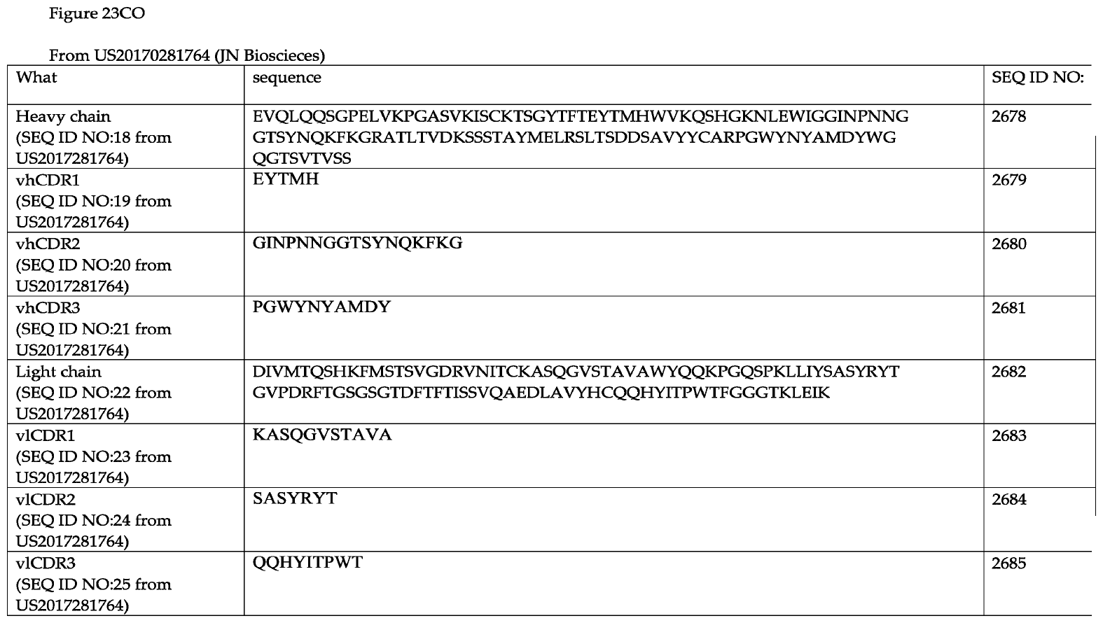

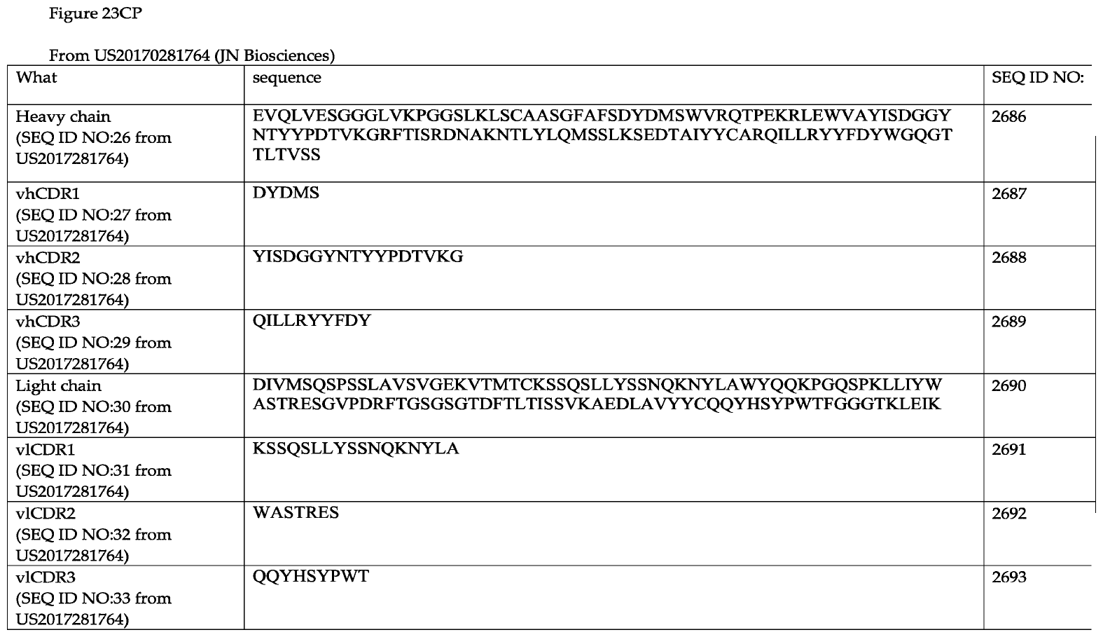

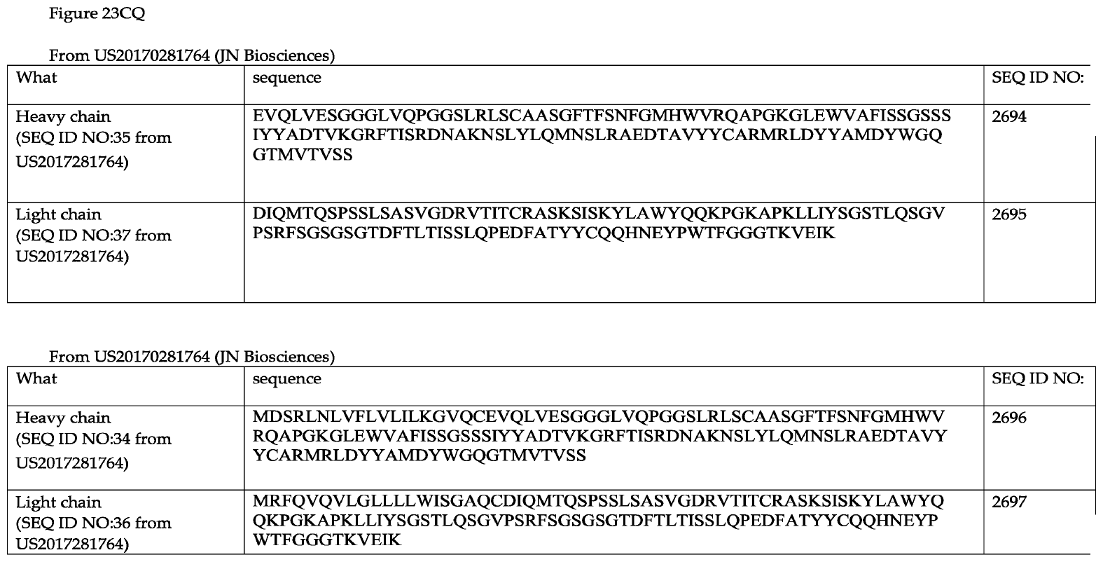

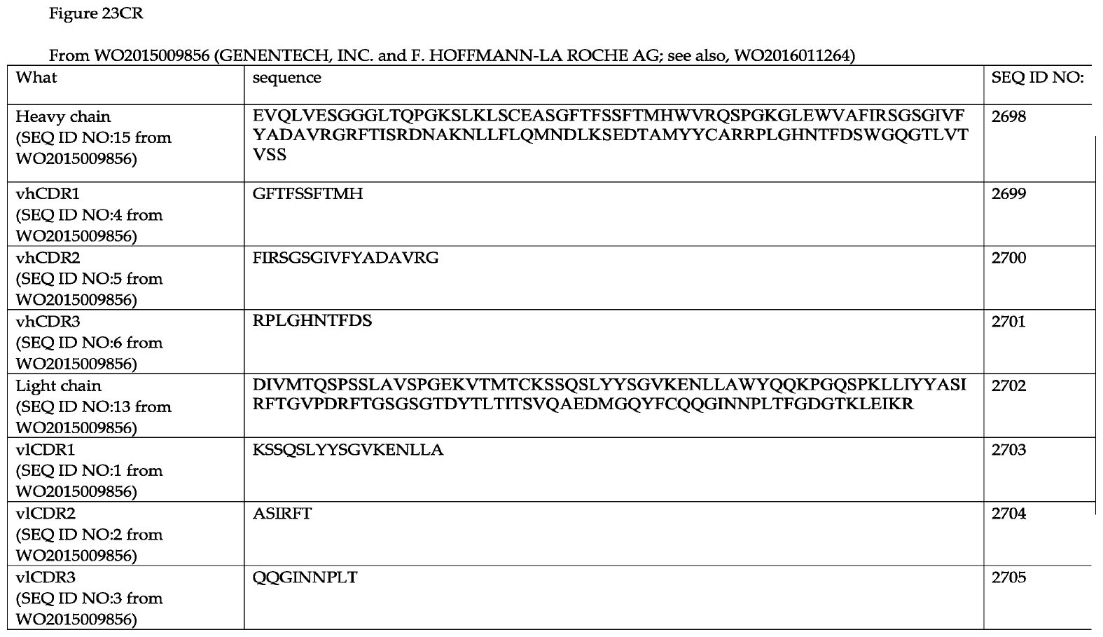

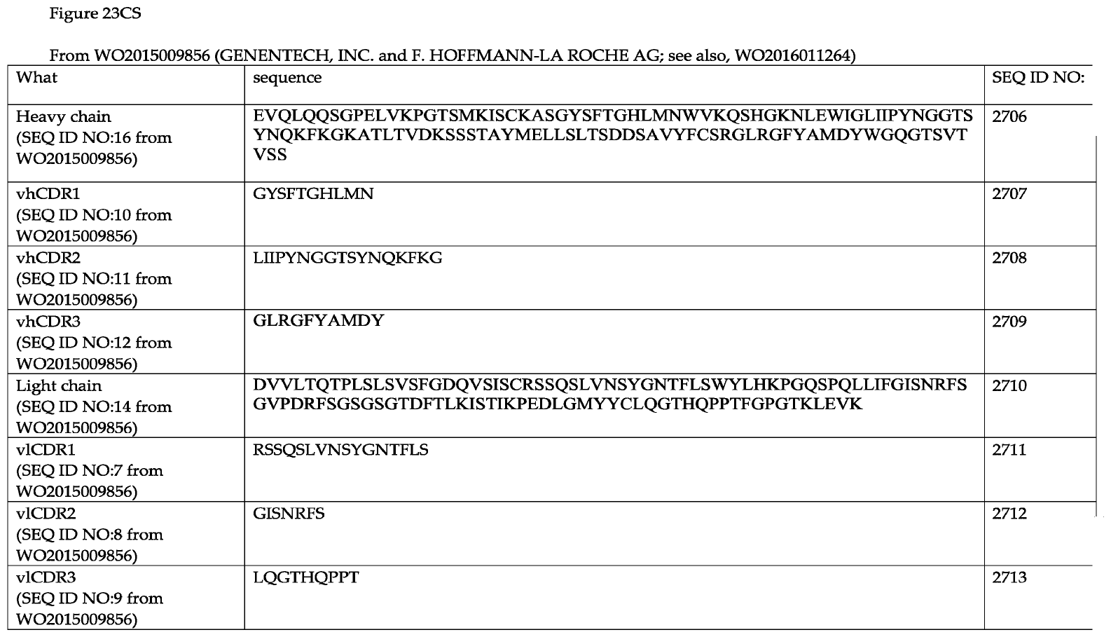

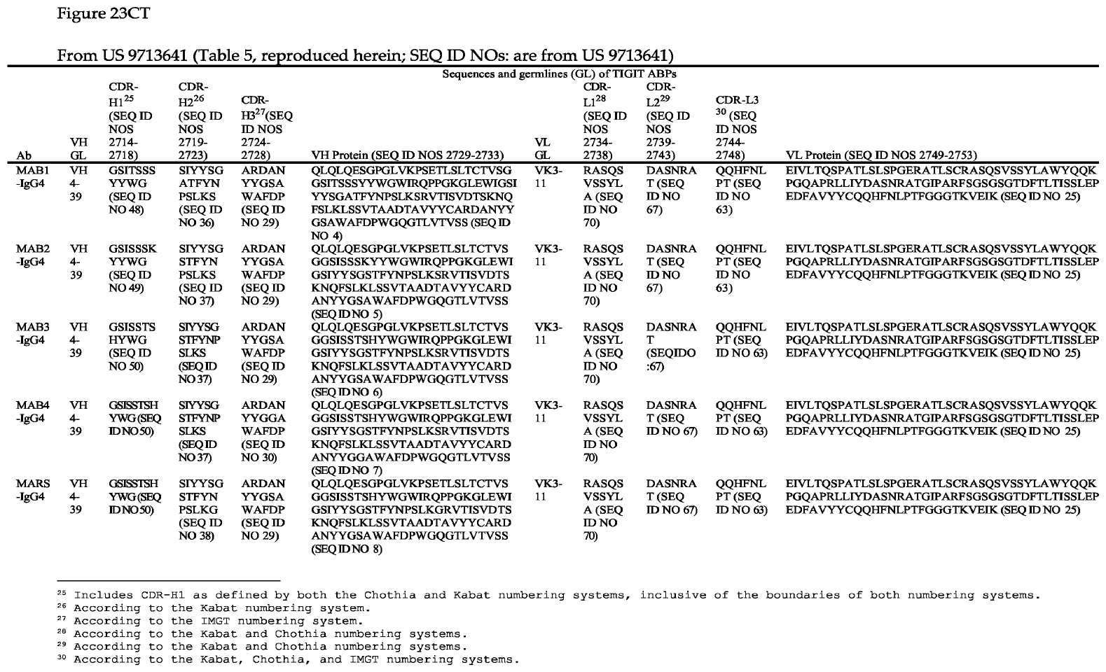

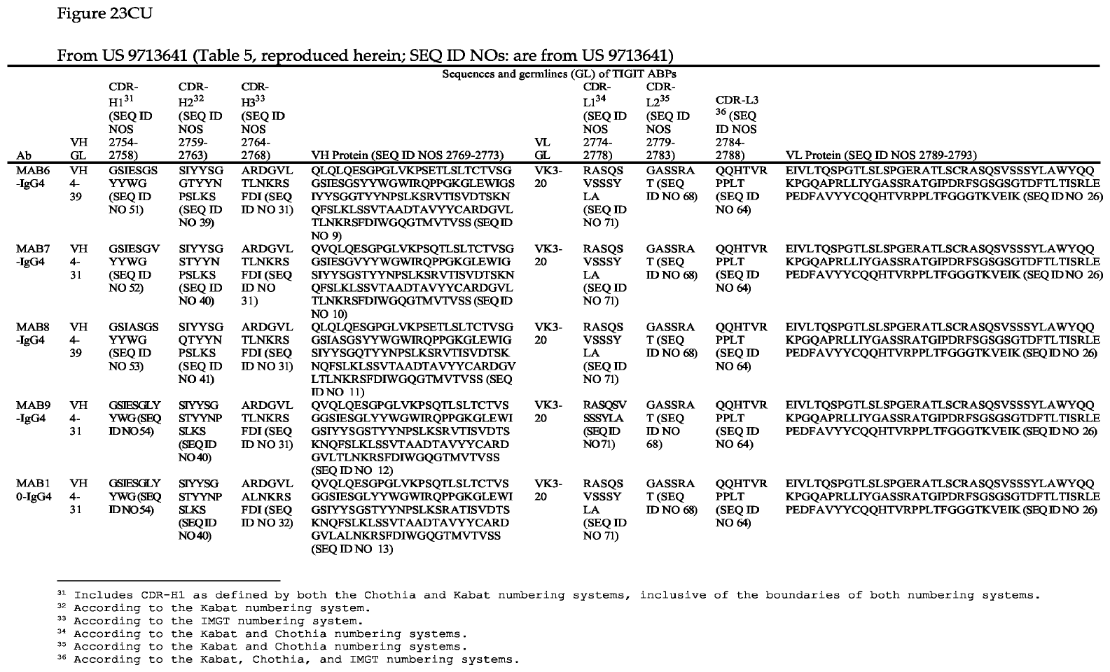

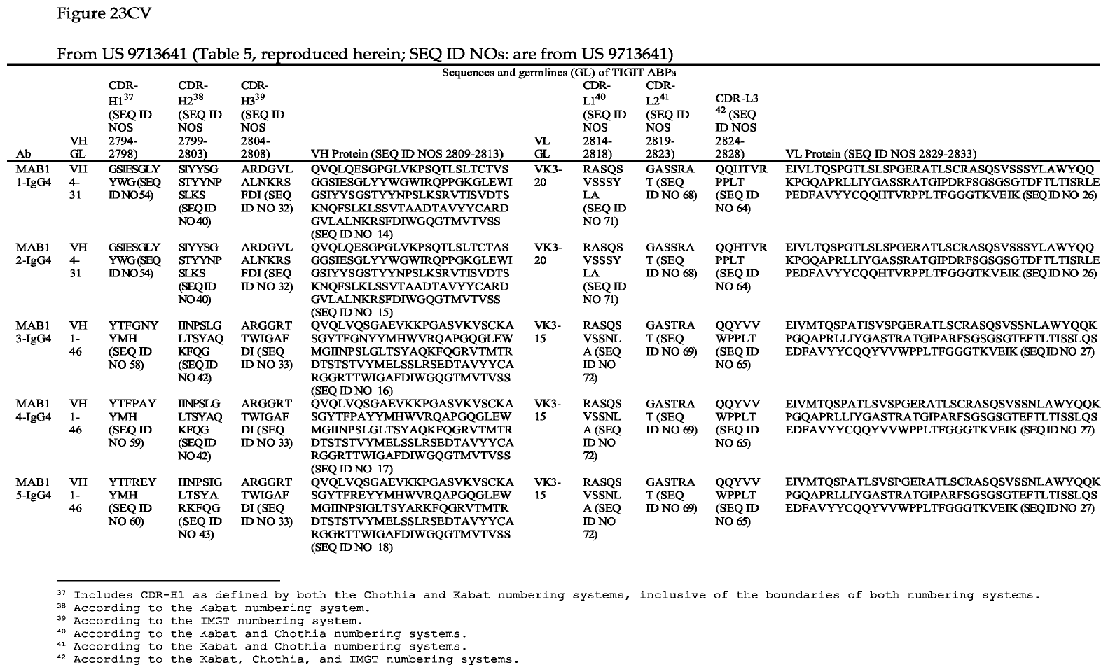

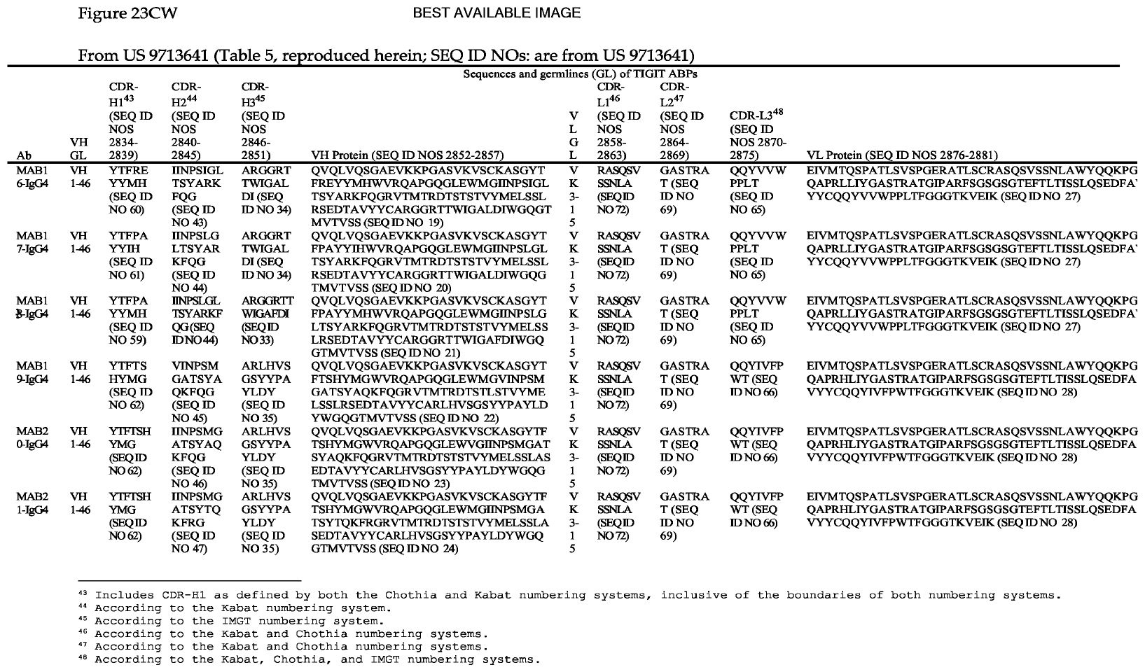

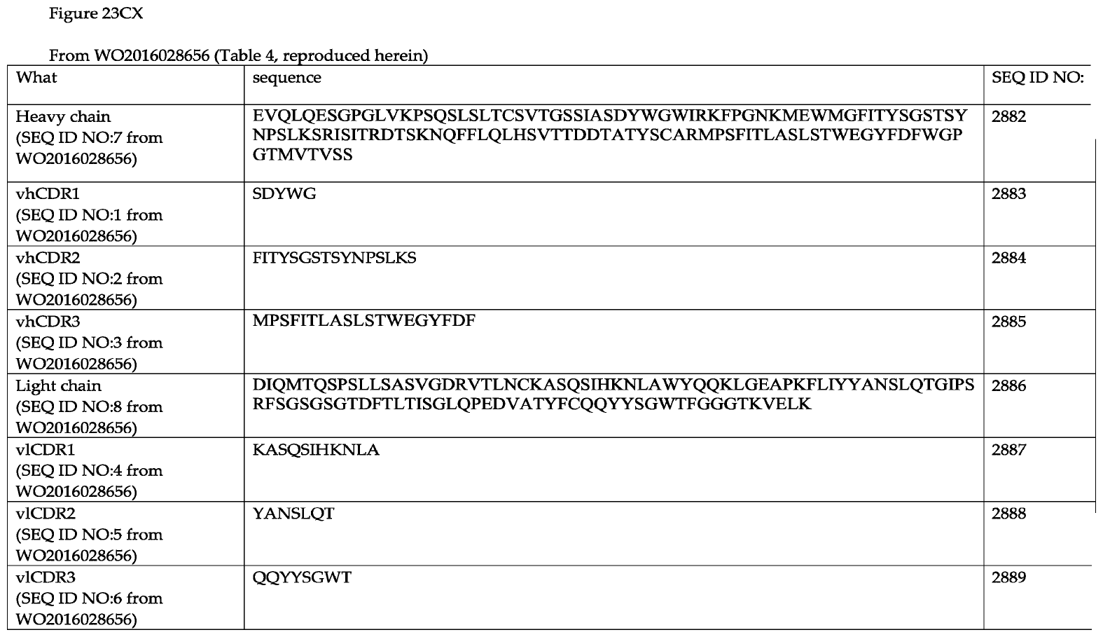

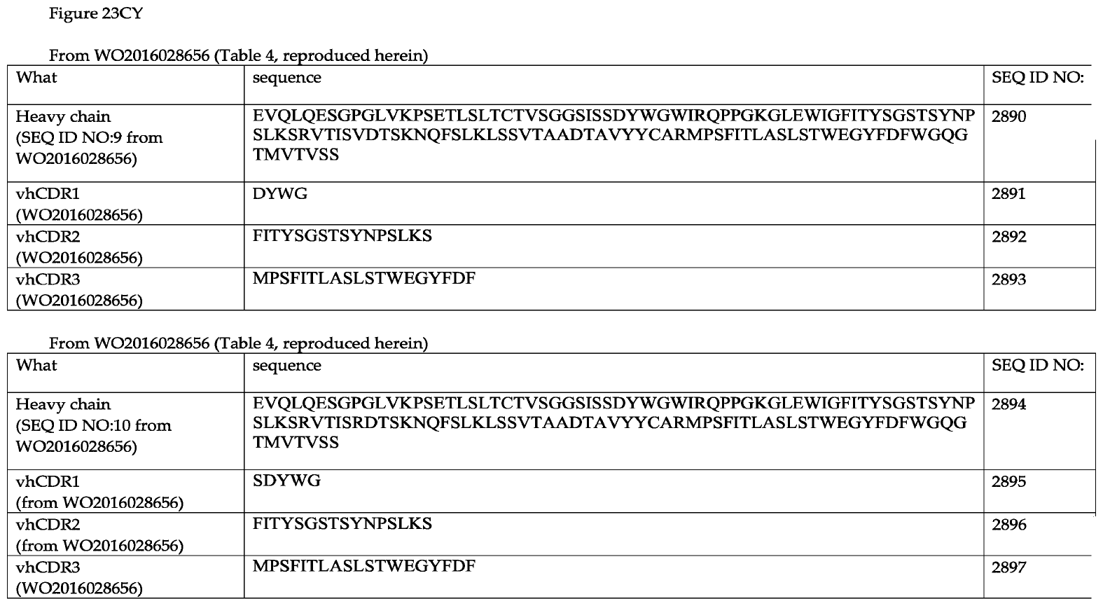

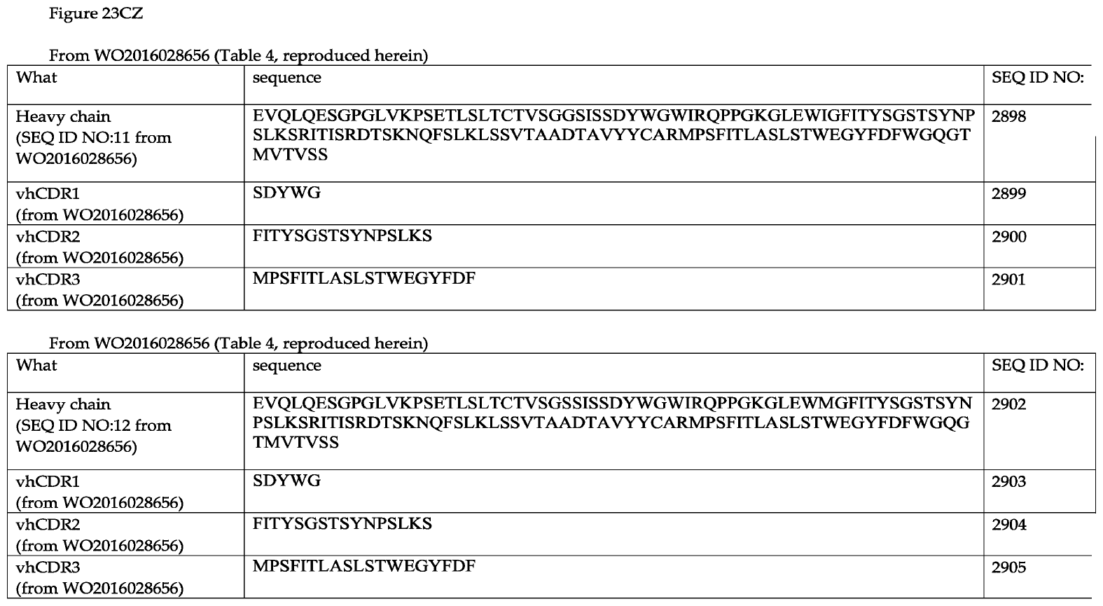

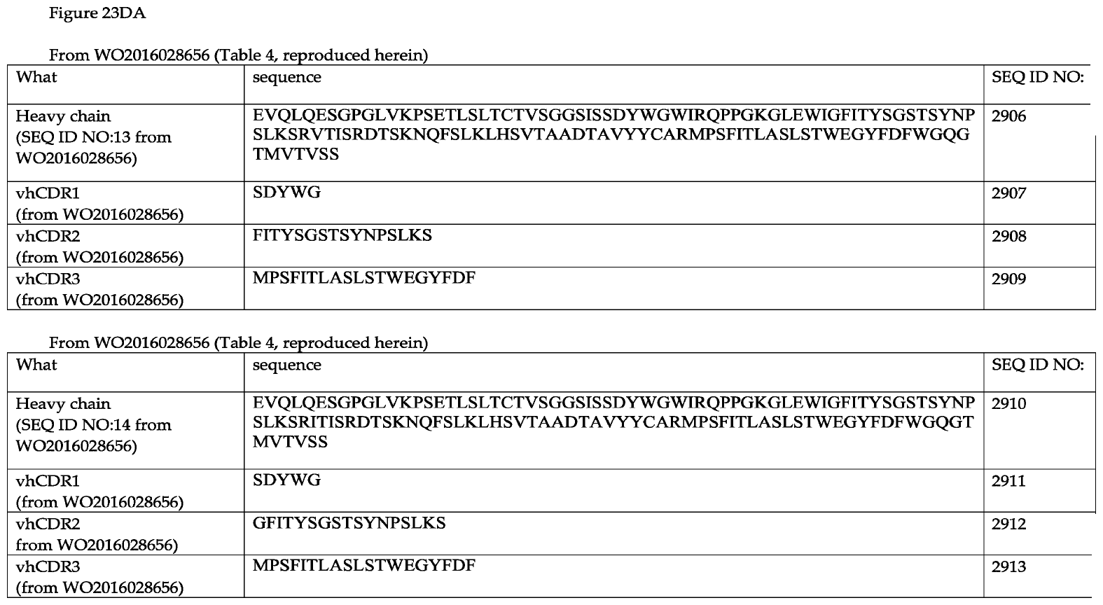

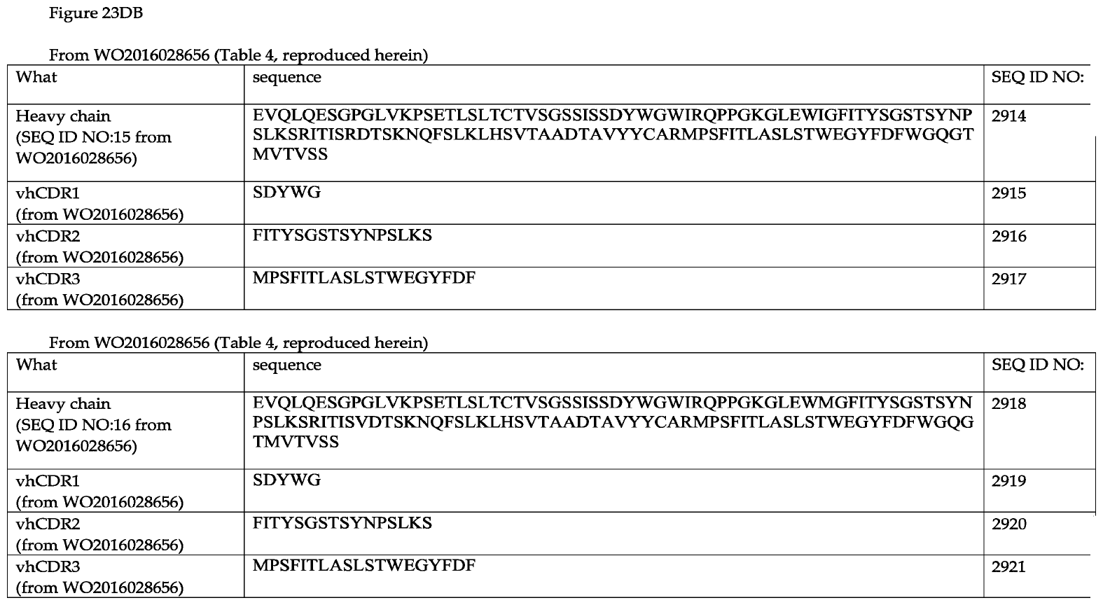

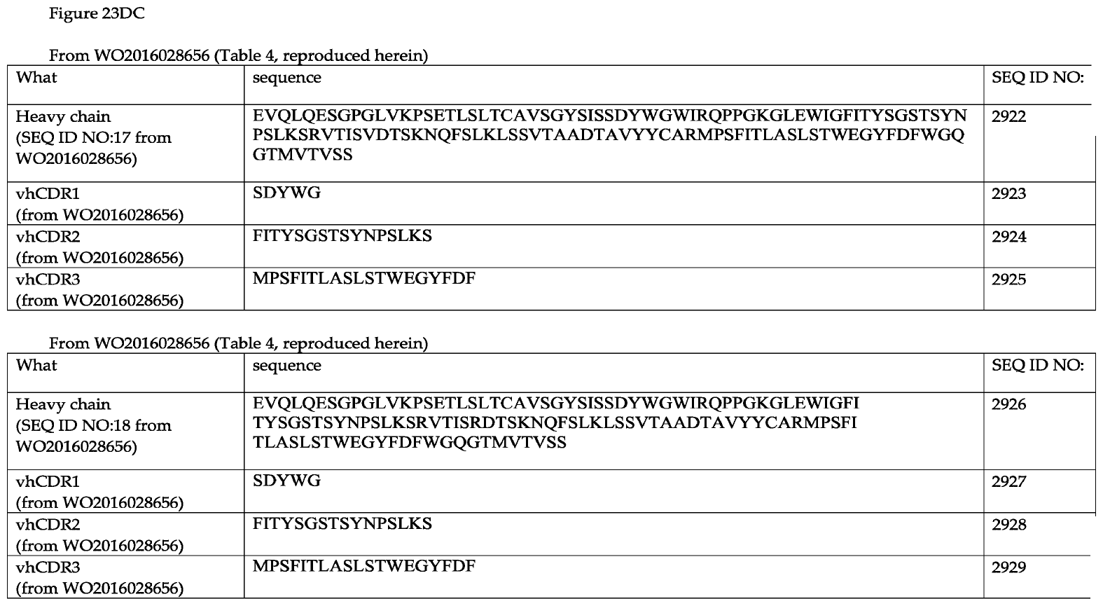

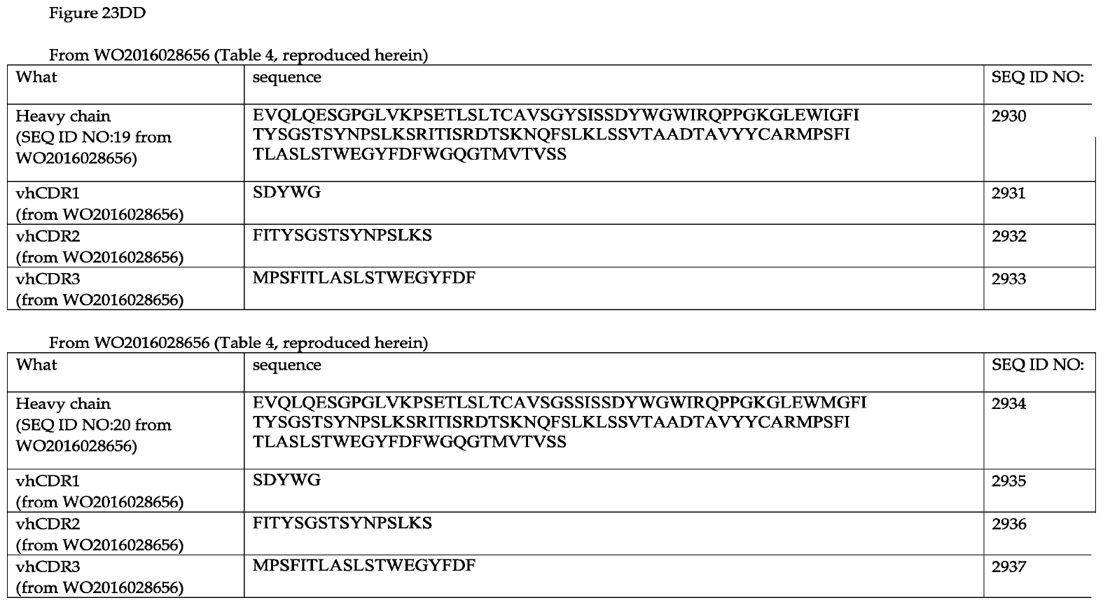

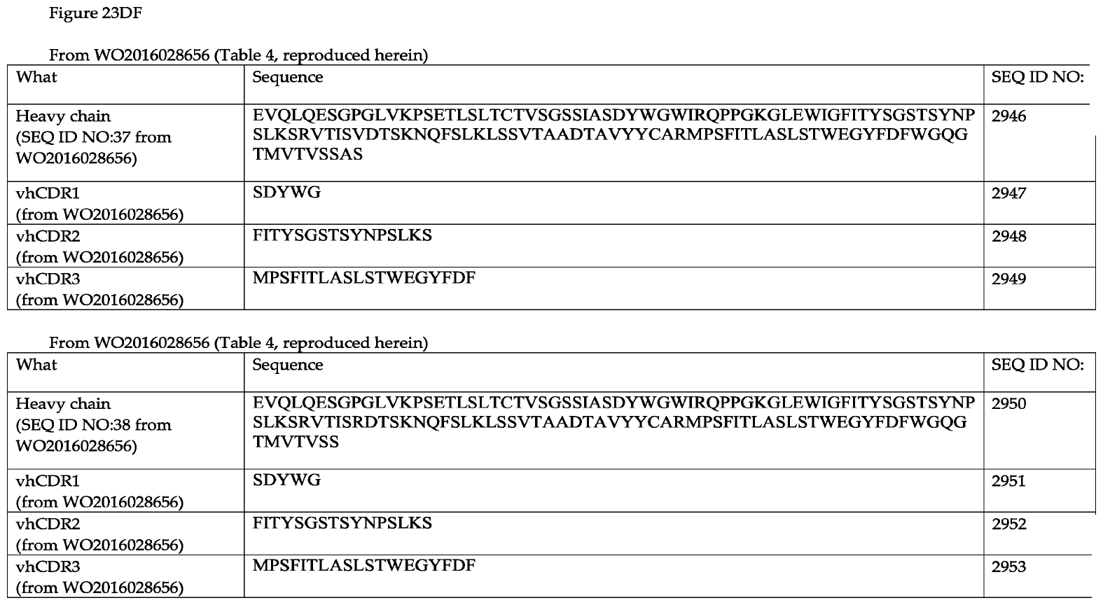

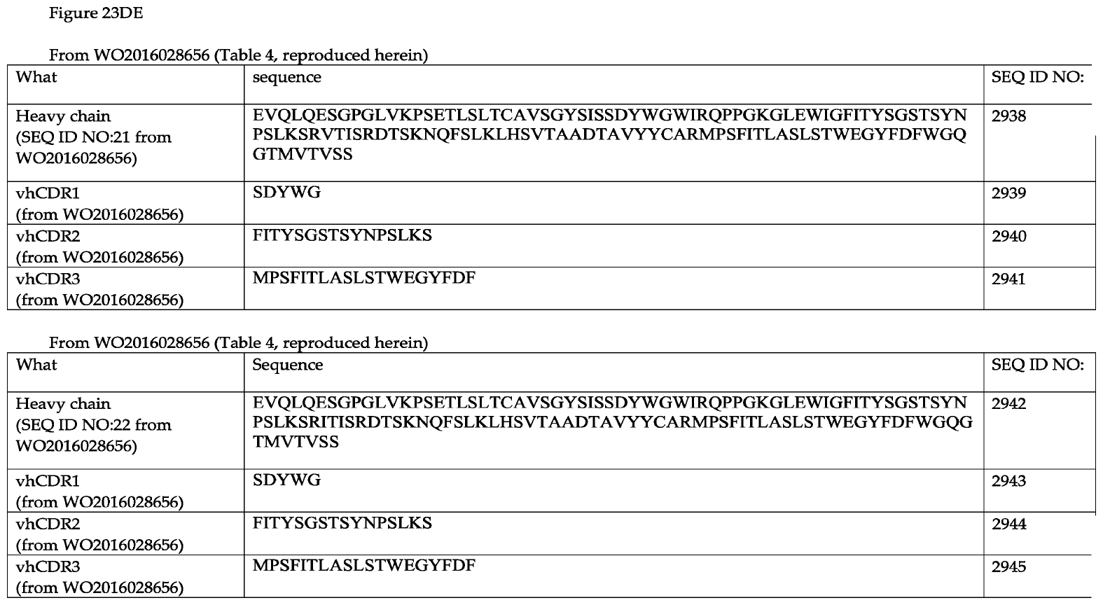

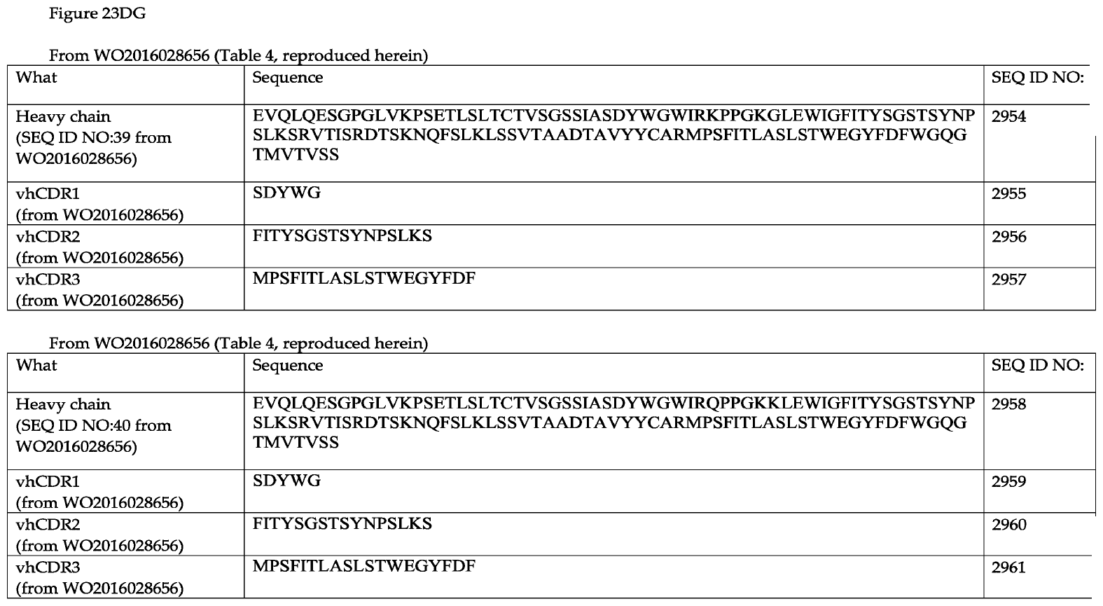

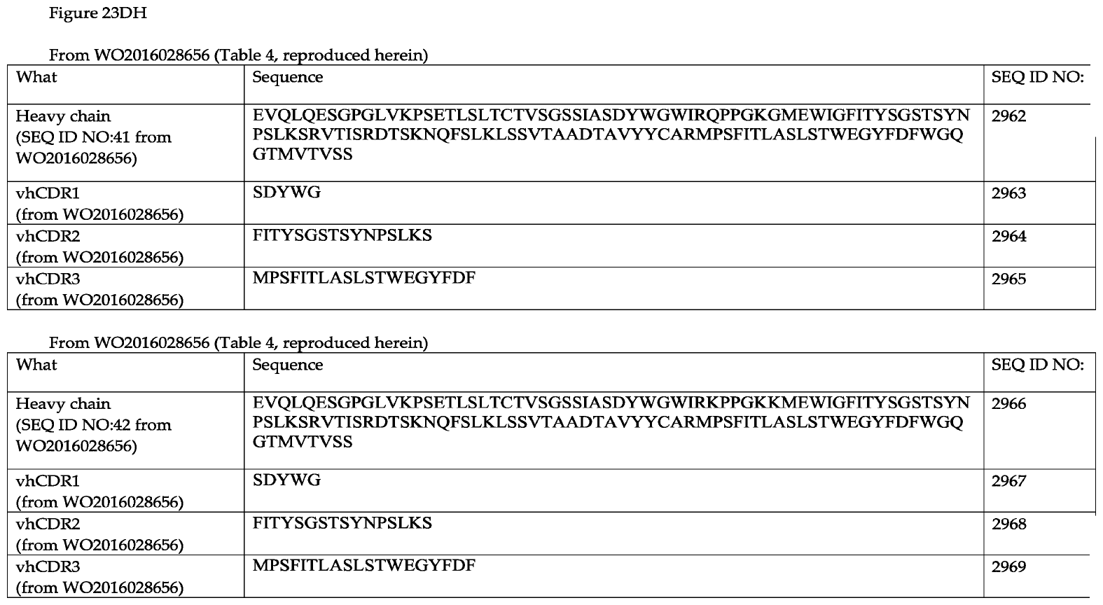

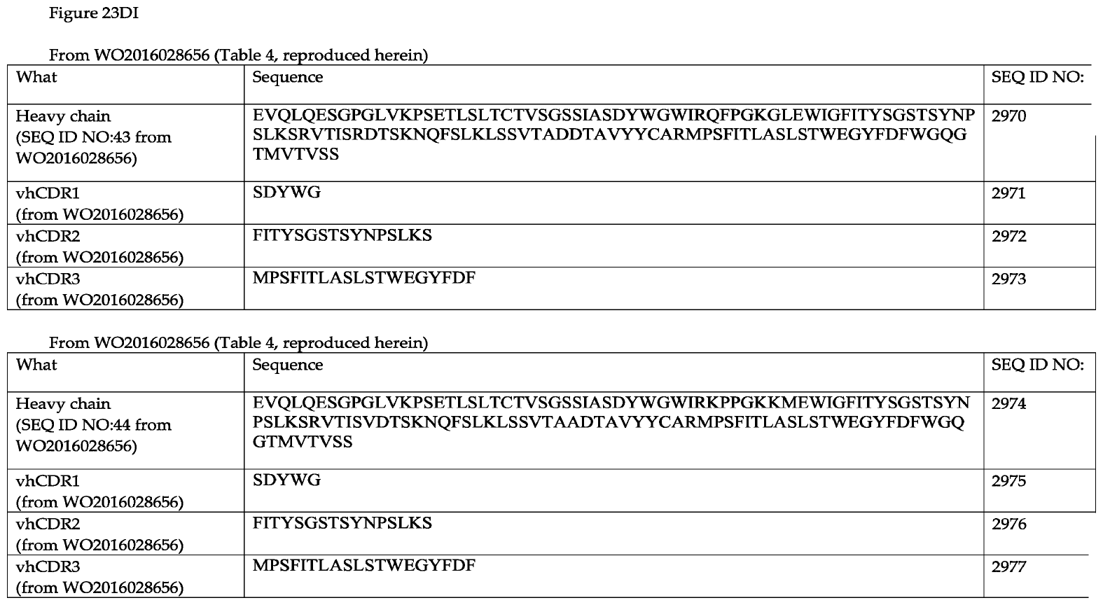

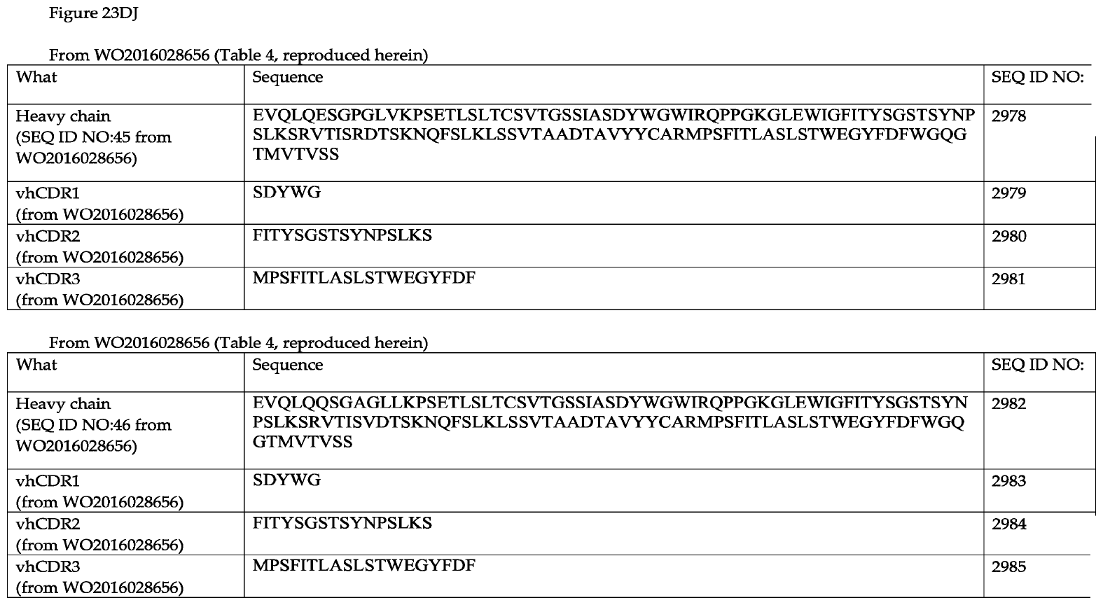

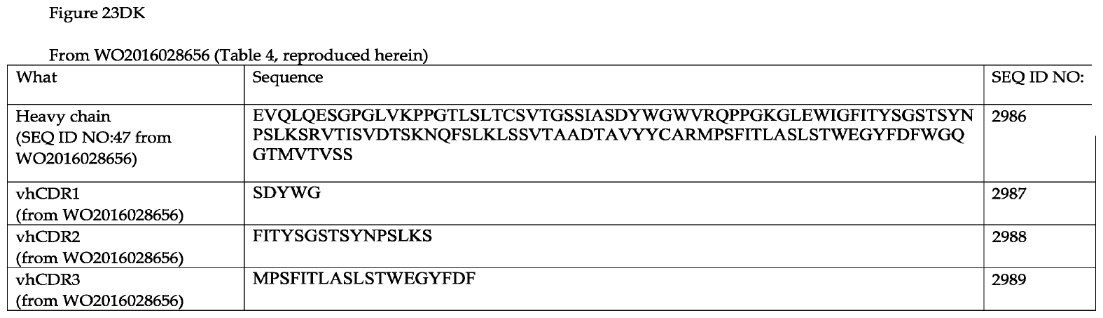

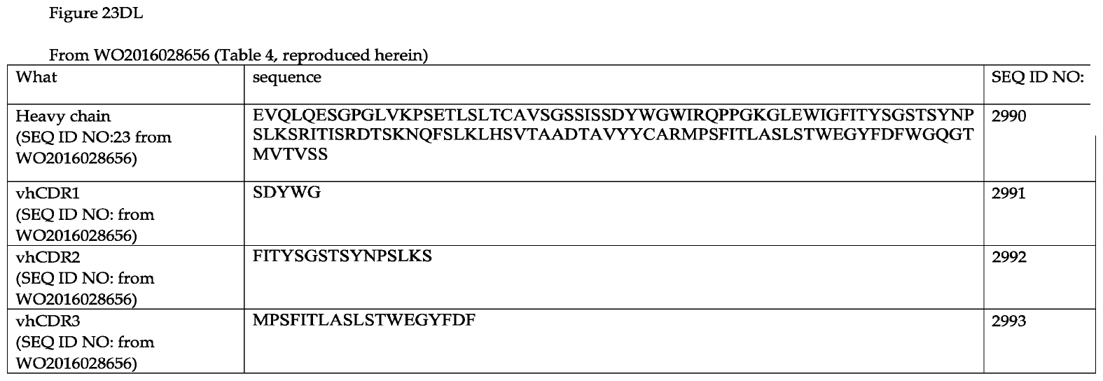

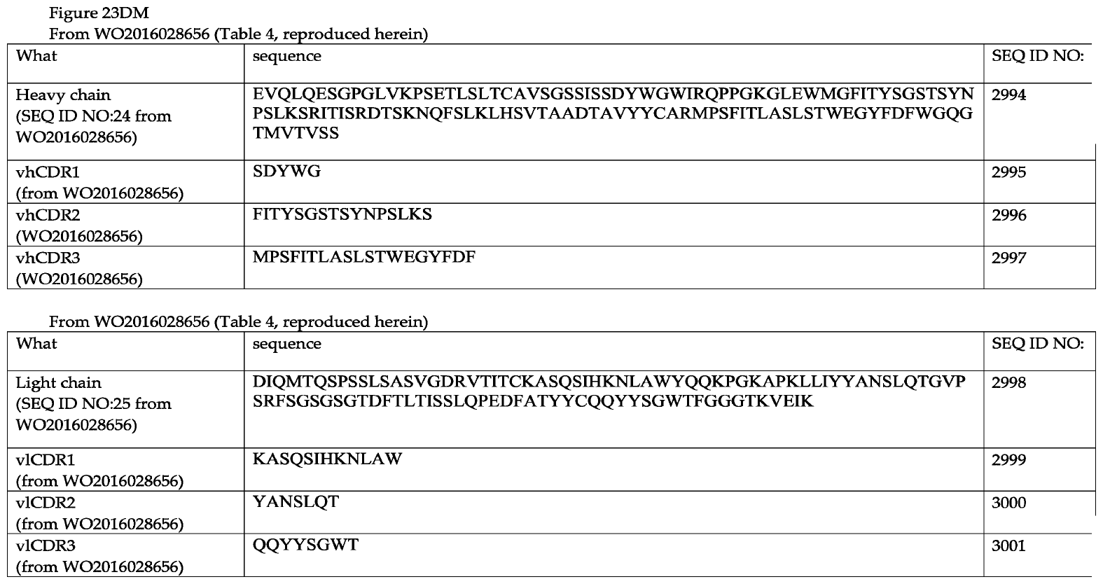

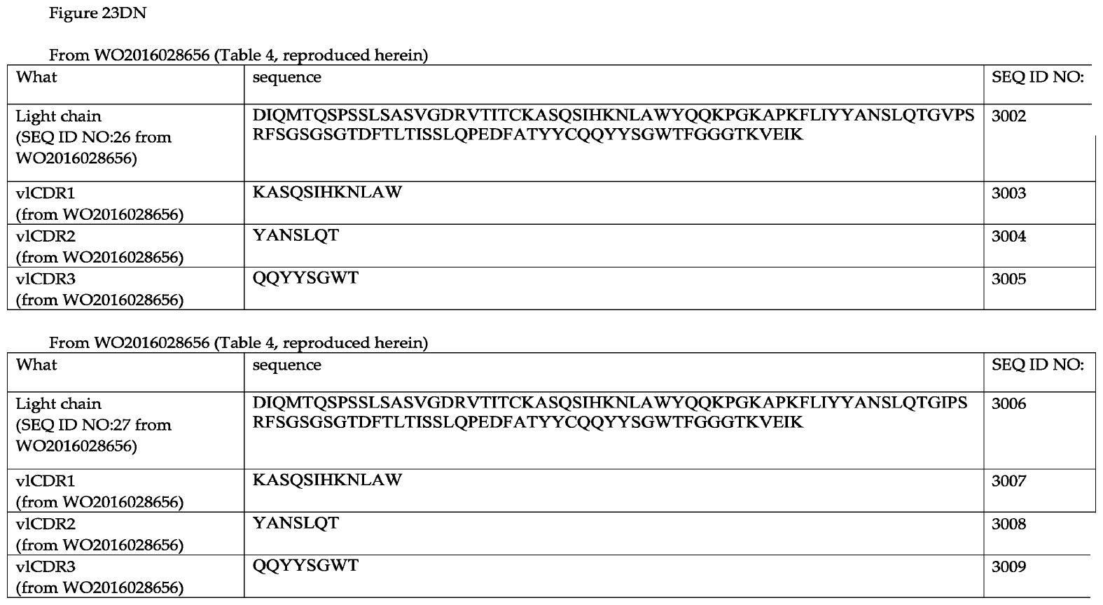

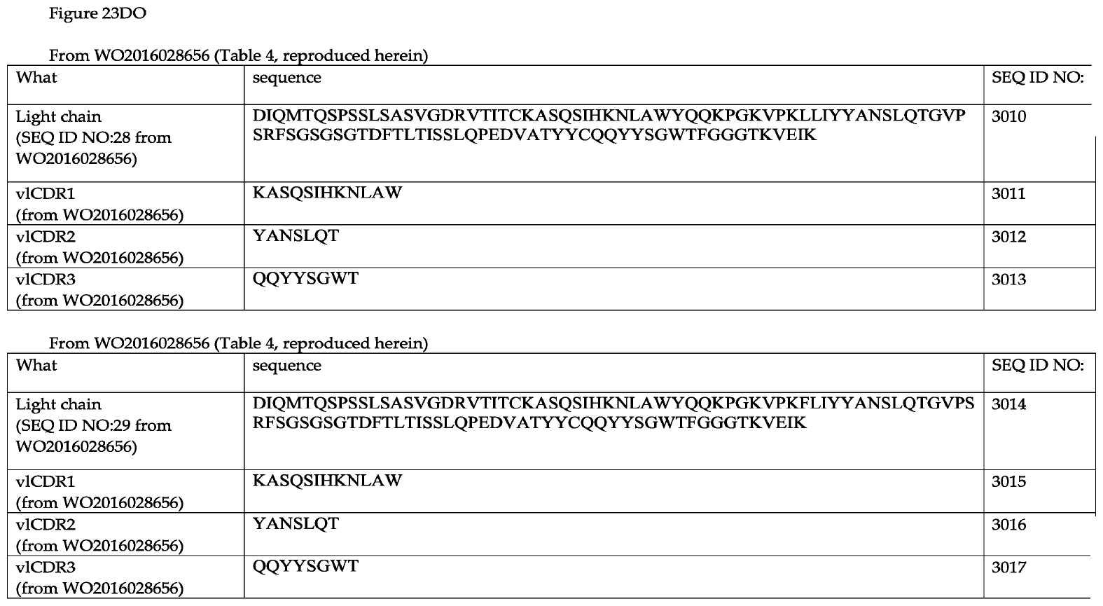

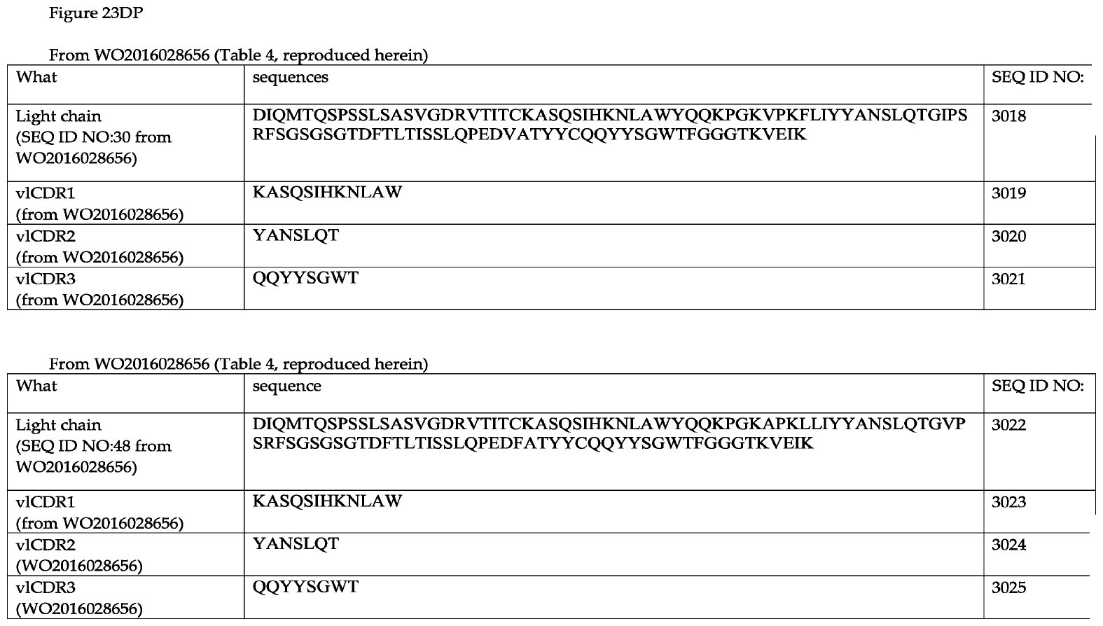

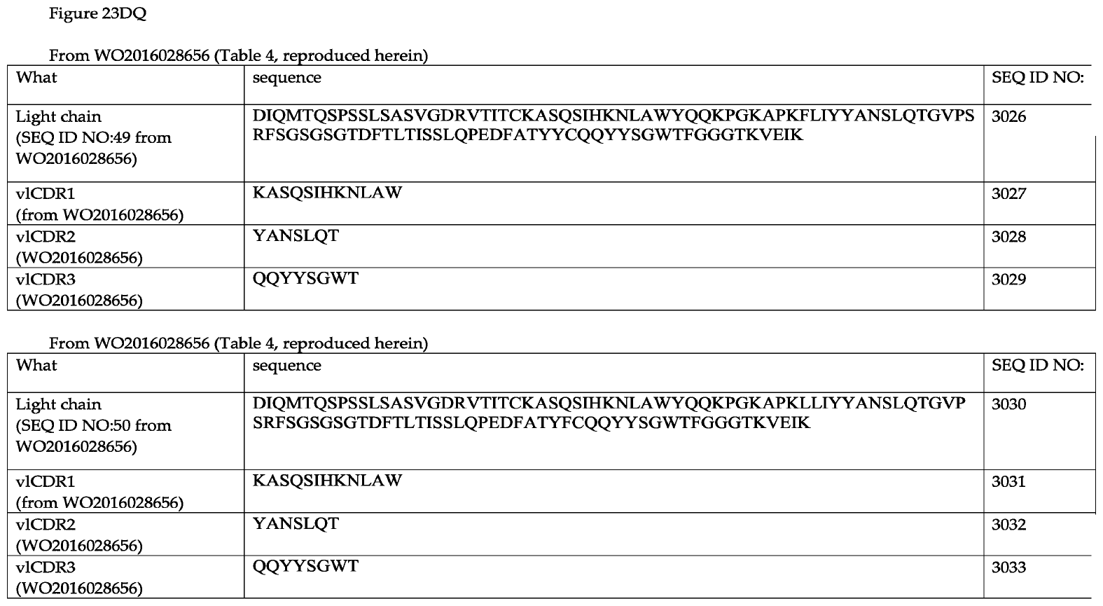

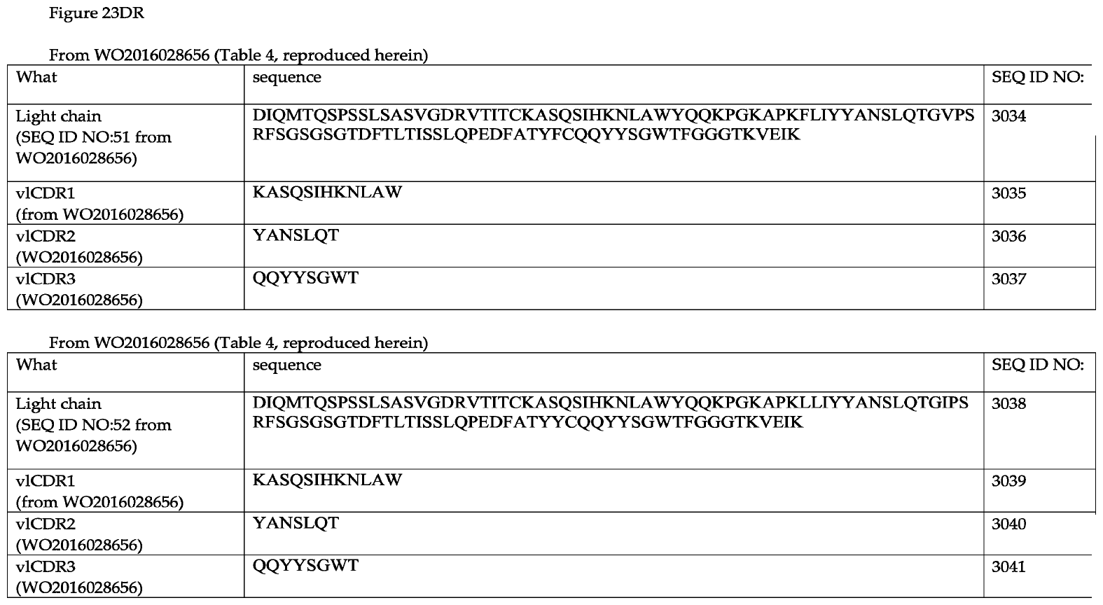

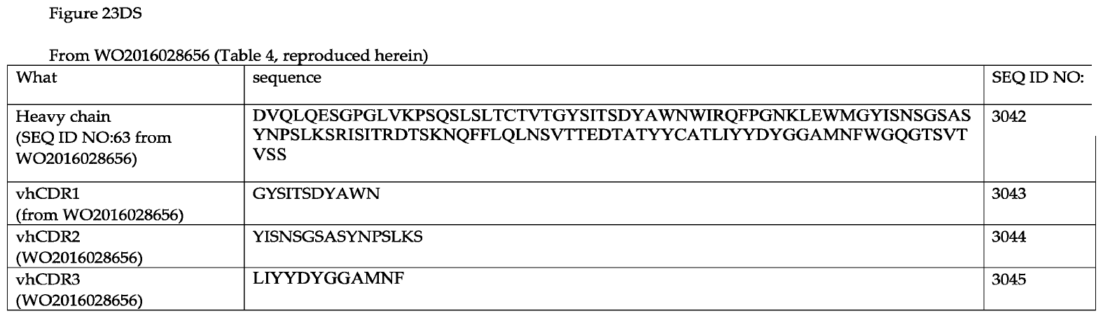

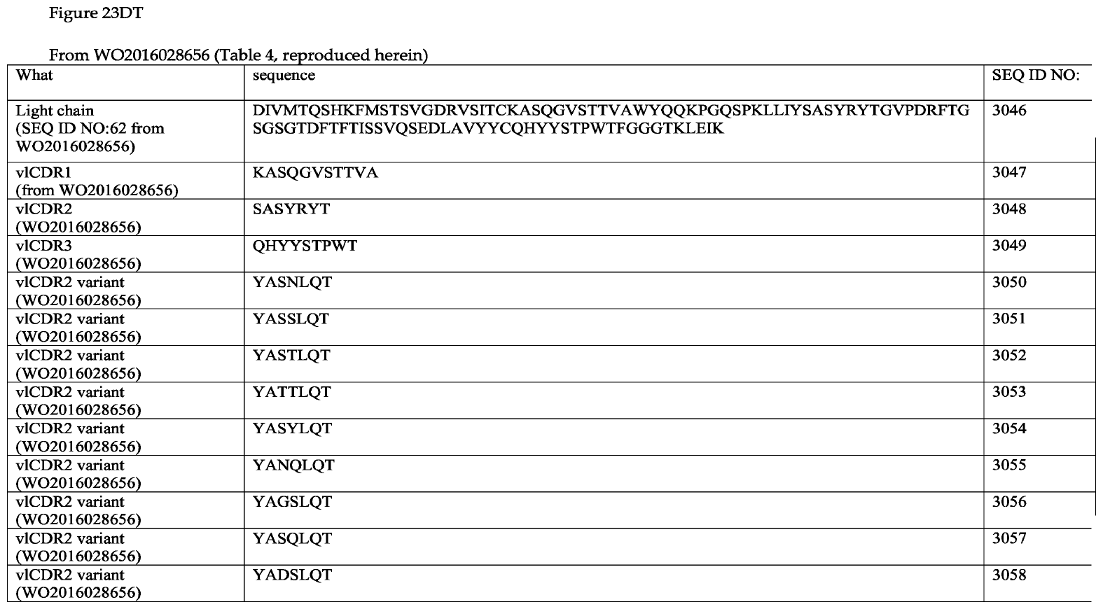

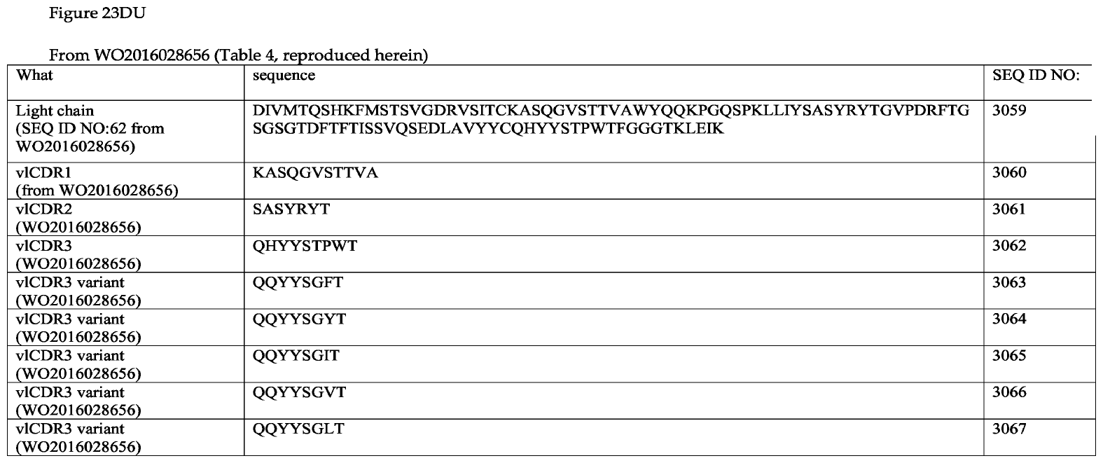

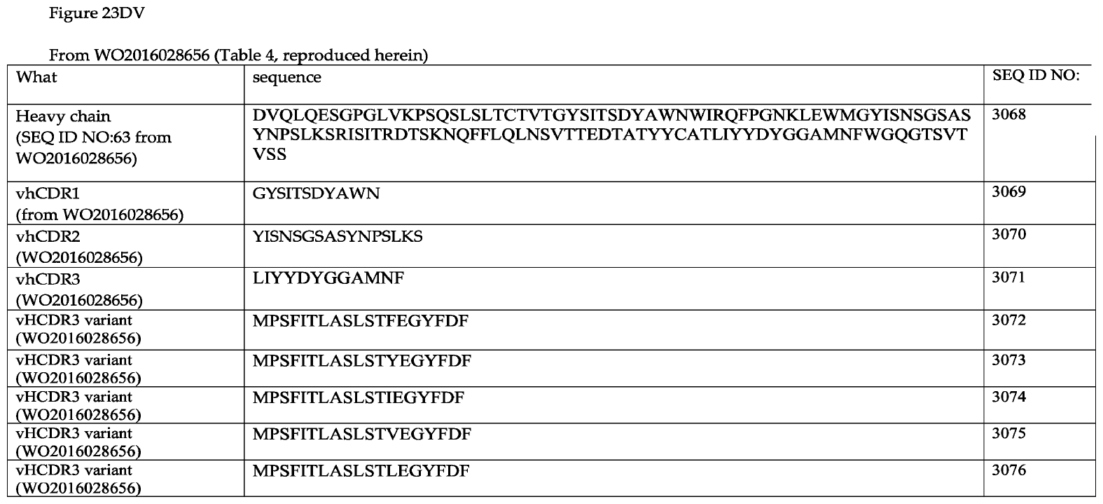

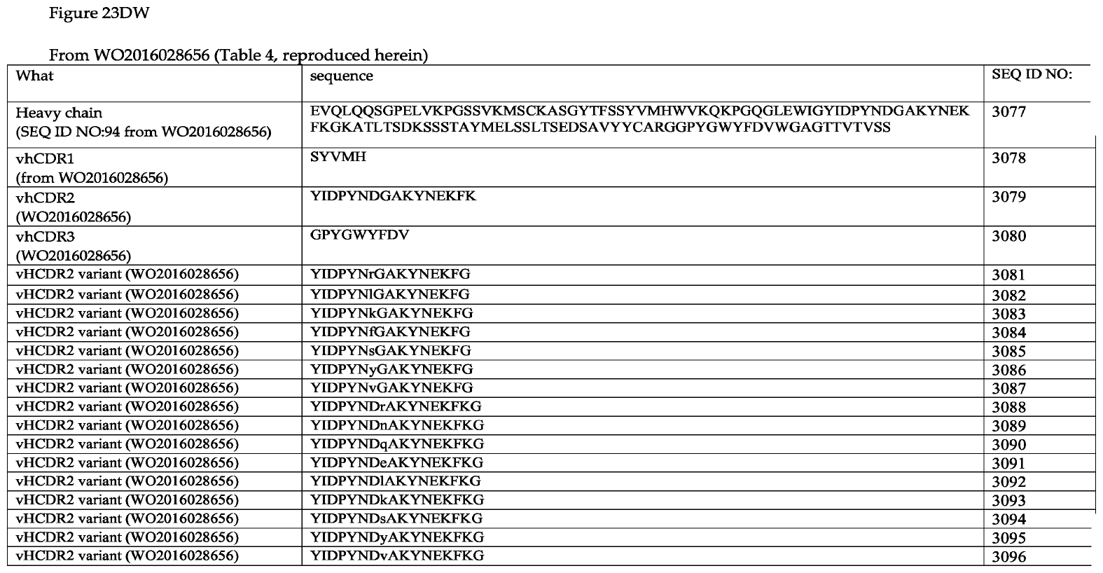

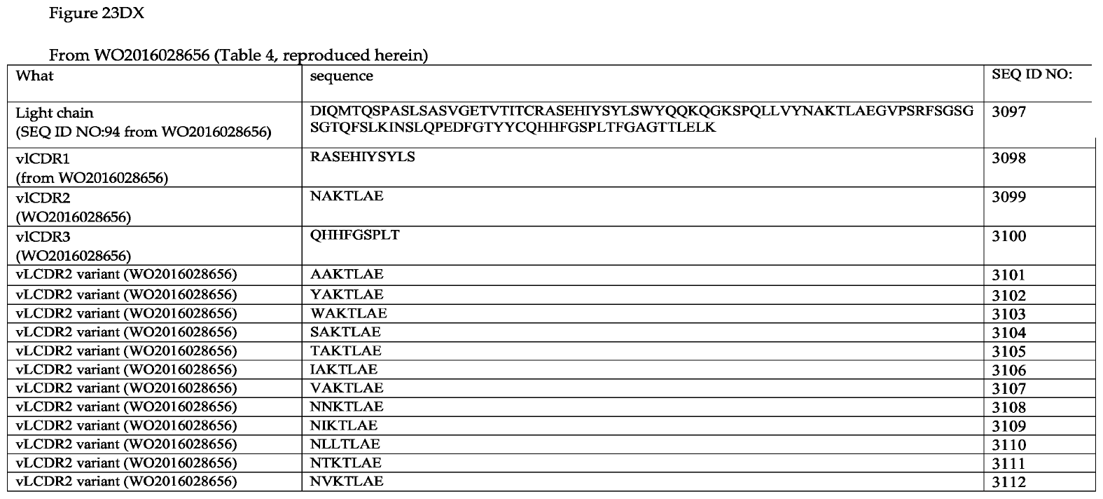

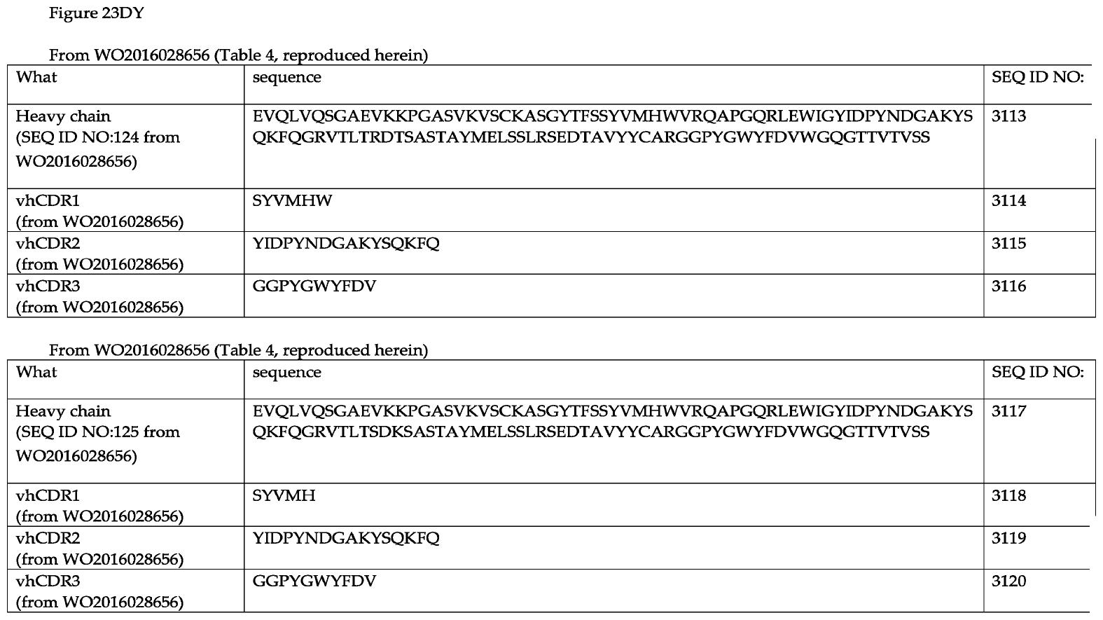

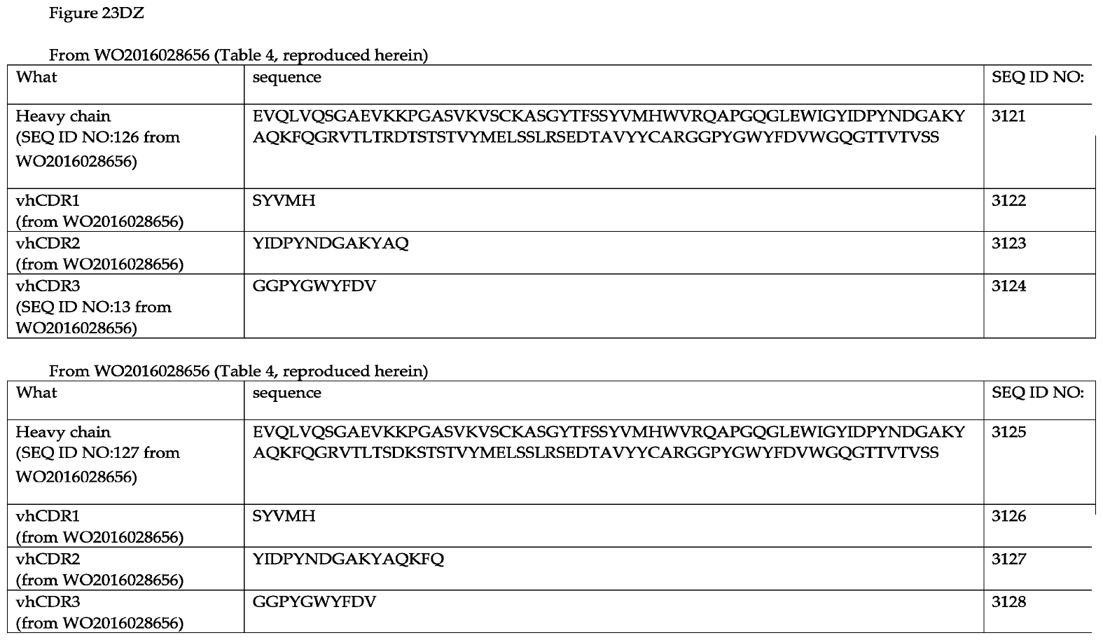

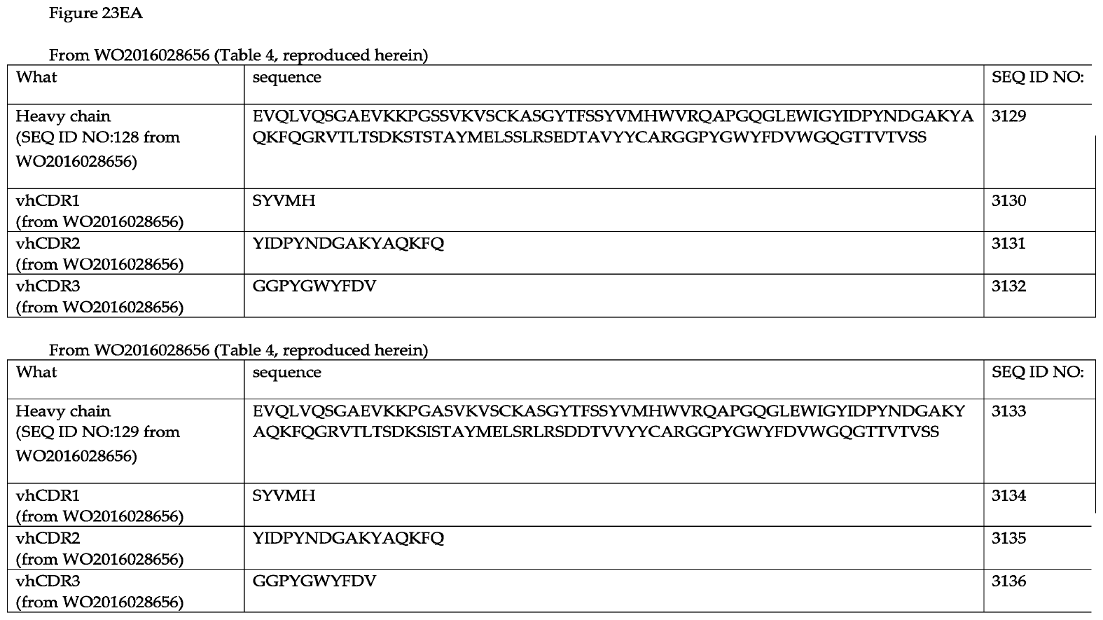

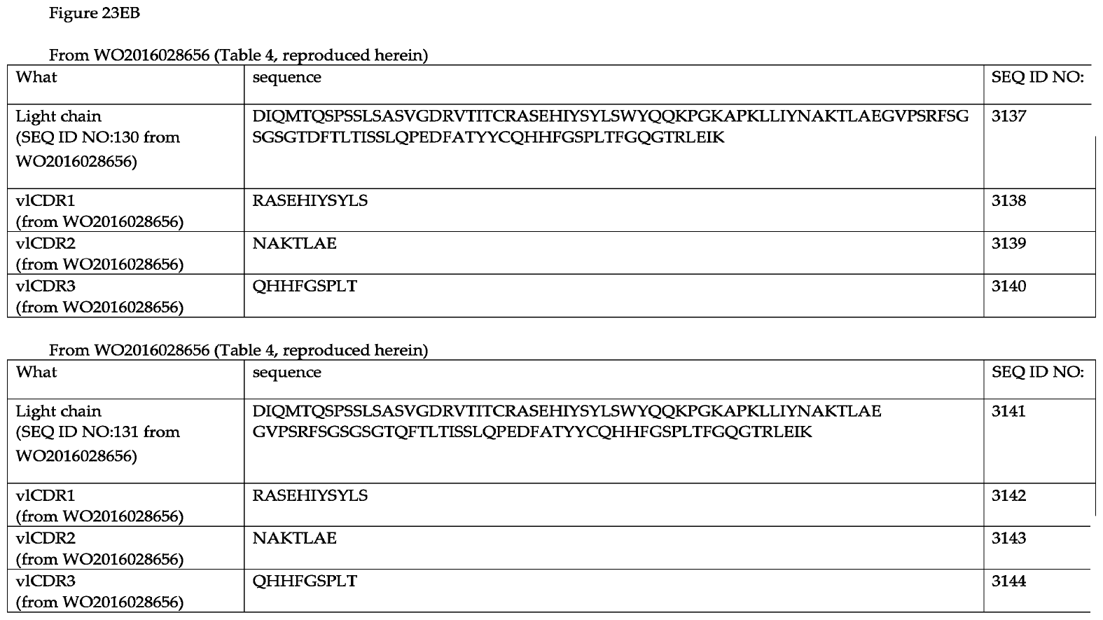

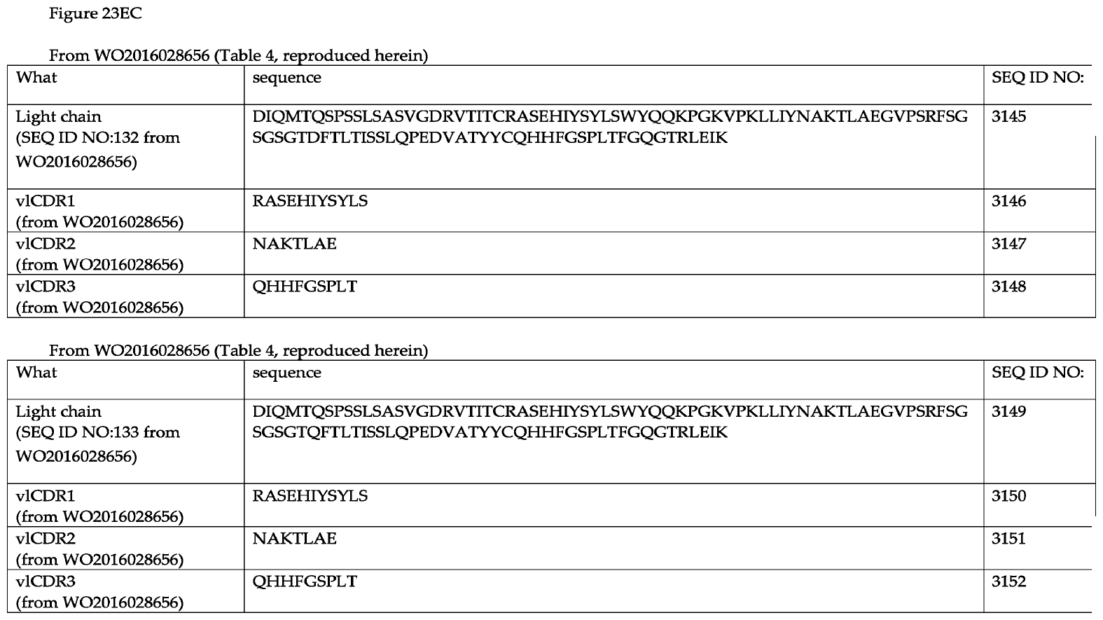

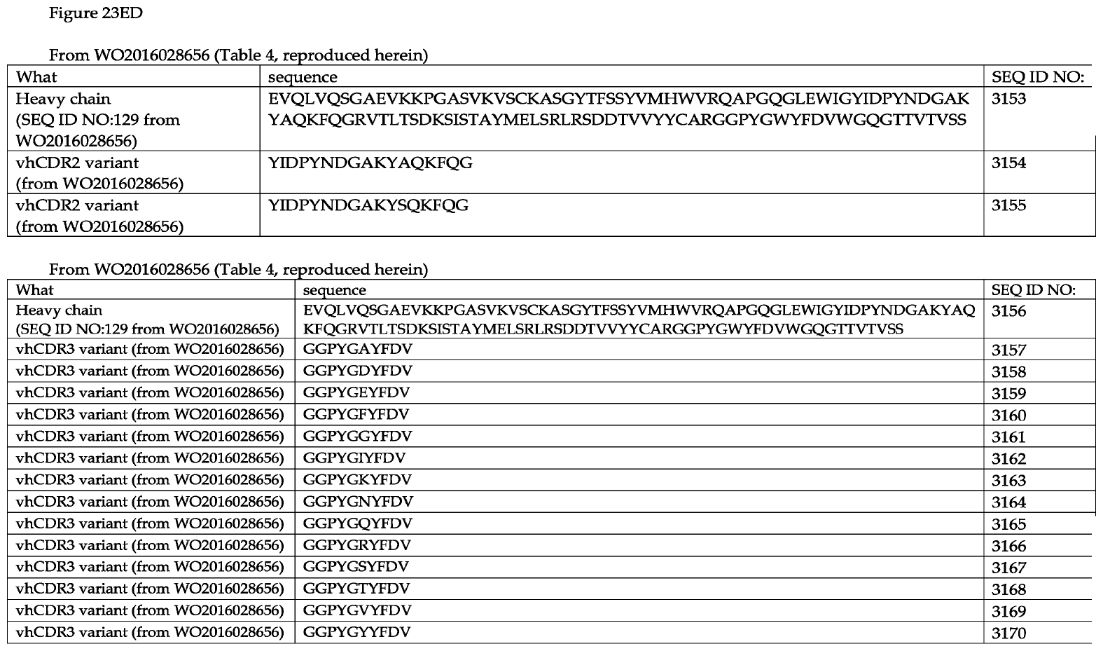

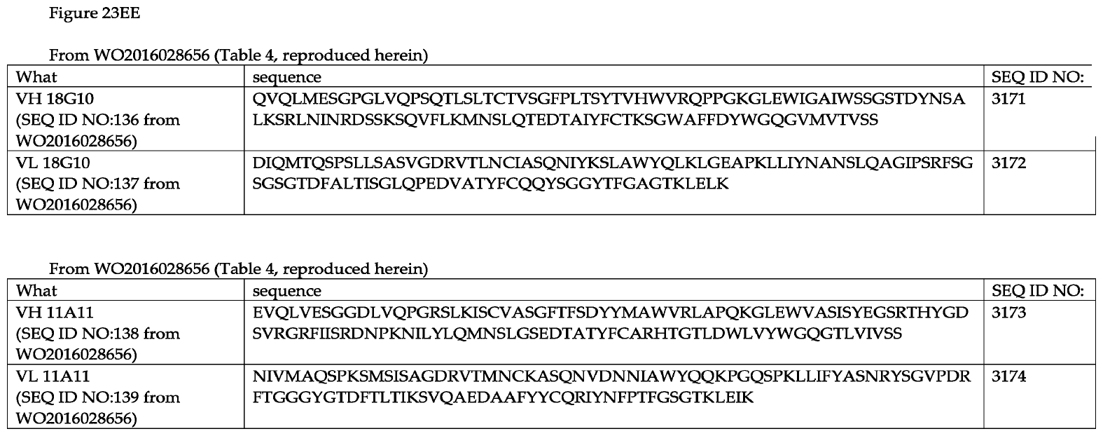

In some embodiments, the invention provides scFvs that bind to TIGIT comprising a variable heavy domain and a variable light domain linked by an scFv linker as outlined above. The VL and VH domains can be in either orientation, e.g. from N- to C-terminus “VH-linker-VL” or “VL-linker-VH”. These are named by their component parts; for example, “scFv-CPA. 9.086.VH-linker-VL” or “scFv-CPA.9.086.VL-linker-VH.” Thus, “scFv-CPA.9.086” can be in either orientation. The/anti-TIGIT antibodies for use with the invention can comprise any scFvs that bind to TIGIT as part of the TIGIT binding portion. The anti-TIGIT antibodies for use with the invention can comprise any scFvs that bind to TIGIT as part of the TIGIT antigen binding domain. In many embodiments, the antibodies for use with the invention are human (derived from phage) and block binding of TIGIT and PVR. Antibodies that both bind and block the receptor-ligand interaction are as below, with their components outlined as well (as discussed in the “Sequence” section, the sequences of all but the scFv constructs are in the sequence listing as well as provided in FIG. 23A-23EE):

CPA.9.018, CPA.9.018.VH, CPA.9.018.VL, CPA.9.018.HC, CPA.9.018.LC, CPA.9.018.H1, CPA.9.018.H2, CPA.9.018.H3, CPA.9.018.H4; CPA.9.018.H4(S241P); CPA.9.018.vhCDR1, CPA.9.018.vhCDR2, CPA.9.018.vhCDR3, CPA.9.018.vlCDR1, CPA.9.018.vlCDR2, CPA.9.018.vlCDR3 and scFv-CPA.9.018;

CPA.9.027, CPA.9.027.VH, CPA.9.027.VL, CPA.9.027.HC, CPA.9.027.LC, CPA.9.027.H1, CPA.9.027.H2, CPA.9.027.H3, CPA.9.027.H4; CPA.9.018.H4(S241P); CPA.9.027.vhCDR1, CPA.9.027.vhCDR2, CPA.9.027.vhCDR3, CPA.9.027.vlCDR1, CPA.9.027.vlCDR2, CPA.9.027.vlCDR3 and scFv-CPA.9.027;

CPA.9.049, CPA.9.049.VH, CPA.9.049.VL, CPA.9.049.HC, CPA.9.049.LC, CPA.9.049.H1, CPA.9.049.H2, CPA.9.049.H3; CPA.9.049.H4; CPA.9.049.H4(S241P); CPA.9.049.vhCDR1, CPA.9.049.vhCDR2, CPA.9.049.vhCDR3, CPA.9.049.vlCDR1, CPA.9.049.vlCDR2, CPA.9.049.vlCDR3 and scFv-CPA.9.049;

CPA.9.057, CPA.9.057.VH, CPA.9.057.VL, CPA.9.057.HC, CPA.9.057.LC, CPA.9.057.H1, CPA.9.057.H2, CPA.9.057.H3; CPA.9.057.H4; CPA.9.057.H4(S241P); CPA.9.057.vhCDR1, CPA.9.057.vhCDR2, CPA.9.057.vhCDR3, CPA.9.057.vlCDR1, CPA.9.057.vlCDR2, CPA.9.057.vlCDR3 and scFv-CPA.9.057;

CPA.9.059, CPA.9.059.VH, CPA.9.059.VL, CPA.9.059.HC, CPA.9.059.LC, CPA.9.059.H1, CPA.9.059.H2, CPA.9.059.H3; CPA.9.059.H4; CPA.9.059.H4(S241P); CPA.9.059.vhCDR1, CPA.9.059.vhCDR2, CPA.9.059.vhCDR3, CPA.9.059.vlCDR1, CPA.9.059.vlCDR2, CPA.9.059.vlCDR3 and scFv-CPA.9.059;

CPA.9.083, CPA.9.083.VH, CPA.9.083.VL, CPA.9.083.HC, CPA.9.083.LC, CPA.9.083.H1, CPA.9.083.H2, CPA.9.083.H3; CPA.9.083.H4; CPA.9.083.H4(S241P); CPA.9.083.vhCDR1, CPA.9.083.vhCDR2, CPA.9.083.vhCDR3, CPA.9.083.vlCDR1, CPA.9.083.vlCDR2, CPA.9.083.vlCDR3 and scFv-CPA.9.083;

CPA.9.086, CPA.9.086.VH, CPA.9.086.VL, CPA.9.086.HC, CPA.9.086.LC, CPA.9.086.H1, CPA.9.086.H2, CPA.9.086.H3; CPA.9.086.H4; CPA.9.086.H4(S241P); CPA.9.086.vhCDR1, CPA.9.086.vhCDR2, CPA.9.086.vhCDR3, CPA.9.086.vlCDR1, CPA.9.086.vlCDR2, CPA.9.086.vlCDR3 and scFv-CPA.9.086;

CPA.9.089, CPA.9.089.VH, CPA.9.089.VL, CPA.9.089.HC, CPA.9.089.LC, CPA.9.089.H1, CPA.9.089.H2, CPA.9.089.H3; CPA.9.089.H4; CPA.9.089.H4(S241P); CPA.9.089.vhCDR1, CPA.9.089.vhCDR2, CPA.9.089.vhCDR3, CPA.9.089.vlCDR1, CPA.9.089.vlCDR2, CPA.9.089.vlCDR3 and scFv-CPA.9.089;

CPA.9.093, CPA.9.093.VH, CPA.9.093.VL, CPA.9.093.HC, CPA.9.093.LC, CPA.9.093.H1, CPA.9.093.H2, CPA.9.093.H3; CPA.9.093.H4; CPA.9.093.H4(S241P); CPA.9.093.vhCDR1, CPA.9.093.vhCDR2, CPA.9.093.vhCDR3, CPA.9.093.vlCDR1, CPA.9.093.vlCDR2, CPA.9.093.vlCDR3 and scFv-CPA.9.093;

CPA.9.101, CPA.9.101.VH, CPA.9.101.VL, CPA.9.101.HC, CPA.9.101.LC, CPA.9.101.H1, CPA.9.101.H2, CPA.9.101.H3; CPA.9.101.H4; CPA.9.101.H4(S241P); CPA.9.101.vhCDR1, CPA.9.101.vhCDR2, CPA.9.101.vhCDR3, CPA.9.101.vlCDR1, CPA.9.101.vlCDR2, CPA.9.101.vlCDR3 and scFv-CPA.9.101; and

CPA.9.103, CPA.9.103.VH, CPA.9.103.VL, CPA.9.103.HC, CPA.9.103.LC, CPA.9.103.H1, CPA.9.103.H2, CPA.9.103.H3; CPA.9.103.H4; CPA.9.103.H4(S241P); CPA.9.103.vhCDR1, CPA.9.103.vhCDR2, CPA.9.103.vhCDR3, CPA.9.103.vlCDR1, CPA.9.103.vlCDR2, CPA.9.103.vlCDR3 and scFv-CPA.9.103.

Furthermore, the present invention provides a number of CHA antibodies, which are murine antibodies generated from hybridomas. As is well known the art, the six CDRs are useful when put into either human framework variable heavy and variable light regions or when the variable heavy and light domains are humanized. Accordingly, the present invention provides antibodies, usually full length or scFv domains, that comprise the following sets of CDRs, the sequences of which are shown in FIG. 22A-22D and/or the sequence listing:

CHA.9.536.1, CHA.9.536.1.VH, CHA.9.536.1.VL, CHA.9.536.1.HC, CHA.9.536.1.LC, CHA.9.536.1.H1, CHA.9.536.1.H2, CHA.9.536.1.H3; CHA.9.536.1.H4, CHA.9.536.1.H4(S241P), CHA.9.536.1.vhCDR1, CHA.9.536.1.vhCDR2, CHA.9.536.1.vhCDR3, CHA.9.536.1.vlCDR1, CHA.9.536.1.vlCDR2 and CHA.9.536.1.vhCDR3;

CHA.9.536.3, CHA.9.536.3.VH, CHA.9.536.3.VL, CHA.9.536.3.HC, CHA.9.536.3.LC, CHA.9.536.3.H1, CHA.9.536.3.H2, CHA.9.536.3.H3; CHA.9.536.3.H4, CHA.9.536.3.H4(S241P); CHA.9.536.3.vhCDR1, CHA.9.536.3.vhCDR2, CHA.9.536.3.vhCDR3, CHA.9.536.3.vlCDR1, CHA.9.536.3.vlCDR2 and CHA.9.536.3.vhCDR3;

CHA.9.536.4, CHA.9.536.4.VH, CHA.9.536.4.VL, CHA.9.536.4.HC, CHA.9.536.4.LC, CHA.9.536.4.H1, CHA.9.536.4.H2, CHA.9.536.4.H3; CHA.9.536.4.H4, CHA.9.536.4.H4(S241P), CHA.9.536.4.vhCDR1, CHA.9.536.4.vhCDR2, CHA.9.536.4.vhCDR3, CHA.9.536.4.vlCDR1, CHA.9.536.4.vlCDR2 and CHA.9.536.4.vhCDR3;

CHA.9.536.5, CHA.9.536.5.VH, CHA.9.536.5.VL, CHA.9.536.5.HC, CHA.9.536.5.LC, CHA.9.536.5.H1, CHA.9.536.5.H2, CHA.9.536.5.H3; CHA.9.536.5.H4, CHA.9.536.5.H4(S241P), CHA.9.536.5.vhCDR1, CHA.9.536.5.vhCDR2, CHA.9.536.5.vhCDR3, CHA.9.536.5.vlCDR1, CHA.9.536.5.vlCDR2 and CHA.9.536.5.vhCDR3;

CHA.9.536.6, CHA.9.536.6.VH, CHA.9.536.6.VL, CHA.9.536.6.HC, CHA.9.536.6.LC, CHA.9.536.6.H1, CHA.9.536.6.H2, CHA.9.536.6.H3; CHA.9.536.6.H4, CHA.9.536.6.vhCDR1, CHA.9.536.6.vhCDR2, CHA.9.536.6.vhCDR3, CHA.9.536.6.vlCDR1, CHA.9.536.6.vlCDR2 and CHA.9.536.6.vhCDR3;

CHA.9.536.7, CHA.9.536.7.VH, CHA.9.536.7.VL, CHA.9.536.7.HC, CHA.9.536.7.LC, CHA.9.536.7.H1, CHA.9.536.7.H2, CHA.9.536.7.H3; CHA.9.536.7.H4, CHA.9.536.5.H4(S241P); CHA.9.536.7.vhCDR1, CHA.9.536.7.vhCDR2, CHA.9.536.7.vhCDR3, CHA.9.536.7.vlCDR1, CHA.9.536.7.vlCDR2 and CHA.9.536.7.vhCDR3;

CHA.9.536.8, CHA.9.536.8.VH, CHA.9.536.8.VL, CHA.9.536.8.HC, CHA.9.536.8.LC, CHA.9.536.8.H1, CHA.9.536.8.H2, CHA.9.536.8.H3; CHA.9.536.8.H4, CHA.9.536.8.H4(S241P), CHA.9.536.8.vhCDR1, CHA.9.536.8.vhCDR2, CHA.9.536.8.vhCDR3, CHA.9.536.8.vlCDR1, CHA.9.536.8.vlCDR2 and CHA.9.536.8.vhCDR3;

CHA.9.560.1, CHA.9.560.1VH, CHA.9.560.1.VL, CHA.9.560.1.HC, CHA.9.560.1.LC, CHA.9.560.1.H1, CHA.9.560.1.H2, CHA.9.560.1.H3; CHA.9.560.1.H4, CHA.9.560.1.H4(S241P), CHA.9.560.1.vhCDR1, CHA.9.560.1.vhCDR2, CHA.9.560.1.vhCDR3, CHA.9.560.1.vlCDR1, CHA.9.560.1.vlCDR2 and CHA.9.560.1.vhCDR3;

CHA.9.560.3, CHA.9.560.3VH, CHA.9.560.3.VL, CHA.9.560.3.HC, CHA.9.560.3.LC, CHA.9.560.3.H1, CHA.9.560.3.H2, CHA.9.560.3.H3; CHA.9.560.3.H4, CHA.9.560.3.H4(S241P); CHA.9.560.3.vhCDR1, CHA.9.560.3.vhCDR2, CHA.9.560.3.vhCDR3, CHA.9.560.3.vlCDR1, CHA.9.560.3.vlCDR2 and CHA.9.560.3.vhCDR3;

CHA.9.560.4, CHA.9.560.4VH, CHA.9.560.4.VL, CHA.9.560.4.HC, CHA.9.560.4.LC, CHA.9.560.4.H1, CHA.9.560.4.H2, CHA.9.560.4.H3; CHA.9.560.4.H4, CHA.9.560.4.H4(S241P), CHA.9.560.4.vhCDR1, CHA.9.560.4.vhCDR2, CHA.9.560.4.vhCDR3, CHA.9.560.4.vlCDR1, CHA.9.560.4.vlCDR2 and CHA.9.560.4.vhCDR3;

CHA.9.560.5, CHA.9.560.5VH, CHA.9.560.5.VL,CHA.9.560.5.HC, CHA.9.560.5.LC, CHA.9.560.5.H1, CHA.9.560.5.H2, CHA.9.560.5.H3; CHA.9.560.5.H4, CHA.9.560.5.vhCDR1, CHA.9.560.5.vhCDR2, CHA.9.560.5.vhCDR3, CHA.9.560.5.vlCDR1, CHA.9.560.5.vlCDR2 and CHA.9.560.5.vhCDR3;

CHA.9.560.6, CHA.9.560.6VH, CHA.9.560.6.VL, CHA.9.560.6.HC, CHA.9.560.6.LC, CHA.9.560.6.H1, CHA.9560.6.H2, CHA.9.560.6.H3; CHA.9.560.6.H4, CHA.9.560.6.H4(S241P), CHA.9.560.6.vhCDR1, CHA.9.560.6.vhCDR2, CHA.9.560.6.vhCDR3, CHA.9.560.6.vlCDR1, CHA.9.560.6.vlCDR2 and CHA.9.560.6.vhCDR3;

CHA.9.560.7, CHA.9.560.7VH, CHA.9.560.7.VL, CHA.9.560.7.HC, CHA.9.560.7.LC, CHA.9.560.7.H1, CHA.9.560.7.H2, CHA.9.560.7.H3; CHA.9.560.7.H4; CHA.9.560.7.H4(S241P); CHA.9.560.7.vhCDR1, CHA.9.560.7.vhCDR2, CHA.9.560.7.vhCDR3, CHA.9.560.7.vlCDR1, CHA.9.560.7.vlCDR2 and CHA.9.560.7.vhCDR3;

CHA.9.560.8, CHA.9.560.8VH, CHA.9.560.8.VL, CHA.9.560.8.HC, CHA.9.560.8.LC, CHA.9.560.8.H1, CHA.9.560.8.H2, CHA.9.560.8.H3; CHA.9.560.8.H4, CHA.9.560.8.H4(S241P); CHA.9.560.8.vhCDR1, CHA.9.560.8.vhCDR2, CHA.9.560.8.vhCDR3, CHA.9.560.8.vlCDR1, CHA.9.560.8.vlCDR2 and CHA.9.560.8.vhCDR3;

CHA.9.546.1, CHA.9.546.1VH, CHA.9.546.1.VL, CHA.9.546.1.HC, CHA.9.546.1.LC, CHA.9.546.1.H1, CHA.9.546.1.H2, CHA.9.546.1.H3; CHA.9.546.1.H4, CHA.9.546.1.H4(S241P), CHA.9.546.1.vhCDR1, CHA.9.546.1.vhCDR2, CHA.9.546.1.vhCDR3, CHA.9.546.1.vlCDR1, CHA.9.546.1.vlCDR2 and CHA.9.546.1.vhCDR3;

CHA.9.547.1, CHA.9.547.1VH, CHA.9.547.1.VL, CHA.9.547.1.HC, CHA.9.547.1.LC, CHA.9.547.1.H1, CHA.9.547.1.H2, CHA.9.547.1.H3; CHA.9.547.1.H4, CHA.9.547.1.H4(S241P), CHA.9.547.1.vhCDR1, CHA.9.547.1.vhCDR2, CHA.9.547.1.vhCDR3, CHA.9.547.1.vlCDR1, CHA.9.547.1.vlCDR2 and CHA.9.547.1.vhCDR3;

CHA.9.547.2, CHA.9.547.2VH, CHA.9.547.2.VL, CHA.9.547.2.HC, CHA.9.547.2.LC, CHA.9.547.2.H1, CHA.9.547.2.H2, CHA.9.547.2.H3; CHA.9.547.2.H4, CHA.9.547.2.H4(S241P), CHA.9.547.2.vhCDR1, CHA.9.547.2.vhCDR2, CHA.9.547. 2.vhCDR3, CHA.9.547.2.vlCDR1, CHA.9.547.2.vlCDR2 and CHA.9.547.2.vhCDR3;

CHA.9.547.3, CHA.9.547.3VH, CHA.9.547.3.VL, CHA.9.547.3.HC, CHA.9.547.3.LC, CHA.9.547.3.H1, CHA.9.547.3.H2, CHA.9.547.3.H3; CHA.9.547.3.H4, CHA.9.547.3.H4(S241P), CHA.9.547.3.vhCDR1, CHA. 9.547.3.vhCDR2, CHA.9.547. 3.vhCDR3, CHA.9.547.3.vlCDR1, CHA.9.547.3.vlCDR2 and CHA.9.547.3.vhCDR3;

CHA.9.547.4, CHA.9.547.4VH, CHA.9.547.4.VL, CHA.9.547.4.HC, CHA. 9.547.4.LC, CHA.9.547.4.H1, CHA.9.547.4.H2, CHA.9.547.4.H3; CHA.9.547.4.H4, CHA.9.547.4.H4(S241P), CHA.9.547.4.vhCDR1, CHA.9.547.4.vhCDR2, CHA.9.547.4.vhCDR3, CHA.9.547.4.vlCDR1, CHA.9.547.4.vlCDR2 and CHA.9.547.4.vhCDR3;

CHA.9.547.6, CHA.9.547.6 VH, CHA.9.547.6.VL, CHA.9.547.6.HC, CHA.9.547.6.LC, CHA.9.547.6.H1, CHA.9.547.6.H2, CHA.9.547.6.H3; CHA.9.547.6.H4, CHA.9.547.6.H4(S241P), CHA.9.547.6.vhCDR1, CHA.9.547.6.vhCDR2, CHA.9.547. 6.vhCDR3, CHA.9.547.6.vlCDR1, CHA.9.547.6.vlCDR2 and CHA.9.547.6.vhCDR3;

CHA.9.547.7, CHA.9.547.7VH, CHA.9.547.7.VL, CHA.9.547.7.HC, CHA.9.547.7.LC, CHA.9.547.7.H1, CHA.9.547.7.H2, CHA.9.547.7.H3; CHA.9.547.7.H4, CHA.9.547.7.H4(S241P), CHA.9.547.7.vhCDR1, CHA.9.547.7.vhCDR2, CHA.9.547. 7.vhCDR3, CHA.9.547.7.vlCDR1, CHA.9.547.7.vlCDR2 and CHA.9.547.7.vhCDR3;

CHA.9.547.8, CHA.9.547.8VH, CHA.9.547.8.VL, CHA.9.547.8.HC, CHA.9.547.8.LC, CHA.9.547.8.H1, CHA.9.547.8.H2, CHA.9.547.8.H3; CHA.9.547.8.H4, CHA.9.547.8.H4(S241P), CHA.9.547.8.vhCDR1, CHA.9.547.8.vhCDR2, CHA.9.547. 8.vhCDR3, CHA.9.547.8.vlCDR1, CHA.9.547.8.vlCDR2 and CHA.9.547.8.vhCDR3;

CHA.9.547.9, CHA.9.547.9, CHA.9.547.9VH, CHA.9.547.9.VL, CHA.9. 547.9.HC, CHA.9.547.9.LC, CHA.9.547.9.H1, CHA.9.547.9.H2, CHA.9.547.9.H3; CHA.9.547.9.H4, CHA.9.547.9.H4, CHA.9.547.9.H4(S241P), CHA.9.547.9.H4(S241P), CHA.9.547.9.vhCDR1, CHA.9.547.9.vhCDR2, CHA.9.547.9.vhCDR3, CHA.9.547.9.vlCDR1, CHA.9.547.9.vlCDR2 and CHA.9.547.9.vhCDR3;

CHA.9.547.13, CHA.9.547.13, CHA.9.547.13VH, CHA.9.547.13.VL, CHA.9.547.13.HC, CHA. 9.547.13.LC, CHA. 9.547.13.H1, CHA.9.547.13.H2, CHA.9. 547.13.H3; CHA.9.547.13.H4, CHA.9.547.13.H4, CHA.9.547.13.H4(S241P), CHA.9.547.13.H4(S241P), CHA.9.547.13.vhCDR1, CHA.9.547.13.vhCDR2, CHA.9.547. 13.vhCDR3, CHA.9.547.13.vlCDR1, CHA.9.547.13.vlCDR2 and CHA.9.547.13.vhCDR3;

CHA.9.541.1, CHA.9.541.1.VH, CHA.9.541.1.VL, CHA.9.541.1.HC, CHA.9.541.1.LC, CHA.9.541.1.H1, CHA.9.541.1.H2, CHA.9.541.1.H3; CHA.9.541.1.H4, CHA.9.541.1.H4(S241P), CHA.9.541.1.vhCDR1, CHA.9.541.1.vhCDR2, CHA.9.541.1.vhCDR3, CHA.9.541.1.vlCDR1, CHA.9.541.1.vlCDR2 and CHA. 9.541.1.vhCDR3;

CHA.9.541.3, CHA.9.541.3.VH, CHA.9.541.3.VL, CHA.9.541.3.HC, CHA.9.541.3.LC, CHA.9.541.3.H1, CHA.9.541.3.H2, CHA.9.541.3.H3; CHA.9.541.3.H4, CHA.9.541.3.H4(S241P), CHA.9.541.3.vhCDR1, CHA.9.541.3.vhCDR2, CHA.9.541. 3.vhCDR3, CHA.9.541.3.vlCDR1, CHA.9.541.3.vlCDR2 and CHA. 9.541.3.vhCDR3;

CHA.9.541.4, CHA.9.541.4.VH, CHA.9.541.4.VL, CHA.9.541.4.HC, CHA.9.541.4.LC, CHA.9.541.4.H1, CHA.9.541.4.H2, CHA.9.541.4.H3; CHA.9.541.4.H4, CHA.9.541.4.H4(S241P), CHA.9.541.4.vhCDR1, CHA.9.541.4.vhCDR2, CHA.9.541. 4.vhCDR3, CHA.9.541.4.vlCDR1, CHA.9.541.4.vlCDR2 and CHA. 9.541.4.vhCDR3;

CHA.9.541.5, CHA.9.541.5.VH, CHA.9.541.5.VL, CHA.9.541.5.HC, CHA.9.541.5.LC, CHA.9.541.5.H1, CHA.9.541.5.H2, CHA.9.541.5.H3; CHA.9.541.5.H4, CHA.9.541.5.H4(S241P), CHA.9.541.5.vhCDR1, CHA.9.541.5.vhCDR2, CHA.9.541. 5.vhCDR3, CHA.9.541.5.vlCDR1, CHA.9.541.5.vlCDR2 and CHA. 9.541.5.vhCDR3;

CHA.9.541.6, CHA.9.541.6.VH, CHA.9.541.6.VL, CHA.9.541.6.HC, CHA.9.541.6.LC, CHA.9.541.6.H1, CHA.9.541.6.H2, CHA.9.541.6.H3; CHA.9.541.6.H4, CHA.9.541.6.H4(S241P), CHA.9.541.6.vhCDR1, CHA.9.541.6.vhCDR2, CHA.9.541. 6.vhCDR3, CHA.9.541.6.vlCDR1, CHA.9.541.6.vlCDR2 and CHA.9.541.6.vhCDR3;

CHA.9.541.7, CHA.9.541.7.VH, CHA.9.541.7.VL, CHA.9.541.7.HC, CHA.9.541.7.LC, CHA.9.541.7.H1, CHA.9.541.7.H2, CHA.9.541.7.H3; CHA.9.541.7.H4, CHA.9.541.7.H4(S241P), CHA.9.541.7.vhCDR1, CHA.9.541.7.vhCDR2, CHA.9.541. 7.vhCDR3, CHA.9.541.7.vlCDR1, CHA.9.541.7.vlCDR2 and CHA.9.541.7.vhCDR3; and

CHA.9.541.8, CHA.9.541.8.VH, CHA.9.541.8.VL, CHA.9.541.8.HC, CHA.9.541.8.LC, CHA.9.541.8.H1, CHA.9.541.8.H2, CHA.9.541.8.H3; CHA.9.541.8.H4, CHA.9.541.8.H4(S241P); CHA.9.541.8vhCDR1, CHA.9.541.8.vhCDR2, CHA.9.541. 8.vhCDR3, CHA.9.541.8.vlCDR1, CHA.9.541.8.vlCDR2 and CHA.9.541.8.vhCDR3.

CHA.9.547.18vhCDR1, CHA.9.547.18.vhCDR2, CHA.9.547.18.vhCDR3, CHA.9.547.18.vlCDR1, CHA.9.547.18vlCDR2, and CHA.9.547.18.vlCDR3.

In the case of scFvs comprising the CDRs of the antibodies above, these are labeled as scFvs that include a scFv comprising a variable heavy domain with the vhCDRs, a linker and a variable light domain with the vlCDRs, again as above in either orientation. Thus the invention includes scFv-CHA.9.536.3.1, scFv-CHA.9.536.3, scFv-CHA.9.536.4, scFv-CHA.9.536.5, scFv-CHA.9.536.7, scFv-CHA.9.536.8, scFv-CHA.9.560.1, scFv-CHA.9.560.3, scFv-CHA.9.560.4, scFv-CHA.9.560.5, scFv-CHA.9.560.6, scFv-CHA.9.560.7, scFv-CHA.9.560.8, scFv-CHA.9.546.1, scFv-CHA.9.547.1, scFv-CHA.9.547.2, scFv-CHA.9.547.3, scFv-CHA.9.547.4, scFv-CHA.9.547.6, scFv-CHA.9.547.7, scFv-CHA.9.547.8, scFv-CHA.9.547.9, scFv-CHA.9.547.13, scFv-CHA.9.541.1, scFv-CHA.9.541.3, scFv-CHA.9.541.4, scFv-CHA.9.541.5, scFv-CHA.9.541.6, scFv-CHA.9.541.7 and scFv-CHA.9.541.8.

In addition, CHA.9.543 binds to TIGIT but does not block the TIGIT-PVR interaction.

As discussed herein, the invention further provides variants of the above components (CPA and CHA), including variants in the CDRs, as outlined above. Thus, the invention provides antibodies comprising a set of 6 CDRs as outlined herein that can contain one, two or three amino acid differences in the set of CDRs, as long as the antibody still binds to TIGIT. Suitable assays for testing whether an anti-TIGIT antibody that contains mutations as compared to the CDR sequences outlined herein are known in the art, such as Biacore assays.

In addition, the invention further provides variants of the above variable heavy and light chains. In this case, the variable heavy chains can be 80%, 90%, 95%, 98% or 99% identical to the “VH” sequences herein, and/or contain from 1, 2, 3, 4, 5, 6, 7, 8, 9, 10 amino acid changes, or more, when Fc variants are used. Variable light chains are provided that can be 80%, 90%, 95%, 98% or 99% identical to the “VL” sequences herein (and in particular CPA.9.086), and/or contain from 1, 2, 3, 4, 5, 6, 7, 8, 9, 10 amino acid changes, or more, when Fc variants are used. In these embodiments, the invention includes these variants as long as the anti-TIGIT antibody still binds to TIGIT. Suitable assays for testing whether an anti-TIGIT antibody that contains mutations as compared to the CDR sequences outlined herein are known in the art, such as Biacore assays.

Similarly, heavy and light chains are provided that are 80%, 90%, 95%, 98% or 99% identical to the full length “HC” and “LC” sequences herein (and in particular CPA.9.086), and/or contain from 1, 2, 3, 4, 5, 6, 7, 8, 9, 10 amino acid changes, or more, when Fc variants are used. In these embodiments, the invention includes these variants as long as the anti-TIGIT antibody still binds to TIGIT. Suitable assays for testing whether an anti-TIGIT antibody that contains mutations as compared to the CDR sequences outlined herein are known in the art, such as Biacore assays.

In addition, the framework regions of the variable heavy and variable light chains of either the CPA or CHA antibodies herein can be humanized (or, in the case of the CHA antibodies, “rehumanized”, to the extent that alternative humanization methods can be done) as is known in the art (with occasional variants generated in the CDRs as needed), and thus humanized variants of the VH and VL chains of FIG. 22A-22D can be generated (and in particular CPA.9.086). Furthermore, the humanized variable heavy and light domains can then be fused with human constant regions, such as the constant regions from IgG1, IgG2, IgG3 and IgG4 (including IgG4(S241P)).

In particular, as is known in the art, murine VH and VL chains can be humanized as is known in the art, for example, using the IgBLAST program of the NCBI website, as outlined in Ye et al. Nucleic Acids Res. 41:W34-W40 (2013), herein incorporated by reference in its entirety for the humanization methods. IgBLAST takes a murine VH and/or VL sequence and compares it to a library of known human germline sequences. As shown herein, for the humanized sequences generated herein, the databases used were IMGT human VH genes (F+ORF, 273 germline sequences) and IMGT human VL kappa genes (F+ORF, 74 germline sequences). An exemplary five CHA sequences were chosen: CHA.9.536, CHA9.560, CHA.9.546, CHA.9.547 and CHA.9.541 (see FIG. 22A-22D). For this embodiment of the humanization, human germline IGHV1-46(allele1) was chosen for all 5 as the acceptor sequence and the human heavy chain IGHJ4(allele1) joining region (J gene). For three of four (CHA.7.518, CHA.7.530, CHA.7.538_1 and CHA.7.538_2), human germline IGKV1-39(allele 1) was chosen as the acceptor sequence and human light chain IGKJ2(allele1) (J gene) was chosen. The J gene was chosen from human joining region sequences compiled at IMGT® the international ImMunoGeneTics information system as www.imgt.org. CDRs were defined according to the AbM definition (see www.bioinfo.org.uk/abs/). In some embodiments, the anti-TIGIT antibodies for use with the present invention include TIGIT binding portions or antigen binding domains wherein the V_{H} and V_{L} sequences of different TIGIT binding portions or antigen binding domains can be “mixed and matched” to create other TIGIT binding portions or antigen binding domains. TIGIT binding of such “mixed and matched” anti-TIGIT antibodies can be tested using the binding assays described above. e.g., ELISAs or Biacore assays). In some embodiments, when V_{H} and V_{L} chains are mixed and matched, a V_{H} sequence from a particular V_{H}/V_{L} pairing is replaced with a structurally similar V_{H} sequence. Likewise, in some embodiments, a V_{L} sequence from a particular V_{H}/V_{L} pairing is replaced with a structurally similar V_{L} sequence. For example, the V_{H} and V_{L} sequences of homologous antibodies are particularly amenable for mixing and matching.

Accordingly, the anti-TIGIT antibodies for use with the invention comprise CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more amino acid substitutions; (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein. In particular, the anti-TIGIT antibody can comprise the antigen binding domain from the CPA.9.086 antibody which can have sequences selected from (a), (b), (c) or (d). Additionally included in the definition of anti-TIGIT antibodies are antibodies that comprise TIGIT binding domains that share identity to the binding domains from the TIGIT antibodies enumerated herein. That is, in certain embodiments, an anti-TIGIT antibodies according to the invention comprises heavy and light chain variable regions comprising amino acid sequences that are identical to all or part of the binding domains from the anti-TIGIT amino acid sequences of preferred anti-TIGIT antibodies, respectively, wherein the antibodies retain the desired functional properties of the parent anti-TIGIT antibodies. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between TIGIT binding domains or antigen binding domains is at least 75%, at least 80%, at least 90%, with at least about 95%, 96%, 97%, 98% or 99% percent identity being preferred. The percentage identity may be along the whole amino acid sequence, for example the entire heavy or light chain or along a portion of the chains. For example, included within the definition of the anti-TIGIT antibodies for use with the invention are those whose TIGIT binding portion or antigen binding domains shares identity along the entire variable region (for example, where the identity is 95% or 98% identical along the variable regions), or along the entire constant region, or along just the Fc domain. In particular, the invention provides anti-TIGIT antibodies that have TIGIT binding portions or antigen binding domains with at least 75%, at least 80%, at least 90%, with at least about 95%, 96%, 97%, 98%, or 99% percent identity being preferred, with the CPA.9.086 antibody. In particular, the invention provides anti-TIGIT antibodies that have TIGIT binding portions or antigen binding domains with at least 75%, at least 80%, at least 90%, with at least about 95%, 96%, 97%, 98%, or 99% percent identity being preferred, with the CHA.9.547.18 antibody.

In addition, also included are sequences that may have the identical CDRs but changes in the framework portions of the variable domain (or entire heavy or light chain). For example, TIGIT antibodies include those with CDRs identical to those shown in FIG. 22A-22D but whose identity along the variable region can be lower, for example 95 or 98% percent identical. In particular, the invention provides anti-TIGIT antibodies that have TIGIT binding portions or antigen binding domains with identical CDRs to CPA.9.086 but with framework regions that are 95% or 98% identical to CPA.9.086. In particular, the invention provides anti-TIGIT antibodies that have TIGIT binding portions or antigen binding domains with identical CDRs to CHA.9.547.18but with framework regions that are 95% or 98% identical to CHA.9.547.18.

In addition, also included are sequences that may have the identical CDRs but changes in the framework portions of the variable domain (or entire heavy or light chain). For example, TIGIT antibodies include those with CDRs identical to those shown in FIG. 23A-23EE but whose identity along the variable region can be lower, for example 95 or 98% percent identical.

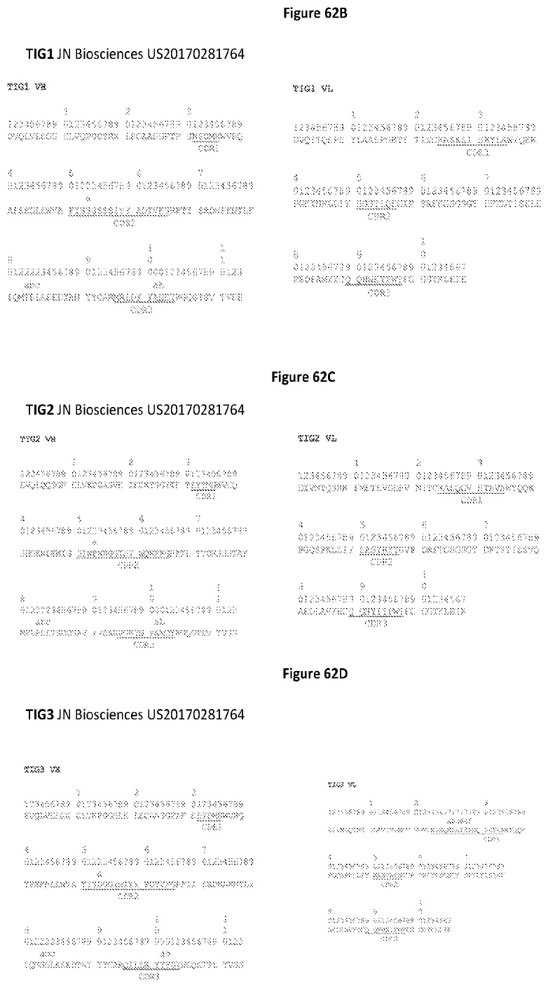

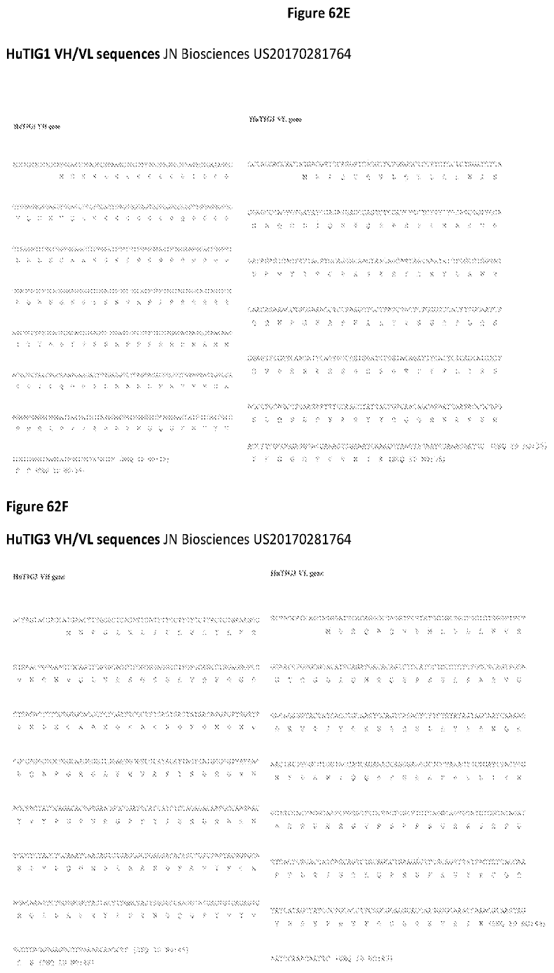

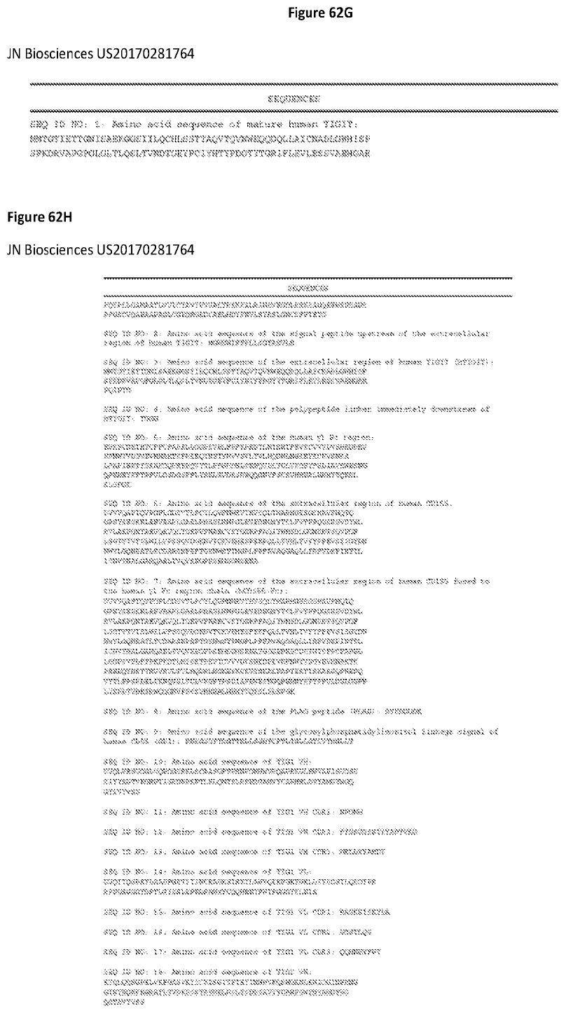

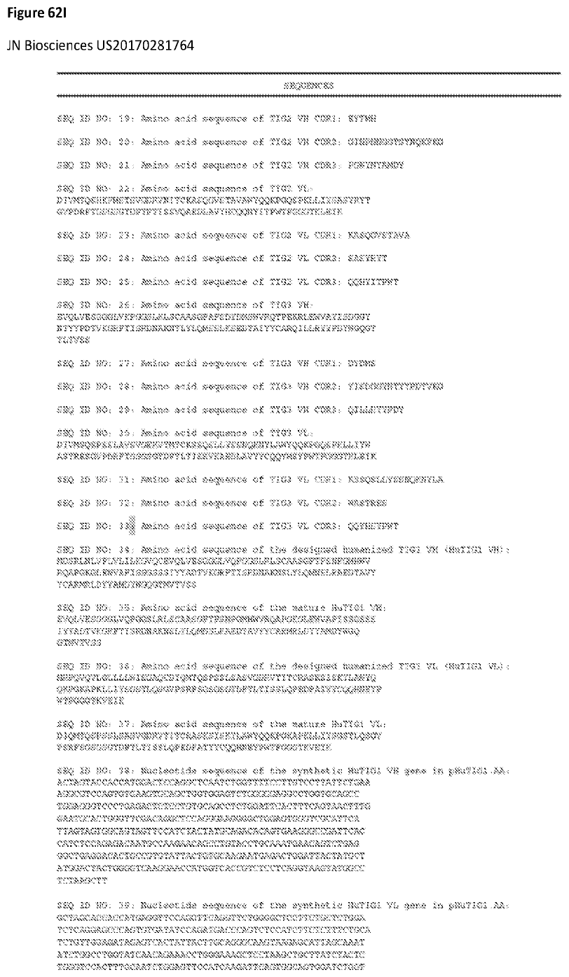

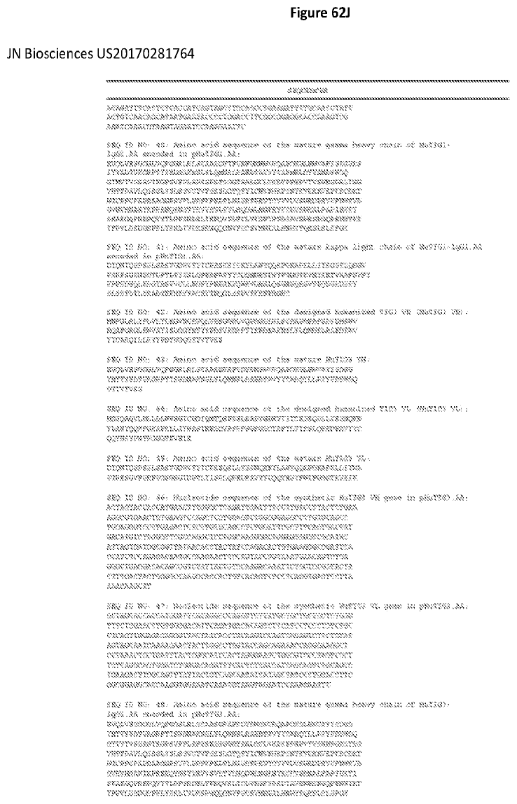

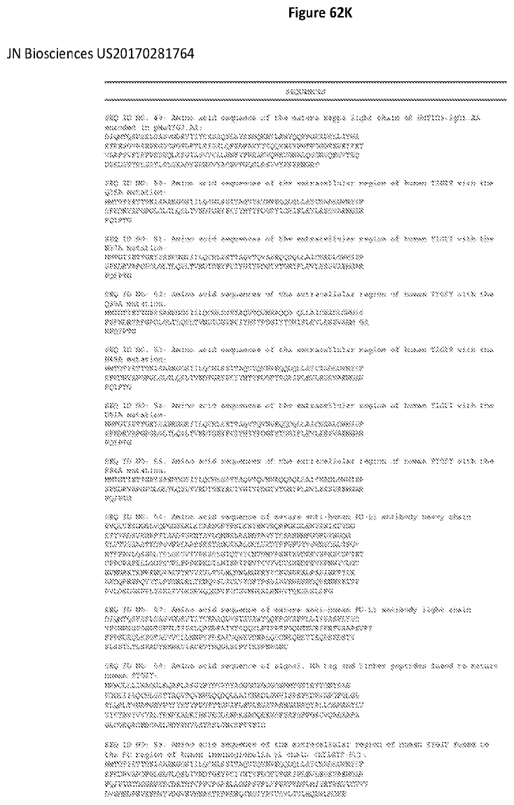

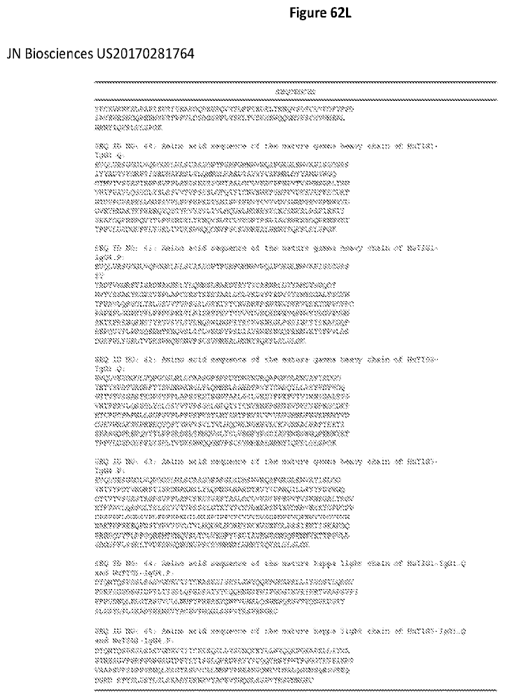

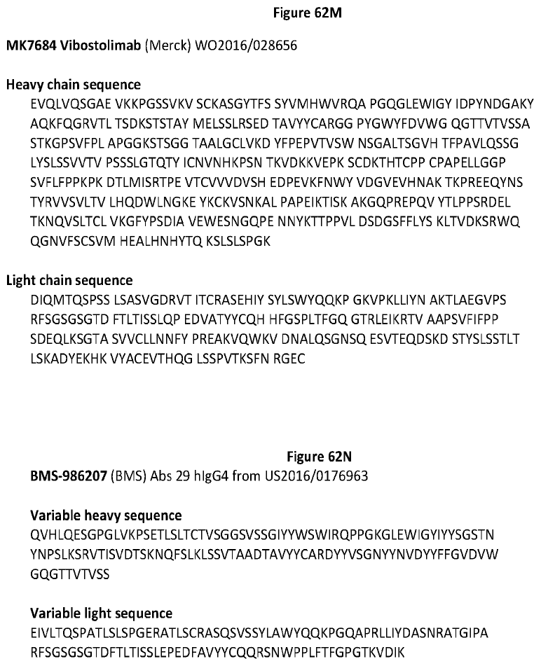

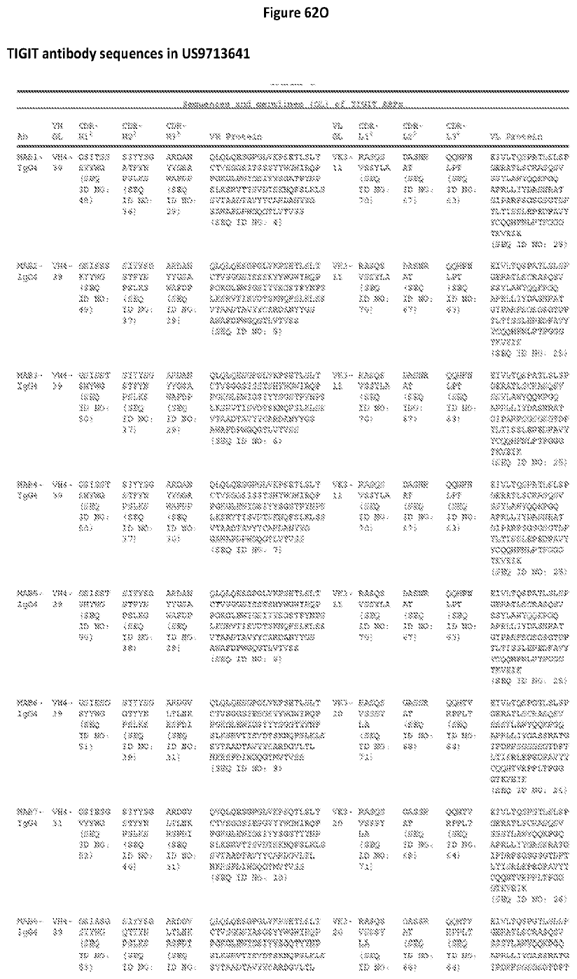

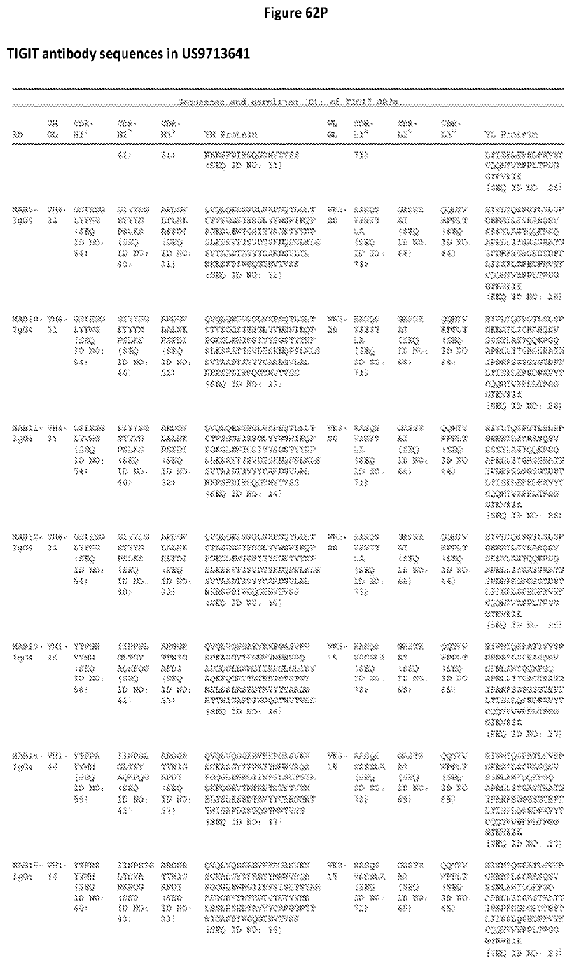

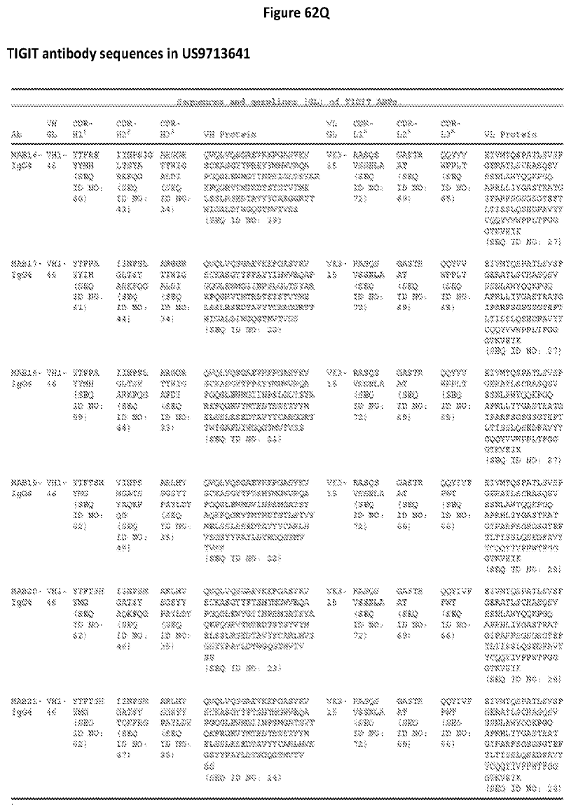

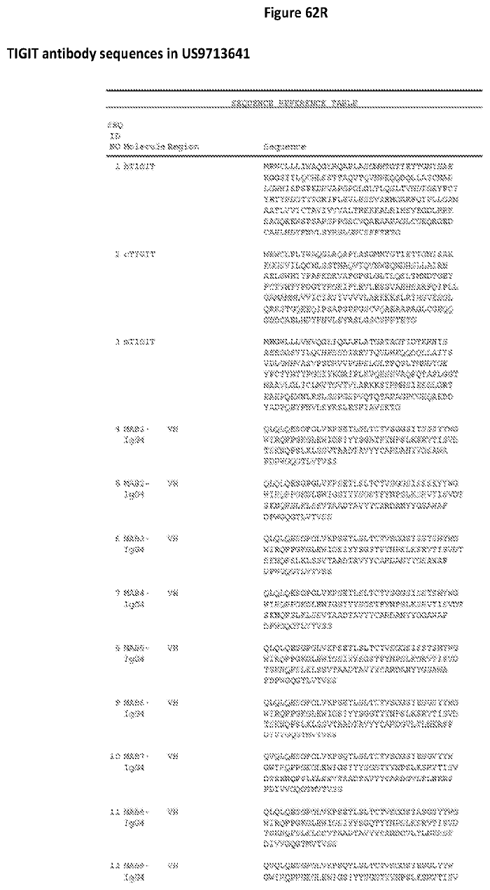

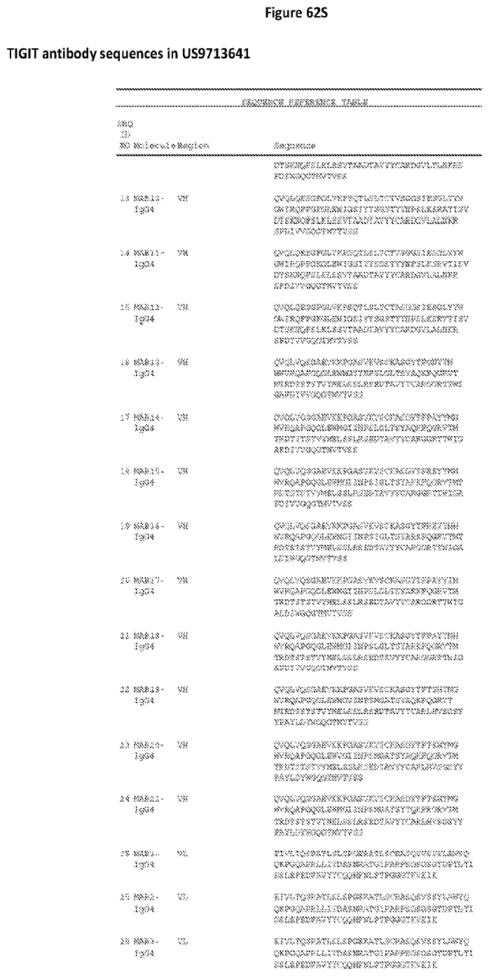

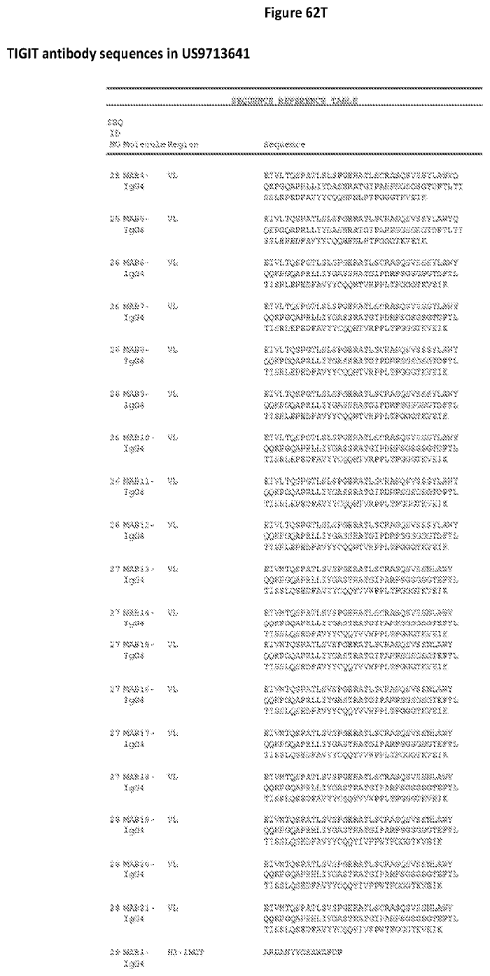

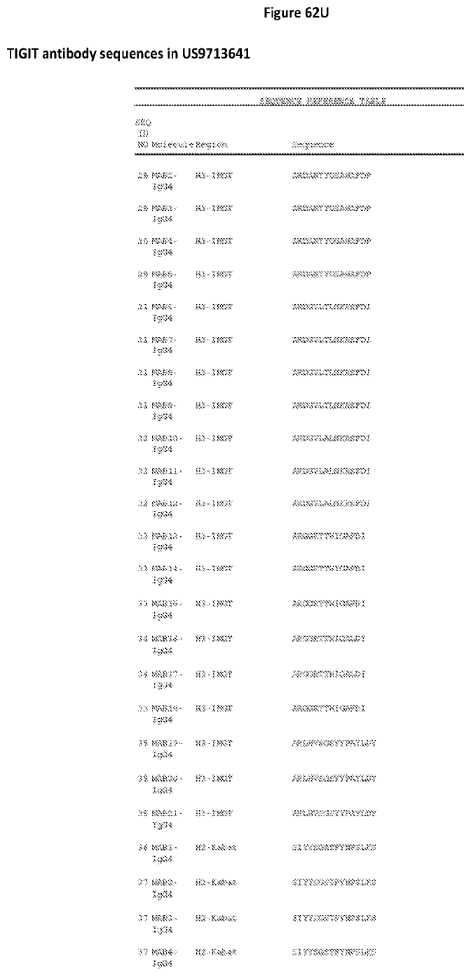

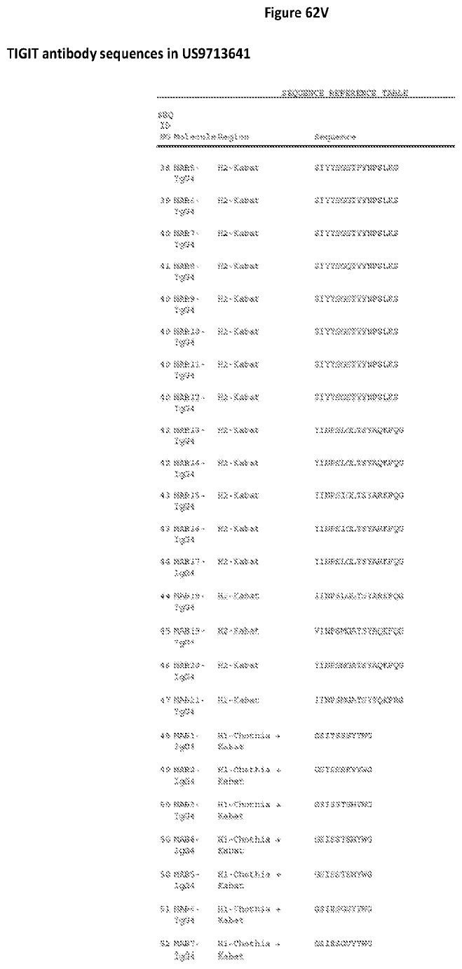

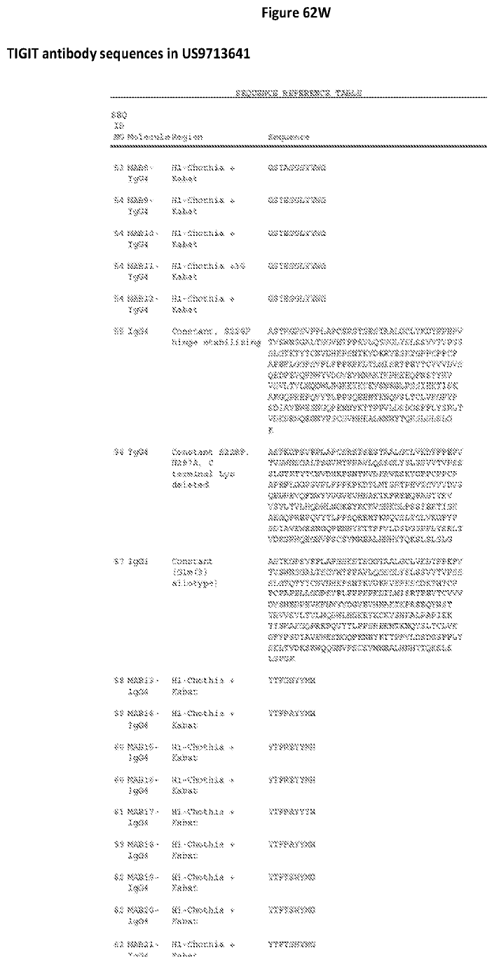

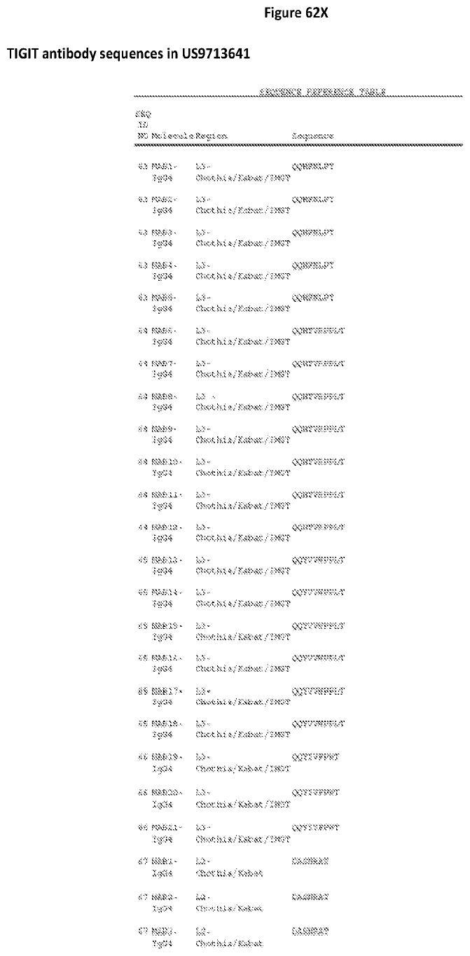

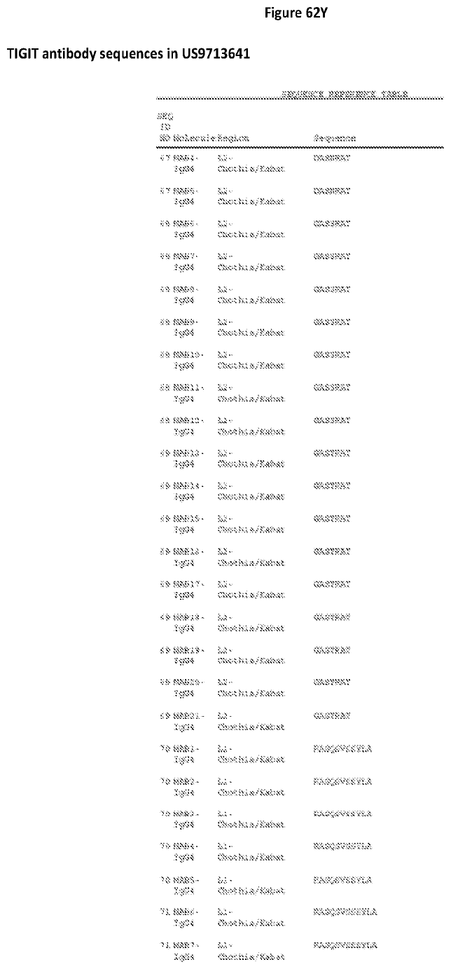

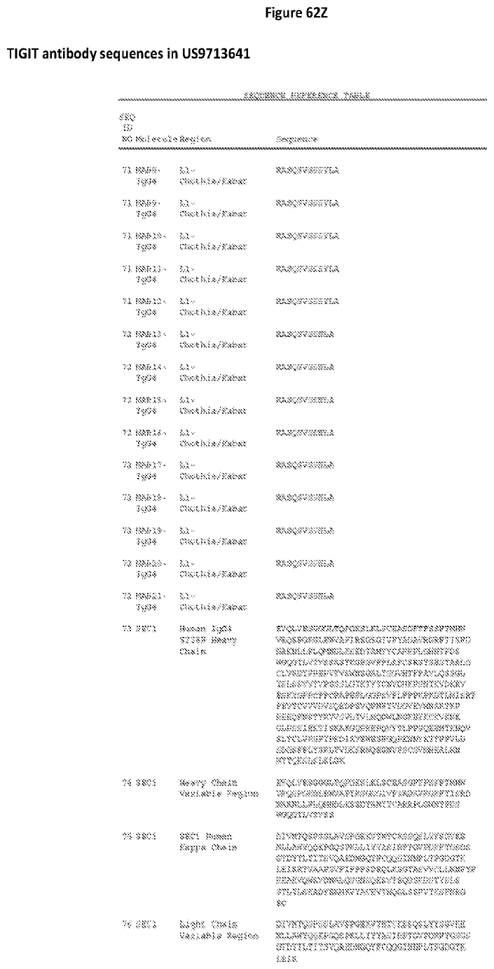

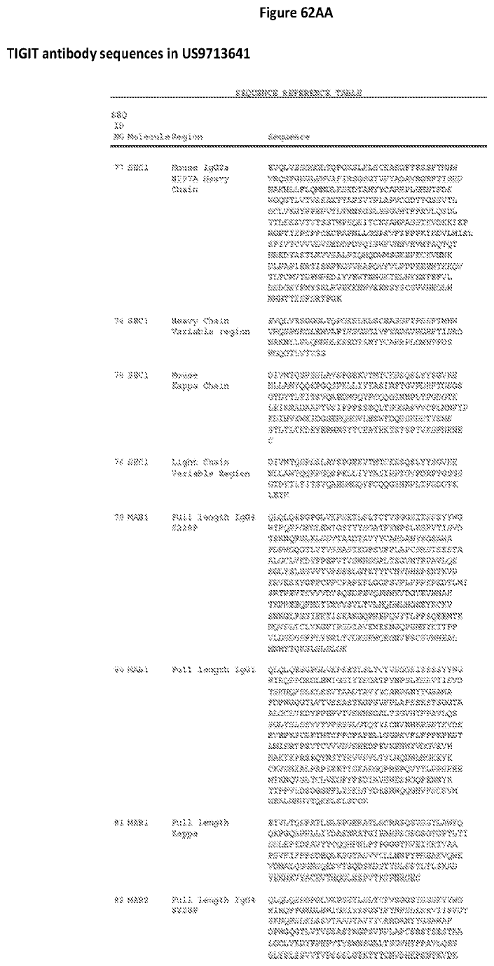

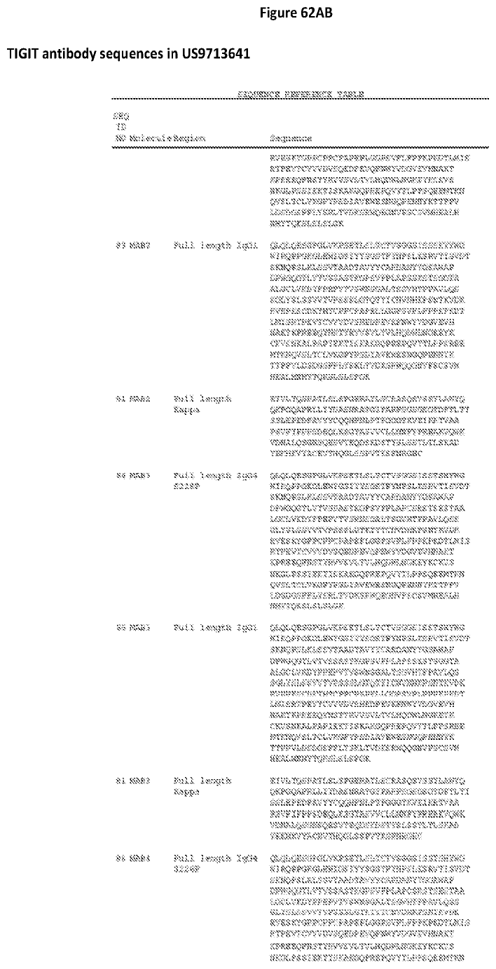

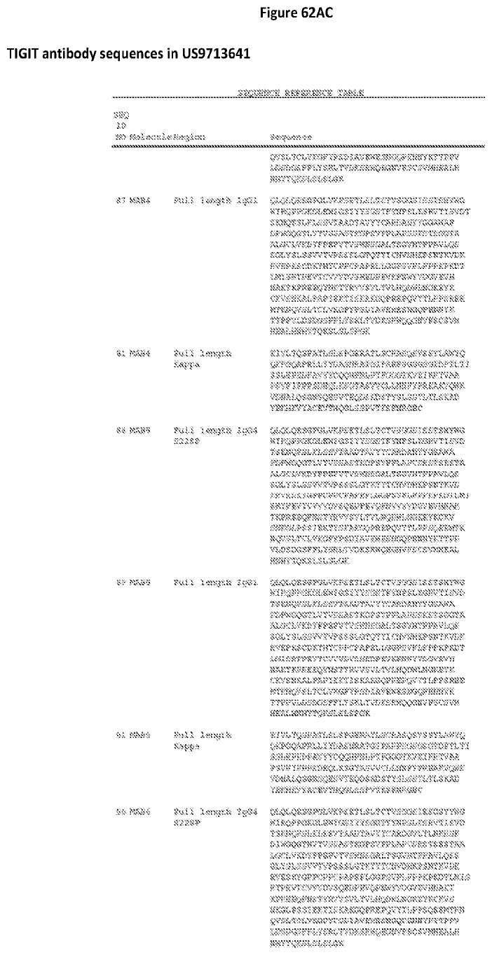

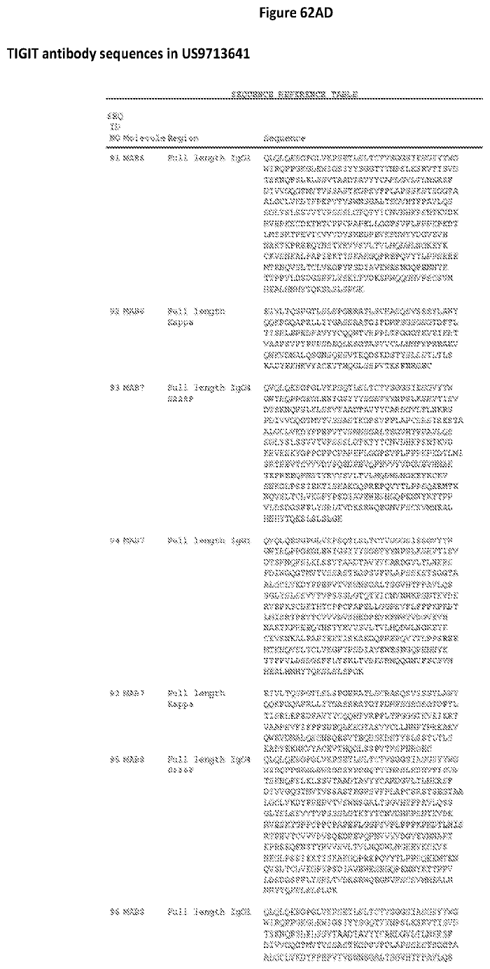

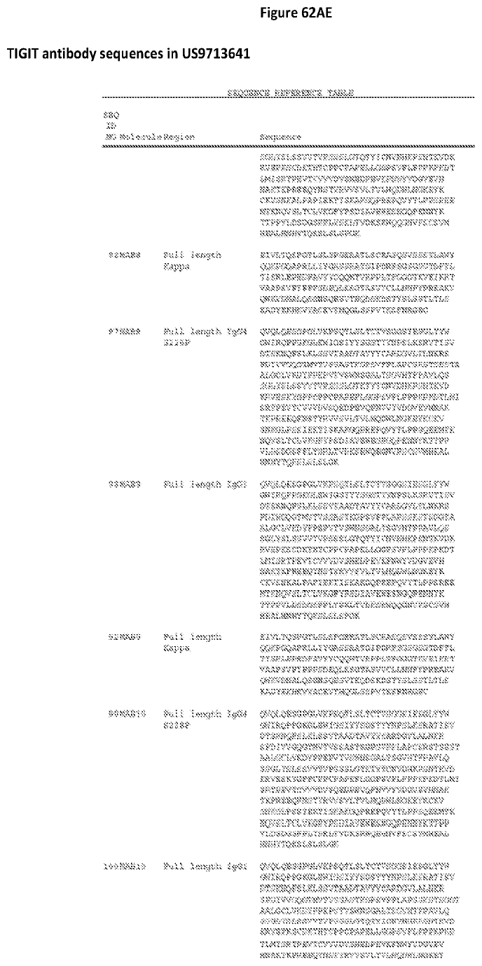

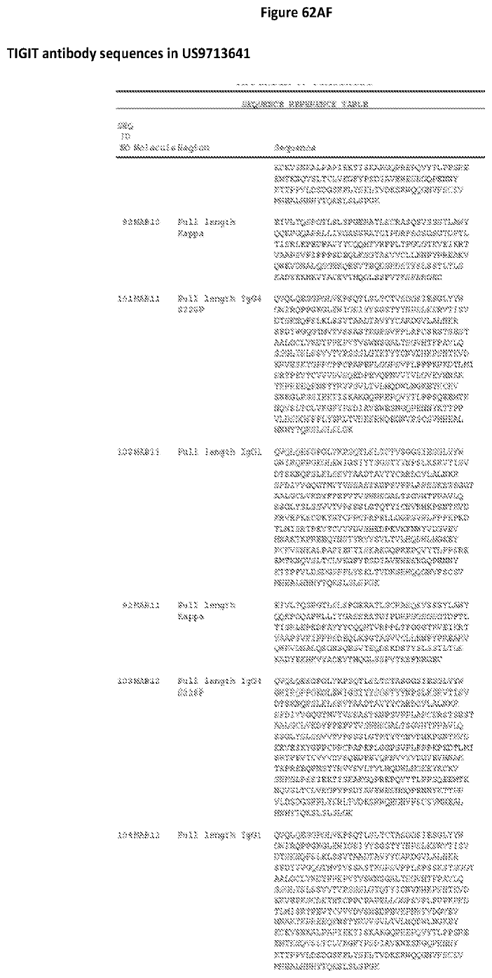

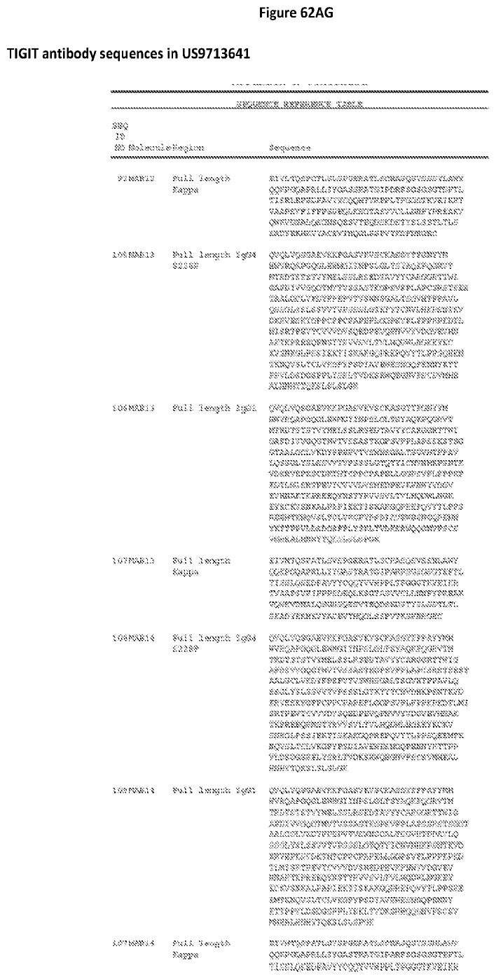

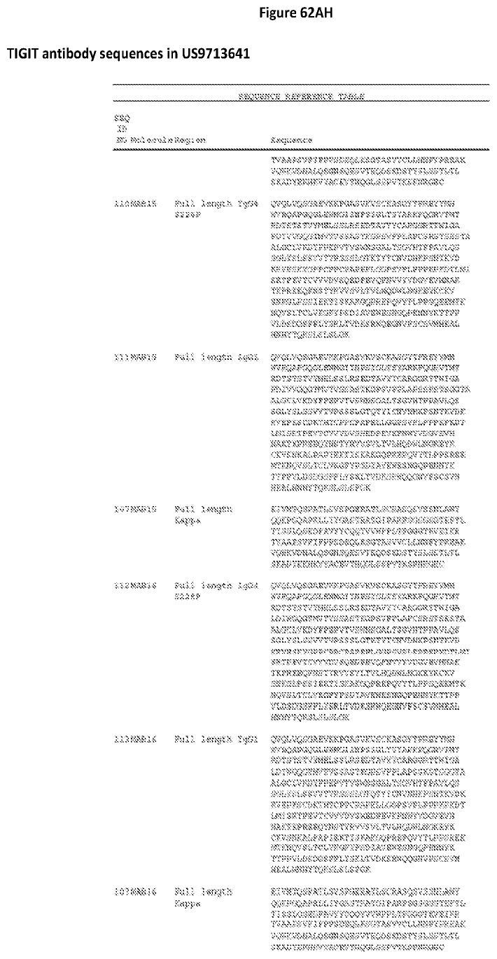

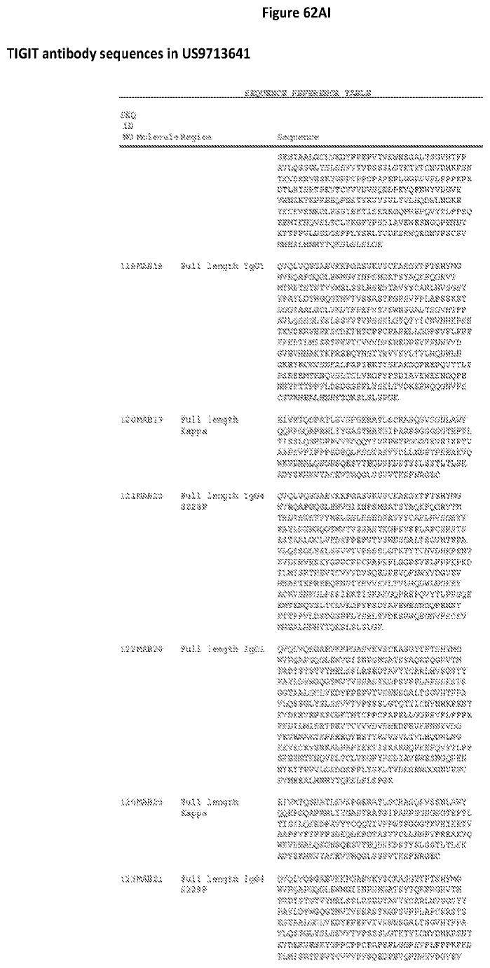

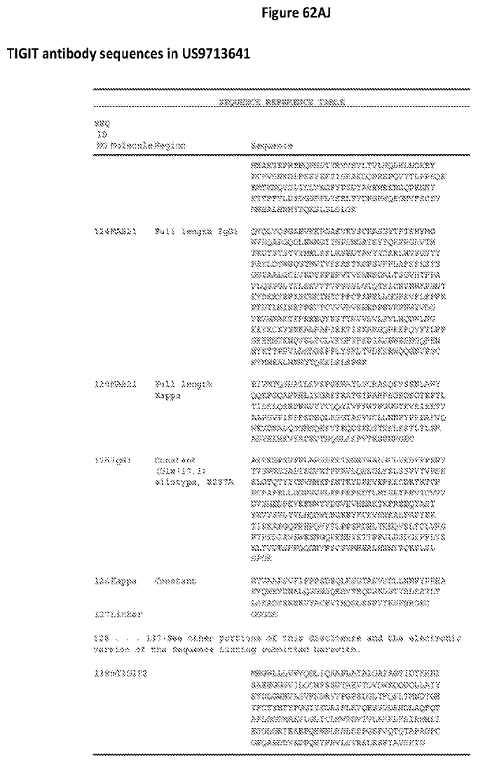

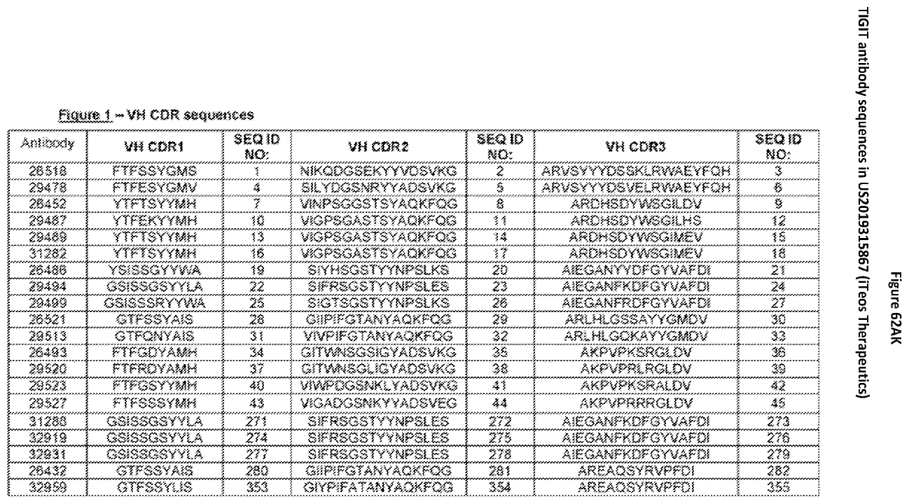

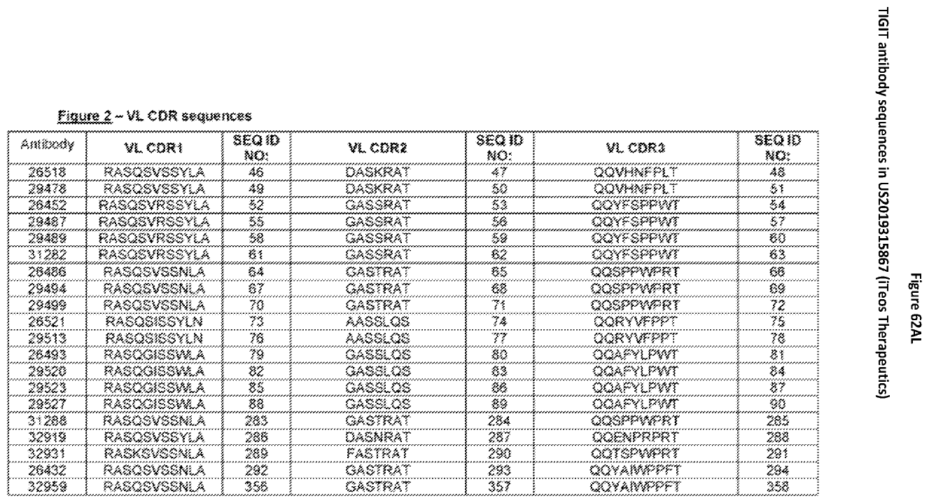

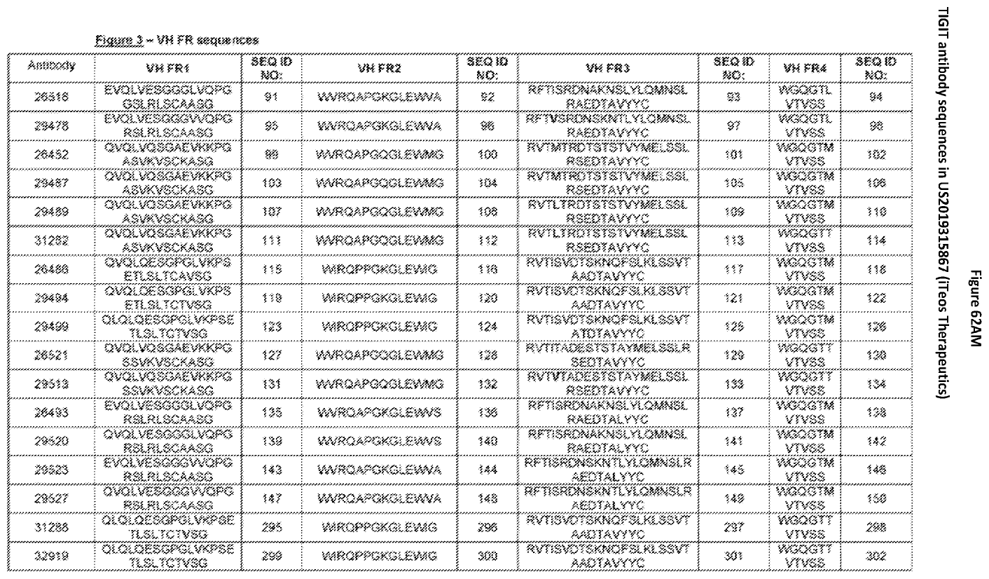

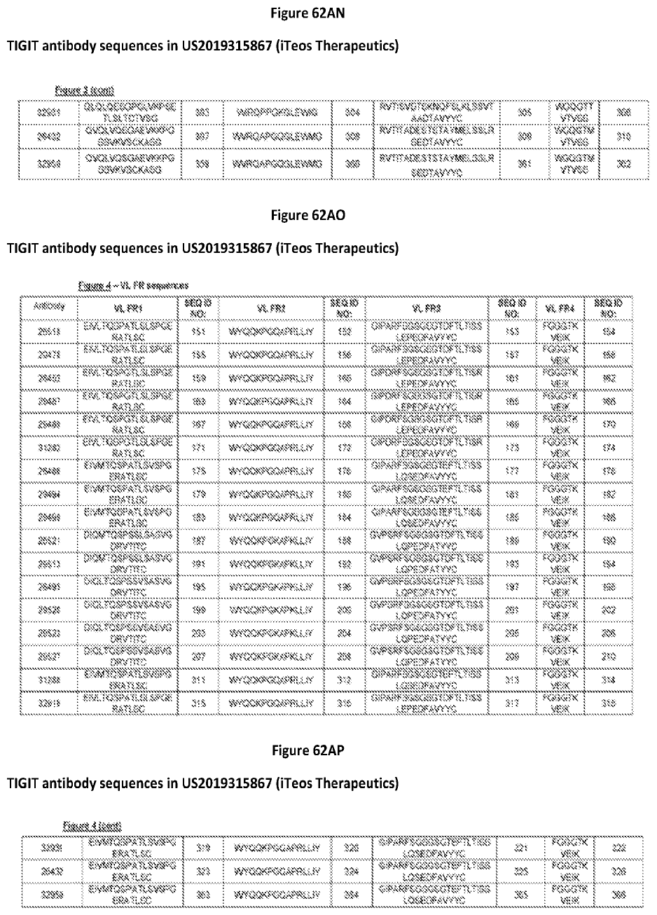

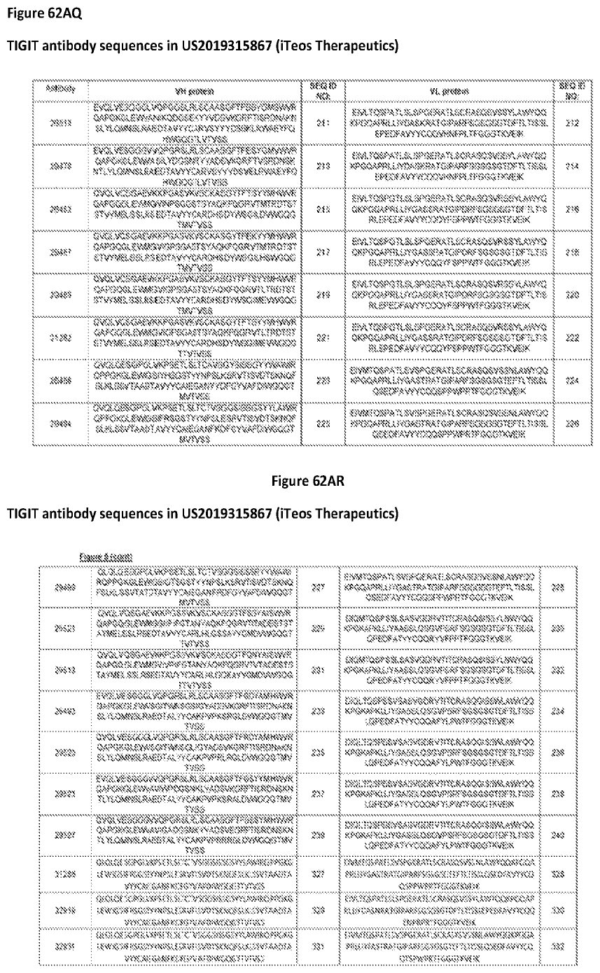

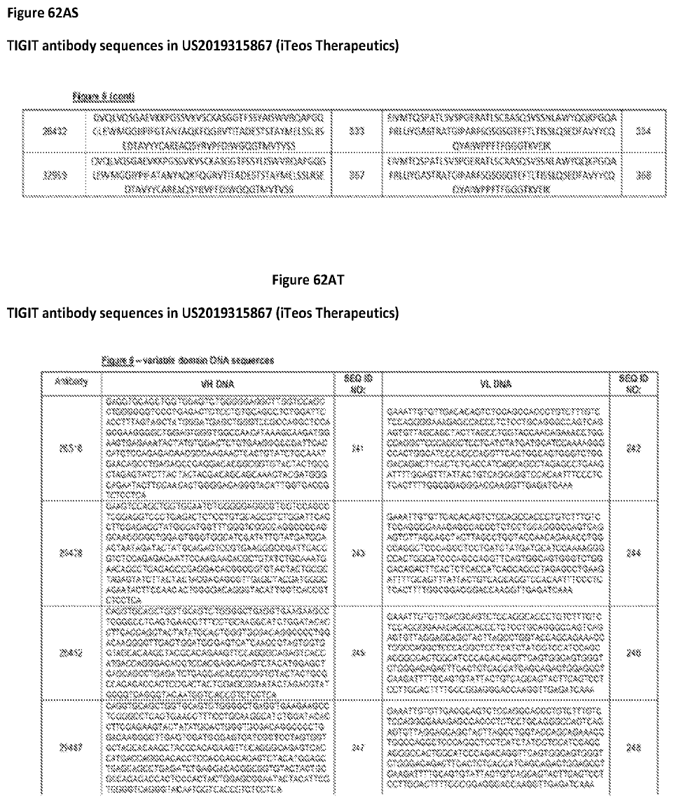

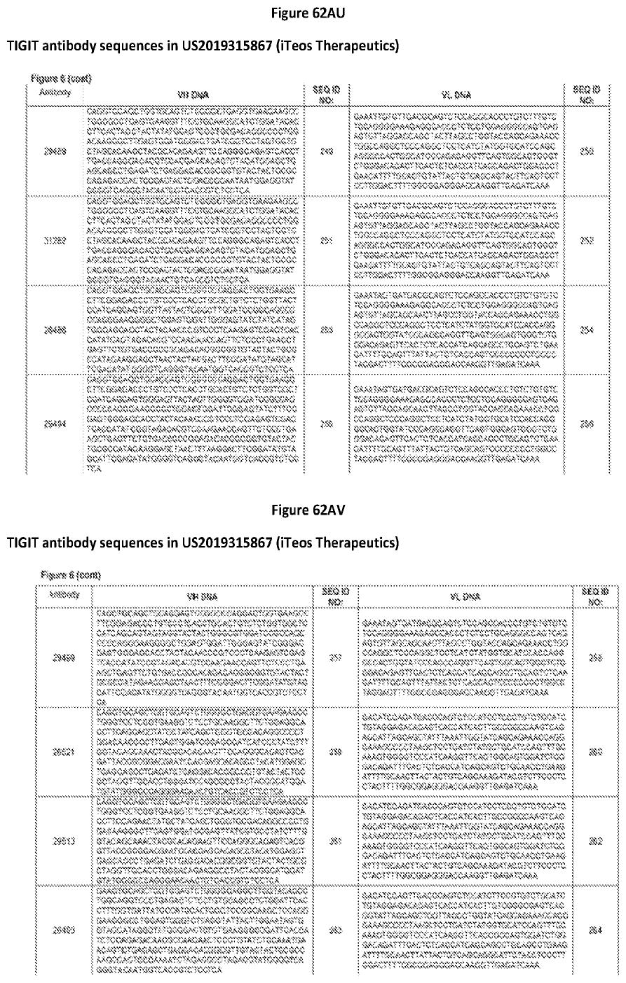

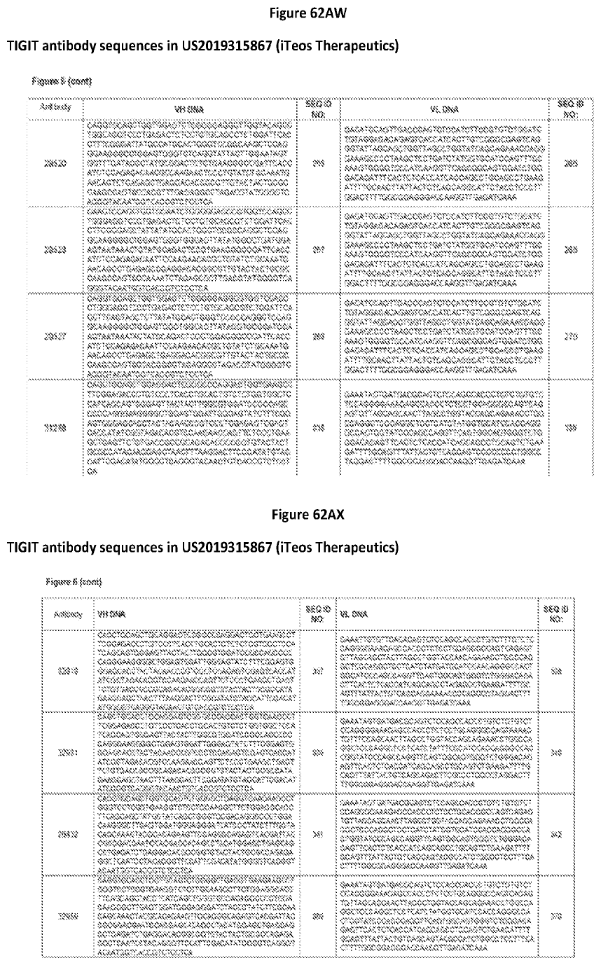

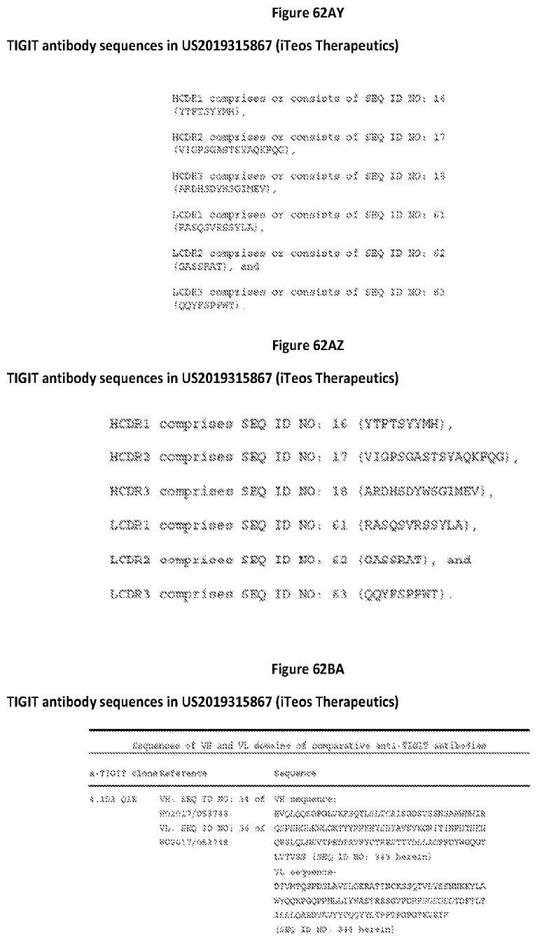

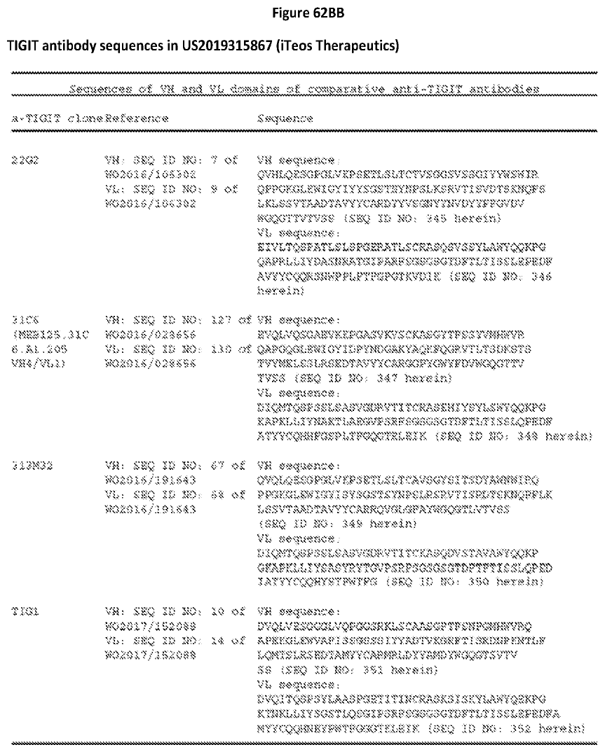

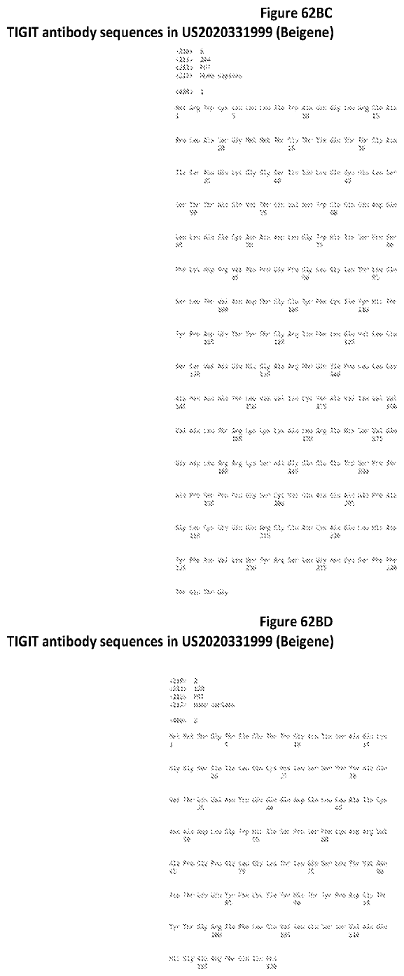

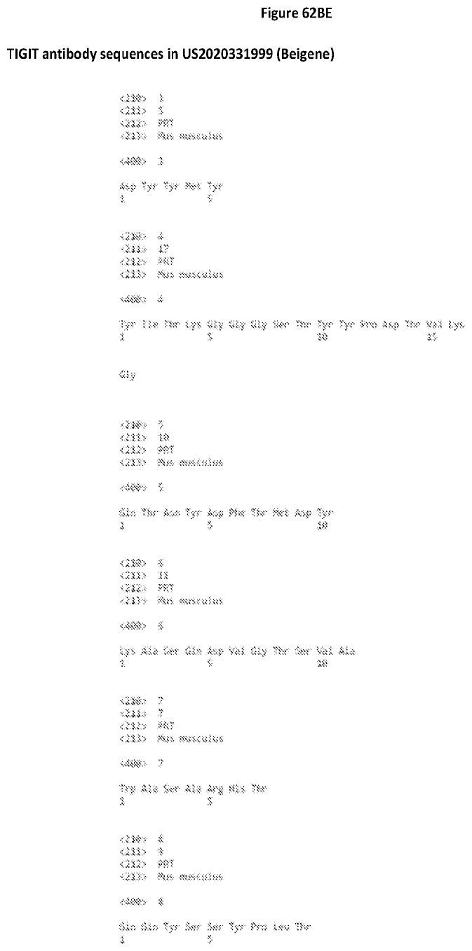

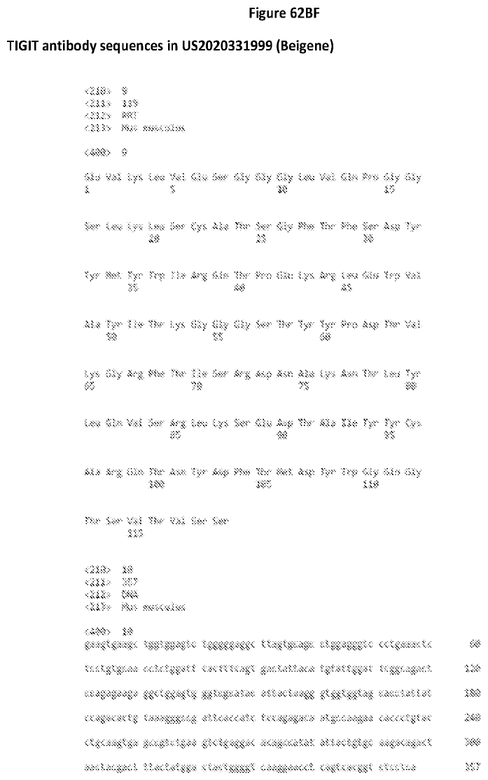

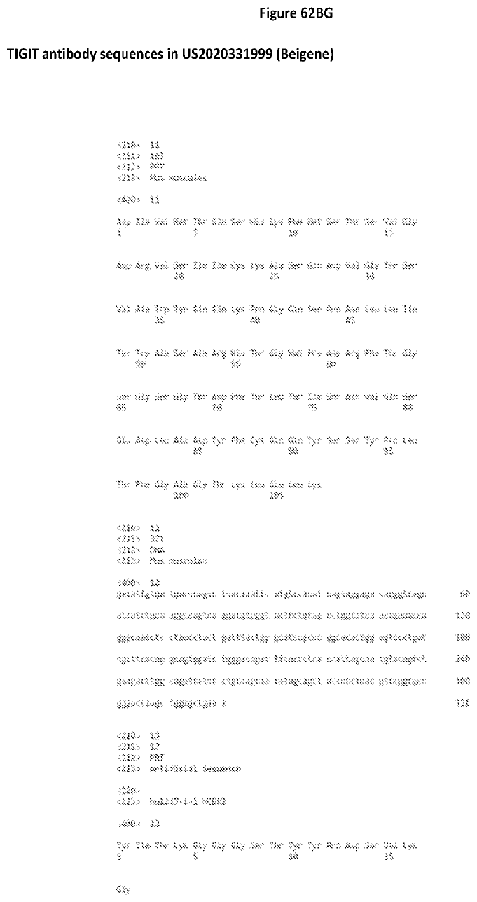

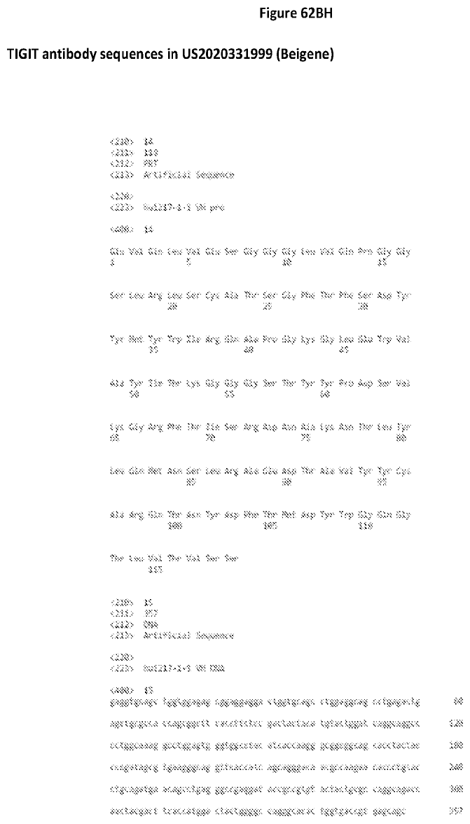

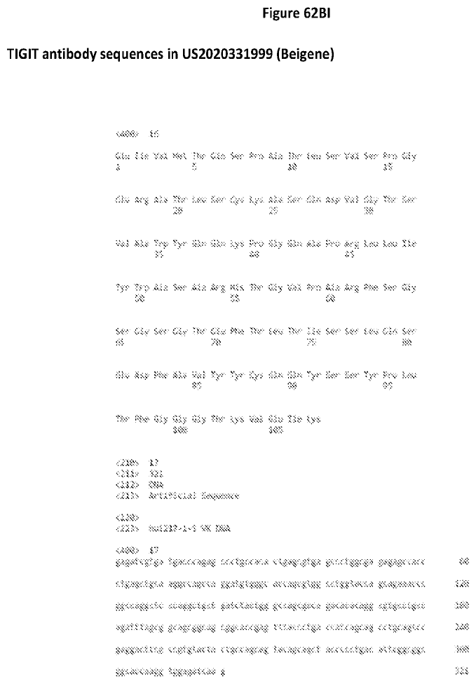

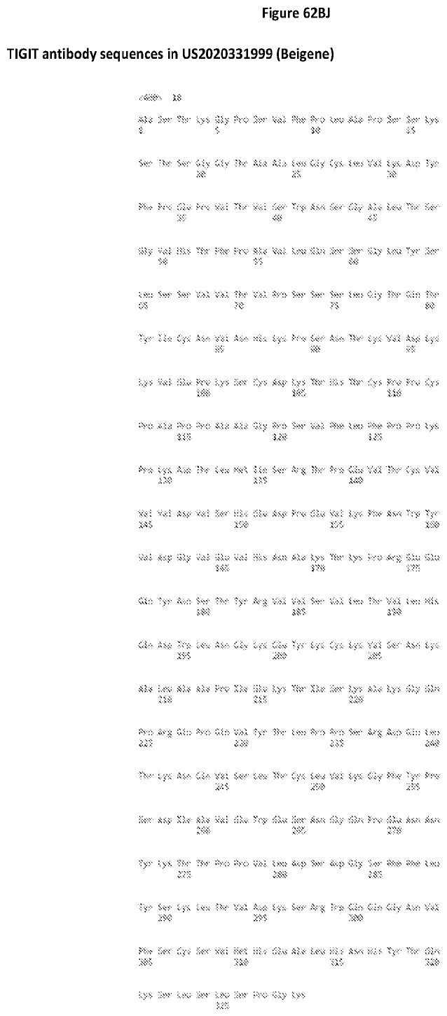

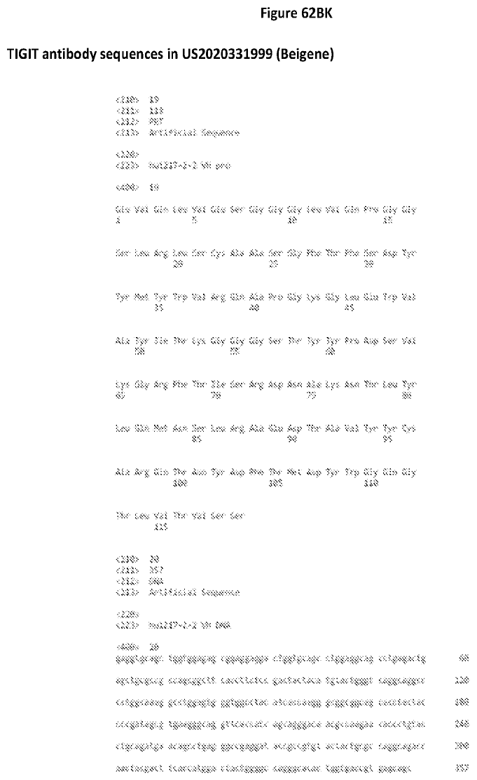

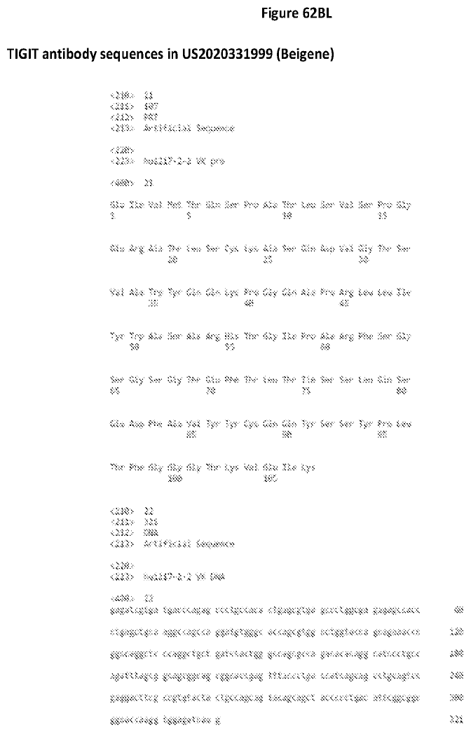

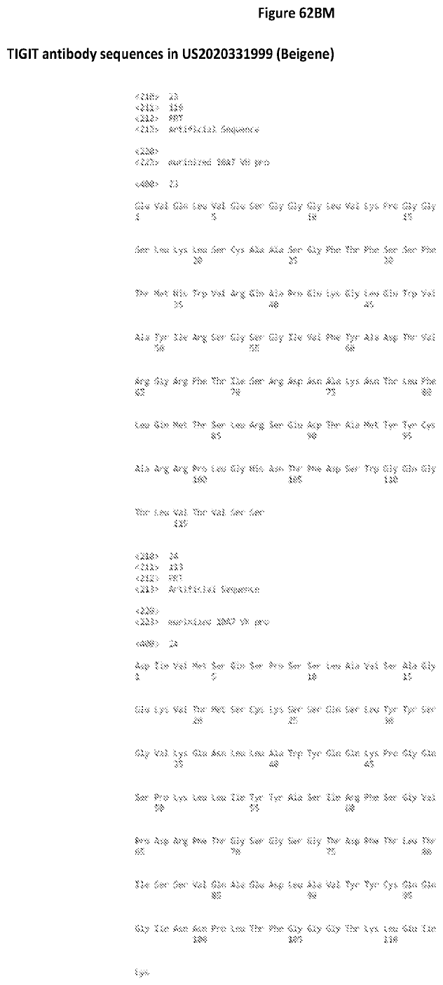

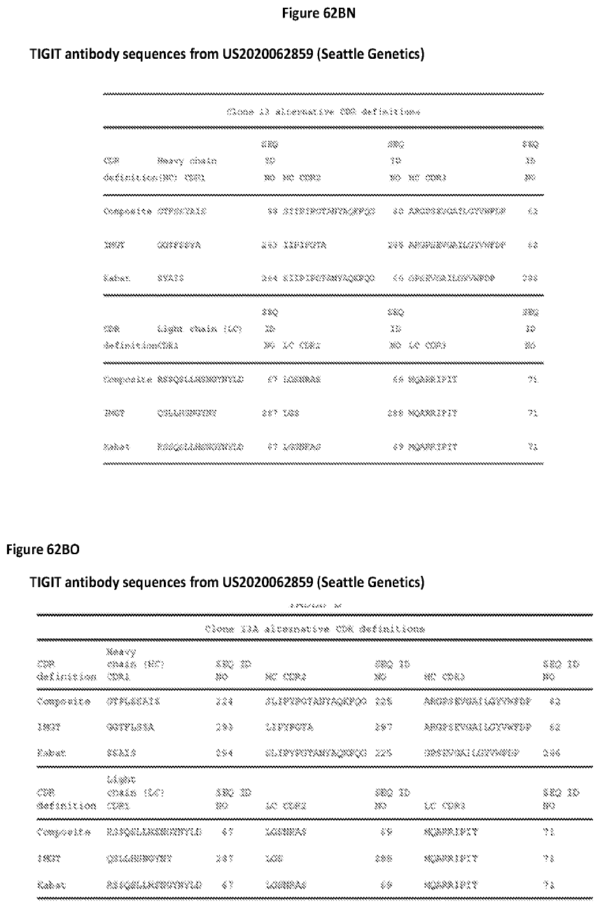

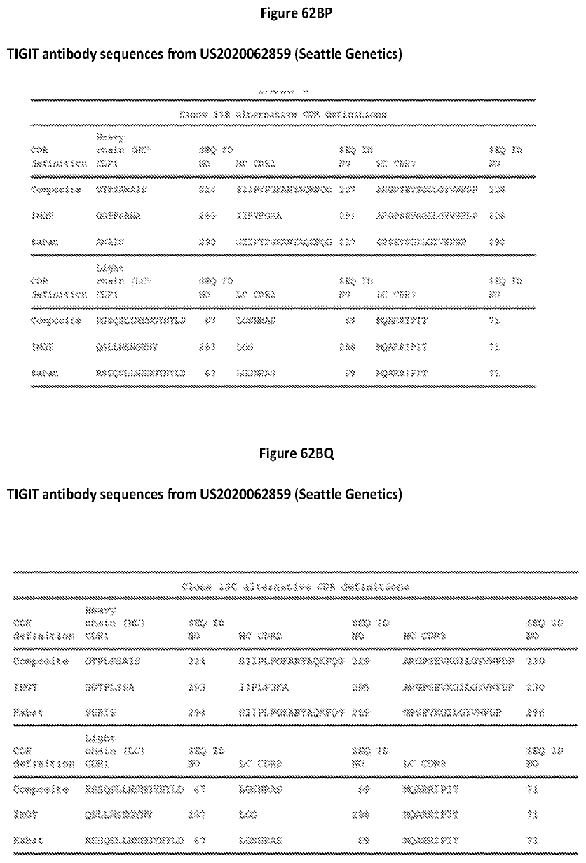

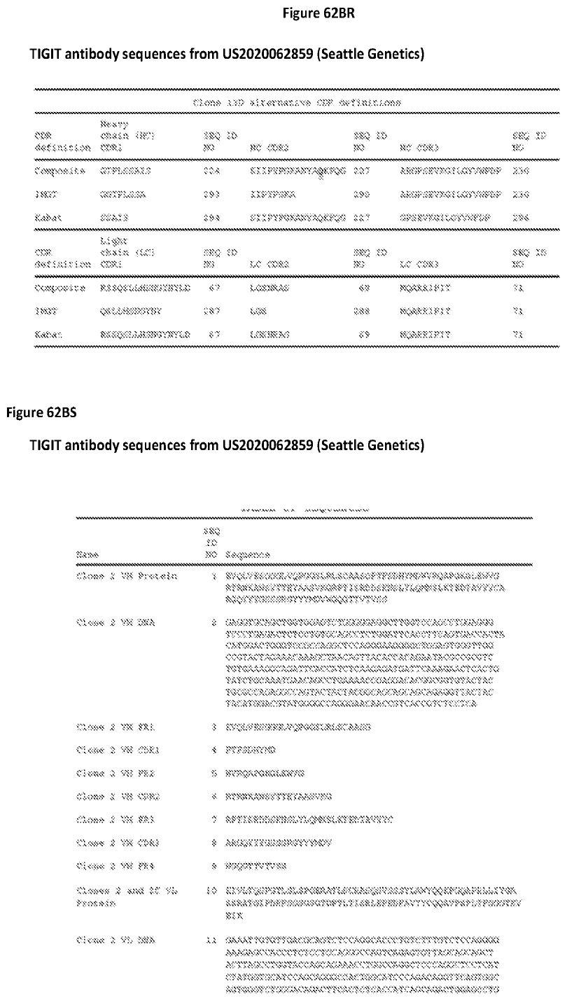

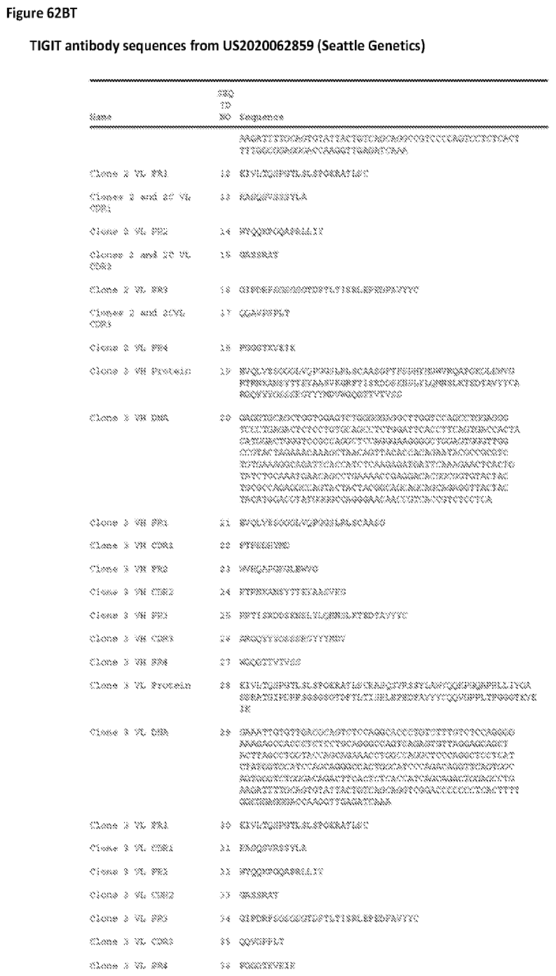

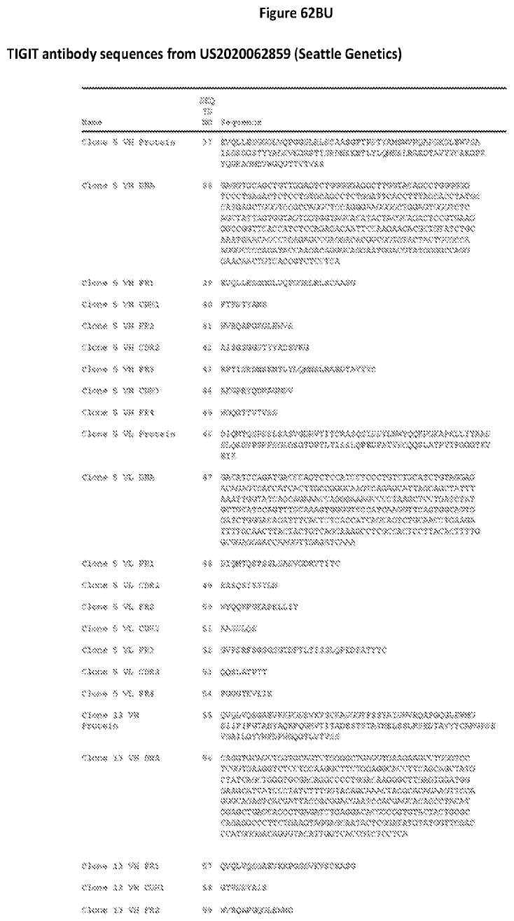

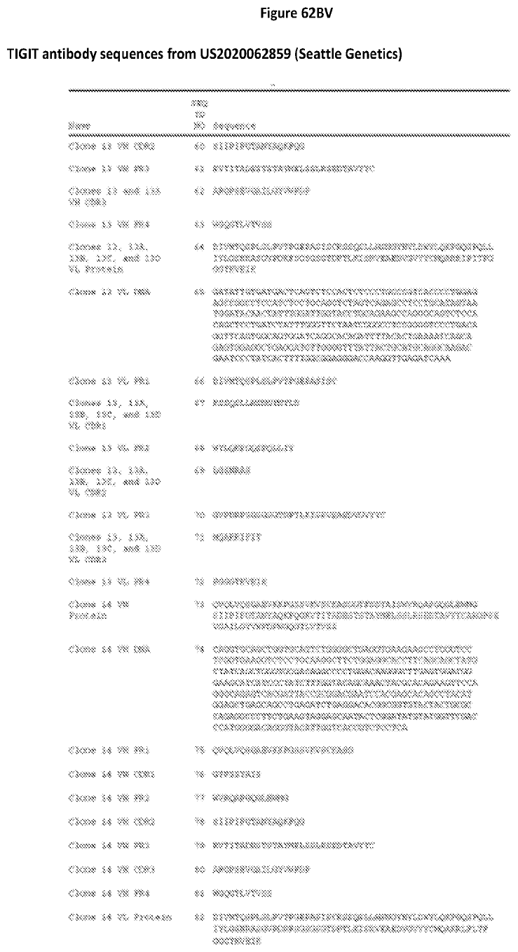

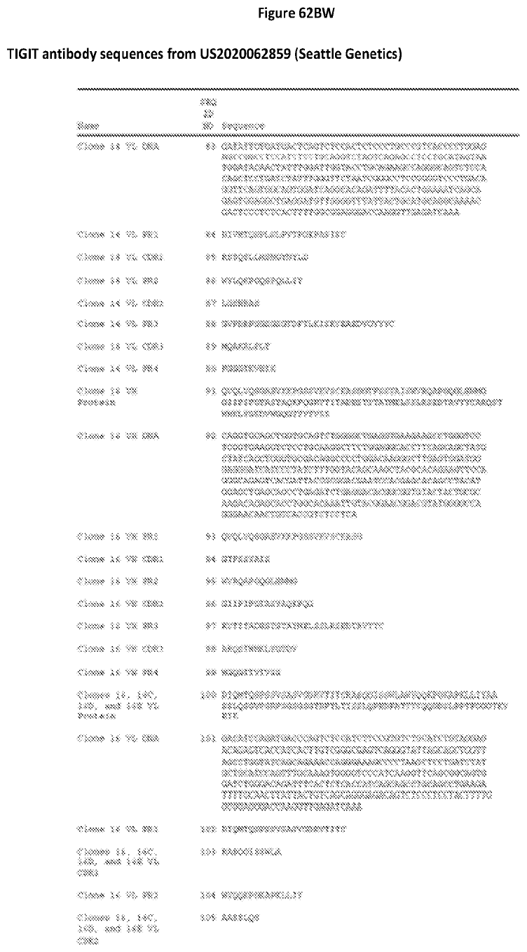

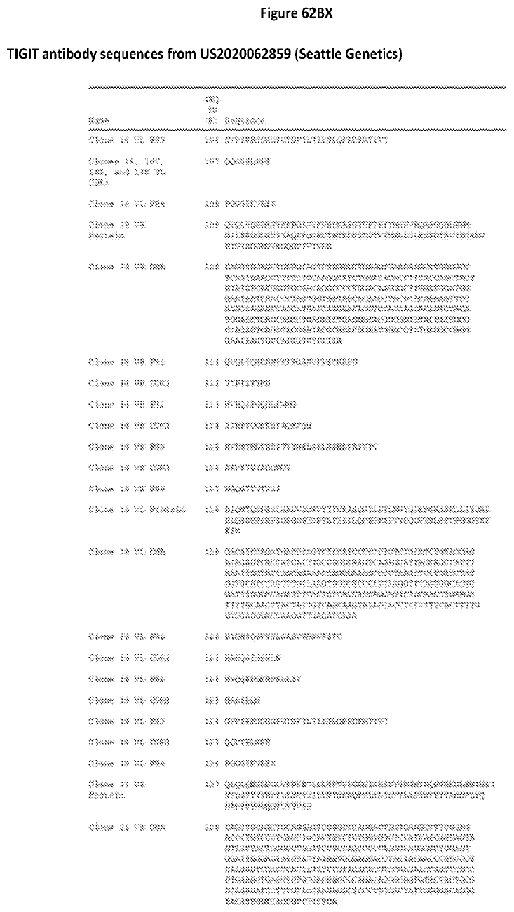

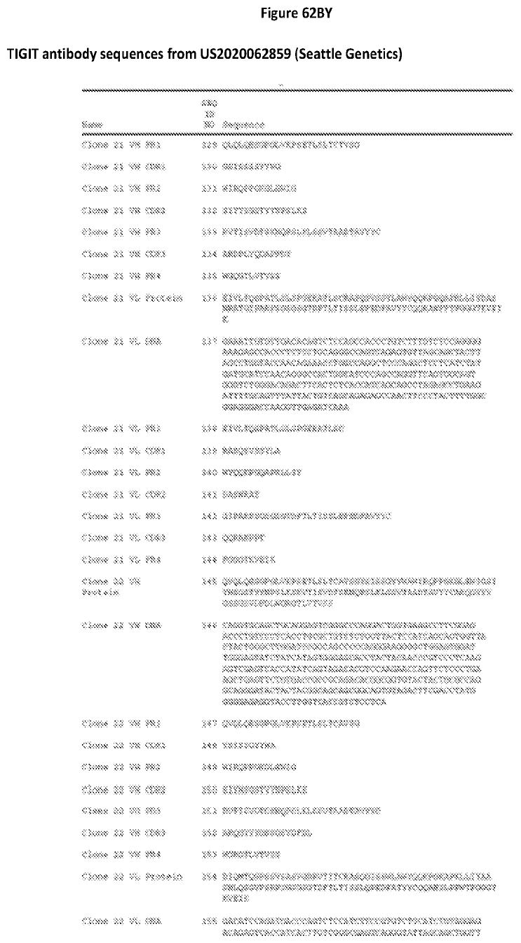

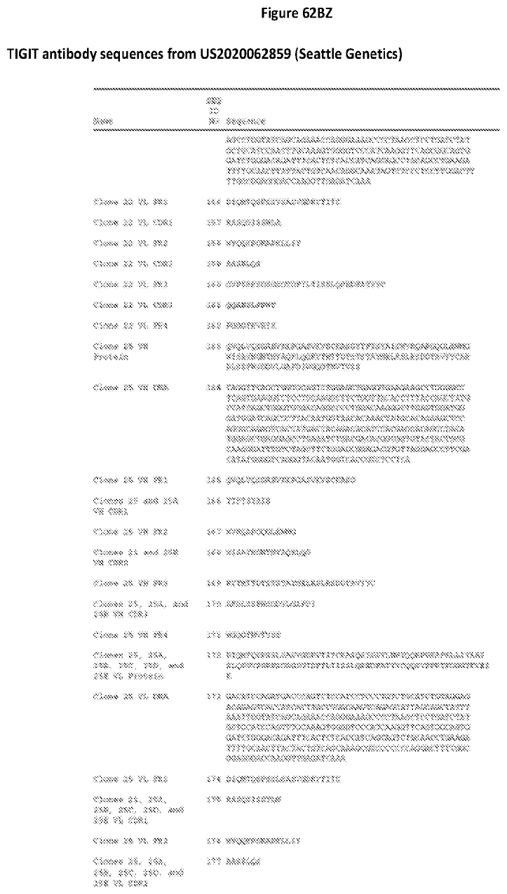

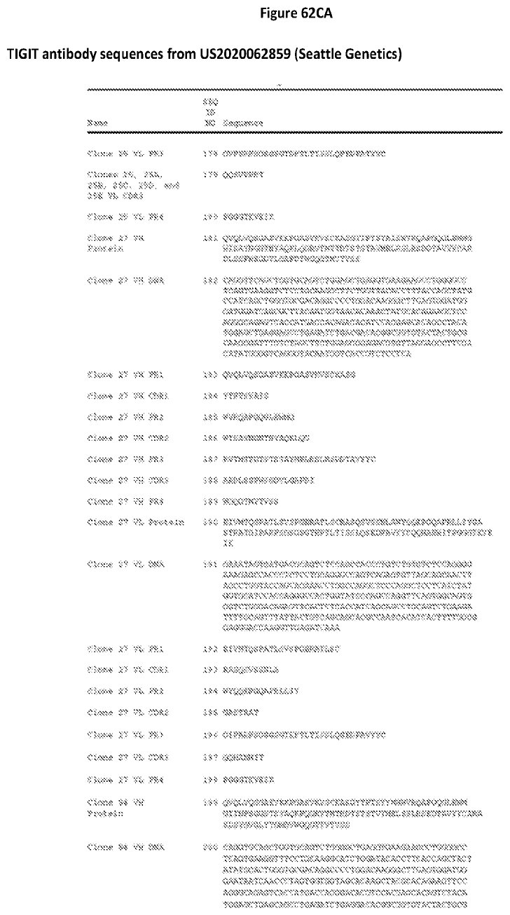

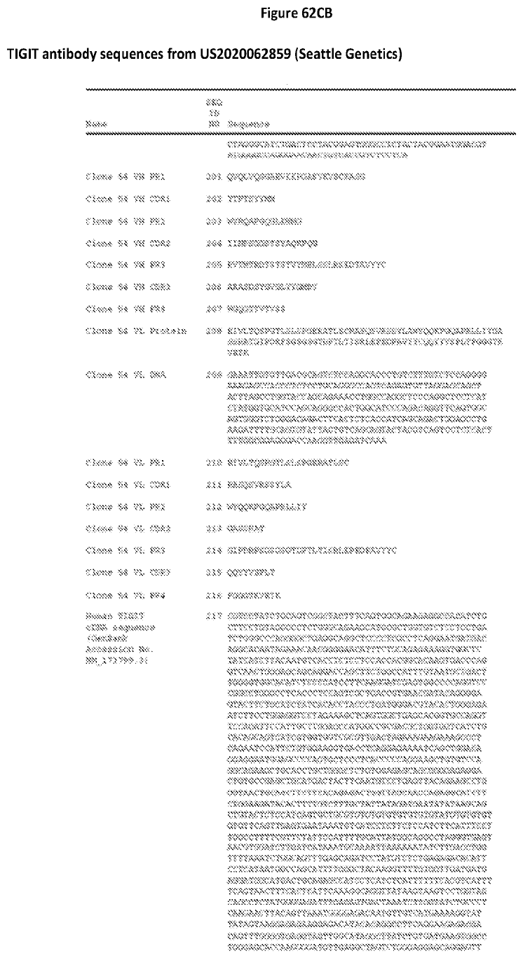

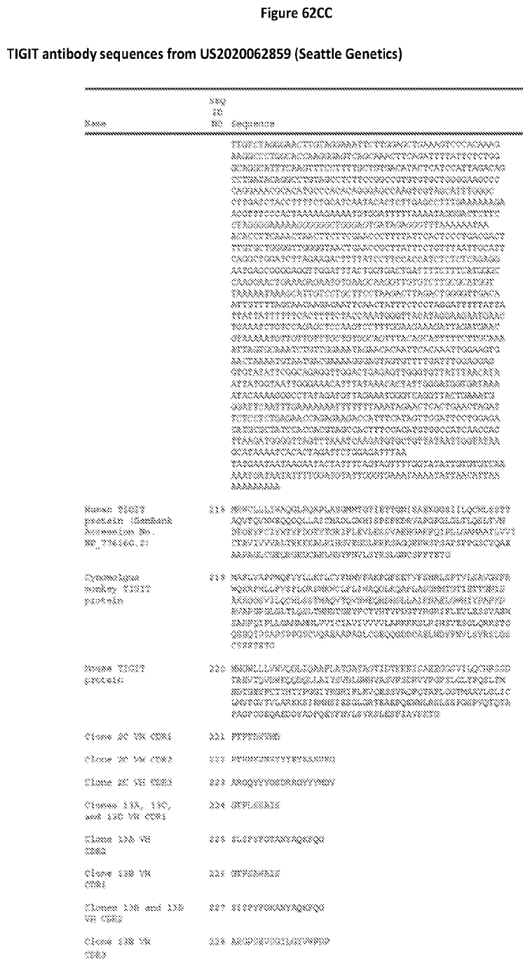

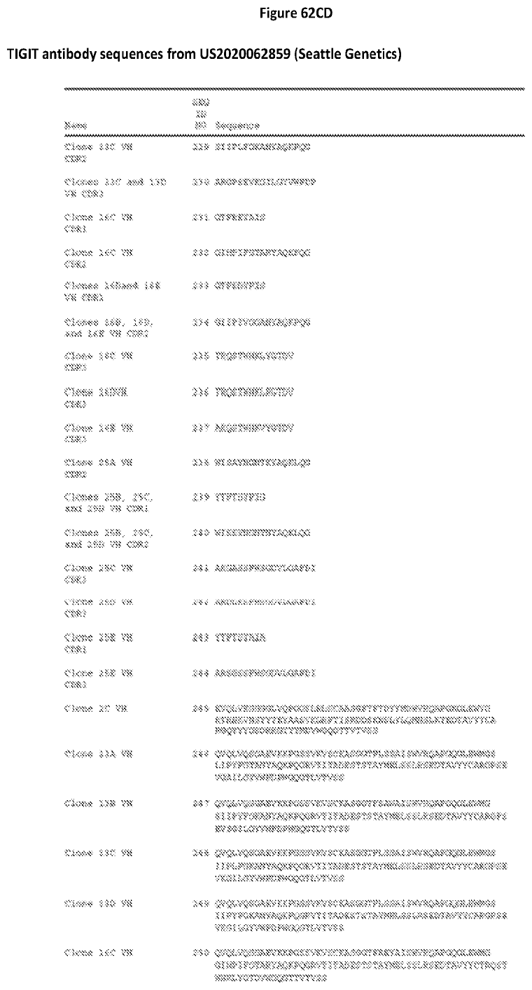

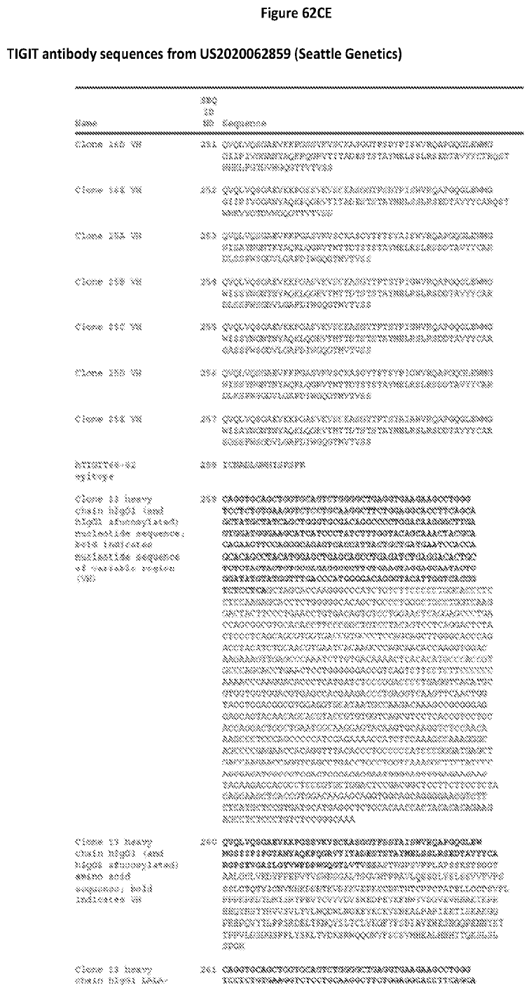

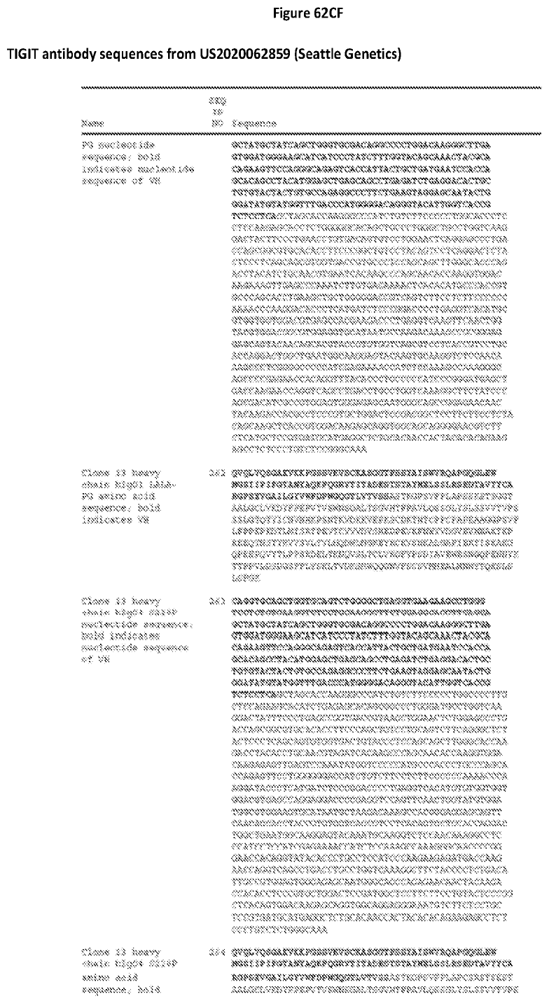

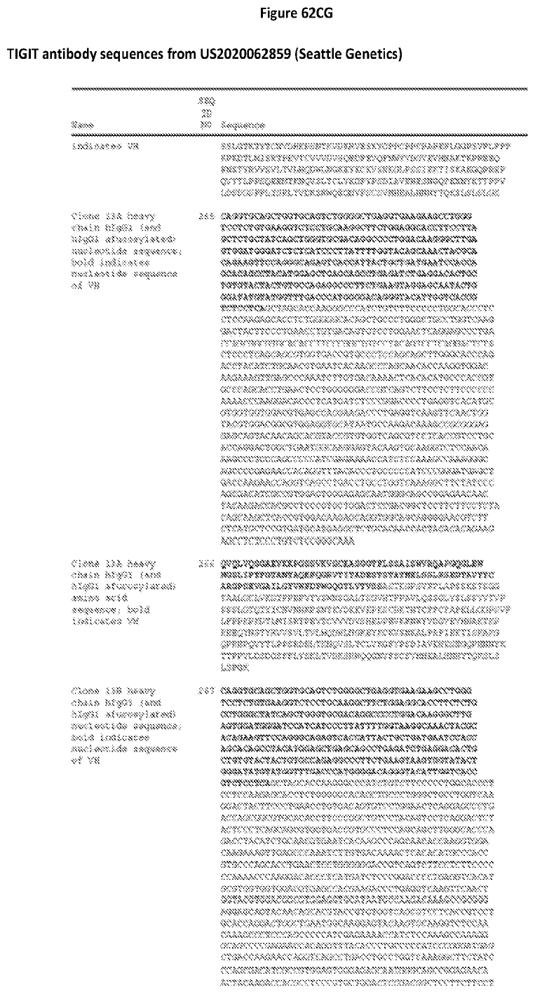

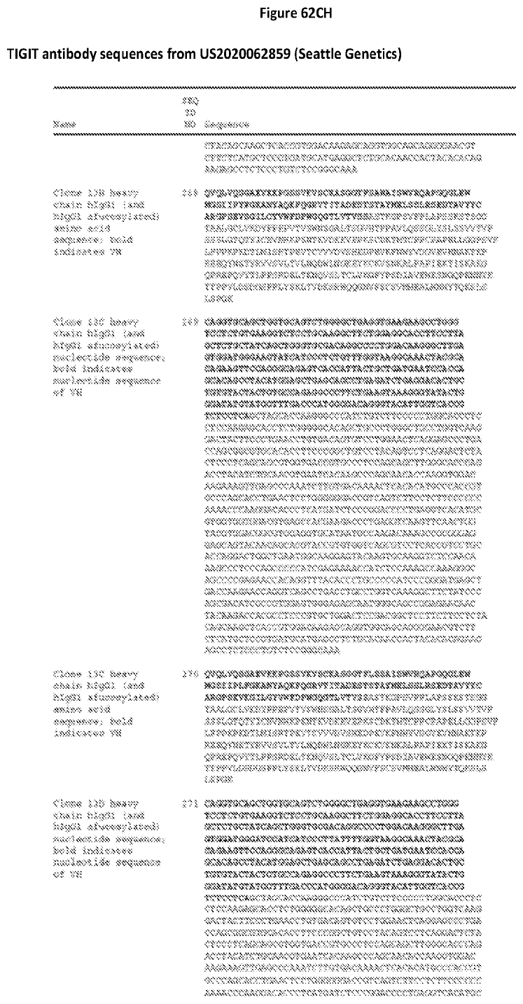

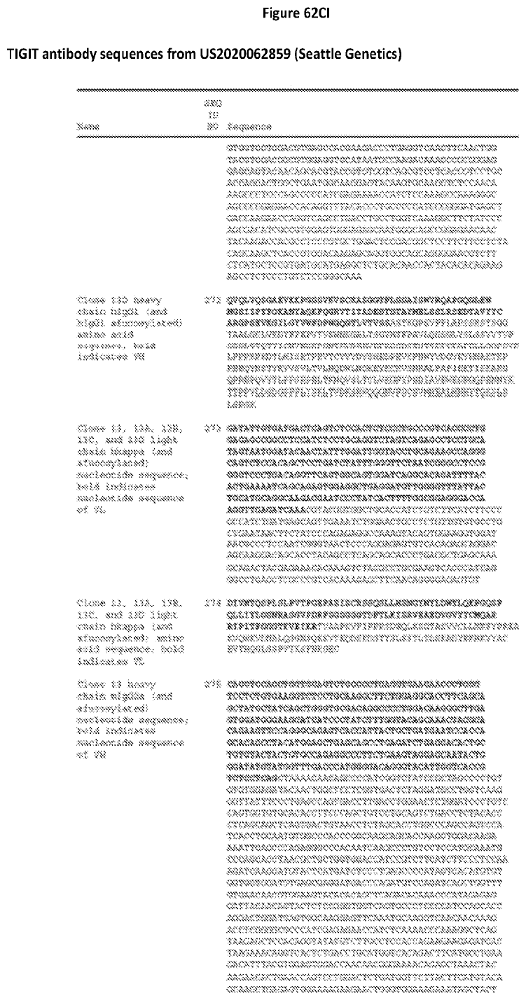

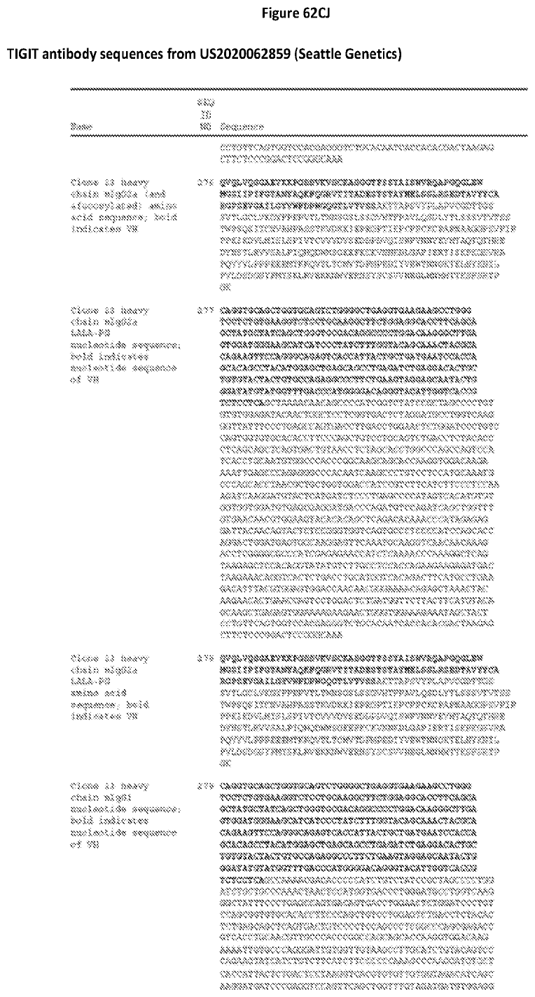

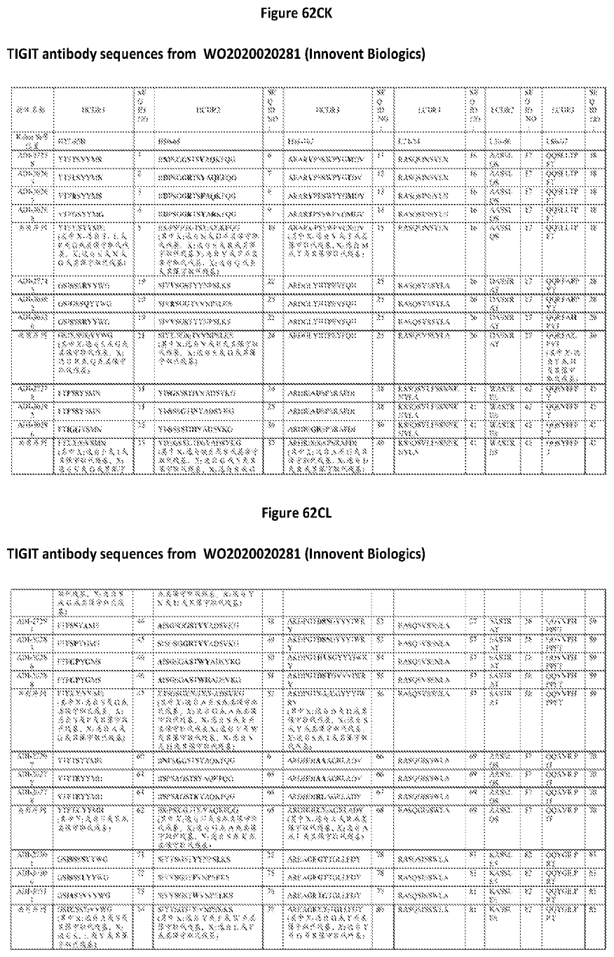

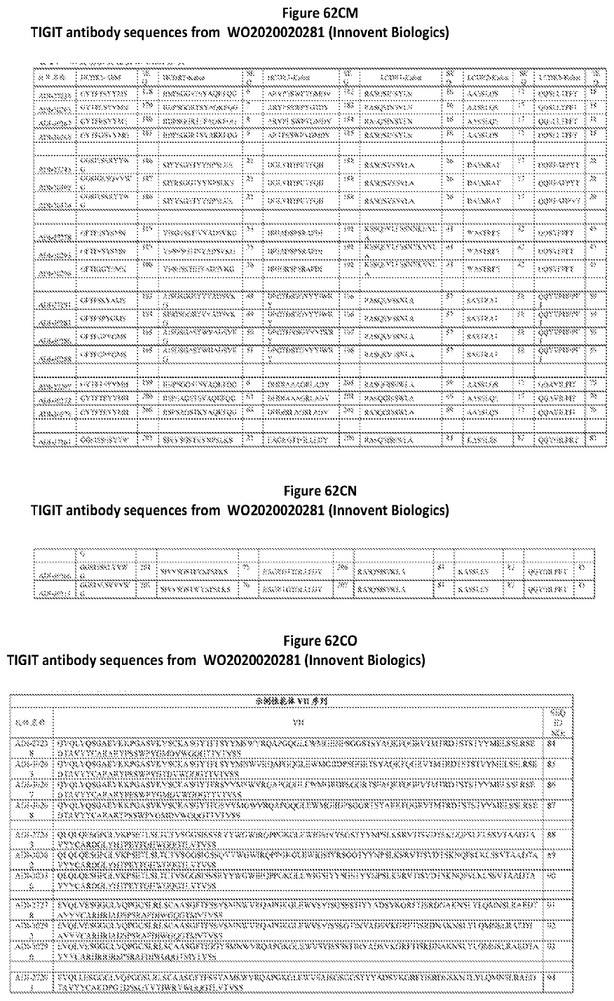

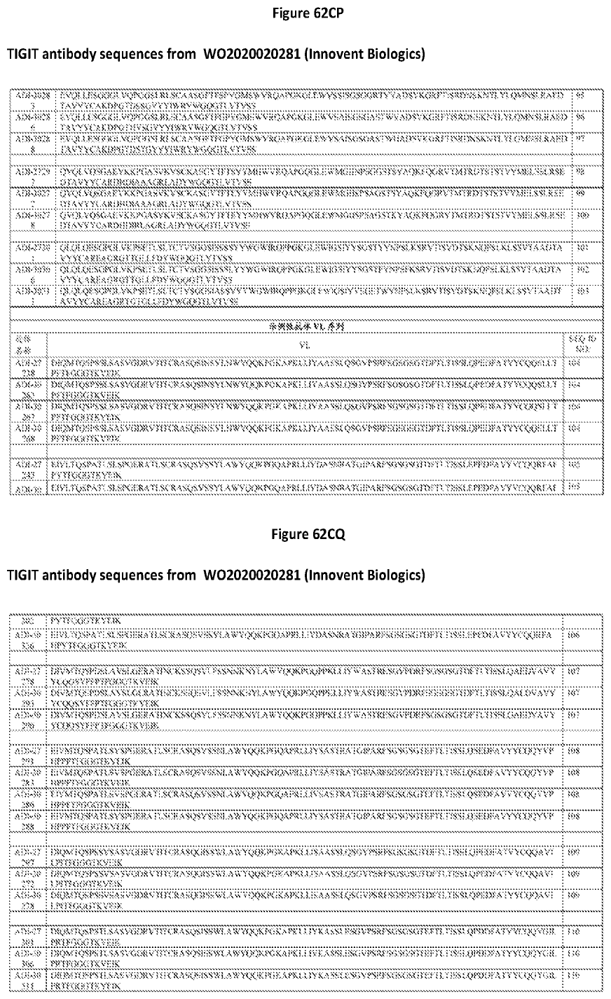

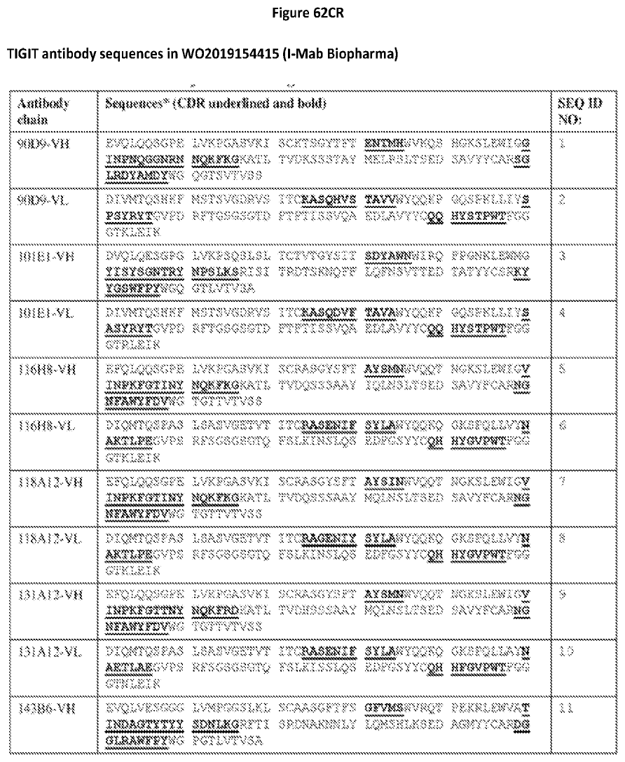

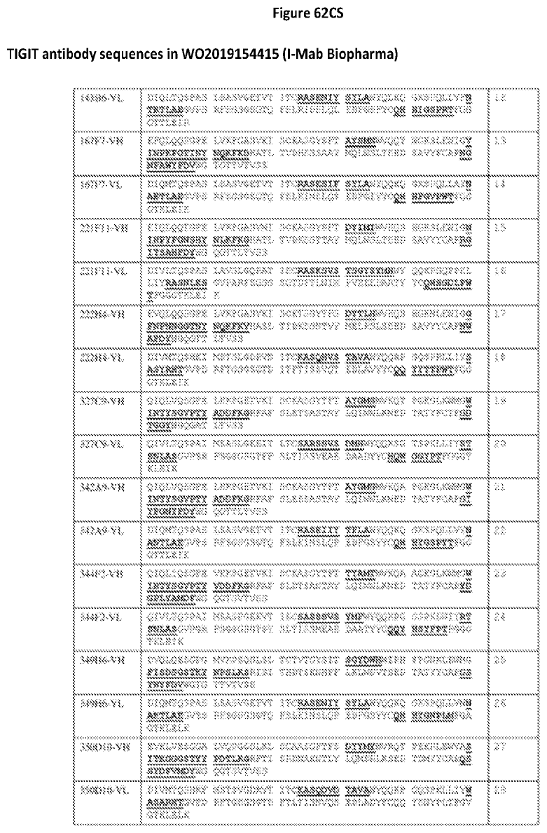

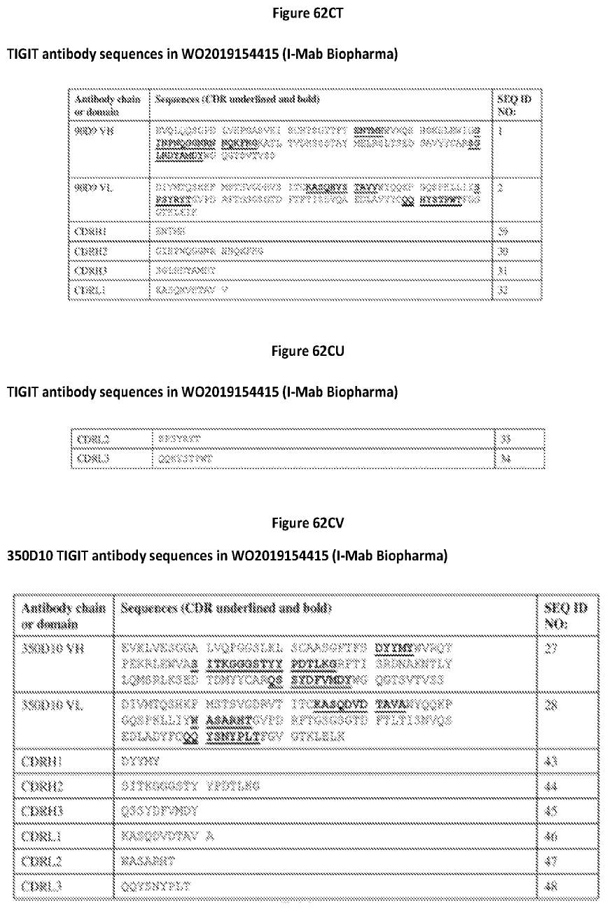

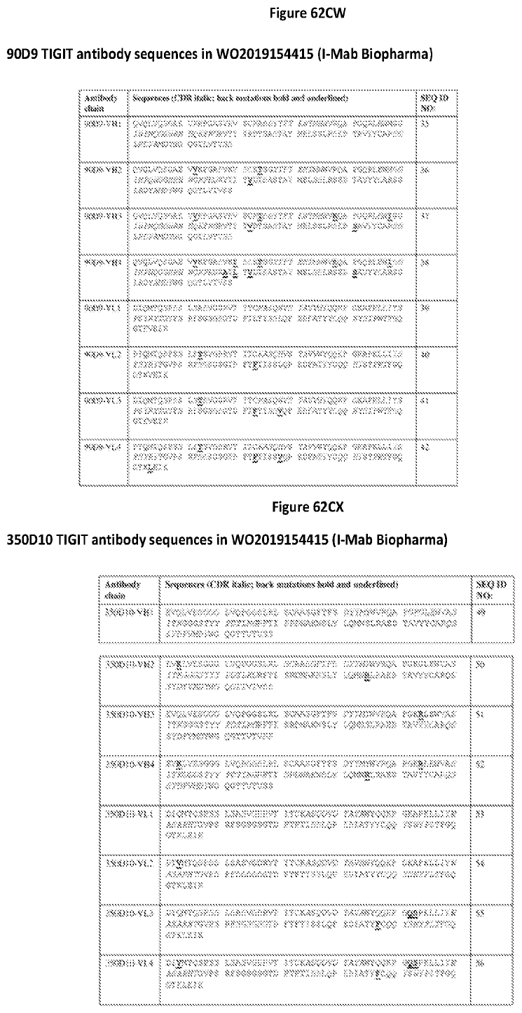

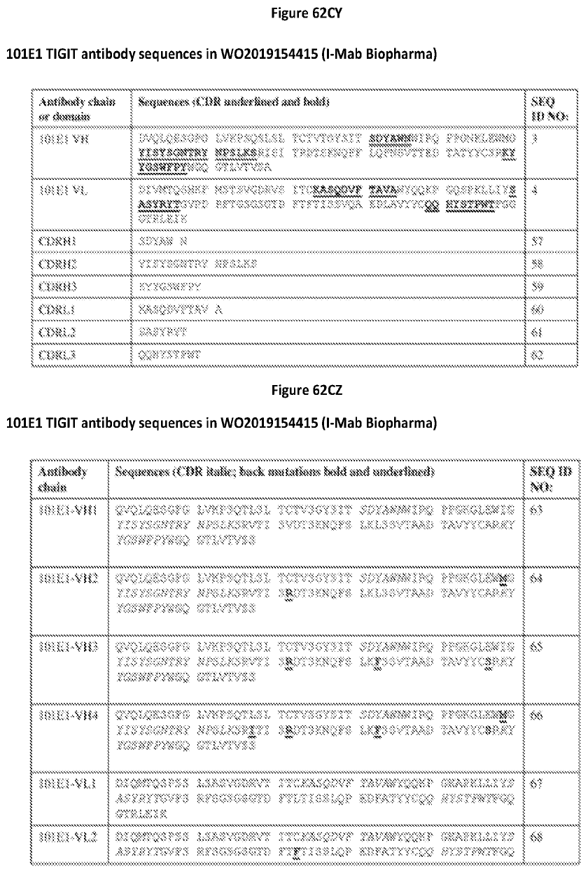

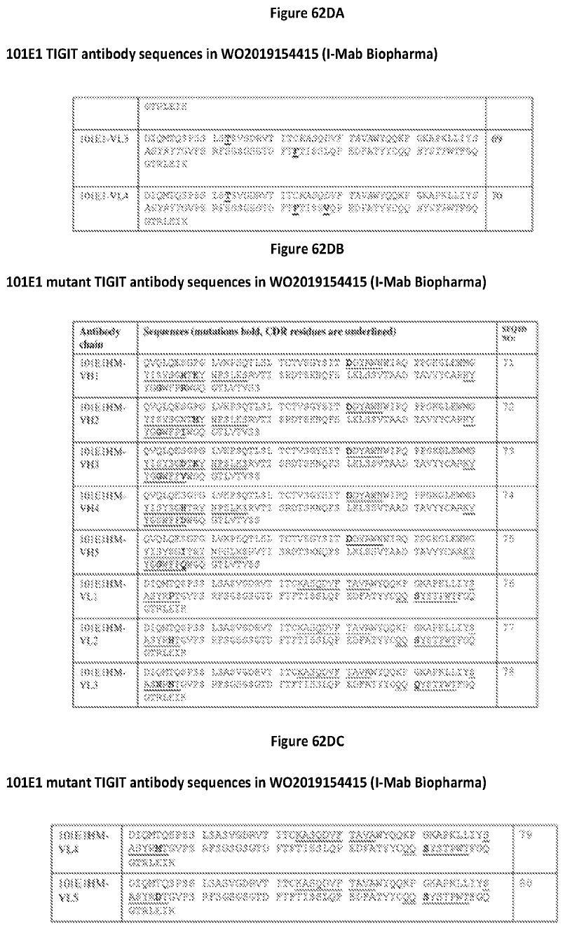

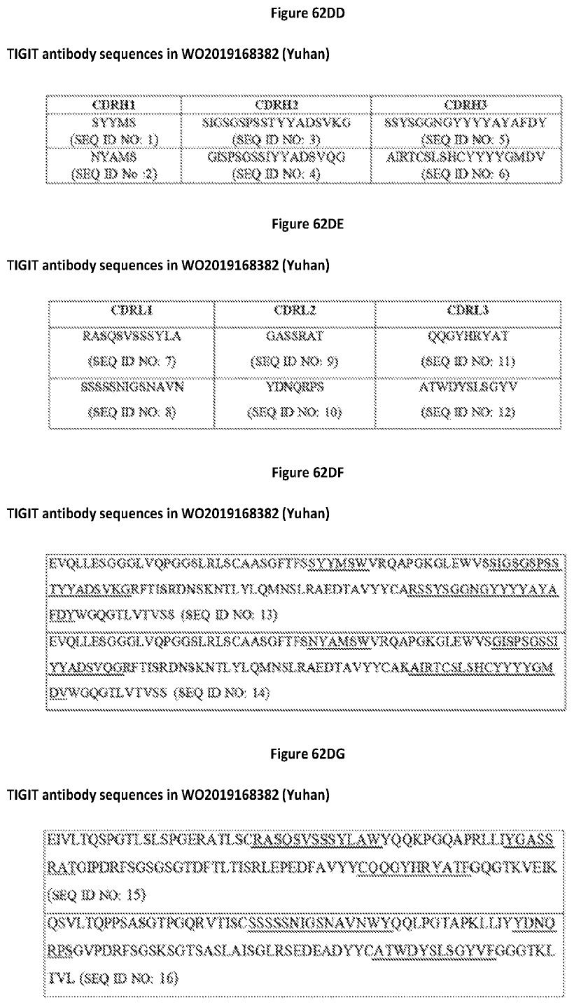

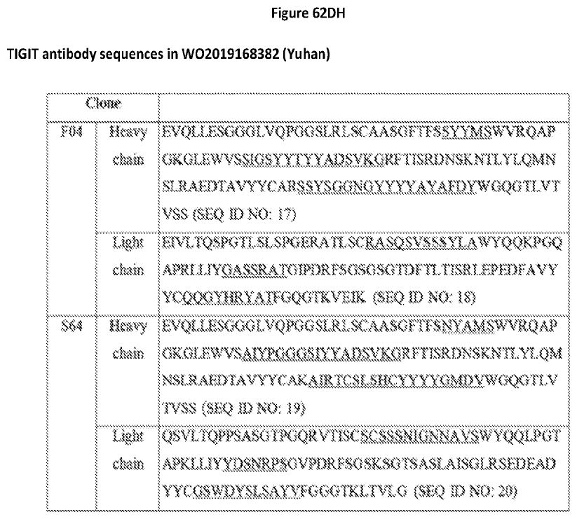

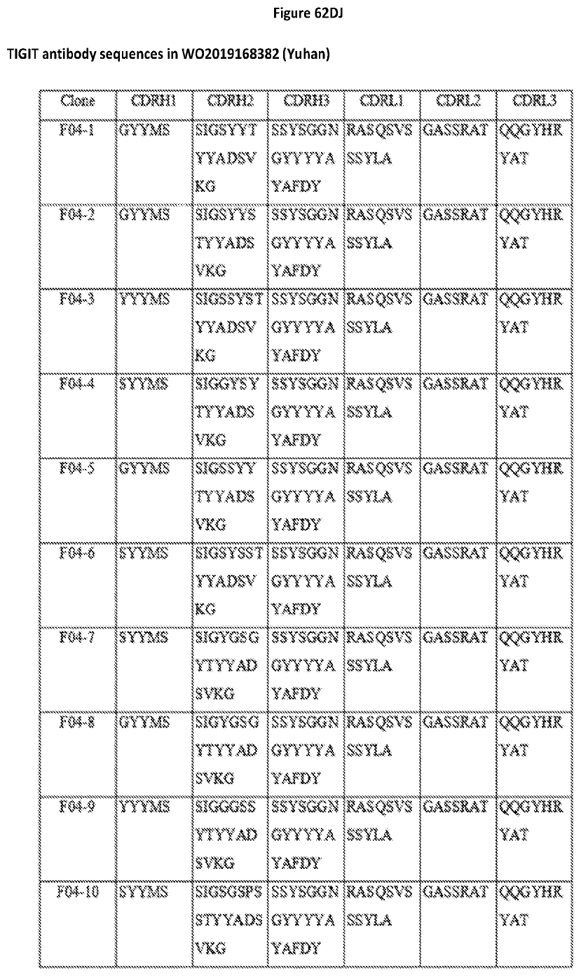

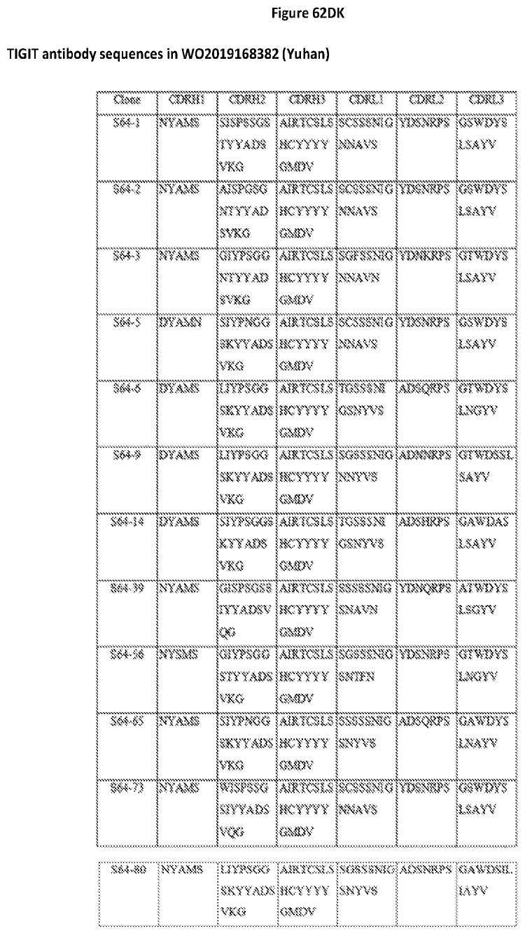

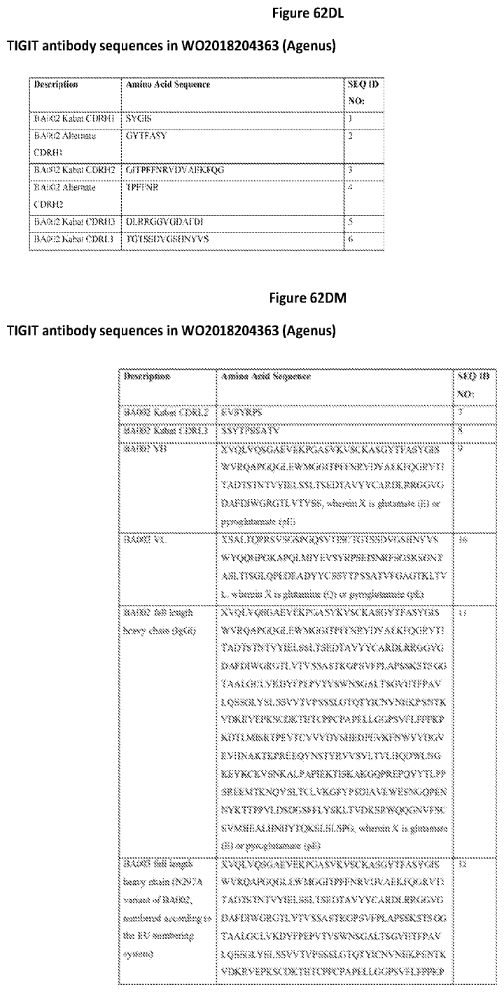

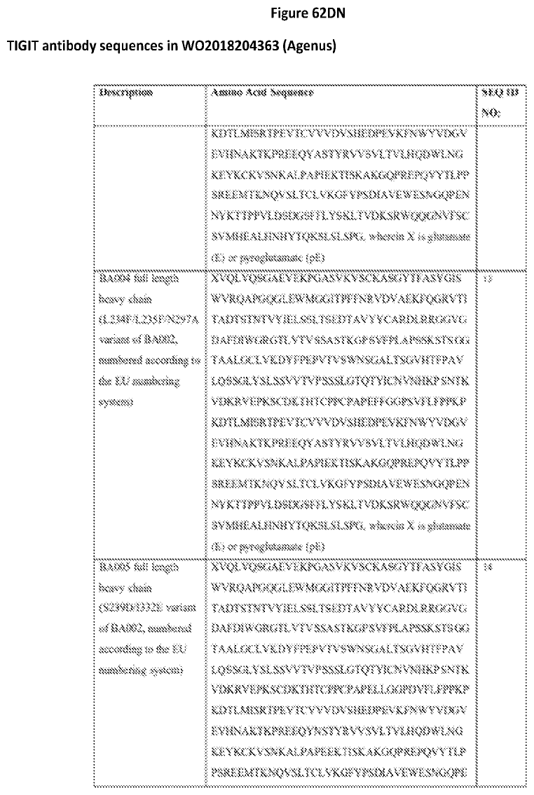

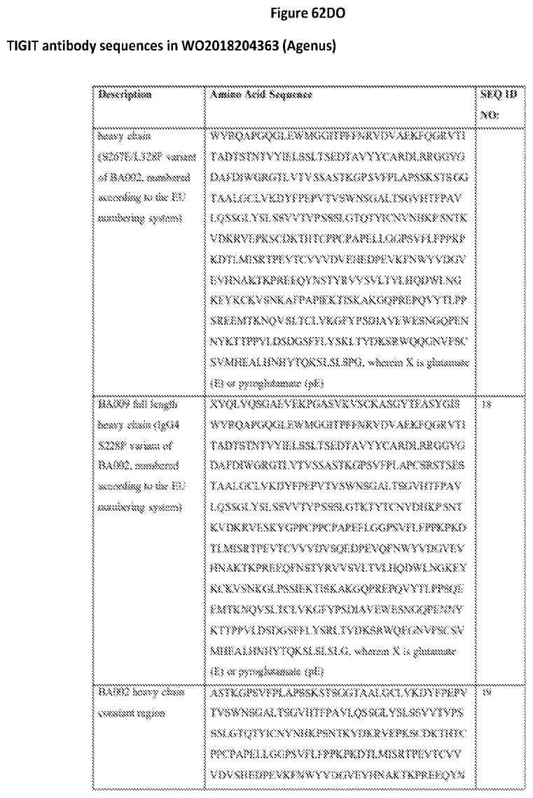

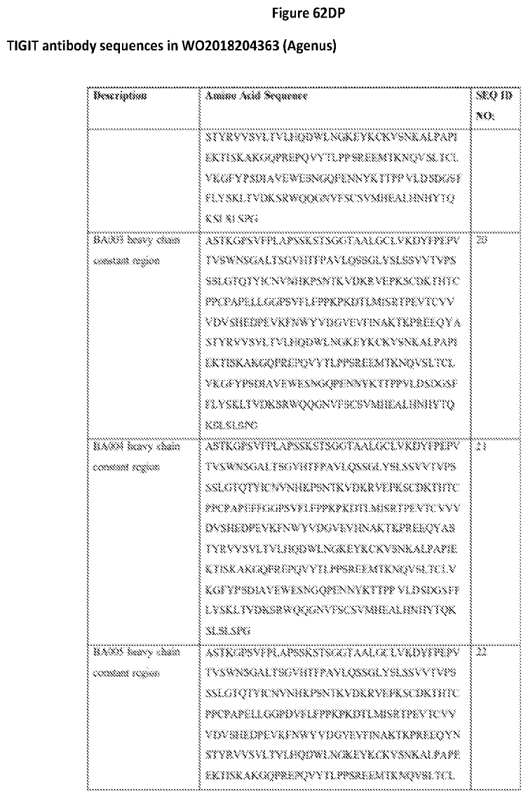

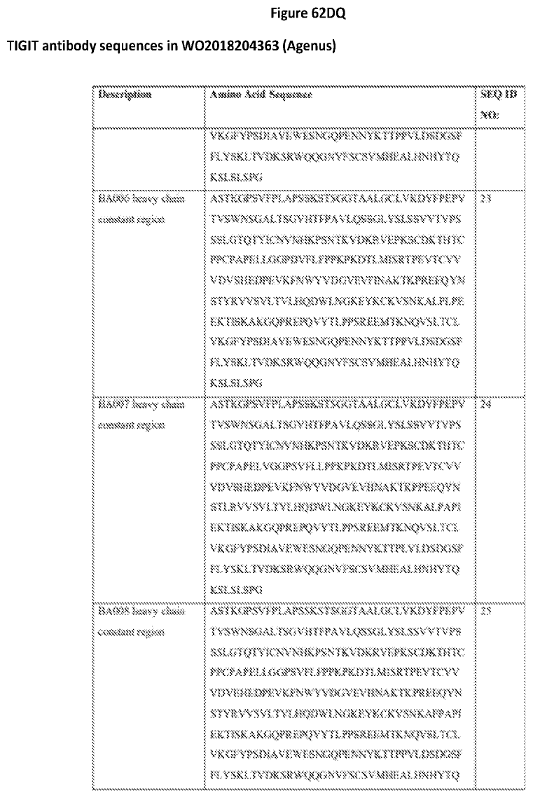

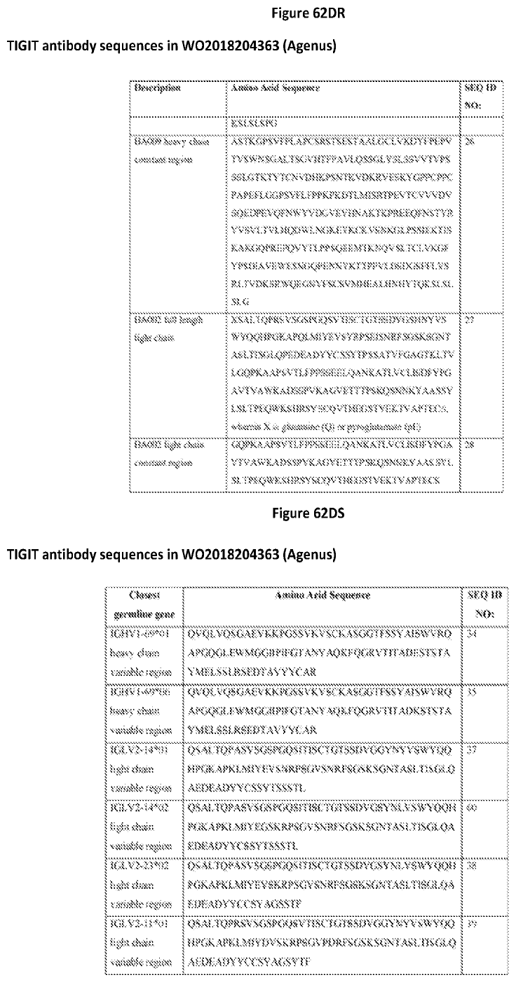

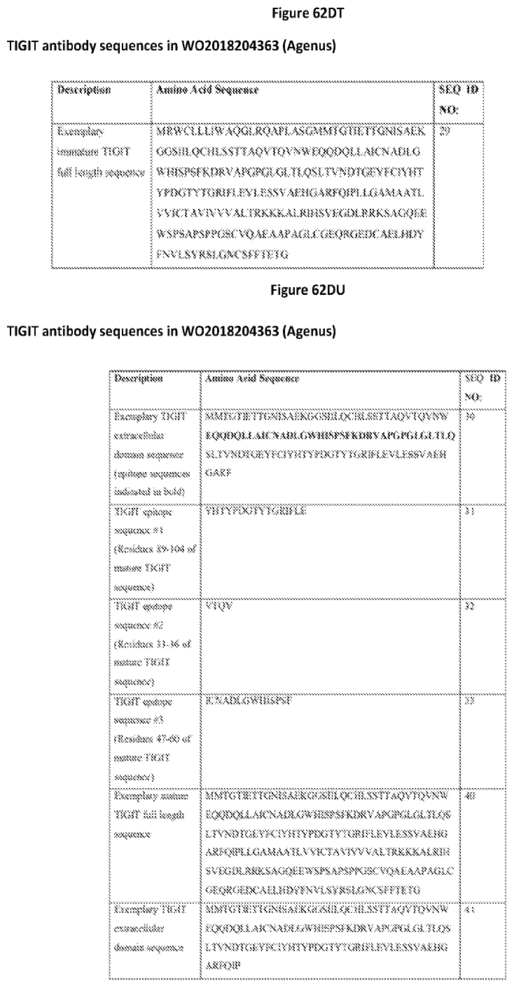

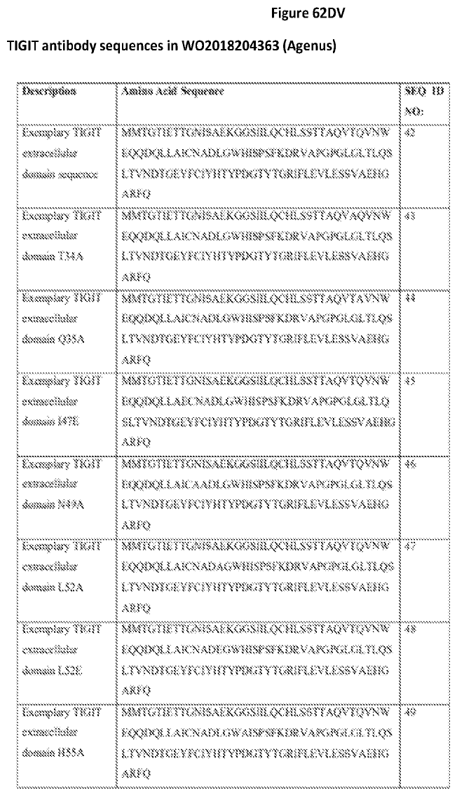

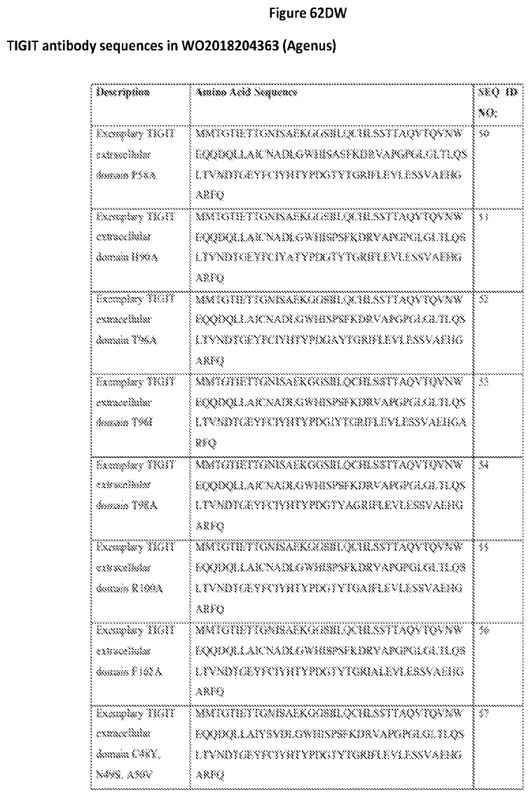

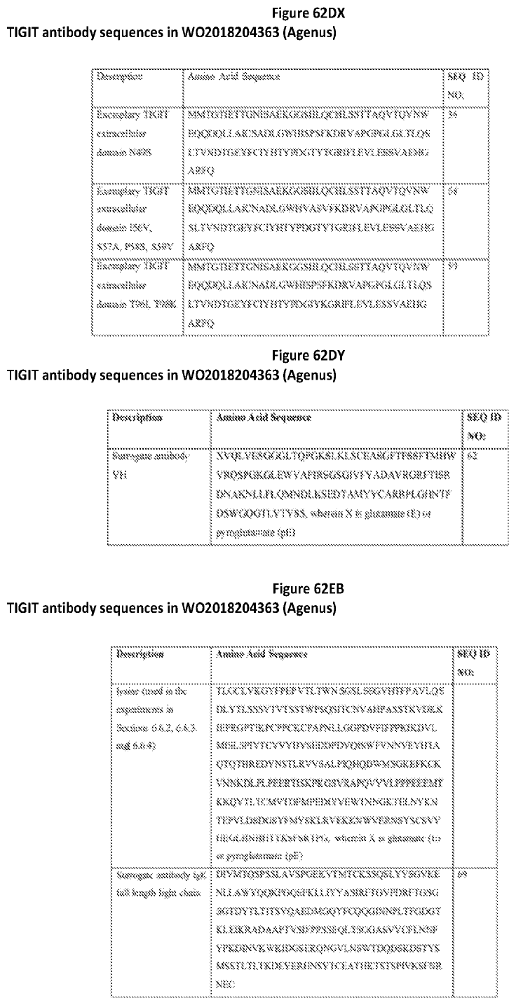

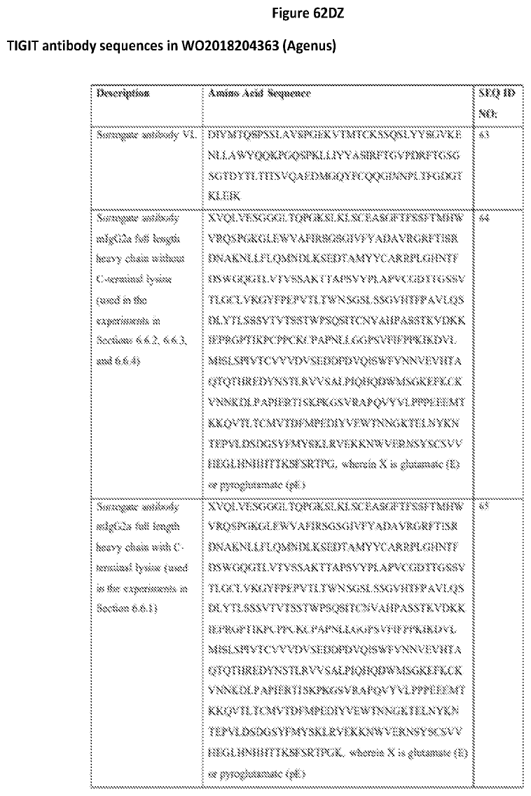

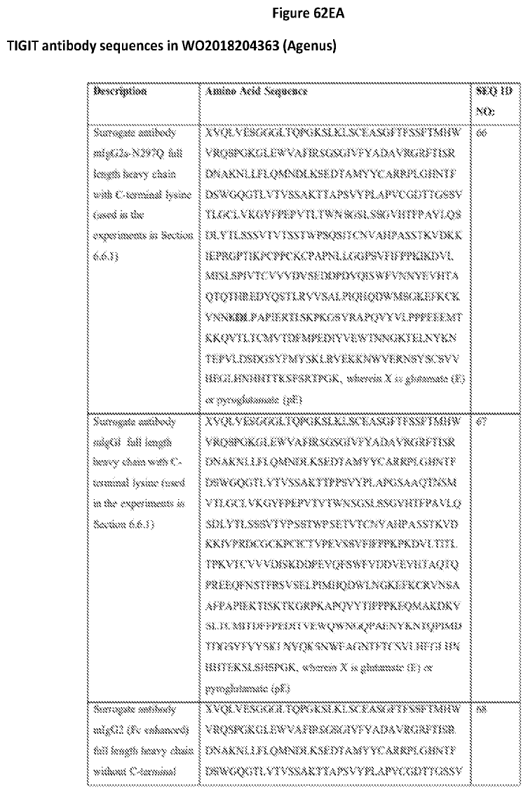

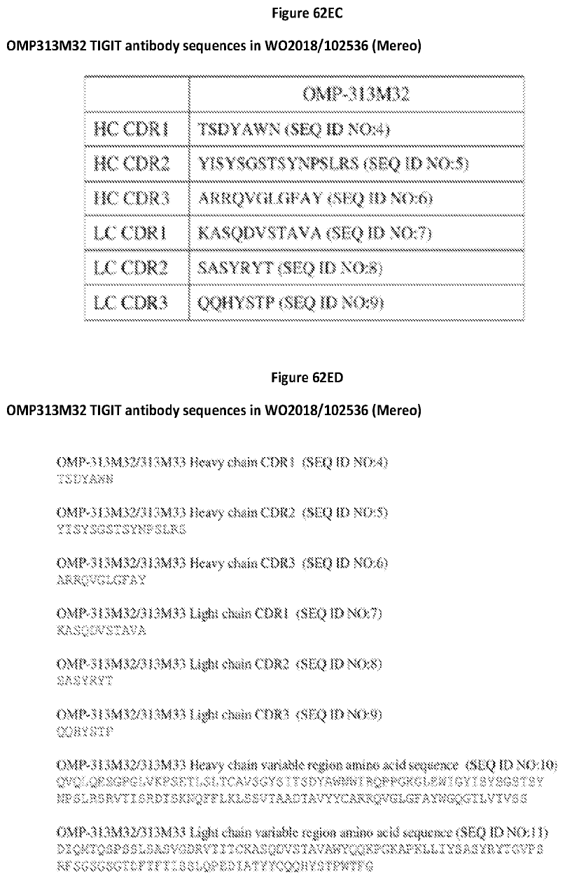

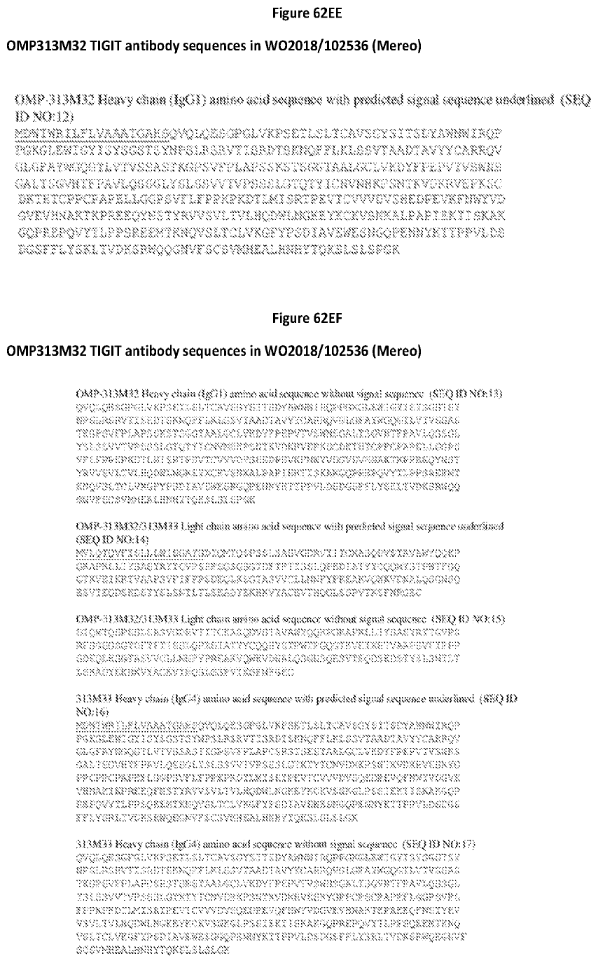

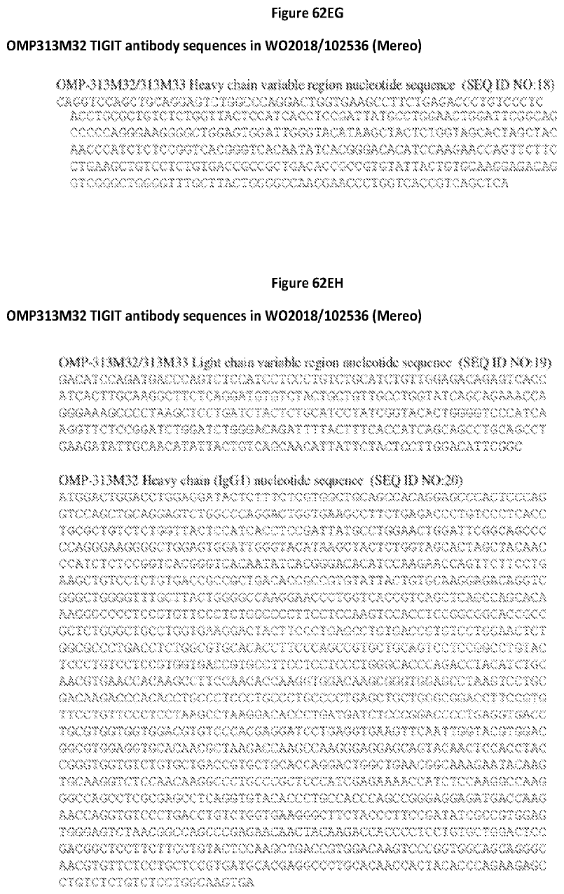

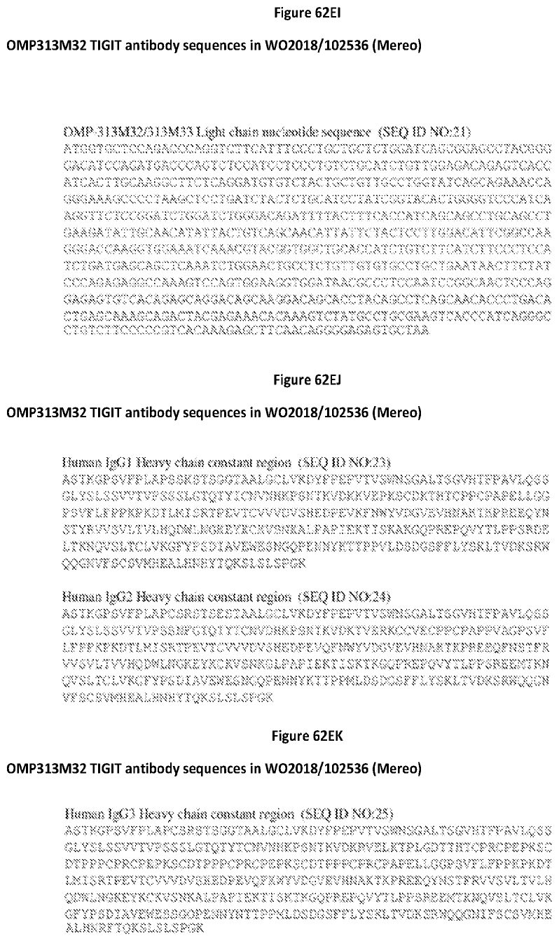

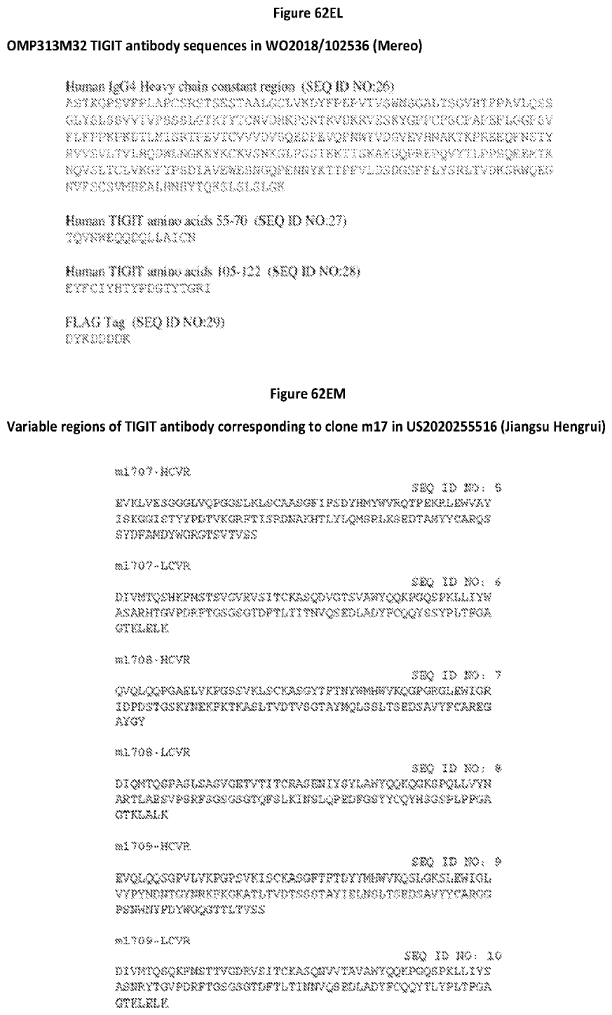

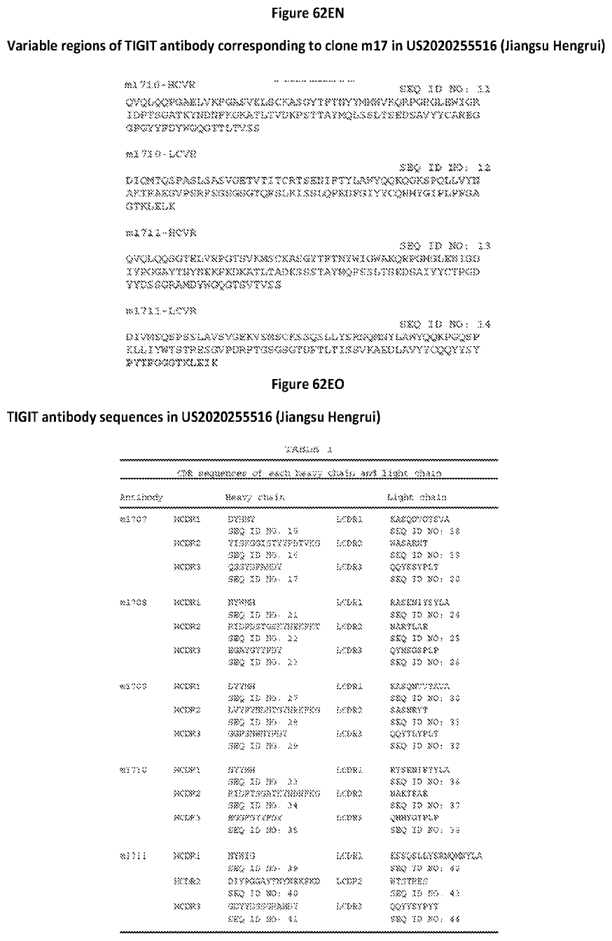

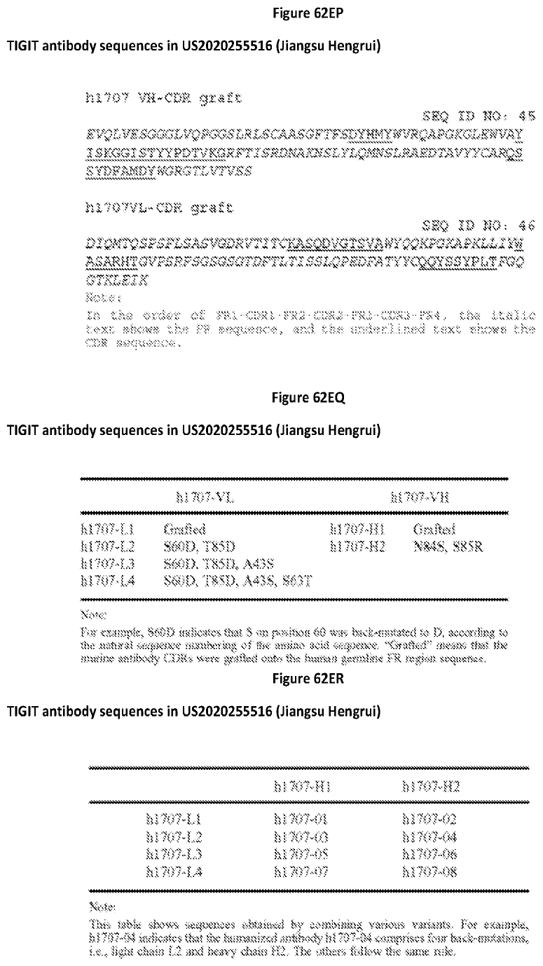

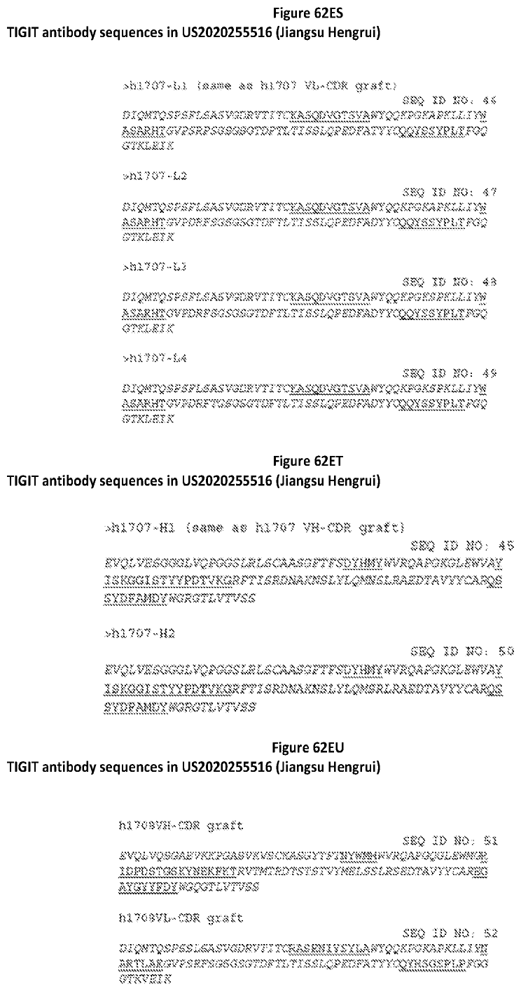

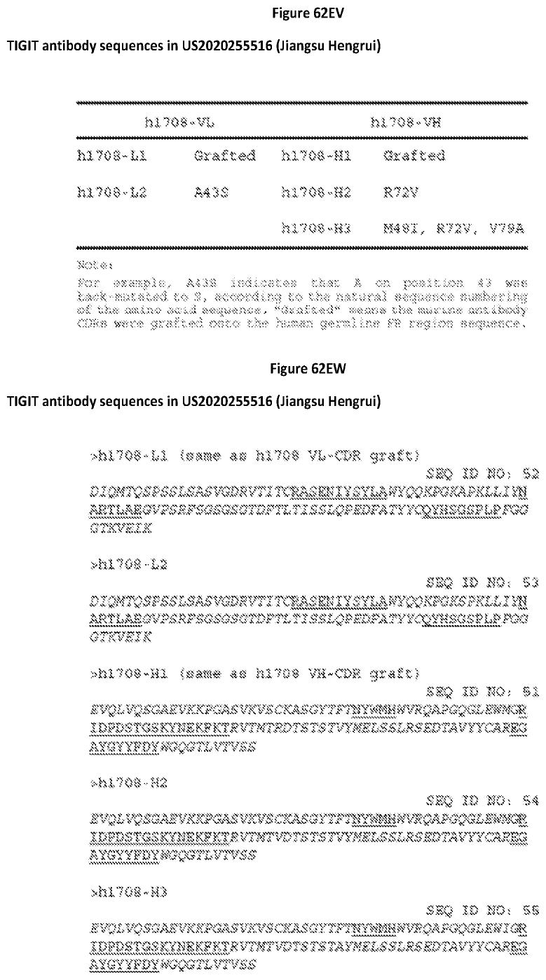

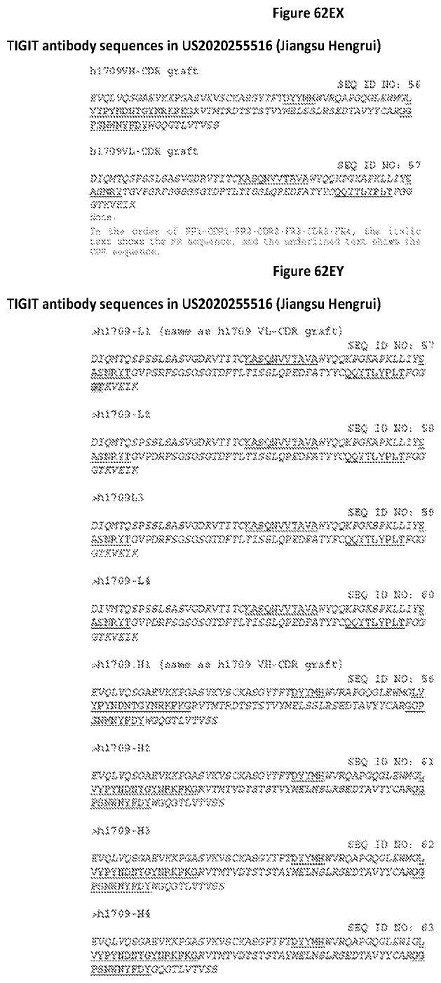

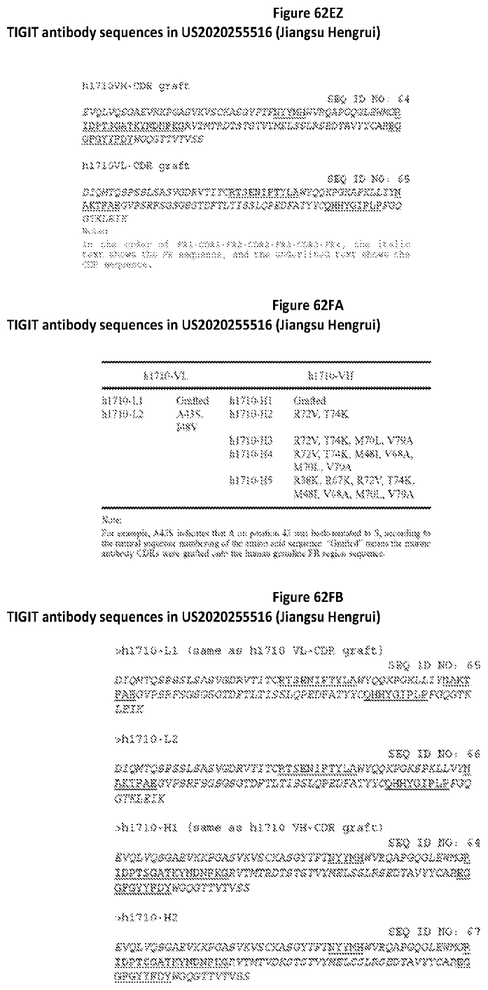

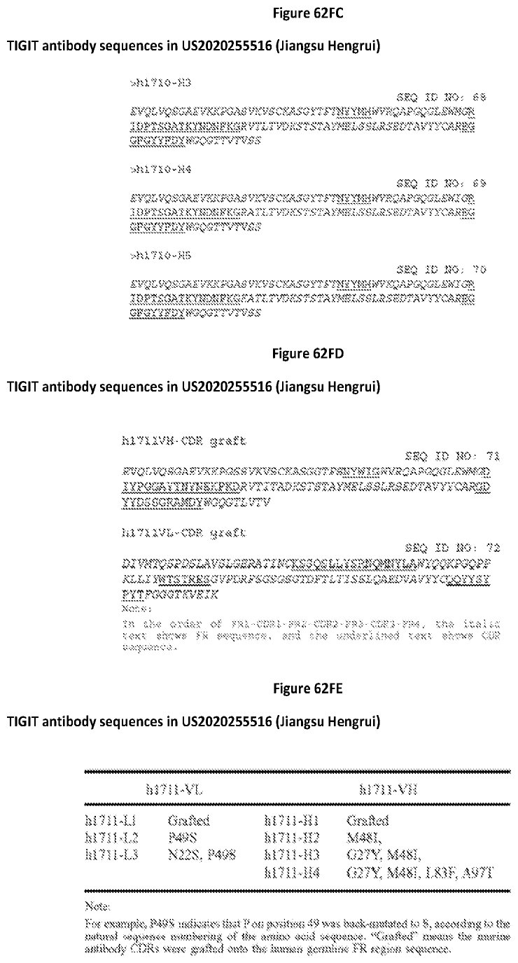

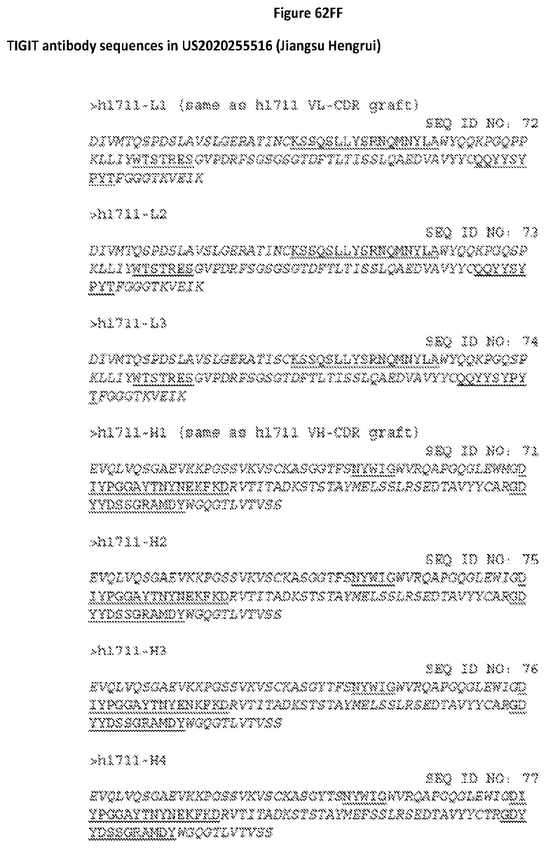

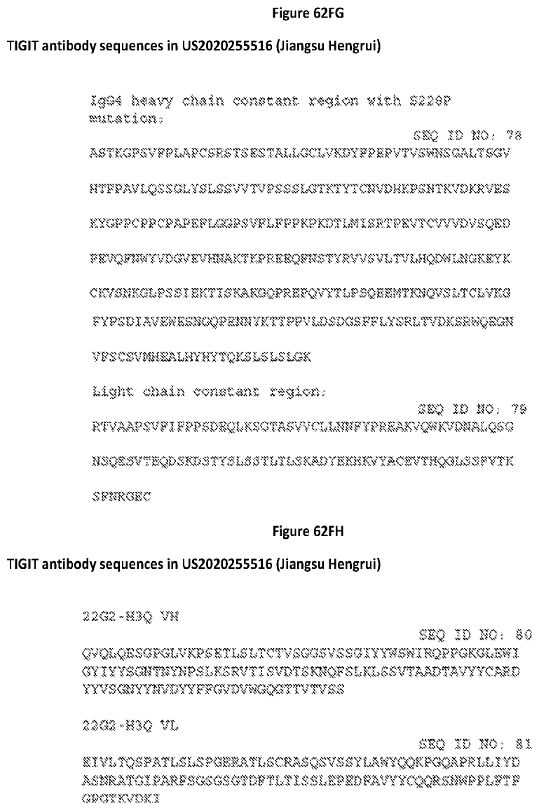

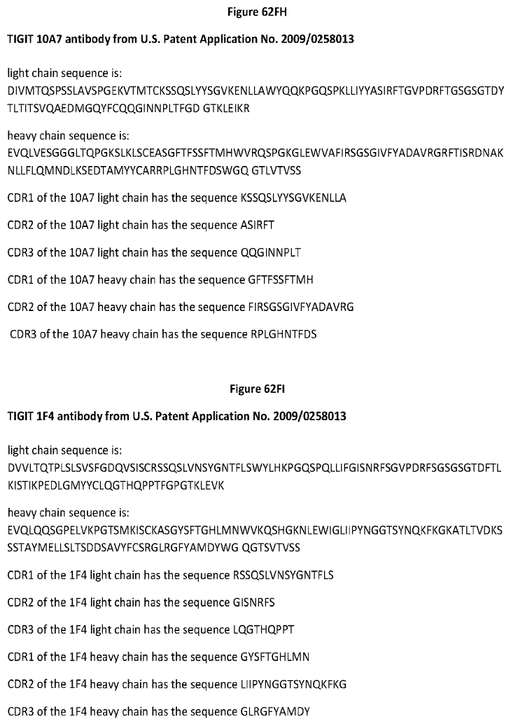

In addition, also included are sequences that may have the identical CDRs but changes in the framework portions of the variable domain (or entire heavy or light chain). For example, TIGIT antibodies include those with CDRs identical to those shown in FIGS. 62A-62FI but whose identity along the variable region can be lower, for example 95 or 98% percent identical.

In some embodiments, the TIGIT binding portion is from an anti-TIGIT antibody as provided in US2016/0176963A1, (incorporated herein by reference in its entirety). In some embodiments, the TIGIT binding portion is from an anti-TIGIT antibody as provided in US20170281764 (incorporated herein by reference in its entirety). In some embodiments, the TIGIT binding portion is from an anti-TIGIT antibody as provided in WO2015009856 (incorporated herein by reference in its entirety). In some embodiments, the TIGIT binding portion is from an anti-TIGIT antibody as provided in U.S. Pat. No. 9,713,641 (incorporated herein by reference in its entirety). In some embodiments, the TIGIT binding portion is from an anti-TIGIT antibody as provided in WO2016028656 (incorporated herein by reference in its entirety).

In some embodiments, the TIGIT binding portion is from an anti-TIGIT antibody as provided in WO2016011264, (incorporated herein by reference in its entirety). In some embodiments, the TIGIT binding portion is from an anti-TIGIT antibody as provided in WO2015009856, (incorporated herein by reference in its entirety). In some embodiments, the TIGIT binding portion is from an anti-TIGIT antibody as provided in US20170281764 (incorporated herein by reference in its entirety). In some embodiments, the TIGIT binding portion is from an anti-TIGIT antibody as provided in WO2016028656 (incorporated herein by reference in its entirety). In some embodiments, the TIGIT binding portion is from an anti-TIGIT antibody as provided in US2016/0176963 (incorporated herein by reference in its entirety). In some embodiments, the TIGIT binding portion is from an anti-TIGIT antibody as provided in U.S. Pat. No. 9,713,641 (incorporated herein by reference in its entirety). In some embodiments, the TIGIT binding portion is from an anti-TIGIT antibody as provided in US2019315867 (incorporated herein by reference in its entirety). In some embodiments, the TIGIT binding portion is from an anti-TIGIT antibody as provided in US2020331999 (incorporated herein by reference in its entirety). In some embodiments, the TIGIT binding portion is from an anti-TIGIT antibody as provided in US2020062859 (incorporated herein by reference in its entirety). In some embodiments, the TIGIT binding portion is from an anti-TIGIT antibody as provided in WO2020020281 (incorporated herein by reference in its entirety). In some embodiments, the TIGIT binding portion is from an anti-TIGIT antibody as provided in WO2019154415 (incorporated herein by reference in its entirety). In some embodiments, the TIGIT binding portion is from an anti-TIGIT antibody as provided in WO2019168382 (incorporated herein by reference in its entirety). In some embodiments, the TIGIT binding portion is from an anti-TIGIT antibody as provided in WO2018204363 (incorporated herein by reference in its entirety). In some embodiments, the TIGIT binding portion is from an anti-TIGIT antibody as provided in CN110818795 (incorporated herein by reference in its entirety). In some embodiments, the TIGIT binding portion is from an anti-TIGIT antibody as provided in US2020255516 (incorporated herein by reference in its entirety).

In some embodiments, the anti-TIGIT antibody of the present invention is TIG1 (JN Biosciences), TIG2 (JN Biosciences), TIG3 (JN Biosciences), MK7684/Vibostolimab (Merck), BMS-986207 (BMS), ASP8374 (Astellas/Potenza), EOS-448 (iTeos Therapeutics), SGN-TGT (Seattle Genetics), IBI-939 (Innovent Biologics), TJT6 (I-Mab Biopharma), 90D9 (I-Mab Biopharma), 350D10 (I-Mab Biopharma), 101E1 (I-Mab Biopharma), YH29143 (Yuhan), AGEN1327 (Agenus), YBL-012 (Y Biologics), MG1131 (MOGAM Institute for Biomedical Research), OMP313M32 (Mereo), M6223 (Merck KgAA), JS006 (Junshi Biosciences), BAT6005 (Bio-Thera Solutions), and/or HLX-53 (Henlius Biopharmaceuticals, the contents of each of which are incorporated herein by reference in their entirety.

In one embodiment, the anti-TIGIT antibody is the Arcus Bio antibody, AB154.

In some embodiments, the anti-TIGIT antibody is an antibody described in any of U.S. Patent Application No. 20170037133, International Patent Publication No. WO 2017/048824, a MBSA43 (commercially available from eBioscience), is anti-TIGIT antibody pab2197 or pab2196 (U.S. Patent Application No. 2017/0081409), E05084448, CASC-674 (available from Adimab LLC), all of which are incorporated herein by reference in their entireties.

In some embodiments, the anti-TIGIT antibody is an antibody described in U.S. Pat. No. 9,713,364 (incorporated herein by reference in its entirety). In some embodiments, the anti-TIGIT antibody is PTZ-201 (ASP8374). In some embodiments, the anti-TIGIT antibody is an antibody selected from the group consisting of MAB1, MAB2, MAB3, MAB4, MAB5, MAB6, MAB7, MAB8, MAB9, MAB10, MAB11, MAB12, MAB13, MAB14, MAB15, MAB16, MAB17, MAB18, 40 MAB19, MAB20, or MAB21, as described in U.S. Pat. No. 9,713,364.

In some embodiments, the anti-TIGIT antibody is an antibody described in U.S. Patent Application No. 2009/0258013, the contents of which is incorporated herein by reference in its entirety. In some embodiments, the anti-TIGIT antibody is an antibody described in U.S. Patent Application No, 2016/0176963, the contents of which is incorporated herein by reference in its entirety. In some embodiments, the anti-TIGIT antibody is selected from the group consisting of 10A7, 1F4, 14A6 (Creative Biolabs), 28H5 (Creative Biolabs), 31C6 (Creative Biolabs), 15A6, 22G2, 11G11, and/or 10D7.

1. TIGIT Antibodies that Compete for Binding

The present invention provides not only the enumerated antibodies but additional antibodies that compete with the enumerated antibodies (the CPA numbers enumerated herein that specifically bind to TIGIT) to specifically bind to the TIGIT molecule. The TIGIT antibodies for use with the invention “bin” into different epitope bins. Among the 44 TIGIT antibodies in the epitope binning study, there are four communities, each having related pairwise blocking patterns, which separate into 12 total discrete bins outlined herein. There are twelve discrete bins outlined herein; 1) BM9-H4, CHA.9.525, CPA.9.081-H4, CHA.9.538, CHA.9.553, CPA.9.069-H4, CHA.9.543, CHA.9.556, CPA.9.077-H4 and CHA.9.561; 2) CHA.9.560 and CHA.9.528; 3) CHA.9.552, CHA.9.521, CHA.9.541, CHA.9.529, CHA.9.519, CHA.9.527 and CHA.9.549;4) CPA.9.057-H4 and CHA.9.554; 5) CHA.9.546, CPA.9.012-H4, CHA.9.547, CPA.9.013-H4, CPA.9.018-H4, MBSA43-M1, Sino PVR-Fc(ligand), CHA.9.555, PVR-Fc M2A(ligand), BM29-H4, CPA.9.027-H4, CPA.9.049-H4 and CPA.9.053-H4; 6) CPA.9.064-H4; 7) BM26-H4; 8) CPA.9.059-H4; 9) CHA.9.535 and CPA.9.009-H4; 10) CHA.9.536, CHA.9.522 and CPA.9.015-H4; 11) CPA.9.011-H4 and BM8-H4 and 12) CPA.9.071-H4. As discussed in WO2018/033798, incorporated herein by reference in its entirety.

Thus, the invention provides anti-TIGIT antibodies that compete for binding with antibodies that are in discrete epitope bins 1 to 12. In a particular embodiment, the invention provides anti-TIGIT antibodies that compete for binding with CPA.9.086 and are at least 95%, 96%, 97%, 98%, or 99% identical to CPA.9.086.

Additional antibodies anti-TIGIT antibodies that compete with the enumerated antibodies are generated, as is known in the art and generally outlined below. Competitive binding studies can be done as is known in the art, generally using SPR/Biacore® binding assays, as well as ELISA and cell-based assays.

### VII. NUCLEIC ACIDS ENCODING ANTIBODIES

Nucleic acid compositions encoding the anti-TIGIT antibodies for use with the invention are also provided, as well as expression vectors containing the nucleic acids and host cells transformed with the nucleic acid and/or expression vector compositions. As will be appreciated by those in the art, the protein sequences depicted herein can be encoded by any number of possible nucleic acid sequences, due to the degeneracy of the genetic code.

The nucleic acid compositions that encode the anti-TIGIT antibodies will depend on the format of the antibody. For traditional, tetrameric antibodies containing two heavy chains and two light chains are encoded by two different nucleic acids, one encoding the heavy chain and one encoding the light chain. These can be put into a single expression vector or two expression vectors, as is known in the art, transformed into host cells, where they are expressed to form the antibodies for use with the invention. In some embodiments, for example when scFv constructs are used, a single nucleic acid encoding the variable heavy chain-linker-variable light chain is generally used, which can be inserted into an expression vector for transformation into host cells. The nucleic acids can be put into expression vectors that contain the appropriate transcriptional and translational control sequences, including, but not limited to, signal and secretion sequences, regulatory sequences, promoters, origins of replication, selection genes, etc.

Preferred mammalian host cells for expressing the recombinant the anti-TIGIT antibodies according to at least some embodiments of the invention include Chinese Hamster Ovary (CHO cells), PER.C6, HEK293 and others as is known in the art.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art.

To create a scFv gene, the V_{H}- and V_{L}-encoding DNA fragments are operatively linked to another fragment encoding a flexible linker, e.g., encoding the amino acid sequence (Gly4-Ser)₃ and others discussed herein, such that the V_{H} and V_{L} sequences can be expressed as a contiguous single-chain protein, with the V_{L} and V_{H} regions joined by the flexible linker.

### VIII. FORMULATIONS

The therapeutic compositions used in the practice of the foregoing methods (and in particular antibodies comprising at least the CDRs from CHA.7.518.1, CHA.7.518.4, CPA.9.086, and/or CHA.9.547.18) can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980). Acceptable carriers, excipients, or stabilizers are nontoxic to recipients at the dosages and concentrations employed and may include buffers.

In a preferred embodiment, the pharmaceutical composition that comprises the the anti-PVRIG and/or anti-TIGIT antibodies for use with the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids and the like. “Pharmaceutically acceptable base addition salts” include those derived from inorganic bases and the like.

Administration of the pharmaceutical composition comprising the anti-PVRIG and/or anti-TIGIT antibodies of the present invention, preferably in the form of a sterile aqueous solution, may be done in a variety of ways, including, but not limited to subcutaneously and intravenously.

The dosing amounts and frequencies of administration are, in a preferred embodiment, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

In order to treat a patient, a therapeutically effective dose of the Fc variant of the present invention may be administered. By “therapeutically effective dose” herein is meant a dose that produces the effects for which it is administered. The exact dose will depend on the purpose of the treatment, and will be ascertainable by one skilled in the art using known techniques.

### IX. METHODS FOR USING ANTIBODIES

The anti-PVRIG and/or anti-TIGIT antibodies for use with the invention antibodies for use with the invention as described herein can be used in a number of diagnostic and therapeutic applications. In some cases, the decision of which the anti-PVRIG and/or anti-TIGIT antibodies to administer to a patient is done using an evaluation of the expression levels (either gene expression levels or protein expression levels, with the latter being preferred) of sample tumor biopsies to determine whether the sample is overexpressing TIGIT and/or PVRIG, to determine what therapeutic antibody to administer. In some embodiments, the anti-PVRIG and anti-TIGIT antibodies are used to induce NK-cell activation as part of a treatment regimen.

### A. NK-Cell Activation

In some embodiments, the anti-PVRIG and/or anti-TIGIT antibodies as described herein can be employed in a method of activating NK-cells comprising administering an anti-PVRIG and anti-TIGIT antibody, wherein administering the combination of an anti-PVRIG and anti-TIGIT antibody results in increased activation of NK-cells, as compared to a control or standard level of NK-cell activation or as compared to unactivated NK-cells level.

In some embodiments, the anti-PVRIG and/or anti-TIGIT antibodies as described herein can be employed in a method of activating NK-cells comprising administering an anti-PVRIG and anti-TIGIT antibody, wherein administering the combination of an anti-PVRIG and anti-TIGIT antibody results in increased activation of NK-cells, optionally as compared to the level of NK-cell activation exhibited for individual administration of an anti-PVRIG or an anti-TIGIT antibody. In some embodiments, the anti-PVRIG and/or anti-TIGIT antibodies as described herein can be employed in a method of activating NK-cells comprising administering an anti-PVRIG and anti-TIGIT antibody, wherein administering the combination of an anti-PVRIG and anti-TIGIT antibody results in increased activation of NK-cells. In some embodiments, the NK-cells activation level is as compared to a control or standard level of NK-cell activation or as compared to unactivated NK-cells level.

In some embodiments, the NK-cell activation finds use for the treatment of cancer.

In some embodiments, the anti-PVRIG antibody for inducing NK-cell activation binds a human PVRIG, and the anti-TIGIT antibody for inducing NK-cell activation binds human TIGIT. In some embodiments, the NK-cells activation level is as compared to a control or standard level of NK-cell activation or as compared to unactivated NK-cells level.

In some embodiments, the NK-cell activation when both an anti-PVRIG and anti-TIGIT antibody are administered is one-fold, two-fold, three-fold, four-fold, five-fold, or more as compared to the level of NK-cell activation exhibited for individual administration of an anti-PVRIG or an anti-TIGIT antibody. In some embodiments, the NK-cell activation level is as compared to a control or standard level of NK-cell activation or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG or an anti-TIGIT antibody. In some embodiments, the NK-cell activation level is as compared to a control or standard level of NK-cell activation or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody. In some embodiments, the NK-cells activation level is as compared to a control or standard level of NK-cell activation or as compared to unactivated NK-cells level. In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation as compared to a control or standard level of NK-cell activation or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody, wherein PVRL2 is expressed on the cancer cells of the individual to which the anti-PVRIG and anti-TIGIT antibodies are being administered. In some embodiments, the NK-cells activation level is as compared to a control or standard level of NK-cell activation or as compared to unactivated NK-cells level. In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation as compared to a control or standard level of NK-cell activation or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-TIGIT antibody. In some embodiments, the NK-cells activation level is as compared to a control or standard level of NK-cell activation or as compared to unactivated NK-cells level. In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation as compared to a control or standard level of NK-cell activation or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-TIGIT antibody, wherein PVR is expressed on the cancer cells of the individual to which the anti-PVRIG and anti-TIGIT antibodies are being administered. In some embodiments, the NK-cells activation level is as compared to a control or standard level of NK-cell activation or as compared to unactivated NK-cells level. In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited as compared to a control or standard level of NK-cell activation or as compared to unactivated NK-cells level.

In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody, wherein PVRL2 is expressed on the cancer cells of the individual to which the anti-PVRIG and anti-TIGIT antibodies are being administered. In some embodiments, the NK-cells activation level is as compared to a control or standard level of NK-cell activation or as compared to unactivated NK-cells level. In some embodiments, the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited as compared to a control or standard level of NK-cell activation or as compared to unactivated NK-cells level.

In some embodiments, the NK-cells exhibit increased cytotoxicity when both an anti-PVRIG and anti-TIGIT antibody are administered.

In some embodiments, the NK-cell increased cytotoxicity when both an anti-PVRIG and anti-TIGIT antibody are administered is one-fold, two-fold, three-fold, four-fold, five-fold, or more as compared to the level of NK-cell cytotoxicity exhibited for individual administration of an anti-PVRIG or an anti-TIGIT antibody.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG or an anti-TIGIT antibody.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG antibody.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG antibody, wherein PVRL2 is expressed on the cancer cells of the individual to which the anti-PVRIG and anti-TIGIT antibodies are being administered.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-TIGIT antibody.

In some embodiments, the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-TIGIT antibody, wherein PVR is expressed on the cancer cells of the individual to which the anti-PVRIG and anti-TIGIT antibodies are being administered.

In some embodiments, the NK-cell activation is measured based on an increase in proliferation of at least a subset of NK-cells.

In some embodiments, the NK-cell activation is measured by increase in expression of activation markers. In some embodiments, the activation markers include CD69, CD107a, granzyme, and/or perforin. In some embodiments, the activation markers can be measured using ELISA assays, including immunoassay based ELISA, as well as immunohistochemistry methods.

In some embodiments, the NK-cell activation is measured based on an increase in immunostimulatory activity. In some embodiments, the immunostimulatory activity includes cytoxic activity.

In some embodiments, the NK-cell activation is measured based on an increase in cytokine secretion. In some embodiments, the cytokines include IFNγ and/or TNF. In some embodiments, the cytokines measured is IFNγ. In some embodiments, the cytokines measured is TNF. In some embodiments, the amount of human interferon gamma (IFNγ) in the co-culture supernatant was measured by flow cytometry using a cytometric bead assay (BD). In some embodiments, the amount of IFNγ can be measured using ELISA assays, including immunoassay based ELISA. In some embodiments, the amount of IFNγ can be measured using ELISA assays, including immunoassay based ELISA.

In some embodiments, the NK-cell activation is measured based on an increase in direct killing of target cells by NK-cells in vitro.

In some embodiments, the NK-cell activation is measured based on an increase in direct killing of target cells by NK-cells in vivo.

In some embodiments, the NK-cell activation is measured based on cell surface receptor expression of CD25. In some embodiments, the cell surface receptor expression of CD25 can be measured using ELISA assays, including immunoassay based ELISA, as well as immunohistochemistry methods.

In some embodiments, the anti-PVRIG antibody for inducing NK-cell activation comprises:
a. a heavy chain variable domain comprising a vhCDR1, vhCDR2, and vhCDR3 from an anti-PVRIG antibody; and
b. a light chain variable domain comprising a vlCDR1, vlCDR2, and vlCDR3 from an anti-PVRIG antibody;
wherein the anti-PVRIG antibody in a) and b) is selected from the group consisting of CHA.7.518.4, CHA.7.518.1, CHA.7.518, CHA.7.524 CHA.7.530, CHA.7.538_1, CHA.7.538_2, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549,CHA.7.550, CHA7.538.1.2, CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019,CPA.7.022, CPA.7.023, CPA.7.024, CPA.7.033, CPA.7.034, CPA.7.036, CPA.7.040, CPA.7.046, CPA.7.047, CPA.7.049, CPA.7.050, CHA.7.518, and the antibodies as depicted in FIGS. 24A-24D and 61A-61P.

In some embodiments, the anti-TIGIT antibody for inducing NK-cell activation comprises:
a. a heavy chain variable domain comprising a vhCDR1, vhCDR2, and vhCDR3 from an anti-TIGIT antibody; and
b. a light chain variable domain comprising a vlCDR1, vlCDR2, and vlCDR3 from an anti-TIGIT antibody;
wherein the anti-TIGIT antibody in a) and b) is selected from the group consisting of CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8 and the antibodies as depicted in FIGS. 23A-23EE.

In some embodiments, the PVRIG antibody for inducing NK-cell activation comprises the vlCDR1, vlCDR2, vlCDR3, vhCDR1, vhCDR2, and vhCDR3 from CHA.7.518.1.H4(S241P) and the TIGIT antibody comprises the vlCDR1, vlCDR2, vlCDR3, vhCDR1, vhCDR2, and vhCDR3 from CPA.9.086.H4(S241P).

In some embodiments, the PVRIG antibody is CHA.7.518.1.H4(S241P) and the TIGIT antibody is CPA. 9.086.H4(S241P).

In some embodiments, the anti-PVRIG antibody and/or the anti-TIGIT for inducing NK-cell activation comprises:
a. a heavy chain comprising VH-CH1-hinge-CH2-CH3; and
b. a light chain comprising VL-CL, wherein the CL is the constant domain of either a kappa or lambda antibody.

In some embodiments of the antibody the anti-PVRIG antibody and/or the anti-TIGIT for inducing NK-cell activation, the CL is kappa.

In some embodiments of the antibody the anti-PVRIG antibody and/or the anti-TIGIT for inducing NK-cell activation, the CL is lambda.

In some embodiments, the anti-PVRIG antibody and/or the anti-TIGIT antibody for inducing NK-cell activation is a humanized antibody.

### B. Cancer Treatment

The anti-PVRIG antibodies and/or anti-TIGIT antibodies for use with the invention antibodies for use with the invention find particular use in the treatment of cancer as a monotherapy. Due to the nature of an immuno-oncology mechanism of action, PVRIG and/or TIGIT do not necessarily need to be overexpressed on or correlated with a particular cancer type; that is, the goal is to have the anti-PVRIG antibodies and/or anti-TIGIT antibodies de-suppress T cell and NK cell activation, such that the immune system will go after the cancers. In some embodiments, the anti-PVRIG and anti-TIGIT antibodies are used to induce NK-cell activation as part of a treatment regimen.

In some embodiments, anti-PVRIG antibodies comprise the anti-PVRIG antibody sequences of FIG. 4A-4AA, 5A-5H, 7A-7AE, 11A-11I, 12A-12E, 13, 14A-14BX, 15A-15B, 16A-16E, and 17A-17C, find use in the treatment of cancer (including the activation of T cells and/or NK cells as outlined below), in particular those comprising and S241P substitution. While any anti-PVRIG comprising the anti-PVRIG antibody sequences of FIG. 4A-4AA, 5A-5H, 7A-7AE, 11A-11I, 12A-12E, 13, 14A-14BX, 15A-15B, 16A-16E, and 17A-17C, find use in the treatment of cancer (including the activation of T cells and/or NK cells as outlined below), anti-PVRIG antibodies comprising CHA.7.518.1, CHA.7.518.4, and/or CHA.7.538.2 find particular use in some embodiments.

While any anti-TIGIT antibodies comprising the anti-TIGIT antibody sequences of FIGS. 22A-22D and 23A-23EE find use in the treatment of cancer (including the activation of T cells/and or NK cells as outlined below), anti-TIGIT antibodies comprising CPA.9.086.H4(S241P), CPA.9.083.H4(S241P), CHA.9.547.7.H4(S241P), and CHA.9.547.13.H4(S241P), CHA.9.547.18 find particular use in some embodiments.

In some embodiments, the present invention also provides anti-PVRIG antibodies comprising the anti-PVRIG antibody sequences of FIGS. 4A-4AA, 5A-5H, 7A-7AE, 11A-11I, 12A-12E, 13, 14A-14BX, 15A-15B, 16A-16E, an d17A-17C, which find use in the treatment of cancer (including the activation of T cells/and or NK cells as outlined below).

The anti-PVRIG antibodies and/or anti-TIGIT antibodies for use with the invention find particular use in the treatment of cancer. In general, the anti-PVRIG antibodies and/or anti-TIGIT antibodies for use with the invention are immunomodulatory, in that rather than directly attack cancerous cells, the antibodies for use with the invention stimulate the immune system, generally by inhibiting the action of the checkpoint receptor (e.g., PVRIG or TIGIT). Thus, unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (e.g., antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells.

Accordingly, the anti-PVRIG antibodies and/or anti-TIGIT antibodies for use with the invention are useful in treating cancer. Due to the nature of an immuno-oncology mechanism of action, the checkpoint receptor (TIGIT or PVRIG) does not necessarily need to be overexpressed on or correlated with a particular cancer type; that is, the goal is to have the antibodies de-suppress T cell and NK cell activation, such that the immune system will go after the cancers.

“Cancer,” as used herein, refers broadly to any neoplastic disease (whether invasive or metastatic) characterized by abnormal and uncontrolled cell division causing malignant growth or tumor (e.g., unregulated cell growth.) The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth. Examples of cancer are exemplified in the working examples and also are described within the specification.

Non-limiting examples of cancer that can be treated using the anti-PVRIG antibodies and/or anti-TIGIT antibodies for use with the invention include, but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), esophageal cancer, melanoma, mesothelioma, merkel cell cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, prostate cancer, vulval cancer, thyroid cancer, hepatic carcinoma and various types of head and neck cancer, larynx cancer, oral cavity cancer, urothelial cancer, KRAS mutant tumors, Myelodysplastic syndromes (MDS), as well as B-cell malignancies, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; adult T-cell leukemia/lymphoma; myeloma; multiple myeloma and post-transplant lymphoproliferative disorder (PTLD), lymphoid malignancies, abnormal vascular proliferation associated with phakomatoses, edema (such as that associated with brain tumors), and Meigs' syndrome, rectal cancer, renal cell cancer, soft-tissue sarcoma, Kaposi's sarcoma, carcinoid carcinoma, ovarian early or advanced (including metastatic).

In some embodiments, the cancer is selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS).

In some embodiments, the cancer is selected from the group consisting of advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer. In some embodiments, cancer treatment occurs due to NK-cell activation. In some embodiments, the NK-cell activation level is as compared to a control or standard level of NK-cell activation or as compared to unactivated NK-cells level.

As shown in the Examples of WO2016/134333, PVRIG is over expressed and/or correlates with tumor lymphocyte infiltration (as demonstrated by correlation to CD3, CD4, CD8 and PD-1 expression) in a number of different tumors of various origins, and thus is useful in treating any cancer, including but not limited to, prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

In particular, anti-PVRIG antibodies comprising CHA.7.518.1 as the PVRIG binding portion may find use in treating prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS) In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level. In particular, anti-PVRIG antibodies comprising CHA.7.518.1 as the PVRIG binding portion may find use in treating advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

In particular, anti-PVRIG antibodies comprising CHA.7.538.1.2 as the PVRIG binding portion may find use in treating prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS). In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

In particular, anti-PVRIG antibodies comprising CHA.7.538.1.2 as the PVRIG binding portion may find use in treating advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

In particular, anti-PVRIG antibodies comprising CHA.7.518.4 as the PVRIG binding portion may find use in treating prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS). In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

In particular, anti-PVRIG antibodies comprising CHA.7.518.4 as the PVRIG binding portion may find use in treating advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

In particular anti-TIGIT antibodies comprising CPA.9.086 as the TIGIT binding portion may find use in treating prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS). In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

In particular anti-TIGIT antibodies comprising CPA.9.086 as the TIGIT binding portion may find use in treating advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

In particular, anti-TIGIT antibodies comprising CPA.9.083 as the TIGIT binding portion may find use in treating prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS). In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

In particular, anti-TIGIT antibodies comprising CPA.9.083 as the TIGIT binding portion may find use in treating advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

In particular, anti-TIGIT antibodies comprising CHA.9.547.7 as the TIGIT binding portion may find use in treating prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS). In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

In particular, anti-TIGIT antibodies comprising CHA.9.547.13 as the TIGIT binding portion may find use in treating prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS). In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

In particular, anti-TIGIT antibodies comprising CHA.9.547.13 as the TIGIT binding portion may find use in treating advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

In particular, anti-TIGIT antibodies comprising CPA.9.547.18 as the TIGIT binding portion may find use in treating prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS). In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

In particular, anti-TIGIT antibodies comprising CPA.9.547.18 as the TIGIT binding portion may find use in treating advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

In some embodiments, the cancer is selected from the group consisting of triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, myeloma and Myelodysplastic syndromes (MDS). In some embodiments, cancer treatment occurs due to NK-cell activation. In some embodiments, the NK-cell activation level is as compared to a control or standard level of NK-cell activation or as compared to unactivated NK-cells level.

1. AML Tumor Properties for Treatment

In some embodiments, the cancer for treatment is AML. The French American British (FAB) classification system was used from 1976 to 2001 and divided AML into M0-M7 (Br J Haematol 1976; 33:451). The WHO classification (2001 and revised in 2008) requires minimium of 20% of blasts in bone marrow or blood to diagnose AML (was 30% under FAB) and eliminates myelodysplastic category of “refractory anemia with excess blasts in transformation” (Blood 2002; 100:2292). The WHO classification also separates out AML “with recurrent genetic abnormalities” which have distinct clinical features.

In some embodiments, the individual to be treated or for which NK-cell activation finds use is an individual that has AML cancer cells that are PVRL2^{hi}PVRlow and/or PVRL2⁺PVR^{low}. In some embodiments, the individual to be treated or for which NK-cell activation finds use is an individual that has AML cancer cells that are PVRL2^{hi}PVRlow. In some embodiments, the individual to be treated or for which NK-cell activation finds use is an individual that has AML cancer cells that are PVRL2⁺PVR^{low}. In some embodiments, the AML cancer cells are PVRL2^{hi}PVR^{low} and/or PVRL2⁺PVR^{low}. In some embodiments, the AML cancer cells are PVRL2^{hi}PVR^{low}. In some embodiments, the AML cancer cells are PVRL2⁺PVR^{low}.

In some embodiments, the level of PVRL2 and/or PVR expression are determined by measuring the level of PVRL2 and/or PVR using immunohistochemistry and appropriate staining procedures. In some embodiments, the level of PVRL2 and/or PVR expression are determined by measuring the level of PVRL2 and/or PVR using FACS analyses. In some embodiments, the level of PVRL2 and/or PVR expression, including the level of PVRL2 and/or PVR expression, is scored by a board-certified pathologist.

In some embodiments, PVRL2 status is determined based on expression level. In some embodiments, the presence of any PVRL2 indicates PVRL2+. In some embodiments, expression is based on tumor membrane staining and the presence if any tumor membrane staining for PVRL2 indicates PVRL2⁺.

In some embodiments, PVRL2^{hi} status is determined based on expression level. In some embodiments, PVRL2^{hi} status is expression is based on tumor membrane staining. In some embodiments, the PVRL2^{hi} status is indicated by at least 20%, at least 30%, at least 40%, or at least 50% tumor membrane staining.

In some embodiments, PVR^{low} status is determined based on expression level. In some embodiments, PVR^{low} status is expression is based on tumor membrane staining. In some embodiments, the PVR^{low} status is indicated by 0%, less that 1%, less than 2% or less than 5% tumor membrane staining.

In some embodiments, the AML cancer cells are AML blasts.

In some embodiments, the AML is selected from the group consisting of AML with minimal differentiation (M0), AML without maturation (M1), AML with maturation (M2), Acute Promeyelocitic Leukemia (M3), Acute myelomonocytic leukemia (M4), Acute monoblastic/monocytic leukemia (M5a/b), Acute Erythroleukemia (M6), Acute Megakaryocytic Leukemia (M7), Acute basophilic leukemia, Acute panmyelosis with myelofibrosis, therapy related AML (Alkylating agent related AML or Topoisomerase II inhibitor related AML), AML with myelodysplasia related changes (AMLMRC), AML with myelodysplasia related changes, myeloid sarcoma, myeloid proliferations related to Down syndrome (transient abnormal myelopoeisis or myeloid leukemia associated with Down syndrome), blastic plasmacytoid dentritic cell neoplasm, acute leukemia of ambiguous lineage, and AML with recurrent genetic abnormalities.

In some embodiments, the acute leukemia of ambiguous lineage is selected from the group consisting of acute undifferentiated leukemia, mixed phenotype acute leukemia with t(9;22)(q34;q11.2) (BCR-ABL1), mixed phenotype acute leukemia with t(v;11q23) (MLL rearranged), mixed phenotype acute leukemia (B/myeloid, NOS), mixed phenotype acute leukemia (T/myeloid, NOS), mixed phenotype acute leukemia (NOS, rare types), and other acute leukemia of ambiguous lineage.

In some embodiments, the AML with recurrent genetic abnormalities is selected from the group consisting of AML with t(8;21)(q22;q22) (RUNX1-RUNX1T1), AML with inv(16)(p13.1;q22) or t(16;16)(p13.1;q22) (CBF&beta-MYH11), Acute promyelocytic leukemia with t(15;17)(q22;q12) (PML/RAR&alpha and variants), AML with t(9;11)(p22;q23) (MLLT3-MLL), AML with t(6;9)(p23;q34) (DEK-NUP214), AML with inv(3)(q21q26.2) or t(3;3)(q21;q26.2) (RPN1-EVI1), AML (megakaryoblastic) with t(1;22)(p13;q13) (RBM15-MKL1), AML with mutated NPM1, and AML with mutated CEBPA.

In some embodiments, the AML is related to specific mutations in one or more genes that are selected from the group consisting of FLT3, NPM1, IDH1/2, DNMT3A, KMT2A, RUNX1, ASXL, and TP53.

### C. Combination Therapies

Combination therapies comprising one or more therapeutic anti-PVRIG antibodies and one or more therapeutic anti-TIGIT antibodies plus an additional therapeutic agent, specific for the disease condition, are contemplated. For example, in the area of immunotherapy, there are a number of promising combination therapies using a chemotherapeutic agent (either a small molecule drug or an anti-tumor antibody) or with an immuno-oncology antibody.

The terms “in combination with” and “co-administration” are not limited to the administration of the prophylactic or therapeutic agents at exactly the same time. Instead, it is meant that the antibody and the other agent or agents are administered in a sequence and within a time interval such that they may act together to provide a benefit that is increased versus treatment with only either the antibody of the present invention or the other agent or agents. It is preferred that the antibody and the other agent or agents act additively, and especially preferred that they act synergistically. Such molecules are suitably present in combination in amounts that are effective for the purpose intended. The skilled medical practitioner can determine empirically, or by considering the pharmacokinetics and modes of action of the agents, the appropriate dose or doses of each therapeutic agent, as well as the appropriate timings and methods of administration.

Accordingly, the anti-PVRIG and anti-TIGIT antibodies of the present invention are administered concomitantly with one or more other therapeutic regimens or agents. The additional therapeutic regimes or agents may be used to improve the efficacy or safety of the anti-PVRIG and anti-TIGIT antibodies, in particular as it relates to NK-cell activation and enhanced tumor killing by NK-cells. Also, the additional therapeutic regimes or agents may be used to treat the same disease or a comorbidity rather than to alter the action of the anti-PVRIG and anti-TIGIT antibodies. For example, an anti-PVRIG and anti-TIGIT, antibodies of the present invention may be administered to the patient along with chemotherapy, radiation therapy, or both chemotherapy and radiation therapy.

1 PVRIG and TIGIT Antibodies with Chemotherapeutic Small Molecules

The anti-PVRIG and anti-TIGIT antibodies of the present invention may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, or other therapeutic agents.

In this context, a “chemotherapeutic agent” is a chemical compound useful in the treatment of cancer. Examples of chemotherapeutic agents include alkylating agents such as thiotepa and cyclosphosphamide, alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL′); beta-lapachone; lapachol; colchicines; betulinic acid; a camptothecin (including the synthetic analogue topotecan (HYCAMTN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®; Bristol-Myers Squibb Oncology, Princeton, N.J.), ABRAXANE®, cremophor-free, albumin-engineered nanoparticle formulation of paclitaxel (American Pharmaceutical Partners, Schaumberg, Ill.), and docetaxel (TAXOTERE®; Rhone-Poulenc Rorer, Antony, France); chloranbucil; gemcitabine (GEMZARM®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone; CVP, an abbreviation for a combined therapy of cyclophosphamide, vincristine, and prednisolone; and FOLFOX, an abbreviation for a treatment regimen with oxaliplatin (ELOXATIN®) combined with 5-FU and leucovorin.

According to at least some embodiments, the anti-PVRIG and anti-TIGIT antibodies for use with the invention could be used in combination with any of the known in the art standard of care cancer treatment (as can be found, for example, in http://www.cancer.gov/cancertopics).

Thus, in some cases, the anti-PVRIG antibodies outlined herein (particularly those including CHA.7.538.1.2 and/or CHA.7.518.1) can be combined with chemotherapeutic agents. Similarly, the anti-TIGIT antibodies outlined herein (particularly those including CPA.9.086, CPA.9.083 and/or CHA.9.547.13) can be combined with chemotherapeutic agents.

### D. Assessment of Treatment

Generally, the anti-PVRIG and anti-TIGIT antibodies for use with the invention, are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays. For example, evaluation of changes in immune status (e.g. presence of ICOS+CD4+ T cells following ipi treatment) along with “old fashioned” measurements such as tumor burden, size, invasiveness, LN involvement, metastasis, etc. can be done. Thus, any or all of the following can be evaluated: the inhibitory effects of PVRIG or TIGIT on CD4⁺ T cell activation or proliferation, CD8⁺ T (CTL) cell activation or proliferation, CD8⁺ T cell-mediated cytotoxic activity and/or CTL mediated cell depletion, NK cell activity and NK mediated cell depletion, the potentiating effects of PVRIG or TIGIT on Treg cell differentiation and proliferation and Treg- or myeloid derived suppressor cell (MDSC)-mediated immunosuppression or immune tolerance, and/or the effects of PVRIG or TIGIT on proinflammatory cytokine production by immune cells, e.g., IL-2, IFN-γ or TNF-α production by T or other immune cells. In some embodiments, NK cell activity and/or NK mediated cell depletion are evaluated. In some embodiments, NK cell tumor killing activity is evaluated.

In some embodiments, assessment of treatment is done by evaluating immune cell proliferation, using for example, CFSE dilution method, Ki67 intracellular staining of immune effector cells, and 3H-Thymidine incorporation method.

In some embodiments, assessment of treatment is done by evaluating the increase in gene expression or increased protein levels of activation-associated markers, including one or more of: CD25, CD69, CD137, ICOS, PD1, GITR, OX40, and cell degranulation measured by surface expression of CD107A.

In some embodiments, the assessment of treatment is done by assessing the amount of T cell proliferation in the absence of treatment, for example prior to administration of the antibodies for use with the invention. If, after administration, the patient has an increase in T cell proliferation, e.g. a subset of the patient's T cells are proliferating, this is an indication that the T cells were activated.

In some embodiments, the assessment of treatment is done by assessing the amount of T cell proliferation in the absence of treatment, for example prior to administration of the antibodies for use with the invention. If, after administration, the patient has an increase in NK-cell proliferation, e.g. a subset of the patient's NK-cells are proliferating, this is an indication that the NK-cells were activated. In some embodiments, NK cell activation is evaluated by the expression of cell-surface activation markers, e.g., CD69, CD107a and/or CD137, as well as by IFNγ secretion. In some embodiments, NK cell activation is evaluated by the expression of CD69. In some embodiments, NK cell activation is evaluated by the expression CD107a. In some embodiments, NK cell activation is evaluated by the expression of CD137. In some embodiments, NK cell activation is evaluated by IFNγ secretion. In some embodiments, the cytolytic capacity of NK cells towards different tumor cell lines serves as an additional NK cell activation readout. In some embodiments, assessment of treatment with the antibodies for use with the invention can be done by measuring the patient's IFNγ levels prior to administration and post-administration to assess efficacy of treatment. This may be done within hours or days.

In general, gene expression assays are done as is known in the art. See for example Goodkind et al., Computers and Chem. Eng. 29(3):589 (2005), Han et al., Bioinform. Biol. Insights 11/15/15 9(Suppl. 1):29-46, Campo et al., Nod. Pathol. 2013 January; 26 suppl. 1:S97-S110, the gene expression measurement techniques of which are expressly incorporated by reference herein.

In general, protein expression measurements are also similarly done as is known in the art, see for example, Wang et al., Recent Advances in Capillary Electrophoresis-Based Proteomic Techniques for Biomarker Discovery, Methods. Mol. Biol. 2013:984:1-12; Taylor et al, BioMed Res. Volume 2014, Article ID 361590, 8 pages, Becerk et al., Mutat. Res 2011 June 17:722(2): 171-182, the measurement techniques of which are expressly incorporated herein by reference.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, ⁵¹Cr or ³⁵S release method, LDH activity, MTT and/or WST assays, Calcein-AM assay, Luminescent based assay, and others.

In some embodiments, assessment of treatment can be further evaluated by assessing T cell activity measured by cytokine production, measure either intracellularly in culture supernatant using cytokines including, but not limited to, IFNγ, TNFα, GM-CSF, IL2, IL6, IL4, IL5, IL10, IL13 using well known techniques.

Accordingly, assessment of treatment can be done using assays that evaluate increases in NK and/or NKT cell activity. In some embodiments, assessment of treatment can be done using assays that further evaluate one or more of the following: (i) increases in immune response, (ii) increases in activation of αβ and/or γδ cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs).

### E. Assays to Measure Efficacy

In some embodiments, T cell activation is assessed using a tumor cell killing assay as is described in Example 1. An increase in activity indicates immunostimulatory activity. Appropriate increases in activity are outlined below.

In one embodiment, the signaling pathway assay measures increases or decreases in immune response as measured for an example by phosphorylation or de-phosphorylation of different factors, or by measuring other post translational modifications. An increase in activity indicates immunostimulatory activity. Appropriate increases in activity are outlined below.

In one embodiment, the signaling pathway assay measures increases or decreases in activation by proliferation or by changes in expression of activation markers like for an example CD69 and/or CD107a. An increase in activity indicates immunostimulatory activity. In some meodimetns, the signaling pathway assay measure NK cell activation. In some embodiments for NK cells, increases in proliferation, cytotoxicity (ability to kill target cells and increases CD107a, granzyme, and perforin expression), cytokine production (e.g., IFNγ and TNF), and cell surface receptor expression (e.g., CD25) would be indicative of immune modulation that would be consistent with enhanced killing of cancer cells. In some embodiments, NK cell activation is evaluated by the expression of cell-surface activation markers, e.g., CD69, CD107a and/or CD137, as well as by IFNγ secretion. In some embodiments, NK cell activation is evaluated by the expression of CD69. In some embodiments, NK cell activation is evaluated by the expression CD107a. In some embodiments, NK cell activation is evaluated by the expression of CD137. In some embodiments, NK cell activation is evaluated by IFNγ secretion. In some embodiments, assessment of treatment with the antibodies for use with the invention can be done by measuring the patient's IFNγ levels prior to administration and post-administration to assess efficacy of treatment. In some embodiments, the cytolytic capacity of NK cells towards different tumor cell lines serves as an additional NK cell activation readout. In one embodiment, the signaling pathway assay measures increases or decreases in NK and/or NKT cell activity as measured for an example by direct killing of target cells like for an example cancer cells or by cytokine secretion or by changes in expression of activation markers like for an example CD69, CD107a and/or CD137, as well as by IFNγ secretion. An increase in activity indicates immunostimulatory activity. Appropriate increases in activity are outlined below.

In some embodiments, to evaluate the cytolytic capacity of NK cells, target cell are labeled by a fluorescent dye and then enumerated by flow cytometry prior to and following co-culture with NK cells. In some alterntive embodiments, target cells can be loaded with a ligand that is released to the culture medium when the membrane is compromised (e.g., following attack by NK cells), the released ligand forms a highly fluorescent chelate with an added substrate. In these embodiments, the florescence is then quantified by an automated fluorescence (or luminescence) reader.

Appropriate increases in activity or response (or decreases, as appropriate as outlined above), are increases of at least about 5%, 10%, 20%, 30%, 40%, 50%, 60%, 70%, 80%, 90%, 95% or 98 to 99% percent over the signal in either a reference sample or in control samples, for example test samples that do not contain an anti-PVRIG antibody and/or an anti-TIGIT antibody of the invention. In some embodiments, assessment of treatment with the antibodies for use with the invention can be done by measuring the patient's activity or response levels prior to administration and post-administration to assess efficacy of treatment.

In some embodiments, increases of at least one-, two-, three-, four- or five-fold post-administration of the anti-PVRIG and anti-TIGIT antibodies as compared to reference or control samples show efficacy. In some embodiments, increases of at least one-, two-, three-, four- or five-fold post-administration as compared to prior to administration of the anti-PVRIG and anti-TIGIT antibodies show efficacy.

### X. EXAMPLES

### Example 1: Activation of Human NK Cells Modulates Expression of the Inhibitory Receptor PVRIG

### 1. Introduction

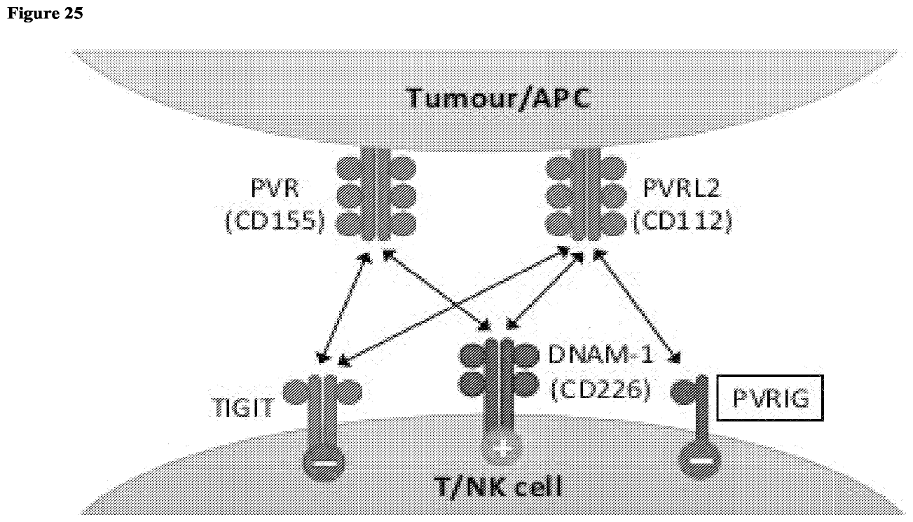

Poliovirus receptor-related immunoglobulin domain-containing (PVRIG) is an immune checkpoint molecule expressed on T and NK cells (1,2). PVRIG inhibits effector cell function upon binding to poliovirus receptor-related 2 (PVRL2) (1-3), an adhesion molecule that is overexpressed in some cancers. PVRL2 also binds another inhibitory receptor, T cell immunoreceptor with Ig and ITIM domains (TIGIT), as well as the activating receptor DNAX accessory molecule-1 (DNAM-1) (4, FIG. 25).

This study aimed to investigate the role of PVRIG in regulating human NK cell function.
Determine whether blocking PVRIG enhances killing of tumour cells by healthy donor PBMCs
Assess the expression of PVRL2 and PVR in primary bone marrow (BM) samples from acute myeloid leukemia (AML) patients
Assess the expression of PVRIG, TIGIT and DNAM-1 after in vitro co-culture of NK cells with activatory stimuli

### PVRIG Blockade Enhances NK Cell Killing of Tumour Cell Lines

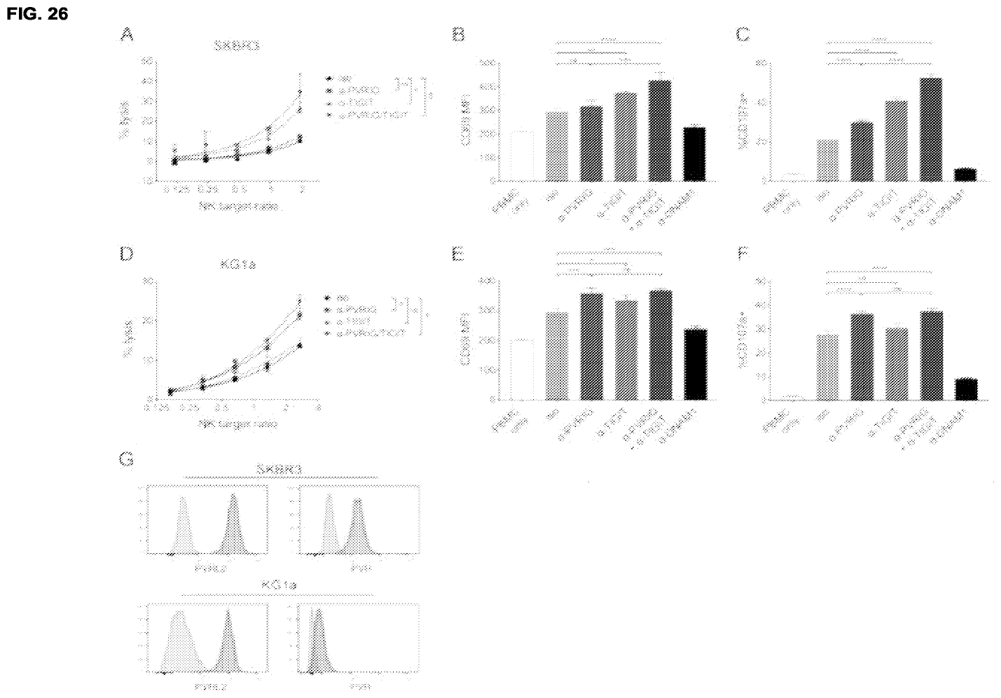

FIG. 26. A-F) Healthy donor PBMCs were co-cultured with A-C) SKBR3 or D-F) KG1a in the presence of the indicated blocking antibodies. A,D) Lysis of target cells and expression of B,E) CD69 and C,F) CD107a on NK cells was assessed after 4 hr. G) Expression of PVRL2 or PVR (red) on SKBR3 and KG1a cells compared with isotype control stain (grey).

### PVRIG and PVRL2 are Expressed in AML Patient Bone Marrow

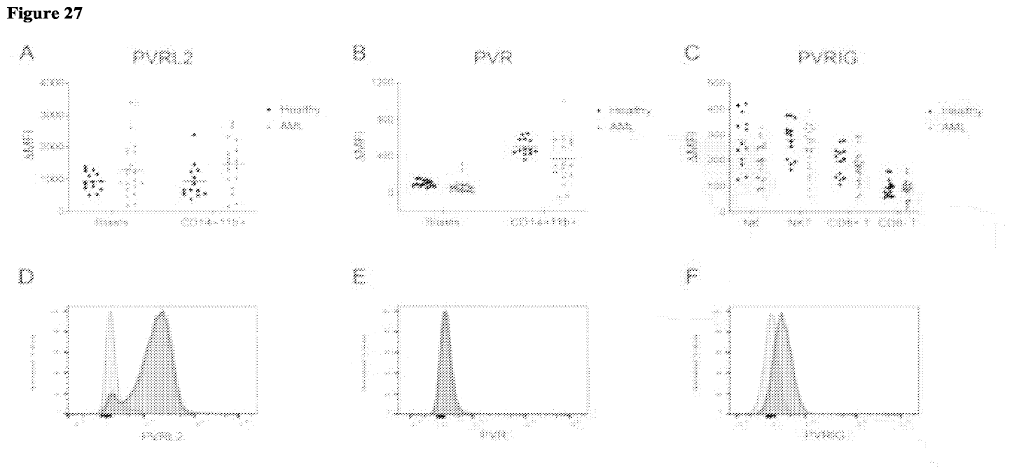

FIG. 27. A-C) Expression of A) PVRL2 B) PVR or C) PVRIG on blasts or immune cell types in the bone marrow of AML patients (n=19-20) or healthy donors (n=13). D-F) Representative histograms of D) PVRL2 on AML blasts E) PVR on AML blasts or F) PVRIG on NK cells in the bone marrow of an AML patient. Histograms of test (red) and isotype control stains (blue) are shown.

### PVRIG Expression on NK Cells is Decreased Upon Activation

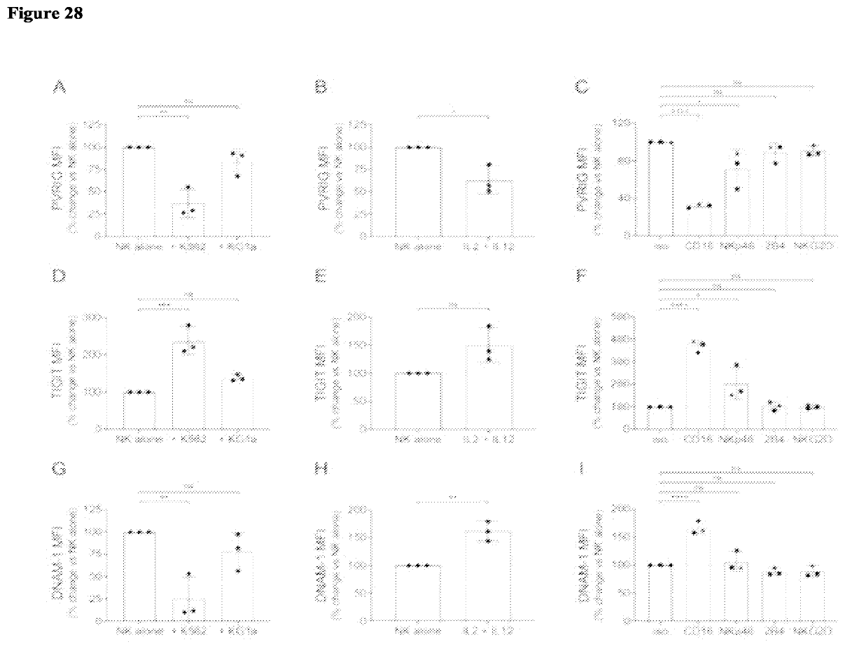

FIG. 28. Expression of A-C) PVRIG D-F) TIGIT or G-I) DNAM-1 on isolated NK cells after 24 hr co-culture with tumour cells, or 24 hr stimulation with the indicated cytokines or agonistic antibodies. Percentage change in MFI relative to NK alone is shown.

PVRIG is Constitutively Recycled from NK Cell Surface

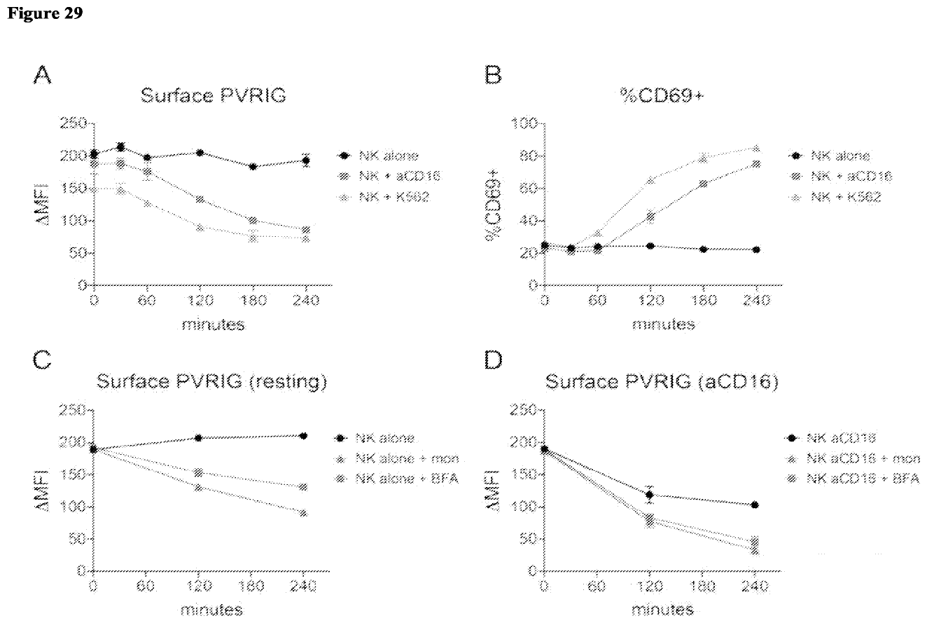

FIG. 29. Expression of A,C,D) PVRIG and B) CD69 on isolated NK cells incubated alone, with K562 cells, or with plate-bound α-CD16 antibody at 37° C. for the indicated time points, in the presence or absence of monensin (mon) or brefeldin A (BFA).

### Conclusion

PVRIG blockade enhances killing of PVRL2+ tumour cells by NK cells in vitro.

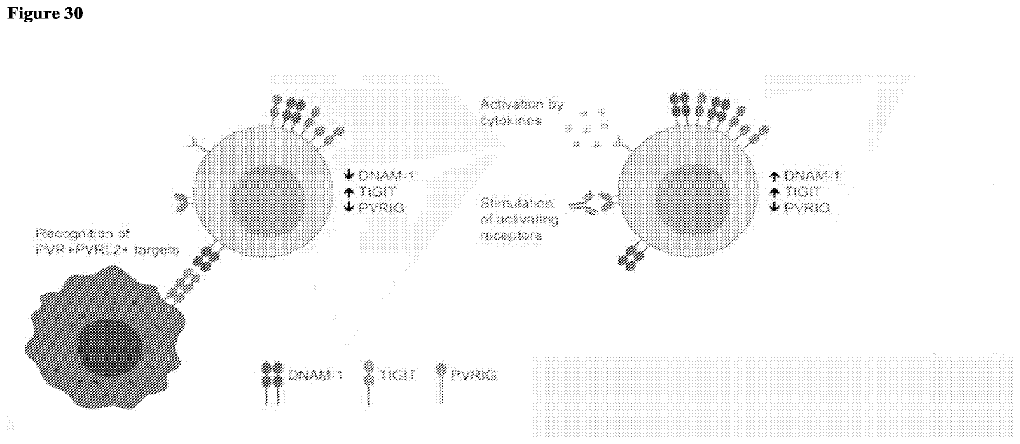

Recognition of targets or activation of NK cells via cytokines or agonistic receptors modulates PVRIG/TIGIT/DNAM-1 expression, as in FIG. 30 (Modulation of DNAM-1/TIGIT/PVRIG on NK cells upon activation).

Constitutive recycling of PVRIG suggests that a greater amount of PVRIG is available to be blocked over time than can be observed at a single time point.

Thus, although NK cells in AML patients do not express higher levels of PVRIG than healthy donors, PVRIG blockade may still be effective, particularly as AML blasts express high levels of PVRL2.

### REFERENCES

¹ Zhou, Y. A. Patriccia, A. C. Shulick, W. Chen, M. R. Koenig, J. T. Byers, S. Yao, S. Bevers, and B. H. Edit. 2016. Identification of CD1128 as a novel checkpoint for human T cells. J. Exp. Med. 213: 177-176.
Xu, F. A. Sunderland, Y. Zhou, R. D. Schulick, B. H. Edit, and Y. Zhu. 2017 Blockade of CD1128 and TIGIT signaling sensitizes human natural killer cell functions. Cancer Immunol. Immunother. 66: 1367-1375.
³ Whelan, S., E. Ophir, M. F. Kotturi, O. Levy, S. Ganguly, L. Leung, I. Vaknin, S. Kumar, L. Dassa, K. Hansen, D. Bernados, B. Murter, A. Soni, J. M. Taube, A. N. Fader, T. L. Wang, I. M. Shih, M. White, D. M. Pandoil, and S. C. Liang. 2019. PVRIG and PVRL2 Are Induced in Cancer and Inhibit (CD8(+) T-cell Function. Cancer Immunol. Res. 7: 257-268.
⁴ Sanchez-Correa, B., I. Valhondo, F. Hassouneh, N. Lopez-Sejas, A. Pera, J. M. Bergua, M. J. Arcos, H. Banas, I. Casas-Aviles, E. Duran, C. Alonso, R. Solana, and R. Tarazona. 2019. DNAM-1 and the TIGIT/PVRIG/TACTILE Axls: Novel Immune Checkpoints for Natural Killer Cell-Based Cancer Immunotherapy Cancers (Basel) 11.

### Example 2: PVRIG is a Novel NK Cell Immune Checkpoint Receptor in Acute Myeloid Leukemia

### Abstract

This study explored the novel immune checkpoint poliovirus receptor-related immunoglobulin domain-containing (PVRIG) in acute myeloid leukemia (AML). We showed that AML patient blasts consistently expressed the PVRIG ligand (poliovirus receptor-related 2, PVRL2). Furthermore, PVRIG blockade significantly enhanced NK cell killing of PVRL2⁺, poliovirus receptor (PVR)^{lo} ML cell lines, and significantly increased NK cell activation and degranulation in the context of patient primary AML blasts. However, in AML patient bone marrow, NK cell PVRIG expression levels were not increased. To understand how PVRIG blockade might potentially be exploited therapeutically, we investigated the biology of PVRIG and revealed that NK cell activation resulted in reduced PVRIG expression on the cell surface. This occurred whether NK cells were activated by tumour cell recognition, cytokines (IL-2 and IL-12) or activating receptor stimulation (CD16 and NKp46). PVRIG was present at higher levels in the cytoplasm than on the cell surface, particularly on CD56^{bright} NK cells, which further increased cytoplasmic PVRIG levels following IL-2 and IL-12 activation. PVRIG was continually transported to the cell surface via the endoplasmic reticulum (ER) and Golgi in both unstimulated and activated NK cells. Taken together, our findings suggest that anti-PVRIG blocking antibody functions by binding to surface-bound PVRIG, which undergoes rapid turnover in both unstimulated and activated NK cells. We conclude that the PVRIG-PVRL2 immune checkpoint axis can feasibly be targeted with PVRIG blocking antibody for NK-mediated immunotherapy of PVRL2⁺ AML.

### Introduction

Poliovirus receptor-related immunoglobulin domain-containing (PVRIG) has recently been identified as an immune checkpoint molecule with potential for therapeutic development.¹ In humans, PVRIG is expressed on T cells (predominantly CD8⁺ T cells) and NK cells, but not on B cells, monocytes or neutrophils.¹ PVRIG binds to a single ligand, poliovirus receptor-related 2 (PVRL2, also known as CD112 or Nectin-2), and exerts an inhibitory effect on cytotoxic lymphocyte activity, likely via an ITIM-like motif in its intracellular domain. ¹⁻³ PVRL2 is an adhesion molecule involved in the formation of cell-cell junctions, and is overexpressed in various cancers.⁴⁻⁸ PVRL2 is also a ligand of the co-activating receptor DNAX accessory molecule 1 (DNAM-1)^{9, 10} and weakly binds another inhibitory receptor, T cell immunoreceptor with Ig and ITIM domains (TIGIT).¹¹⁻¹³ Recently, Whelan et al. demonstrated the inhibitory effect of PVRL2 was predominantly mediated by PVRIG and not TIGIT.³ DNAM-1 and TIGIT (but not PVRIG) also bind to a closely related molecule, poliovirus receptor (PVR, also known as CD155 or Necl-5).^{9, 11, 12} Competition studies have determined that PVR has higher affinity for TIGIT than DNAM-1, and PVRL2 has a higher affinity for PVRIG than DNAM-1, suggesting that the inhibitory signal is dominant.^{1,11}

PVRIG inhibitory function was shown using anti-PVRIG blocking antibodies. Xu et al. demonstrated that PVRIG blocking antibodies significantly increased NK cell cytotoxicity against breast cancer cell lines in vitro, an effect that was further enhanced when used in combination with TIGIT blocking antibodies.² An independent group using a different anti-PVRIG antibody similarly showed that PVRIG blockade enhanced T cell cytotoxicity against melanoma and pancreatic cancer cell lines, which was also augmented by combination with TIGIT blockade.³ Notably, Whelan et al. demonstrated that T cells isolated from patient tumours and activated via CD3 increased interferon gamma production in response to combination PVRIG/TIGIT blockade.³ PVRIG blockade also reduced tumour burden in a mouse model when combined with anti-PDL1.¹⁴ On the basis of these data, a human IgG4 anti-PVRIG blocking antibody is currently undergoing phase I clinical trials in patients with advanced solid tumours.¹⁵

As PVRIG is present on both T cells and NK cells, blocking PVRIG provides the opportunity to augment both major cytotoxic effector cell types. Although many studies focused on the capacity for immune checkpoint blockade to enhance T cell responses, the contribution of NK cells should not be overlooked. For instance, tumours often downregulate human leukocyte antigen (HLA) class I to evade CD8⁺ T cell recognition.¹⁶ However, this simultaneously removes the ligand for killer cell immunoglobulin-like receptors (KIRs) on NK cells, rendering tumours more sensitive to NK cell-mediated killing.¹⁷ Reducing the inhibitory signal from KIRs has also been shown to be effective in controlling acute myeloid leukemia (AML). AML is an aggressive disease in which myeloid progenitor cells proliferate uncontrollably, and which is frequently treated with allogeneic hematopoietic stem cell transplant (allo-HSCT) when patients relapse after front-line chemotherapy. In a seminal study of allo-HSCT patients, mismatches between KIRs on donor NK cells and recipient HLA was a key predictor of survival.^{18, 19} Recipients lacking HLA ligands for one or more of the KIRs expressed by the donor experienced graft-versus-host NK alloreactivity, which was significantly associated with a lower relapse rate.^{19,20}

Given the pivotal role of NK cells in AML, strategies to enhance NK cell activity could provide significant benefit for patients with AML, who have a 5-year survival rate of less than 30% with current treatments.′ This study aimed to determine whether PVRIG blockade could be used to enhance NK cell responses against AML. Using healthy donor and AML patient blood or bone marrow samples, we evaluated the expression of PVRIG and PVRL2 on NK cells and AML blasts respectively. We also investigated whether PVRIG blockade could enhance NK cell-mediated killing of AML blasts, and the kinetics of PVRIG surface expression to reveal when the target is expressed following AML target cell activation of NK cells.

### Methods

### Reagents and Cell Lines

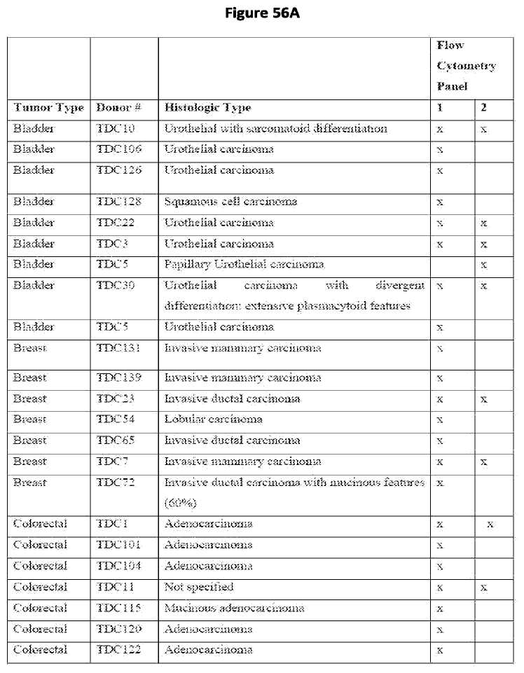

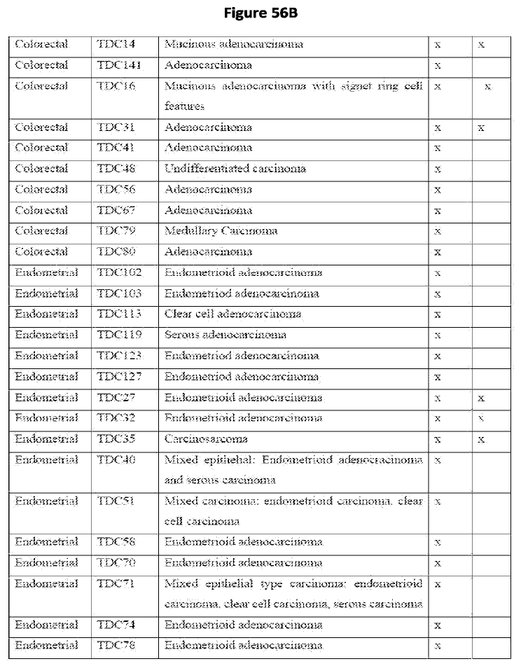

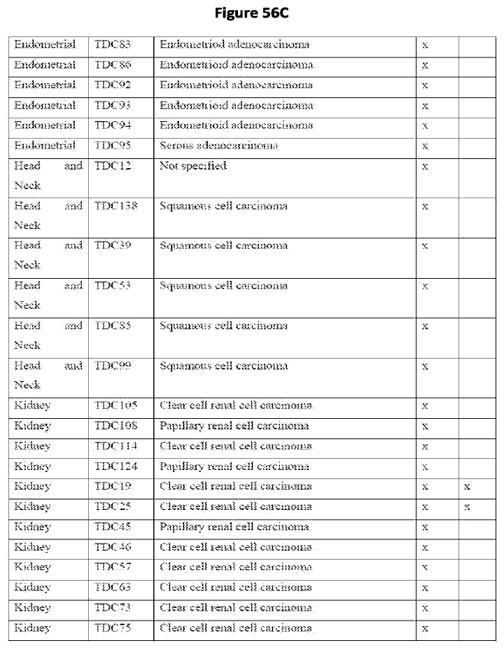

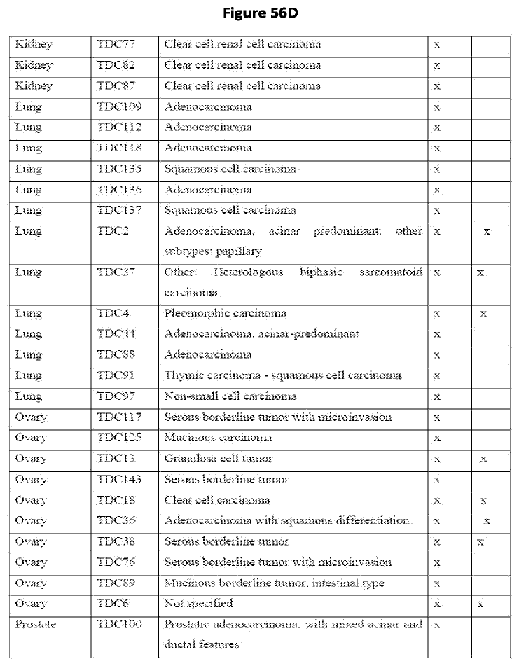

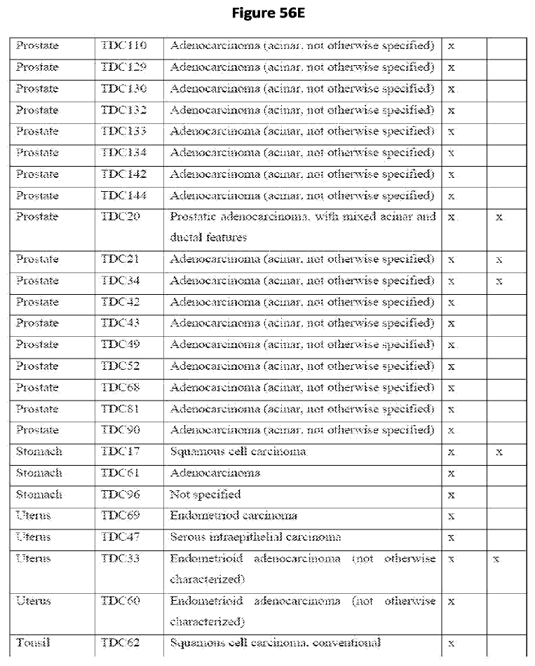

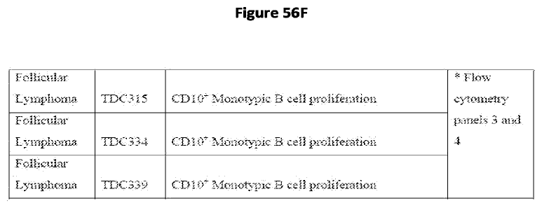

Anti-PVRIG and anti-TIGIT blocking antibodies were provided by Compugen, USA, Inc. Anti-DNAM-1 (11A8), anti-CD16 (3G8), anti-NKp46 (9E2), anti-2B4 (eBioC1.7) and anti-NKG2D (1D11) purified antibodies were purchased from Biolegend. Recombinant human IL-2, IL-12, IL-15 and IL-18 were purchased from Peprotech. Monensin (GolgiStop, BD Biosciences) and brefeldin A (eBioscience) were both used at 1:1000. Antibodies used for flow cytometry staining are listed in FIG. 56. SKBR3, KG1a, K562, ML-2, THP-1 and Kasumi-1 cell lines were maintained in RPMI 1640 (Gibco) supplemented with Glutamax, penicillin, streptomycin and 10% (or 20% for Kasumi-1) fetal calf serum (FCS). AML-193 cell line was maintained in Iscove's Modified Dulbecco's Media supplemented with 5% FCS, 5 μg/ml transferrin, 5 μg/ml insulin and 2 ng/ml GM-CSF. All cell lines tested negative for mycoplasma.

### AML Patient and Healthy Donor Bone Marrow Samples

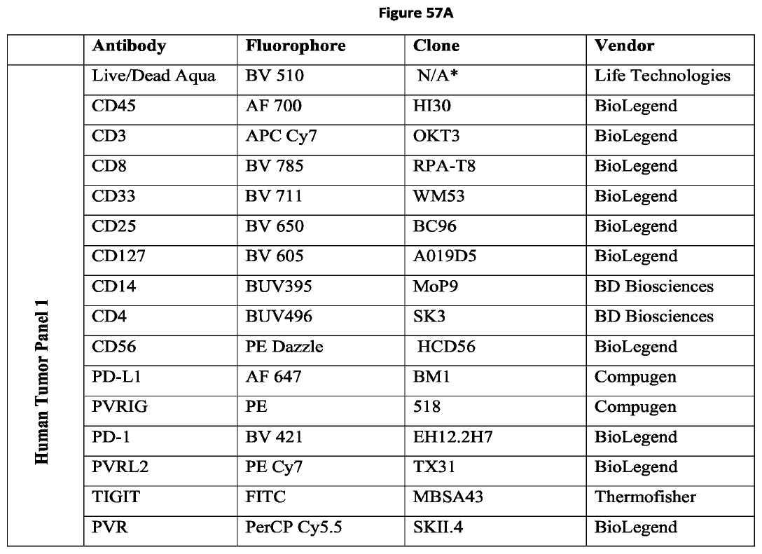

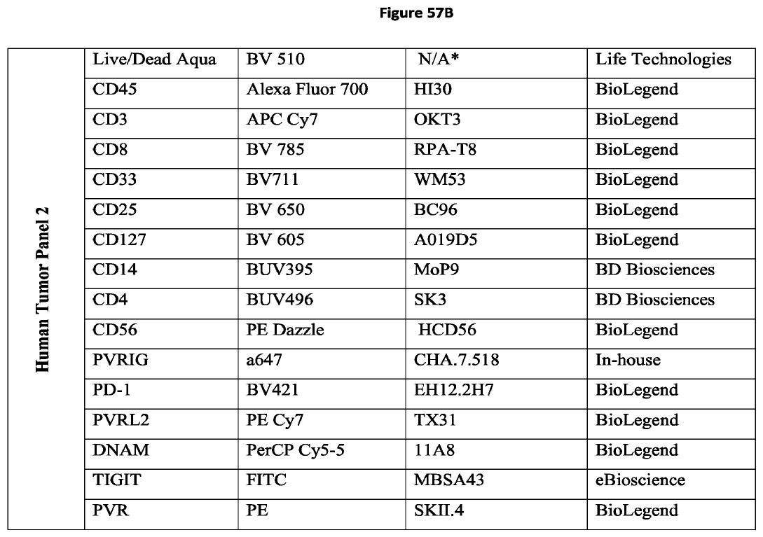

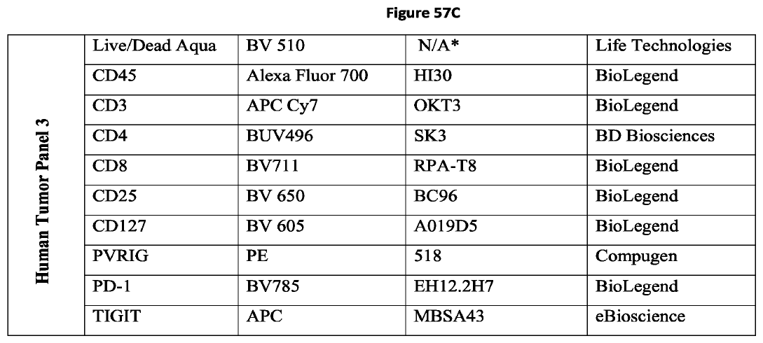

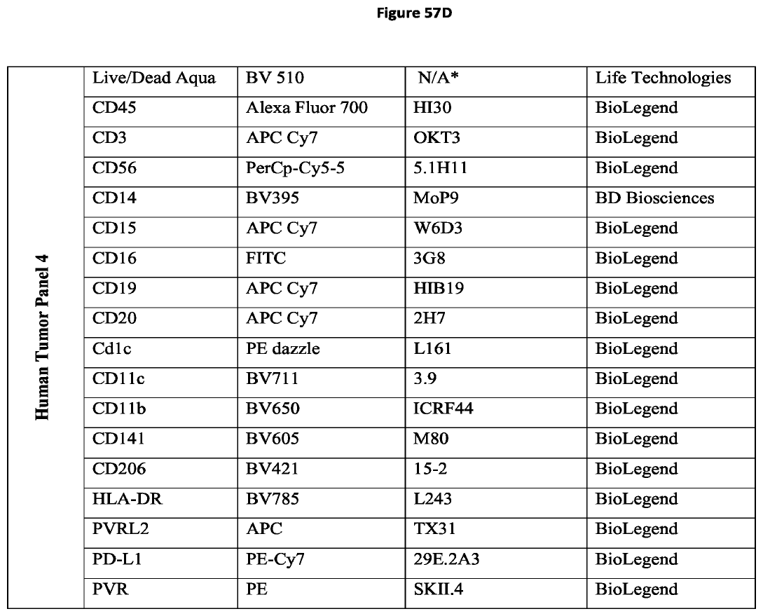

All patient and healthy donor samples were obtained under ethics approval from the Peter MacCallum Cancer Centre human ethics committee (HREC approval numbers 01/14 and 10-61). Cryopreserved AML patient diagnostic bone marrow samples were obtained from the Cancer Collaborative Biobank (Metro South Health, Queensland, Australia). Patient clinical characteristics are summarised in FIG. 57. Healthy donor bone marrow samples were obtained from Royal Melbourne Hospital (Melbourne, Australia) or purchased from AllCells (Alameda, Calif.). All bone marrow samples were used for flow cytometry staining immediately after thawing.

### Healthy Donor PBMC and NK Cells

Peripheral blood mononuclear cells (PBMCs) were isolated from healthy donor buffy coats (Australian Red Cross Blood Service) by density gradient (Ficoll-Paque, GE Healthcare Life Science) and cryopreserved. One day prior to experiments, PBMCs were thawed and treated with DNase I (Merck) for 15 min at 37° C. Where required, NK cells were isolated by negative selection using a human NK Cell Isolation Kit (Miltenyi Biotec) according to manufacturer's instructions (except antibodies and beads were used at half the recommended concentration). The purity of NK cells as determined by flow cytometry was >95%. Bulk PBMCs or isolated NK cells were incubated in media containing 25 U/ml IL-2 overnight at 37° C. before use in assays.

### NK Cell Stimulation

Isolated NK cells were incubated at 37° C. alone, with the specified combination of cytokines, with target cells at a 1:1 ratio, or in wells pre-coated (overnight 4° C.) with agonistic antibodies against CD16, NKp46, 2B4 or NKG2D. After 24 hr, cells were washed and stained with LIVE/DEAD Fixable Yellow (ThermoFisher) followed by antibodies against CD56, CD16, CD69, PVRIG, TIGIT and DNAM-1. For analysis of short term kinetics, cells were incubated at 37° C. with the indicated stimuli, and at the specified timepoints were transferred to 4° C. Cells for the 0 timepoint were kept at 4° C. Upon completion of all timepoints, cells were stained with LIVE/DEAD Fixable Yellow followed by antibodies against CD56, CD16, CD69 and PVRIG.

### Other Methods

Details of experimental procedures (flow cytometry, Chromium release assay, degranulation assay) are provided below.

### Data Analysis

Flow cytometry data was analysed using FlowJo software (BD), and statistical analysis was performed in Prism (GraphPad). FIG. 4G was created with BioRender.com.

### Results

PVRIG Blockade Enhances NK Cell Killing of PVRL2^{hi}PVR^{lo} AML Cells

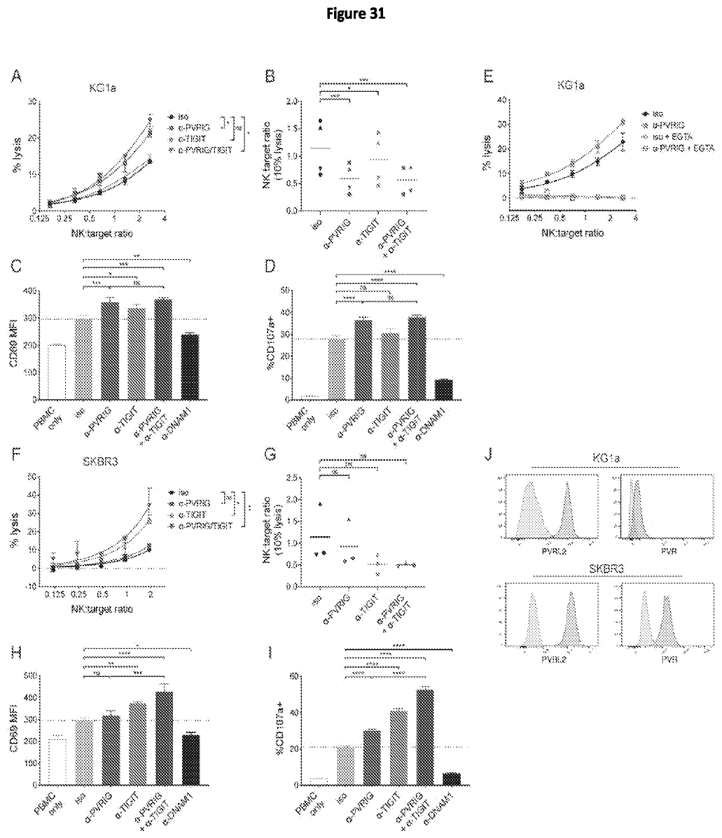

To assess whether PVRIG blockade could enhance NK cell responses against AML, we utilised the AML cell line KG1a. When co-cultured with healthy donor PBMCs and PVRIG blocking antibody, a significant increase in KG1a cell death was observed compared to the untreated control (FIG. 31A). In contrast, TIGIT blocking antibody did not significantly enhance KG1a target cell lysis, and KG1a lysis in the presence of combined anti-PVRIG and anti-TIGIT was comparable to PVRIG blockade alone (FIG. 31A). To compare across donors with variable baseline killing, we calculated the NK:target ratio required for 10% KG1a lysis for each donor. PVRIG blockade significantly decreased the NK:target ratio required to reach 10% KG1a lysis, while TIGIT blockade had only a minor effect (FIG. 31B). PVRIG blockade also significantly increased NK cell activation and degranulation, as measured by CD69 and CD107a staining respectively (FIG. 31C-D). TIGIT blockade had minimal effect on both NK cell activation and degranulation and combined PVRIG and TIGIT blockade showed no benefit over PVRIG blockade alone (FIG. 1C-D). The activating receptor DNAM-1 was important for recognition of targets, as blocking DNAM-1 significantly inhibited NK cell activation and degranulation (FIG. 31C-D, H-I). KG1a target cell death was perforin-dependent, as it was completely blocked when free calcium was complexed with EGTA (FIG. 31E).

PVRIG blockade was clearly more effective than TIGIT blockade for enhancing NK cell responses against KG1a but did not enhance lysis of the breast cancer cell line SKBR3. Rather, significantly more target cell death was observed with TIGIT blockade, or combined PVRIG and TIGIT blockade (FIG. 31F). Pooled data from 3 donors suggested TIGIT blockade, but not PVRIG blockade, decreased the NK:target ratio required for 10% lysis, but the difference did not reach statistical significance (FIG. 31G). TIGIT blockade significantly enhanced NK cell activation and degranulation, whereas PVRIG blockade had minimal effect on activation and a much smaller effect on degranulation (FIG. 31H-I). Combined PVRIG and TIGIT blockade enhanced NK cell activation and degranulation cytotoxicity even further, suggesting that PVRIG blockade can have an additive effect to TIGIT blockade (FIG. 31H-I).

NK cells from all healthy donors tested expressed both PVRIG and TIGIT (FIG. 39). Although the levels of expression (particularly of TIGIT) varied, all donors were consistently more responsive to PVRIG blockade against KG1a targets, and more responsive to TIGIT blockade against SKBR3 targets. Because TIGIT binds preferentially to PVR, while PVRIG binds exclusively to PVRL2, we explored whether differential expression of the ligands on KG1a and SKBR3 could explain the different NK cell responses to PVRIG or TIGIT blockade. Indeed, we observed that while both SKBR3 and KG1a cells have high expression of PVRL2, KG1a expressed far less PVR than SKBR3 (FIG. 31J). This suggests that tumours expressing high levels of PVRL2 but low levels of PVR are more likely to inhibit immune cells via PVRIG, whereas when both ligands are present, inhibition via TIGIT appears to predominate. This trend was also observed with other AML cell lines (FIG. 40). Both AML-193 and Kasumi-1 cells were PVRL2⁺PVR^{lo} (FIG. 40A) and were killed at significantly higher levels in the context of anti-PVRIG with PBMCs from healthy donors (FIG. 40B-C). By contrast, ML-2 and THP-1 AML cells were PVRL2⁺PVR⁺ (FIG. 40A) and were significantly increased killed in the presence of anti-TIGIT rather than anti-PVRIG (FIG. 40D-E).

AML Patient Bone Marrow Contains PVRL2^{hi}PVR^{lo} Blasts and PVRIG⁺ NK Cells

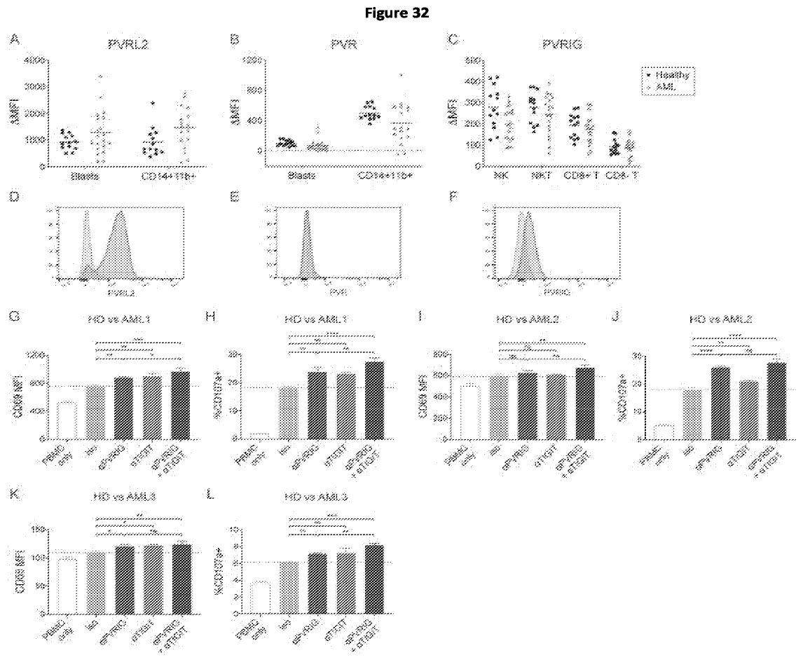

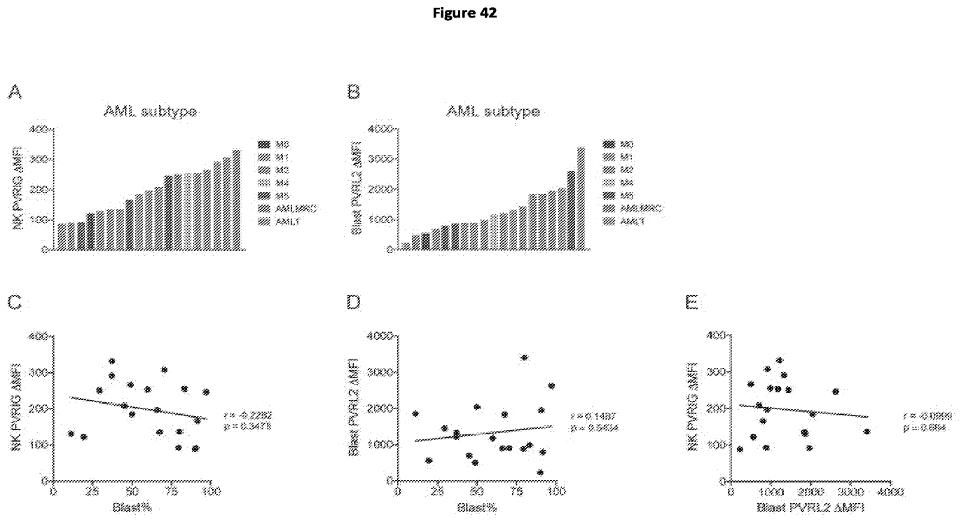

We next examined the expression of PVRIG, PVR and PVRL2 in AML patient bone marrow. Using multicolour flow cytometry, we distinguished various lymphoid (CD3⁻CD56⁺ NK cells, CD3⁺CD56⁺ NKT cells, CD3⁺CD8⁺ T cells, CD3⁺CD8⁻ T cells), myeloid (SSC^{hi}CD14⁺CD11b⁺ monocytes) and blast (CD45^{lo}SSC^{int}) populations collected at diagnosis (FIG. 41). Primary AML blasts were PVRL2⁺PVR^{lo}, and PVR was expressed at higher levels on CD14⁺CD11b⁺ monocytes (FIG. 32A-B, D-E). In healthy donor bone marrow samples, the CD45^{lo}SSC^{int} immature myeloid population (gated as per FIG. 41) were also PVRL2⁺PVR^{lo}, suggesting that high PVRL2 expression is a feature of normal myeloblasts (FIG. 32A-B). Nonetheless, the PVRL2^{hi}PVR^{lo} phenotype of the AML blasts suggested they would be a good target for PVRIG blockade, provided that patient effector cells expressed PVRIG. T cells and NK cells expressed PVRIG in all AML patients tested, with higher expression in NK, NKT and CD8⁺ T cells and lower expression in CD8⁻ T cells (FIG. 32C, F). There was no statistical difference in PVRIG expression levels on bone marrow immune subsets of AML patients compared with healthy donors (FIG. 32C). PVRIG and PVRL2 expression levels varied considerably amongst patients, but this did not correlate with AML subtype or the percentage of bone marrow blasts (FIG. 42).

To assess whether PVRIG blockade could enhance NK cell killing of AML patient blasts, we co-cultured healthy donor PBMCs with bone marrow from an AML patient with a high percentage (>90%) of PVRL2^{hi}PVR^{lo} AML blasts (identified in FIG. 2A-C by blue triangles). In this context, NK cells showed significantly increased CD69 expression and degranulation with PVRIG or TIGIT blockade. In addition, combined PVRIG and TIGIT blockade was associated with significantly higher NK cell activation and degranulation (FIG. 32G-H). Similar results were obtained for a further two AML pateints tested (FIG. 321-L). This indicates that PVRIG blockade or combination PVRIG/TIGIT blockade could enhance NK cell cytotoxicity against PVRL2⁺ tumour targets in AML patients.

### PVRIG Expression on NK Cells is Modulated by Activation.

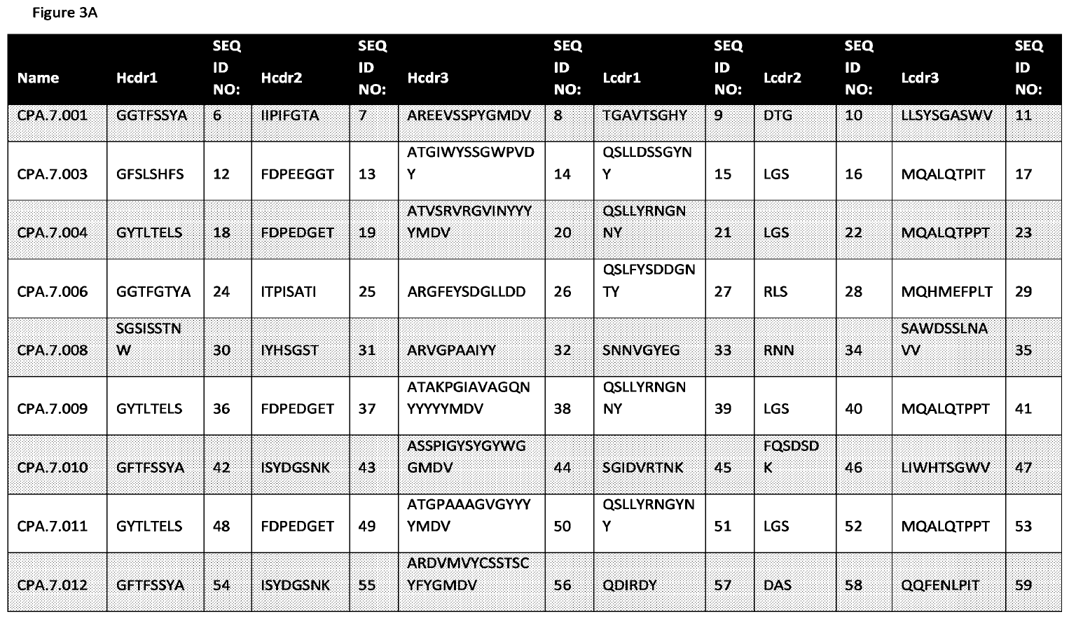

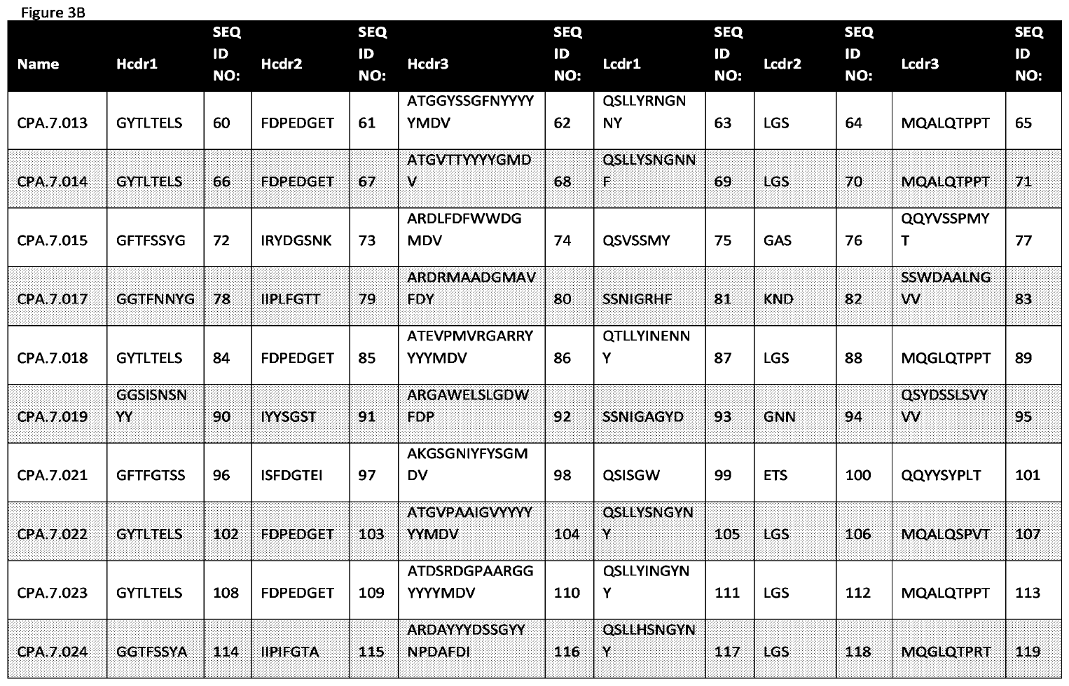

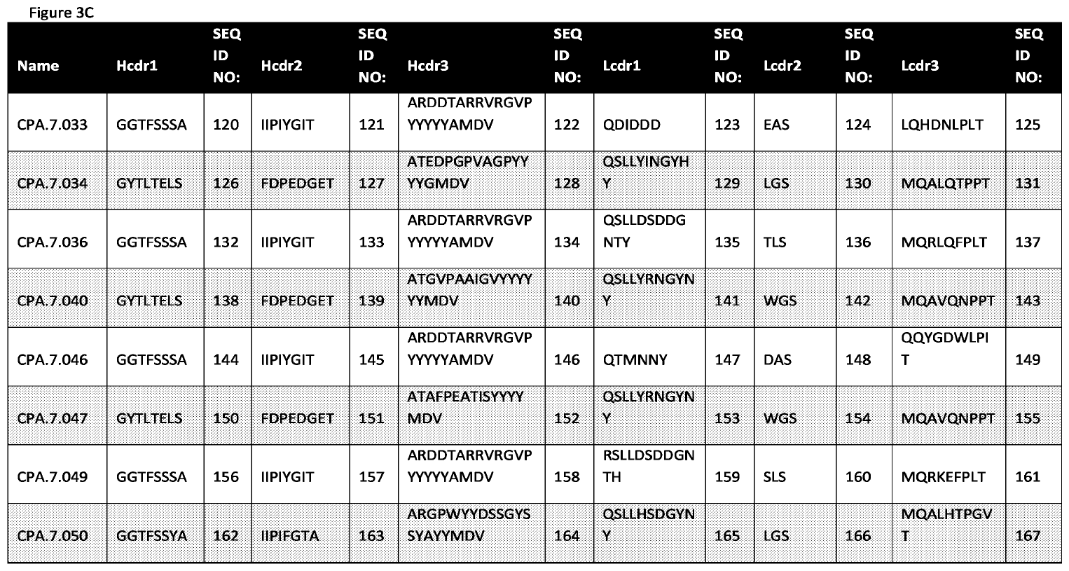

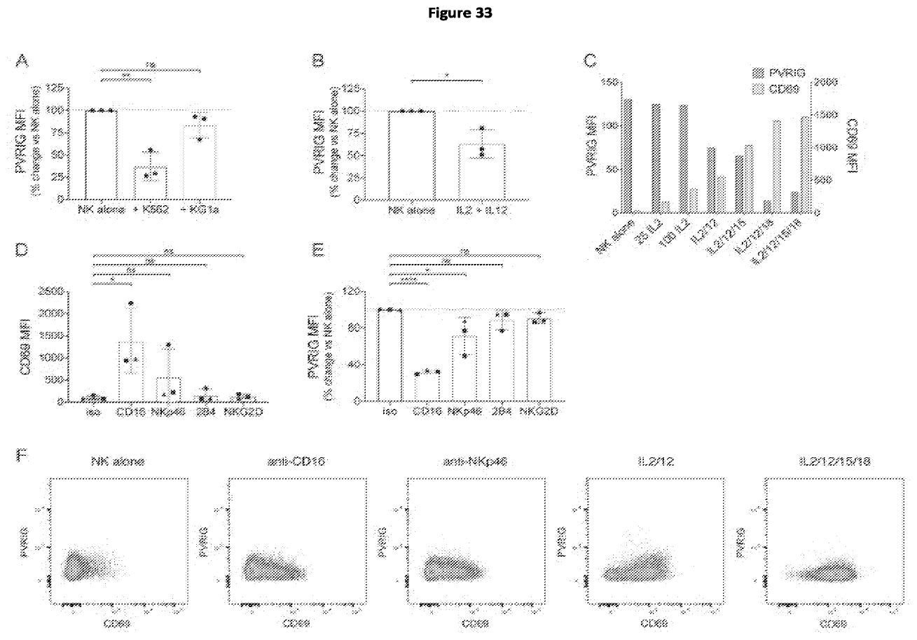

To understand why PVRIG was not upregulated on AML patient NK cells, we explored the mechanisms regulating NK cell PVRIG expression. To do this, we activated healthy donor NK cells for 24 hours via co-culture with tumour targets, NK activating cytokines or via agonistic antibodies to NK cell activating receptors. NK cells consistently decreased PVRIG expression after interaction with target cells, although to a much greater degree with K562 than KG1a cells (FIG. 33A). This may be because K562 lacks HLA class I, resulting in greater activation of the NK cells. We next investigated whether activation of NK cells via cytokines would also cause loss of PVRIG. Indeed, NK cells stimulated with IL-2 and IL-12 had significantly decreased PVRIG expression (FIG. 33B). Using IL-2 or combinations of IL-2, IL-12, IL-15 and IL-18, NK cells were increasingly activated, as measured by CD69 levels. Interestingly, NK cell surface PVRIG and CD69 levels were inversely correlated in NK cells undergoing cytokine-mediated activation (FIG. 33C). Stimulation of NK cells with plate-bound agonistic antibodies against the activating receptors CD16, NKp46, 2B4 and NKG2D also resulted in differing levels of activation (FIG. 33D) and a concomitant decrease in NK cell PVRIG levels (FIG. 33E). When PVRIG and CD69 expression was examined in individual NK cells, there was a trend for more activated NK cells (higher CD69) to express proportionally less PVRIG after anti-CD16 or anti-NKp46 stimulation (FIG. 3F). In contrast, following cytokine stimulation NK cells increased CD69 and decreased PVRIG expression more uniformly (FIG. 3F).

### TIGIT and DNAM-1 Expression on NK Cells is Modulated by Activation.

TIGIT and DNAM-1 are NK cell receptors within the same receptor-ligand axis as PVRIG.⁹⁻¹² We investigated whether a similar modulation of TIGIT and DNAM-1 occurred following NK cell activation. In contrast to PVRIG, TIGIT expression was increased after stimulation of NK cells with target cells, cytokines or agonistic antibodies (FIG. 34A-C), whereas changes in DNAM-1 levels were dependent on the stimulus. DNAM-1 was reduced by interaction with target cells (FIG. 34D), but increased after activation with IL-2 and IL-12 or anti-CD16 (FIG. 34E-F). Overall, our results indicated that the expression levels for different NK receptors are regulated differently, depending on the stimulus. PVRIG was consistently decreased and TIGIT increased upon NK cell activation, regardless of the stimulus, and the magnitude of change was correlated with the level of activation. On the other hand, DNAM-1 expression decreased upon target recognition, but was increased by activation via cytokines or agonistic antibodies to activating receptors (FIG. 34G). The loss of DNAM-1 may result from a form of immune evasion, which has previously been described to occur on contact with PVR⁺ tumour cells.²² DNAM-1 increase in response to stimulation via cytokines or activation receptors could be a means to increase the net activation signal, which, in conjunction with decreased PVRIG expression, could serve to lower the activation threshold of the NK cell.

Intracellular PVRIG does not Decrease Upon Activation

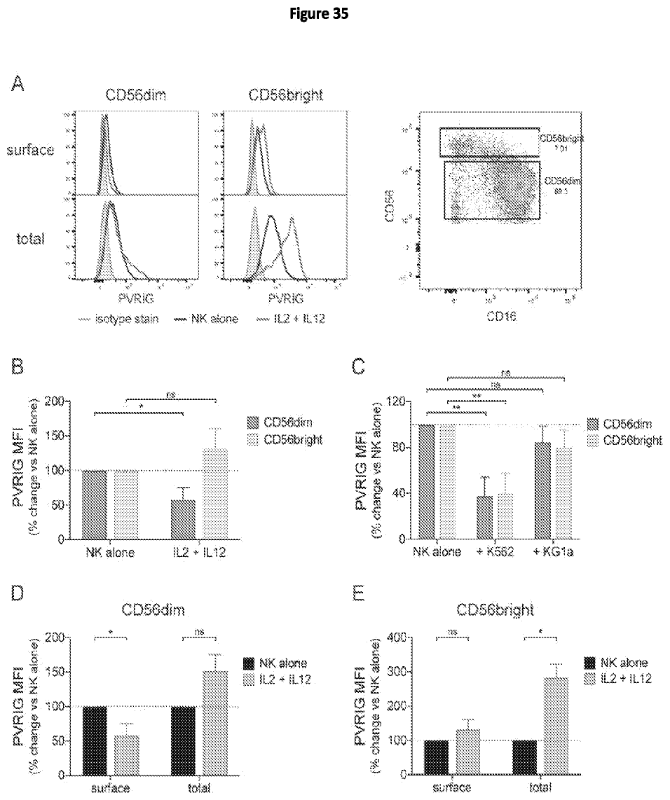

We next investigated whether PVRIG levels are regulated differently in the two principal peripheral blood NK cell subsets, CD56^{dim}CD16⁺ and CD56^{bright}CD16⁻. After IL-2 and IL-12 stimulation, the CD56^{dim} subset showed decreased PVRIG surface expression, as was seen previously for unfractionated NK cells (FIG. 35A-B). This was unsurprising given that CD56^{dim} comprise 90-95% of all circulating NK cells. By contrast, CD56^{bright} NK cells showed no loss of PVRIG (FIG. 35A-B). This was not due to failed activation, as both subsets upregulated CD69 equally (data not shown). Interestingly, this distinct pattern of regulation of PVRIG occurred only with cytokine stimulation (FIG. 35B), but not following interaction with target cells, which caused both subsets to downregulate PVRIG (FIG. 35C). We next determined whether an intracellular pool of PVRIG exists, or if it is solely expressed on the cell surface. Using permeabilised cells, we detected total (surface plus intracellular) PVRIG levels. For both CD56^{dim} and CD56^{bright} NK cell subsets, total PVRIG staining was far greater than surface staining, indicating that a pool of PVRIG is present in the cytosol (FIG. 35A). Interestingly, while CD56^{dim} NK cells lost their surface PVRIG upon activation with IL-2 and IL-12, the total amount of PVRIG was unchanged compared with untreated NK cells (FIG. 35D). This suggested that PVRIG lost from the cell surface upon activation was internalised. Alternatively, the total PVRIG level could be maintained despite decreased surface expression by synthesis of new molecules, which then egress to the cell surface. The latter appeared to be the case for CD56^{bright} NK cells, which maintained PVRIG surface expression and increased total PVRIG levels upon activation by IL-2 and IL-12 (FIG. 35E).

### NK Cell Surface PVRIG Levels are Maintained by Continuous Trafficking to the Cell Surface

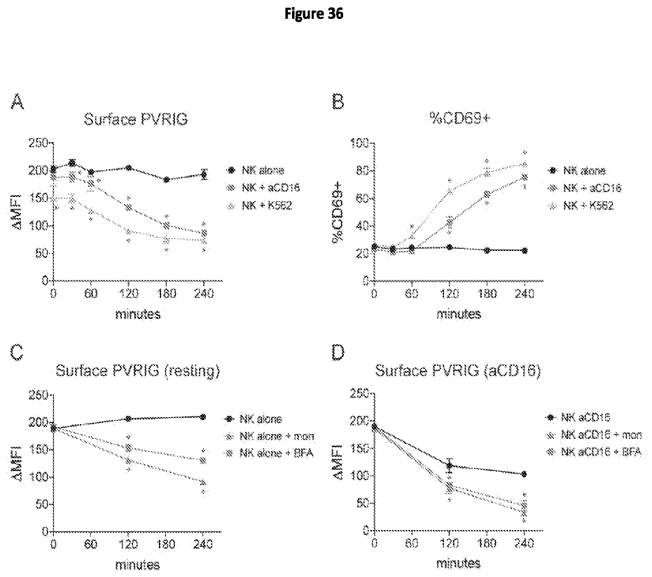

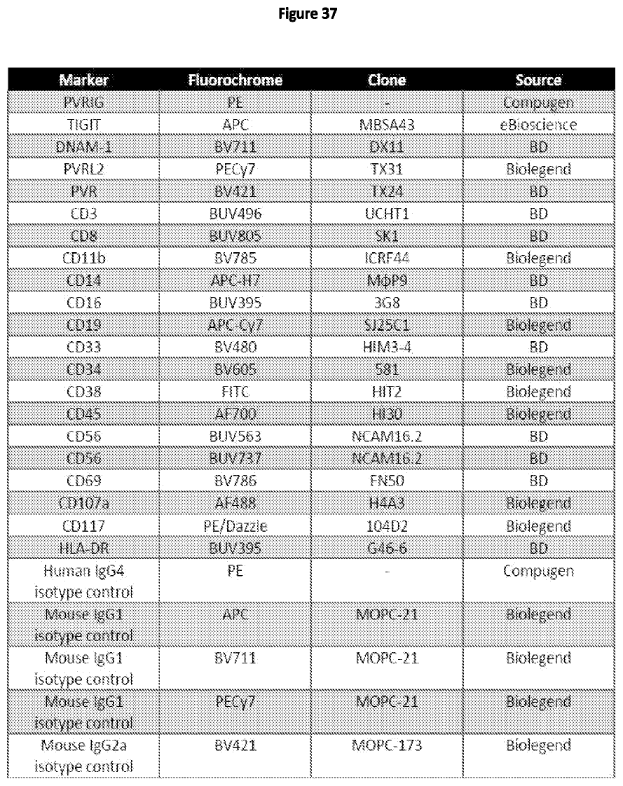

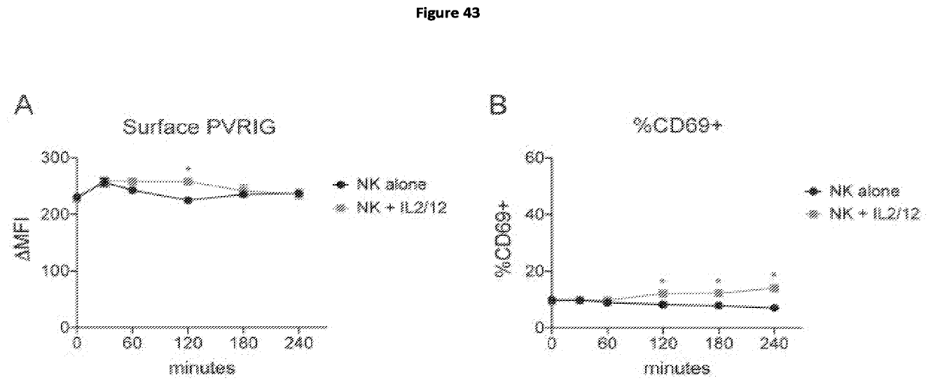

To further explore the mechanisms by which NK cells regulate surface PVRIG levels, we first assessed the kinetics of PVRIG loss from the cell surface under different stimulation conditions using a short term timecourse. NK cells co-cultured with K562 cells showed loss of PVRIG expression within 1-2 hours, at which time CD69 began to be upregulated (FIG. 36A-B). Stimulation of NK cells with anti-CD16 caused a similar level of PVRIG loss and activation, although K562 appeared to be the stronger stimulus at early time points (FIG. 36A-B). Within the 4 hours assessed, IL-2 and IL-12 stimulation had no appreciable effect on PVRIG expression, and minimal effect on activation (FIG. 43), indicating that cytokine stimulation influences PVRIG levels more slowly. Next, we used monensin and brefeldin A to determine whether intracellular trafficking via the ER and Golgi network was important for maintaining NK cell surface PVRIG. Both monensin and brefeldin A inhibit the trafficking of molecules to the cell surface, by disrupting the Golgi apparatus, trans-Golgi network or endosomal network.²³⁻²⁶ Untreated NK cells maintained constant PVRIG surface levels over 4 hours. However, the addition of either monensin or brefeldin A resulted in significant loss of surface PVRIG levels (FIG. 36C). Interestingly, the presence of monensin or brefeldin A also caused greater loss of PVRIG in NK cells undergoing activation via anti-CD16 stimulation (FIG. 36D). These results suggest there is trafficking of PVRIG molecules to the cell surface, via the ER and Golgi, in both untreated and activated NK cells (FIG. 36E).

In summary, PVRIG was downregulated on the NK cell surface following activation by tumour targets, anti-CD16 or cytokines. Furthermore, a pool of PVRIG is present in the NK cell cytoplasm, and cell surface PVRIG is maintained by trafficking to the surface. Taken together, our findings suggest that anti-PVRIG blocking antibodies enhanced NK cell killing of AML target cells by blocking PVRIG present on the NK cell surface. This resulted in decreased PVRL2-PVRIG mediated inhibition, and a decreased threshold for NK cell activation and increased AML blast killing.

### Discussion

Enhancing the activity of NK cells following HSCT may be beneficial for AML patients. NK cells are the first lymphoid cells to be reconstituted after HSCT, reaching normal levels within one month after transplant, much earlier than T cells.^{27, 28} However, their capacity to kill residual leukemic blasts can be limited by the interaction of NK inhibitory receptors with ligands in the tumour microenvironment.^{27, 29, 30} Thus, blocking inhibitory receptors such as PVRIG could potentially be useful after HSCT to enhance NK cell activity to delay or prevent relapse.

In this study, we showed PVRIG blockade enhanced human NK cell activity against PVRL2^{hi}PVR^{lo} AML target cells. AML blasts in patient bone marrow were PVRL2^{hi}PVR^{lo}, suggesting PVRIG blockade may increase NK-mediated killing of AML blasts. The AML blast PVRL2^{hi}PVR^{lo} phenotype is consistent with previous studies in AML patients.³¹ Our study is the first to report NK cell PVRIG expression in AML patient bone marrow. NK cell PVRIG expression was not upregulated in AML patients. Our subsequent analysis suggested PVRIG upregulation is not required for PVRIG blockade to be effective. Even though interaction with AML cells caused loss of PVRIG from the NK cell surface, PVRIG molecules trafficked via the ER-Golgi network and were then expressed on the cell surface. This suggests that, over time, a far greater amount of PVRIG is available on the cell surface to be blocked by anti-PVRIG antibodies than is detected at a single time point

In contrast to PVRIG, other NK cell immune checkpoint receptors, such as TIGIT, are upregulated with activation.^{2,11} Despite sharing the same ligand as TIGIT, the modulation of PVRIG does not follow this model, suggesting it could have a distinct biological function. It is possible that PVRIG acts as a regulator to keep NK cells in check in the steady state. The downregulation of PVRIG in response to cytokines or target recognition would then allow greater activation of NK cells in response to inflammatory stimuli. Our data showed NK cell PVRIG was present at higher levels in the cytoplasm than on the cell surface; this intracellular PVRIG was not decreased by activation. This cytoplasmic pool of PVRIG could represent either newly synthesized or recycled protein. A recent study by Whelan et al.³ examined PVRIG expression on isolated human T cells, and observed a similar trend for loss of PVRIG expression immediately after activation. However, sustained activation of T cells with antigen, IL-2 and IL-7 resulted in increased PVRIG expression by day 11.

This study reveals unique aspects of PVRIG biology that should be considered when determining potential indications for its therapeutic use. Our results suggest that PVRIG blockade may still have a therapeutic effect, provided the tumour cells express the PVRL2^{hi}PVR^{lo} phenotype. Furthermore, when AML blasts express both PVRL2 and PVR, combination PVRIG and TIGIT blockade may also induce an effective NK cell mediated anti-tumor response. These findings also have broader implications for the study of other checkpoint receptors. As more novel receptors are identified as potential targets, they should not be assumed to have the same biology as previously established immune checkpoints, and their potential efficacy should not necessarily be measured by the same parameters.

### Supplementary Methods

### Flow Cytometry Staining

In all experiments, cells were first labelled with LIVE/DEAD Fixable Yellow (ThermoFisher) in phosphate buffered saline (PBS) for 20 min at 4° C. and washed. For AML patient samples, cells were pre-incubated with human Fc block (BD Biosciences) for 10 min at 4° C. prior to surface staining. Cells were then stained with antibodies against surface markers in FACS buffer (PBS with 2% FCS) for 30 min at 4° C. Cells were then washed and fixed in 2% paraformaldehyde (PFA, ThermoFisher) for 15 min at 4° C., then resuspended in FACS buffer before acquisition on a FACSymphony (BD). For total (intracellular+surface) PVRIG staining, after staining for surface markers (CD56, CD16 and CD69) cells were washed and fixed using Fixation Buffer (eBioscience) for 20 min at 4° C., then washed and stained with anti-PVRIG-PE in Permeabilization Buffer (eBioscience) for 30 min at 4° C. Where indicated, DMFI of each marker was calculated as geometric mean of test—iso.

### Chromium Release Assay

Chromium release assay was performed as described previously.1 Briefly, target cells were labelled with 100 μCi Chromium-51 (51Cr, PerkinElmer) for 1 hr at 37° C., washed, then cocultured with PBMCs in triplicate wells at effector:target ratios from 32:1 to 2:1. Blocking or isotype control antibodies were each added at a final concentration of 10 μg/ml. EGTA (ethylene glycol-bis(2-aminoethylether)-N,N,N′,N′-tetraacetic acid, Sigma) was added at a final concentration of 4 mM. Wells with targets alone (spontaneous release) and targets with 10% Triton×100 (Sigma, maximum release) were included as controls. After 4 hr, supernatants were collected and the amount of 51Cr released detected using a gamma counter (Wallac Wizard). The % specific lysis was calculated by [(experimental release−spontaneous release)/(maximum release−spontaneous release)]*100. NK:target ratio was calculated from the percentage of NK cells found in PBMCs, determined by flow cytometry.

### Degranulation Assay

Target cells were labelled with Cell Trace Violet (ThermoFisher) in PBS for 10 min at 37° C., washed, then co-cultured with PBMCs in triplicate wells at the specified effector:target ratios. Blocking or isotype control antibodies were each added at a final concentration of 10 μg/ml, and anti-CD107a AF488 was included during the co-culture period. Wells with targets alone or PBMCs alone were included as controls. After 4 hr, cells were washed and stained with LIVE/DEAD Fixable Yellow followed by antibodies against CD56, CD16, CD3 and CD69. Due to variation in baseline CD69 levels between donors, CD69 MFI was normalised as a percentage of the isotype control treated group.

### REFERENCES

1. Zhu Y, Paniccia A, Schulick A C, et al. Identification of CD112R as a novel checkpoint for human T cells. J Exp Med. 2016; 213(2):167-176.
2. Xu F, Sunderland A, Zhou Y, Schulick R D, Edil B H, Zhu Y. Blockade of CD112R and TIGIT signaling sensitizes human natural killer cell functions. Cancer Immunol Immunother. 2017; 66(10):1367-1375.
3. Whelan S, Ophir E, Kotturi M F, et al. PVRIG and PVRL2 Are Induced in Cancer and Inhibit CD8(+) T-cell Function. Cancer Immunol Res. 2019; 7(2):257-268.
4. Li M, Qiao D, Pu J, Wang W, Zhu W, Liu H. Elevated Nectin-2 expression is involved in esophageal squamous cell carcinoma by promoting cell migration and invasion. Oncol Lett. 2018; 15(4):4731-4736.
5. Oshima T, Sato S, Kato J, et al. Nectin-2 is a potential target for antibody therapy of breast and ovarian cancers. Mol Cancer. 2013; 12(60.
6. Miao X, Yang Z L, Xiong L, et al. Nectin-2 and DDX3 are biomarkers for metastasis and poor prognosis of squamous cell/adenosquamous carcinomas and adenocarcinoma of gallbladder. Int J Clin Exp Pathol. 2013; 6(2):179-190.
7. Liang S, Yang Z, Li D, et al. The Clinical and Pathological Significance of Nectin-2 and DDX3 Expression in Pancreatic Ductal Adenocarcinomas. Dis Markers. 2015; 2015(379568.
8. Bekes I, Lob S, Holzheu I, et al. Nectin-2 in ovarian cancer: How is it expressed and what might be its functional role? Cancer Sci. 2019; 110(6):1872-1882.
9. Bottino C, Castriconi R, Pende D, et al. Identification of PVR (CD155) and Nectin-2 (CD112) as cell surface ligands for the human DNAM-1 (CD226) activating molecule. J Exp Med. 2003; 198(4):557-567.
10. Tahara-Hanaoka S, Shibuya K, Onoda Y, et al. Functional characterization of DNAM-1 (CD226) interaction with its ligands PVR (CD155) and nectin-2 (PRR-2/CD112). Int Immunol. 2004; 16(4):533-538.
11. Yu X, Harden K, Gonzalez L C, et al. The surface protein TIGIT suppresses T cell activation by promoting the generation of mature immunoregulatory dendritic cells. Nat Immunol. 2009; 10(1):48-57.
12. Stanietsky N, Simic H, Arapovic J, et al. The interaction of TIGIT with PVR and PVRL2 inhibits human NK cell cytotoxicity. Proc Natl Acad Sci USA. 2009; 106(42):17858-17863.
13. Deuss F A, Gully B S, Rossjohn J, Berry R. Recognition of nectin-2 by the natural killer cell receptor T cell immunoglobulin and ITIM domain (TIGIT). J Biol Chem. 2017; 292(27):11413-11422.
14. Murter B, Pan X, Ophir E, et al. Mouse PVRIG Has CD8(+) T Cell-Specific Coinhibitory Functions and Dampens Antitumor Immunity. Cancer Immunol Res. 2019; 7(2):244-256.
15. NCT03667716: COM701 in Subjects With Advanced Solid Tumors. [cited 22 Jan. 2020]; Available from: https://ClinicalTrials.gov/show/NCT03667716
16. Aptsiauri N, Ruiz-Cabello F, Garrido F. The transition from HLA-I positive to HLA-I negative primary tumors: the road to escape from T-cell responses. Curr Opin Immunol. 2018; 51(123-132.
17. Guillerey C, Smyth M J. Cancer Immunosurveillance by Natural Killer Cells and Other Innate Lymphoid Cells. In: Zitvogel L, Kroemer G, eds. Oncoimmunology: A Practical Guide for Cancer Immunotherapy. Cham: Springer International Publishing, 2018:163-180.
18. Ruggeri L, Capanni M, Casucci M, et al. Role of natural killer cell alloreactivity in HLA-mismatched hematopoietic stem cell transplantation. Blood. 1999; 94(1):333-339.
19. Ruggeri L, Capanni M, Urbani E, et al. Effectiveness of donor natural killer cell alloreactivity in mismatched hematopoietic transplants. Science. 2002; 295(5562):2097-2100.
20. Ruggeri L, Mancusi A, Capanni M, et al. Donor natural killer cell allorecognition of missing self in haploidentical hematopoietic transplantation for acute myeloid leukemia: challenging its predictive value. Blood. 2007; 110(1):433-440.
21. Howlader N, Noone A, Krapcho M, et al. SEER Cancer Statistics Review, 1975-2016, National Cancer Institute. Bethesda, Md. Available from https://seer.cancer.gov/csr/1975_2016/, based on November 2018 SEER data submission, posted to the SEER web site, April 2019.
22. Carlsten M, Norell H, Bryceson Y T, et al. Primary human tumor cells expressing CD155 impair tumor targeting by down-regulating DNAM-1 on NK cells. J Immunol. 2009; 183(8):4921-4930.
23. Mollenhauer H H, Morre D J, Rowe L D. Alteration of intracellular traffic by monensin; mechanism, specificity and relationship to toxicity. Biochim Biophys Acta. 1990; 1031(2):225-246.
24. Tartakoff A M. Perturbation of vesicular traffic with the carboxylic ionophore monensin. Cell. 1983; 32(4):1026-1028.
25. Fujiwara T, Oda K, Yokota S, Takatsuki A, Ikehara Y. Brefeldin A causes disassembly of the Golgi complex and accumulation of secretory proteins in the endoplasmic reticulum. J Biol Chem. 1988; 263(34):18545-18552.
26. Klausner R D, Donaldson J G, Lippincott-Schwartz J. Brefeldin A: insights into the control of membrane traffic and organelle structure. J Cell Biol. 1992; 116(5):1071-1080.
27. Nguyen S, Dhedin N, Vernant J P, et al. NK-cell reconstitution after haploidentical hematopoietic stem-cell transplantations: immaturity of NK cells and inhibitory effect of NKG2A override GvL effect. Blood. 2005; 105(10):4135-4142.
28. Mancusi A, Ruggeri L, Velardi A. Haploidentical hematopoietic transplantation for the cure of leukemia: from its biology to clinical translation. Blood. 2016; 128(23):2616-2623.
29. Muntasell A, Ochoa M C, Cordeiro L, et al. Targeting NK-cell checkpoints for cancer immunotherapy. Curr Opin Immunol. 2017; 45(73-81.
30. Carlsten M, Jaras M. Natural Killer Cells in Myeloid Malignancies: Immune Surveillance, NK Cell Dysfunction, and Pharmacological Opportunities to Bolster the Endogenous NK Cells. Front Immunol. 2019; 10(2357.
31. Sanchez-Correa B, Gayoso I, Bergua J M, et al. Decreased expression of DNAM-1 on NK cells from acute myeloid leukemia patients. Immunol Cell Biol. 2012; 90(1):109-115.
32. Hsu A K, Quach H, Tai T, et al. The immunostimulatory effect of lenalidomide on NK-cell function is profoundly inhibited by concurrent dexamethasone therapy. Blood. 2011; 117(5):1605-1613.
33. Arber D A, Orazi A, Hasserjian R, et al. The 2016 revision to the World Health Organization classification of myeloid neoplasms and acute leukemia. Blood. 2016; 127(20):2391-2405.

### Example 3: CPA.9.086.H4(S241P), A Novel Therapeutic Antibody Targeting Tigit Augments Anti-Tumor T Cell Function and the Activity of PVRIG and PD-1 Pathway Blockade

### Abstract

Immune checkpoint inhibitors (ICIs) have emerged as promising therapies for the treatment of cancer. However, existing ICIs, namely PD-(L)1 and CTLA-4 inhibitors, generate durable responses only in a subset of patients. TIGIT is a co-inhibitory receptor and member of the DNAM-1 family of immune modulating proteins. The prevalence of TIGIT and its cognate ligand, PVR (CD155), was evalutated in human cancers by assessing their expression in a large set of solid tumors. TIGIT is expressed on CD4⁺ and CD8⁺ TILs and is upregulated in tumors compared to normal tissues. PVR is expressed on tumor cells and tumor-associated macrophages from multiple solid tumors. The therapeutic potential of targeting TIGIT by generating CPA. 9.086.H4(S241P), a fully human anti-TIGIT hinge-stabilized IgG4 monoclonal antibody that binds specifically to human, cynomolgus monkey, and mouse TIGIT, and disrupts the binding of TIGIT with PVR was explored.

CPA.9.086.H4(S241P), either alone or in combination with a PVRIG (CHA.7.518.1.H4(S241P)) or PD-1 inhibitor, enhances antigen-specific human T cell responses in-vitro. In-vivo, a mouse chimeric version of CPA. 9.086.H4(S241P) in combination with an anti-PVRIG or anti-PD-L1 antibody inhibited tumor growth and increased survival in two syngeneic mouse tumor models. In summary, CPA.9.086.H4(S241P) enhances anti-tumor immune responses and is a promising candidate for the treatment of advanced malignancies.

### Introduction

Antibodies targeting checkpoint receptors such as CTLA-4 or PD-1 have revolutionized cancer treatment. However, most patients do not derive long-term benefit from cancer immunotherapies due to primary and adaptive resistance mechanisms, and one third of treated patients relapse by developing acquired resistance [1]. Therefore, targeting of additional immune-suppression mechanisms to overcome resistance to current immunotherapies may provide therapeutic benefit. Members of the DNAX accessory molecule-1 (DNAM-1) family that interact with nectin and nectin-like molecules recently emerged as important regulators of tumor immune surveillance. Members of the DNAM-1 axis are under preclinical and clinical investigation as targets for novel immunotherapies, including: TIGIT (T cell Ig and immunoreceptor tyrosine-based inhibitory motif [ITIM] domain), CD96, and PVRIG (poliovirus receptor related Ig domain containing protein). TIGIT and PVRIG are non-redundant inhibitory receptors within the same biological axis, with TIGIT being the high affinity, functional receptor for PVR, whereas PVRIG is the dominant functional receptor for PVRL2 (CD112) [2]. TIGIT is an inhibitory receptor on T and NK cells [3, 4]. TIGIT competes for PVR binding with the co-activatory receptors DNAM-1 (CD226) [4-7]. PVRIG was recently identified as a co-inhibitory functional receptor expressed on NK and T cells in the tumor microenvironment (TME) [2, 8]. PVRIG competes for PVRL2 binding with the co-activatory receptors DNAM-1. A Phase 1 clinical trial is currently underway to assess the safety and tolerability of a first-in-class therapeutic antibody targeting PVRIG (CHA.7.518.1.H4(S241P)) as a monotherapy and in combination with the PD-1 inhibitor, Nivolumab, in patients with advanced solid tumors [9].

Several studies have shown that TIGIT is a marker of T cell dysfunction and is upregulated on human viral-specific CD8⁺ T cells and tumor infiltrating T cells (TILs). In a murine model of chronic viral infection, TIGIT was found to be co-expressed with PD-1 on CD8⁺ T cells, and synergistic inhibition of TIGIT and PD-1 increased viral clearance and T cell effector function [10]. Similarly, in murine models of colon and breast carcinomas, anti-TIGIT and anti-PD-L1 antibodies treatment resulted in tumor rejection, an effect that was shown to be CD8⁺ T cell dependent [10]. In human settings, TIGIT blockade either alone or in combination with anti-PD-1 synergistically increased effector function of NY-ESO-1-specific CD8⁺ TILs isolated from melanoma patients [11]. Collectively, these data suggest that TIGIT and PD-1/PD-L1 co-blockade acts through CD8⁺ T cells to generate an effective anti-tumor immune response.

Currently, numerous antagonist TIGIT antibodies are in preclinical and clinical development to treat patients with locally advanced or metastatic tumors [12]. These early clinical studies have begun to shed light on whether TIGIT checkpoint inhibitors, either as a single agent or in combination with other cancer therapies, will generate durable responses in patients who do not benefit from anti-PD-1/PD-L1 therapies. Phase 1 studies to date have shown only limited responses for TIGIT inhibitors, either alone or in combination with anti-PD-1/PD-L1 antibodies [13-15]. However, the recent Phase 2 CITYSCAPE trial in non-small cell lung cancer (NSCLC) demonstrated a clear benefit of combining Tiragolumab with Atezolizumab relative to treatment with Atezolizumab alone [16]. Given the isotype differences between the antibodies in development [effector human IgG1 (hIgG1) versus non effector such as hIgG4 or Fc-silenced hIgG1], these studies may also determine whether isotype backbone influence the immune response developed upon anti-TIGIT therapy. All of the clinical data reported thus far has been with anti-TIGIT hIgG1 isotypes (Etigilimab, Tiragolumab, and Vibostolimab). CPA.9.086.H4(S241P) is a novel, fully human anti-TIGIT hinge stabilized IgG4 monoclonal antibody (mAb) that specifically binds TIGIT with high affinity and disrupts its interaction with the cognate ligand, PVR. TIGIT targeting combined with PVRIG or PD-1 pathway blockade represents a strategy for improving efficacy in patients that develop resistance or do not respond to PD-1/PD-L1 blockade alone.

### Materials and Methods

### Antibodies

CPA.9.086.H4(S241P) is a fully human anti-TIGIT IgG4 isolated by panning a phage display antibody library (XOMA Corp.) with human TIGIT (hTIGIT) ECD. CPA.9.086.H4(S241P) was optimized for improved affinity and cross-reactivity by saturation mutagenesis in the H-CDR2 and L-CDR3. CHA.7.518.1.H4(S241P) is a humanized anti-PVRIG IgG4 mAb and was described previously [2]. Pembrolizumab, anti-mPD-L1 mIgG1 (Clone YW243.55.S70), and isotype control antibodies were produced internally [17]. Chimeric CPA.9.086.H4(S241P) contains the human variable chains of CPA.9.086.H4(S241P) and the constant region of mIgG1. Anti-mouse PVRIG (mPVRIG) was described previously [18].

### Tumor Processing and Flow Cytometry

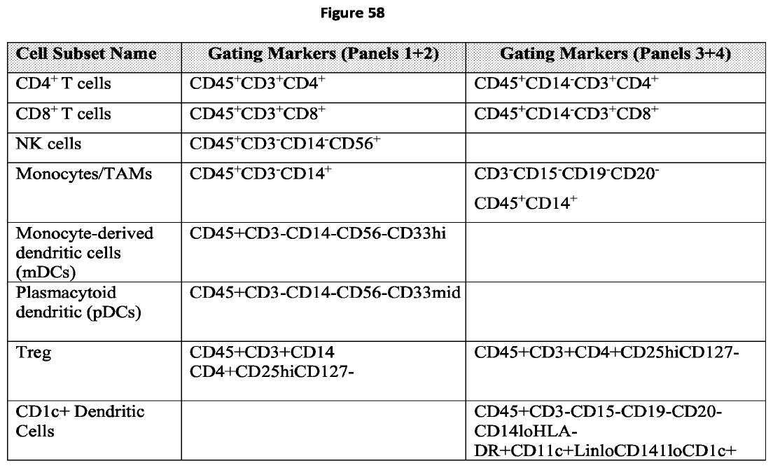

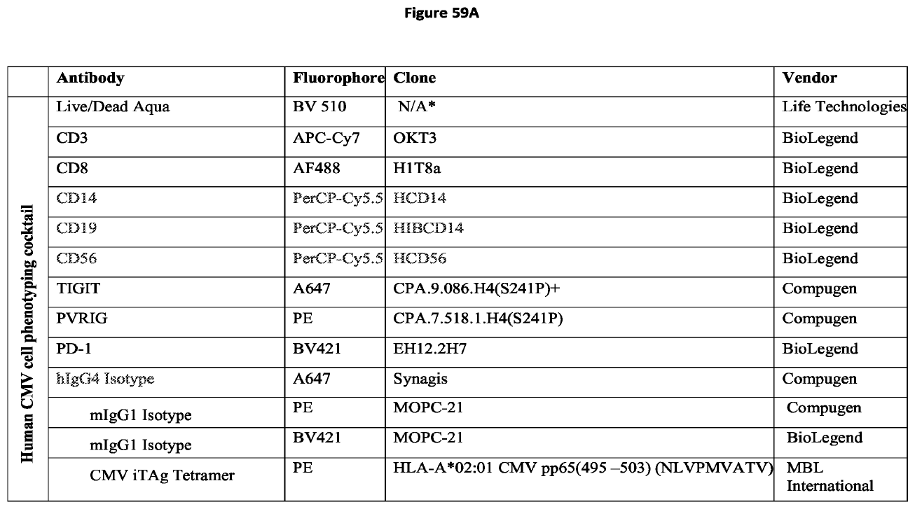

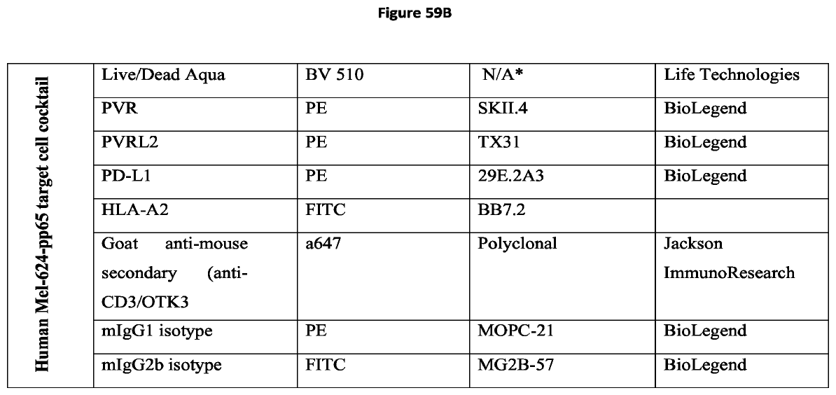

Human tumor samples were acquired from the Cooperative Human Tissue Network (CHTN). The tumor and histological type were determined from the pathology report (FIG. 56). Lineage marker and isotypes control antibodies are presented in FIG. 57. Samples were acquired on a Fortessa X-20 cytometer (BD Biosciences). Data was analyzed using FlowJo (TreeStar LLC) with the gating lineages defined in FIG. 58.

### KinExA

Kinetic exclusion assay (KinExA 3200 instrument, Sapidyne Instruments) was used to measure hTIGIT (Sino Biologicals) binding to CPA.9.086.H4(S241P). Six total binding curves were acquired with [hTIGIT]=854 aM-588 pM equilibrated for ˜72 hours with [CPA.9.086.H4(S241P)]_{binding sites}=1.1 pM-5.7 pM and ˜24 hours with [CPA.9.086.H4(S241P)]_{binding sites}=28.7 pM at 22° C. All six curves were simultaneously fit to a 1:1 equilibrium model using the KinExA software to estimate K_{D}. Detection beads were hTIGIT coupled to Ultralink Support resin (Thermo Scientific) and the secondary detection antibody was AF647-labeled goat anti-human IgG, Fc-fragment specific (Jackson ImmunoResearch Laboratories, West Grove, Pa.).

### Immunohistochemistry

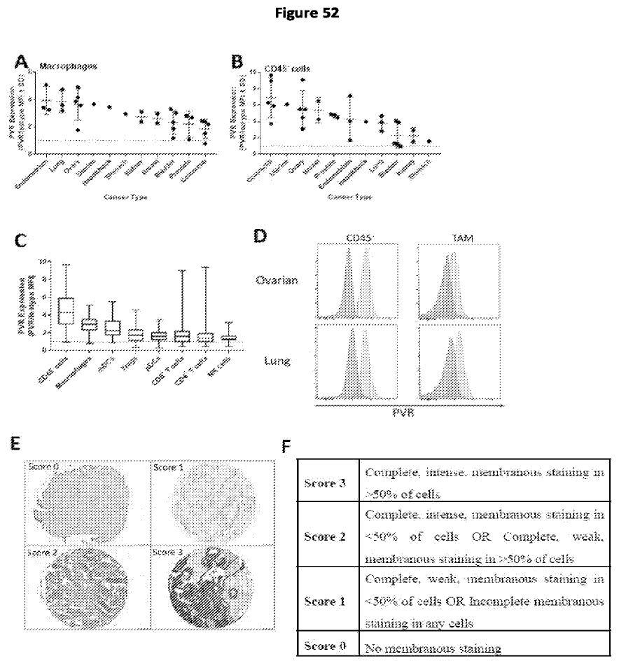

Expression of hPVR in human formalin fixed paraffin embedded (FFPE) tissues was evaluated using an anti-hPVR rabbit mAb (Clone D8A5G; Cell Signaling Technologies. Immuno-histochemistry (IHC) staining was conducted using an IntelliPATH™ automated staining platform (Biocare). Human PVR expression in FFPE TMA samples was scored by a pathologist on a quantitative scale based on the percentage of positive cells, and the completeness and intensity of the cell membrane staining (FIG. 52F). Halo software (Indica labs) was used for digital image analysis (OracleBio, Scotland, UK).

### TIGIT Binding and Blocking Assays

ExPi293 cells were engineered to express hTIGIT, cTIGIT, or mTIGIT. For binding experiments, cells were incubated for 1 hour with CPA.9.086.H4(S241P) or hIgG4 isotype control and detected with a secondary antibody. For blocking experiments, the cell lines described above were pre-incubated with CPA. 9.086.H4(S241P) or hIgG4 isotype for 30 minutes. Recombinant hPVR-mIgG2a-Fc (hPVR-Fc) and cynomolgus PVR-hIgG1-Fc-biotin (cPVR-Fc) produced internally, or mouse PVR-hIgG1-Fc (Sino Biological) were added to the cells for 1 hour and detected with labeled fluorescently labeled secondary antibodies. The fluorescent signal was detected by flow cytometry and corresponded to the % of PVR blocking to cell-surface expressed TIGIT.

### CDC and ADCC Assays

For complement dependent cytotoxicity (CDC), 5% baby rabbit serum containing complement (Cedarlane) was added to activated human CD8+ T cells. CPA.9.086.H4(S241P), hIgG4 isotype, Campath (anti-human CD52 hIgG1), or hIgG1 isotype were added and cell lysis was measured using a Cytotox-Glo reagent kit (Promega). For ADCC assays, activated human CD8⁺ T cells were labeled with DELFIA BATDA reagent (Perkin Elmer) and combined with activated NK cells. The same antibodies as used for the CDC assay were added. Time resolved fluorescence (TRF) signal was detected after 4 hours of incubation using an EnVision multi-label reader (Perkin Elmer).

### Jurkat Reporter Assay

Jurkat cells were engineered to express hTIGIT and a luciferase reporter driven by an IL-2 response element (Jurkat hTIGIT-IL2) (Promega, TIGIT/CD155 Blockade Bioassay). Jurkat hTIGIT-IL2 cells were cultured with CHO-K1 cells engineered to express human CD155 (hPVR) and a TCR activating complex (CHO-K1 hPVR-TCR). CPA.9.086.H4(S241P) or hIgG4 isotype were added to the co-culture and luminesce was quantified on an EnVision multi-label reader (Perkin Elmer).

### CMV CD8⁺ T Cell Co-Culture Assay

CMV antigen-specific T cells were prepared as described previously [2] and co-cultured for 18 hours with Mel-624 (ATCC) cells that were engineered to over-express the CMV peptide pp65₍₄₉₅₋₅₀₃₎. A human IFN-γ flex cytokine bead assay kit (BD Biosciences) was used to detect secreted IFN-γ in the cell culture supernatant. For T cell mediated cell killing, Mel-624 cells were engineered to over-express human PVR, PVRL2, and firefly luciferase (Mel-624-PVR-PVRL2-luc). Mel-624 cells were pulsed with pp65₍₄₉₅₋₅₀₃₎ peptide for 1 hour and combined with CMV-specific CD8⁺ cells. Bio-Glo luciferase substrate (Promega) was added after 16 hours according to the manufacturer's instructions. Luminesce was detected using a EnVision multi-label reader (Perkin Elmer).

### NK Cell Cytotoxicity Assay

Purified human NK cells were cultured for 16 hours with recombinant human IL-15 (rh-IL-15, R&D systems) then added to a co-culture with CAL-27 (ATCC) cells at a 10:1 E:T ratio for 4 hours in the presence of CHA.7.518.1.H4(S241P), CPA.9.086.H4(S241P) (10 μg/mL each), CHA.7.518.1.H4(S241P)+CPA.9.086.H4(S241P), or a hIgG4 isotype control. NK cell cytotoxicity was measured by the expression of CD107a on NK cells.

### OVA-Specific Mouse CD8⁺ T Cell Co-Culture Assay

Ovabumin (OVA)-specific CD8⁺ T cells were generated from the spleens of female C57BL/6-Tg (TcraTcrb)1100Mjb/J mice (OT-1, The Jackson Laboratory). Splenocytes were stimulated for 48 hours with 1 μg/mL H-2K^{b}-restricted OVA peptide (SIINFEKL, Anaspec) and rhIL-2. MC38 cells were pulsed with OT-1 peptide and combined with OVA-specific CD8+ T cells. Chimeric CPA.9.086.H4(S241P), anti-mouse PD-L1, mIgG1 or rat IgG2b (rIgG2b) isotype controls were added and plates were incubated for 24 hours. Mouse IFN-γ was measured using a mouse IFN-γ Flex Set Kit (BD Biosciences).

### Syngeneic Mouse Tumor Models

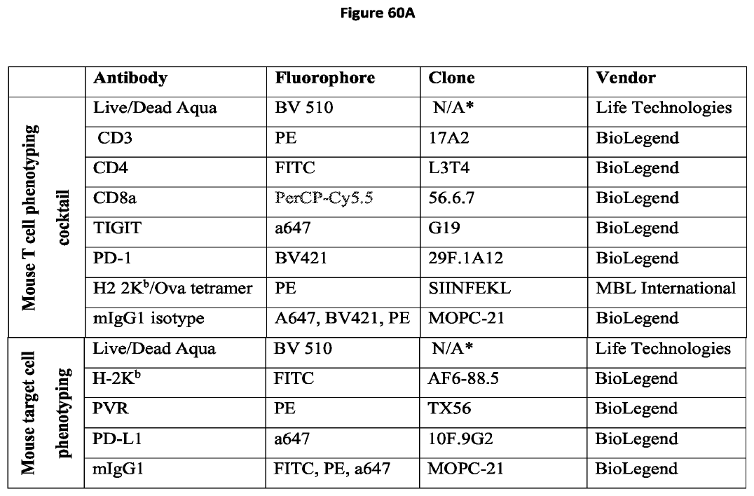

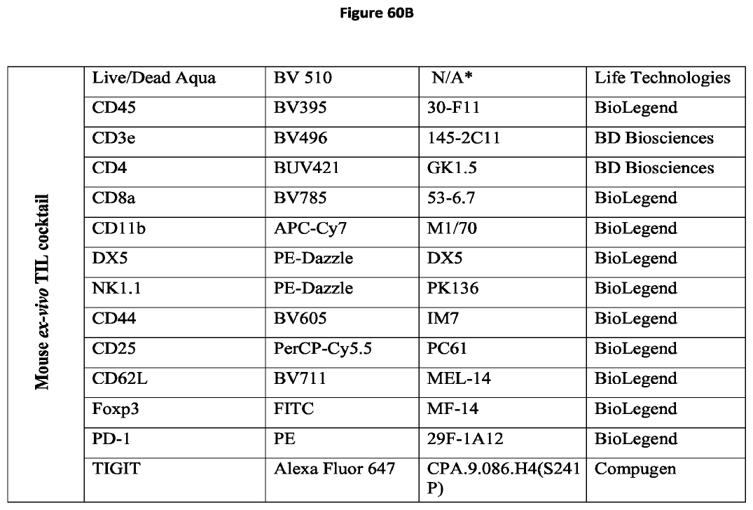

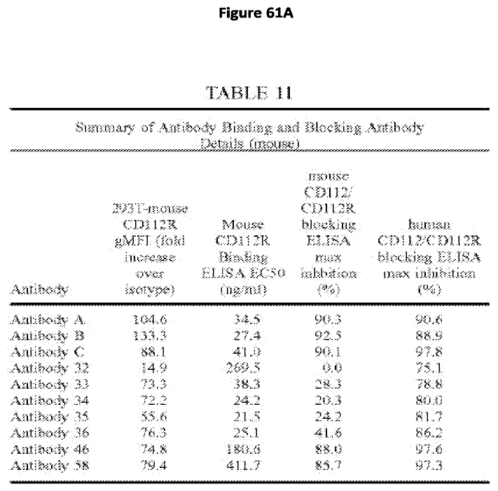

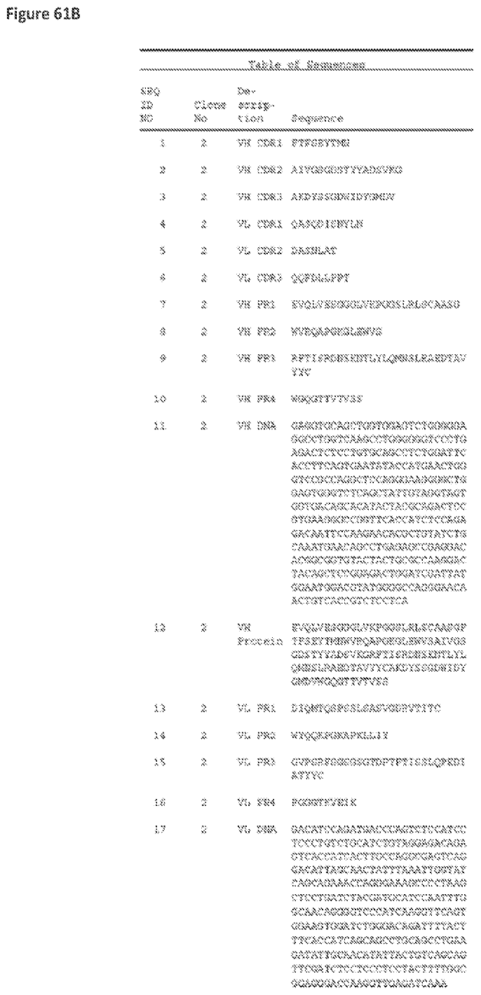

5×10⁵ CT26 cells or 1×10⁶ Renca cells (ATCC) were inoculated subcutaneously into the right flank of female Balb/c mice. Mice with tumors measuring 20-55 mm³ were randomized (into groups of 10 mice). Antibodies were administered through intraperitoneal injection on day 6 (monotherapy) or day 8 (combination therapy) post tumor inoculation and dosed three times a week for two weeks. Chimeric CPA.9.086.H4(S241P) mIgG1, anti-mPVRIG mIgG1 or mIgG1 isotype were administered at a dose of 10 mg/kg. Anti-mPD-L1 was administered at a dose of 3 mg/kg. Mice were euthanized when the tumor volumes reached 2000 mm³. To isolate TILs, CT26 tumors were dissociated with GentleMACs™ kits (Miltenyi Biotec). Lineage marker and isotypes control antibodies used are presented in FIG. 60. Gating lineages used are described in FIG. 60. All animals were housed during the study in an internal animal facility with food and water provided, ad libitum. All studies were approved by the Institutional Animal Care and Use Committee at the Compugen USA, Inc.

### Statistics and Calculations

The “log (agonist) vs. response—variable slope (four parameters)” model in Prism was used to model dose-response data from in vitro functional assays. Data were analyzed by paired Student t test; *, P<0.05; **, P<0.01; ***, P<0.001. Estimates of the half maximal inhibitory concentration (IC₅₀) or half maximal effective concentration (EC₅₀) were calculated based on these models. The percentage of specific cytotoxicity was calculated as: (1−(RLU_{(target cells+ T cells+antibody)}/RLU_{(target cells+ T cells+media alone)}))×100. For in-vivo studies, two-way ANOVA with repeated measures for selected pairs of groups was performed using JUMP software (Statistical Discoveries™). Tumor growth inhibition (TGI) at termination was performed by comparing tumor volumes measured on the last day on which all study animals were alive. Statistical differences in survival was determined by Log-rank P-value associated with each set of Kaplan-Meier curves.

### Results

### TIGIT is Expressed and Induced in Solid Tumors

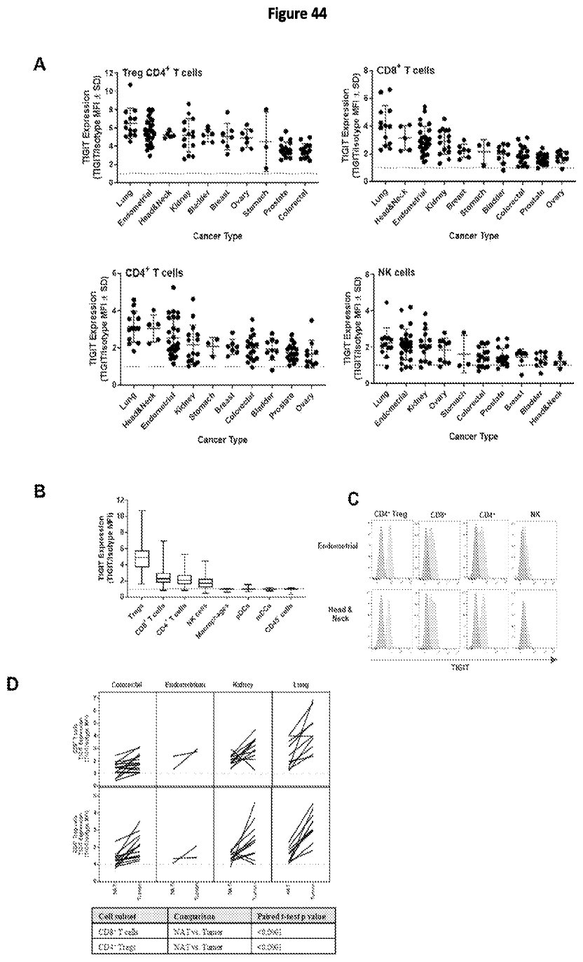

Although TIGIT protein expression has been examined on TILs in small subsets of human tumors [10, 11, 19, 20], expression across a large number of cancers with multiple tumors per indication has not been investigated. Thus, to further examine TIGIT in the TME, we assessed expression in ˜130 unique dissociated human tumors from 10 different cancer indications. TIGIT expression was detected on Tregs, CD8⁺ T cells, non-Treg CD4⁺ T cells, and NK cells from multiple tumor types (FIG. 44A). Across all tumors, TIGIT was expressed, from highest to lowest, on Tregs, CD8⁺ T cells, non-Treg CD4⁺ T cells, and NK cells (FIG. 1B-C). TIGIT expression was higher on CD4⁺ Treg and CD8⁺ T cells derived from tumor tissue compared to matched NAT (FIG. 44D). No TIGIT expression was detected on monocytes, DCs, or non-immune cells (FIG. 44B). Across all examined indications, lung, endometrium, head and neck and kidney cancers had the highest TIGIT expression for all lymphocyte subsets evaluated (FIG. 44A). As CD8⁺ T and NK cells are known to be important cytotoxic lymphocytes within the immune system, high TIGIT expression on these cell types suggests that TIGIT plays a critical role in regulating their activity.

### PVR is Expressed in Multiple Tumor Types

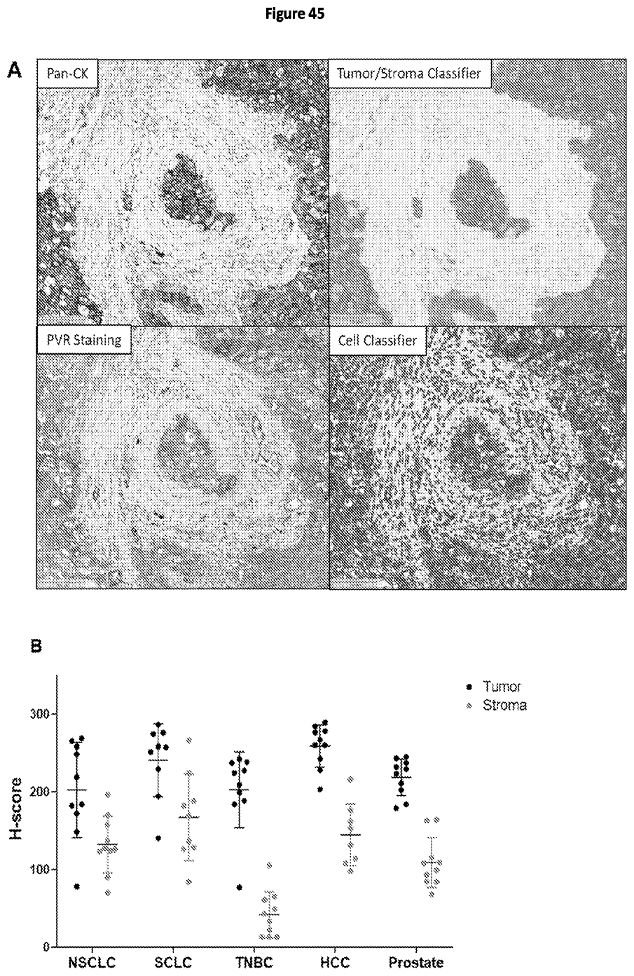

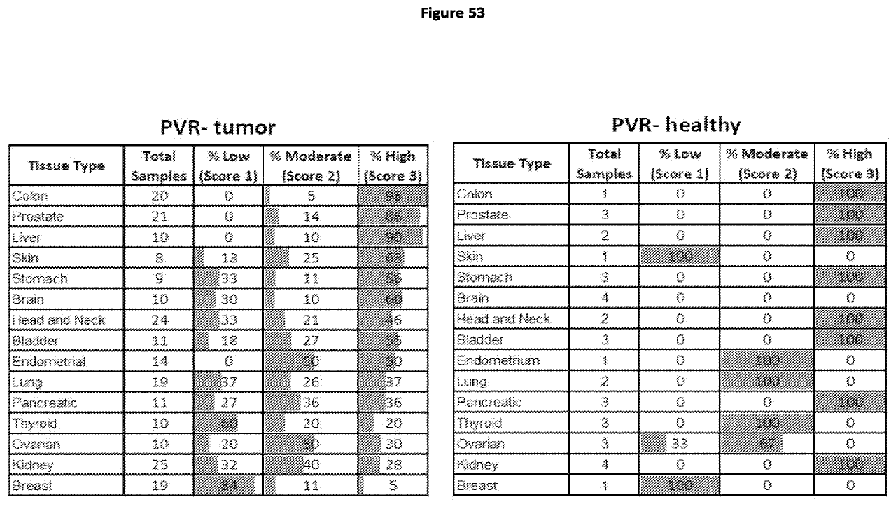

A subset of samples that were evaluated for TIGIT expression were also examined by flow cytometry for PVR expression. PVR expression was detected on two major cell subsets: 1) myeloid cells, and 2) non-immune CD45⁻ cells, (FIG. 52A-D). Significant correlations between the prevalence of TIGIT versus PVR expression could not be generated due to sample size limitations. PVR expression was also evaluated in FFPE tissues by IHC. Ten non-small cell lung cancer (NSCLC), small-cell lung cancer (SCLC), triple negative breast cancer (TNBC), hepatocellular cancer (HCC), and prostate cancer tumors were stained with anti-hPVR and pan-cytokeratin (Pan-CK). Using a novel digital image analysis for scoring PVR expression, the tumor/stromal border, cell segmentation, and membrane versus cytoplasmic staining delineation were determined (FIG. 45A). All five tumor types had high PVR expression, with H-scores ranging from 80-280 (FIG. 45B). Across all tumor types examined, PVR expression was higher in the tumor compartment compared to the stromal area (FIG. 2B). As PVR expression was highest on malignant cells, we further evaluated PVR expression on a panel of tissue microarrays (TMAs) from 15 tumor types. PVR expression was scored using a 0, 1, 2, or 3 scoring system (FIGS. 52E and 52F). PVR was detected in multiple tumor types, with the highest expression in colon cancer samples (FIG. 53).

### CPA.9.086.H4(S241P) Binds to TIGIT and Blocks TIGIT:PVR Interactions

The binding affinity of CPA.9.086.H4(S241P) to TIGIT was characterized by both a cell-based antibody binding assay and kinetic exclusion (KinExA).

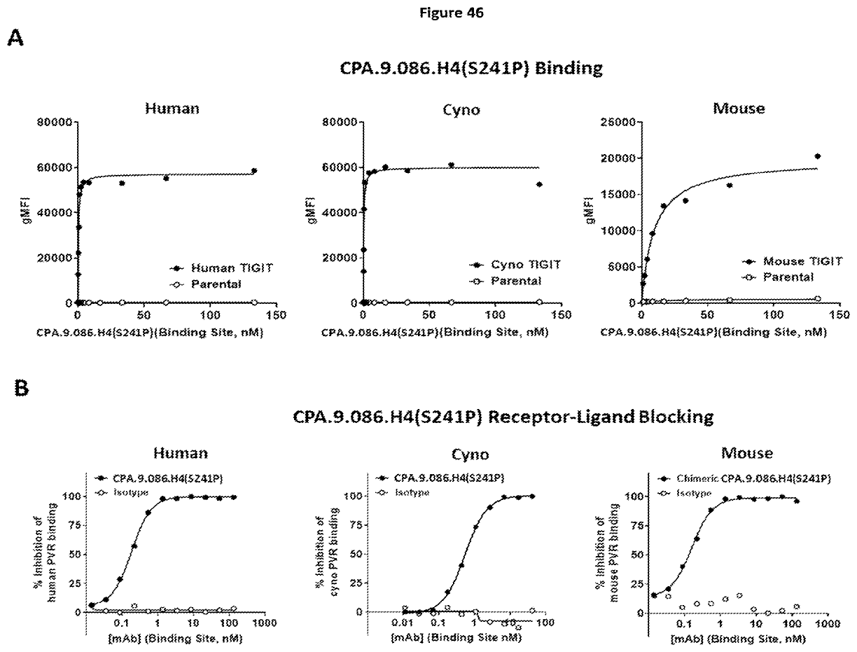

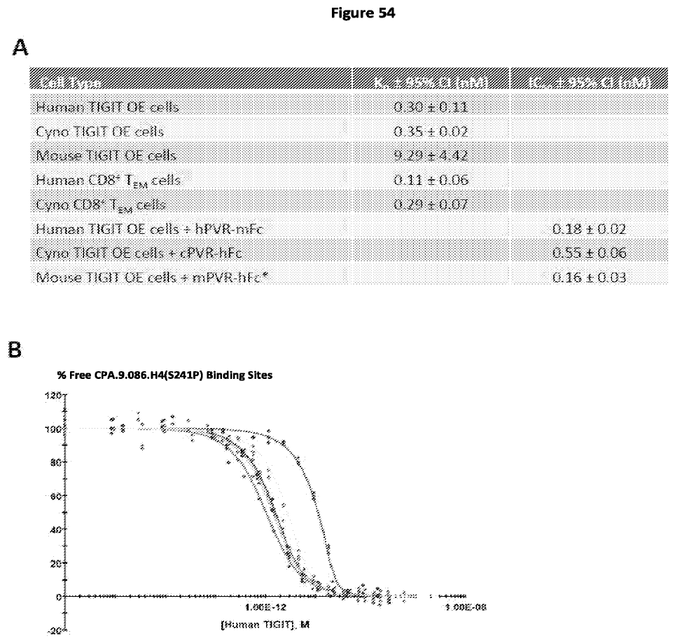

CPA.9.086.H4(S241P) bound with similar high affinity to hTIGIT and cTIGIT expressed on cells, while much lower binding affinity was observed for mTIGIT (FIG. 46A, FIG. 54A). Binding of CPA.9.086.H4(S241P) to hTIGIT expressed on the cell surface yielded K_{D} values ranging from 0.11-0.3 nM. Binding of CPA.9.086.H4(S241P) to a monomeric recombinant hTIGIT protein using KinExA yielded a K_{D} value of 626 fM (FIG. 54B).

Given the low affinity interaction and questionable biologic relevance of TIGIT binding to PVRL2 and PVRL3 [2-4], we focused on the ability of CPA.9.086.H4(S241P) to block the interaction of TIGIT with its high-affinity ligand, PVR. We utilized a cell-based blocking assay in which TIGIT was expressed on the cell surface and PVR was added as a recombinant protein. CPA.9.086.H4(S241P) showed complete inhibition of the human and cynomolgus TIGIT:PVR interaction, with IC₅₀'s of 0.18±0.02 nM and 0.55±0.03 nM, respectively (FIG. 46B, FIG. 54A). Additionally, we assessed ability of a chimeric CPA.9.086.H4(S241P) mIgG1 to block the mouse TIGIT:PVR interaction. The chimeric antibody blocked the interaction of mPVR-Fc to mTIGIT with an IC₅₀ of 0.16±0.03 nM. Taken together, these data suggest that CPA.9.086.H4(S241P) shares a similar mechanism of action across human, cynomolgus monkey and mouse.

### CPA.9.086.H4(S241P) Does Not Demonstrate ADCC or CDC Activity

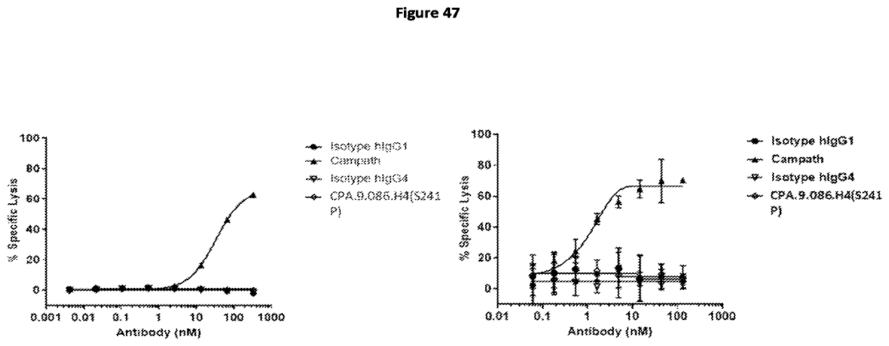

TIGIT is highly expressed on CD8⁺ TILs which are important mediators of the anti-tumor immune response [21]. Since depletion of TIGIT⁺ lymphocytes would be an undesirable outcome of CPA.9.086.H4(S241P) on-target binding, we assessed whether CPA.9.086.H4(S241P) could mediate Fc-dependent CDC or ADCC against TIGIT⁺ T cells. For CDC assessment, activated TIGIT⁺ human T cells were incubated with complement-containing baby rabbit serum. CPA.9.086.H4(S241P), or anti-CD52 (Campath) was added to the culture, and the amount of cell lysis was determined. CPA.9.086.H4(S241P) did not mediate CDC whereas Campath mediated CDC in a dose-dependent manner (FIG. 47A). To assess ADCC activity, activated TIGIT⁺ human T cells were co-cultured with activated human NKs. In contrast to Campath, which induced ADCC in a dose dependent manner, CPA.9.086.H4(S241P) did not demonstrate ADCC activity (FIG. 47B). CPA.9.086.H4(S241P) Enhances Human Lymphocyte Function In-Vitro

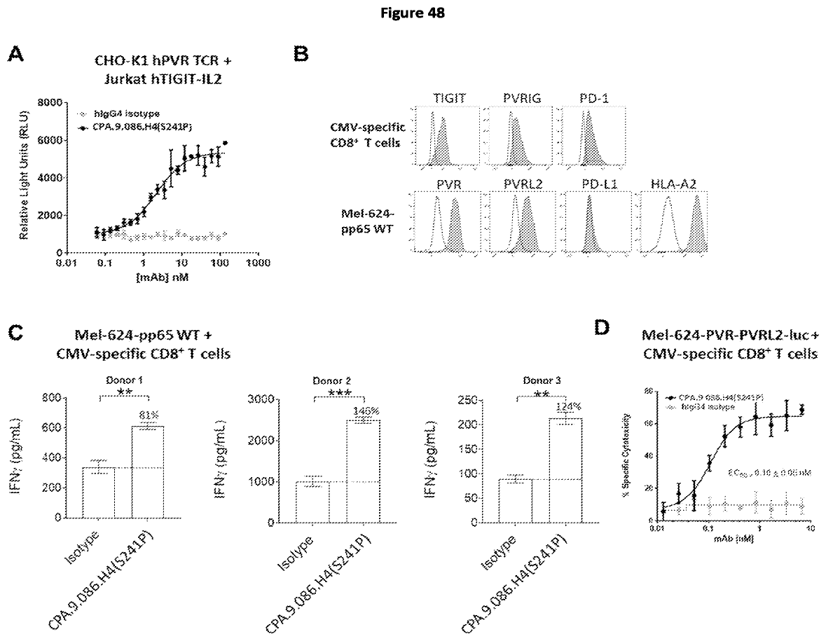

To evaluate the functional effect of TIGIT:PVR interaction blockade by CPA.9.086.H4(S241P), we utilized a Jurkat luciferase reporter co-culture system. CHO-K1 cells expressing anti-CD3 (OKT3) and hPVR (CHO-K1-OKT3-PVR⁺) were incubated with Jurkat-TIGIT-IL2-luc reporter cells. As shown in FIG. 48A, blockade of TIGIT:PVR by CPA.9.086.H4(S241P) increased luciferase production, with an EC₅₀ of 2.14 nM. Next, we evaluated the effect of CPA.9.086.H4(S241P) on antigen-specific T cell function using a peptide re-stimulation assay. PBMCs were activated with a viral pp65₍₄₉₅₋₅₀₃₎ antigen peptide and CMV-reactive effector CD8⁺ T cells were assessed for TIGIT, PVRIG, and PD-1 expression (FIG. 48B). CMV-specific CD8+ T cells were co-cultured with a PVR⁺ cancer cell line ectopically expressing the pp65 protein (Mel-624-pp65) (FIG. 48B). CPA.9.086.H4(S241P) significantly increased IFN-γ secretion for all three donors tested (FIG. 48C). Since a primary function of effector CD8+ T cells in the TME is cytotoxic cell killing, we modified the re-stimulation assay to evaluate this readout. Mel-624 cells engineered to ectopically express firefly luciferase, were pulsed with pp65(495-503) peptide and co-cultured with CMV-specific CD8⁺ T cells. CPA.9.086.H4(S241P) increased the specific cell killing of Mel-624 in a dose-dependent manner, with an EC₅₀ of 0.01±0.05 nM (FIG. 48D).

CPA.9.086.H4(S241P) Combined with PVRIG or PD-1 Pathway Blockade Enhances Human Lymphocyte Function

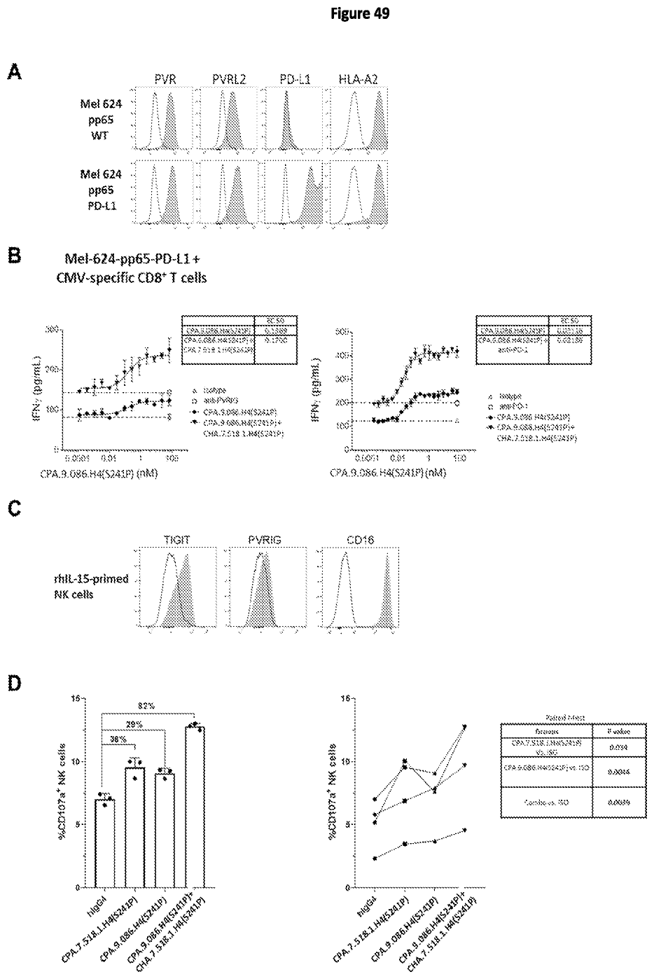

To assess CPA.9.086.H4(S241P) in combination with PVRIG or PD-1 blockade, CMV-reactive CD8⁺ T cells were co-cultured with Mel-624-pp65 cells and CPA.9.086.H4(S241P) in the presence or absence of anti-PVRIG, CHA.7.518.1.H4(S241P) antibody (FIG. 49A). CPA.9.086.H4(S241P) combined with CHA.7.518.1.H4(S241P) significantly increased IFN-γ secretion. Since Mel-624 cells express low endogenous PD-L1, we over-expressed hPD-L1 on cell surface (Mel-624-pp65 PD-L1+, FIG. 49A) and co-cultured them with CMV-reactive CD8⁺ T cells. Similar to the synergistic effects induced by combining CPA.9.086.H4(S241P) and CHA.7.518.1.H4(S241P), CPA.9.086.H4(S241P) combined with an anti-PD-1 mAb significantly increased IFN-γ secretion compared to CPA.9.086.H4(S241P) or anti-PD-1 alone in all tested donors (FIG. 49B, right panel). The average±SEM EC₅₀ value for CPA.9.086.H4(S241P) in the presence of anti-PD-1 was 0.03 nM±0.01 nM. The effects of combining TIGIT and PVRIG blockade on NK cell cytotoxic function were also evaluated. Priming of NK cells with hrIL-15 induced the expression of CD16 and TIGIT and to a lesser extent of PVRIG (FIG. 49C). While monotherapy with CPA.9.086.H4(S241P) or CHA.7.518.1.H4(S241P) augmented NK cell activity by 36% and 29%, respectively, as measured by CD107a expression, combination of both antibodies yielded a synergistic effect, enhancing NK cell cytotoxicity by 82% (FIG. 49D, left panel). These effects were maintained across four distinct healthy donors and were statistically significant (FIG. 49D, right panel). Collectively, these data show that blockade of TIGIT:PVR interaction by CPA.9.086.H4(S241P) enhances lymphocyte cytokine secretion and cytotoxicity. Furthermore, this effect can be magnified by combining CPA.9.086.H4(S241P) with blockade of other immune checkpoints, such as PVRIG and PD-1.

Chimeric CPA.9.086.H4(S241P) Combined with Anti-PVRIG or Anti-PD-L1 Induces Tumor Growth Inhibition

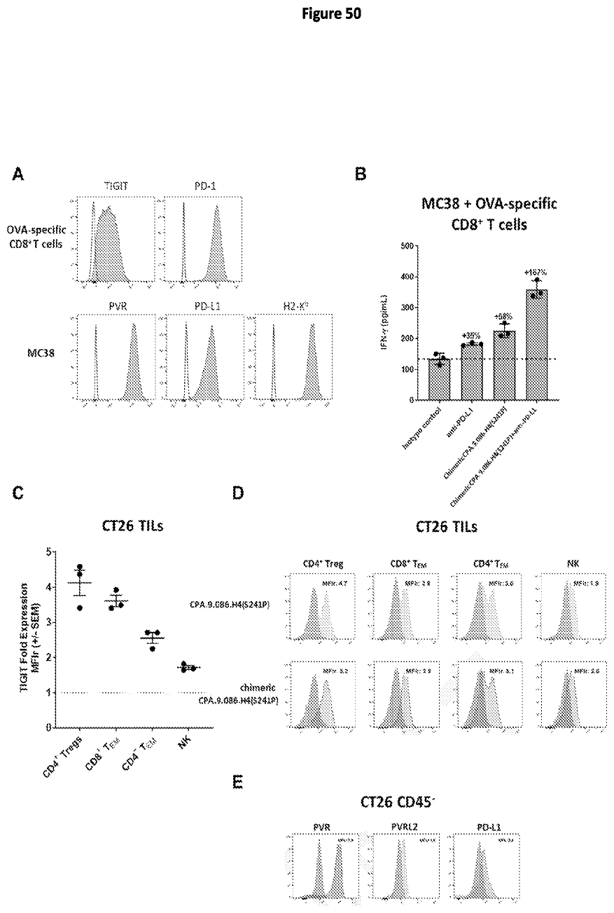

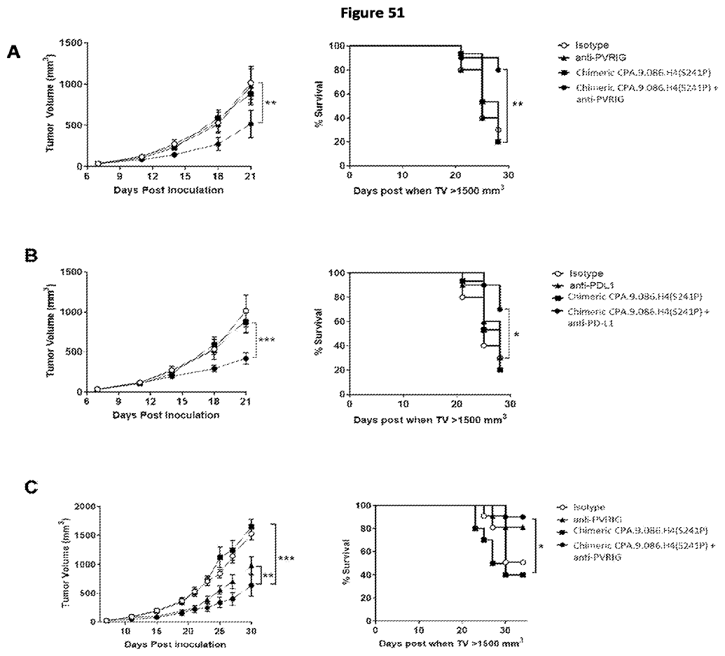

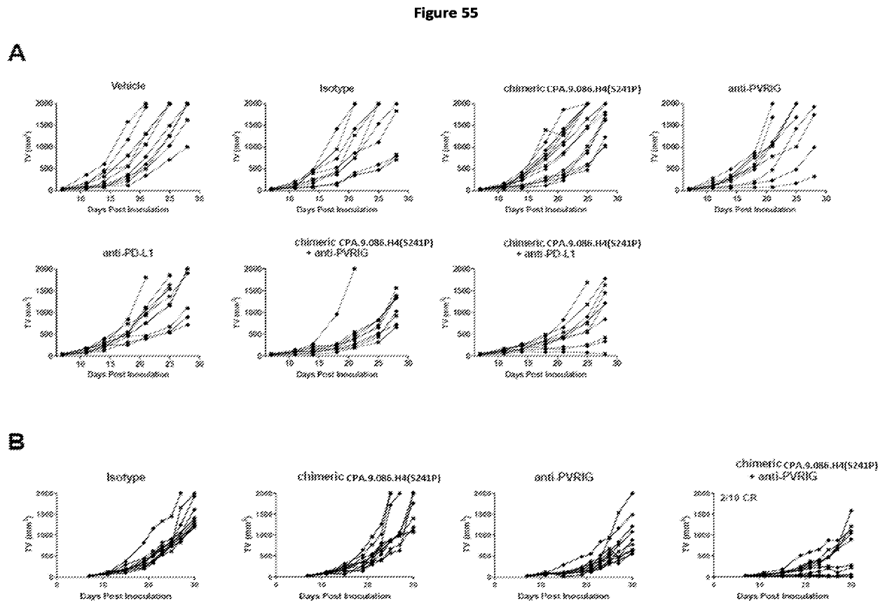

To determine if chimeric CPA.9.086.H4(S241P) maintained similar functionality compared to CPA.9.086.H4(S241P), we used a murine CD8⁺ T cell re-stimulation assay system. Activated OVA-specific CD8⁺ T cells were combined with a mPVR⁺ H-2K^{b+} cancer cell line (MC38) that had been pulsed with the OVA₍₂₅₇₋₂₆₄₎ peptide. Expression of TIGIT and PD-1 on OVA-specific CD8+ T cells and PVR, PD-L1, and H2-K^{b} on MC38 cells is shown in FIG. 50A. Treatment with chimeric CPA.9.086.H4(S241P) or anti-PD-L1 antibody alone led to enhanced IFN-γ secretion while the combination further increased IFN-γ release to 167% above the isotype control (FIG. 50B). To determine whether the observed effects of chimeric CPA.9.086.H4(S241P) in-vitro translate in-vivo, we assessed the effect of CPA.9.086.H4(S241P) on the colon carcinoma CT26 tumor model. Similar to the expression pattern we observed in dissociated human tumors (FIG. 44), mouse TIGIT was expressed on both T and NK lymphocytes isolated from CT26 tumors, with the highest expression found on CD4⁺ Tregs followed by CD8⁺ TEM cells and NK cells (FIG. 50C-D). Additionally, PVR, PVRL2 and PD-L1 were abundantly expressed on the CD45⁻ cell population within CT26 tumors (FIG. 50E). Tumor bearing mice treated with the chimeric CPA.9.086.H4(S241P) mIgG1 antibody as a single agent showed similar tumor growth and survival rates as compared to control groups (FIG. 51A-B, FIG. 55). The combination of chimeric CPA.9.086.H4(S241P) and anti-mPVRIG treatment resulted in a significant TGI and increase in overall survival compared to anti-PVRIG monotherapy (FIG. 51A). Similarly, combination of chimeric CPA.9.086.H4(S241P) and anti-PD-L1 resulted in significant TGI and enhanced overall survival compared to treatment with anti-PD-L1 monotherapy (FIG. 51B, FIG. 55). We also evaluated the combination of chimeric CPA.9.086.H4(S241P) and anti-mPVRIG using the Renca renal carcinoma tumor model. This model was selected since TIGIT, PVRIG, PVR and PVRL2 are expressed in Renca-tumor bearing mice (data not shown). The combination resulted in a significant TGI and an enhanced overall survival (FIG. 51C, FIG. 55). Collectively, these results demonstrate the potential of CPA.9.086.H4(S241P) in combination with anti-PVRIG or anti-PD-L1 to enhance anti-tumor lymphocyte responses and inhibit tumor growth in-vivo.

### Discussion

The TME shapes the T cell dysfunctional state through a diverse stimuli, including TCR triggering in the absence of co-stimulation, chronic antigen exposure [22-24] and an altered milieu of secreted and metabolic factors [25]. TILs often display alterations in TCR signaling pathways, express variety of inhibitory receptors and fail to produce effector molecules. TIGIT is a member of the immunomodulatory DNAM-1 axis that includes numerous immune receptors and ligands. Previous studies have demonstrated that TIGIT expression is highly associated with T cell exhaustion and marks activated, antigen-experienced, and dysfunctional T cells [10, 11, 26]. Correlation of TIGIT with PD-1 expression in human malignancies suggests that blockade of TIGIT binding to its cognate ligand, PVR, could synergize with anti-PD-1 cancer treatment [10]. Recently, we identified and described the PVRIG pathway as a non-redundant negative signaling node within the DNAM axis that synergizes with TIGIT and PD-1 inhibitors [2]. Taken together, these findings support investigating the clinical rationale of combining TIGIT blockade with PVRIG and PD-1 inhibitors.

To evaluate TIGIT and PVR expression within the TME of human tumors, we utilized multi-color flow cytometry and IHC. TIGIT was expressed on CD4⁺, CD8⁺, and NK cells isolated from tumors, with the highest expression on CD4⁺ regulatory T-cells, which was consistent with other reports analyzing protein and single cell RNA data from various tumor indications [19, 26, 27]. Importantly, compared to matched NAT lymphocytes, TIGIT was markedly upregulated on TILs indicative of their exhausted state. We determined that PVR was expressed on CD45⁻ tumor and stroma cells as well as CD14⁺ myeloid cells (likely composed of tumor associated macrophages). The presence of elevated levels of TIGIT on immune cells and abundant PVR expression across multiple tumor types on tumor cells and CD14⁺ TAMs indicates that this signaling pathway could be a pivotal mechanism for tumor evasion and provides the rationale for investigating effects of TIGIT blockade on immune modulation. In support of this hypothesis, previous studies have shown that PVR overexpression is closely correlated with enhanced tumor progression and poor clinical outcomes [20, 28, 29].

Next, to assess the therapeutic potential of TIGIT blockade for the treatment of solid and hematological malignancies, we developed CPA.9.086.H4(S241P), a fully human hinge stabilized IgG4 monoclonal antibody, that is specific for TIGIT and blocks its binding to human, cynomolgus, and mouse PVR. We selected an IgG4 isotype to minimize unintended Fc/receptor-mediated cytotoxicity against TIGIT⁺ CD8⁺ TILs. As confirmed experimentally in CDC and ADCC assays, CPA. 9.086.H4(S241P) is unlikely to elicit direct cytotoxicity of TIGIT⁺ cells. The exceptionally high affinity and long off-rate of CPA.9.086.H4(S241P) binding to human TIGIT, 626 fM and 2 days, respectively as measured by Kinexa, could also offer significant clinical advantages. Due to the fact that lower clearance rates have been reported for antibodies with high affinity [30], CPA.9.086.H4(S241P) may have a more significant biological effect at lower doses could be amenable to more frequent dosing compared to other TIGIT antibodies with lower binding affinities.

To study the effects of CPA.9.086.H4(S241P) on human immune cell function, we carried out three in-vitro assays: a Jurkat reporter assay, an antigen-specific CD8⁺ T cell co-culture assay, and a NK cell cytotoxicity assay. We found that CPA.9.086.H4(S241P) increased IL-2 signaling from Jurkat cells in a dose-dependent manner, suggesting that blockade of TIGIT and PVR binding by CPA.9.086.H4(S241P) can enhance pro-inflammatory cytokine signaling. Prior studies have established that the properties of dysfunctional exhausted T cells are shared between cancer and chronic infection [25]. In particular, the expression of inhibitory receptors such as TIGIT, PVRIG, and PD-1 is upregulated on both CMV-specific effector cells and on TILs. Thus, our in-vitro assay system that utilizes viral specific CD8⁺ T cells is relevant for modeling T cell responses at the tumor and immune system interface.

The addition of CPA.9.086.H4(S241P) to the co-cultures of CMV-specific CD8⁺ T cell and Mel-624 cells significantly increased IFN-γ secretion and target cell specific cytotoxicity. We and others have shown co-expression of PD-1, TIGIT and PVRIG on TILs [2, 10, 31, 32]. Accordingly, blocking TIGIT with CPA.9.086.H4(S241P) combined with either PVRIG or PD-1 pathway blockade significantly induced IFN-γ secretion compared to blockade of each individual pathway alone. Furthermore, the presence of a fixed dose of anti-PVRIG or anti-PD-1 did not significantly change the EC₅₀'s for CPA.9.086.H4(S241P), confirming that combining the inhibition of these non-redundant pathways should not alter the potency of CPA.9.086.H4(S241P). Finally, we demonstrated that the co-blockade of TIGIT and PVRIG not only enhanced CD8⁺ T cell function, but also increased human NK cell cytotoxicity.

To determine if CPA.9.086.H4(S241P) could also enhance the anti-tumor immune response in-vivo, we designed a chimeric CPA.9.086.H4(S241P) with mIgG1 Fc, to maintain a minimal degree Fcγ receptor binding, similar to the hIgG4 antibody. Consistent with data generated with CPA.9.086.H4(S241P) in human assay systems, chimeric CPA.9.086.H4(S241P), both alone and in combination with anti-mouse PD-L1, increased in-vitro immune function of OVA-specific mouse CD8⁺ T cells. To evaluate the effect of chimeric CPA.9.086.H4(S241P) in-vivo, we selected the CT26 and Renca syngeneic tumor models. Although monotherapy with chimeric CPA.9.086.H4(S241P) did not show efficacy in either model, the combination of chimeric CPA.9.086.H4(S241P) with anti-PD-L1 or anti-PVRIG both produced significant TGI and increased survival. While other studies have shown anti-tumor activity of TIGIT blockade with effector mIgG2a backbone in combination with anti-PD-L1 in syngeneic mouse tumor models [10], here we show significant TGI with a non-effector function anti-TIGIT mIgG1 antibody combined with a PD-L1 or PVRIG inhibitor. In addition, although studies with anti-TIGIT mIgG2a did demonstrate monotherapy activity in-vivo, mouse models do not mirror the structural diversity and expression pattern of the human Fc receptor systems [33] and thus their translational relevance for human isotype selection is questionable, as exemplified by early clinical results with hIgG1 isotype anti-TIGIT antibodies.

As we and others have shown, TIGIT is expressed on CD4⁺ Tregs, which suppress tumor immunity, as well as CD8⁺ effector cells that mediate the anti-tumor immunity (FIG. 44). Given this overlap in TIGIT expression, an effector function TIGIT antibody, which has the potential to deplete Tregs, also carries the risk of depleting CD8⁺ TILs. For this reason, TIGIT antibodies with hIgG4 or hIgG1 effector silenced isotypes, such as CPA.9.086.H4(S241P), would not deplete CD8⁺ TILs and therefore may have an advantage over TIGIT antibodies with wild type hIgG1 backbones. Collectively, this study highlights the functional activity of CPA.9.086.H4(S241P) and provides a strong rationale for combining CPA.9.086.H4(S241P) with PD-1 or PVRIG blockade for the rejuvenation of the anti-tumor immune response and effective eradication of human malignancies. Additional studies investigating the mechanism of action of TIGIT blockade as well as translational studies from ongoing clinical trials will be important to help further understand the factors that determine the biological effect and clinical efficacy of TIGIT inhibitors.

### REFERENCES FOR EXAMPLE 3

1. Sharma P, Hu-Lieskovan S, Wargo J A, Ribas A (2017) Primary, Adaptive, and Acquired Resistance to Cancer Immunotherapy. Cell 168:707-723. https://doi.org/10.1016/j.cell.2017.01.017
2. Whelan S, Ophir E, Kotturi M F, et al (2019) PVRIG and PVRL2 are induced in cancer and inhibit CD8+ T-cell function. Cancer Immunol Res 7:257-268. https://doi.org/10.1158/2326-6066.CIR-18-0442
3. Stanietsky N, Simic H, Arapovic J, et al (2009) The interaction of TIGIT with PVR and PVRL2 inhibits human NK cell cytotoxicity. Proc Natl Acad Sci USA 106:17858-17863. https://doi.org/10.1073/pnas.0903474106
4. Yu X, Harden K, Gonzalez L C, et al (2009) The surface protein TIGIT suppresses T cell activation by promoting the generation of mature immunoregulatory dendritic cells. Nat Immunol 10:48-57. https://doi.org/10.1038/ni.1674
5. Shibuya A, Campbell D, Hannum C, et al (1996) DNAM-1, a novel adhesion molecule involved in the cytolytic function of T lymphocytes. Immunity 4:573-581. https://doi.org/10.1016/S1074-7613(00)70060-4
6. Shibuya K, Lanier L L, Phillips J H, et al (1999) Physical and functional association of LFA-1 with DNAM-1 adhesion molecule. Immunity 11:615-623. https://doi.org/10.1016/S1074-7613(00)80136-3
7. Chiang E Y, Almeida P E, Nagata D E de A, et al (2020) CD96 functions as a co-inhiitory receptor to enhance CD8+ T cell activation and effector responses. Eur J Immunol 4-6. https://doi.org/10.1111/1744-1633.12020
8. Zhu Y, Paniccia A, Schulick A C, et al (2016) Identification of CD112R as a novel checkpoint for human T cells. J Exp Med 213:167-176. https://doi.org/10.1084/jem.20150785
9. Vaena D, Patnaik A, Hamilton E, et al (2019) Abstract CT168: Phase I study of COM701 (a novel checkpoint inhibitor of PVRIG) in patients with advanced solid tumors. In: Clinical Trials. American Association for Cancer Research, p Abstract CT168
10. Johnston R J, Comps-Agrar L, Hackney J, et al (2014) The Immunoreceptor TIGIT Regulates Antitumor and Antiviral CD8+ T Cell Effector Function. Cancer Cell 26:923-937. https://doi.org/10.1016/j.ccell.2014.10.018
11. Chauvin J, Pagliano O, Fourcade J, et al (2015) TIGIT and PD-1 impair tumor antigen—specific CD8+ T cells in melanoma patients. J Clin Invest 125:2046-2058. https://doi.org/10.1172/JCI80445
12. Harjunpää H, Guillerey C (2020) TIGIT as an emerging immune checkpoint. Clin Exp Immunol 200:108-119. https://doi.org/10.1111/cei.13407
13. Golan T, Bauer T M, Golan T, et al (2018) Phase 1 dose-finding study of the anti—TIGIT antibody MK-7684 as monotherapy and in combination with pembrolizumab in patients with advanced solid. SITC 2018 Abstract 025
14. Sharma S, Ulahannan S, Mettu N B, et al (2018) Initial results from a phase 1a/b study of Etigilimab (OMP-313M32), an anti-T cell immunoreceptor with Ig and ITIM domains (TIGIT) antibody in advanced solid tumours. SITC 2018 Abstract P289
15. Johanna C. Bendell, Bedard P, Bang Y-J, et al (2020) Phase Ia/Ib dose-escalation study of the anti-TIGIT antibody tiragolumab as a single agent and in combination with atezolizumab in patients with advanced solid tumors. 2020 AACR Virtual Annu Meet Abstract CT302
16. Rodriguez-Abreu D, Johnson M L, Hussein M A, et al (2020) Primary analysis of a randomized, double-blind, phase II study of the anti-TIGIT antibody tiragolumab (tira) plus atezolizumab (atezo) versus placebo plus atezo as first-line (1L) treatment in patients with PD-L1-selected NSCLC (CITYSCAPE). J Clin Oncol 38:9503-9503. https://doi.org/10.1200/jco.2020.38.15_supp1.9503
17. Finnefrock A C, Fu T-M, Freed D C, et al (2012) PD-1 Binding Proteins. 2
18. Murter B, Pan X, Ophir E, et al (2019) Mouse PVRIg has CD8+ T cell-specific coinhibitory functions and dampens antitumor immunity. Cancer Immunol Res 7:244-256. https://doi.org/10.1158/2326-6066.CIR-18-0460
19. Savas P, Virassamy B, Ye C, et al (2018) Single-cell profiling of breast cancer T cells reveals a tissue-resident memory subset associated with improved prognosis. Nat Med 24:986-993. https://doi.org/10.1038/s41591-018-0078-7
20. Sun Y, Luo J, Chen Y, et al (2020) Combined evaluation of the expression status of CD155 and TIGIT plays an important role in the prognosis of LUAD (lung adenocarcinoma). Int Immunopharmacol 80:106198. https: //doi.org/10.1016/j.intimp.2020.106198
21. Farhood B, Najafi M, Mortezaee K (2019) CD8+ cytotoxic T lymphocytes in cancer immunotherapy: A review. J Cell Physiol 234:8509-8521. https://doi.org/10.1002/j cp.27782
22. Miller B C, Sen D R, Abosy R Al, et al (2019) Subsets of exhausted CD8+ T cells differentially mediate tumor control and respond to checkpoint blockade. Nat Immunol 20:326-336. https://doi.org/10.1038/s41590-019-0312-6
23. Schietinger A, Philip M, Krisnawan V E, et al (2016) Tumor-Specific T Cell Dysfunction Is a Dynamic Antigen-Driven Differentiation Program Initiated Article Tumor-Specific T Cell Dysfunction Is a Dynamic Antigen-Driven Differentiation Program Initiated Early during Tumorigenesis. Immunity 45:389-401. https://doi.org/10.1016/j.immuni.2016.07.011
24. Philip M, Schietinger A (2019) ScienceDirect Heterogeneity and fate choice: T cell exhaustion in cancer and chronic infections. Curr Opin Immunol 58:98-103. https://doi.org/10.1016/j.coi.2019.04.014
25. Speiser D E, Ho P, Verdeil G (2016) Regulatory circuits of T cell function in Cancer. Nat Rev Immunol 16:599-611. https://doi.org/10.1038/nri.2016.80
26. Yost K E, Satpathy A T, Wells D K, et al (2019) Clonal replacement of tumor-specific T cells following PD-1 blockade. Nat Med 25:1251-1259. https://doi.org/10.1038/s41591-019-0522-3
27. Fourcade J, Sun Z, Chauvin J M, et al (2018) CD226 opposes TIGIT to disrupt Tregs in melanoma. JCI insight 3:1-13. https://doi.org/10.1172/jci.insight.121157
28. Nishiwada S, Sho M, Yasuda S, et al (2015) Clinical significance of CD155 expression in human pancreatic cancer. Anticancer Res 35:2287-97
29. Stamm H, Oliveira-ferrer L, Grossjohann E, et al (2019) Targeting the TIGIT-PVR immune checkpoint axis as novel therapeutic option in breast cancer. Oncoimmunology 8:e1674605. https://doi.org/10.1080/2162402X.2019.1674605
30. Leipold D, Prabhu S (2019) Pharmacokinetic and Pharmacodynamic Considerations in the Design of Therapeutic Antibodies. 130-139. https://doi.org/10.1111/cts.12597
31. Kurtulus S, Sakuishi K, Ngiow S, et al (2015) TIGIT predominantly regulates the immune response via regulatory T cells. J Clin Invest 1:1-10. https://doi.org/10.1172/JCI81187DS1
32. Chihara N, Madi A, Kondo T, et al (2018) Induction and transcriptional regulation of the co-inhibitory gene module in T cells. Nature 558:454-459
33. Nimmerjahn F, Ravetch J V. (2008) Fcγ receptors as regulators of immune responses. Nat Rev Immunol 8:34-47. https://doi.org/10.1038/nri2206

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed:

1. A method of activating NK-cells comprising administering an anti-PVRIG and anti-TIGIT antibody, wherein administering the combination of an anti-PVRIG and anti-TIGIT antibody results in increased activation of NK-cells, optionally as compared to the level of NK-cell activation exhibited for individual administration of an anti-PVRIG or an anti-TIGIT antibody and/or as compared to a control or standard level of NK-cell activation and/or as compared to unactivated NK-cells level.

2. The method of claim 1, wherein the NK-cell activation finds use for the treatment of cancer.

3. The method of claims 1 to 2, wherein the anti-PVRIG antibody binds a human PVRIG, and wherein the anti-TIGIT antibody binds human TIGIT.

4. The method of any one of claims 1 to 3, wherein the NK-cell activation when both an anti-PVRIG and anti-TIGIT antibody are administered is one-fold, two-fold, three-fold, four-fold, five-fold, or more as compared to the level of NK-cell activation exhibited for individual administration of an anti-PVRIG or an anti-TIGIT antibody, and/or as compared to a control or standard level of NK-cell activation, and/or as compared to unactivated NK-cells level.

5. The method of any one of claims 1 to 3, wherein the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG or an anti-TIGIT antibody, and/or as compared to a control or standard level of NK-cell activation, and/or as compared to unactivated NK-cells level.

6. The method of any one of claims 1 to 3, wherein the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody, and/or as compared to a control or standard level of NK-cell activation, and/or as compared to unactivated NK-cells level.

7. The method of any one of claims 1 to 3, wherein the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody, and/or as compared to a control or standard level of NK-cell activation, and/or as compared to unactivated NK-cells level, wherein PVRL2 is expressed on the cancer cells of the individual to which the anti-PVRIG and anti-TIGIT antibodies are being administered.

8. The method of any one of claims 1 to 3, wherein the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-TIGIT antibody, and/or as compared to a control or standard level of NK-cell activation, and/or as compared to unactivated NK-cells level.

9. The method of any one of claims 1 to 3, wherein the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-TIGIT antibody, and/or as compared to a control or standard level of NK-cell activation, and/or as compared to unactivated NK-cells level, wherein PVR is expressed on the cancer cells of the individual to which the anti-PVRIG and anti-TIGIT antibodies are being administered.

10. The method of any one of claims 1 to 3, wherein the NK-cell activation when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell activation exhibited for individual administration of an anti-PVRIG antibody, and/or as compared to a control or standard level of NK-cell activation, and/or as compared to unactivated NK-cells level, wherein PVRL2 is expressed on the cancer cells of the individual to which the anti-PVRIG and anti-TIGIT antibodies are being administered.

11. The method of any one of claims 1 to 10, wherein the NK-cells exhibit increased cytotoxicity when both an anti-PVRIG and anti-TIGIT antibody are administered.

12. The method of claim 11, wherein the NK-cell increased cytotoxicity when both an anti-PVRIG and anti-TIGIT antibody are administered is one-fold, two-fold, three-fold, four-fold, five-fold, or more as compared to the level of NK-cell cytotoxicity exhibited for individual administration of an anti-PVRIG or an anti-TIGIT antibody.

13. The method of claim 11, wherein the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG or an anti-TIGIT antibody.

14. The method of claim 11, wherein the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG antibody.

15. The method of claim 11, wherein the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-PVRIG antibody, wherein PVRL2 is expressed on the cancer cells of the individual to which the anti-PVRIG and anti-TIGIT antibodies are being administered.

16. The method of claim 11, wherein the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-TIGIT antibody.

17. The method of claim 11, wherein the NK-cell increased cytotoxicity when both an anti-PVRIG and anti-TIGIT antibody are administered is increased by 10%, increased by 20%, increased by 30%, increased by 40%, increased by 50%, increased by 60%, increased by 70%, increased by 80%, increased by 90%, increased by 100%, or more as compared to the level of NK-cell cytotoxicity exhibited for individual administration of an anti-TIGIT antibody, wherein PVR is expressed on the cancer cells of the individual to which the anti-PVRIG and anti-TIGIT antibodies are being administered.

18. The method of any one of claims 1 to 17, wherein the NK-cell activation is measured based on an increase in proliferation of at least a subset of NK-cells.

19. The method of any one of claims 1 to 17, wherein the NK-cell activation is measured by increase in expression of activation markers.

20. The method of claim 19, wherein the activation markers include CD69, CD107a, granzyme, and/or perforin.

21. The method of any one of claims 1 to 17, wherein the NK-cell activation is measured based on an increase in immunostimulatory activity.

22. The method of any one of claims 1 to 17, wherein the NK-cell activation is measured based on an increase in cytokine secretion.

23. The method of claim 22, wherein the cytokines include IFNγ and/or TNF.

24. The method of any one of claims 1 to 17, wherein the NK-cell activation is measured based on an increase in direct killing of target cells by NK-cells in vitro.

25. The method of any one of claims 1 to 17, wherein the NK-cell activation is measured based on an increase in direct killing of target cells by NK-cells in vivo.

26. The method of any one of claims 1 to 17, wherein the NK-cell activation is measured based on cell surface receptor expression of CD25.

27. The method any one of claims 1 to 26, wherein the anti-PVRIG antibody comprises:
a. a heavy chain variable domain comprising a vhCDR1, vhCDR2, and vhCDR3 from an anti-PVRIG antibody; and
b. a light chain variable domain comprising a vlCDR1, vlCDR2, and vlCDR3 from an anti-PVRIG antibody;
wherein the anti-PVRIG antibody in a) and b) is selected from the group consisting of CHA.7.518.4, CHA.7.518.1, CHA.7.518, CHA.7.524 CHA.7.530, CHA.7.538_1, CHA.7.538_2, CHA.7.502, CHA.7.503, CHA.7.506, CHA.7.508, CHA.7.510, CHA.7.512, CHA.7.514, CHA.7.516, CHA.7.518, CHA.7.520.1, CHA.7.520.2, CHA.7.522, CHA.7.524, CHA.7.526, CHA.7.527, CHA.7.528, CHA.7.530, CHA.7.534, CHA.7.535, CHA.7.537, CHA.7.538.1, CHA.7.538.2, CHA.7.543, CHA.7.544, CHA.7.545, CHA.7.546, CHA.7.547, CHA.7.548, CHA.7.549,CHA.7.550, CHA7.538.1.2, CPA.7.021, CPA.7.001, CPA.7.003, CPA.7.004, CPA.7.006, CPA.7.008, CPA.7.009, CPA.7.010, CPA.7.011, CPA.7.012, CPA.7.013, CPA.7.014, CPA.7.015, CPA.7.017, CPA.7.018, CPA.7.019,CPA.7.022, CPA.7.023, CPA.7.024, CPA.7.033, CPA.7.034, CPA.7.036, CPA.7.040, CPA.7.046, CPA.7.047, CPA.7.049, CPA.7.050, CHA.7.518, and the antibodies as depicted in FIGS. 24A-24D and 61A-61P.

28. The method of any one of claims 1 to 26, wherein the anti-TIGIT antibody comprises:
a. a heavy chain variable domain comprising a vhCDR1, vhCDR2, and vhCDR3 from an anti-TIGIT antibody; and
b. a light chain variable domain comprising a vlCDR1, vlCDR2, and vlCDR3 from an anti-TIGIT antibody;
wherein the anti-TIGIT antibody in a) and b) is selected from the group consisting of CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8, the antibodies as depicted in FIGS. 23A-23EE and 62A-62FI.

29. The method of any one of claims 1 to 26, wherein the PVRIG antibody comprises the vlCDR1, vlCDR2, vlCDR3, vhCDR1, vhCDR2, and vhCDR3 from CHA.7.518.1.H4(S241P) and the TIGIT antibody comprises the vlCDR1, vlCDR2, vlCDR3, vhCDR1, vhCDR2, and vhCDR3 from CPA.9.086.H4(S241P).

30. The method of any one of claims 1 to 26, wherein the PVRIG antibody is CHA.7.518.1.H4(S241P) and the TIGIT antibody is CPA.9.086.H4(S241P).

31. The method of any one of claims 1 to 30, wherein the anti-PVRIG antibody and/or the anti-TIGIT comprises:
a. a heavy chain comprising VH-CH1-hinge-CH2-CH3; and
b. a light chain comprising VL-CL, wherein the CL is the constant domain of either a kappa or lambda antibody.

32. The method of any one of claims 1 to 30, wherein the CL is kappa.

33. The method of any one of claims 1 to 30, wherein the CL is lambda.

34. The method of any one of claims 1 to 33, wherein the anti-PVRIG antibody and/or the anti-TIGIT antibody is a humanized antibody.

35. The method of any one of claims 2 to 34, wherein the cancer is selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, neuroendocrine lung cancer (including neuroendocrine lung carcinoma), NSCLC, NSCL (large cell), NSCLC large cell, NSCLC squamous cell, cervical SCC, malignant melanoma, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, and Myelodysplastic syndromes (MDS).

36. The method of claim 35, wherein the cancer is selected from the group consisting of triple negative breast cancer, stomach (gastric) cancer, Acute myeloid leukemia (AML), lung cancer (small cell lung, non-small cell lung), Merkel Cells cancer, MSI-high cancer, KRAS mutant tumors, adult T-cell leukemia/lymphoma, myeloma, and Myelodysplastic syndromes (MDS).

37. The method of any one of claims 2 to 34, wherein the cancer is selected from the group consisting of advanced cancer, solid tumor, neoplasm malignant, ovarian cancer, breast cancer, lung cancer, endometrial cancer, ovarian neoplasm, triple negative breast cancer, lung neoplasm, colorectal cancer, endometrial neoplasms, and ovarian cancer.

38. The method of any one of claims 1 to 37, wherein the cancer is AML.

39. The method of any one of claims 1 to 37, wherein the individual has AML cancer cells that are PVRL2^{hi}PVR^{low} and/or PVRL2⁺PVR^{low}.

40. The method of claim 38, wherein the AML cancer cells are PVRL2^{hi}PVR^{low} and/or PVRL2⁺PVR^{low}.

41. The method of claim 39 or 40, wherein the AML cancer cells are AML blasts.

42. The method of any one of claims 1 to 41, wherein the AML is selected from the group consisting of AML with minimal differentiation (M0), AML without maturation (M1), AML with maturation (M2), Acute Promeyelocitic Leukemia (M3), Acute myelomonocytic leukemia (M4), Acute monoblastic/monocytic leukemia (M5a/b), Acute Erythroleukemia (M6), Acute Megakaryocytic Leukemia (M7), Acute basophilic leukemia, Acute panmyelosis with myelofibrosis, therapy related AML (Alkylating agent related AML or Topoisomerase II inhibitor related), AML with myelodysplasia related changes (AMLMRC), AML with myelodysplasia related changes, myeloid sarcoma, myeloid proliferations related to Down syndrome (transient abnormal myelopoeisis or myeloid leukemia associated with Down syndrome), blastic plasmacytoid dentritic cell neoplasm, acute leukemia of ambiguous lineage, and AML with recurrent genetic abnormalities.

43. The method of claim 42, wherein the acute leukemia of ambiguous lineage is selected from the group consisting of acute undifferentiated leukemia, mixed phenotype acute leukemia with t(9;22)(q34;q11.2) (BCR-ABL1), mixed phenotype acute leukemia with t(v;11q23) (MLL rearranged), mixed phenotype acute leukemia (B/myeloid, NOS), mixed phenotype acute leukemia (T/myeloid, NOS), mixed phenotype acute leukemia (NOS, rare types), and other acute leukemia of ambiguous lineage.

44. The method of claim 43, wherein the AML with recurrent genetic abnormalities is selected from the group consisting of AML with t(8;21)(q22;q22) (RUNX1-RUNX1T1), AML with inv(16)(p13.1;q22) or t(16;16)(p13.1;q22) (CBF&beta-MYH11), Acute promyelocytic leukemia with t(15;17)(q22;q12) (PML/RAR&alpha and variants), AML with t(9;11)(p22;q23) (MLLT3-MLL), AML with t(6;9)(p23;q34) (DEK-NUP214), AML with inv(3)(q21q26.2) or t(3;3)(q21;q26.2) (RPN1-EVI1), AML (megakaryoblastic) with t(1;22)(p13;q13) (RBM15-MKL1), AML with mutated NPM1, and AML with mutated CEBPA.

45. The method of any one of claims 1 to 44, wherein the AML is related to specific mutations in one or more genes that are selected from the group consisting of FLT3, NPM1, IDH1/2, DNMT3A, KMT2A, RUNX1, ASXL, and TP53.
